(19) United States
(12) Patent Application Publication
(10) Pub. No.: US 2010/0240638 A1
(43) Pub. Date: Sep. 23, 2010

(54) Organic Compounds and their uses

(76) Inventors: Shawn D. Britt, Andover, MA (US); Jiping Fu, Arlington, VA (US); David Thomas Parker, Lexington, MA (US); Michiael Patane, Andover, MA (US); Parkash Raman, Acton, MA (US); Branko Radetich, Boston, MA (US); Mohindra Seepersaud, Belmont, MA (US); Aregahegn Yifru, Somerville, MA (US); Rui Zheng, Arlington, VA (US); Trixi Brand, Basel (CH); Sylvain Cottens, Andover (CH); Claus Ehrhardt, Loerrach (DE); Stefan Andreas Randl, Basel (CH); Pascal Rigollier, Mulhouse (FR); Nikolaus Schiering, Weilam Rhein (DE); Oliver Simic, Basel (DE)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 12/527,787

(22) PCT Filed: Feb. 19, 2008
(86) PCT No.: PCT/EP08/01281

§ 371 Date: Mar. 8, 2010

Related U.S. Application Data
(60) Provisional application No. 60/890,754, filed on Feb. 20, 2007.

(51) Int. Cl. C07D 285/00; A61K 38/13; A61K 31/33; A61K 31/00; A61K 31/551; A61K 31/5025; A61K 31/5377; A61K 31/50; A61K 31/496; A61K 31/497; A61K 31/519; A61K 31/4375; A61K 31/437; A61K 31/47; A61K 31/4523; A61K 31/4427; A61K 31/403; A61P 31/12; A61P 35/04; A61P 1/16; A61P 37/00

(52) U.S. Cl. 514/218; 514/183; 514/228.5; 514/233.2; 514/248; 514/249; 514/252.13; 514/264.1; 514/264.11; 514/300; 514/312; 514/322; 514/338; 514/414; 540/455

(57) ABSTRACT

The present application describes macrocyclic compounds of formula (I) with NS3 protease inhibitory activity for treating hepatitis C virus infection.

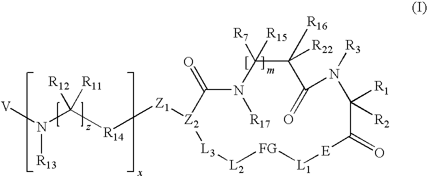

### BACKGROUND

Chronic hepatitis C virus (HCV) infection is a major global health burden, with an estimated 170 million people infected worldwide and an additional 3 to 4 million infected each year (See e.g. World Health Organization Fact Sheet No.164. October 2000). Although 25% of new infections are symptomatic, 60-80% of patients will develop chronic liver disease, of whom an estimated 20% will progress to cirrhosis with a 1-4% annual risk of developing hepatocellular carcinoma (See e.g. World Health Organization Guide on Hepatitis C. 2002; Pawlotsky, J-M. (2006) Therapy of Hepatitis C: From Empiricism to Eradication. Hepatology 43:S207-S220). Overall, HCV is responsible for 50-76% of all liver cancer cases and two thirds of all liver transplants in the developed world (See e.g. World Health Organization Guide on Viral Cancers. 2006). And ultimately, 5-7% of infected patients will die from the consequences of HCV infection (See e.g. World Health Organization Guide on Hepatitis C. 2002).

The current standard therapy for HCV infection is pegylated interferon alpha (IFN-α) in combination with ribavirin. However, only up to 50% of patients with genotype 1 virus can be successfully treated with this interferon-based therapy. Moreover, both interferon and ribavirin can induce significant adverse effects, ranging from flu-like symptoms (fever and fatigue), hematologic complications (leukopenia, thrombocytopenia), neuropsychiatric issues (depression, insomnia, irritability), weight loss, and autoimmune dysfunctions (hypothyroidism, diabetes) from treatment with interferon to significant hemolytic anemia from treatment with ribavirin. Therefore, more effective and better tolerated drugs are still greatly needed.

HCV, first identified in 1989 (See e.g. Choo, Q. L. et al. Science (1989) 244:359-362), is a single-stranded RNA virus with a 9.6-kilobase genome of positive polarity. It encodes a single polyprotein that is cleaved upon translation by cellular and viral proteases into at least ten individual proteins: C, E1, E2, p7, NS2, NS3, NS4A, NS4B, NS5A, and NS5B (See e.g. Lindenbach, B. D. et al. (2001). Flaviviridae: the viruses and their replication, p. 991-1041. In D. M. Knipe, P. M. Howley, and D. E. Griffin (ed.), Fields virology, 4th ed, vol. 1. Lippincott Williams & Wilkins, Philadelphia, Pa.).

NS3, an approximately 70 kDa protein, has two distinct domains: a N-terminal serine protease domain of 180 amino acids (AA) and a C-terminal helicase/NTPase domain (AA 181 to 631). The NS3 protease is considered a member of the chymotrypsin family because of similarities in protein sequence, overall three-dimensional structure and mechanism of catalysis. The HCV NS3 serine protease is responsible for proteolytic cleavage of the polyprotein at the NS3/NS4A, NS4A/NS4B, NS4B/NS5A and NS5A/NS5B junctions (See e.g. Bartenschlager, R., L. et al. (1993) J. Virol. 67:3835-3844; Grakoui, A. et al. (1993) J. Virol. 67:2832-2843; Tomei, L. et al. (1993) J. Virol. 67:4017-4026). NS4A, an approximately 6 kDa protein of 54 AA, is a co-factor for the serine protease activity of NS3 (See e.g. Failla, C. et al. (1994) J. Virol. 68:3753-3760; Tanji, Y. et al. (1995) J. Virol. 69:1575-1581). Autocleavage of the NS3/NS4A junction by the NS3/NS4A serine protease occurs intramolecularly (i.e., cis) while the other cleavage sites are processed intermolecularly (i.e., trans). It has been demonstrated that HCV NS3 protease is essential for viral replication and thus represents an attractive target for antiviral chemotherapy.

### SUMMARY OF THE INVENTION

There remains a need for new treatments and therapies for HCV infection, as well as HCV-associated disorders. There is also a need for compounds useful in the treatment or prevention or amelioration of one or more symptoms of HCV, as well as a need for methods of treatment or prevention or amelioration of one or more symptoms of HCV. Furthermore, there is a need for methods for modulating the activity of HCV-serine proteases, particularly the HCV NS3/NS4a serine protease, using the compounds provided herein.

In one aspect, the invention provides compounds of the Formula I:

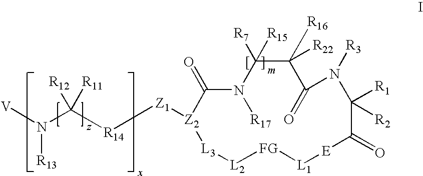

and pharmaceutically acceptable salts, enantiomers, stereoisomers, rotamers, tautomers, diastereomers, or racemates thereof;

wherein
the macrocycle:

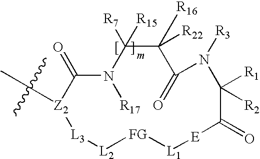

comprises between 15 to 40 ring atoms;

m, x and z are each independently selected from 0 or 1;

p is selected at each occurrence from the group consisting of 0, 1 and 2;

R₁ and R₂ are independently selected, at each occurrence, from hydrogen or cyano, or from the group consisting of alkyl, alkenyl, alkynyl, cycloalkyl, cycloalkylalkyl, alkoxy, and cycloalkyloxy, each of which is unsubstituted or substituted with 1-6 moieties which can be the same or different and are independently selected from the group consisting of hydroxy, oxo, alkyl, aryl, alkoxy, aryloxy, thio, alkylthio, arylthio, amino, alkylamino, arylamino, alkylsulfonyl, arylsulfonyl, alkylsulfonamido, arylsulfonamido, heteroarylsulfonamido, arylaminosulfonyl, heteroarylaminosulfonyl, mono and dialkylaminosulfonyl, carboxy, carbalkoxy, amido, carboxamido, alkoxycarbonylamino, aminocarbonyloxy, alkoxycarbonyloxy, alkylureido, arylureido, halogen, cyano, or nitro; wherein each of said alkyl, alkoxy, and aryl can be unsubstituted or optionally independently substituted with one or more moieties which can be the same or different and are independently selected from alkyl, alkenyl, alkynyl, cycloalkyl, cycloalkyl-alkyl, heterocyclyl, heterocyclylalkyl, aryl, alkylaryl, aralkyl, arylheteroaryl, heteroaryl, heterocyclylamino, alkylheteroaryl and heteroaralkyl;

R₃ is selected from the group consisting of H and C₁₋₄-alkyl;

E is a divalent residue selected from the group consisting of C(O)NR₂₃, NR₂₃S(O)ₚ, NR₂₃S(O)ₚNR₂₃;

L₁ and L₂ are divalent residues independently selected from the group consisting of C₀₋₄alkylene, (CH₂)ᵢ—FG-(CH₂)ₖ, (CH₂)ᵢ—C₃₋₇cycloalkylene-(CH₂)ₖ, (CH₂)ᵢ—C₃₋₇cycloheteroalkylene-(CH₂)ₖ, alkenylene, alkynylene, arylene, heteroarylene, cycloalkylene and heterocycloalkylene, each of which is substituted with 0 to 4 independently selected X₁ or X₂ groups;

i and k are independently selected integers of from 0 to 7;

L₃ is a C₀₋₄alkylene or a divalent ethylene or acetylene residue, wherein the C₀₋₄alkylene and divalent ethylene residues are substituted by 0-2 substituents selected from alkyl, aryl, heteroaryl, mono- or di-alkylamino-C₀-C₆alkyl, hydroxyl alkyl or alkoxyalkyl;

FG is absent or a divalent residue selected from the group consisting of O, S(O)ₚ, NR₂₃, C(O), C(O)NR₂₃, NR₂₃C(O), OC(O)NR₂₃, NR₂₃C(O)O, NR₂₃C(O)NR₂₃, S(O)ₚNR₂₃, NR₂₃S(O)ₚ, and NR₂₃S(O)ₚNR₂₃;

R₂₃ is independently selected at each occurrence from hydrogen or the group consisting of alkyl, alkenyl, alkynyl, cycloalkyl, cycloalkylalkyl, aryl, heteroaryl, heteroaralkyl, aralkyl and heteroaralkyl, each of which is substituted with 0-2 substituents independently selected from halogen, alkyl, alkoxy, and mono- and di-alkylamino; or

Two R₂₃ residues, taken in combination, form a monocyclic, bicyclic or tricyclic heterocyclic ring system which is saturated, partially unsaturated, or aromatic, and which is substituted with 0 to 3 substituents independently selected from C₁₋₆alkyl, C₁₋₆alkoxy, C₁₋₆alkoxyC₁₋₆alkoxy, mono- and di-C₁₋₆alkylaminoC₁₋₆alkoxy, C₁₋₆haloalkyl, C₁₋₆haloalkoxy, mono- and di-C₁₋₆alkylamino, halogen, 4 to 7 member heterocycloalkyl, aryl, heteroaryl, and 3 to 6 member spirocycloalkyl or spiroheterocycloalkyl, each of which is substituted with 0 to 3 substituents independently selected from the group consisting of C₁₋₄alkyl, C₁₋₄alkoxy, hydroxy, amino, and mono- and di-C₁₋₄alkylamino;

R₉ is absent or selected from hydrogen, C₁₋₄alkyl, C₃₋₇cycloalkyl-C₀₋₄alkyl, or hydroxy;

R₇, R₁₀, R₁₁, R₁₂, R₁₃, R₁₅, R₁₆, R₁₇, and R₂₂ are each, independently, hydrogen or selected from the group consisting of alkyl, alkenyl, alkynyl, aryl, alkyl-aryl, heteroalkyl, heterocyclyl, heteroaryl, aryl-heteroaryl, alkyl-heteroaryl, cycloalkyl, alkyloxy, alkyl-aryloxy, aryloxy, heteroaryloxy, heterocyclyloxy, cycloalkyloxy, amino, alkylamino, arylamino, alkyl-arylamino, arylamino, heteroarylamino, cycloalkylamino, carboxyalkylamino, aralkyloxy and heterocyclylamino; each of which may be further substituted 0 to 5 times with substituents independently selected from X₁ and X₂;

X₁ is alkyl, alkenyl, alkynyl, cycloalkyl, cycloalkyl-alkyl, heterocyclyl, heterocyclylalkyl, aryl, alkylaryl, aralkyl, arylheteroaryl, heteroaryl, heterocyclylamino, alkylheteroaryl, or heteroaralkyl; wherein X₁ can be independently substituted with one or more of X₂ moieties which can be the same or different and are independently selected;

X₂ is hydroxy, oxo, alkyl, aryl, heteroaryl, alkoxy, aryloxy, heteroaryloxy, thio, alkylthio, arylthio, heteroarylthio, amino, alkylamino, arylamino, heteroarylamino, alkylsulfonyl, arylsulfonyl, heteroarylsulfonyl, alkylsulfonamido, arylsulfonamido, heteroarylsulfonamido, arylaminosulfonyl, heteroarylaminosulfonyl, mono and dialkylaminosulfonyl, carboxy, carbalkoxy, amido, carboxamido, alkoxycarbonylamino, aminocarbonyloxy, alkoxycarbonyloxy, carbamoyl, ureido, alkylureido, arylureido, halogen, cyano, or nitro; wherein each of said alkyl, alkoxy, and aryl can be unsubstituted or optionally independently substituted with one or more moieties which can be the same or different and are independently selected from alkyl, alkenyl, alkynyl, cycloalkyl, cycloalkyl-alkyl, heterocyclyl, heterocyclylalkyl, aryl, alkylaryl, aralkyl, aryiheteroaryl, heteroaryl, heterocyclylamino, alkylheteroaryl and heteroaralkyl;

Z₁ is C₀₋₄alkylene, oxygen or NR₁₀;

Z₂ is CR₉, O or N;

R₁₄ is C(O) or S(O)ₚ;

V is selected from hydrogen or from the group consisting of alkyl, alkyl-aryl, heteroalkyl, heterocyclyl, heteroaryl, aryl-heteroaryl, alkyl-heteroaryl, cycloalkyl, alkyloxy, alkyl-aryloxy, aryloxy, heteroaryloxy, heterocyclyloxy, cycloalkyloxy, amino, alkylamino, arylamino, alkyl-arylamino, arylamino, heteroarylamino, cycloalkylamino, carboxyalkylamino, mono- and di-alkylcarboxamide, aralkyloxy and heterocyclylamino; each of which may be further independently substituted one or more times with X¹ and X²; wherein X¹ is alkyl, alkenyl, alkynyl, cycloalkyl, cycloalkyl-alkyl, heterocyclyl, heterocyclylalkyl, aryl, alkylaryl, aralkyl, aryloxy, arylthio, aryiheteroaryl, heteroaryl, heterocyclylamino, alkylheteroaryl, or heteroaralkyl; wherein X¹ can be independently substituted with one or more X² moieties which can be the same or different and are independently selected; wherein X² is hydroxy, oxo, alkyl, cycloalkyl, spirocycloalkyl, heterocycloalkyl, aryl, heteroaryl, alkoxy, aryloxy, thio, alkylthio, amino, mono- and di-alkylamino, arylamino, alkylsulfonyl, arylsulfonyl, alkylsulfonamido, arylsulfonamido, carboxy, carbalkoxy, carboxamido, alkoxycarbonylamino, alkoxycarbonyl, alkoxycarbonyloxy, alkylureido, arylureido, halogen, cyano, or nitro; wherein each X₂ residue selected to be alkyl, alkoxy, and aryl can be unsubstituted or optionally independently substituted with one or more moieties which can be the same or different and are independently selected from alkyl, alkenyl, alkynyl, cycloalkyl, cycloalkyl-alkyl, heterocyclyl, heterocyclylalkyl, aryl, alkylaryl, aralkyl, aryiheteroaryl, heteroaryl, heterocyclylamino, alkylheteroaryl and heteroaralkyl;

or V is selected from the group consisting of -Q¹-Q², wherein Q¹ is absent, C(O), S(O)₂, N(H), N(C₁₋₄-alkyl), C═N(CN), C═N(SO₂CH₃), C═N—COH—C₁₋₄-alkyl, or C═N—COH, and Q² is hydrogen or is selected from the group consisting of C₁₋₄-alkyl, O—C₁₋₄-alkyl, NH₂, N(H)—C₁₋₄-alkyl, N(C₁₋₄-alkyl)₂, SO₂-aryl, SO₂-heteroaryl, SO₂—C₁₋₄-alkyl, C₃₋₆cycloalkyl-C₀₋₄-alkyl, aryl, heteroaryl and heterocycle, each of which may be independently substituted one or more times with a halogen atom, C₁₋₄-alkyl, C₁₋₄-alkyl substituted by one or more halogen atoms, or C₃₋₆-cycloalkyl;

or R₂₂ and R₁₆ may together form a 3, 4, 5, 6 or 7-membered ring and may contain one or more heteroatoms, wherein the ring may be further substituted one or more times;

or R₇ and R₁₅ may together form a 3, 4, 5, 6 or 7-membered ring and may contain one or more heteroatoms, wherein the ring may be further substituted one or more times;

or R₁₅ and R₁₇ may together form a 3, 4, 5, 6 or 7-membered ring and may contain one or more heteroatoms, wherein the ring may be further substituted one or more times;

or R₁₅ and R₁₆ may together form a 4, 5, 6 or 7-membered ring and may contain one or more heteroatoms, wherein the ring may be further substituted one or more times;

or R₁₅ and R₁₆ may together form an arylene or heteroarylene ring and R₇ and R₂₂ are absent, wherein the ring may be further substituted one or more times;

or R₁ and R₂ may together form a 3, 4, 5, 6 or 7-membered ring that is saturated or partially unsaturated and may contain one or more heteroatoms, which ring is substituted with 0-3 residues independently selected from C₁₋₄alkyl, C₁₋₄alkoxy, C₂₋₄alkenyl, C₂₋₄alkynyl, halogen, hydroxy, C₃₋₆cylcoalkyl and C₃₋₆spirocycloalkyl;

or R₁₇ and R₁₆ may together form a 4, 5, 6, 7 or 8-membered ring of the formula:

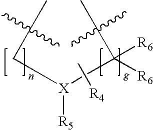

wherein

n and g are each, independently, 0, 1 or 2;

X is O, S, N, C or CR₅ₐ;

R₄ is hydrogen or is selected from the group consisting of C₁₋₆-alkyl, C₃₋₇-cycloalkyl, aryl, heterocycle and heteroaryl, all of which may be independently substituted one or more times with a halogen atom or C₁₋₄-alkyl;

R₅ is absent, hydrogen or oxo or is selected from the group consisting of hydroxyl, C₁₋₈-alkyl, C₂₋₈-alkenyl, C₂₋₈-alkynyl, aryl-C₀₋₄-alkyl, heterocycle-C₀₋₄-alkyl, heteroaryl-C₀₋₄-alkyl, C₃₋₈-cycloalkyloxy, aryloxy, NR₂₃COR₂₃, CONR₂₃R₂₃, NR₂₃CONHR₂₃, OCONR₂₃R₂₃, NR₂₃COOR₂₃, OCOR₂₃, COOR₂₃, aryl-C(O)O, aryl-C(O)NR₂₃, heteroaryloxy, heteroaryl-C(O)O, heterocycle-C(O)O, heteroaryl-C(O)NR₂₃, heterocycle-C(O)NR₂₃, each of which may be independently substituted one or more times (or more preferably 0, 1, 2, 3, 4, or 5 times) with halogen, C₁₋₄-alkyl, C₁₋₄-alkoxy, haloC₁₋₄-alkyl, haloC₁₋₄-alkoxy, amino, mono- and di-C₁₋₄alkylaminoC₀₋₄alkyl, mono- and di-C₁₋₄alkylaminoC₀₋₄alkoxy, C₃₋₇cycloalkyl, fused- or spiro-cyclic 3-7 membered ring, heterocycleC₀₋₄alkoxy, heterocycleC₀₋₄alkyl, aryl, or heteroaryl;

R₅ₐ is selected from the group consisting of H, hydroxyl, C₁₋₈-alkyl, C₂₋₈-alkenyl, C₂₋₈-alkynyl, C₃₋₈-cycloalkyl-C₀₋₄-alkyl, aryl-C₀₋₄-alkyl and heteroaryl-C₀₋₄-alkyl,

or R₄ and R₅ may together form a fused dimethyl cyclopropyl ring, a fused cyclopentane ring, a fused phenyl ring or a fused pyridyl ring, each of which may be substituted with a halogen atom, aryl, heteroaryl, trihalomethyl, C₁₋₄-alkoxy or C₁₋₄-alkyl;

or R₅ and R₅ₐ may together form a spirocyclic ring having between 3 and 7 ring atoms and having 0, 1, or 2 ring heteroatoms, which is optionally substituted by 0-4 substitutents selected from cyano, halogen, hydroxyl, amino, thiol, C₁₋₈-alkyl, C₂₋₈-alkenyl, C₂₋₈-alkynyl, C₁₋₈-alkoxy-C₀₋₄alkyl, C₁₋₈-haloalkyl, C₂₋₈-haloalkenyl, C₂₋₈-haloalkynyl, C₁₋₈-haloalkoxy, C₁₋₈-alkylthio, C₁₋₈-alkylsulfonyl, C₁₋₈-alkylsulfoxy, C₁₋₈-alkanoyl, C₁₋₈-alkoxycarbonyl, C₃₋₇-cycloalkyl-C₀₋₄-alkyl, aryl-C₀₋₄-alkyl, heteroaryl-C₀₋₄-alkyl, COOH, C(O)NH₂, mono- and di-C₁₋₄-alkyl-carboxamide, mono- and di-C₁₋₄-alkyl-amino-C₀₋₄alkyl, SO₃H, SO₂NH₂, and mono-and di-C₁₋₄-alkylsulfonamide, or two, substitutents taken together form a fused or spirocyclic 3 to 7 membered ring having 0, 1 or 2 ring heteroatoms selected from N, O and S, which fused or spirocyclic ring has 0 to 2 independently selected substitutents selected from cyano, halogen, hydroxyl, amino, thiol, C₁₋₈-alkyl, C₂₋₈-alkenyl, C₂₋₈-alkynyl, C₁₋₈-alkoxy-C₀₋₄alkyl, C₁₋₈-haloalkyl, C₂₋₈-haloalkenyl, C₂₋₈-haloalkynyl, C₁₋₈-haloalkoxy, C₁₋₈-alkylthio, C₁₋₈-alkylsulfonyl, C₁₋₈-alkylsulfoxy, C₁₋₈-alkanoyl, C₁₋₈-alkoxycarbonyl, C₃₋₇-cycloalkyl-C₀₋₄-alkyl, aryl-C₀₋₄-alkyl, heteroaryl-C₀₋₄-alkyl, COOH, C(O)NH₂, mono- and di-C₁₋₄-alkyl-carboxamide, mono- and di-C₁₋₄-alkyl-amino-C₀₋₄alkyl, SO₃H, SO₂NH₂, and mono-and di-C₁₋₄-alkylsulfonamide; and

R₆ is independently selected at each occurrence from the group consisting of hydrogen, hydroxy, amino, C₁₋₄alkyl, C₁₋₄alkoxy, and mono- and di-C₁₋₄alkylamino, and C₃₋₆cycloalkylC₀₋₄alkyl;

or two R₆ residues may together form a spirocyclic ring having between 3 and 7 ring atoms and having 0, 1, or 2 ring heteroatoms, which is optionally substituted by 0-4 substitutents selected from cyano, halogen, hydroxyl, amino, thiol, C₁₋₈-alkyl, C₂₋₈-alkenyl, C₂₋₈-alkynyl, C₁₋₈-alkoxy-C₀₋₄alkyl, C₁₋₈-haloalkyl, C₂₋₈-haloalkenyl, C₂₋₈-haloalkynyl, C₁₋₈-haloalkoxy, C₁₋₈-alkylthio, C₁₋₈-alkylsulfonyl, C₁₋₈-alkylsulfoxy, C₁₋₈-alkanoyl, C₁₋₈-alkoxycarbonyl, C₃₋₇-cycloalkyl-C₀₋₄-alkyl, aryl-C₀₋₄-alkyl, heteroaryl-C₀₋₄-alkyl, COOH, C(O)NH₂, mono- and di-C₁₋₄-alkyl-carboxamide, mono- and di-C₁₋₄-alkyl-amino-C₀₋₄alkyl, SO₃H, SO₂NH₂, and mono-and di-C₁₋₄-alkylsulfonamide, or two substitutents taken together form a fused or spirocyclic 3 to 7 membered ring having 0, 1 or 2 ring heteroatoms selected from N, O and S, which fused or spirocyclic ring has 0 to 2 independently selected substitutents selected from halogen, C₁₋₄alkyl, C₁₋₄alkoxy, C₁₋₄alkanoyl, mono- and di-C₁₋₄-alkylamino, mono- and di-C₁₋₄-alkyl-carboxamide, C₁₋₄-alkoxycarbonyl, and phenyl.

In one embodiment, the invention provides a method of treating an HCV-associated disorder comprising administering to a subject in need thereof a pharmaceutically acceptable amount of a compound of the invention, such that the HCV-associated disorder is treated.

In another embodiment, the invention provides a method of treating an HIV infection comprising administering to a subject in need thereof a pharmaceutically acceptable amount of a compound of the invention.

In still another embodiment, the invention provides a method of treating, inhibiting or preventing the activity of HCV in a subject in need thereof, comprising administering to the subject a pharmaceutically acceptable amount of a compound of the invention. In one embodiment, the compounds of the invention inhibit the activity of the NS2 protease, the NS3 protease, the NS3 helicase, the NS5a protein, and/or the NS5b polymerase. In another embodiment, the interaction between the NS3 protease and NS4A cofactor is disrupted. In yet another embodiment, the compounds of the invention prevent or alter the severing of one or more of the NS4A-NS4B, NS4B-NS5A and NS5A-NS5B junctions of the HCV. In another embodiment, the invention provides a method of inhibiting the activity of a serine protease, comprising the step of contacting said serine protease with a compound of the invention. In another embodiment, the invention provides a method of treating, inhibiting or preventing the activity of HCV in a subject in need thereof, comprising administering to the subject a pharmaceutically acceptable amount of a compound of the invention, wherein the compound interacts with any target in the HCV life cycle. In one embodiment, the target of the HCV life cycle is selected from the group consisting of NS2 protease, NS3 protease, NS3 helicase, NS5a protein and NS5b polymerase.

In another embodiment, the invention provides a method of decreasing the HCV RNA load in a subject in need thereof comprising administering to the subject a pharmaceutically acceptable amount of a compound of the invention.

In another embodiment, the compounds of the invention exhibit HCV protease activity. In one embodiment, the compounds are an HCV NS3-4A protease inhibitor.

In another embodiment, the invention provides a method of treating an HCV-associated disorder in a subject, comprising administering to a subject in need thereof a pharmaceutically acceptable amount of a compound of the invention, and a pharmaceutically acceptable carrier, such that the HCV-associated disorder is treated.

In another embodiment, the invention provides a method of treating an HCV-associated disorder in a subject wherein the subject is suffering from or susceptible to a viral infection which is resistant to one or more anti-viral therapies, the method comprising administering to a subject in need thereof a pharmaceutically acceptable amount of a compound of the invention, and a pharmaceutically acceptable carrier, such that the drug-resistant HCV-associated disorder is treated.

In still another embodiment, the invention provides a method of treating an HCV-associated disorder comprising administering to a subject in need thereof a pharmaceutically effective amount of a compound of the invention, in combination with a pharmaceutically effective amount of an additional HCV-modulating compound, such as interferon or derivatized interferon, or a cytochrome P450 monooxygenase inhibitor, such that the HCV-associated disorder is treated. In one embodiment, the additional HCV-modulating compound is selected from the group consisting of ITMN191, Sch 503034 and VX-950.

In another embodiment, the invention provides a method of inhibiting hepatitis C virus replication in a cell, comprising contacting said cell with a compound of the invention.

In yet another embodiment, the invention provides a packaged HCV-associated disorder treatment, comprising an HCV-modulating compound of the invention, packaged with instructions for using an effective amount of the HCV-modulating compound to treat an HCV-associated disorder.

In certain embodiments, the HCV-associated disorder is selected from the group consisting of HCV infection, liver cirrhosis, chronic liver disease, hepatocellular carcinoma, cryoglobulinaemia, non-Hodgkin's lymphoma, and a suppressed innate intracellular immune response.

In another embodiment, the invention provides a method of treating HCV infection, liver cirrhosis, chronic liver disease, hepatocellular carcinoma, cryoglobulinaemia, non-Hodgkin's lymphoma, and/or a suppressed innate intracellular immune response in subject in need thereof comprising administering to the subject a pharmaceutically acceptable amount of a compound of the invention.

In one embodiment, the HCV to be treated is selected of any HCV genotype. In another embodiment, the HCV is selected from HCV genotype 1, 2 and/or 3.

### DETAILED DESCRIPTION OF THE INVENTION

This invention is directed to compounds, e.g., peptide compounds, and intermediates thereto, as well as pharmaceutical compositions containing the compounds for use in treatment of HCV infection. This invention is also directed to the compounds of the invention or compositions thereof as protease inhibitors, particularly as serine protease inhibitors, and more particularly as HCV NS3 protease inhibitors. The compounds are particularly useful in interfering with the life cycle of the hepatitis C virus and in treating or preventing an HCV infection or physiological conditions associated therewith. The present invention is also directed to methods of combination therapy for inhibiting HCV replication in cells, or for treating or preventing an HCV infection in patients using the compounds of the invention or pharmaceutical compositions, or kits thereof.

In one aspect, the compounds of the invention are compounds of Formula I, in which R₁ and R₂ taken in combination form a 3, 4, 5, or 6-membered saturated carbocyclic ring which is substituted with 0-2 substituents independently selected from halogen, alkyl, alkenyl, alkoxy and C₃₋₆cycloalkyl. In other aspects, compounds of the invention are compounds of Formula I, in which R₁ and R₂ taken in combination form a cyclopropyl ring. In certain compounds of Formula I include those compounds in which R₁ and R₂ are taken in combination to form a cyclopropyl ring substituted with 0-2 substituents independently selected from halogen, alkyl, alkenyl, and alkoxy or substituted with 0 to 2 C₁-C₄alkyl residues. Still other compounds of Formula I include those in which R₁ and R₂ are taken in combination to form a cyclopropyl ring which is substituted with 0 or 1 substituents selected C₁₋₄alkyl, vinyl or cyclopropyl; and E is C(O)NH, NHS(O)₂, NHSO₂N(Me), NHSO₂N(Et) or NHSO₂N(cyclopropyl).

In another aspect, the compounds of the invention are compounds of any one of Formulae I, in which R₁ is H or C₁ alkyl; and R₂ is H, C₁-C₄alkyl, C₁-C₄fluoroalkyl, C₂-C₄alkenyl, or C₃-C₇cycloalkyl C₀₋₂alkyl.

Certain other compounds of Formula I comprise a macrocycle having between 15 and 40 ring atoms, between 15 and 35, 15 and 30 or 15 and 25 ring atoms, or between 17 and 23 ring atoms. Certain compounds of Formula I comprise a macrocycle having 15, 16, 17, 18, 19, 20, 21, 22, 23, 24, or 25 ring atoms. In certain instances, compounds of Formula I comprise a macrocycle having 16, 17, 18, 19, 20, 21, 22, or 23 ring atoms.

Certain other compounds of Formula I comprise a macrocycle selected from the group consisting of macrocycles of the formulae:

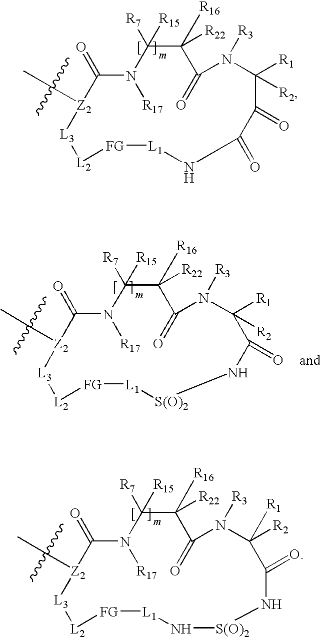

In certain compounds of Formula I, L₁ is C₁-C₆alkylene, C₃-C₇cycloalkylene, arylene or heteroarylene, each of which is substituted by 0-4 residues independently selected from C₁-C₄alkyl, C₁-C₄alkoxy, hydroxyl, amino, mono- and di-C₁-C₄alkylamino, halogen, cyano, C₁-C₄fluoroalkyl, C₁-C₄fluoroalkoxy, COOH, carboxamide (CONH₂), mono- and di-C₁-C₄alkylcarboxamide, aryl, heteroaryl and 5 or 6 membered saturated heterocycles;

L₂ is selected from C₁-C₆alkylene and C₂-C₆alkenylene, each of which is substituted by 0-4 residues independently selected from C₁-C₄alkyl, C₁-C₄alkoxy, hydroxyl, amino, mono- and di-C₁-C₄alkylamino, halogen, cyano, C₁-C₄fluoroalkyl, C₁-C₄fluoroalkoxy, COOH, carboxamide (CONH₂), mono- and di-C₁-C₄alkylcarboxamide, aryl, heteroaryl and 5 or 6 membered saturated heterocycles; and

L₃ is absent or a divalent ethylene residue which is substituted by 0 to 2 independently selected methyl or ethyl residues.

In yet other compounds of Formula I, L₁ is a divalent residue selected from C₂-C₄alkylene, 1,2-phenylene, 1,3-phenylene, 2,4-pyridylene, 2,3-pyridylene, 3,4-pyridylene or 1,7-indolylene, 2,7-indolylene, each of which is substituted with 0-3 residues selected from C₁-C₄alkyl, C₁-C₄alkoxy, hydroxyl, amino, mono- and di-C₁-C₄alkylamino, halogen, cyano, C₁-C₂fluoroalkyl, C₁-C₂fluoroalkoxy, COOH, carboxamide (CONH₂), and mono- and di-C₁-C₄alkylcarboxamide.

In certain compounds of Formula I, L₁ is C₃-C₇cycloalkylene, arylene or heteroarylene which is substituted by 0-4 residues independently selected from C₁-C₄alkyl, C₁-C₄alkoxy, hydroxyl, amino, mono- and di-C₁-C₄alkylamino, halogen, cyano, C₁-C₄fluoroalkyl, C₁-C₄fluoroalkoxy, COOH, carboxamide (CONH₂), mono- and di-C₁-C₄alkylcarboxamide, aryl, heteroaryl and 5 or 6 membered saturated heterocycles;

L₂ is selected from C₁-C₆alkylene and C₂-C₆alkenylene, each of which is substituted by 0-4 residues independently selected from C₁-C₄alkyl, C₁-C₄alkoxy, hydroxyl, amino, mono- and di-C₁-C₄alkylamino, halogen, cyano, C₁-C₄fluoroalkyl, C₁-C₄fluoroalkoxy, COOH, carboxamide (CONH₂), mono- and di-C₁-C₄alkylcarboxamide, aryl, heteroaryl and 5 or 6 membered saturated heterocycles; and

L₃ is absent or a divalent ethylene residue which is substituted by 0 to 2 independently selected methyl or ethyl residues.

In yet other compounds of Formula I, L₁ is a divalent residue selected from 1,2-phenylene, 1,3-phenylene, 2,4-pyridylene, 2,3-pyridylene, 3,4-pyridylene or 1,7-indolylene, 2,7-indolylene, each of which is substituted with 0-3 residues selected from C₁-C₄alkyl, C₁-C₄alkoxy, hydroxyl, amino, mono- and di-C₁-C₄alkylamino, halogen, cyano, C₁-C₂fluoroalkyl, C₁-C₂fluoroalkoxy, COOH, carboxamide (CONH₂), and mono- and di-C₁-C₄alkylcarboxamide.

Certain compounds of Formula I include compounds of Formula II:

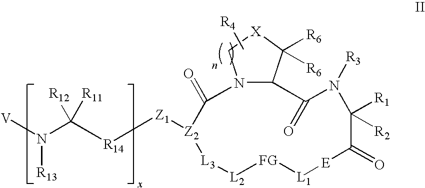

and pharmaceutically acceptable salts, enantiomers, stereoisomers, rotamers, tautomers, diastereomers, or racemates thereof.

Yet other compounds of the invention according to Formula II include those compounds in which:

x is 0 or 1;

n is 0 or 1;

R₁₄ is C(O) or S(O)ₚ;

Z₁ is absent or NH;

Z₂ is nitrogen or CH;

R₁ is selected from the group consisting of H and C₁₋₄-alkyl;

R₂ is selected from the group consisting of C₁₋₄-alkyl, C(O)C₁₋₄-alkyl, C(O)OC₁₋₄-alkyl, and (CH₂)₀₋₄—C₃₋₆-cycloalkyl;

or R₁ and R₂ together form a cyclopropane ring;

R₃ is selected from the group consisting of H and C₁₋₄-alkyl;

X is O, NR₅ or CR₅R₅ₐ;

R₄ is hydrogen or is selected from the group consisting of C₁₋₄-alkyl, C₃₋₆-cycloalkyl, aryl, heterocycle and heteroaryl, each of which may be independently substituted one or more times with a halogen atom or C₁₋₄-alkyl;

R₅ is hydrogen or oxo or is selected from the group consisting of hydroxyl, C₁₋₈-alkyl, C₂₋₈-alkenyl, C₂₋₈-alkynyl, C₃₋₈-cycloalkyl-C₀₋₄-alkyl, aryl-C₀₋₄-alkyl, aryloxy, heteroaryloxy, heterocycle-C₀₋₄-alkyl and heteroaryl-C₀₋₄-alkyl, each of which may be independently substituted one or more times with a halogen atom, aryl, heteroaryl, trihalomethyl, C₁₋₄-alkoxy or C₁₋₄-alkyl;

R₅ₐ is selected from the group consisting of H, hydroxyl, C₁₋₈-alkyl, C₂₋₈-alkenyl, C₂₋₈-alkynyl, C₃₋₈-cycloalkyl-C₀₋₄-alkyl, aryl-C₀₋₄-alkyl and heteroaryl-C₀₋₄-alkyl,

or R₄ and R₅ may together form a fused dimethyl cyclopropyl ring, a fused cyclopentane ring, a fused phenyl ring or a fused pyridyl ring, each of which may be substituted with a halogen atom, aryl, heteroaryl, trihalomethyl, C₁₋₄-alkoxy or C₁₋₄-alkyl;

or R₅ and R₅ₐ may together form a spirocarbocyclic saturated ring having between 3 and 6 carbon ring atoms which is optionally substituted by 0-2 substitutents selected from halogen, C₁₋₆-alkyl, C₂₋₆-alkenyl, C₂₋₆-alkynyl, C₁₋₆-alkoxide, C₃₋₇-cycloalkyl-C₀₋₄-alkyl, phenyl-C₀₋₄-alkyl, naphthyl-C₀₋₄-alkyl, heteroaryl-C₀₋₄-alkyl, or two substitutents taken together form a fused or spirocyclic 3 to 7 membered carbocyclic ring, each of which is substituted with 0-3 independently selected halogen atoms or C₁₋₄-alkyl groups;

R₁₀ and R₁₁ are each, independently, selected from the group consisting of H and C₁₋₄-alkyl;

R₆ and R₁₃ is H;

R₁₂ is selected from the group consisting of H, C₁₋₄-alkyl and C₃₋₆-cycloalkyl; and

V is selected from the group consisting of -Q¹-Q², wherein Q¹ is absent, C(O), N(H), N(C₁₋₄-alkyl), C═N(CN), C═N(SO₂CH₃), or C═N—COH, and Q² is H, C₁₋₄-alkyl, C═N—COH—C₁₋₄-alkyl, C₁₋₄-alkoxy, C₃₋₇cycloalkyloxy, heterocycloalkyloxy, NH₂, N(H)—C₁₋₄-alkyl, N(C₁₋₄-alkyl)₂, SO₂-aryl, SO₂—C₁₋₄alkyl, C₃₋₆-cycloalkyl-C₀₋₄alkul, aryl, heteroaryl and heterocycle, each of which may be independently substituted one or more times with a halogen atom, C₁₋₄-alkyl, C₁₋₄alkoxy, C₂-C₄alkenyloxy, C₂-C₄alkynyloxy, C₁₋₄-alkyl substituted by one or more halogen atoms, or C₃₋₆-cycloalkyl;

or when x is 0, R₁₀ and V can form a cyclopropyl ring that may be further substituted by an amide group.

Still other compounds of the invention according to Formula II include those compounds in which X is CR₅R₅ₐ, R₄ is H, and R₅ and 12₅ₐ taken in combination form a 3 to 6 member spirocyclic carbocycle substituted with 0-2 substitutents selected from halogen, C₁₋₆-alkyl, C₂₋₆-alkenyl, C₂₋₆-alkynyl, C₁₋₆-alkoxide, C₃₋₇-cycloalkyl-C₀₋₄-alkyl, phenyl-C₀₋₄-alkyl, naphthyl-C₀₋₄-alkyl, heteroaryl-C₀₋₄-alkyl, or two substitutents taken together form a fused or spirocyclic 3 to 7 membered carbocyclic ring, each of which is substituted with 0-3 independently selected halogen atoms or C₁₋₄-alkyl groups.

Yet other compounds of the invention according to Formula II include compounds according to Formula IIa:

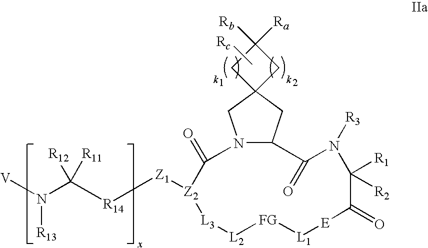

wherein
Z₂ is nitrogen or CH;
k₁ and k₂ are 0 or 1 such that a sum of k₁ and k₂ equals 1 or 2;
Rₐ is hydrogen, C₁₋₄alkyl, or phenyl;
R_{b} is hydrogen, C₁₋₄alkyl, C₁₋₄alkoxy-C₀₋₄alkyl, mono- and di-C₁₋₄alkylaminoC₀₋₄alkyl, mono- and di-C₁₋₄alkyl carboxamide, C₁₋₄alkanoyl, C₁₋₄alkoxycarbonyl, or phenyl
or Rₐ and R_{b} taken together form a fused or spirocyclic 3 to 6 membered ring having 0, 1 or 2 ring heteroatoms selected from N, O and S, which fused or spirocyclic ring has 0 to 2 independently selected substitutents selected from halogen, C₁₋₄alkyl, C₁₋₄alkoxy, C₁₋₄alkanoyl, and phenyl; and
R_{c} represents 0 to 4 substitents which are independently selected at each occurrence of R_{c} from the group consisting of halogen, C₁₋₄alkyl, and phenyl, or two geminal R_{c} substitents, taken in combination form a 3 to 6 member spirocyclic ring.

Certain compounds of the invention according to Formula IIa include those compounds in which the divalent residue:

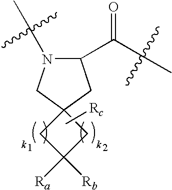

is selected from the group consisting of:

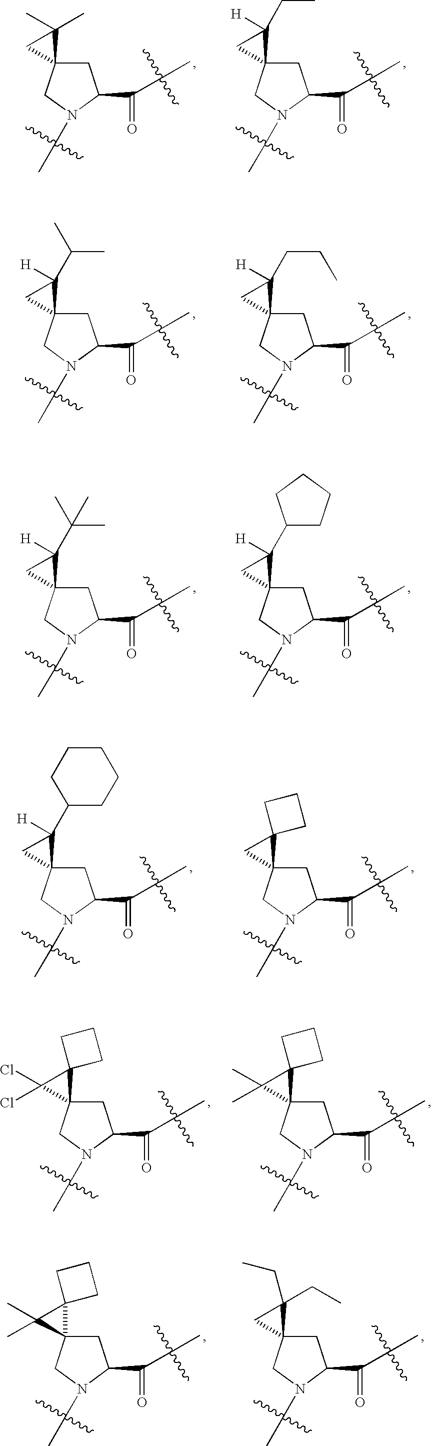

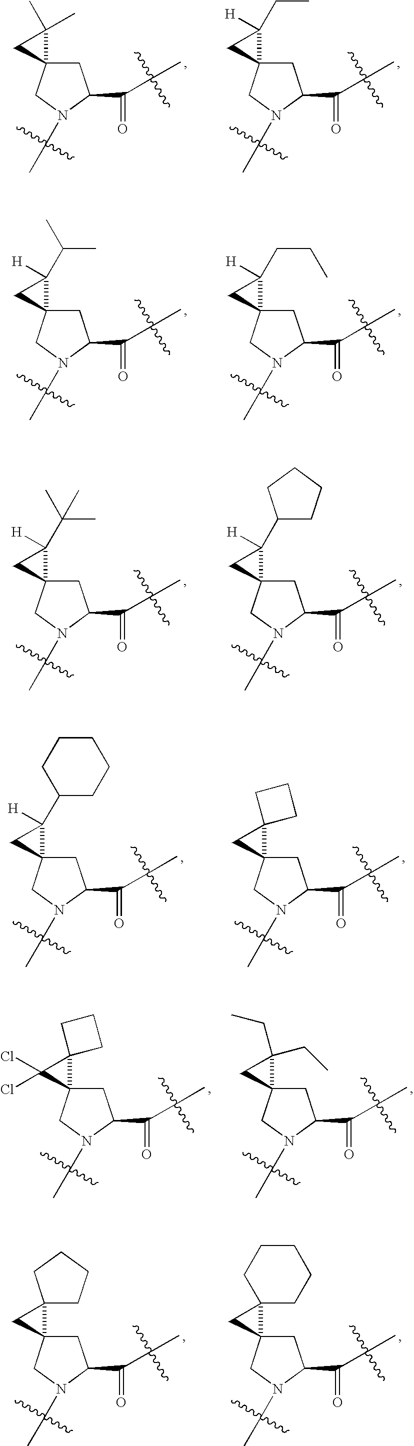

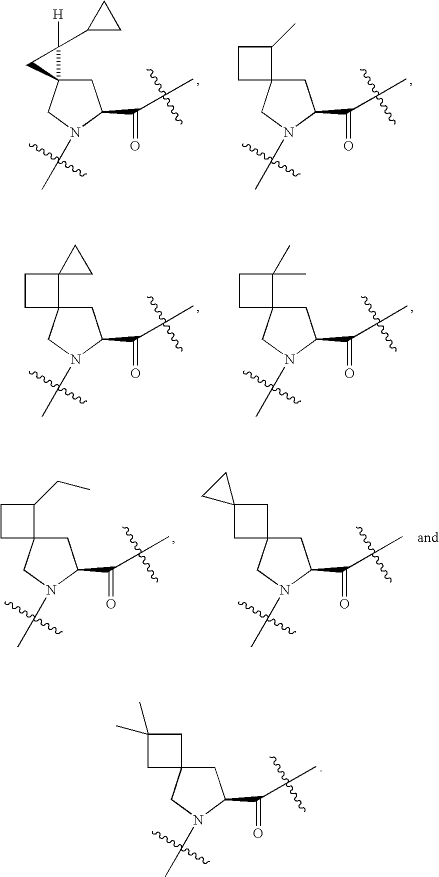

Yet other compounds of the invention according to Formula II include those compounds in which: X is CR₅R₅ₐ; and

R₅ and R₅ₐ, taken in combination, form a spirocyclic ring having between 3 and 7 ring atoms and having 0, 1, or 2 ring heteroatoms, which spirocyclic ring is substituted with a spirocyclic 3 to 7 membered ring having 0, 1 or 2 ring heteroatoms selected from N, O and S, and wherein each of the spirocyclic rings has 0 to 2 independently selected substitutents selected from cyano, halogen, hydroxyl, amino, thiol, C₁₋₈-alkyl, C₂₋₈-alkenyl, C₂₋₈-alkynyl, C₁₋₈-alkoxy-C₀₋₄alkyl, C₁₋₈-haloalkyl, C₂₋₈-haloalkenyl, C₂₋₈-haloalkynyl, C₁₋₈-haloalkoxy, C₁₋₈-alkylthio, C₁₋₈-alkylsulfonyl, C₁₋₈-alkylsulfoxy, C₁₋₈-alkanoyl, C₁₋₈-alkoxycarbonyl, C₃₋₇-cycloalkyl-C₀₋₄-alkyl, heteroaryl-C₀₋₄-alkyl, COOH, C(O)NH₂, mono- and di-C₁₋₄-alkyl-carboxamide, mono- and di-C₁₋₄-alkyl-amino-C₀₋₄alkyl, SO₃H, SO₂NH₂, and mono-and di-C₁₋₄-alkylsulfonamide.

Certain other compounds according to Formula I or Formula II include those compounds in which X is CR₅R₅ₐ wherein R₅ₐ is hydrogen, methyl or trifluoromethyl; and R₅ is a residue of the formula:

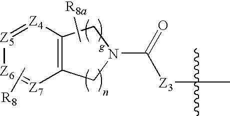

wherein

n and g are integers independently selected from 0, 1, or 2 (preferably n+g=1, 2, 3 or 4; or more preferably n+g is 2 or 3);

Z₃ is NR₂₃ or O;

Z₄, Z₅, Z₆, and Z₇ are each independently selected from the group consisting of N, CH, and CR₈; and

R₈ and R₈ₐ each indepently represent 0 to 2 groups, each of which is independently selected at each occurrence of R₈ and R₈ₐ from the group consisting of hydrogen, halogen, C₁₋₄-alkyl, C₁₋₄-alkoxy, haloC₁₋₄-alkyl, haloC₁₋₄-alkoxy, amino, mono- and di-C₁₋₄alkylaminoC₀₋₄alkyl, mono- and di-C₁₋₄alkylaminoC₀₋₄alkoxy, heterocycleC₀₋₄alkoxy, heterocycleC₀₋₄alkylamino and heterocycleC₀₋₄alkyl; or

two R₈ₐ, taken in combination, form a fused- or spiro-cyclic 3-7 membered ring.

Yet other compounds of Formula I or Formula II include those compounds in which X is CR₅ₐ, R₅ₐ is hydrogen or methyl, and R₅ is a residue selected from the group consisting of:

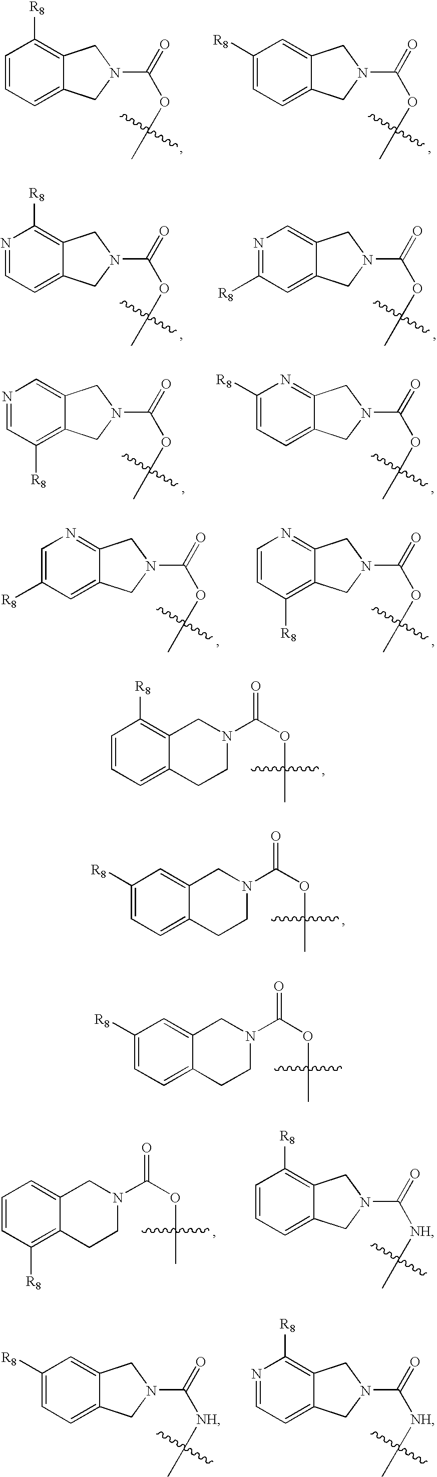

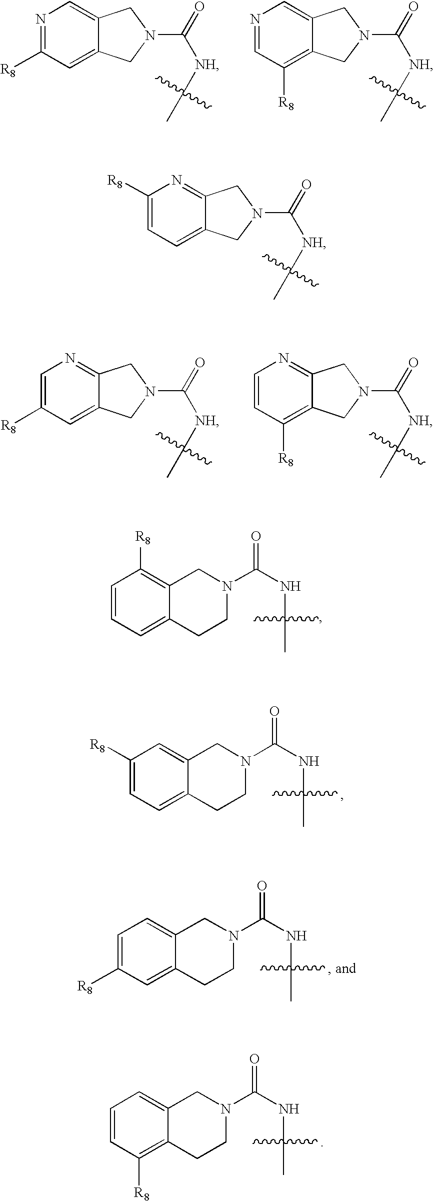

wherein R₈ is selected from hydrogen, methyl, ethyl, mono-, di-, or tri-fluoromethyl, mono-, di-, or tri-fluoromethoxy, fluoro, and chloro.

In still other compounds of Formula I or Formula II include those compounds in which the residue

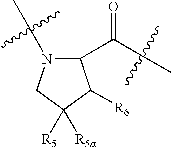

is a residue of the formula:

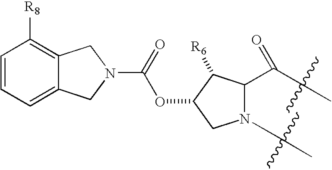

wherein wherein R₆ is hydrogen, methyl, ethyl, and mono-, di-, and tri-fluoromethyl; R₈ is selected from R₈ is selected from hydrogen, methyl, ethyl, mono-, di-, or tri-fluoromethyl, mono-, di-, or tri-fluoromethoxy, fluoro, and chloro.

Still other compounds of Formula I or Formula II include those compounds in which X is CR₅ₐ, R₅ₐ is hydrogen or methyl, and R₅ is a residue selected from the group consisting of:

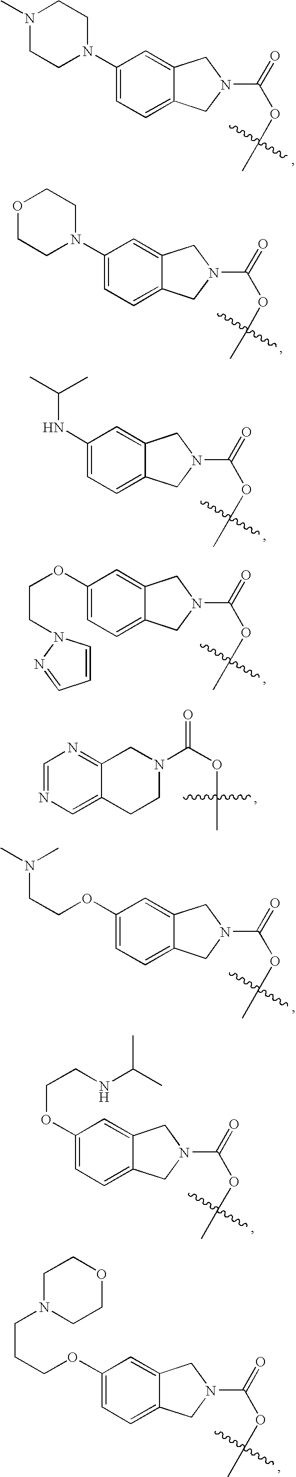

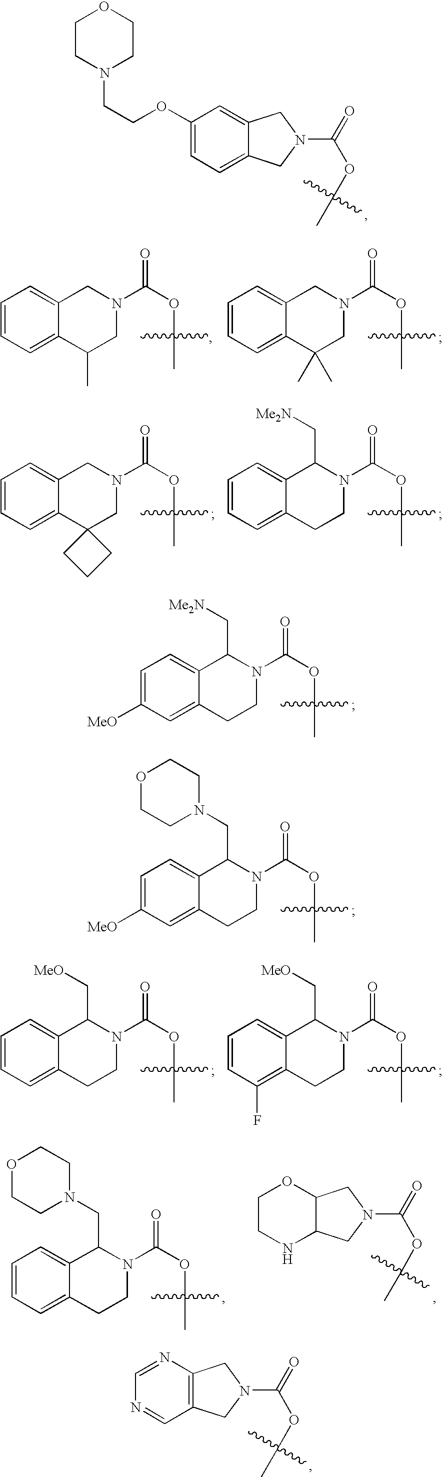

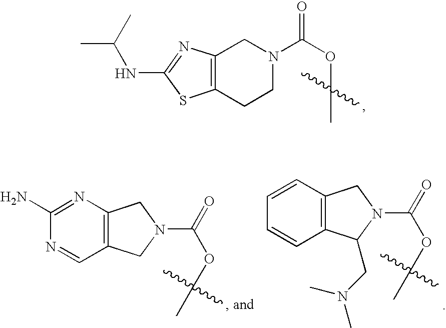

Still other compounds of the invention according to Formula II include compounds according to Formula IIb:

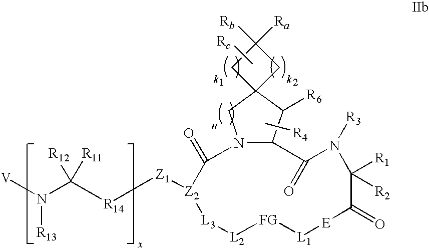

Z₂ is nitrogen or CH;
k₁ and k₂ are 0 or 1 such that a sum of k₁ and k₂ equals 1 or 2;
Rₐ and R_{b} taken together form a spirocyclic 3 to 6 membered ring having 0, 1 or 2 ring heteroatoms selected from N, O and S, which fused or spirocyclic ring has 0 to 2 independently selected substituents selected from halogen, C₁₋₄alkyl, C₁₋₄alkoxy, C₁₋₄alkanoyl, and phenyl;
R_{c} represents 0 to 2 substituents which are independently selected at each occurrence of R_{c} from the group consisting of halogen, C₁₋₄alkyl, and phenyl, or two geminal R_{c} substitents, taken in combination form a 3 to 6 member spirocyclic ring;
R₄ represents 0, 1, or 2 substituents each of which is independently selected from H and C₁₋₄-alkyl; and
R₆ is hydrogen or C₁₋₄alkyl.

In certain compounds of the invention according to Formula IIb, the divalent residue:

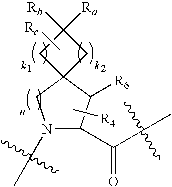

is selected from the group consisting of:

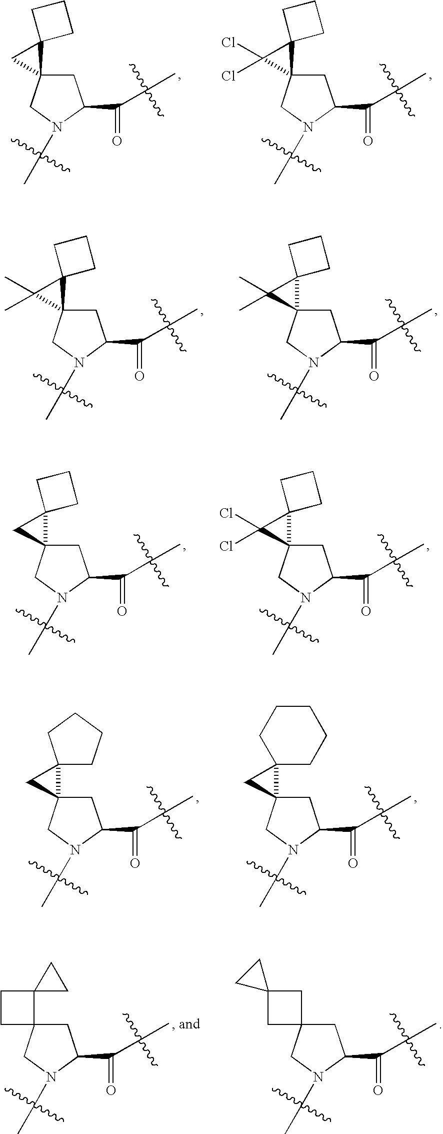

Certain compounds of Formula II, include those compounds in which the

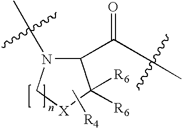

ring is a divalent residue derived from a proline residue selected from the group consisting of:

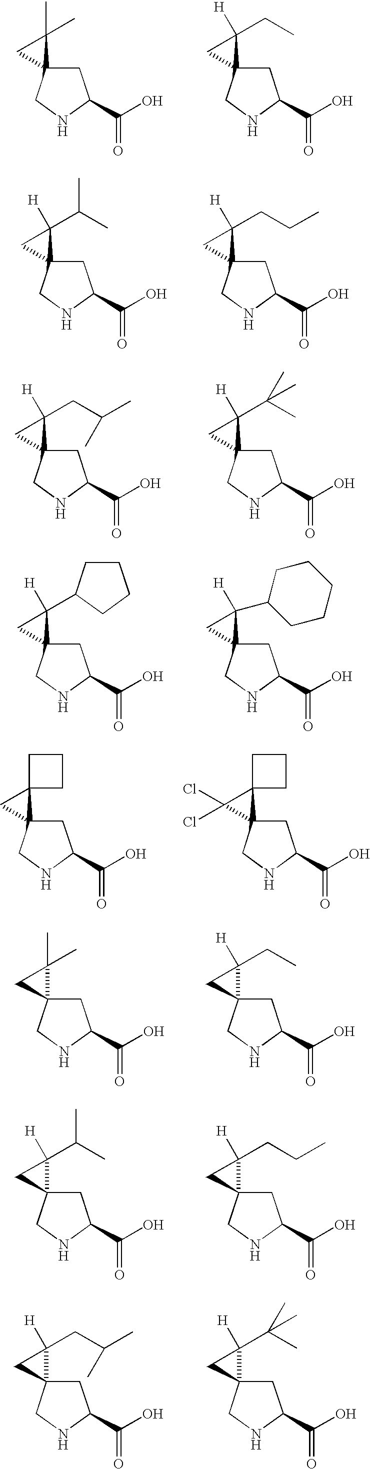

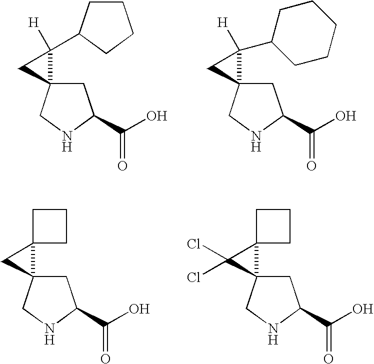

Certain other compounds of Formula II, Formula IIa or Formula IIb include compounds in which X is CR₅R₅ₐ, R₄ is H, and R₅ and R₅ₐ taken in combination form a 3 to 6 member spirocyclic carbocycle substituted with 0-2 substitutents selected from halogen, C₁₋₆-alkyl, C₂₋₆-alkenyl, C₂₋₆-alkynyl, C₁₋₆-alkoxide, C₃₋₇-cycloalkyl-C₀₋₄-alkyl, phenyl-C₀₋₄-alkyl, naphthyl-C₀₋₄-alkyl, heteroaryl-C₀₋₄-alkyl, or two substitutents taken together form a fused or spirocyclic 3 to 7 membered carbocyclic ring, each of which is substituted with 0-3 independently selected halogen atoms or C₁₋₄-alkyl groups.

Certain compounds of Formulae I include compounds of Formula III:

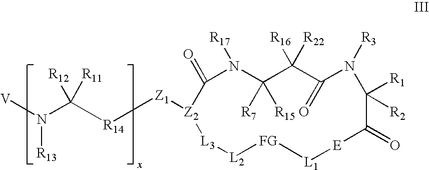

and pharmaceutically acceptable salts, enantiomers, stereoisomers, rotamers, tautomers, diastereomers, or racemates thereofs.

Certain compounds of the invention according to Formula III include compounds in which:

Z₁ is absent or NR₁₀;

Z₂ is nitrogen or CH;

R₃ is selected from the group consisting of H, C₁₋₄-alkyl, and C₃₋₆-cycloalkyl C₀-C₄alkyl;

R₁₁, R₁₅ and R₂₂ are selected from the group consisting of H, alkyl-aryl, C₁₋₄-alkyl, O—C₁₋₄-alkyl, N(H)—C₁₋₄-alkyl, and C₃₋₆-cycloalkyl C₀-C₄alkyl;

R₁₀ and R₁₇ are each, independently, selected from the group consisting of H, C₁₋₄-alkyl and (CH₂)₀₋₄—C₃₋₆-cycloalkyl; or

R₁₅ and R₁₆ may together form a 3, 4, 5, 6 or 7-membered ring that may comprise between 0 to 3 additional heteroatoms, wherein the ring may be further substituted with 0-5 substitutents; or

R₁₆ and R₁₇ may together form a 3, 4, 5, 6 or 7-membered ring that may comprise between 0 to 3 additional heteroatoms, wherein the ring may be further substituted with 0-5 substitutents; and

V is selected from the group consisting of -Q¹-Q², wherein Q¹ is absent, C(O), N(H), N(C₁₋₄-alkyl), C═N(CN), C═N(SO₂CH₃), or C═N—COH, and Q² is H, C₁₋₄-alkyl, C═N—COH—C₁₋₄-alkyl, O—C₁₋₄-alkyl, NH₂, N(H)—C₁₋₄-alkyl, N(C₁₋₄-alkyl)₂, SO₂-aryl, SO₂—C₁₋₄-alkyl, C₃₋₆-cycloalkyl-C₀₋₄-alkyl, aryl, heteroaryl and heterocycle, each of which may be independently substituted one or more times with a halogen atom, C₁₋₄-alkyl, C₁₋₄alkoxy, C₂-C₄alkenyloxy, C₂-C₄alkynyloxy, C₁₋₄-alkyl substituted by one or more halogen atoms, or C₃₋₆-cycloalkyl;

Certain other compounds of the invention according to Formula III include compounds in which:

R₃ is selected from the group consisting of H and C₁₋₄-alkyl;

R₁₃ is H;

R₈, R₁₀ and R₁₁ are each, independently, selected from the group consisting of H, C₁₋₄-alkyl, and C₃₋₇cycloalkyl C₀₋₄alkyl;

R₁₂ is selected from the group consisting of H, C₁₋₄-alkyl and (CH₂)₀₋₄—C₃₋₆-cycloalkyl; and

V is selected from the group consisting of -Q¹-Q², wherein Q¹ is absent, C(O), N(H), N(C₁₋₄-alkyl), C═N(CN), C═N(SO₂CH₃), or C═N—COH, and Q² is H, C₁₋₄-alkyl, C═N—COH—C₁₋₄-alkyl, O—C₁₋₄-alkyl, NH₂, N(H)—C₁₋₄-alkyl, N(C₁₋₄-alkyl)₂, SO₂-aryl, SO₂—C₁₋₄-alkyl, C₃₋₆-cycloalkyl-C₀₋₄-alkyl, aryl, heteroaryl and heterocycle, each of which may be independently substituted one or more times with a halogen atom, C₁₋₄-alkyl, C₁₋₄-alkyl substituted by one or more halogen atoms, C₁₋₄alkoxy, C₂-C₄alkenyloxy, C₂-C₄alkynyloxy, or C₃₋₆-cycloalkyl.

Certain compounds of Formula III include compounds represented by Formula IIIa:

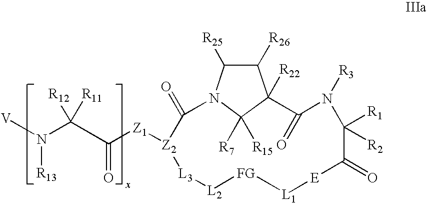

and pharmaceutically acceptable salts, enantiomers, stereoisomers, rotamers, tautomers, diastereomers, or racemates thereof;
wherein

Z₂ is nitrogen or CH;

R₂₅ and R₂₆ are each, independently, selected from the group consisting of H, C₁₋₄-alkyl, O—C₁₋₄-alkyl, N(R₂₄)₂, C₃₋₆cycloalkylC₀-C₄alkyl, substituted or unsubstituted aryl and substituted or unsubstituted heterocycle, wherein each R₂₄ is independently selected from the group consisting of H, halogen, hydroxy, COOH, amino, carboxamide, substituted or unsubstituted-C₁₋₄-alkyl, substituted or unsubstituted C₃₋₆cycloalkylC₀-C₄alkyl, substituted or unsubstituted-C₁₋₄-alkoxy, substituted or unsubstituted C₃₋₆cycloalkylC₀-C₄alkyl-oxy-, substituted or unsubstituted arylC₀-C₄alkyl, substituted or unsubstituted heterocycleC₀-C₄alkyl, substituted or unsubstituted arylC₀-C₄alkyl-oxy and substituted or unsubstituted heterocycleC₀-C₄alkyl-oxy;

or R₂₂ or R₂₆ may together form a 3-membered ring that is substituted or unsubstituted.

In another embodiment of Formula IIIa, R₂₅ is H and R₂₆ is amine, substituted or unsubstituted phenyl, or substituted or unsubstituted benzyl.

Certain other compounds of Formula III include compounds represented by Formula IIIb:

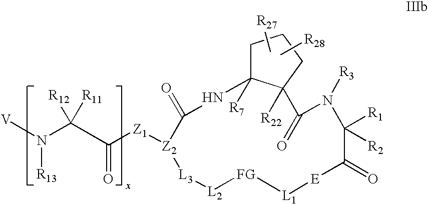

and pharmaceutically acceptable salts, enantiomers, stereoisomers, rotamers, tautomers, diastereomers, or racemates thereof;
wherein

Z₂ is nitrogen or CH;

R₂₇ and R₂₈ are each, independently, selected from the group consisting of H, C₁₋₄-alkyl, O—C₁₋₄-alkyl, N(R₂₄)₂, C₃₋₆cycloalkylC₀-C₄alkyl, substituted or unsubstituted aryl, substituted or unsubstituted O-aryl and substituted or unsubstituted heterocycle, wherein R₂₄ is independently selected at each occurrence from the group consisting of H, halogen, hydroxy, COOH, amino, carboxamide, substituted or unsubstituted-C₁₋₄-alkyl, substituted or unsubstituted C₃₋₆cycloalkylC₀-C₄alkyl, substituted or unsubstituted-C₁₋₄-alkoxy, substituted or unsubstituted C₃₋₆cycloalkylC₀-C₄alkyl-oxy-, substituted or unsubstituted arylC₀-C₄alkyl, substituted or unsubstituted heterocycleC₀-C₄alkyl, substituted or unsubstituted arylC₀-C₄alkyl-oxy and substituted or unsubstituted heterocycleC₀-C₄alkyl-oxy.

In one embodiment of Formula IIIb, R₂₈ is quinoline, C₁₋₄-alkyl, O—C₁₋₄-alkyl, or O-quinoline, wherein the quinoline and O-quinoline substituents may be independently substituted one or more times (or preferably between one and five times) with halogen, amino, O—C₁₋₄-alkyl, substituted or unsubstituted-C₁₋₄-alkyl, substituted or unsubstituted-(CH₂)₀₋₄—C₃₋₆-cycloalkyl, substituted or unsubstituted aryl, substituted or unsubstituted O-aryl, and substituted or unsubstituted heterocycle.

Yet other compounds of Formula III include compounds represented by Formula IIIc:

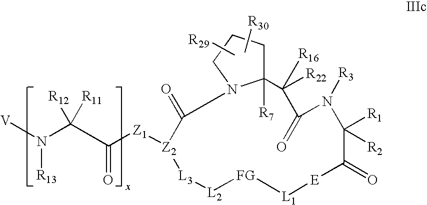

and pharmaceutically acceptable salts, enantiomers, stereoisomers, rotamers, tautomers, diastereomers, or racemates thereof;
wherein

Z₂ is nitrogen or CH;

R₂₉ and R₃₀ are selected from the group consisting of H, C₁₋₄-alkyl, O—C₁₋₄-alkyl, N(R₂₄)₂, C₃₋₆cycloalkylC₀-C₄alkyl, substituted or unsubstituted aryl, substituted or unsubstituted aryl-oxy and substituted or unsubstituted heterocycle, wherein each R₂₄ is independently selected at each occurrence from the group consisting of H, halogen, hydroxy, COOH, amino, carboxamide, substituted or unsubstituted-C₁₋₄-alkyl, substituted or unsubstituted C₃₋₆cycloalkylC₀-C₄alkyl, substituted or unsubstituted-C₁₋₄-alkoxy, substituted or unsubstituted C₃₋₆cycloalkylC₀-C₄alkyl-oxy-, substituted or unsubstituted arylC₀-C₄alkyl, substituted or unsubstituted heterocycleC₀-C₄alkyl, substituted or unsubstituted arylC₀-C₄alkyl-oxy and substituted or unsubstituted heterocycleC₀-C₄alkyl-oxy.

In one embodiment of Formula IIIc, R₂₉ is selected from the group consisting of O-phenyl and O-benzyl.

Still other compounds of Formula III include compounds represented by Formula IIId:

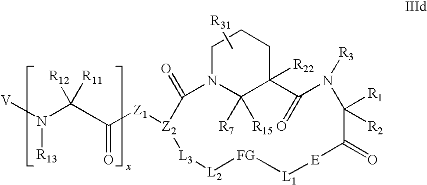

and pharmaceutically acceptable salts, enantiomers, stereoisomers, rotamers, tautomers, diastereomers, or racemates thereof;
wherein

Z₂ is nitrogen or CH;

R₃₁ represents one or two residues which are independently selected at each occurrence from the group consisting of H, C₁₋₄-alkyl, O—C₁₋₄-alkyl, N(R₂₄)₂, (CH₂)₀₋₄—C₃₋₆-cycloalkyl, substituted or unsubstituted aryl, substituted or unsubstituted O-aryl and substituted or unsubstituted heterocycle, wherein each R₂₄ is independently selected from the group consisting of H, halogen, hydroxy, COOH, amino, carboxamide, substituted or unsubstituted-C₁₋₄-alkyl, substituted or unsubstituted C₃₋₆cycloalkylC₀-C₄alkyl, substituted or unsubstituted-C₁₋₄-alkoxy, substituted or unsubstituted C₃₋₆cycloalkylC₀-C₄alkyl-oxy-, substituted or unsubstituted arylC₀-C₄alkyl, substituted or unsubstituted heterocycleC₀-C₄alkyl, substituted or unsubstituted arylC₀-C₄alkyl-oxy and substituted or unsubstituted heterocycleC₀-C₄alkyl-oxy;

or two R₃₁ residues may together form a 3, 4, 5, 6 or 7-membered ring that is aromatic or non-aromatic and may contain one or more heteroatoms selected from N, O or S, wherein the ring may be further substituted one or more times (or preferably between one and five times).

In another embodiment, Formula IIId is represented by a compound of the Formula IIIe:

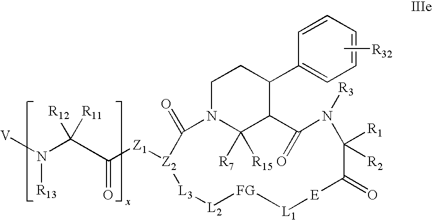

and pharmaceutically acceptable salts, enantiomers, stereoisomers, rotamers, tautomers, diastereomers, or racemates thereof;
wherein

Z₂ is nitrogen or CH;

R₃₂ is -Q¹-Q², wherein Q¹ is absent, C(O), S(O)ₚ, N(H), N(C₁₋₄-alkyl), C═N(CN), C═N(SO₂CH₃), or C═N—COH, and Q² is H, C₁₋₄-alkyl, C═N—COH—C₁₋₄-alkyl, O—-C₁₋₄-alkyl, NH₂, N(H)—C₁₋₄-alkyl, N(C₁₋₄-alkyl)₂, SO₂-aryl, SO₂—C₁₋₄-alkyl, C₃₋₆-cycloalkyl-C₀₋₄-alkyl, aryl, heteroaryl and heterocycle, each of which may be independently substituted one or more times (or preferably between one and five times) with a halogen atom, C₁₋₄-alkyl, C₁₋₄-alkyl substituted by one or more halogen atoms, or C₃₋₆-cycloalkyl.

In another embodiment, Formula IIId is represented by a compound of the Formula IIIf

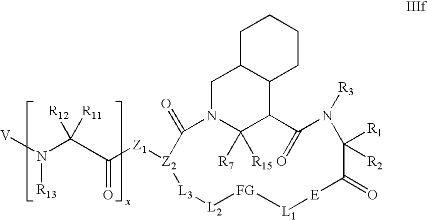

and pharmaceutically acceptable salts, enantiomers, stereoisomers, rotamers, tautomers, diastereomers, or racemates thereof.

In another embodiment, Formula IIId is represented by a compound of the Formula IIIg:

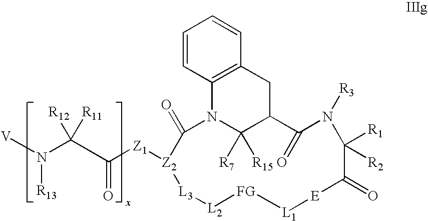

and pharmaceutically acceptable salts, enantiomers, stereoisomers, rotamers, tautomers, diastereomers, or racemates thereof.

Certain compounds of Formula III include compounds represented by Formula IIIh:

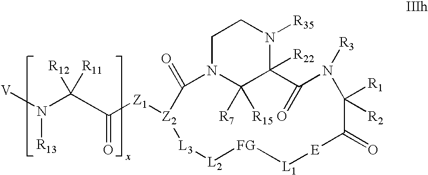

and pharmaceutically acceptable salts, enantiomers, stereoisomers, rotamers, tautomers, diastereomers, or racemates thereof;
wherein

R₃₅ is H, halogen, hydroxy, COOH, amino, carboxamide, substituted or unsubstituted-C₁₋₄-alkyl, substituted or unsubstituted C₃₋₆cycloalkylC₀-C₄alkyl, substituted or unsubstituted-C₁₋₄-alkoxy, substituted or unsubstituted C₃₋₆cycloalkylC₀-C₄alkyl-oxy-, substituted or unsubstituted arylC₀-C₄alkyl, substituted or unsubstituted heterocycleC₀-C₄alkyl, substituted or unsubstituted arylC₀-C₄alkyl-oxy and substituted or unsubstituted heterocycleC₀-C₄alkyl-oxy.

In one embodiment of Formula IIIh, R₃₅ is phenyl, optionally substituted with chloro.

Certain compounds of Formula I include compounds of Formula IV:

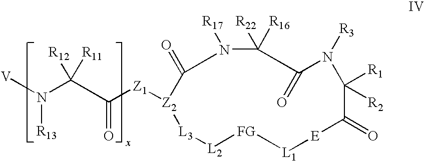

and pharmaceutically acceptable salts, enantiomers, stereoisomers, rotamers, tautomers, diastereomers, or racemates thereof.

Certain compounds of Formula IV include those compounds in which:

y is 0 or 1;

Z₂ is nitrogen or CH;

R₃ is selected from the group consisting of H and C₁₋₄-alkyl;

R₁₇ is hydrogen or is selected from the group consisting of C₁₋₄-alkyl, C₁₋₆-cycloalkyl, (CH₂)₀₋₄—C₃₋₆-cycloalkyl, aryl, alkyl-aryl and heterocycle, each of which may be independently substituted one or more times (or preferably between one and five times);

R₁₀ and R₁₁ are each, independently, selected from the group consisting of H and C₁₋₄-alkyl;

R₁₂ is selected from the group consisting of H, C₁₋₄-alkyl, C₁₋₆-cycloalkyl and aryl; and

V is selected from the group consisting of -Q¹-Q², wherein Q¹ is absent, C(O), N(H), N(C₁₋₄-alkyl), C═N(CN), C═N(SO₂CH₃), or C═N—COH, and Q² is H, C₁₋₄-alkyl, C═N—COH—C₁₋₄-alkyl, O—C₁₋₄-alkyl, NH₂, N(H)—C₁₋₄-alkyl, N(C₁₋₄-alkyl)₂, SO₂-aryl, SO₂—C₁₋₄-alkyl, C₃₋₆-cycloalkyl-C₀₋₄-alkyl, aryl, heteroaryl and heterocycle, each of which may be independently substituted one or more times (or preferably between one and five times) with a halogen atom, C₁₋₄-alkyl, C₁₋₄alkoxy, C₂-C₄alkenyloxy, C₂-C₄alkynyloxy, C₁-₄-alkyl substituted by one or more halogen atoms, or C₃₋₆-cycloalkyl;

or R₁₁ and V form the following 5-membered ring which may be further substituted:

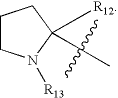

Certain other compounds of Formula IV include those compounds in which R₁₇ is selected from the group consisting of H, cyclopropylC₀-C₂alkyl, cyclopentylC₀-C₂alkyl, phenylC₁-C₂alkyl, and naphthylC₁-C₂alkyl.

Certain other compounds of Formulae I, II (including IIa and IIb), III (including IIIa through IIIh), and/or IV include those compounds in which V is selected from the group consisting of C(O)R₂₄, C(O)C(O)OR₂₄, C(O)N(H)R₂₄, C(O)C(O)N(H)R₂₄ and C(O)OR₂₄, wherein each R₂₄ is independently selected from the group consisting of H, halogen, substituted or unsubstituted-C₁₋₄-alkyl, substituted or unsubstituted C₃₋₆-cycloalkylC₀-C₄alkyl, substituted or unsubstituted arylC₀-C₄alkyl and substituted or unsubstituted heterocycleC₀-C₄alkyl, and any combination thereof.

Yet other compounds of Formulae I, II (including IIa and IIb), III (including IIIa through IIIh), and/or IV include compounds in which V is C(O)—R₂₀, wherein R₂₀ is selected from the group consisting of tert-butyl, C₃₋₆-cycloalkyl, phenyl, pyrazine, benzooxazole, 4,4-dimethyl-4,5-dihydro-oxazole, benzoimidazole, pyrimidine, thiazole, benzothiazole, benzothiazole 1,1-dioxide and quinazoline, each of which may be further independently substituted with 0-5 substitutents selected from a halogen atom, C₁₋₄-alkyl, C₁₋₄alkoxy, C₂-C₄alkenyloxy, C₂-C₄alkynyloxy, C₁₋₄-alkyl substituted by one or more halogen atoms, or C₃₋₆-cycloalkyl.

Still other compounds of Formulae I, II (including IIa and IIb), III (including IIIa through IIIh), and/or IV include compounds in which V is R₂₀ or C(O)—R₂₀, wherein R₂₀ is a residue of the formula:

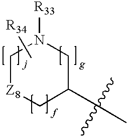

wherein

Z₈ is absent or selected from NR₃₃ or oxygen;

g and f are independently selected integers selected from the group consisting of 0, 1, 2, 3 and 4;

j is an integer selected from the group consisting of 1, 2, 3 and 4, wherein the sum of f+g+j is less than or equal to 5 and greater than or equal to 2 when Z₈ is absent and the sum of f+g+jk is less than or equal to 4 and greater than or equal to 1 when Z₈ is oxygen;

R³³ is independently selected at each occurrence from the group consisting of hydrogen, C₁₋₄alkyl, haloC₁₋₄alkyl, C₃₋₆cycloalkyl, hydroxyC₁₋₄alkyl, and C₁₋₄alkoxyC₁₋₄alkyl; and

R³⁴ represents zero to three residues each independently selected at each occurrence from the group consisting of halogen, hydroxy, amino, C₁₋₄alkyl, C₃₋₆cycloalkyl, C₁₋₄alkoxy, mono-and di-C₁₋₄alkylamino, hydroxyC₁₋₄alkyl, and C₁₋₄alkoxyC₁₋₄alkyl.

Yet other compounds of Formulae I, II (including IIa and IIb), III (including IIIa through IIIh), and/or IV include compounds in which V is C(O)—R₂₀, wherein R₂₀ is a residue of the formula:

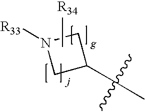

wherein

g is an integer selected from the group consisting of 0, 1, 2, 3 and 4;

j is an integer selected from the group consisting of 1, 2, 3 and 4, wherein the sum of g+j is less than or equal to 5 and greater than or equal to 2;

R³³ is independently selected at each occurrence from the group consisting of hydrogen, C₁₋₄alkyl, haloC₁₋₄alkyl, C₃₋₆cycloalkyl, hydroxyC₁₋₄alkyl, and C₁₋₄alkoxyC₁₋₄alkyl; and

R³⁴ represents zero to three residues each independently selected at each occurrence from the group consisting of halogen, hydroxy, amino, C₁₋₄alkyl, C₃₋₆cycloalkyl, C₁₋₄alkoxy, mono-and di-C₁₋₄alkylamino, hydroxyC₁₋₄alkyl, and C₁₋₄alkoxyC₁₋₄alkyl.

In another embodiment of Formula I, X is CR₅R₅ₐ, R₄ and R₅ₐ are H and R₅ is aryl-C₀₋₃-alkyl, —O-heterocycle, or heterocycle-C₀₋₃-alkyl, wherein aryl and heterocycle may be independently substituted one or more times (or preferably between one and five times) with a halogen atom, aryl, trihalomethyl, C₃₋₆-cycloalkyl or C₁₋₄-alkyl.

In yet another embodiment of Formula I, X is CR₅R₅ₐ, R₄ and R₅ₐ are H and R₅ is selected from the group consisting of piperidine, phenyl, —O-pyridinyl and CH₂-pyridinyl, wherein the phenyl and pyridinyl groups may be independently substituted one or more times (or preferably between one and five times) with a halogen atom or C₁₋₄-alkyl.

In yet another embodiment of formula I, R₅ is 5-chloro-pyridin-2-yl.

In still another embodiment of formulae I or II (including IIa and IIb), R₅ is selected from the group consisting of

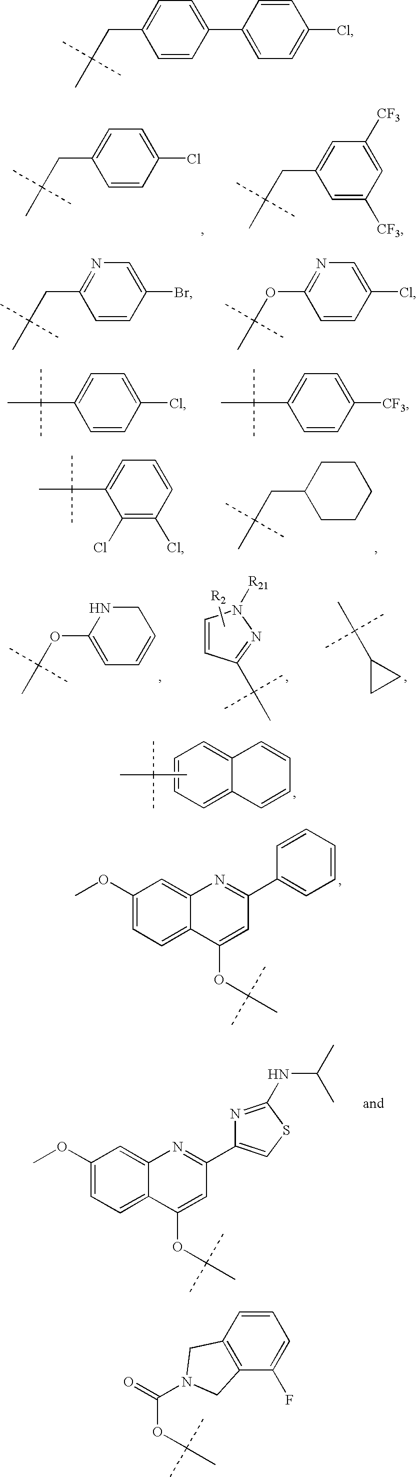

wherein R₂₁ is independently selected from the group consisting of C₁₋₄-alkyl and aryl.

In still other embodiments, CR₅R₅ₐ, taken in combination, form a spirocyclic 3 to 6 member carbocyclic ring. Certain spirocyclic rings include groups of the formula:

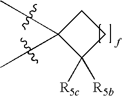

wherein
f is 0, 1, 2, 3, 4 or 5;
R_{5b} and R_{5c} are independently selected from hydrogen halogen, C₁₋₆-alkyl, C₂₋₆-alkenyl, C₂₋₆-alkynyl, C₁₋₆-alkoxide, C₃₋₇-cycloalkyl-C₀₋₄-alkyl, phenyl-C₀₋₄-alkyl, naphthyl-C₀₋₄-alkyl, heteroaryl-C₀₋₄-alkyl, or two substitutents taken together form a fused or spirocyclic 3 to 7 membered carbocyclic ring, each of which is substituted with 0-3 independently selected halogen atoms or C₁₋₄-alkyl groups.

In yet another embodiment of Formula I, R₂ is selected from the group consisting of propyl and (CH₂)₂-cyclobutyl.

In still another embodiment of Formula I, R₁₁ is H and R₁₂ is C₃₋₆-cycloalkyl.

In one embodiment of Formula I, R₁₂ is cyclohexyl.

In another embodiment of formula I, V is selected from the group consisting of C(O)—N(H)-t-butyl.

Yet other compounds of any one of Formulae I, II (including IIa and IIb), III (including IIIa through IIIh), and/or IV include compounds in which V is C(O)—N(H)-t-butyl or C(O)—R₂₀, wherein R₂₀ is selected from the group consisting of C₃₋₆-cycloalkyl, phenyl, pyrazine, benzooxazole, 4,4-dimethyl-4,5-dihydro-oxazole, benzoimidazole, pyrimidine, thiazole, benzothiazole, benzothiazole 1,1-dioxide and quinazoline, all of which may be further independently substituted with a halogen atom, CF₃, C₁₋₄-alkyl, C₁₋₄alkoxy, C₂-C₄alkenyloxy, C₂-C₄alkynyloxy, or C₃₋₆-cycloalkyl.

In certain other compounds of any one of Formulae I, H (including IIa and IIb), III (including IIIa through IIIh), and/or IV, V is selected from the group consisting of C₃₋₆-cycloalkyl, phenyl, pyrazine, benzooxazole, 4,4-dimethyl-4,5-dihydro-oxazole, benzoimidazole, pyrimidine, thiazole, benzothiazole, benzothiazole 1,1-dioxide and quinazoline, all of which may be further independently substituted with a halogen atom, CF₃, C₁₋₄-alkyl , C₁₋₄alkoxy, C₂-C₄alkenyloxy, C₂-C₄alkynyloxy, or C₃₋₆-cycloalkyl.

In yet another embodiment of Formulae I, II (including IIa and IIb), III (including IIIa through IIIh), and/or IV, V is R₂₀ or C(O)—R₂₀, wherein R₂₀ is selected from the group consisting of C₃₋₆-cycloalkyl, phenyl, pyrazine, benzooxazole, 4,4-dimethyl-4,5-dihydro-oxazole, benzoimidazole, pyrimidine, benzothiazole 1,1-dioxide and quinazoline, all of which may be further independently substituted with a halogen atom, CF₃, C₁₋₄-alkyl or C₃₋₆-cycloalkyl.

In still another embodiment of Formulae I, II (including IIa and IIb), III (including IIIa through IIIh), and/or IV, V is R₂₀ or C(O)—R₂₀, wherein R₂₀ is selected from the group consisting of

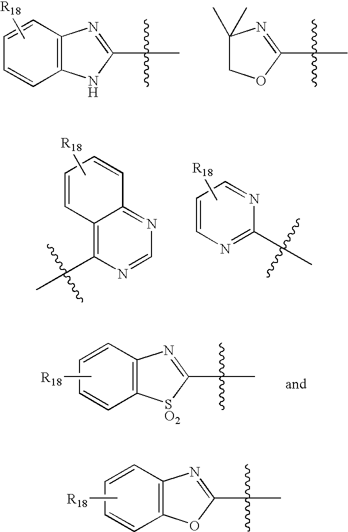

wherein R₁₈ is selected from the group consisting of hydrogen, a halogen atom, aryl, C₁₋₄-alkyl, C₁₋₄alkoxy, C₂-C₄alkenyloxy, C₂-C₄alkynyloxy, C₁₋₄-alkyl substituted by one or more halogen atoms, or C₃₋₆-cycloalkyl.

In one embodiment of Formulae I, II (including IIa and IIb), III (including IIIa through IIIh), and/or IV, V is R₂₀ or C(O)—R₂₀, wherein R₂₀ is selected from the group consisting of

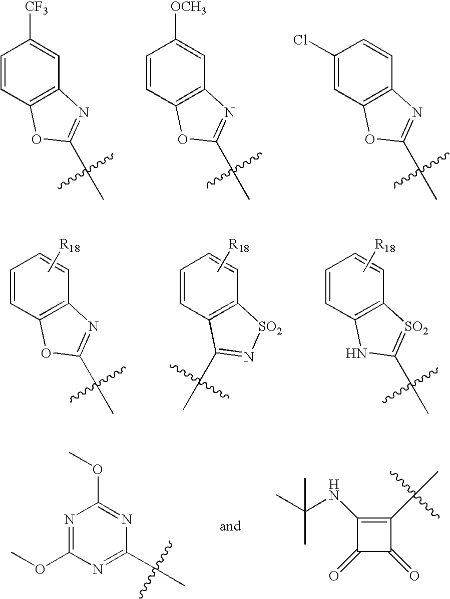

wherein R₁₈ is selected from the group consisting of hydrogen, a halogen atom, aryl, C₁₋₄-alkyl, C₁₋₄alkoxy, C₂-C₄alkenyloxy, C₂-C₄alkynyloxy, C₁₋₄-alkyl substituted by one or more halogen atoms, or C₃₋₆-cycloalkyl.

In another embodiment of Formulae I, II (including IIa and IIb), III (including IIIa through IIIh), and/or IV, V is selected from the group consisting of C₃₋₆-cycloalkyl, phenyl, pyrazine, benzooxazole, 4,4-dimethyl-4,5-dihydro-oxazole, benzoimidazole, pyrimidine, thiazole, benzothiazole, benzothiazole 1,1-dioxide and quinazoline, all of which may be further independently substituted with a halogen atom, C₁₋₄-alkyl, C₁₋₄alkoxy, C₂-C₄alkenyloxy, C₂-C₄alkynyloxy, C₁₋₄-alkyl substituted by one or more halogen atoms, or C₃₋₆-cycloalkyl.

In yet another embodiment of Formula I, II (including IIa and IIb), III (including IIIa through IIIh), and/or IV, variable V is selected from the group consisting of R²⁰ and C(O)—R²⁰, wherein R²⁰ is selected from the group consisting of C₃₋₆-cycloalkyl, mono- and di-C₁₋₄alkylamino, phenyl, pyrazine, benzooxazole, 4,4-dimethyl-4,5-dihydro-oxazole, benzoimidazole, pyrimidine, benzothiazole 1,1-dioxide and quinazoline, each of which may be further independently substituted with a halogen atom, CF₃, C₁₋₄alkoxy, C₂-C₄alkenyloxy, C₂-C₄alkynyloxy, or C₃₋₆-cycloalkyl.

In still another embodiment of Formula. I, II (including IIa and IIb), III (including IIIa through IIIh), and/or IV, variable V is selected from the group consisting of R²⁰ and C(O)—R²⁰, wherein R²⁰ is selected from the group consisting of

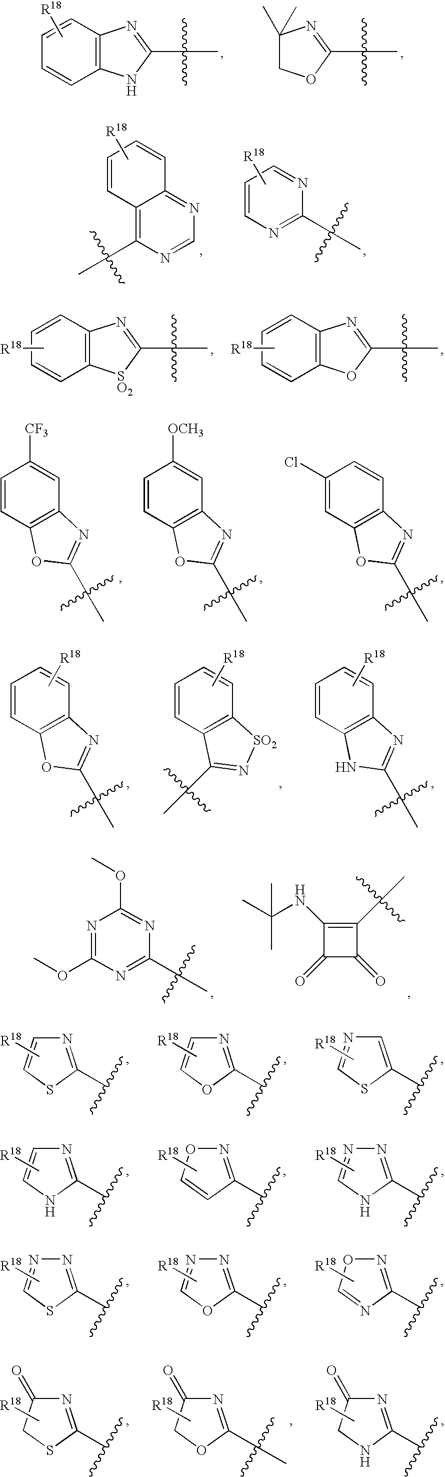

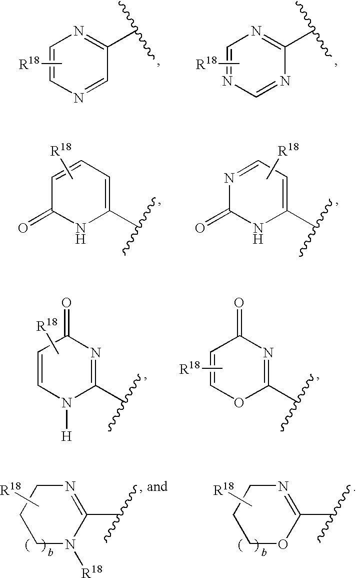

wherein b is 0, 1, or 2; and R₁₈ is selected from the group consisting of hydrogen, a halogen atom, aryl, trihalomethyl, and C₁₋₄-alkyl.

In one embodiment, any of the C₃₋₆-cycloalkyl groups of Formula I, or any subformula thereof, may be independently substituted one or more times (or preferably between one and five times) with a halogen atom, aryl, heteroaryl, trihalomethyl, C₁₋₄-alkoxy or C₁₋₄-alkyl.

In one embodiment of Formula I, or any subformulae thereof, any of the heterocycle groups are independently selected from the group consisting of acridinyl, carbazolyl, cinnolinyl, quinoxalinyl, pyrrazolyl, indolyl, benzotriazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetrahydroquinoline, benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrazolyl, tetrazolopyridyl, thiadiazolyl, thiazolyl, thienyl, triazolyl, azetidinyl, 1,4-dioxanyl, hexahydroazepinyl, piperazinyl, piperidinyl, pyridin-2-onyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl, and N-oxides thereof, all of which may be independently further substituted one or more times (or preferably between one and five times) with a halogen atom, C₁₋₄-alkyl, C₁₋₄-alkyl substituted by one or more halogen atoms, or C₃₋₆-cycloalkyl.

Preferred embodiments of the compounds of the invention (including pharmaceutically acceptable salts thereof, as well as enantiomers, stereoisomers, rotamers, tautomers, diastereomers, or racemates thereof) are shown below in Table A and Table B, and are also considered to be “compounds of the invention.”

TABLE A

| Structure | Compound No. |
|---|---|
| 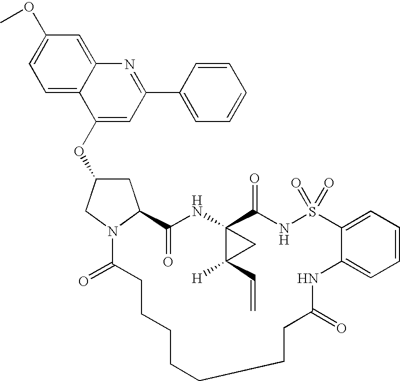 | A-1 |
| 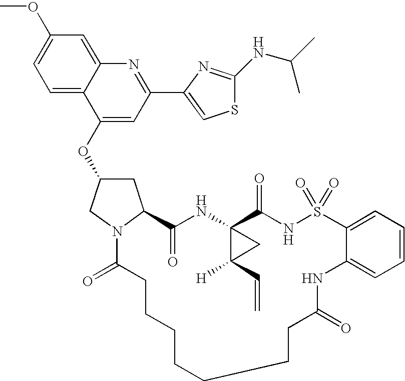 | A-2 |
| 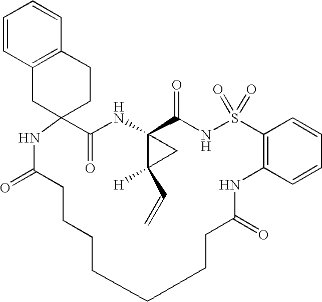 | A-3 |
| 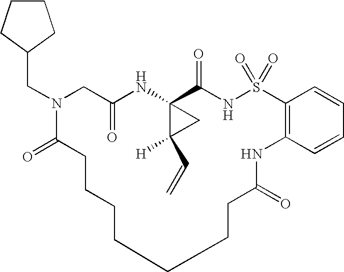 | A-4 |
| 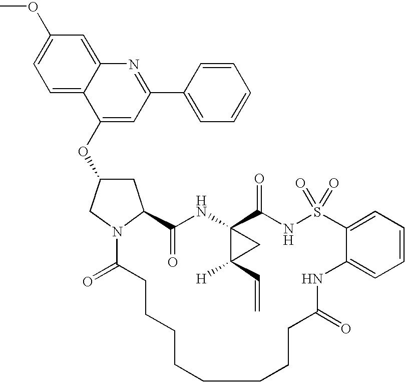 | A-5 |
| 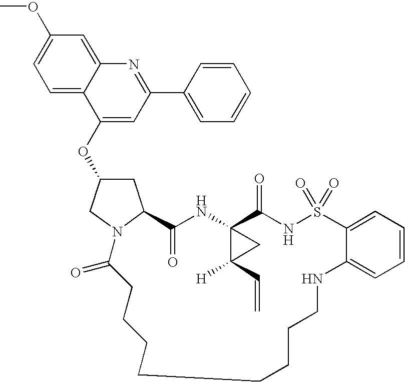 | A-6 |
| 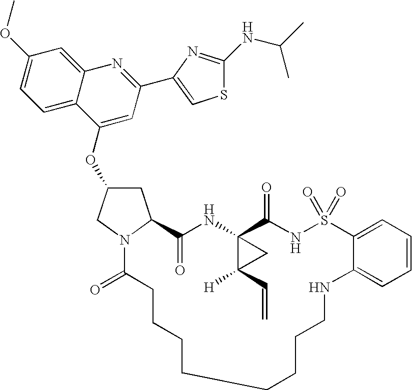 | A-7 |
| 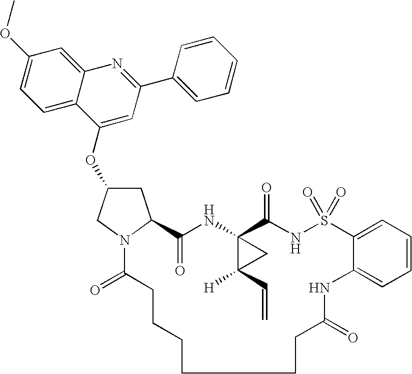 | A-8 |
| 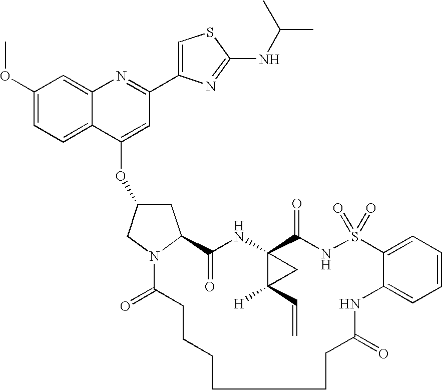 | A-9 |
| 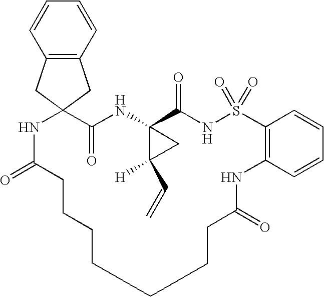 | A-10 |
| 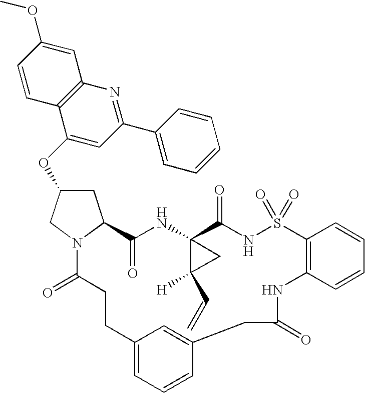 | A-11 |
| 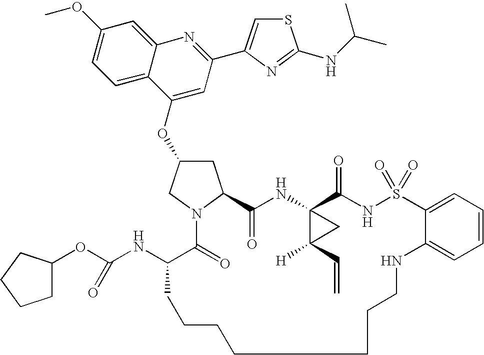 | A-12 |
| 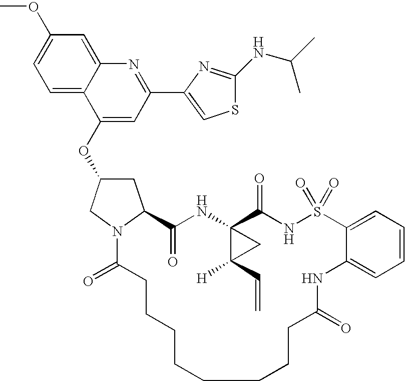 | A-13 |
| 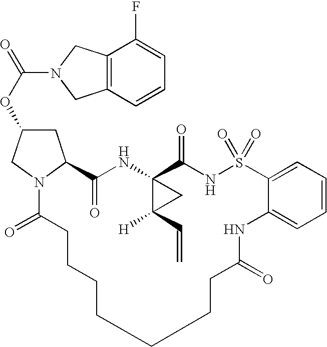 | A-14 |
| 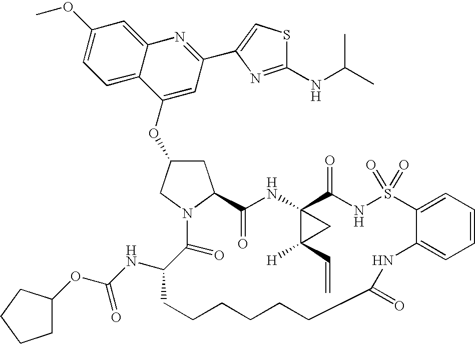 | A-15 |
| 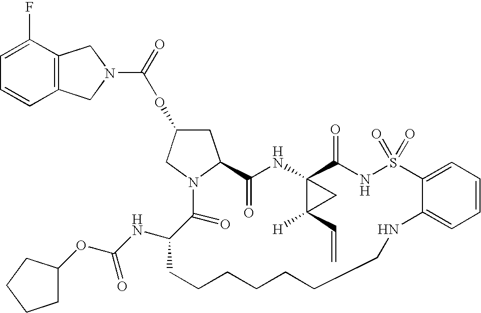 | A-16 |
| 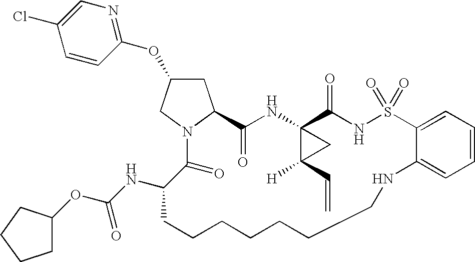 | A-17 |
| 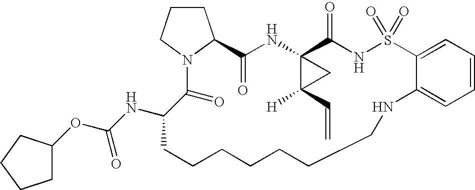 | A-18 |
| 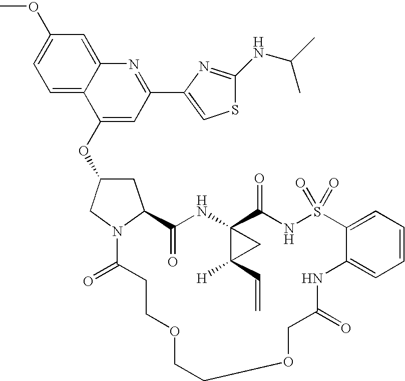 | A-19 |
| 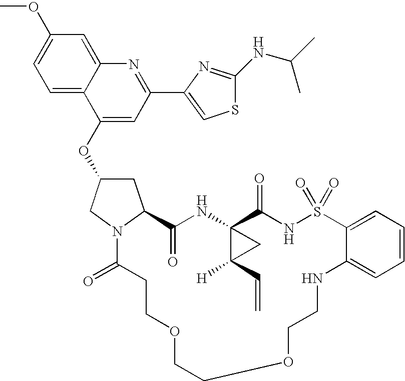 | A-20 |
| 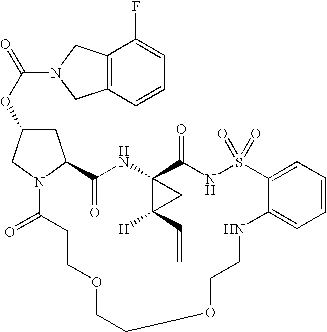 | A-21 |
| 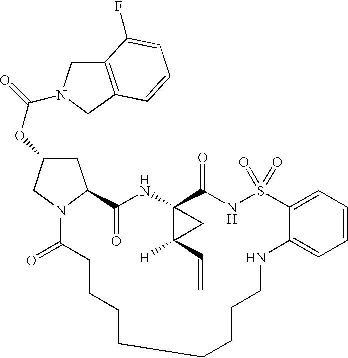 | A-22 |
| 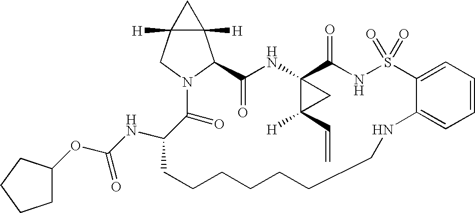 | A-23 |
| 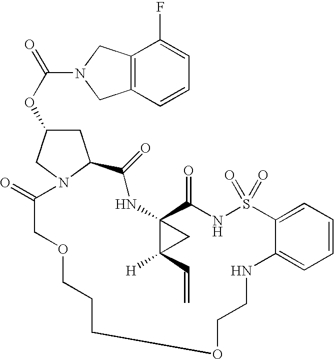 | A-24 |
| 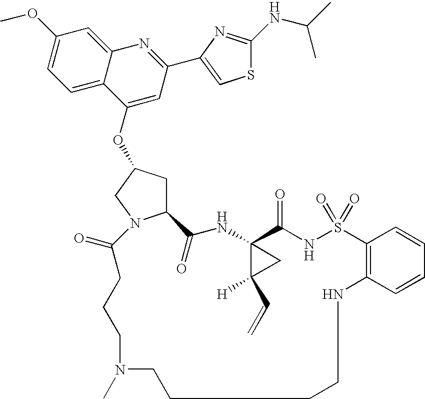 | A-25 |
| 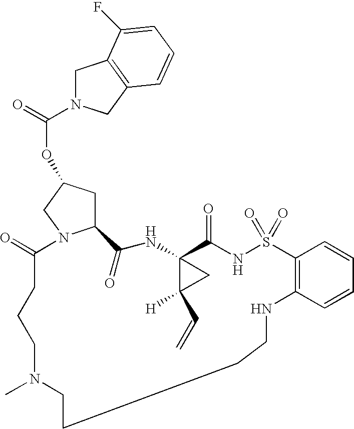 | A-26 |
| 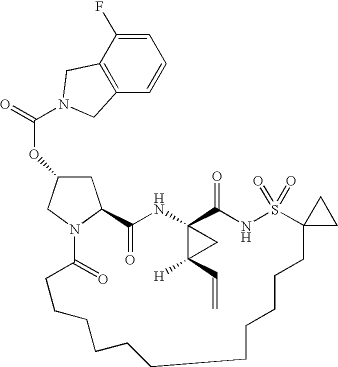 | A-27 |
| 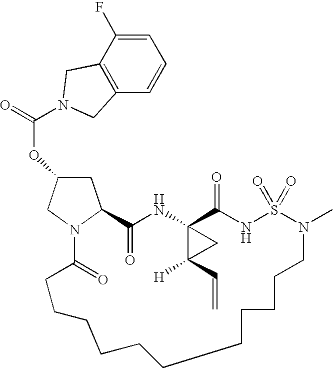 | A-28 |
| 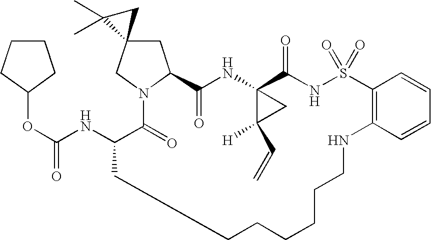 | A-29 |
| 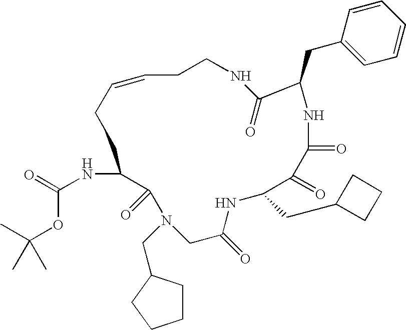 | A-30 |
| 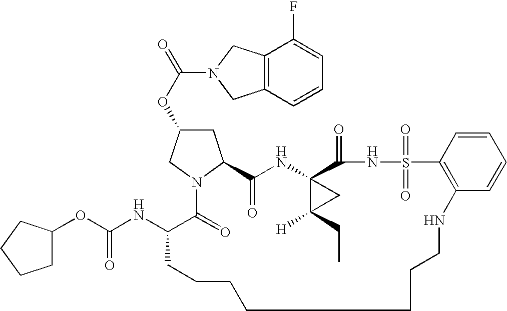 | A-31 |
| 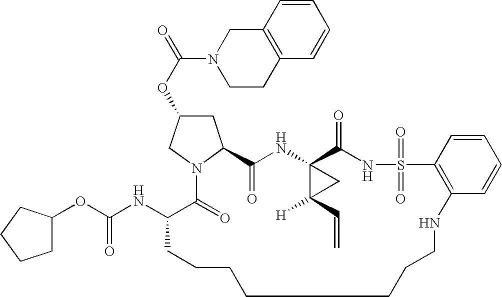 | A-32 |
| 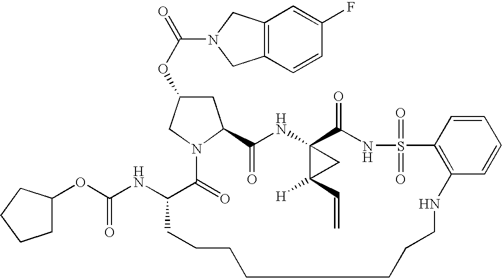 | A-33 |
| 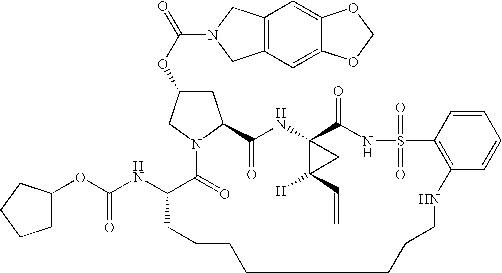 | A-34 |
| 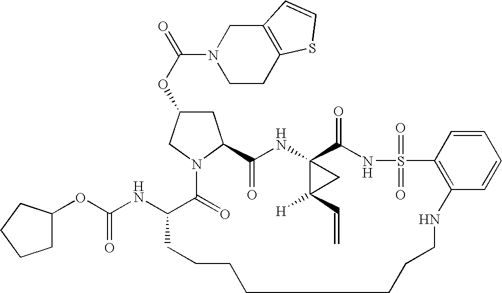 | A-35 |
| 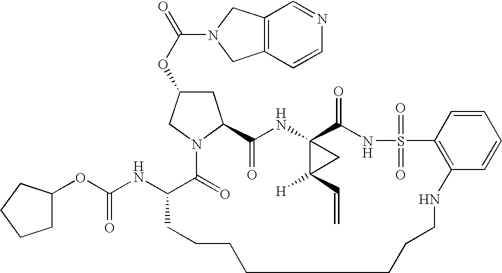 | A-36 |
| 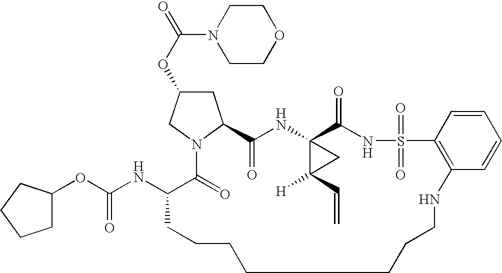 | A-37 |
| 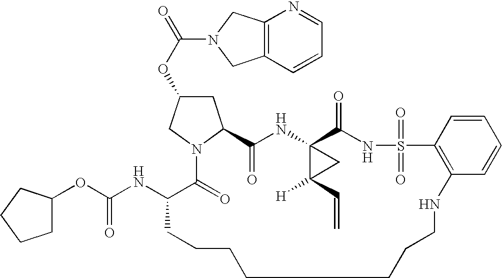 | A-38 |
| 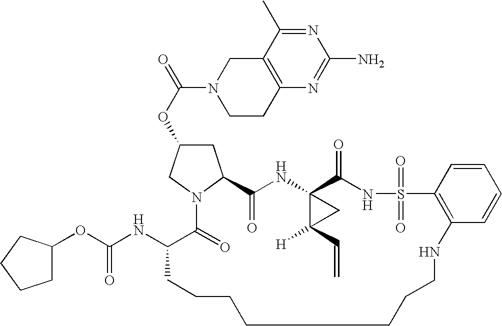 | A-39 |
| 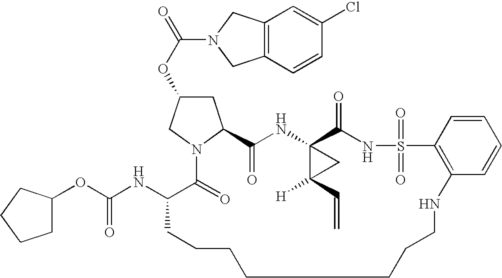 | A-40 |
| 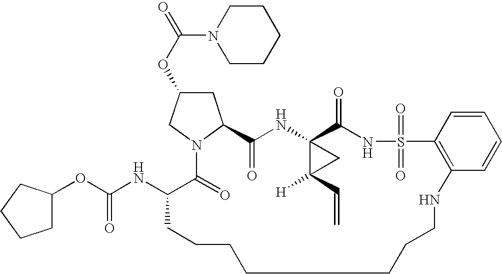 | A-41 |
| 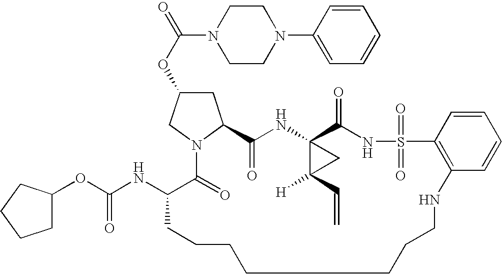 | A-42 |
| 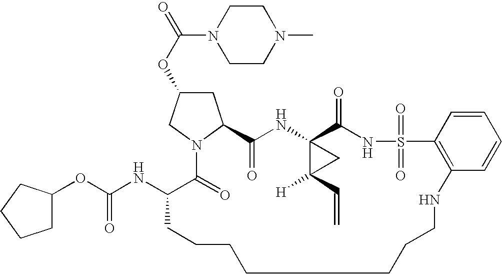 | A-43 |
| 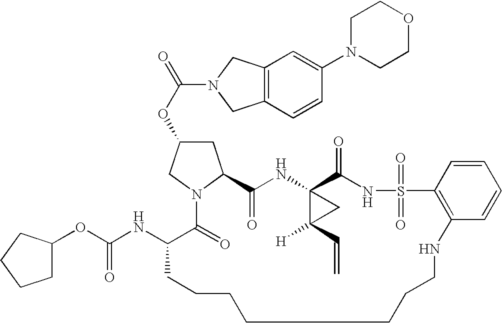 | A-44 |
| 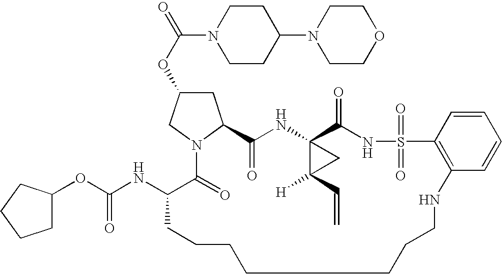 | A-45 |
| 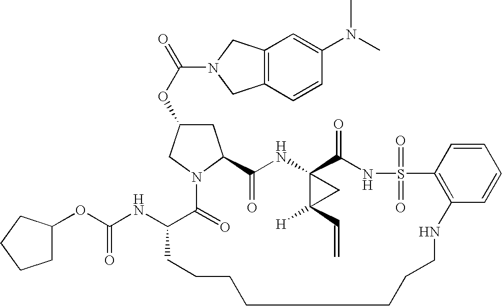 | A-46 |
| 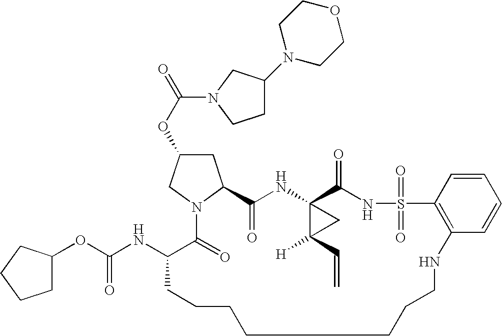 | A-47 |
| 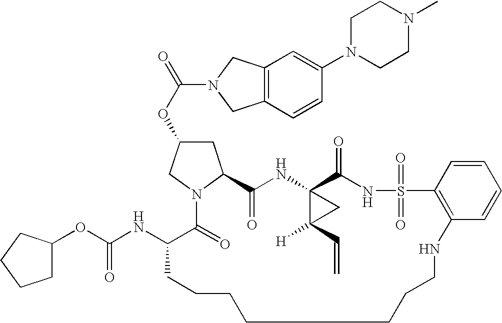 | A-48 |
| 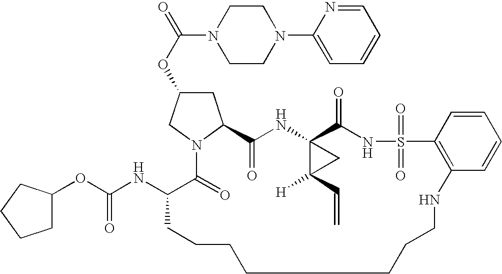 | A-49 |
| 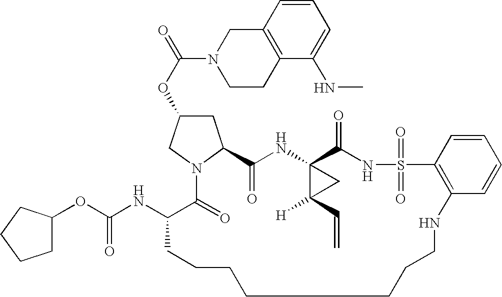 | A-50 |
| 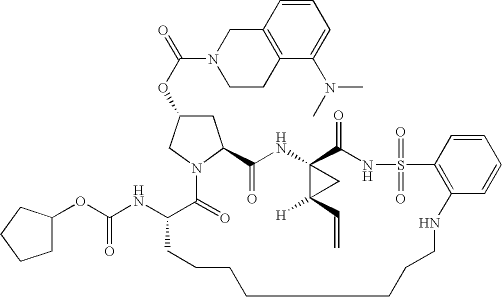 | A-51 |
| 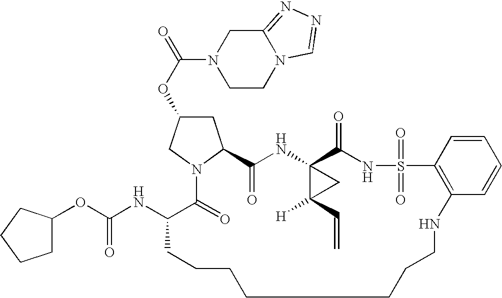 | A-52 |
| 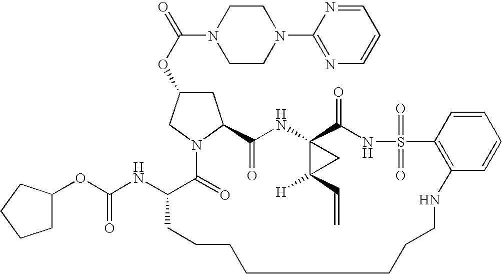 | A-53 |
| 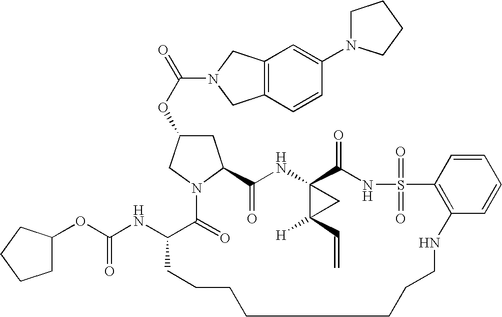 | A-54 |
| 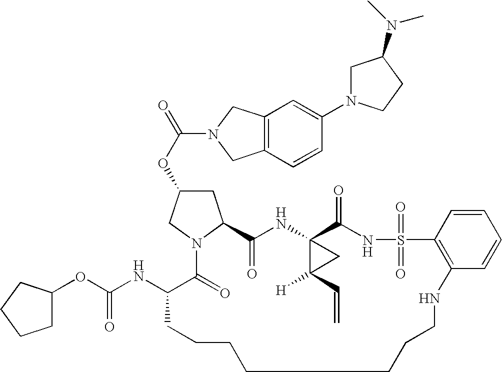 | A-55 |
| 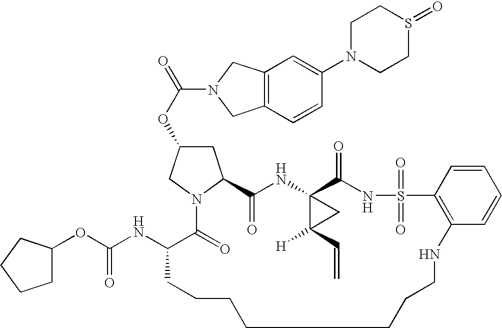 | A-56 |
| 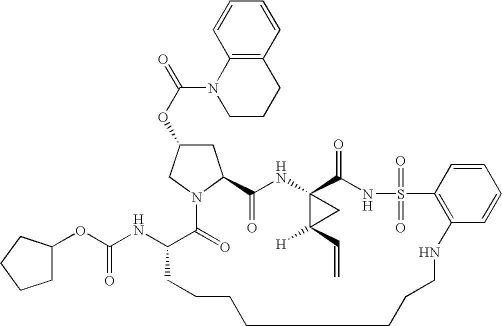 | A-57 |
| 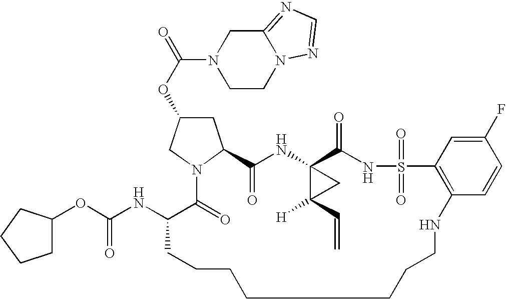 | A-58 |
| 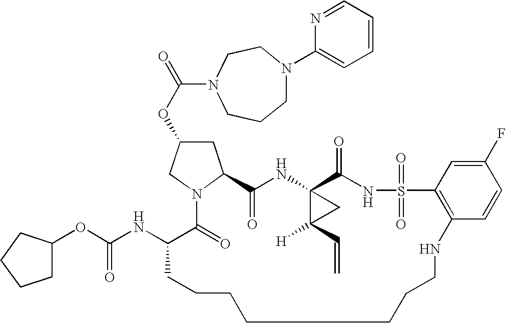 | A-59 |
| 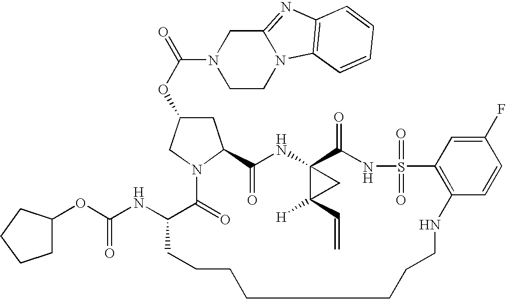 | A-60 |
| 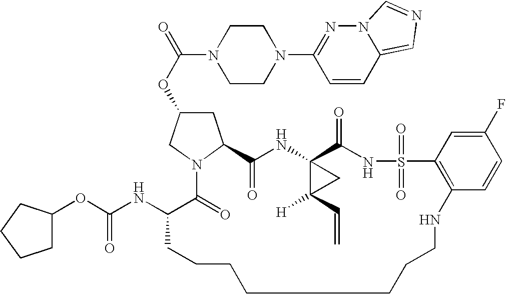 | A-61 |
| 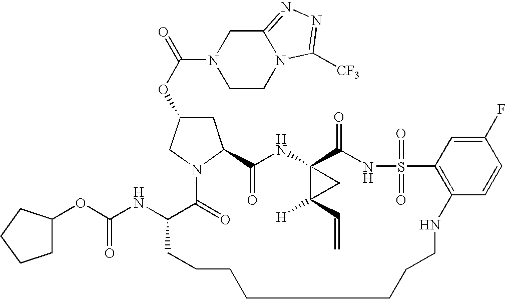 | A-62 |
| 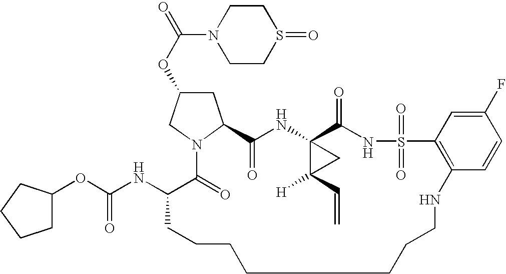 | A-63 |
| 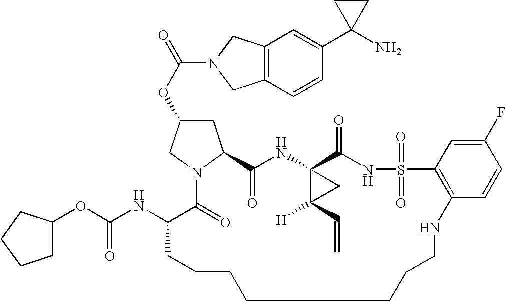 | A-64 |
| 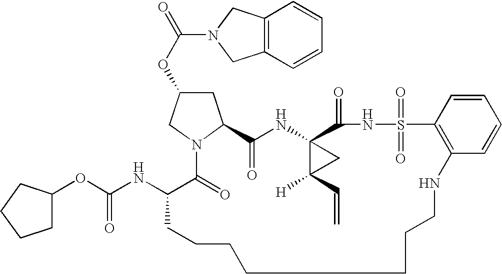 | A-65 |
| 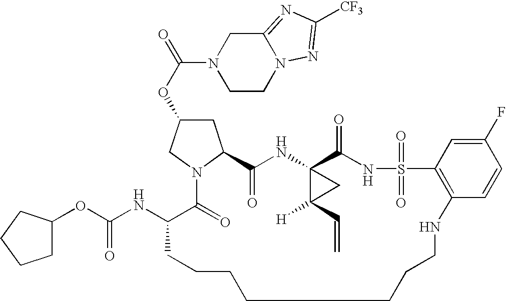 | A-66 |
| 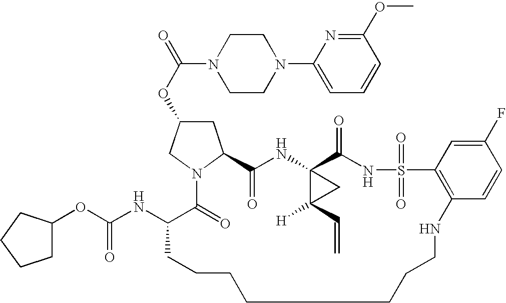 | A-67 |
| 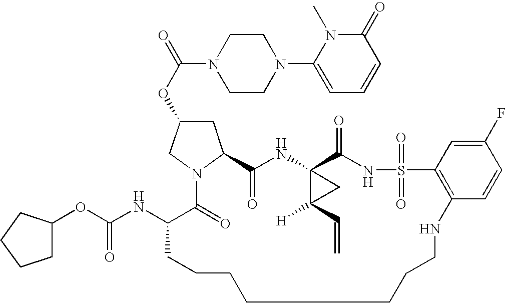 | A-68 |
| 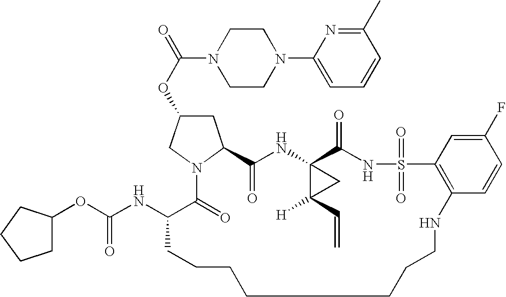 | A-69 |
| 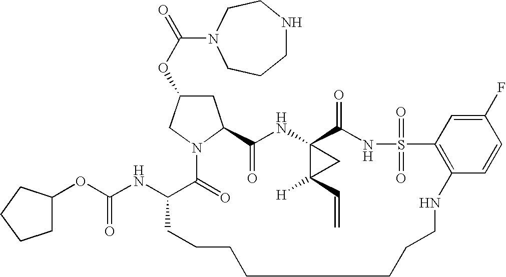 | A-70 |
| 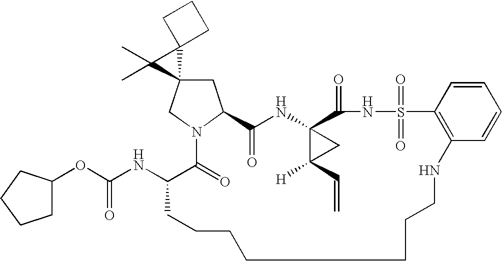 | A-71 |
| 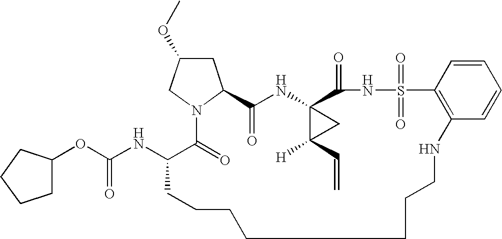 | A-72 |
| 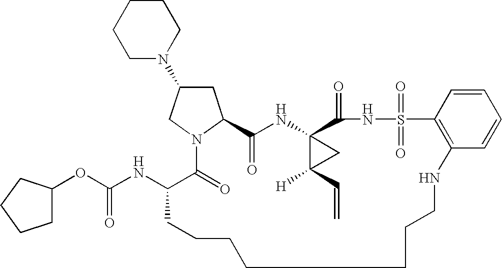 | A-73 |
| 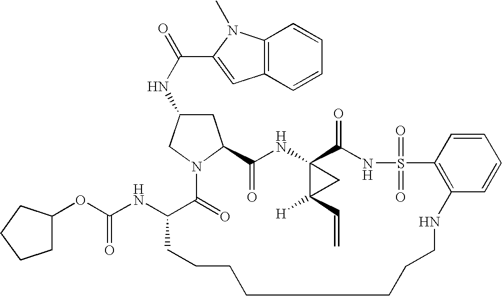 | A-74 |
| 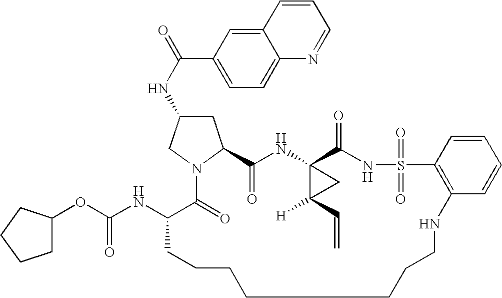 | A-75 |
| 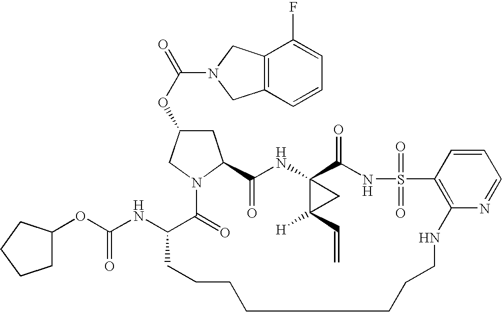 | A-76 |
| 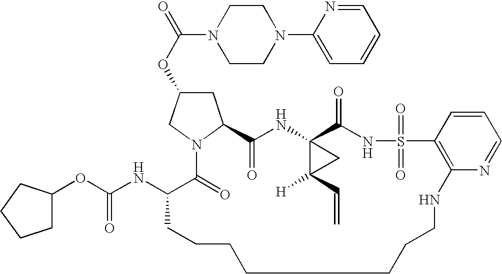 | A-77 |
| 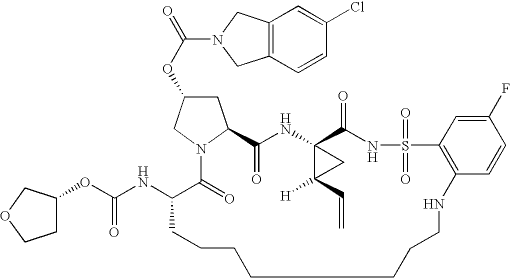 | A-78 |
| 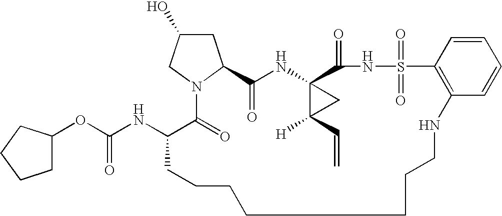 | A-79 |
| 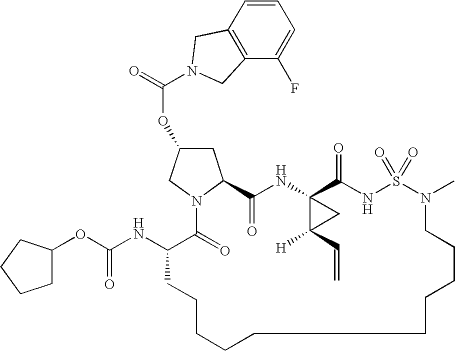 | A-80 |
| 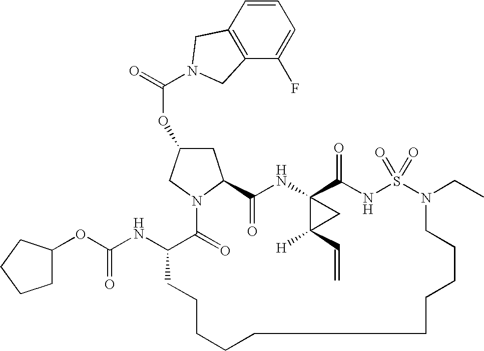 | A-81 |
| 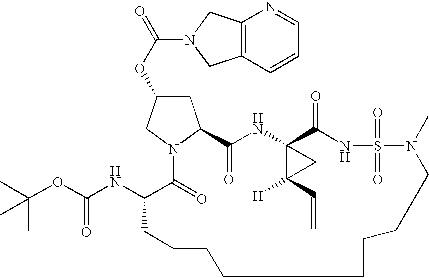 | A-82 |
| 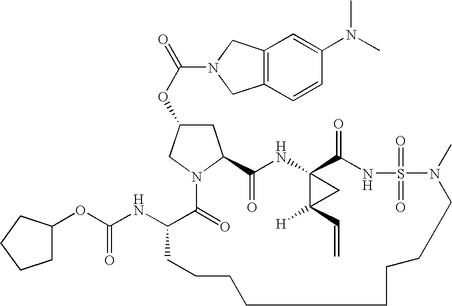 | A-83 |
| 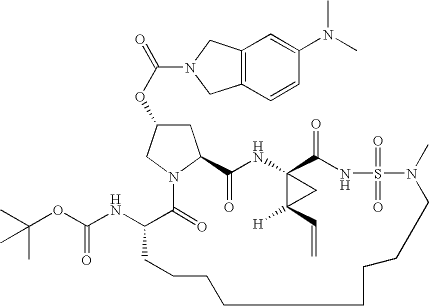 | A-84 |
| 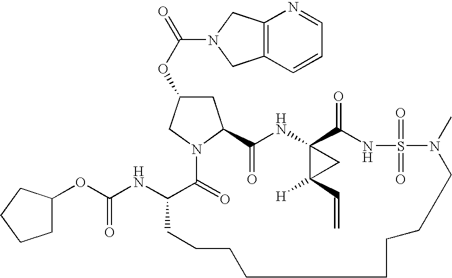 | A-85 |
| 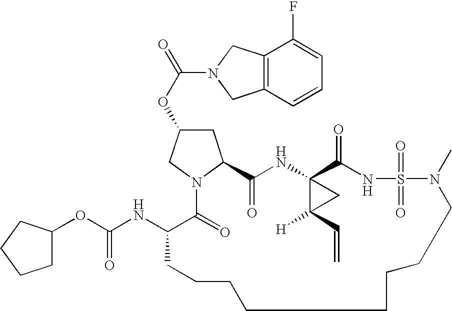 | A-86 |
| 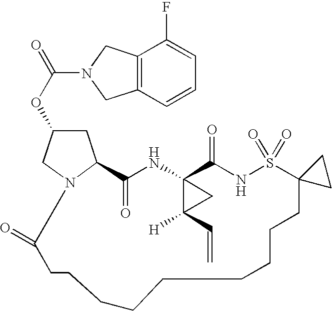 | A-87 |
| 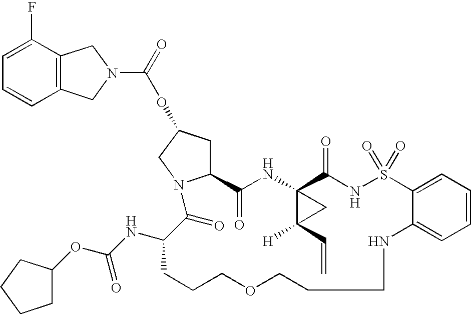 | A-88 |
| 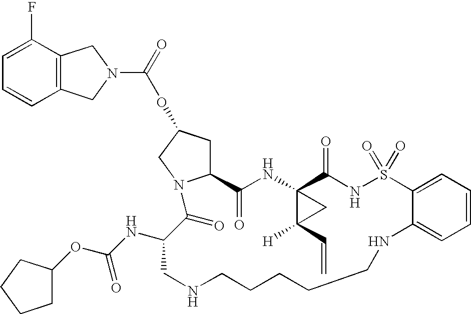 | A-89 |
| 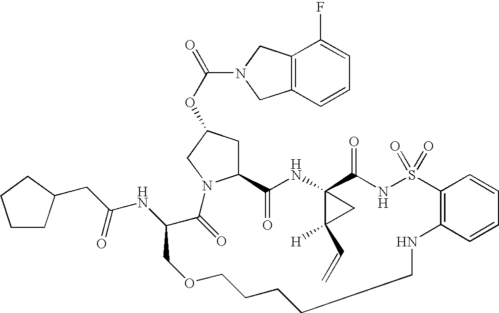 | A-90 |
| 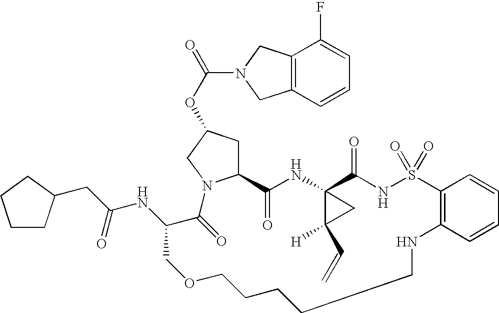 | A-91 |
| 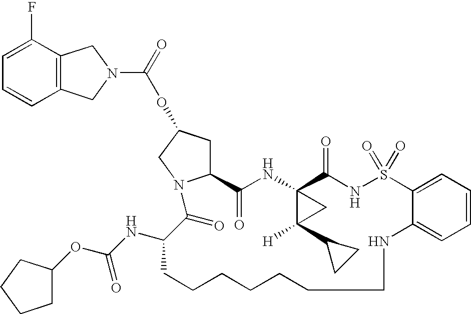 | A-92 |
| 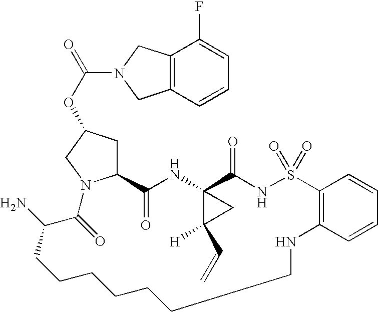 | A-93 |
| 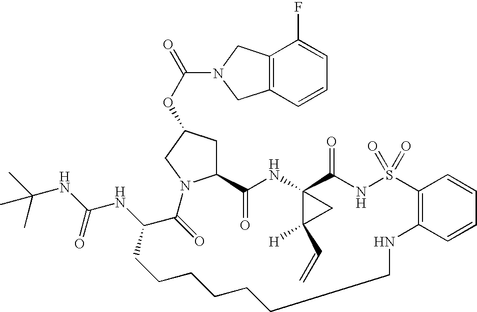 | A-94 |
| 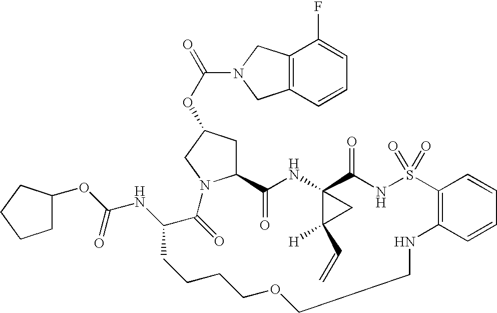 | A-95 |
| 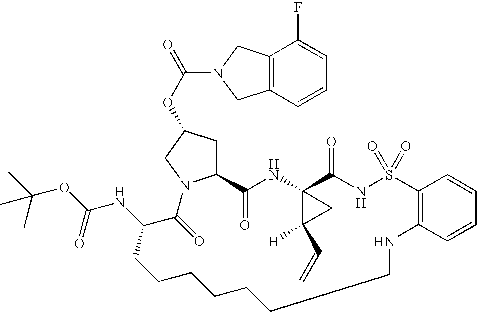 | A-96 |
| 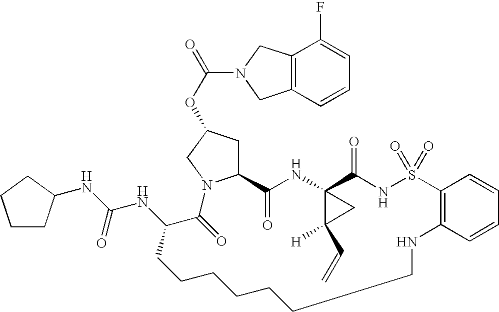 | A-97 |
| 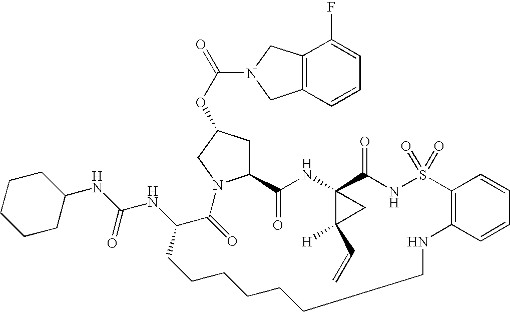 | A-98 |
| 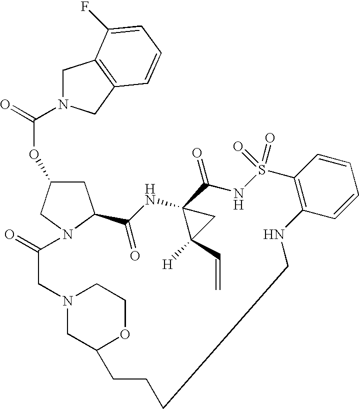 | A-99 |
| 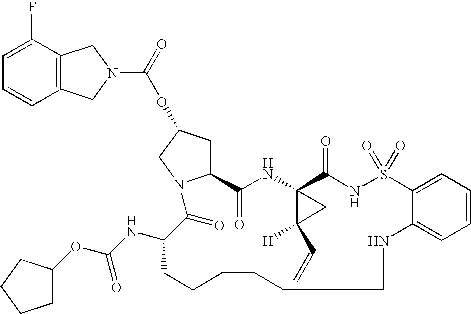 | A-100 |
| 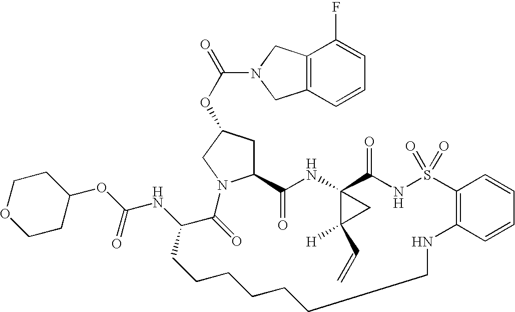 | A-101 |
| 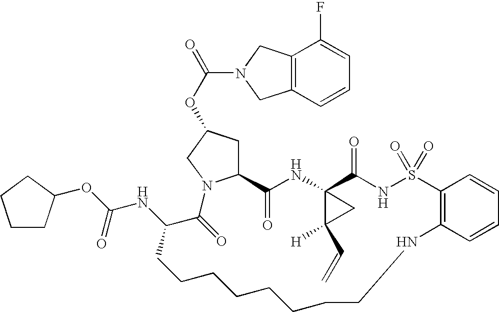 | A-102 |
| 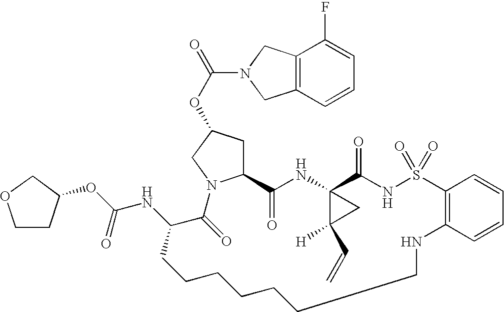 | A-103 |
| 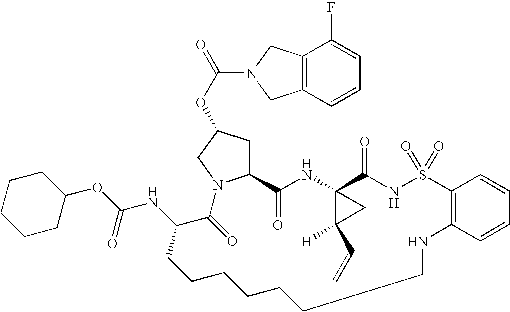 | A-104 |
| 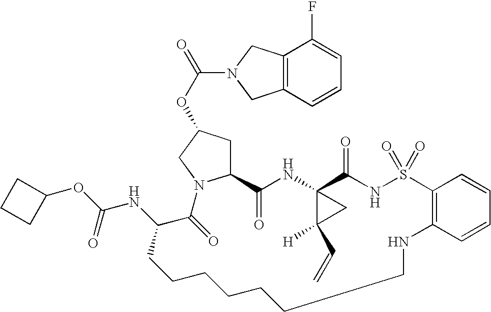 | A-105 |
| 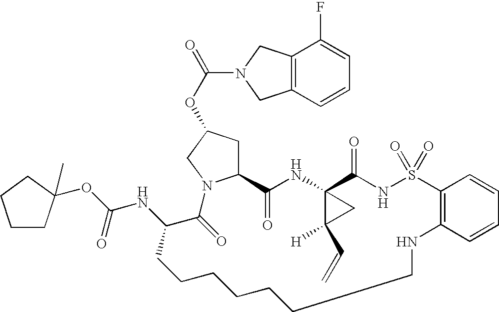 | A-106 |
| 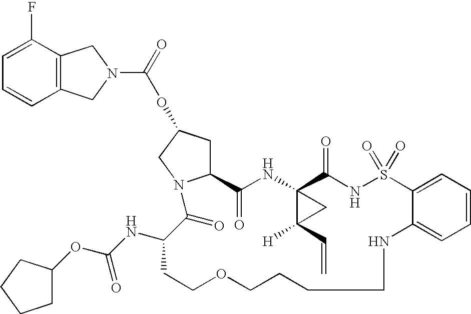 | A-107 |
| 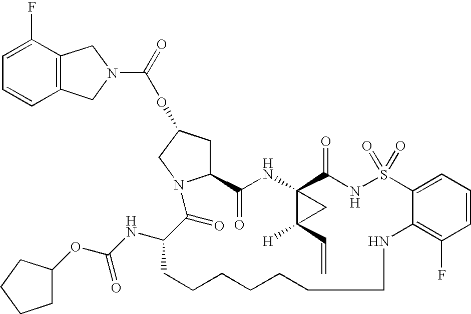 | A-108 |
| 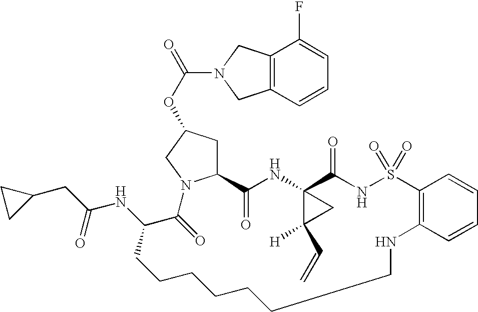 | A-109 |
| 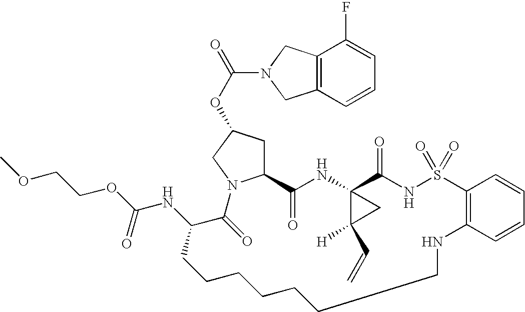 | A-110 |
| 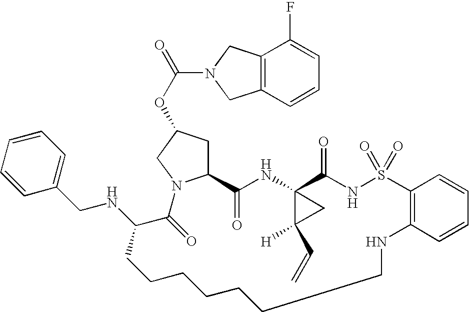 | A-111 |
| 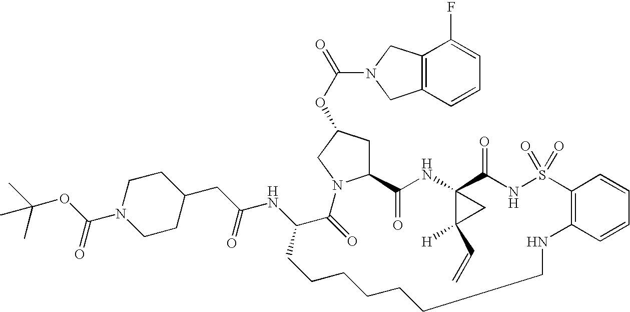 | A-112 |
| 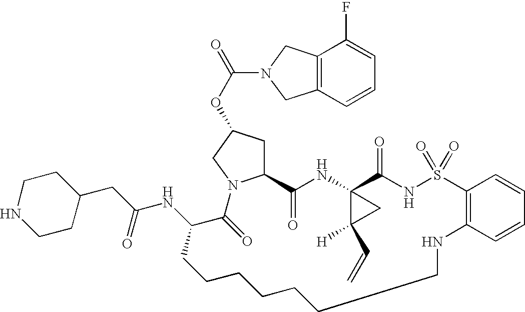 | A-113 |
| 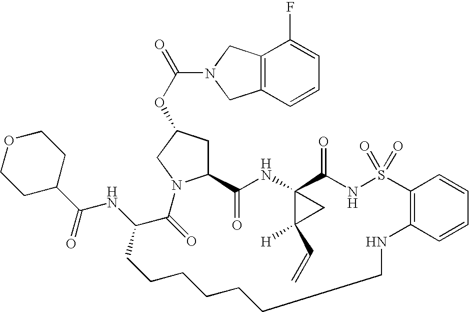 | A-114 |
| 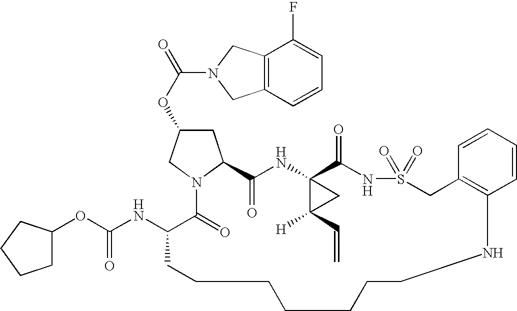 | A-115 |
| 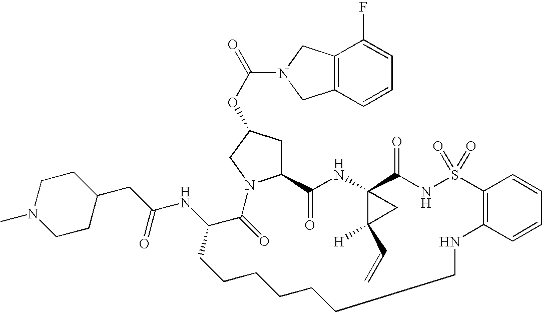 | A-116 |
| 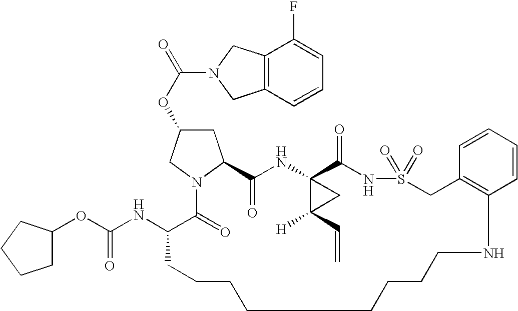 | A-117 |
| 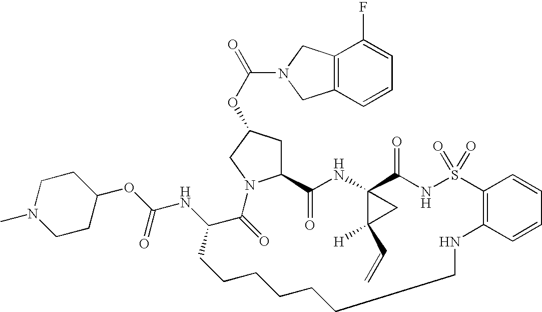 | A-118 |
| 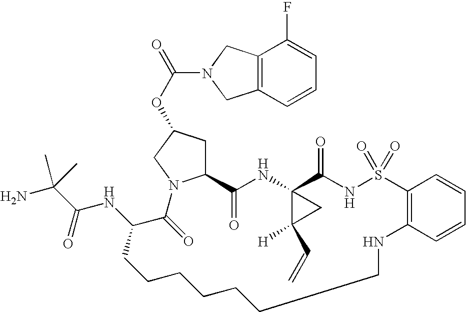 | A-119 |
| 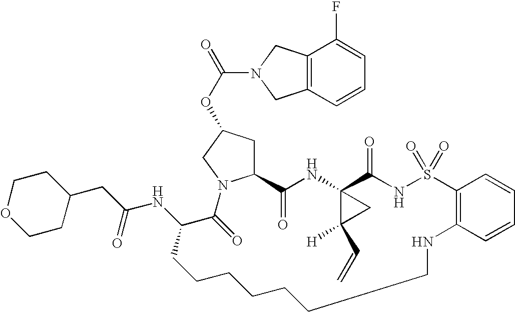 | A-120 |
| 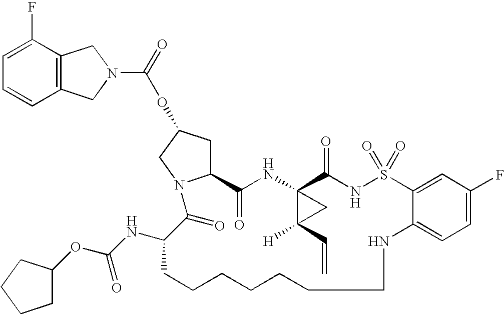 | A-121 |
| 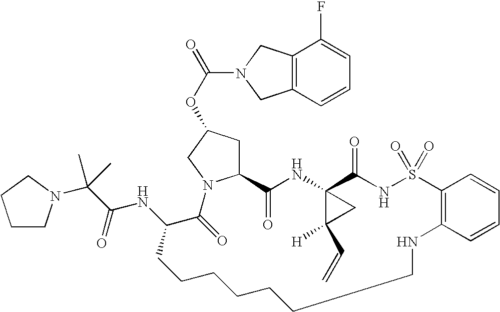 | A-122 |
| 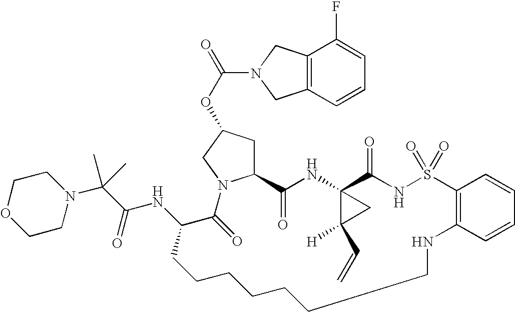 | A-123 |
| 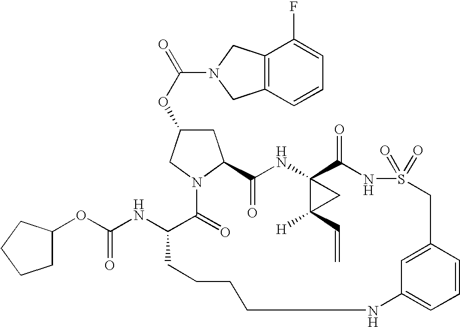 | A-124 |
| 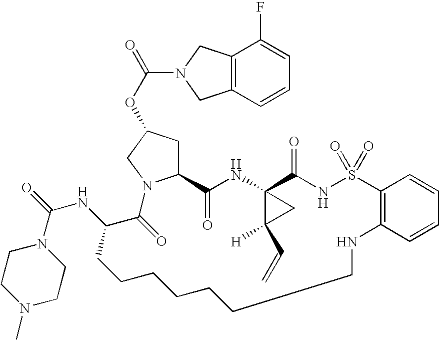 | A-125 |
| 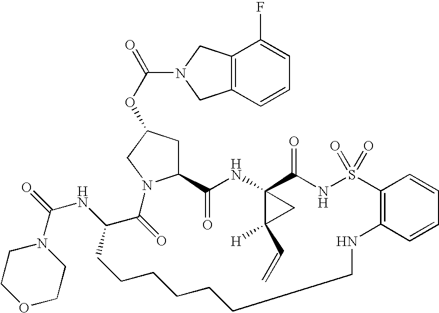 | A-126 |
| 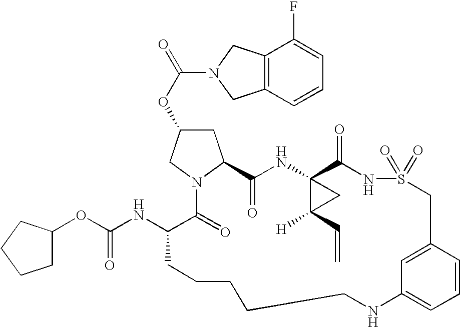 | A-127 |
| 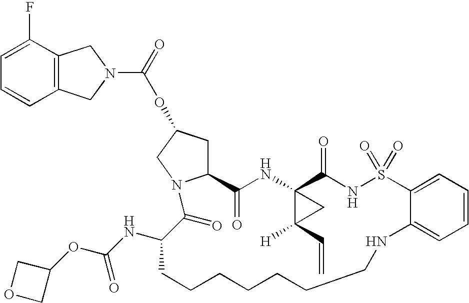 | A-128 |
| 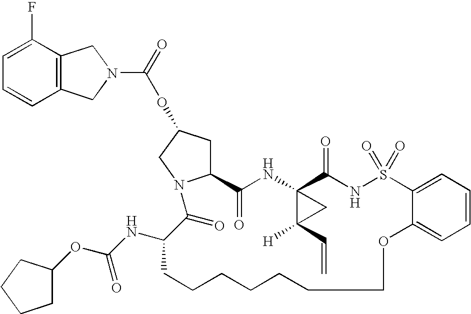 | A-129 |
| 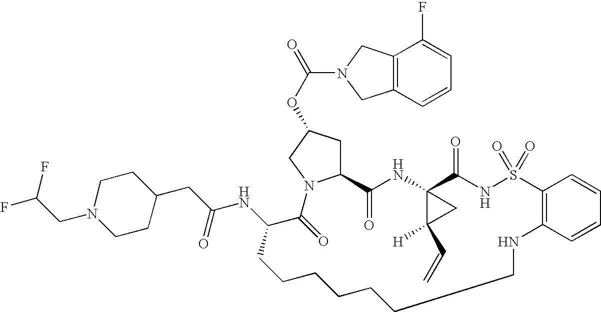 | A-130 |
| 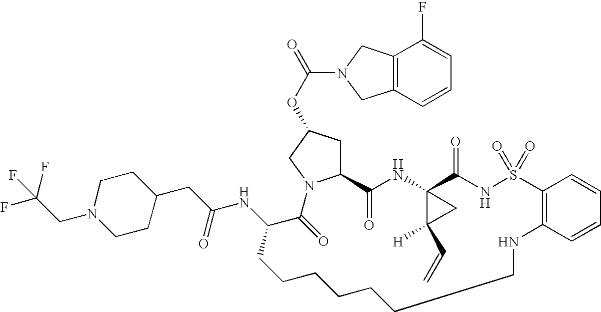 | A-131 |
| 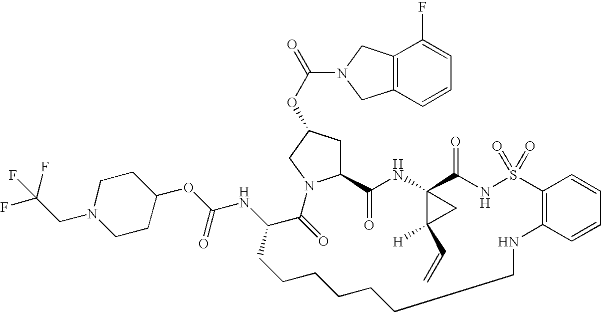 | A-132 |
| 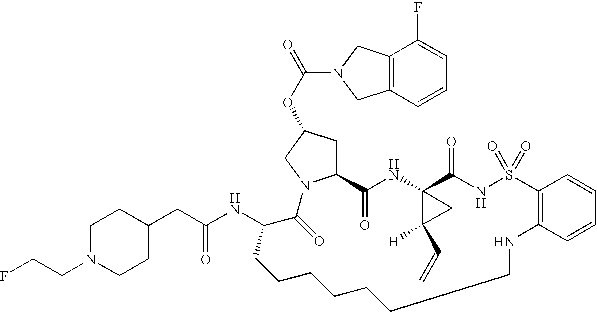 | A-133 |
| 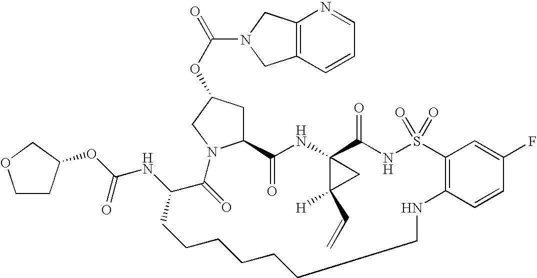 | A-134 |
| 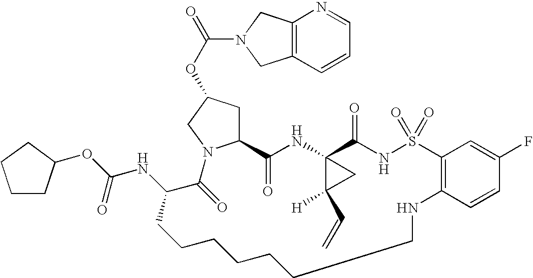 | A-135 |
| 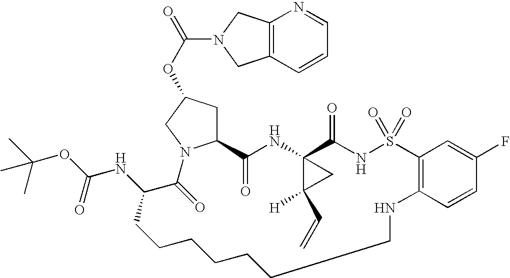 | A-136 |
| 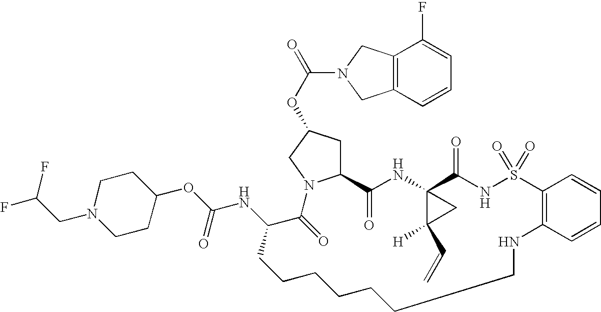 | A-137 |
| 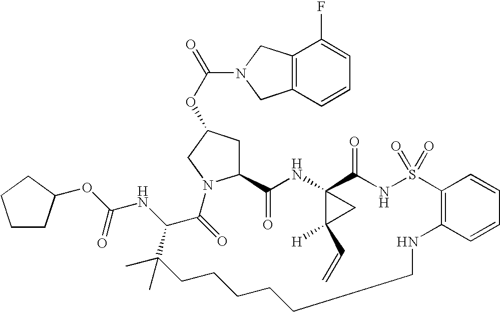 | A-138 |
| 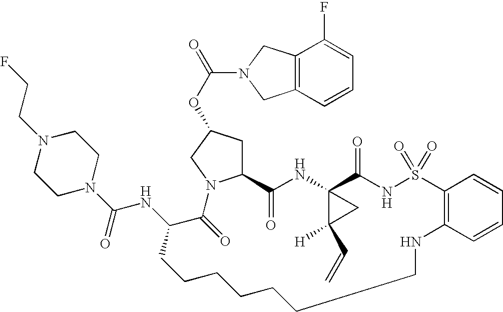 | A-139 |
| 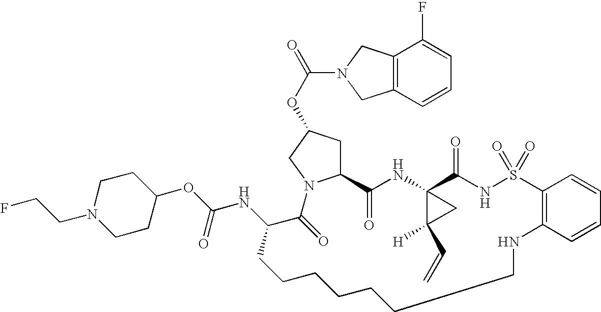 | A-140 |
| 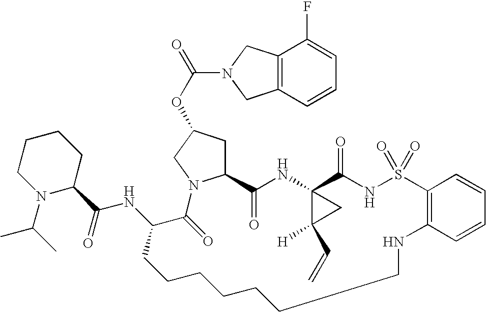 | A-141 |
| 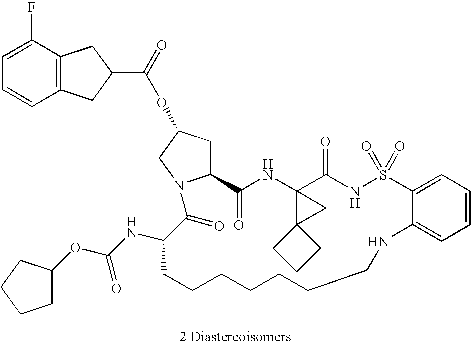 | A-142 |
| 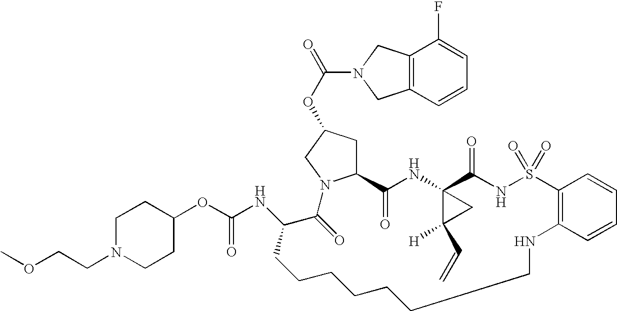 | A-143 |
| 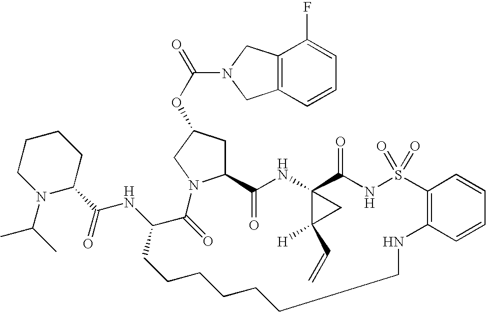 | A-144 |
| 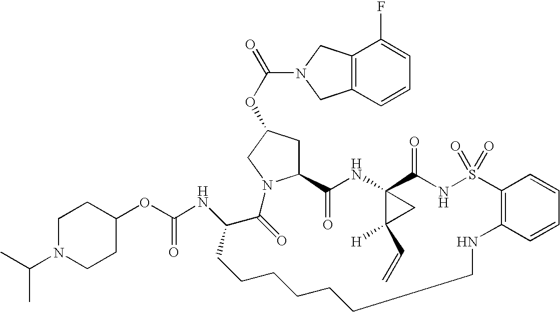 | A-145 |
| 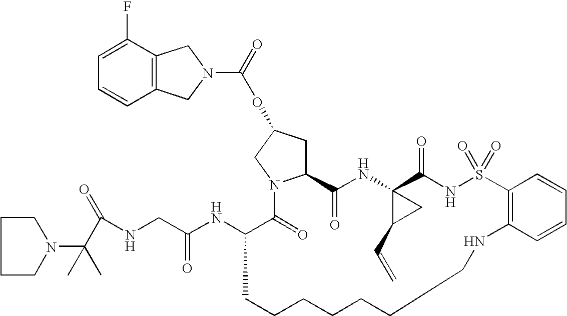 | A-146 |
| 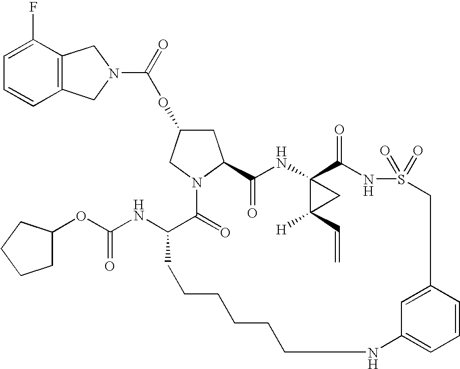 | A-147 |
| 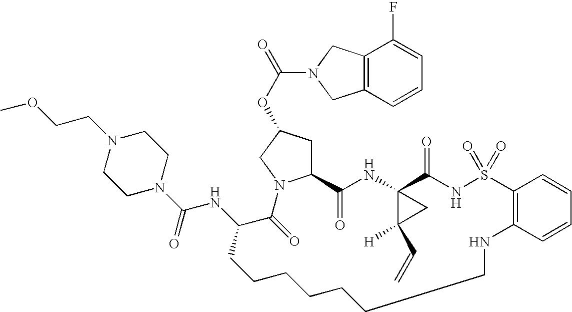 | A-148 |
| 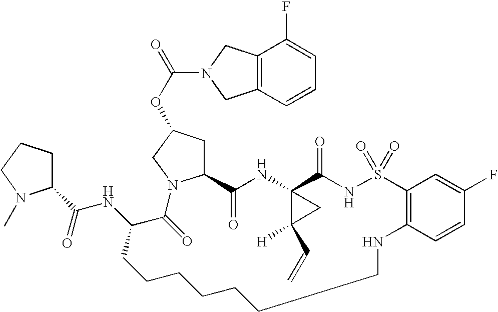 | A-149 |
| 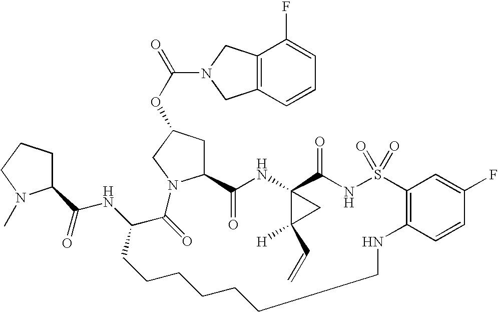 | A-150 |
| 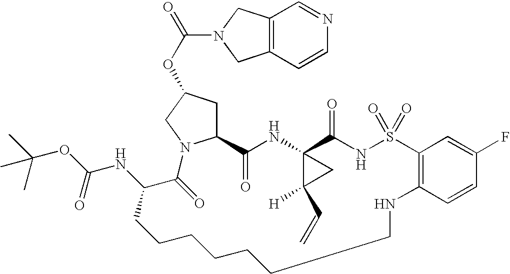 | A-151 |
| 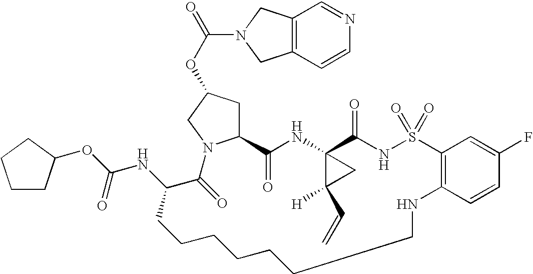 | A-152 |
| 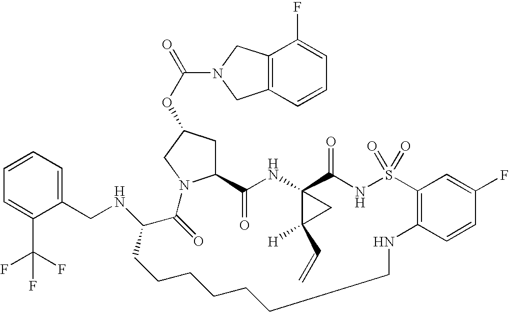 | A-153 |
| 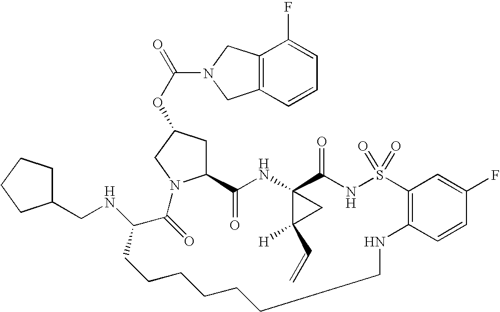 | A-154 |
| 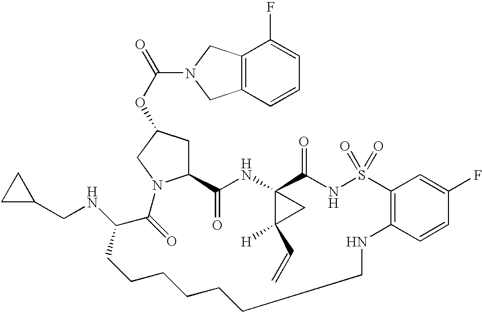 | A-155 |
| 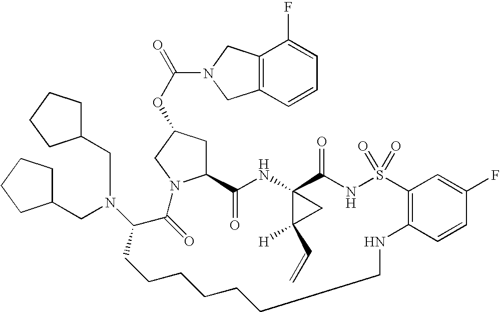 | A-156 |
| 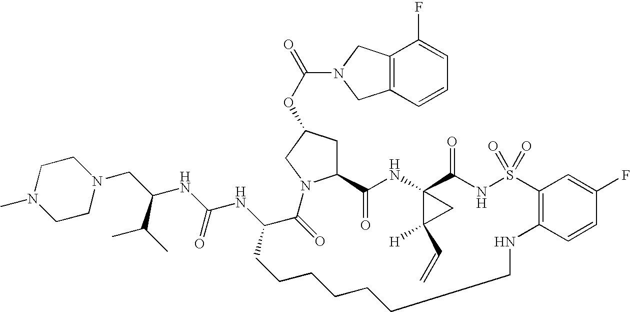 | A-157 |
| 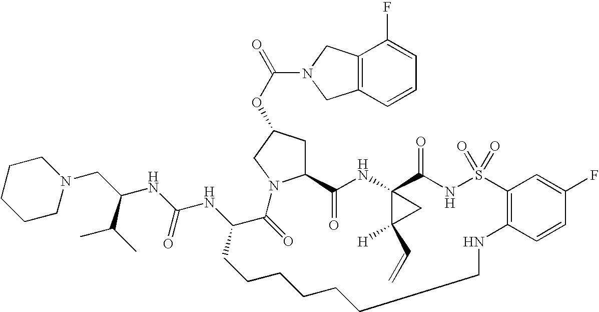 | A-158 |
| 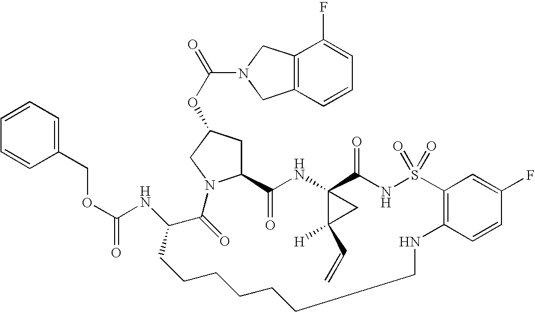 | A-159 |
| 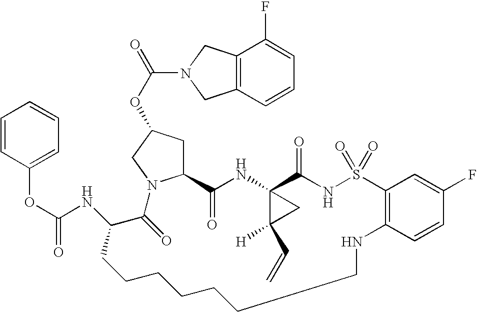 | A-160 |
| 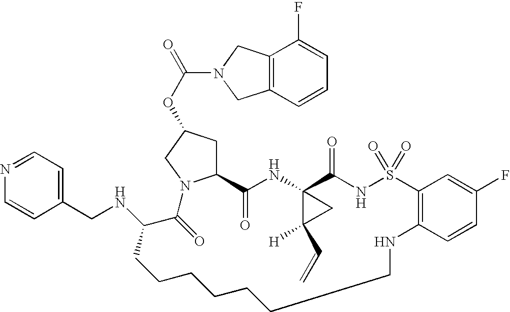 | A-161 |
| 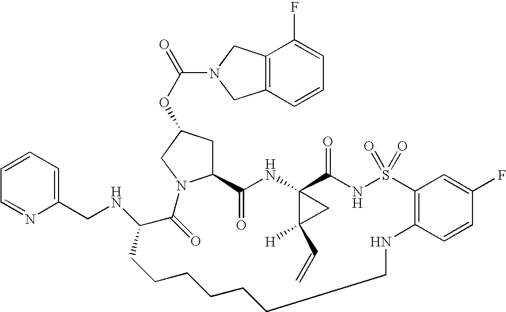 | A-162 |
| 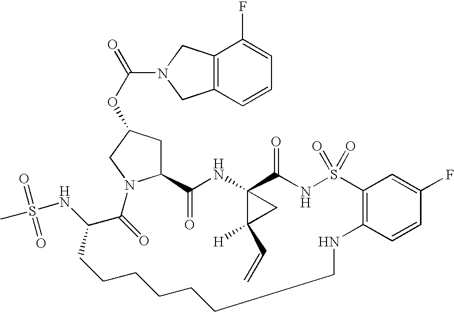 | A-163 |
| 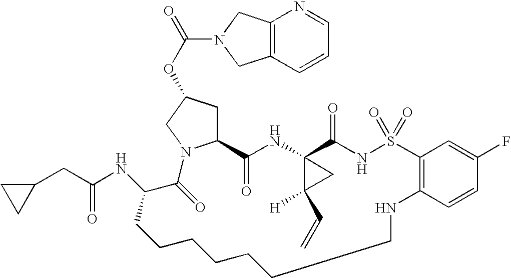 | A-164 |
| 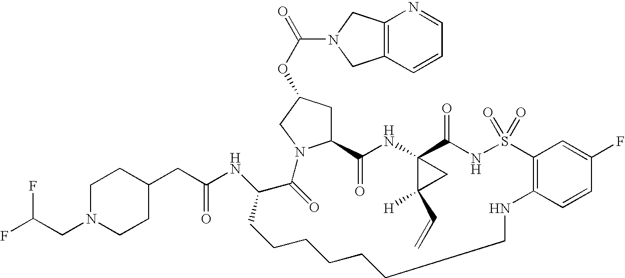 | A-165 |
| 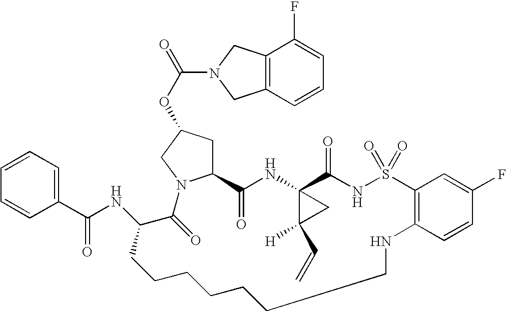 | A-166 |
| 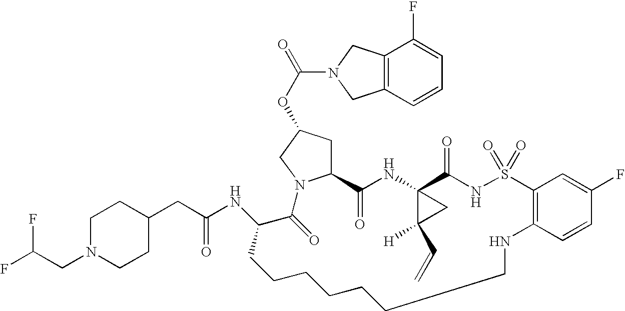 | A-167 |
| 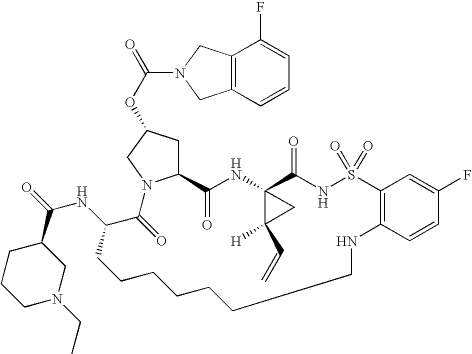 | A-168 |
| 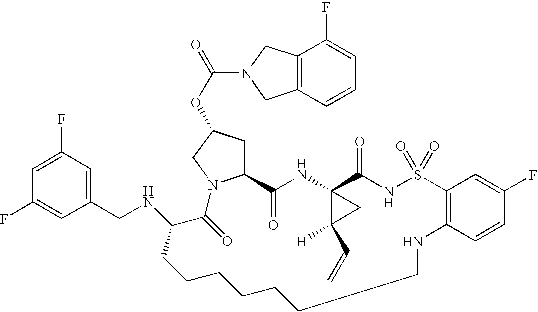 | A-169 |
| 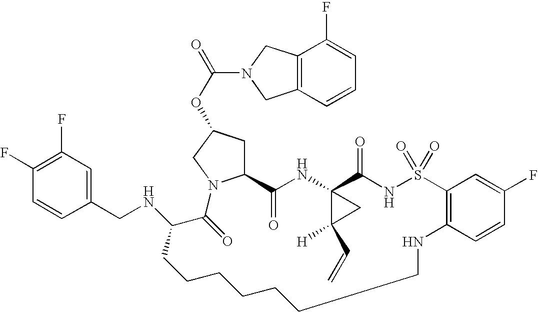 | A-170 |
| 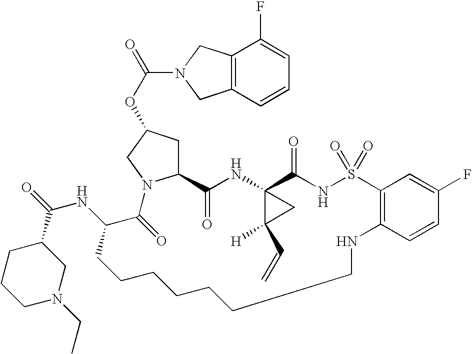 | A-171 |
| 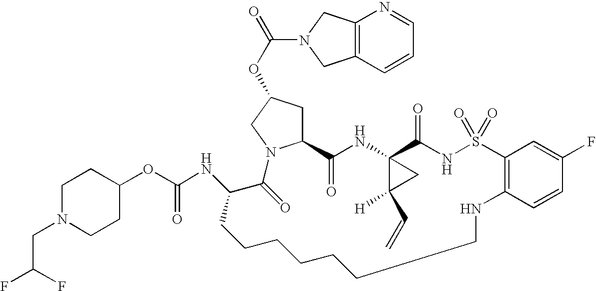 | A-172 |
| 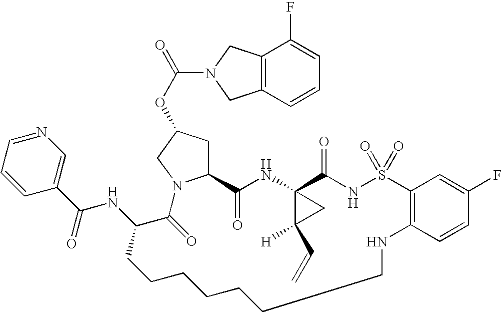 | A-173 |
| 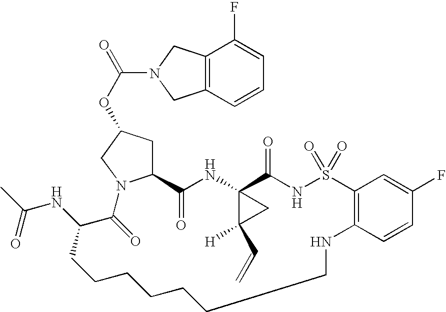 | A-174 |
| 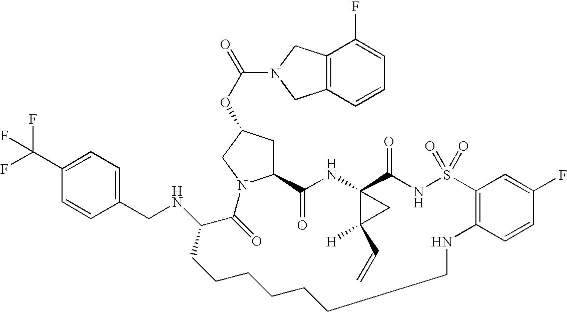 | A-175 |
| 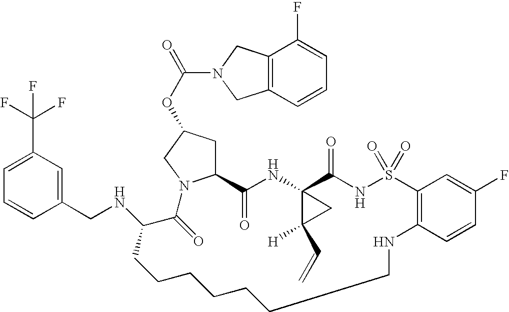 | A-176 |
| 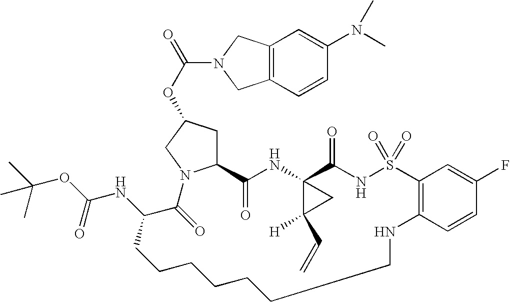 | A-177 |
| 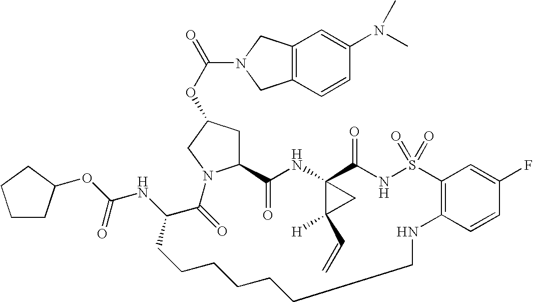 | A-178 |
| 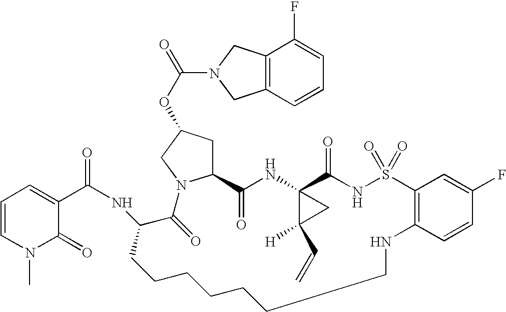 | A-179 |
| 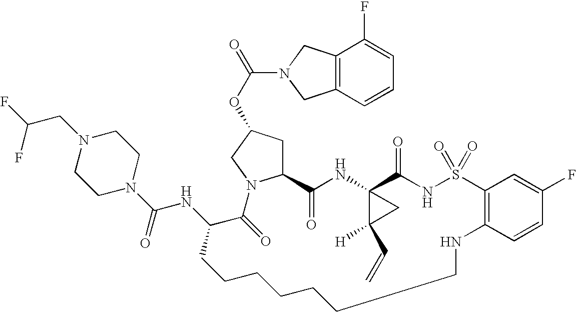 | A-180 |
| 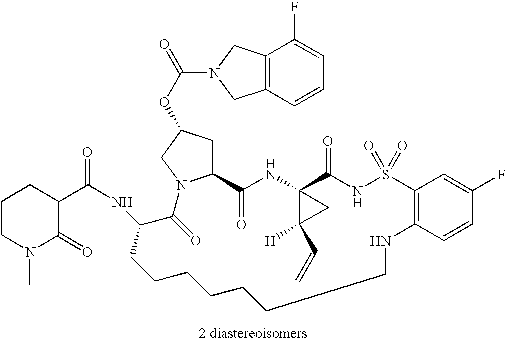 | A-181 |
| 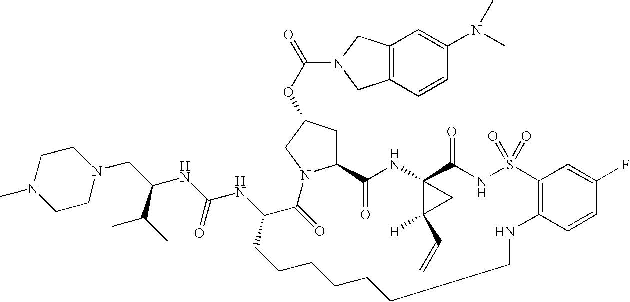 | A-182 |
| 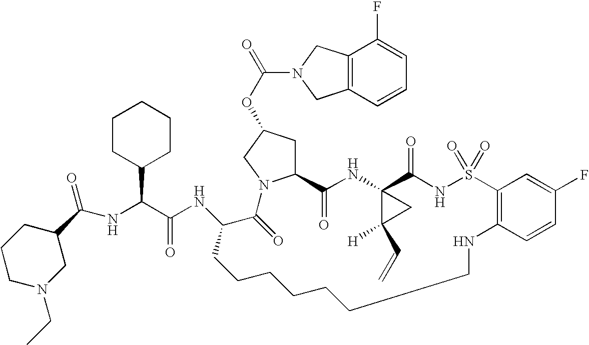 | A-183 |
| 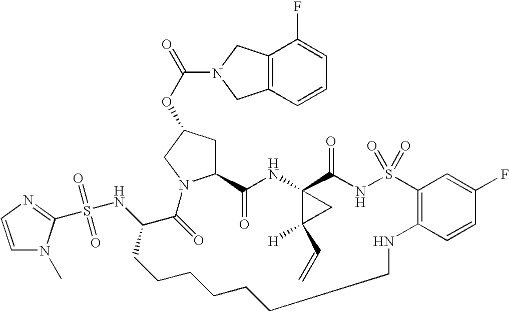 | A-184 |
| 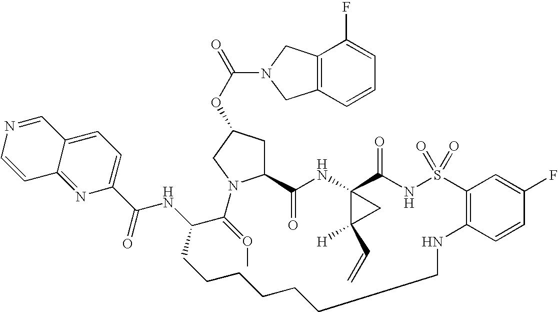 | A-185 |
| 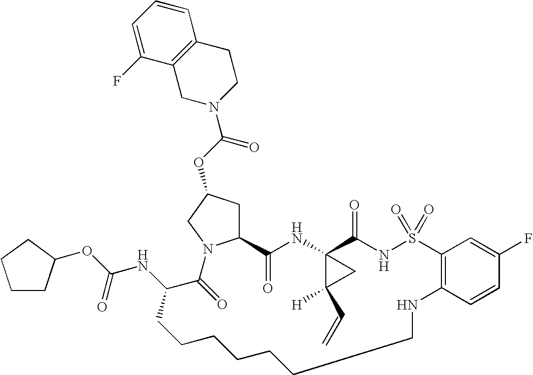 | A-186 |
| 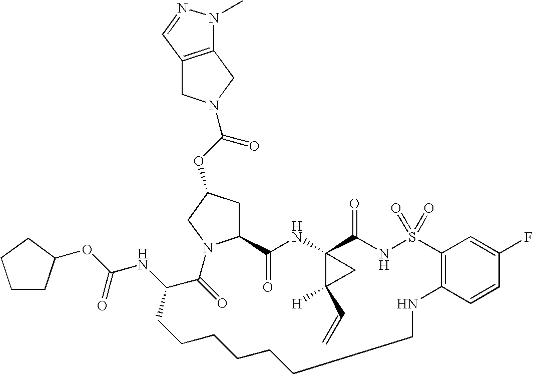 | A-187 |
| 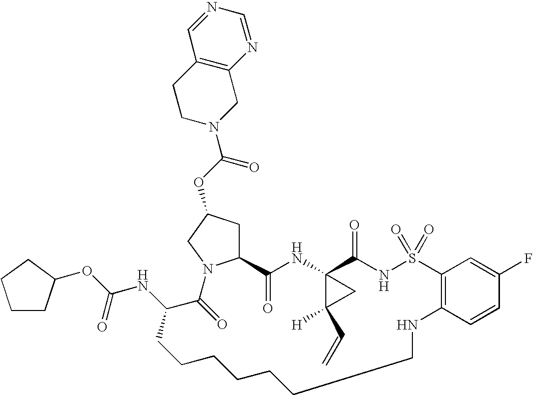 | A-188 |
| 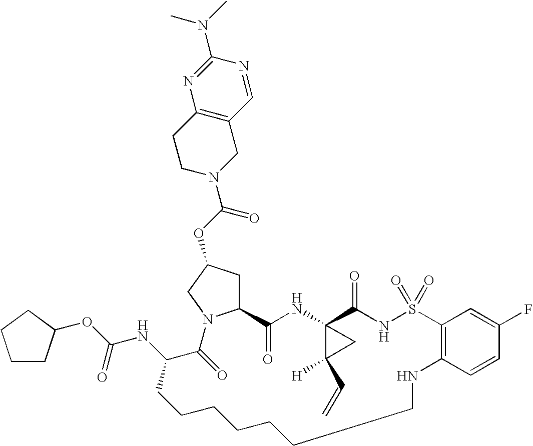 | A-189 |
| 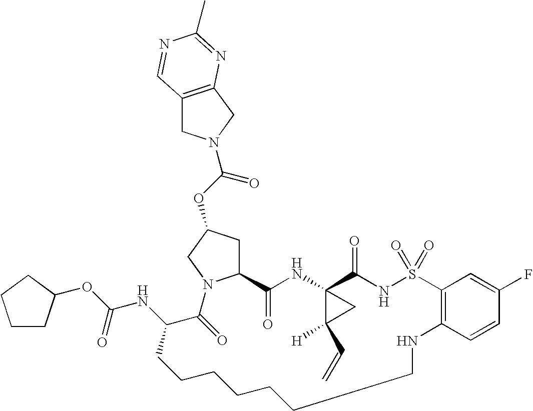 | A-190 |
| 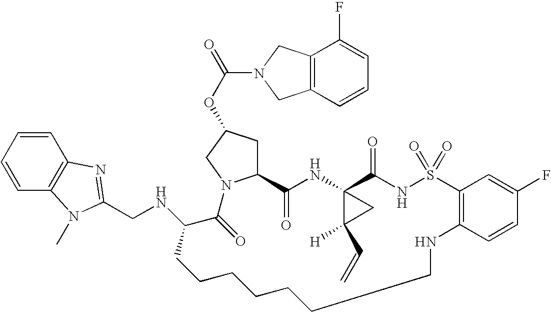 | A-191 |
| 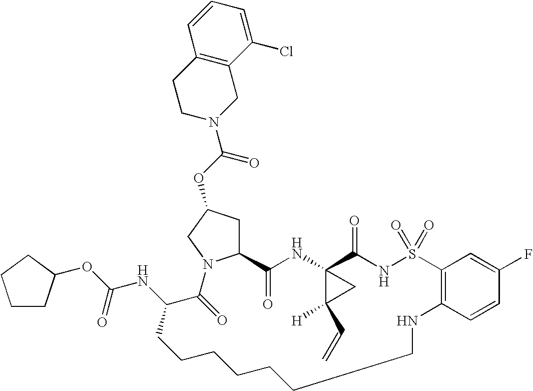 | A-192 |
| 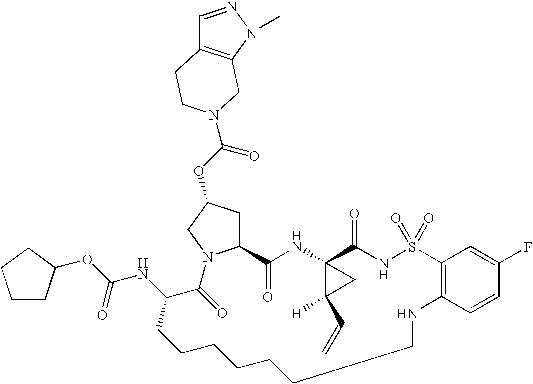 | A-193 |
| 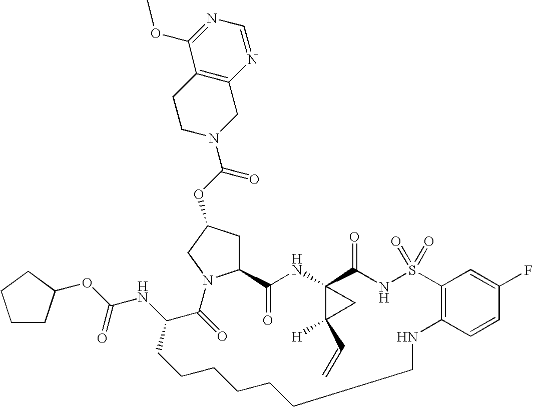 | A-194 |
| 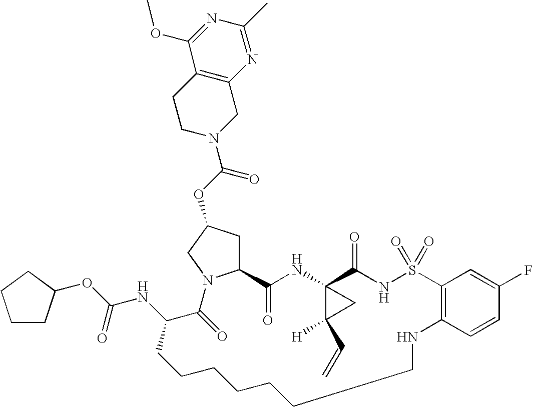 | A-195 |
| 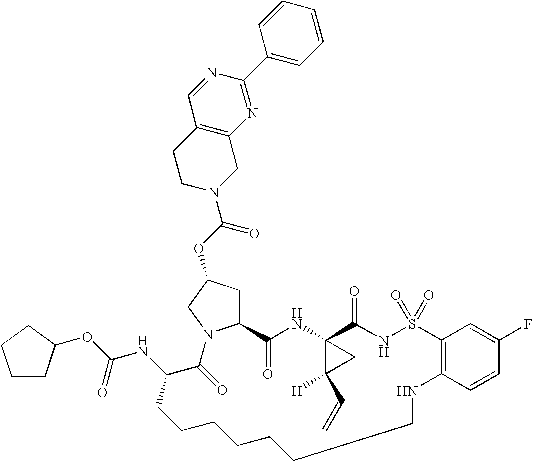 | A-196 |
| 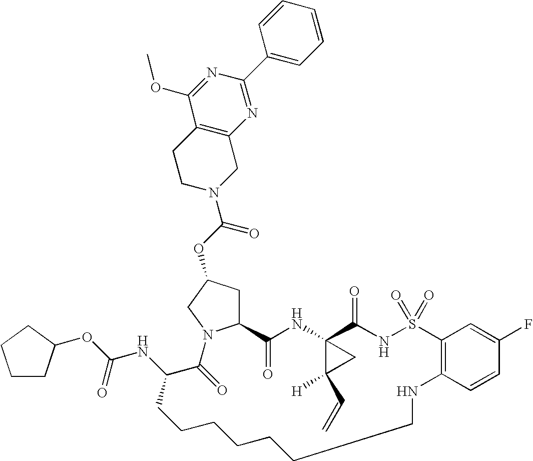 | A-197 |
| 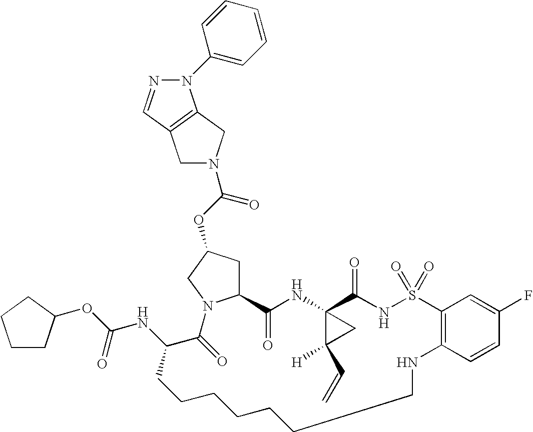 | A-198 |
| 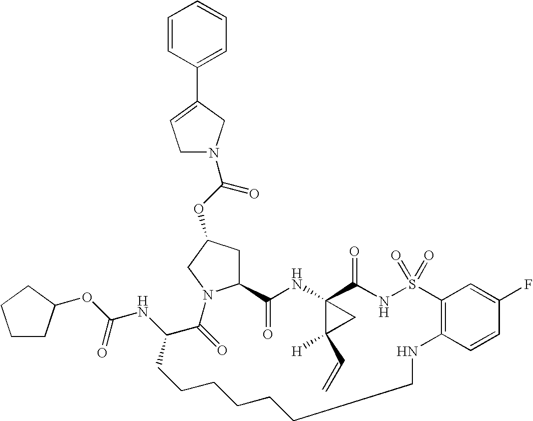 | A-199 |
| 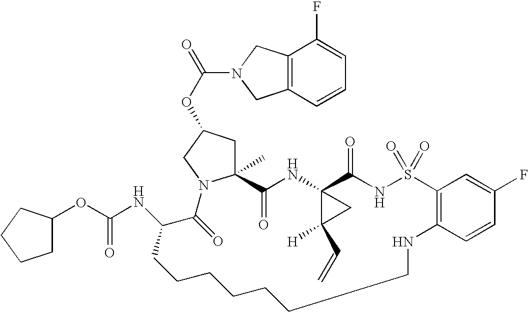 | A-200 |
| 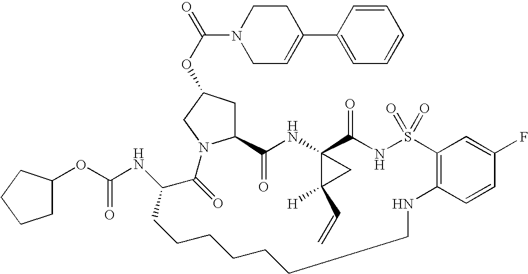 | A-201 |
| 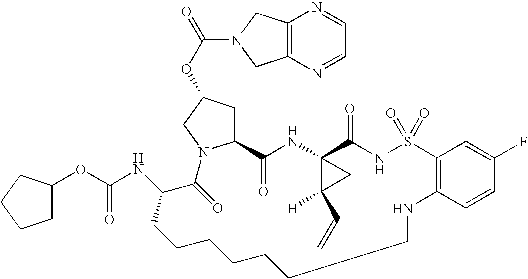 | A-202 |
| 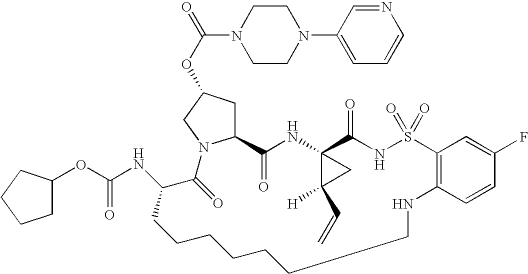 | A-203 |
| 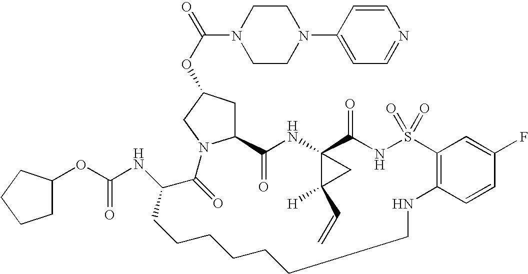 | A-204 |
| 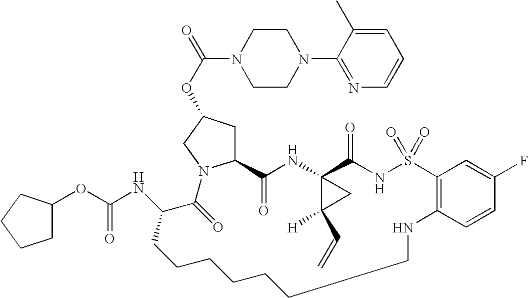 | A-205 |
| 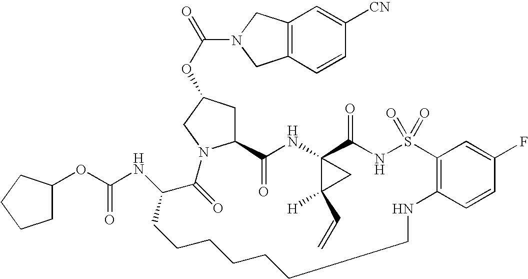 | A-206 |
| 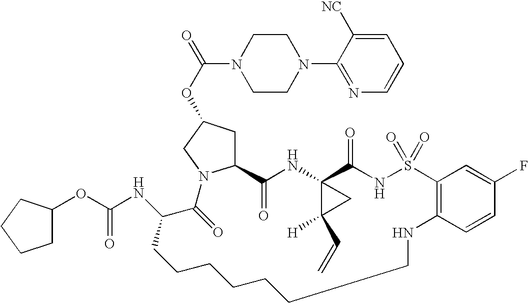 | A-207 |

Certain additional compounds of Formula I (or subformulae thereof) which are contemplated in the present invention include compounds depicted in Table B.

TABLE B

| Structure | Compound No. |
|---|---|
| 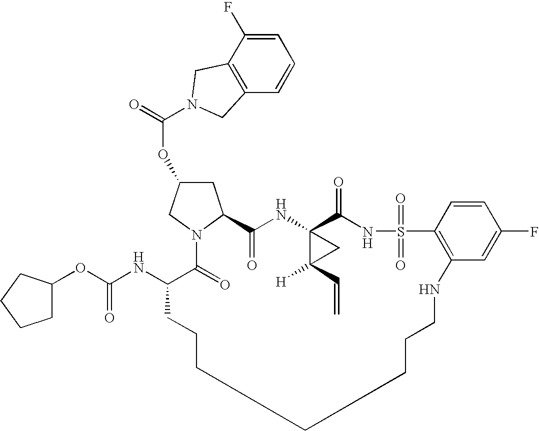 | B-1 |
| 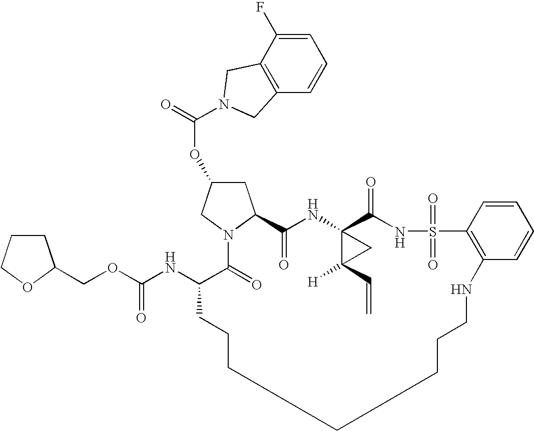 | B-2 |
| 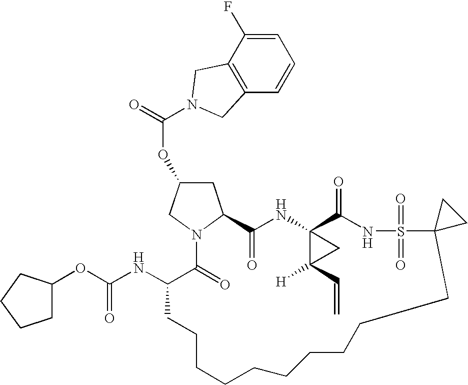 | B-3 |
| 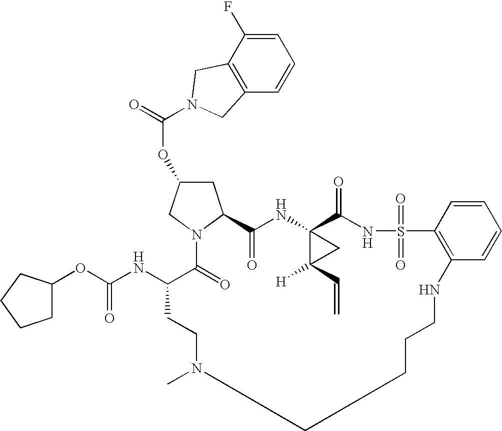 | B-4 |
| 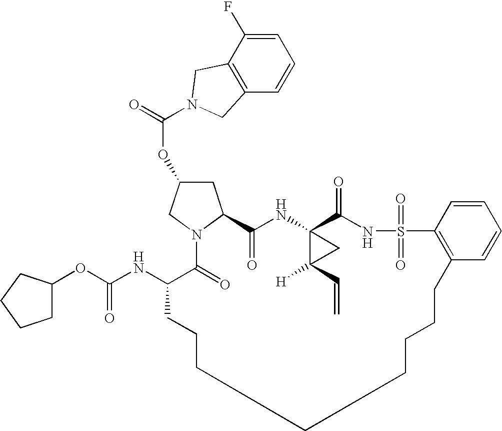 | B-5 |
| 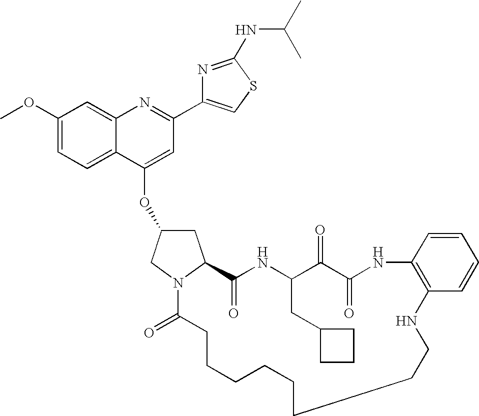 | B-6 |
| 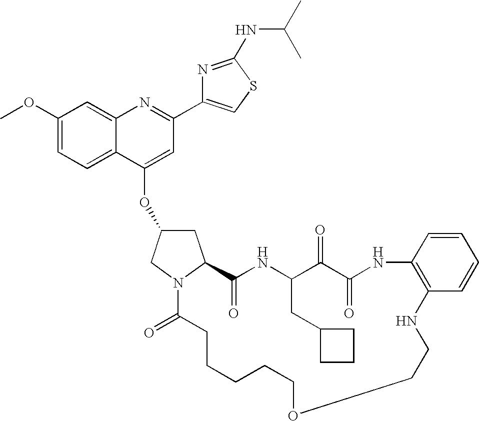 | B-7 |
| 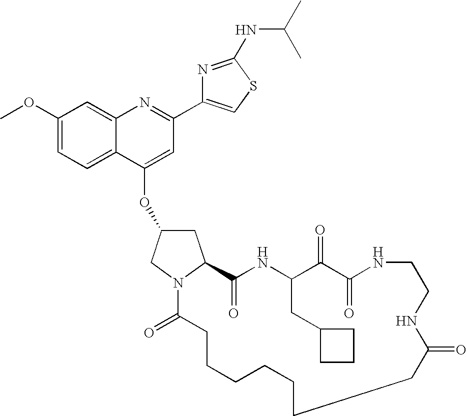 | B-8 |
| 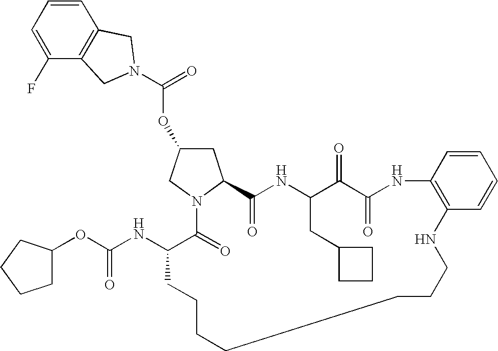 | B-9 |
| 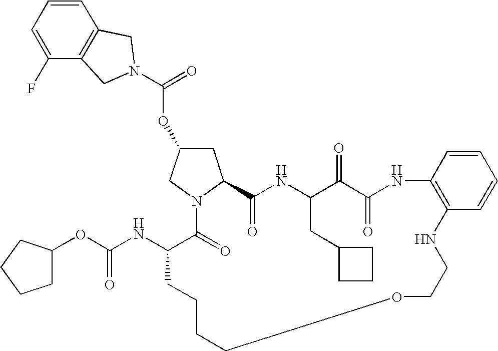 | B-10 |
| 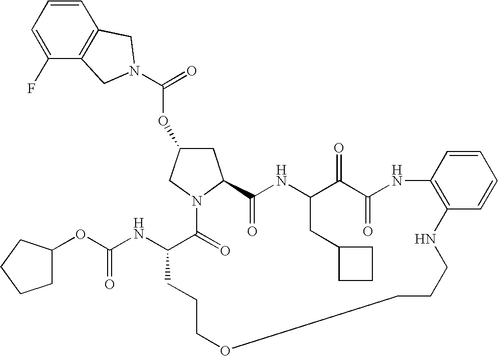 | B-11 |
| 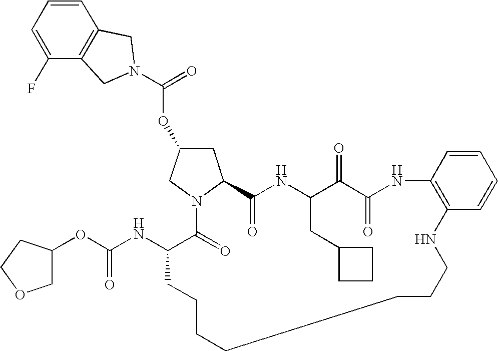 | B-12 |
| 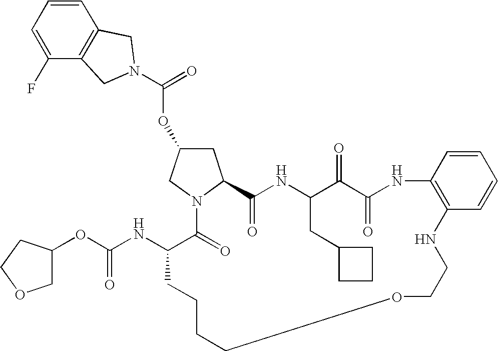 | B-13 |
| 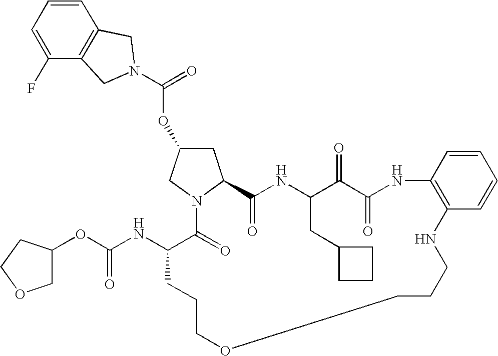 | B-14 |
| 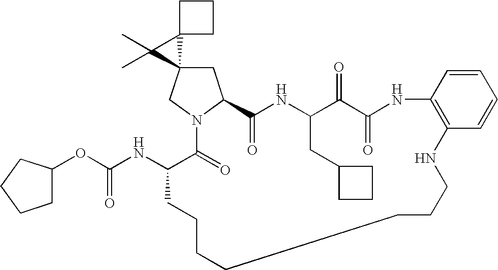 | B-15 |
| 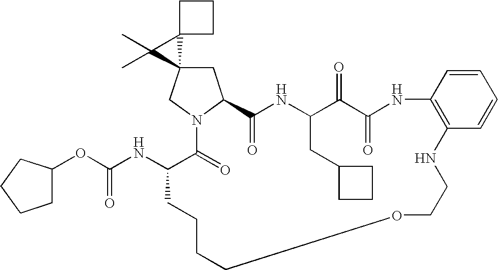 | B-16 |
| 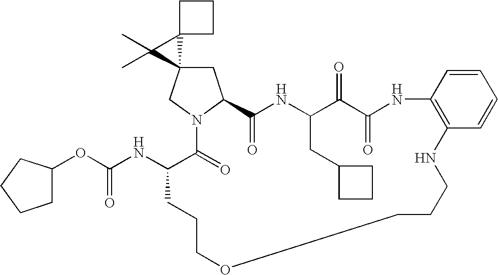 | B-17 |
| 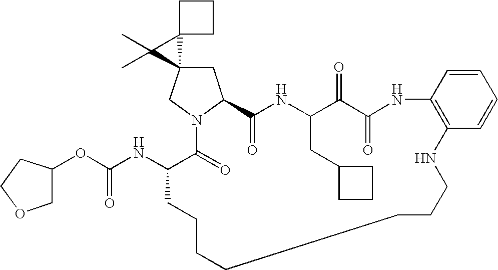 | B-18 |
| 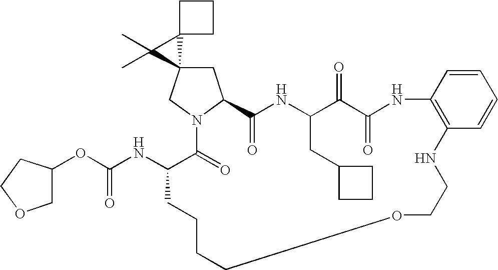 | B-19 |
| 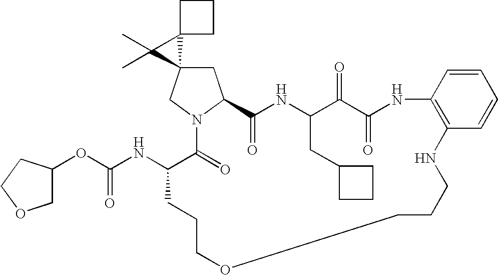 | B-20 |
| 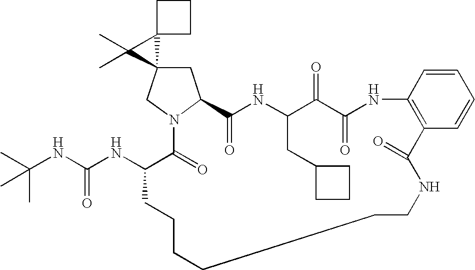 | B-21 |
| 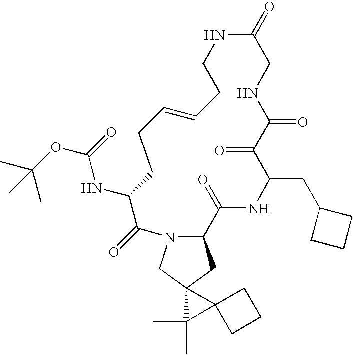 | B-22 |
| 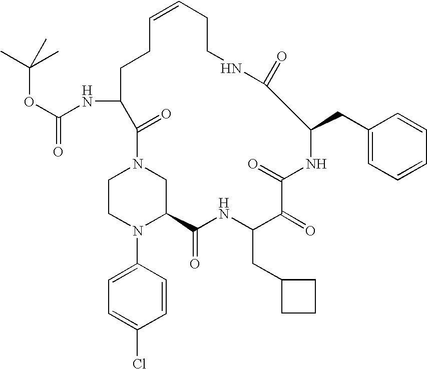 | B-23 |
| 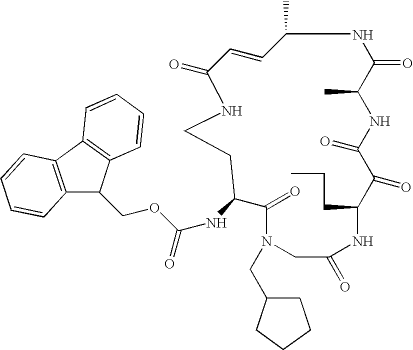 | B-24 |
| 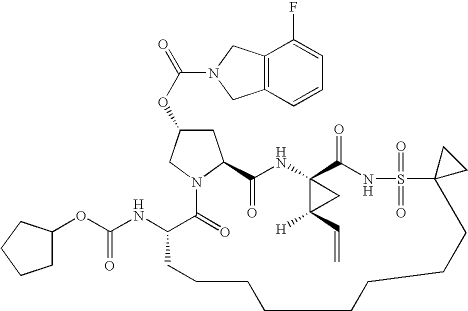 | B-25 |
| 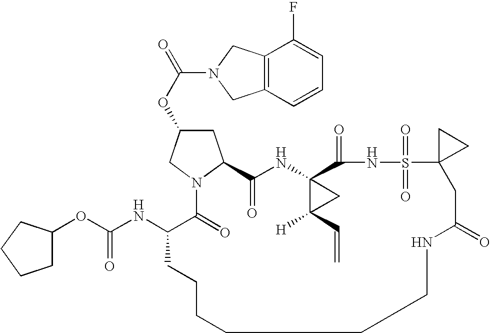 | B-26 |
| 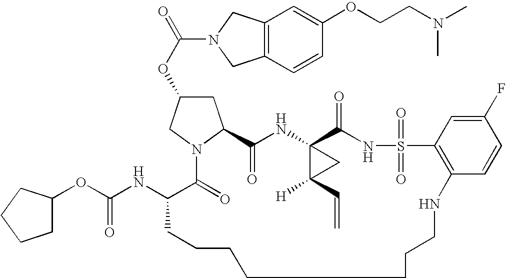 | B-27 |
| 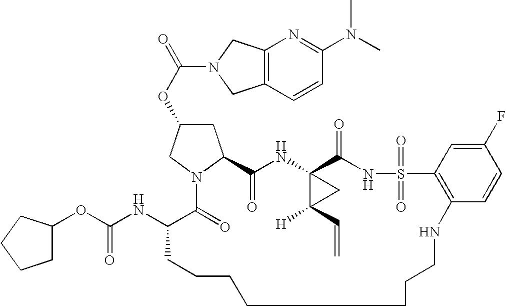 | B-28 |
| 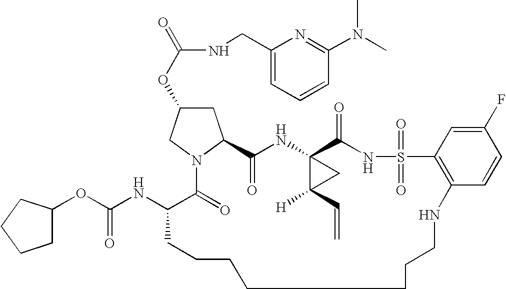 | B-29 |
| 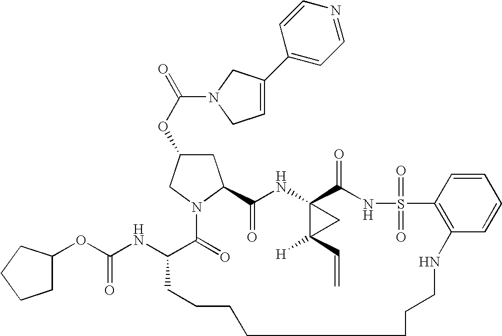 | B-30 |
| 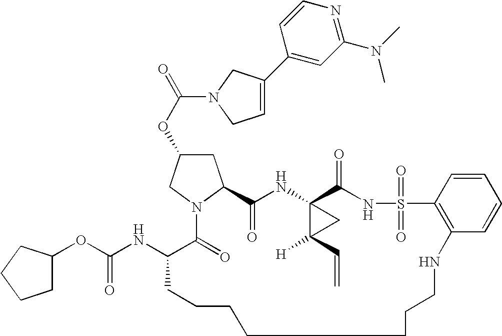 | B-31 |

Certain other compounds of Formula I, and subformulae thereof, include those compounds which contain a fragment selected from the residues of each of Tables C, D, E, F, hand G. Thus, compounds of the invention include all P1-P2 compounds formed by combining all possible permutations of the fragments of Tables C, D, E, F and G wherein the bond ending in an asterisk is the point of attachment P1 and P2 fragments are coupled by condensation of the amino residue on the P1 fragment with the carboxylic acid residue on the P2 fragment. For example, the compound C(1)-D(3)-E(10)-F(4)-G(15) is the compound in which the residue of entry 1 of Table C, the residue of entry 3 of Table D, the residue of entry 10 of Table E, the residue of entry 4 of Table F (where n is 1) and the residue of entry 15 of Table G are combined to form a compound of formula I which has the structure:

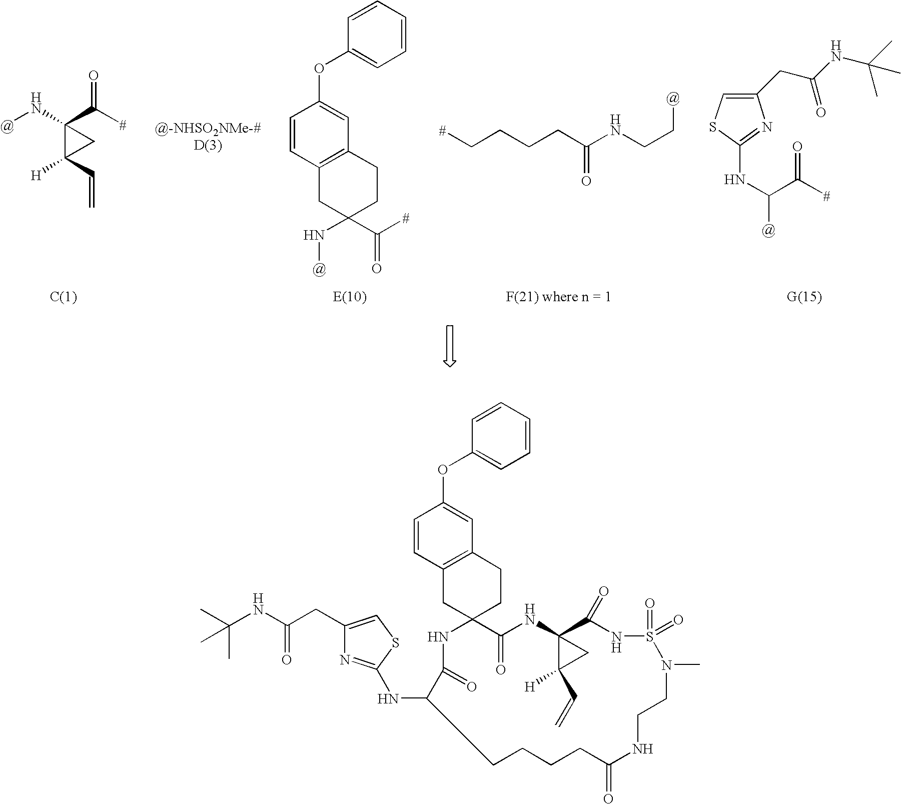

TABLE C
The fragment of Formula 1 has a residue of the formula

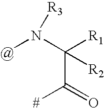

selected from the group consisting of:

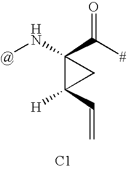

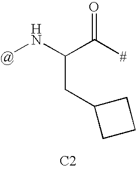

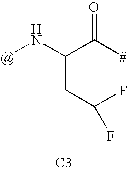

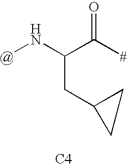

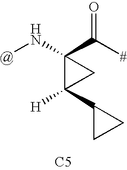

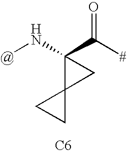

TABLE D
The variable, E, of Formula 1 is a residue
selected from the group consisting of:

| | | |
|---|---|---|
| D1 @-C(O)NH-# | D2 @-NHSO₂-# | D3 @-NHSO₂NMe-# |

TABLE E
The fragment of Formula 1 has a residue of the formula

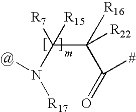

selected from the group consisting of:

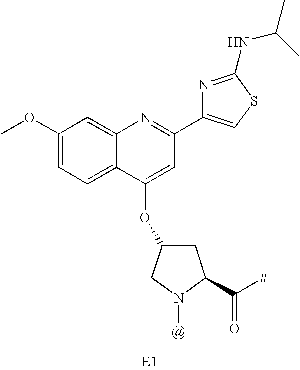

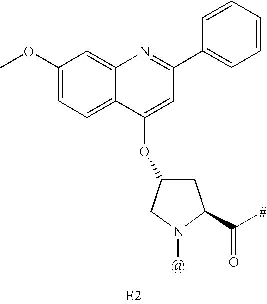

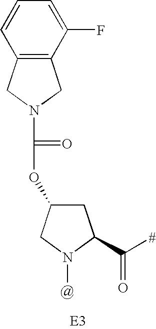

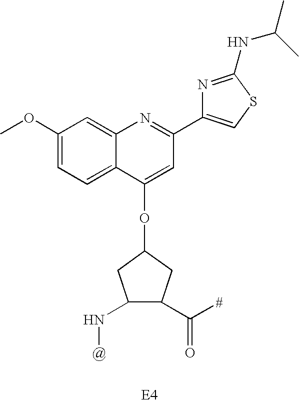

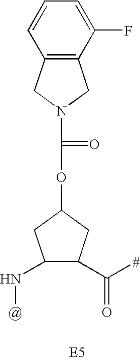

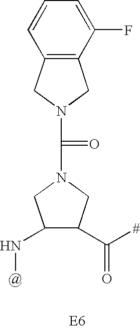

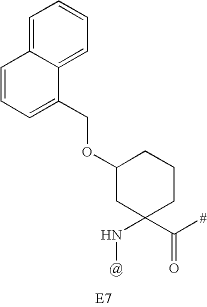

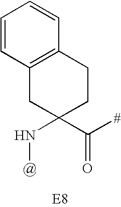

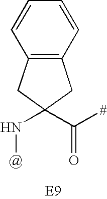

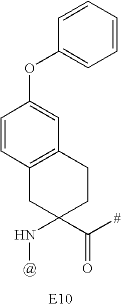

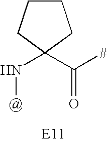

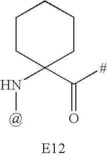

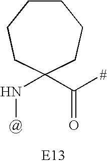

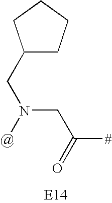

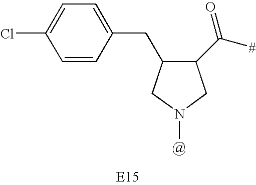

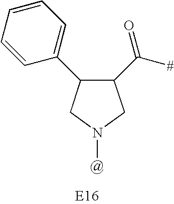

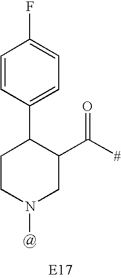

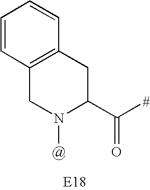

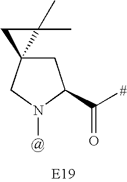

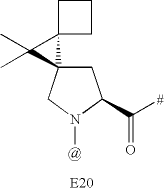

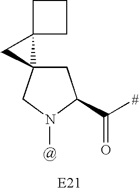

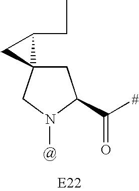

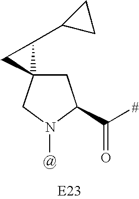

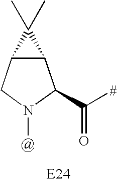

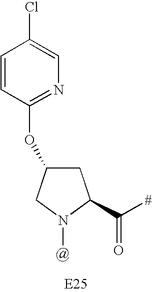

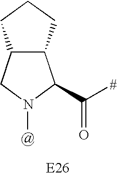

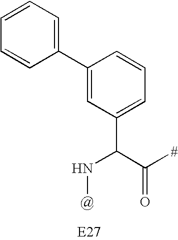

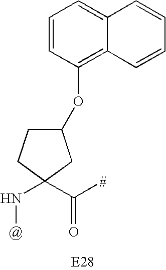

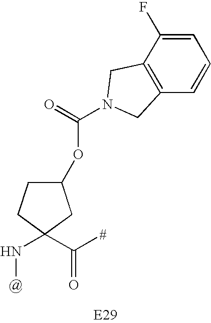

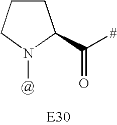

TABLE F
The fragment of Formula 1 has a residue of the formula

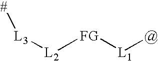

selected from the group consisting of:

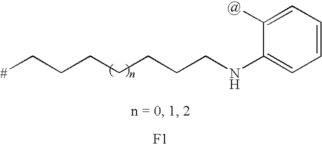

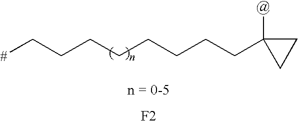

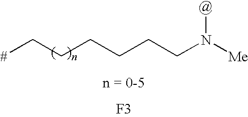

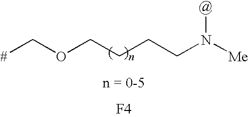

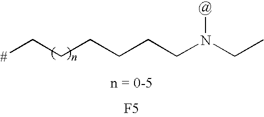

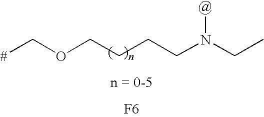

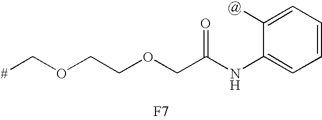

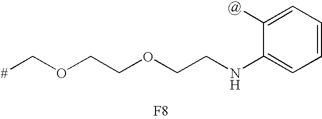

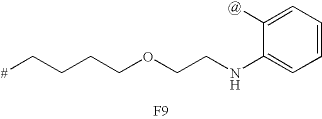

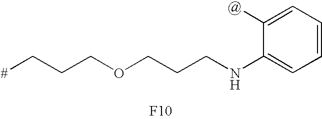

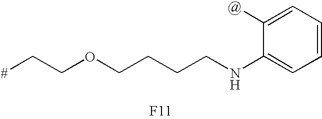

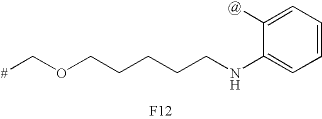

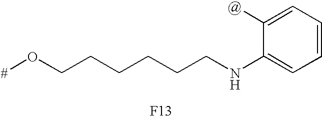

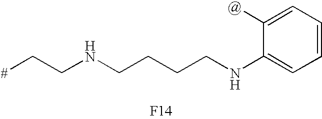

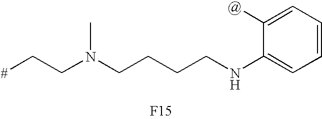

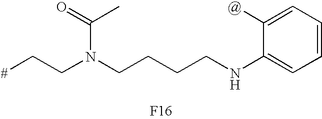

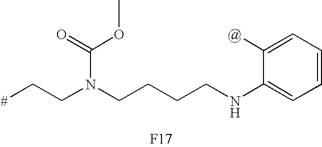

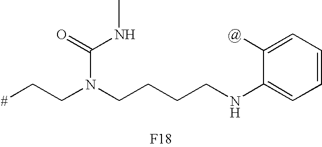

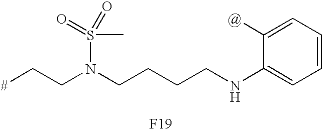

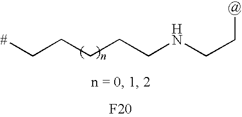

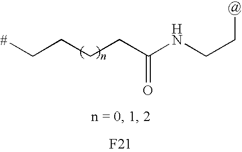

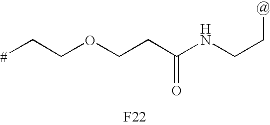

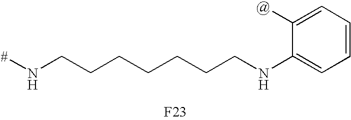

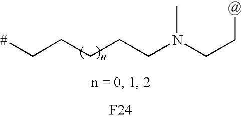

TABLE G
The fragment of Formula I has a residue of the formula

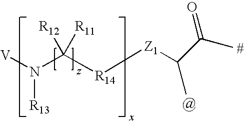

selected from the group consisting of:

| | |
|---|---|
| G1 | 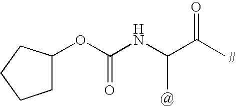 |
| G2 | 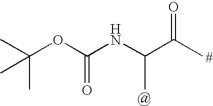 |
| G3 | 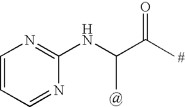 |
| G4 | 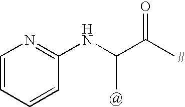 |
| G5 | 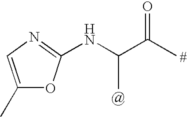 |
| G6 | 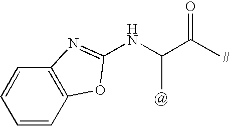 |
| G7 | 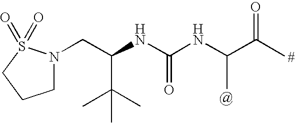 |
| G8 | 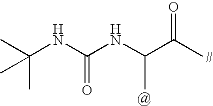 |
| G9 | 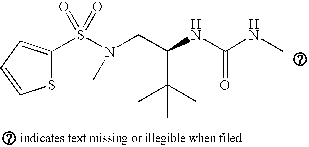 |
| G10 | 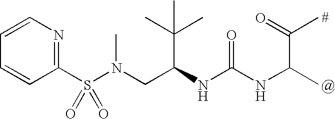 |
| G11 | 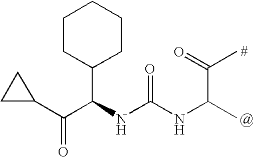 |
| G12 | 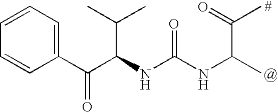 |
| G13 | 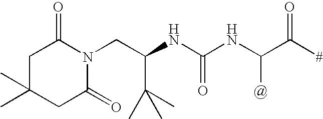 |
| G14 | 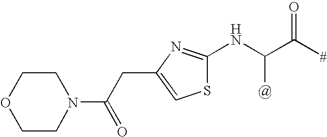 |
| G15 | 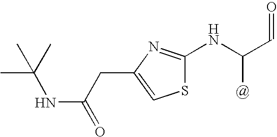 |
| G16 | 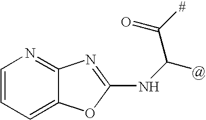 |
| G17 | 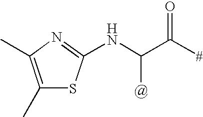 |
| G18 | 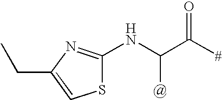 |
| G19 | 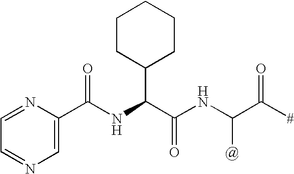 |
| G20 | 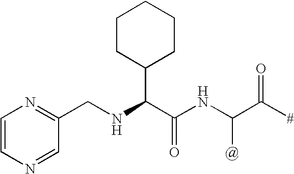 |
| G21 | 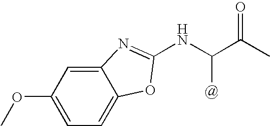 |
| G22 | 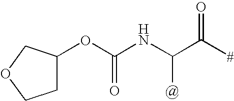 |
| G23 | 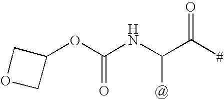 |
| G24 | 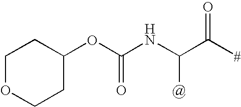 |
| G25 | 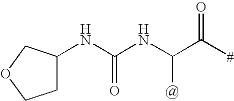 |
| G26 | 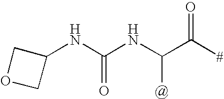 |
| G27 | 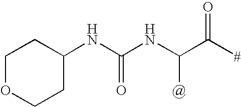 |
| G28 | 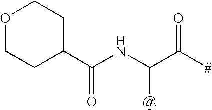 |
| G29 | 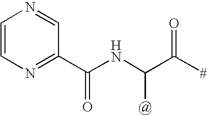 |
| G30 | 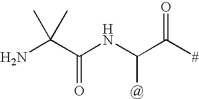 |
| G31 | 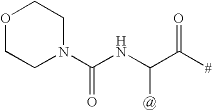 |
| G32 | 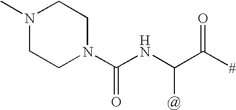 |
| G33 | 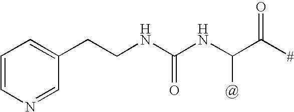 |
| G34 | 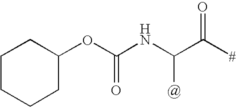 |
| G35 | 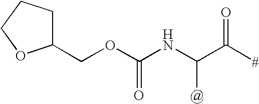 |
| G36 | 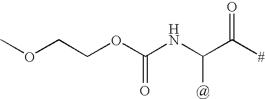 |
| G37 | 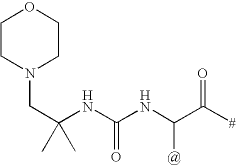 |
| G38 | 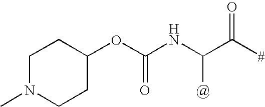 |
| G39 | 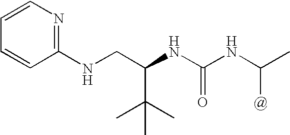 |
| G40 | 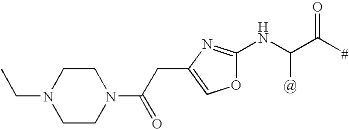 |
| G41 | 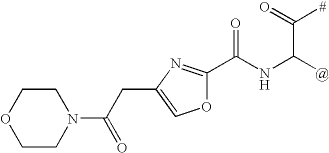 |
| G42 | 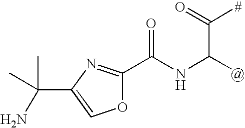 |
| G43 | 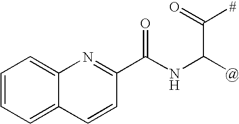 |
| G44 | 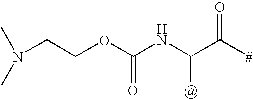 |
| G45 | 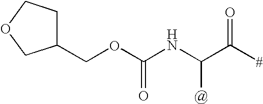 |
| G46 | 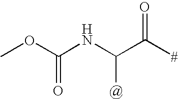 |
| G47 | 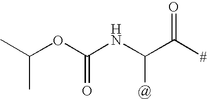 |
| G48 | 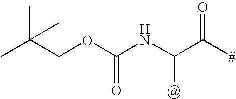 |
| G49 | 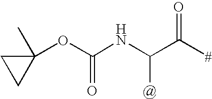 |
| G50 | 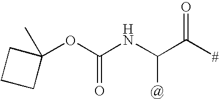 |
| G51 | 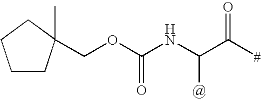 |
| G52 | 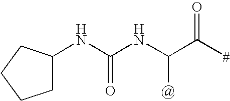 |
| G53 | 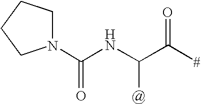 |
| G54 | 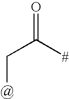 |

Using the HCV NS3-4A protease and Luciferase-HCV replicon assays described in the exemplification section below, certain compounds of the invention (including compounds of Table A depicted above) are found to show IC₅₀ values for HCV inhibition in the range from 10 to more than 100 μM, or 0.5 to 30 μM, or show IC₅₀ values for HCV inhibition of less than 10 μM.

In certain embodiments, a compound of the present invention is further characterized as a modulator of HCV, including a mammalian HCV, and especially including a human HCV. In a preferred embodiment, the compound of the invention is an HCV inhibitor. The terms “HCV-associated state” or “HCV-associated disorder” include disorders and states (e.g., a disease state) that are associated with the activity of HCV, e.g., infection of HCV in a subject. HCV-associated states include HCV-infection, liver cirrhosis, chronic liver disease, hepatocellular carcinoma, cryoglobulinaemia, non-Hodgkin's lymphoma, and a suppressed innate intracellular immune response.

HCV-associated states are often associated with the NS3 serine protease of HCV, which is responsible for several steps in the processing of the HCV polyprotein into smaller functional proteins. NS3 protease forms a heterodimeric complex with the NS4A protein, an essential cofactor that enhances enzymatic activity, and is believed to help anchor HCV to the endoplasmic reticulum. NS3 first autocatalyzes hydrolysis of the NS3-NS4A juncture, and then cleaves the HCV polyprotein intermolecularly at the NS4A-NS4B, NS4B-NS5A and NS5A-NS5B intersections. This process is associated with replication of HCV in a subject. Inhibiting or modulating the activity of one or more of the NS3, NS4A, NS4B, NS5A and NS5B proteins will inhibit or modulate replication of HCV in a subject, thereby preventing or treating the HCV-associated state. In a particular embodiment, the HCV-associated state is associated with the activity of the NS3 protease. In another particular embodiment, the HCV-associated state is associated with the activity of NS3-NS4A heterodimeric complex.

In one embodiment, the compounds of the invention are NS3/NS4A protease inhibitors. In another embodiment, the compounds of the invention are NS2/NS3 protease inhibitors.

Without being bound by theory, it is believed that the disruption of the above protein-protein interactions by the compounds of the invention will interfere with viral polyprotein processing by the NS3 protease and thus viral replication.

HCV-associated disorders also include HCV-dependent diseases. HVC-dependent diseases include, e.g., any disease or disorder that depend on or related to activity or misregulation of at least one strain of HCV.

The present invention includes treatment of HCV-associated disorders as described above, but the invention is not intended to be limited to the manner by which the compound performs its intended function of treatment of a disease. The present invention includes treatment of diseases described herein in any manner that allows treatment to occur, e.g., HCV infection.

In a related embodiment, the compounds of the invention can be useful for treating diseases related to HIV, as well as HIV infection and AIDS (Acquired Immune Deficiency Syndrome).

In certain embodiments, the invention provides a pharmaceutical composition of any of the compounds of the present invention. In a related embodiment, the invention provides a pharmaceutical composition of any of the compounds of the present invention and a pharmaceutically acceptable carrier or excipient of any of these compounds. In certain embodiments, the invention includes the compounds as novel chemical entities.

In one embodiment, the invention includes a packaged HCV-associated disorder treatment. The packaged treatment includes a compound of the invention packaged with instructions for using an effective amount of the compound of the invention for an intended use.

The compounds of the present invention are suitable as active agents in pharmaceutical compositions that are efficacious particularly for treating HCV-associated disorders. The pharmaceutical composition in various embodiments has a pharmaceutically effective amount of the present active agent along with other pharmaceutically acceptable excipients, carriers, fillers, diluents and the like. The phrase, “pharmaceutically effective amount” as used herein indicates an amount necessary to administer to a host, or to a cell, issue, or organ of a host, to achieve a therapeutic result, especially an anti-HCV effect, e.g., inhibition of proliferation of the HCV virus, or of any other HCV-associated disease.

In one embodiment, the diseases to be treated by compounds of the invention include, for example, HCV infection, liver cirrhosis, chronic liver disease, hepatocellular carcinoma, cryoglobulinaemia, non-Hodgkin's lymphoma, and a suppressed innate intracellular immune response.

In other embodiments, the present invention provides a method for inhibiting the activity of HCV. The method includes contacting a cell with any of the compounds of the present invention. In a related embodiment, the method further provides that the compound is present in an amount effective to selectively inhibit the activity of one or more of the NS3, NS4A, NS4B, NS5A and NS5B proteins. In another related embodiment, the method provides that the compound is present in an amount effective to diminish the HCV RNA load in a subject.

In other embodiments, the present invention provides a use of any of the compounds of the invention for manufacture of a medicament to treat HCV infection in a subject.

In other embodiments, the invention provides a method of manufacture of a medicament, including formulating any of the compounds of the present invention for treatment of a subject.

### DEFINITIONS

The term “treat,” “treated,” “treating” or “treatment” includes the diminishment or alleviation of at least one symptom associated or caused by the state, disorder or disease being treated. In certain embodiments, the treatment comprises the induction of an HCV-inhibited state, followed by the activation of the HCV-modulating compound, which would in turn diminish or alleviate at least one symptom associated or caused by the HCV-associated state, disorder or disease being treated. For example, treatment can be diminishment of one or several symptoms of a disorder or complete eradication of a disorder.

The term “subject” is intended to include organisms, e.g., prokaryotes and eukaryotes, which are capable of suffering from or afflicted with an HCV-associated disorder. Examples of subjects include mammals, e.g., humans, dogs, cows, horses, pigs, sheep, goats, cats, mice, rabbits, rats, and transgenic non-human animals. In certain embodiments, the subject is a human, e.g., a human suffering from, at risk of suffering from, or potentially capable of suffering from an HCV-associated disorder, and for diseases or conditions described herein, e.g., HCV infection. In another embodiment, the subject is a cell.

The language “HCV-modulating compound,” “modulator of HCV” or “HCV inhibitor” refers to compounds that modulate, e.g., inhibit, or otherwise alter, the activity of HCV. Similarly, an “NS3/NS4A protease inhibitor,” or an “NS2/NS3 protease inhibitor” refers to a compound that modulates, e.g., inhibits, or otherwise alters, the interaction of these proteases with one another. Examples of HCV-modulating compounds include compounds of Formula I, as well as Table A and Table B (including pharmaceutically acceptable salts thereof, as well as enantiomers, stereoisomers, rotamers, tautomers, diastereomers, or racemates thereof).

Additionally, the method includes administering to a subject an effective amount of an HCV-modulating compound of the invention, e.g., HCV-modulating compounds of Formula I, as well as Table A and Table B (including pharmaceutically acceptable salts thereof, as well as enantiomers, stereoisomers, rotamers, tautomers, diastereomers, or racemates thereof).

The term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl(alicyclic) groups (cyclopropyl, cyclopentyl, cyclohexyl, cycloheptyl, cyclooctyl), alkyl substituted cycloalkyl groups, and cycloalkyl substituted alkyl groups. The term “alkyl” also includes alkenyl groups and alkynyl groups. Furthermore, the expression “Cₓ-C_{y}-alkyl”, wherein x is 1-5 and y is 2-10 indicates a particular alkyl group (straight- or branched-chain) of a particular range of carbons. For example, the expression C₁-C₄-alkyl includes, but is not limited to, methyl, ethyl, propyl, butyl, isopropyl, tert-butyl, isobutyl and sec-butyl. Moreover, the term C₃₋₆-cycloalkyl includes, but is not limited to, cyclopropyl, cyclopentyl, and cyclohexyl. As discussed below, these alkyl groups, as well as cycloalkyl groups, may be further substituted. “C₀-Cₙalkyl” refers to a single covalent bond (C₀) or an alkyl group having from 1 to n carbon atoms; for example “C₀-C₄alkyl” refers to a single covalent bond or a C₁-C₄alkyl group; “C₀-C₈alkyl” refers to a single covalent bond or a C₁-C₈alkyl group. In some instances, a substituent of an alkyl group is specifically indicated. For example, “C₁-C₄hydroxyalkyl” refers to a C₁-C₄alkyl group that has at least one hydroxy substituent.

“Alkylene” refers to a divalent alkyl group, as defined above. C₀-C₄alkylene is a single covalent bond or an alkylene group having from 1 to 4 carbon atoms; and C₀-C₆alkylene is a single covalent bond or an alkylene group having from 1 to 6 carbon atoms. “Alkenylene” and “Alkynylene” refer to divalent alkenyl and alkynyl groups respectively, as defined above.

The term alkyl further includes alkyl groups which can further include oxygen, nitrogen, sulfur or phosphorous atoms replacing one or more carbons of the hydrocarbon backbone. In an embodiment, a straight chain or branched chain alkyl has 10 or fewer carbon atoms in its backbone (e.g., C₁-C₁₀ for straight chain, C₃-C₁₀ for branched chain), and more preferably 6 or fewer carbons.

A “cycloalkyl” is a group that comprises one or more saturated and/or partially saturated rings in which all ring members are carbon, such as cyclopropyl, cyclobutyl, cyclopentyl, cyclohexyl, cycloheptyl, cyclooctyl, adamantyl, decahydro-naphthalenyl, octahydro-indenyl, and partially saturated variants of the foregoing, such as cyclohexenyl. Cycloalkyl groups do not comprise an aromatic ring or a heterocyclic ring. Certain cycloalkyl groups are C₃-C₈cycloalkyl, in which the group contains a single ring with from 3 to 8 ring members. A “(C₃-C₈cycloalkyl)C₀-C₄alkyl” is a C₃-C₈cycloalkyl group linked via a single covalent bond or a C₁-C₄alkylene group. In certain aspects, C₃₋₆-cycloalkyl groups are substituted one or more times (or preferably between one and five times) with substitutents independently selected from a halogen atom, aryl, heteroaryl, trihalomethyl, C₁₋₄-alkoxy or C₁₋₄-alkyl.

Moreover, alkyl (e.g., methyl, ethyl, propyl, butyl, pentyl, hexyl, etc.) include both “unsubstituted alkyl” and “substituted alkyl”, the latter of which refers to alkyl moieties having substituents replacing a hydrogen on one or more carbons of the hydrocarbon backbone, which allow the molecule to perform its intended function.

The term “substituted” is intended to describe moieties having substituents replacing a hydrogen on one or more atoms, e.g. C, O or N, of a molecule. Such substituents can include, for example, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkyl amino; dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, stilfonato, sulfamoyl, sulfonamido, nitro, trifluoromethyl, cyano, azido, heterocyclyl, alkylaryl, morpholino, phenol, benzyl, phenyl, piperizine, cyclopentane, cyclohexane, pyridine, 5H-tetrazole, triazole, piperidine, or an aromatic or heteroaromatic moiety.

Further examples of substituents of the invention, which are not intended to be limiting, include moieties selected from straight or branched alkyl (preferably C₁-C₅), cycloalkyl (preferably C₃-C₈), alkoxy (preferably C₁-C₆), thioalkyl (preferably C₁-C₆), alkenyl (preferably C₂-C₆), alkynyl (preferably C₂-C₆), heterocyclic, carbocyclic, aryl (e.g., phenyl), aryloxy (e.g., phenoxy), aralkyl (e.g., benzyl), aryloxyalkyl (e.g., phenyloxyalkyl), arylacetamidoyl, alkylaryl, heteroaralkyl, alkylcarbonyl and arylcarbonyl or other such acyl group, heteroarylcarbonyl, or heteroaryl group, (CR′R″)₀₋₃NR′R″ (e.g., —NH₂), (CR′R″)₀₋₃CN (e.g., —CN), —NO₂, halogen (e.g., —F, —Cl, —Br, or —I), (CR′R″)₀₋₃C(halogen)₃ (e.g., —CF₃), (CR′R″)₀₋₃CH(halogen)₂, (CR′R″)₀₋₃CH₂(halogen), (CR′R″)₀₋₃CONR′R″, (CR′R″)₀₋₃(CNH)NR′R″, (CR′R″)₀₋₃S(O)₁₋₂NR′R″, (CR′R″)₀₋₃CHO, (CR′R″)₀₋₃O(CR′R″)₀₋₃H, (CR′R″)₀₋₃S(O)₀₋₃R′ (e.g., —SO₃H, —OSO₃H), (CR′R″)₀₋₃O(CR′R″)₀₋₃H (e.g., —CH₂OCH₃ and —OCH₃), (CR′R″)₀₋₃S(CR′R″)₀₋₃H (e.g., —SH and —SCH₃), (CR′R″)₀₋₃OH (e.g., —OH), (CR′R″)₀₋₃COR′, (CR′R″)₀₋₃(substituted or unsubstituted phenyl), (CR′R″)₀₋₃(C₃-C₈ cycloalkyl), (CR′R″)₀₋₃CO₂R′ (e.g., —CO₂H), or (CR′R″)₀₋₃OR′ group, or the side chain of any naturally occurring amino acid; wherein R′ and R″ are each independently hydrogen, a C₁-C₅ alkyl, C₂-C₅ alkenyl, C₂-C₅ alkynyl, or aryl group. Such substituents can include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, oxime, sulfhydryl, alkylthio, arylthio, thiocarboxylate, sulfates, sulfonato, sulfamoyl, sulfonamido, nitro, trifluoromethyl, cyano, azido, heterocyclyl, or an aromatic or heteroaromatic moiety. In certain embodiments, a carbonyl moiety (C═O) may be further derivatized with an oxime moiety, e.g., an aldehyde moiety may be derivatized as its oxime (—C═N—OH) analog. It will be understood by those skilled in the art that the moieties substituted on the hydrocarbon chain can themselves be substituted, if appropriate. Cycloalkyls can be further substituted, e.g., with the substituents described above. An “aralkyl” moiety is an alkyl substituted with an aryl (e.g., phenylmethyl (i.e., benzyl)).

The term “alkenyl” includes unsaturated aliphatic groups analogous in length and possible substitution to the alkyls described above, but which contain at least one double bond.

For example, the term “alkenyl” includes straight-chain alkenyl groups (e.g., ethenyl, propenyl, butenyl, pentenyl, hexenyl, heptenyl, octenyl, nonenyl, decenyl, etc.), branched-chain alkenyl groups, cycloalkenyl(alicyclic) groups (cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. The term alkenyl further includes alkenyl groups that include oxygen, nitrogen, sulfur or phosphorous atoms replacing one or more carbons of the hydrocarbon backbone. In certain embodiments, a straight chain or branched chain alkenyl group has 6 or fewer carbon atoms in its backbone (e.g., C₂-C₆ for straight chain, C₃-C₆ for branched chain). Likewise, cycloalkenyl groups may have from 3-8 carbon atoms in their ring structure, and more preferably have 5 or 6 carbons in the ring structure. The term C₂-C₆ includes alkenyl groups containing 2 to 6 carbon atoms.

Moreover, the term alkenyl includes both “unsubstituted alkenyls” and “substituted alkenyls”, the latter of which refers to alkenyl moieties having substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “alkynyl” includes unsaturated aliphatic groups analogous in length and possible substitution to the alkyls described above, but which contain at least one triple bond.

For example, the term “alkynyl” includes straight-chain alkynyl groups (e.g., ethynyl, propynyl, butynyl, pentynyl, hexynyl, heptynyl, octynyl, nonynyl, decynyl, etc.), branched-chain alkynyl groups, and cycloalkyl or cycloalkenyl substituted alkynyl groups. The term alkynyl further includes alkynyl groups that include oxygen, nitrogen, sulfur or phosphorous atoms replacing one or more carbons of the hydrocarbon backbone. In certain embodiments; a straight chain or branched chain alkynyl group has 6 or fewer carbon atoms in its backbone (e.g., C₂-C₆ for straight chain, C₃-C₆ for branched chain). The term C₂-C₆ includes alkynyl groups containing 2 to 6 carbon atoms.

Moreover, the term alkynyl includes both “unsubstituted alkynyls” and “substituted alkynyls”, the latter of which refers to alkynyl moieties having substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “amine” or “amino” should be understood as being broadly applied to both a molecule, or a moiety or functional group, as generally understood in the art, and may be primary, secondary, or tertiary. The term “amine” or “amino” includes compounds where a nitrogen atom is covalently bonded to at least one carbon, hydrogen or heteroatom. The terms include, for example, but are not limited to, “alkylamino,” “arylamino,” “diarylamino,” “alkylarylamino,” “alkylaminoaryl,” “arylaminoalkyl,” “alkaminoalkyl,” “amide,” “amido,” and “aminocarbonyl.” The term “alkyl amino” comprises groups and compounds wherein the nitrogen is bound to at least one additional alkyl group. The term “dialkyl amino” includes groups wherein the nitrogen atom is bound to at least two additional alkyl groups. The term “arylamino” and “diarylamino” include groups wherein the nitrogen is bound to at least one or two aryl groups, respectively. The term “alkylarylamino,” “alkylaminoaryl” or “arylaminoalkyl” refers to an amino group which is bound to at least one alkyl group and at least one aryl group. The term “alkaminoalkyl” refers to an alkyl, alkenyl, or alkynyl group bound to a nitrogen atom which is also bound to an alkyl group.

The term “amide,” “amido” or “aminocarbonyl” includes compounds or moieties which contain a nitrogen atom which is bound to the carbon of a carbonyl or a thiocarbonyl group. The term includes “alkaminocarbonyl” or “alkylaminocarbonyl” groups which include alkyl, alkenyl, aryl or alkynyl groups bound to an amino group bound to a carbonyl group. It includes arylaminocarbonyl and arylcarbonylamino groups which include aryl or heteroaryl moieties bound to an amino group which is bound to the carbon of a carbonyl or thiocarbonyl group. The terms “alkylaminocarbonyl,” “alkenylaminocarbonyl,” “alkynylaminocarbonyl,” “arylaminocarbonyl,” “alkylcarbonylamino,” “alkenylcarbonylamino,” “alkynylcarbonylamino,” and “arylcarbonylamino” are included in term “amide.” Amides also include urea groups (aminocarbonylamino) and carbamates (oxycarbonylamino).

The term “aryl” includes groups, including 5- and 6-membered single-ring aromatic groups that may include from zero to four heteroatoms, for example, phenyl, pyrrole, furan, thiophene, thiazole, isothiaozole, imidazole, triazole, tetrazole, pyrazole, oxazole, isoxazole, pyridine, pyrazine, pyridazine, and pyrimidine, and the like. Furthermore, the term “aryl” includes multicyclic aryl groups, e.g., tricyclic, bicyclic, e.g., naphthalene, benzoxazole, benzodioxazole, benzothiazole, benzoimidazole, benzothiophene, methylenedioxyphenyl, quinoline, isoquinoline, anthryl, phenanthryl, napthridine, indole, benzofuran, purine, benzofuran, deazapurine, or indolizine. Those aryl groups having heteroatoms in the ring structure may also be referred to as “aryl heterocycles”, “heterocycles,” “heteroaryls” or “heteroaromatics.” The aromatic ring can be substituted at one or more ring positions with such substituents as described above, as for example, alkyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminoacarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

Certain aryl groups recited herein are C₆-C₁₀arylC₀-C₈alkyl groups (i.e., groups in which a 6- to 10-membered carbocyclic group comprising at least one aromatic ring is linked via a single covalent bond or a C₁-C₈alkylene group). Such groups include, for example, phenyl and indanyl, as well as groups in which either of the foregoing is linked via C₁-C₈alkylene, preferably via C₁-C₄alkylene. Phenyl groups linked via a single covalent bond or C₁-C₆alkylene group are designated phenylC₀-C₆alkyl (e.g., benzyl, 1-phenyl-ethyl, 1-phenyl-propyl and 2-phenyl-ethyl).

“Arylene” refers to a divalent aryl group, as defined above. Arylene is intended to encompass divalent residues of phenyl, naphthyl and biphenyl. “Heteroarylene” refers to divalent heteroaryl groups as defined infra.

The term “heteroaryl”, as used herein, represents a stable monocyclic or bicyclic ring of up to 7 atoms in each ring, wherein at least one ring is aromatic and contains from 1 to 4 heteroatoms selected from the group consisting of O, N and S. Heteroaryl groups within the scope of this definition include but are not limited to: acridinyl, carbazolyl, cinnolinyl, quinoxalinyl, pyrrazolyl, indolyl, isoindoline, benzotriazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetrahydroquinoline. As with the definition of heterocycle below, “heteroaryl” is also understood to include the N-oxide derivative of any nitrogen-containing heteroaryl. In cases where the heteroaryl substituent is bicyclic and one ring is non-aromatic or contains no heteroatoms, it is understood that attachment is via the aromatic ring or via the heteroatom containing ring, respectively.

The term “heterocycle” or “heterocyclyl” as used herein is intended to mean a 5- to 10-membered aromatic or nonaromatic heterocycle containing from 1 to 4 heteroatoms selected from the group consisting of O, N and S, and includes bicyclic groups. “Heterocyclyl” therefore includes the above mentioned heteroaryls, as well as dihydro and tetrathydro analogs thereof. Further examples of “heterocyclyl” include, but are not limited to the following: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydropyranyl, tetrazolyl, tetrazolopyridyl, thiadiazolyl, thiazolyl, thienyl, triazolyl, azetidinyl, 1,4-dioxanyl, hexahydroazepinyl, piperazinyl, piperidinyl, pyridin-2-onyl, pyrrolidinyl, morpholinyl, thiomorpholinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl, and N-oxides thereof. Attachment of a heterocyclyl substituent can occur via a carbon atom or via a heteroatom.

A “heterocycleC₀-C₈alkyl” is a heterocyclic group linked via a single covalent bond or C₁-C₈alkylene group. A (4- to 7-membered heterocycle)C₀-C₈alkyl is a heterocyclic group (e.g., monocyclic or bicyclic) having from 4 to 7 ring members linked via a single covalent bond or an alkylene group having from 1 to 8 carbon atoms. A “(6-membered heteroaryl)C₀-C₆alkyl” refers to a heteroaryl group linked via a direct bond or C₁-C₆alkyl group.

The term “acyl” includes compounds and moieties which contain the acyl radical (CH₃CO—) or a carbonyl group. The term “substituted acyl” includes acyl groups where one or more of the hydrogen atoms are replaced by for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “acylamino” includes moieties wherein an acyl moiety is bonded to an amino group. For example, the term includes alkylcarbonylamino, arylcarbonylamino, carbamoyl and ureido groups.

The term “alkoxy” includes substituted and unsubstituted alkyl, alkenyl, and alkynyl groups covalently linked to an oxygen atom. Examples of alkoxy groups include methoxy, ethoxy, isopropyloxy, propoxy, butoxy, and pentoxy groups and may include cyclic groups such as cyclopentoxy. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy, trichloromethoxy, etc.

The term “carbonyl” or “carboxy” includes compounds and moieties which contain a carbon connected with a double bond to an oxygen atom, and tautomeric forms thereof. Examples of moieties that contain a carbonyl include aldehydes, ketones, carboxylic acids, amides, esters, anhydrides, etc. The term “carboxy moiety” or “carbonyl moiety” refers to groups such as “alkylcarbonyl” groups wherein an alkyl group is covalently bound to a carbonyl group, “alkenylcarbonyl” groups wherein an alkenyl group is covalently bound to a carbonyl group, “alkynylcarbonyl” groups wherein an alkynyl group is covalently bound to a carbonyl group, “arylcarbonyl” groups wherein an aryl group is covalently attached to the carbonyl group. Furthermore, the term also refers to groups wherein one or more heteroatoms are covalently bonded to the carbonyl moiety. For example, the term includes moieties such as, for example, aminocarbonyl moieties, (wherein a nitrogen atom is bound to the carbon of the carbonyl group, e.g., an amide), aminocarbonyloxy moieties, wherein an oxygen and a nitrogen atom are both bond to the carbon of the carbonyl group (e.g., also referred to as a “carbamate”). Furthermore, aminocarbonylamino groups (e.g., ureas) are also include as well as other combinations of carbonyl groups bound to heteroatoms (e.g., nitrogen, oxygen, sulfur, etc. as well as carbon atoms). Furthermore, the heteroatom can be further substituted with one or more alkyl, alkenyl, alkynyl, aryl, aralkyl, acyl, etc. moieties.

The term “thiocarbonyl” or “thiocarboxy” includes compounds and moieties which contain a carbon connected with a double bond to a sulfur atom. The term “thiocarbonyl moiety” includes moieties that are analogous to carbonyl moieties. For example, “thiocarbonyl” moieties include aminothiocarbonyl, wherein an amino group is bound to the carbon atom of the thiocarbonyl group, furthermore other thiocarbonyl moieties include, oxythiocarbonyls (oxygen bound to the carbon atom), aminothiocarbonylamino groups, etc.

The term “ether” includes compounds or moieties that contain an oxygen bonded to two different carbon atoms or heteroatoms. For example, the term includes “alkoxyalkyl” which refers to an alkyl, alkenyl, or alkynyl group covalently bonded to an oxygen atom that is covalently bonded to another alkyl group.

The term “ester” includes compounds and moieties that contain a carbon or a heteroatom bound to an oxygen atom that is bonded to the carbon of a carbonyl group. The term “ester” includes alkoxycarboxy groups such, as methoxycarbonyl, ethoxycarbonyl, propoxycarbonyl, butoxycarbonyl, pentoxycarbonyl, etc. The alkyl, alkenyl, or alkynyl groups are as defined above.

The term “thioether” includes compounds and moieties which contain a sulfur atom bonded to two different carbon or hetero atoms. Examples of thioethers include, but are not limited to alkthioalkyls, alkthioalkenyls, and alkthioalkynyls. The term “alkthioalkyls” include compounds with an alkyl, alkenyl, or alkynyl group bonded to a sulfur atom that is bonded to an alkyl group. Similarly, the term “alkthioalkenyls” and alkthioalkynyls” refer to compounds or moieties wherein an alkyl, alkenyl, or alkynyl group is bonded to a sulfur atom which is covalently bonded to an alkynyl group.

The term “hydroxy” or “hydroxyl” includes groups with an —OH or —O_.

The term “halogen” includes fluorine, bromine, chlorine, iodine, etc. The term “perhalogenated” generally refers to a moiety wherein all hydrogens are replaced by halogen atoms.

The terms “polycyclyl” or “polycyclic radical” include moieties with two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, alkylaminoacarbonyl, aralkylaminocarbonyl, alkenylaminocarbonyl, alkylcarbonyl, arylcarbonyl, aralkylcarbonyl, alkenylcarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkyl, alkylaryl, or an aromatic or heteroaromatic moiety.

The term “heteroatom” includes atoms of any element other than carbon or hydrogen. Preferred heteroatoms are nitrogen, oxygen, sulfur and phosphorus.

Additionally, the phrase “any combination thereof” implies that any number of the listed functional groups and molecules may be combined to create a larger molecular architecture. For example, the terms “phenyl,” “carbonyl” (or “═O”), “—O—,” “—OH,” and C₁₋₆ (i.e., —CH₃ and —CH₂CH₂CH₂—) can be combined to form a 3-methoxy-4-propoxybenzoic acid substituent. It is to be understood that when combining functional groups and molecules to create a larger molecular architecture, hydrogens can be removed or added, as required to satisfy the valence of each atom.

It is to be understood that all of the compounds of the invention described above will further include bonds between adjacent atoms and/or hydrogens as required to satisfy the valence of each atom. That is, bonds and/or hydrogen atoms are added to provide the following number of total bonds to each of the following types of atoms: carbon: four bonds; nitrogen: three bonds; oxygen: two bonds; and sulfur: two bonds.

Groups that are “optionally substituted” are unsubstituted or are substituted by other than hydrogen at one or more available positions, typically 1, 2, 3, 4 or 5 positions, by one or more suitable groups (which may be the same or different). Optional substitution is also indicated by the phrase “substituted with from 0 to X substituents,” where X is the maximum number of possible substituents. Certain optionally substituted groups are substituted with from 0 to 2, 3 or 4 independently selected substituents (i.e., are unsubstituted or substituted with up to the recited maximum number of substitutents).

It will be noted that the structures of some of the compounds of this invention include asymmetric carbon atoms. It is to be understood accordingly that the isomers arising from such asymmetry (e.g., all enantiomers, stereoisomers, rotamers, tautomers, diastereomers, or racemates) are included within the scope of this invention. Such isomers can be obtained in substantially pure form by classical separation techniques and by stereochemically controlled synthesis. Furthermore, the structures and other compounds and moieties discussed in this application also include all tautomers thereof. Compounds described herein may be obtained through art recognized synthesis strategies.

It will also be noted that the substituents of some of the compounds of this invention include isomeric cyclic structures. It is to be understood accordingly that constitutional isomers of particular substituents are included within the scope of this invention, unless indicated otherwise. For example, the term “tetrazole” includes tetrazole, 2H-tetrazole, 3H-tetrazole, 4H-tetrazole and 5H-tetrazole.

### Use in HCV-Associated Disorders

The compounds of the present invention have valuable pharmacological properties and are useful in the treatment of diseases. In certain embodiments, compounds of the invention are useful in the treatment of HCV-associated disorders, e.g., as drugs to treat HCV infection.

The term “use” includes any one or more of the following embodiments of the invention, respectively: the use in the treatment of HCV-associated disorders; the use for the manufacture of pharmaceutical compositions for use in the treatment of these diseases, e.g., in the manufacture of a medicament; methods of use of compounds of the invention in the treatment of these diseases; pharmaceutical preparations having compounds of the invention for the treatment of these diseases; and compounds of the invention for use in the treatment of these diseases; as appropriate and expedient, if not stated otherwise. In particular, diseases to be treated and are thus preferred for use of a compound of the present invention are selected from HCV-associated disorders, including those corresponding to HCV-infection, as well as those diseases that depend on the activity of one or more of the NS3, NS4A, NS4B, NS5A and NS5B proteins, or a NS3-NS4A, NS4A-NS4B, NS4B-NS5A or NS5A-NS5B complex. The term “use” further includes embodiments of compositions herein which bind to an HCV protein sufficiently to serve as tracers or labels, so that when coupled to a fluor or tag, or made radioactive, can be used as a research reagent or as a diagnostic or an imaging agent.

In certain embodiments, a compound of the present invention is used for treating HCV-associated diseases, and use of the compound of the present invention as an inhibitor of any one or more HCVs. It is envisioned that a use can be a treatment of inhibiting one or more strains of HCV.

### Assays

The inhibition of HCV activity may be measured as using a number of assays available in the art. An example of such an assay can be found in Anal Biochem. 1996 240(1): 60-7; which is incorporated by reference in its entirety. Assays for measurement of HCV activity are also described in the experimental section below.

### Pharmaceutical Compositions

The language “effective amount” of the compound is that amount necessary or sufficient to treat or prevent an HCV-associated disorder, e.g. prevent the various morphological and somatic symptoms of an HCV-associated disorder, and/or a disease or condition described herein. In an example, an effective amount of the HCV-modulating compound is the amount sufficient to treat HCV infection in a subject. In another example, an effective amount of the HCV-modulating compound is the amount sufficient to treat HCV infection, liver cirrhosis, chronic liver disease, hepatocellular carcinoma, cryoglobulinaemia, non-Hodgkin's lymphoma, and a suppressed innate intracellular immune response in a subject. The effective amount can vary depending on such factors as the size and weight of the subject, the type of illness, or the particular compound of the invention. For example, the choice of the compound of the invention can affect what constitutes an “effective amount.” One of ordinary skill in the art would be able to study the factors contained herein and make the determination regarding the effective amount of the compounds of the invention without undue experimentation.

The regimen of administration can affect what constitutes an effective amount. The compound of the invention can be administered to the subject either prior to or after the onset of an HCV-associated state. Further, several divided dosages, as well as staggered dosages, can be administered daily or sequentially, or the dose can be continuously infused, or can be a bolus injection. Further, the dosages of the compound(s) of the invention can be proportionally increased or decreased as indicated by the exigencies of the therapeutic or prophylactic situation.

Compounds of the invention may be used in the treatment of states, disorders or diseases as described herein, or for the manufacture of pharmaceutical compositions for use in the treatment of these diseases. Methods of use of compounds of the present invention in the treatment of these diseases, or pharmaceutical preparations having compounds of the present invention for the treatment of these diseases.

The language “pharmaceutical composition” includes preparations suitable for administration to mammals, e.g., humans. When the compounds of the present invention are administered as pharmaceuticals to mammals, e.g., humans, they can be given per se or as a pharmaceutical composition containing, for example, 0.1 to 99.5% (more preferably, 0.5 to 90%) of active ingredient in combination with a pharmaceutically acceptable carrier.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethyleneglycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, α-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral, nasal, topical, transdermal, buccal, sublingual, rectal, vaginal and/or parenteral administration. The formulations may conveniently be presented in unit dosage form and may be prepared by any methods well known in the art of pharmacy. The amount of active ingredient that can be combined with a carrier material to produce a single dosage form will generally be that amount of the compound that produces a therapeutic effect. Generally, out of one hundred per cent, this amount will range from about 1 per cent to about ninety-nine percent of active ingredient, preferably from about 5 per cent to about 70 per cent, most preferably from about 10 per cent to about 30 per cent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluent commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

The preparations of the present invention may be given orally, parenterally, topically, or rectally. They are of course given by forms suitable for each administration route. For example, they are administered in tablets or capsule form, by injection, inhalation, eye lotion, ointment, suppository, etc., administration by injection, infusion or inhalation; topical by lotion or ointment; and rectal by suppositories. Oral administration is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound that is the lowest dose effective to produce a therapeutic effect. Such an effective dose will generally depend upon the factors described above. Generally, intravenous and subcutaneous doses of the compounds of this invention for a patient, when used for the indicated analgesic effects, will range from about 0.0001 to about 100 mg per kilogram of body weight per day, more preferably from about 0.01 to about 50 mg per kg per day, and still more preferably from about 1.0 to about 100 mg per kg per day. An effective amount is that amount treats an HCV-associated disorder.

If desired, the effective daily dose of the active compound may be administered as two, three, four, five, six or more sub-doses administered separately at appropriate intervals throughout the day, optionally, in unit dosage forms.

While it is possible for a compound of the present invention to be administered alone, it is preferable to administer the compound as a pharmaceutical composition.

### Synthetic Procedure

Compounds of the present invention are prepared from commonly available compounds using procedures known to those skilled in the art, including any one or more of the following conditions without limitation:

Within the scope of this text, only a readily removable group that is not a constituent of the particular desired end product of the compounds of the present invention is designated a “protecting group,” unless the context indicates otherwise. The protection of functional groups by such protecting groups, the protecting groups themselves, and their cleavage reactions are described for example in standard reference works, such as e.g., Science of Synthesis: Houben-Weyl Methods of Molecular Transformation. Georg Thieme Verlag, Stuttgart, Germany. 2005. 41627 pp. (URL: http://www.science-of-synthesis.com (Electronic Version, 48 Volumes)); J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, N.Y. 1999, in “The Peptides”; Volume 3 (editors: E. Gross and J. Meienhofer), Academic Press, London and New York 1981, in “Methoden der organischen Chemie” (Methods of Organic Chemistry), Houben Weyl, 4th edition, Volume 15/I, Georg Thieme Verlag, Stuttgart 1974, in H.-D. Jakubke and H. Jeschkeit, “Aminosäuren, Peptide, Proteine” (Amino acids, Peptides, Proteins), Verlag Chemie, Weinheim, Deerfield Beach, and Basel 1982, and in Jochen Lehmann, “Chemie der Kohlenhydrate: Monosaccharide and Derivate” (Chemistry of Carbohydrates: Monosaccharides and Derivatives), Georg Thieme Verlag, Stuttgart 1974. A characteristic of protecting groups is that they can be removed readily (i.e., without the occurrence of undesired secondary reactions) for example by solvolysis, reduction, photolysis or alternatively under physiological conditions (e.g., by enzymatic cleavage).

Salts of compounds of the present invention having at least one salt-forming group may be prepared in a manner known per se. For example, salts of compounds of the present invention having acid groups may be formed, for example, by treating the compounds with metal compounds, such as alkali metal salts of suitable organic carboxylic acids, e.g., the sodium salt of 2-ethylhexanoic acid, with organic alkali metal or alkaline earth metal compounds, such as the corresponding hydroxides, carbonates or hydrogen carbonates, such as sodium or potassium hydroxide, carbonate or hydrogen carbonate, with corresponding calcium compounds or with ammonia or a suitable organic amine, stoichiometric amounts or only a small excess of the salt-forming agent preferably being used. Acid addition salts of compounds of the present invention are obtained in customary manner, e.g., by treating the compounds with an acid or a suitable anion exchange reagent. Internal salts of compounds of the present invention containing acid and basic salt-forming groups, e.g., a free carboxy group and a free amino group, may be formed, e.g., by the neutralisation of salts, such as acid addition salts, to the isoelectric point, e.g., with weak bases, or by treatment with ion exchangers. Salts can be converted in customary manner into the free compounds; metal and ammonium salts can be converted, for example, by treatment with suitable acids, and acid addition salts, for example, by treatment with a suitable basic agent.

Mixtures of isomers obtainable according to the invention can be separated in a manner known per se into the individual isomers; diastereoisomers can be separated, for example, by partitioning between polyphasic solvent mixtures, recrystallisation and/or chromatographic separation, for example over silica gel or by, e.g., medium pressure liquid chromatography over a reversed phase column, and racemates can be separated, for example, by the formation of salts with optically pure salt-forming reagents and separation of the mixture of diastereoisomers so obtainable, for example by means of fractional crystallisation, or by chromatography over optically active column materials.

Intermediates and final products can be worked up and/or purified according to standard methods, e.g., using chromatographic methods, distribution methods, (re-) crystallization, and the like.

### General Process Conditions

The following applies in general to all processes mentioned throughout this disclosure.

The process steps to synthesize the compounds of the invention can be carried out under reaction conditions that are known per se, including those mentioned specifically, in the absence or, customarily, in the presence of solvents or diluents, including, for example, solvents or diluents that are inert towards the reagents used and dissolve them, in the absence or presence of catalysts, condensation or neutralizing agents, for example ion exchangers, such as cation exchangers, e.g., in the H₊ form, depending on the nature of the reaction and/or of the reactants at reduced, normal or elevated temperature, for example in a temperature range of from about −100° C. to about 190° C., including, for example, from approximately −80° C. to approximately 150° C., for example at from −80 to −60° C., at room temperature, at from −20 to 40° C. or at reflux temperature, under atmospheric pressure or in a closed vessel, where appropriate under pressure, and/or in an inert atmosphere, for example under an argon or nitrogen atmosphere.

At all stages of the reactions, mixtures of isomers that are formed can be separated into the individual isomers, for example diastereoisomers or enantiomers, or into any desired mixtures of isomers, for example racemates or mixtures of diastereoisomers, for example analogously to the methods described in Science of Synthesis: Houben-Weyl Methods of Molecular Transformation. Georg Thieme Verlag, Stuttgart, Germany. 2005.

The solvents from which those solvents that are suitable for any particular reaction may be selected include those mentioned specifically or, for example, water, esters, such as lower alkyl-lower alkanoates, for example ethyl acetate, ethers, such as aliphatic ethers, for example diethyl ether, or cyclic ethers, for example tetrahydrofurane or dioxane, liquid aromatic hydrocarbons, such as benzene or toluene, alcohols, such as methanol, ethanol or 1- or 2-propanol, nitriles, such as acetonitrile, halogenated hydrocarbons, such as methylene chloride or chloroform, acid amides, such as dimethylformamide or dimethyl acetamide, bases, such as heterocyclic nitrogen bases, for example pyridine or N-methylpyrrolidin-2-one, carboxylic acid anhydrides, such as lower alkanoic acid anhydrides, for example acetic anhydride, cyclic, linear or branched hydrocarbons, such as cyclohexane, hexane or isopentane, or mixtures of those solvents, for example aqueous solutions, unless otherwise indicated in the description of the processes. Such solvent mixtures may also be used in working up, for example by chromatography or partitioning.

The compounds, including their salts, may also be obtained in the form of hydrates, or their crystals may, for example, include the solvent used for crystallization. Different crystalline forms may be present.

The invention relates also to those forms of the process in which a compound obtainable as an intermediate at any stage of the process is used as starting material and the remaining process steps are carried out, or in which a starting material is formed under the reaction conditions or is used in the form of a derivative, for example in a protected form or in the form of a salt, or a compound obtainable by the process according to the invention is produced under the process conditions and processed further in situ.

### Pro-Drugs

The present invention also relates to pro-drugs of a compound of the present invention that are converted in vivo to the compounds of the present invention as described herein. Any reference to a compound of the present invention is therefore to be understood as referring also to the corresponding pro-drugs of the compound of the present invention, as appropriate and expedient.

### Combinations

A compound of the present invention may also be used in combination with other agents, e.g., an additional HCV-modulating compound that is or is not of the formula I, for treatment of and HCV-associated disorder in a subject.

By the term “combination”, is meant either a fixed combination in one dosage unit form, or a kit of parts for the combined administration where a compound of the present invention and a combination partner may be administered independently at the same time or separately within time intervals that especially allow that the combination partners show a cooperative, e.g., synergistic, effect, or any combination thereof.

For example, WO 2005/042020, incorporated herein by reference in its entirety, describes the combination of various HCV inhibitors with a cytochrome P450 (“CYP”) inhibitor. Any CYP inhibitor that improves the pharmacokinetics of the relevant NS3/4A protease may be used in combination with the compounds of this invention. These CYP inhibitors include, but are not limited to, ritonavir (WO 94/14436, incorporated herein by reference in its entirety), ketoconazole, troleandomycin, 4-methyl pyrazole, cyclosporin, clomethiazole, cimetidine, itraconazole, fluconazole, miconazole, fluvoxamine, fluoxetine, nefazodone, sertraline, indinavir, nelfinavir, amprenavir, fosamprenavir, saquinavir, lopinavir, delavirdine, erythromycin, VX-944, and VX-497. Preferred CYP inhibitors include ritonavir, ketoconazole, troleandomycin, 4-methyl pyrazole, cyclosporin, and clomethiazole.

Methods for measuring the ability of a compound to inhibit CYP activity are known (see, e.g., U.S. Pat. No. 6,037,157 and Yun, et al. Drug Metabolism & Disposition, vol. 21, pp. 403-407 (1993); incorporated herein by reference). For example, a compound to be evaluated may be incubated with 0.1, 0.5, and 1.0 mg protein/ml, or other appropriate concentration of human hepatic microsomes (e. g., commercially available, pooled characterized hepatic microsomes) for 0, 5, 10, 20, and 30 minutes, or other appropriate times, in the presence of an NADPH-generating system. Control incubations may be performed in the absence of hepatic microsomes for 0 and 30 minutes (triplicate). The samples may be analyzed for the presence of the compound. Incubation conditions that produce a linear rate of compound metabolism will be used a guide for further studies. Experiments known in the art can be used to determine the kinetics of the compound metabolism (Kₘ and Vₘₐₓ). The rate of disappearance of compound may be determined and the data analyzed according to Michaelis-Menten kinetics by using Lineweaver-Burk, Eadie-Hofstee, or nonlinear regression analysis.

Inhibition of metabolism experiments may then be performed. For example, a compound (one concentration, ≦Kₘ) may be incubated with pooled human hepatic microsomes in the absence or presence of a CYP inhibitor (such as ritonavir) under the conditions determined above. As would be recognized, control incubations should contain the same concentration of organic solvent as the incubations with the CYP inhibitor. The concentrations of the compound in the samples may be quantitated, and the rate of disappearance of parent compound may be determined, with rates being expressed as a percentage of control activity.

Methods for evaluating the influence of co-administration of a compound of the invention and a CYP inhibitor in a subject are also known (see, e.g., US2004/0028755; incorporated herein by reference). Any such methods could be used in connection with this invention to determine the pharmacokinetic impact of a combination. Subjects that would benefit from treatment according to this invention could then be selected.

Accordingly, one embodiment of this invention provides a method for administering an inhibitor of CYP3A4 and a compound of the invention. Another embodiment of this invention provides a method for administering an inhibitor of isozyme 3A4 (“CYP3A4”), isozyme 2C19 (“CYP2C19”), isozyme 2D6 (“CYP2D6”), isozyme 1A2 (“CYP1A2”), isozyme 2C9 (“CYP2C9”), or isozyme 2E1 (“CYP2E1”). In embodiments where the protease inhibitor is VX-950 (or a sterereoisomer thereof), the CYP inhibitor preferably inhibits CYP3A4.

As would be appreciated, CYP3A4 activity is broadly observed in humans. Accordingly, embodiments of this invention involving inhibition of isozyme 3A4 would be expected to be applicable to a broad range of patients.

Accordingly, this invention provides methods wherein the CYP inhibitor is administered together with the compound of the invention in the same dosage form or in separate dosage forms.

The compounds of the invention (e.g., compound of Formula I or subformulae thereof) may be administered as the sole ingredient or in combination or alteration with other antiviral agents, especially agents active against HCV. In combination therapy, effective dosages of two or more agents are administered together, whereas in alternation or sequential-step therapy, an effective dosage of each agent is administered serially or sequentially. In general, combination therapy is typically preferred over alternation therapy because it induces multiple simultaneous stresses on the virus. The dosages given will depend on absorption, inactivation and excretion rate of the drug as well as other factors. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions. The efficacy of a drug against the viral infection can be prolonged, augmented, or restored by administering the compound in combination or alternation with a second, and perhaps third antiviral compound that induces a different gene mutation than that caused by the principle drug in a drug resistant virus. Alternatively, the pharmacokinetic, biodistribution or other parameters of the drug can be altered by such combination or alternation therapy.

Daily dosages required in practicing the method of the present invention will vary depending upon, for example, the compound of the invention employed, the host, the mode of administration, the severity of the condition to be treated. A preferred daily dosage range is about from 1 to 50 mg/kg per day as a single dose or in divided doses. Suitable daily dosages for patients are on the order of from e.g. 1 to 20 mg/kg p.o or i.v. Suitable unit dosage forms for oral administration comprise from ca. 0.25 to 10 mg/kg active ingredient, e.g. compound of Formula I or any subformulae thereof, together with one or more pharmaceutically acceptable diluents or carriers therefor. The amount of co-agent in the dosage form can vary greatly, e.g., 0.00001 to 1000mg/kg active ingredient.

Daily dosages with respect to the co-agent used will vary depending upon, for example, the compound employed, the host, the mode of administration and the severity of the condition to be treated. For example, lamivudine may be administered at a daily dosage of 100mg. The pegylated interferon may be administered parenterally one to three times per week, preferably once a week, at a total weekly dose ranging from 2 to 10 million IU, more preferable 5 to 10 million IU, most preferable 8 to 10 million IU. Because of the diverse types of co-agent that may be used, the amounts can vary greatly, e.g., 0.0001 to 5,000 mg/kg per day.

The current standard of care for treating hepatitis C is the combination of pegylated interferon alpha with ribavirin, of which the recommended doses are1.5 μg/kg/wk peginterferon alfa-2b or 180 μg/wk peginterferon alfa-2a, plus 1,000 to 1,200 mg daily of ribavirin for 48 weeks for genotype I patients, or 800 mg daily of ribavirin for 24 weeks for genotype 2/3 patients.

The compound of the invention (e.g., compound of Formula I or subformulae thereof) and co-agents of the invention may be administered by any conventional route, in particular enterally, e.g. orally, for example in the form of solutions for drinking, tablets or capsules or parenterally, for example in the form of injectable solutions or suspensions. Certain preferred pharmaceutical compositions may be e.g. those based on microemulsions as described in UK 2,222,770 A.

The compound of the invention (e.g., compound of Formula I or subformulae thereof) are administered together with other drugs (co-agents) e.g. a drug which has anti-viral activity, especially anti-Flaviviridae activity, most especially anti-HCV activity, e.g. an interferon, e.g. interferon-α-2a or interferon-α-2b, e.g. Intron® A, Roferon®, Avonex®, Rebif® or Betaferon®, or an interferon conjugated to a water soluble polymer or to human albumin, e.g. albuferon, an anti-viral agent, e.g. ribavirin, lamivudine, the compounds disclosed in U.S. Pat. No. 6,812,219 and WO 2004/002422 A2 (the disclosures of which are incorporated herein by reference in their entireties), an inhibitor of the HCV or other Flaviviridae virus encoded factors like the NS3/4A protease, helicase or RNA polymerase or a prodrug of such an inhibitor, an anti-fibrotic agent, e.g. a N-phenyl-2-pyrimidine-amine derivative, e.g. imatinib, an immune modulating agent, e.g. mycophenolic acid, a salt or a prodrug thereof, e.g. sodium mycophenolate or mycophenolate mofetil, or a SIP receptor agonist, e.g. FTY720 or an analogue thereof optionally phosphorylated, e.g. as disclosed in EP627406A1, EP778263A1, EP1002792A1, WO02/18395, WO02/76995, WO 02/06268, JP2002316985, WO03/29184, WO03/29205, WO03/62252 and WO03/62248, the disclosures of which are incorporated herein by reference in their entireties.

Conjugates of interferon to a water-soluble polymer are meant to include especially conjugates to polyalkylene oxide homopolymers such as polyethylene glycol (PEG) or polypropylene glycols, polyoxyethylenated polyols, copolymers thereof and block copolymers thereof. As an alternative to polyalkylene oxide-based polymers, effectively non-antigenic materials such as dextran, polyvinyl pyrrolidones, polyacrylamides, polyvinyl alcohols, carbohydrate-based polymers and the like can be used. Such interferon-polymer conjugates are described in U.S. Pat. Nos. 4,766,106, 4,917,888, European Patent Application No. 0 236 987, European Patent Application No. 0 510 356 and International Application Publication No. WO 95/13090, the disclosures of which are incorporated herein by reference in their entireties. Since the polymeric modification sufficiently reduces antigenic responses, the foreign interferon need not be completely autologous. Interferon used to prepare polymer conjugates may be prepared from a mammalian extract, such as human, ruminant or bovine interferon, or recombinantly produced. Preferred are conjugates of interferon to polyethylene glycol, also known as pegylated interferons.

Especially preferred conjugates of interferon are pegylated alfa-interferons, for example pegylated interferon-α-2a, pegylated interferon-α-2b; pegylated consensus interferon or pegylated purified interferon-a product. Pegylated interferon-α-2a is described e.g. in European Patent 593,868 (incorporated herein by reference in its entirety) and commercially available e. g. under the tradename PEGASYS® (Hoffmann-La Roche). Pegylated interferon-α-2b is described, e.g. in European Patent 975,369 (incorporated herein by reference in its entirety) and commercially available e.g. under the tradename PEG-INTRON A® (Schering Plough). Pegylated consensus interferon is described in WO 96/11953 (incorporated herein by reference in its entirety). The preferred pegylated α-interferons are pegylated interferon-α-2a and pegylated interferon-α-2b. Also preferred is pegylated consensus interferon.

Other preferred co-agents are fusion proteins of an interferon, for example fusion proteins of interferon-α-2a, interferon-α-2b; consensus interferon or purified interferon-α product, each of which is fused with another protein. Certain preferred fusion proteins comprise an interferon (e.g., interferon-α-2b) and an albumin as described in U.S. Pat. No. 6,973,322 and international publications WO02/60071, WO05/003296 and WO05/077042 (Human Genome Sciences). A preferred interferon conjugated to a human albumin is Albuferon (Human Genome Sciences).

Cyclosporins which bind strongly to cyclophilin but are not immunosuppressive include those cyclosporins recited in U.S. Pat. Nos. 5,767,069 and 5,981,479 and are incorporated herein by reference. MeIle⁴-Cyclosporin is a preferred non-immunosuppressive cyclosporin. Certain other cyclosporin derivatives are described in WO2006039668 (Scynexis) and WO2006038088 (Debiopharm SA) and are incorporated herein by reference. A cyclosporin is considered to be non-immunosuppressive when it has an activity in the Mixed Lymphocyte Reaction (MLR) of no more than 5%, preferably no more than 2%, that of cyclosporin A. The Mixed Lymphocyte Reaction is described by T. Meo in “Immunological Methods”, L. Lefkovits and B. Peris, Eds., Academic Press, N.Y. pp. 227-239 (1979). Spleen cells (0.5×10⁶) from Balb/c mice (female, 8-10 weeks) are co-incubated for 5 days with 0.5×10⁶ irradiated (2000 rads) or mitomycin C treated spleen cells from CBA mice (female, 8-10 weeks). The irradiated allogeneic cells induce a proliferative response in the Balb c spleen cells which can be measured by labeled precursor incorporation into the DNA. Since the stimulator cells are irradiated (or mitomycin C treated) they do not respond to the Balb/c cells with proliferation but do retain their antigenicity. The IC₅₀ found for the test compound in the MLR is compared with that found for cyclosporin A in a parallel experiment. In addition, non-immunosuppressive cyclosporins lack the capacity of inhibiting CN and the downstream NF-AT pathway. [MeIle]⁴-ciclosporin is a preferred non-immunosuppressive cyclophilin-binding cyclosporin for use according to the invention.

Ribavirin (1-β-D-ribofuranosyl-1-1,2,4-triazole-3-caroxamide) is a synthetic, non-interferon-inducing, broad spectrum antiviral nucleoside analog sold under the trade name, Virazole (The Merk Index, 11^{th} edition, Editor: Budavar, S, Merck & Co., Inc., Rahway, N.J., p 1304, 1989). U.S. Pat. Nos. 3,798,209 and RE29,835 (incorporated herein by reference in their entireties) disclose and claim ribavirin. Ribavirin is structurally similar to guanosine, and has in vitro activity against several DNA and RNA viruses including Flaviviridae (Gary L. Davis, Gastroenterology 118:S104-S114, 2000).

Ribavirin reduces serum amino transferase levels to normal in 40% of patients, but it does not lower serum levels of HCV-RNA (Gary L. Davis, Gastroenterology 118:S104-S114, 2000). Thus, ribavirin alone is not effective in reducing viral RNA levels. Additionally, ribavirin has significant toxicity and is known to induce anemia. Ribavirin is not approved for monotherapy against HCV; it is approved in combination with interferon alpha-2a or interferon alpha-2b for the treatment of HCV.

A further preferred combination is a combination of a compound of the invention (e.g., a compound of Formula I or any subformulae thereof) with a non-immunosuppressive cyclophilin-binding cyclosporine, with mycophenolic acid, a salt or a prodrug thereof, and/or with a SIP receptor agonist, e.g. FTY720.

Additional examples of compounds that can be used in combination or alternation treatments include:

(1) Interferons, including interferon alpha 2a or 2b and pegylated (PEG) interferon alpha 2a or 2b, for example:
(a) Intron-A®, interferon alfa-2b (Schering Corporation, Kenilworth, N.J.);
(b) PEG-Intron®, peginteferon alfa-2b (Schering Corporation, Kenilworth, N.J.);
(c) Roferon®, recombinant interferon alfa-2a (Hoffmann-La Roche, Nutley, N.J.);
(d) Pegasys®, peginterferon alfa-2a (Hoffmann-La Roche, Nutley, N.J.);
(e) Berefor®, interferon alfa 2 available (Boehringer Ingelheim Pharmaceutical, Inc., Ridgefield, Conn.);
(f) Sumiferon®, a purified blend of natural alpha interferons (Sumitomo, Japan)
(g) Wellferon®, lymphoblastoid interferon alpha n1 (GlaxoSmithKline);
(h) Infergen®, consensus alpha interferon (InterMune Pharmaceuticals, Inc., Brisbane, Calif.);
(i) Alferon®, a mixture of natural alpha interferons (Interferon Sciences, and Purdue Frederick Co., Conn.);
(j) Viraferon®;
(k) Consensus alpha interferon from Amgen, Inc., Newbury Park, Calif.,

Other forms of interferon include: interferon beta, gamma, tau and omega, such as Rebif (Interferon beta 1a) by Serono, Omniferon (natural interferon) by Viragen, REBIF (interferon beta-1a) by Ares-Serono, Omega Interferon by BioMedicines; oral Interferon Alpha by Amarillo Biosciences; an interferon conjugated to a water soluble polymer or to a human albumin, e.g., Albuferon (Human Genome Sciences), an antiviral agent, a consensus interferon, ovine or bovine interferon-tau

Conjugates of interferon to a water-soluble polymer are meant to include especially conjugates to polyalkylene oxide homopolymers such as polyethylene glocol (PEG) or polypropylene glycols, polyoxyethylenated polyols, copolymers thereof and block copolymers thereof. As an alternative to polyalkylene oxid-based polymers, effectively non-antigenic materials such as dextran, polyvinyl pyrrolidones, polyacrylamides, polyvinyl alcohols, carbohydrate-based polymers and the like can be used. Since the polymeric modification sufficiently reduces antigenic response, the foreign interferon need not be completely autologous. Interferon used to prepare polymer conjugates may be prepared from a mammalian extract, such as human, ruminant or bovine interferon, or recombinantly produced. Preferred are conjugates of interferon to polyethylene glycol, also known as pegylated interferons.

(2) Ribavirin, such as ribavirin (1-beta-D-ribofuranosyl-1H-1,2,4-triazole-3-carboxamide) from Valeant Pharmaceuticals, Inc., Costa Mesa, Calif.); Rebetol® from Schering Corporation, Kenilworth, N.J., and Copegus® from Hoffmann-La Roche, Nutley, N.J.; and new ribavirin analogues in development such as Levovirin and Viramidine by Valeant,

(3) Thiazolidine derivatives which show relevant inhibition in a reverse-phase HPLC assay with an NS3/4A fusion protein and NS5A/5B substrate (Sudo K. et al., Antiviral Research, 1996, 32, 9-18), especially compound RD-1-6250, possessing a fused cinnamoyl moiety substituted with a long alkyl chain, RD4 6205 and RD4 6193;

(4) Thiazolidines and benzanilides identified in Kakiuchi N. et al. J. FEBS Letters 421, 217-220; Takeshita N. et al. Analytical Biochemistry, 1997, 247, 242-246;

(5) A phenan-threnequinone possessing activity against protease in a SDS-PAGE and autoradiography assay isolated from the fermentation culture broth of Streptomyces sp., Sch 68631 (Chu M. et al., Tetrahedron Letters, 1996, 37, 7229-7232), and Sch 351633, isolated from the fungus Penicillium griseofulvum, which demonstrates activity in a scintillation proximity assay (Chu M. et al, Bioorganic and Medicinal Chemistry Letters 9, 1949-1952);

(6) Protease inhibitors.

Examples include substrate-based NS3 protease inhibitors (Attwood et al., Antiviral peptide derivatives, PCT WO 98/22496, 1998; Attwood et al., Antiviral Chemistry and Chemotherapy 1999, 10, 259-273; Attwood et al, Preparation and use of amino acid derivatives as anti-viral agents, German Patent Pub. DE 19914474; Tung et al. Inhibitors of serine proteases, particularly hepatitis C virus NS3 protease; PCT WO 98/17679), including alphaketoamides and hydrazinoureas, and inhibitors that terminate in an electrophile such as a boronic acid or phosphonate (Llinas-Brunet et al. Hepatitis C inhibitor peptide analogues, PCT WO 99/07734) are being investigated. Non-substrate-based NS3 protease inhibitors such as 2,4,6-trihydroxy-3-nitro-benzamide derivatives (Sudo K. et al., Biochemiscal and Biophysical Research Communications, 1997, 238 643-647; Sudo K. et al. Antiviral Chemistry and Chemotherapy, 1998, 9, 186), including RD3-4082 and RD3-4078, the former substituted on the amide with a 14 carbon chain and the latter processing a para-phenoxyphenyl group are also being investigated.

Sch 68631, a phenanthrenequinone, is an HCV protease inhibitor (Chu M et al., Tetrahedron Letters 37:7229-7232, 1996). In another example by the same authors, Sch 351633, isolated from the fungus Penicillium grieofulvum, was identified as a protease inhibitor (Chu M. et al., Bioorganic and Medicinal Chemistry Letters 9:1949-1952). Nanomolar potency against the HCV NS3 protease enzyme has been achieved by the design of selective inhibitors based on the macromolecule eglin c. Eglin c, isolated from leech, is a potent inhibitor of several serine proteases such as S. griseus proteases A and B, ∀-chymotrypsin, chymase and subtilisin. Qasim M. A. et al., Biochemistry 36:1598-1607, 1997.

U.S. patents disclosing protease inhibitors for the treatment of HCV include, for example, U.S. Pat. No. 6,004,933 to Spruce et al (incorporated herein by reference in its entirety) which discloses a class of cysteine protease inhibitors for inhibiting HCV endopeptidase 2; U.S. Pat. No. 5,990,276 to Zhang et al.(incorporated herein by reference in its entirety) which discloses synthetic inhibitors of hepatitis C virus NS3 protease; U.S. Pat. No. 5,538,865 to Reyes et al. (incorporated herein by reference in its entirety). Peptides as NS3 serine protease inhibitors of HCV are disclosed in WO 02/008251 to Corvas International, Inc., and WO 02/08187 and WO 02/008256 to Schering Corporation (incorporated herein by reference in their entireties). HCV inhibitor tripeptides are disclosed in U.S. Pat. Nos. 6,534,523, 6,410,531 and 6,420,380 to Boehringer Ingelheim and WO 02/060926 to Bristol Myers Squibb (incorporated herein by reference in their entireties). Diaryl peptides as NS3 serine protease inhibitors of HCV are disclosed in WO 02/48172 to Schering Corporation (incorporated herein by reference). Imidazoleidinones as NS3 serine protease inhibitors of HCV are disclosed in WO 02/18198 to Schering Corporation and WO 02/48157 to Bristol Myers Squibb (incorporated herein by reference in their entireties). WO 98/17679 to Vertex Pharmaceuticals and WO 02/48116 to Bristol Myers Squibb also disclose HCV protease inhibitors (incorporated herein by reference in their entireties).

HCV NS3-4A serine protease inhibitors including BILN 2061 by Boehringer Ingelheim, VX-950 by Vertex, SCH 6/7 by Schering-Plough, and other compounds currently in preclinical development;

Substrate-based NS3 protease inhibitors, including alphaketoamides and hydrazinoureas, and inhibitors that terminate in an elecrophile such as a boronic acid or phosphonate; Non-substrate-based NS3 protease inhibitors such as 2,4,6-trihydroxy-3-nitro-benzamide derivatives including RD3-4082 and RD3-4078, the former substituted on the amide with a 14 carbon chain and the latter processing a para-phenoxyphenyl group; and Sch68631, a phenanthrenequinone, an HCV protease inhibitor.

Sch 351633, isolated from the fungus Penicillium griseofulvum was identified as a protease inhibitor. Eglin c, isolated from leech is a potent inhibitor of several serine proteases such as S. griseus proteases A and B, a-chymotrypsin, chymase and subtilisin.

U.S. Pat. No. 6,004,933 (incorporated herein by reference in its entirety) discloses a class of cysteine protease inhibitors from inhibiting HCV endopeptidase 2; synthetic inhibitors of HCV NS3 protease (pat), HCV inhibitor tripeptides (pat), diaryl peptides such as NS3 serine protease inhibitors of HCV (pat), Imidazolidindiones as NS3 serine protease inhibitors of HCV (pat).

Thiazolidines and benzanilides (ref). Thiazolidine derivatives which show relevant inhibition in a reverse-phase HPLC assay with an NS3/4A fusion protein and NS5A/5B substrate especially compound RD-16250 possessing a fused cinnamoyl moiety substituted with a long alkyl chain, RD4 6205 and RD4 6193

Phenan-threnequinone possessing activity against protease in a SDS-PAGE and autoradiography assay isolated from the fermentation culture broth of Streptomyces sp, Sch68631 and Sch351633, isolated from the fungus Penicillium griseofulvum, which demonstrates activity in a scintillation proximity assay.

(7) Nucleoside or non-nucleoside inhibitors of HCV NS5B RNA-dependent RNA polymerase, such as 2′-C-methyl-3′-O-L-valine ester ribofuranosyl cytidine (Idenix) as disclosed in WO 2004/002422 A2 (incorporated herein by reference in its entirety), R803 (Rigel), JTK-003 (Japan Tabacco), HCV-086 (ViroPharma/Wyeth) and other compounds currently in preclinical development;

gliotoxin (ref) and the natural product cerulenin;

2′-fluoronucleosides;

other nucleoside analogues as disclosed in WO 02/057287 A2, WO 02/057425 A2, WO 01/90121, WO 01/92282, and U.S. Pat. No. 6,812,219, the disclosures of which are incorporated herein by reference in their entirety.

Idenix Pharmaceuticals discloses the use of branched nucleosides in the treatment of flaviviruses (including HCV) and pestiviruses in International Publication Nos. WO 01/90121 and WO 01/92282 (incorporated herein by reference in their entireties). Specifically, a method for the treatment of hepatitis C infection (and flaviviruses and pestiviruses) in humans and other host animals is disclosed in the Idenix publications that includes administering an effective amount of a biologically active 1′, 2′, 3′ or 4′-branced B-D or B-L nucleosides or a pharmaceutically acceptable salt or prodrug thereof, administered either alone or in combination with another antiviral agent, optionally in a pharmaceutically acceptable carrier. Certain preferred biologically active 1′, 2′, 3′, or 4′ branched B-D or B-L nucleosides, including Telbivudine, are described in U.S. Pat. Nos. 6,395,716 and 6,875,751, each of which are incorporated herein by reference.

Other patent applications disclosing the use of certain nucleoside analogs to treat hepatitis C virus include: PCTCA00/01316 (WO 01/32153; filed Nov. 3, 2000) and PCT/CA01/00197 (WO 01/60315; filed Feb. 19, 2001) filed by BioChem Pharma, Inc., (now Shire Biochem, Inc.); PCT/US02/01531 (WO 02/057425; filed Jan. 18, 2002) and PCT/US02/03086 (WO 02/057287; filed Jan. 18, 2002) filed by Merck & Co., Inc., PCT/EP01/09633 (WO 02/18404; published Aug. 21, 2001) filed by Roche, and PCT Publication Nos. WO 01/79246 (filed Apr. 13, 2001), WO 02/32920 (filed Oct. 18, 2001) and WO 02/48165 by Pharmasset, Ltd. (the disclosures of which are incorporated herein by reference in their entireties)

PCT Publication No. WO 99/43691 to Emory University (incorporated herein by reference in its entirety), entitled “2′-Fluoronucleosides” discloses the use of certain 2′-fluoronucleosides to treat HCV.

Eldrup et al. (Oral Session V, Hepatitis C Virus, Flaviviridae; 16^{th} International Conference on Antiviral Research (Apr. 27, 2003, Savannah, Ga.)) described the structure activity relationship of 2′-modified nucleosides for inhibition of HCV.

Bhat et al. (Oral Session V, Hepatitis C Virus, Flaviviridae, 2003 (Oral Session V, Hepatitis C Virus, Flaviviridae; 16^{th} International conference on Antiviral Research (Apr. 27, 2003, Savannah, Ga.); p A75) describes the synthesis and pharmacokinetic properties of nucleoside analogues as possible inhibitors of HCV RNA replication. The authors report that 2′-modified nucleosides demonstrate potent inhibitory activity in cell-based replicon assays.

Olsen et al. (Oral Session V, Hepatitis C Virus, Flaviviridae; 16^{th} International Conference on Antiviral Research (Apr. 27, 2003, Savannah, Ga.) p A76) also described the effects of the 2′-modified nucleosides on HCV RNA replication.

(8) Nucleotide polymerase inhibitors and gliotoxin (Ferrari R. et al. Journal of Virology, 1999, 73, 1649-1654), and the natural product cerulenin (Lohmann V. et al. Virology, 1998, 249, 108-118);

(9) HCV NS3 helicase inhibitors, such as VP_{—}50406 by ViroPhama and compounds from Vertex. Other helicase inhibitors (Diana G. D. et al., Compounds, compositions and methods for treatment of hepatitis C, U.S. Pat. No. 5,633,358 (incorporated herein by reference in its entirety); Diana G. D. et al., Piperidine derivatives, pharmaceutical compositions thereof and their use in the treatment of hepatitis C, PCT WO 97/36554);

(10) Antisense phosphorothioate oligodeoxynucleotides (S-ODN) complementary to sequence stretches in the 5′ non-coding region (NCR) of the virus (Alt M. et al., Hepatology, 1995, 22, 707-717), or nucleotides 326-348 comprising the 3′ end of the NCR and nucleotides 371-388 located in the core coding region of the HCV RNA (Alt M. et al., Archives of Virology, 1997, 142, 589-599; Galderisi U. et al., Journal of Cellular Physiology, 199, 181, 251-257); such as ISIS 14803 by Isis Pharm/Elan, antisense by Hybridon, antisense by AVI bioPharma, (11) Inhibitors of IRES-dependent translation (Ikeda Net al., Agent for the prevention and treatment of hepatitis C, Japanese Patent Pub. JP-08268890; Kai Y et al. Prevention and treatment of viral diseases, Japanese Patent Pub. JP-10101591); such as ISIS 14803 by Isis Pharm/Elan, IRES inhibitor by Anadys, IRES inhibitors by Immusol, targeted RNA chemistry by PTC Therapeutics

(12) Ribozymes, such as nuclease-resistant ribozymes (Maccjak, D. J. et al., Hepatology 1999, 30, abstract 995) and those: directed in U.S. Pat. No. 6,043,077 to. Barber et al., and U.S. Pat. Nos. 5,869,253 and 5,610,054 to Draper et al. (incorporated herein by reference in their entireties) for example, HEPTAZYME by RPI

(13) siRNA directed against HCV genome

(14) HCV replication inhibitor of any other mechanisms such as by VP50406ViroPharama/Wyeth, inhibitors from Achillion, Arrow

(15) An inhibitor of other targets in the HCV life cycle including viral entry, assembly and maturation

(16) An immune modulating agent such as an IMPDH inhibitor, mycophenolic acid, a salt or a prodrug thereof sodium mycophenolate or mycophenolate mofetil, or Merimebodib (VX-497); thymosin alpha-1 (Zadaxin, by SciClone); or a S1P receptor agonist, e.g. FTY720 or analogue thereof optionally phosphorylated.

(17) An anti-fibrotic agent, such as a N-phenyl-2-pyrimidine-amine derivative, imatinib (Gleevac), IP-501 by Indevus, and Interferon gamma 1b from InterMune

(18) Therapeutic vaccine by Intercell, Epimmune/Genecor, Merix, Tripep (Chron-VacC), immunotherapy (Therapore) by Avant, T cell therapy by CellExSys, monoclonal antibody XTL-002 by STL, ANA 246 and ANA 246 BY Anadys,

(19) Other miscellaneous compounds including 1-amino-alkylcyclohexanes (U.S. Pat. No. 6,034,134 to Gold et al.), alkyl lipids (U.S. Pat. No. 5,922,757 to Chojkier et al.), vitamin E and other antitoxidants (U.S. Pat. No. 5,922,757 to Chojkier et al.), amantadine, bile acids (U.S. Pat. No. 5,846,99964 to Ozeki et al.), N-(phosphonoacetl)-L-aspartic acid,) U.S. Pat. No. 5,830,905 to Diana et al.), benzenedicarboxamides (U.S. Pat. No. 5,633,388 to Diane et al.), polyadenylic acid derivatives (U.S. Pat. No. 5,496,546 to Wang et al.), 2′3′-dideoxyinosine (U.S. Pat. No. 5,026,687 to Yarchoan et al.), benzimidazoles (U.S. Pat. No. 5,891,874 to Colacino et al.), plant extracts (U.S. Pat. No. 5,837,257 to Tsai et al., U.S. Pat. No. 5,725,859 to Omer et al., and U.S. Pat. No. 6,056,961) and piperidines (U.S. Pat. No. 5,830,905 to Diana et al.); the disclosures of which are incorporated herein by reference in their entireties. Also, squalene, telbivudine, N-(phosphonoacetyl)-L-aspartic acid, benzenedicarboxamides, polyadenylic acid derivatives, glycosylation inhibitors, and nonspecific cytoprotective agents that block cell injury caused by the virus infection.

(20) Any other compound currently in preclinical or clinical development for the treatment of HCV, including Interleukin-10 (Schering-Plough), AMANTADINE (Symmetrel) by Endo Labs Solvay, caspase inhibitor IDN-6556 by Idun Pharma, HCV/MF59 by Chiron, CIVACIR (Hepatitis C Immune Globulin) by NABI, CEPLENE (histamine dichloride) by Maxim, IDN-6556 by Idun PHARM, T67, a beta-tubulin inhibitor, by Tularik, a therapeutic vaccine directed to E2 by Innogenetics, FK788 by Fujisawa Helathcare, IdB1016 (Siliphos, oral silybin-phosphatidyl choline phytosome), fusion inhibitor by Trimeris, Dication by Immtech, hemopurifier by Aethlon Medical, UT 231B by United Therapeutics.

(21) Purine nucleoside analog antagonists of T1R7 (toll-like receptors) developed by Anadys, e.g., Isotorabine (ANA245) and its prodrug (ANA975), which are described in European applications EP348446 and EP636372, International Publications WO03/045968, WO05/121162 and WO05/25583, and U.S. Pat. No. 6,973,322, each of which is incorporated by reference.

(21) Non-nucleoside inhibitors developed by Genelabs and described in International Publications WO2004/108687, WO2005/12288, and WO2006/076529, each of which is incorporated by reference.

(22) Other co-agents (e.g., non-immunomodulatory or immunomodulatory compounds) that may be used in combination with a compound of this invention include, but are not limited to, those specified in WO 02/18369, which is incorporated herein by reference.

Methods of this invention may also involve administration of another component comprising an additional agent selected from an immunomodulatory agent; an antiviral agent; an inhibitor of HCV protease; an inhibitor of another target in the HCV life cycle; a CYP inhibitor; or combinations thereof.

Accordingly, in another embodiment, this invention provides a method comprising administering a compound of the invention and another anti-viral agent, preferably an anti-HCV agent. Such anti-viral agents include, but are not limited to, immunomodulatory agents, such as α, β, and δ interferons, pegylated derivatized interferon-a compounds, and thymosin; other anti-viral agents, such as ribavirin, amantadine, and telbivudine; other inhibitors of hepatitis C proteases (NS2-NS3 inhibitors and NS3-NS4A inhibitors); inhibitors of other targets in the HCV life cycle, including helicase, polymerase, and metalloprotease inhibitors; inhibitors of internal ribosome entry; broad-spectrum viral inhibitors, such as IMPDH inhibitors (e.g., compounds of U.S. Pat. Nos. 5,807,876, 6,498,178, 6,344,465, 6,054,472, WO 97/40028, WO 98/40381, WO 00/56331, and mycophenolic acid and derivatives thereof, and including, but not limited to VX-497, VX-148, and/or VX-944); or combinations of any of the above.

In accordance with the foregoing the present invention provides in a yet further aspect:
A pharmaceutical combination comprising a) a first agent which is a compound of the invention, e.g. a compound of formula I or any subformulae thereof, and b) a co-agent, e.g. a second drug agent as defined above.
A method as defined above comprising co-administration, e.g. concomitantly or in sequence, of a therapeutically effective amount of a compound of the invention, e.g. a compound of formula I or any subformulae thereof, and a co-agent, e.g. a second drug agent as defined above.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time. Fixed combinations are also within the scope of the present invention. The administration of a pharmaceutical combination of the invention results in a beneficial effect, e.g. a synergistic therapeutic effect, compared to a monotherapy applying only one of its pharmaceutically active ingredients.

Each component of a combination according to this invention may be administered separately, together, or in any combination thereof. As recognized by skilled practitioners, dosages of interferon are typically measured in IU (e.g., about 4 million IU to about 12 million IU).

If an additional agent is selected from another CYP inhibitor, the method would, therefore, employ two or more CYP inhibitors. Each component may be administered in one or more dosage forms. Each dosage form may be administered to the patient in any order. The compound of the invention and any additional agent may be formulated in separate dosage forms. Alternatively, to decrease the number of dosage forms administered to a patient, the compound of the invention and any additional agent may be formulated together in any combination. For example, the compound of the invention inhibitor may be formulated in one dosage form and the additional agent may be formulated together in another dosage form. Any separate dosage forms may be administered at the same time or different times.

Alternatively, a composition of this invention comprises an additional agent as described herein. Each component may be present in individual compositions, combination compositions, or in a single composition.

### EXEMPLIFICATION OF THE INVENTION

The invention is further illustrated by the following examples, which should not be construed as further limiting. The assays used throughout the Examples are accepted. Demonstration of efficacy in these assays is predictive of efficacy in subjects.

The following abbreviations are used throughout the examples and the specification.

### List of Abbreviations

abs. Absolute
Ac acetyl
ACN Acetonitrile
AcOEt/EtOAc Ethyl acetate
AcOH acetic acid
aq aqueous
Ar aryl
Bn benzyl
Bu butyl (nBu=n-butyl, tBu=tert-butyl)
CDI Carbonyldiimidazole
CH₃CN Acetonitrile
DBU 1,8-Diazabicyclo[5.4.0]-undec-7-ene
DCE 1,2-Dichloroethane
DCM Dichloromethane
DIPEA N-Ethyldiisopropylamine
DMAP Dimethylaminopyridine
DMF N,N′-Dimethylformamide
DMSO Dimethylsulfoxide
EI Electronspray ionisation
Et₂O Diethylether
Et₃N Triethylamine
Ether Diethylether
EtOH Ethanol
FC Flash Chromatography
h hour(s)
HATU O-(7-Azabenzotriazole-1-yl)-N,N,N′N′-tetramethyluronium hexafluorophosphate
HBTU O-(Benzotriazol-1-yl)-N,N,N′,N′-tetramethyluronium hexafluorophosphate hexafluorophosphate
HCl Hydrochloric acid
HOBt 1-Hydroxybenzotriazole
HPLC High Performance Liquid Chromatography
H₂O Water
L liter(s)
LC-MS Liquid Chromatography Mass Spectrometry
Me methyl
MeI Iodomethane
MeOH Methanol
mg milligram
min minute(s)
mL milliliter
MS Mass Spectrometry
Pd/C palladium on charcoal
PG protecting group
Ph phenyl
Prep Preparative
Rf ratio of fronts
RP reverse phase
rt Room temperature
SiO₂ Silica gel
TBAF Tetrabutylammonium fluoride
TEA Triethylamine
TFA Trifluoroacetic acid
THF Tetrahydrofurane
TLC Thin Layer Chromatography
t_{R} Retention time
Trademarks Hyflo=Celite® (The Celite Corporation)=filtering aid based on diatomaceous earth
Nucleosil=Nucleosil®, trademark of Machery & Nagel, Duren, FRG for HPLC materials
Temperatures are measured in degrees Celsius. Unless otherwise indicated, the reactions take place at roome temperature.
TLC conditions: Rf values for TLC are measured on 5×10 cm TLC plates, silica gel F254, Merck, Darmstadt, Germany.

### HPLC (Method A):

Instrument: Agilent system
column: Macherey-Nagel Nucleosil 100-3 C18 HD, particle size 3.5 □m, pore size 100 Å, length 70 mm, internal diameter 4 mm, flow 1.0 ml/min
solvent: CH₃CN (0.1% CF₃CO₂H); H₂O (0.1% CF₃CO₂H)
gradient: 0-6 min: 20-100% CH₃CN, 1.5 min: 100% CH₃CN, 0.5 min 100-20% CH₃CN

### HPLC (Method B):

Instrument: Kontron, Kroma-System
Column: Macherey-Nagel, Lichrosphere 100-5 RP 18
Solvent: CH₃CN (0.1% CF₃CO₂H); H₂O (0.1% CF₃CO₂H)
Gradient: 0-5 min: 10-100% CH₃CN; 5-7.5 min: 100% CH₃CN (Flow 1.5 mL/min)

### HPLC (Method C):

Instrument: Agilent system
column: waters symmetry C18, 3.5 microm, 2.1×50 mm, flow 0.6 ml/min
solvent: CH₃CN (0.1% CF₃CO₂H); H₂O (0.1% CF₃CO₂H)
gradient: 0-3.5 min: 20-95% CH₃CN, 3.5-5 min: 95% CH₃CN, 5.5-5.55 min 95% to 20% CH₃CN

### MS (Method D):

Instrument: Agilent 1100 Series
Detection: API-ES, positive/negative

### LC-MS (Method E):

Instrument: Agilent system
Column: Waters symmetry, 3.5 microm, 50×2.1 mm; 5 min, 20% to 95% CH₃CN
solvent: CH₃CN (0.1% HCO₂H); H₂O (0.1% HCO₂H)
gradient: 0-3.5 min: 20-95% CH₃CN, 3.5-5 min: 95% CH₃CN, 5.5-5.55 min 95% to 20% CH₃CN

### Preparative HPLC (Method F):

Instrument: Gilson system
column: waters C18 ODB, 5 microm, 50×19 mm
solvent: CH₃CN (0.1% HCO₂H); H₂O (0.1% HCO₂H)

### Preparative HPLC (Method G):

Instrument: Gilson
Column: Sun-Fire prep C18 OBD 5 microm, Column 19×50 mm (flow 20 mL/min) or
Column 30×100 mm (flow 40 mL/min)
Solvent: CH₃CN (0.1% CF₃CO₂H) and H₂O (0.1% CF₃CO₂H)
Gradient: 0-20 min: 5-100% CH₃CN

### General Synthetic Methods

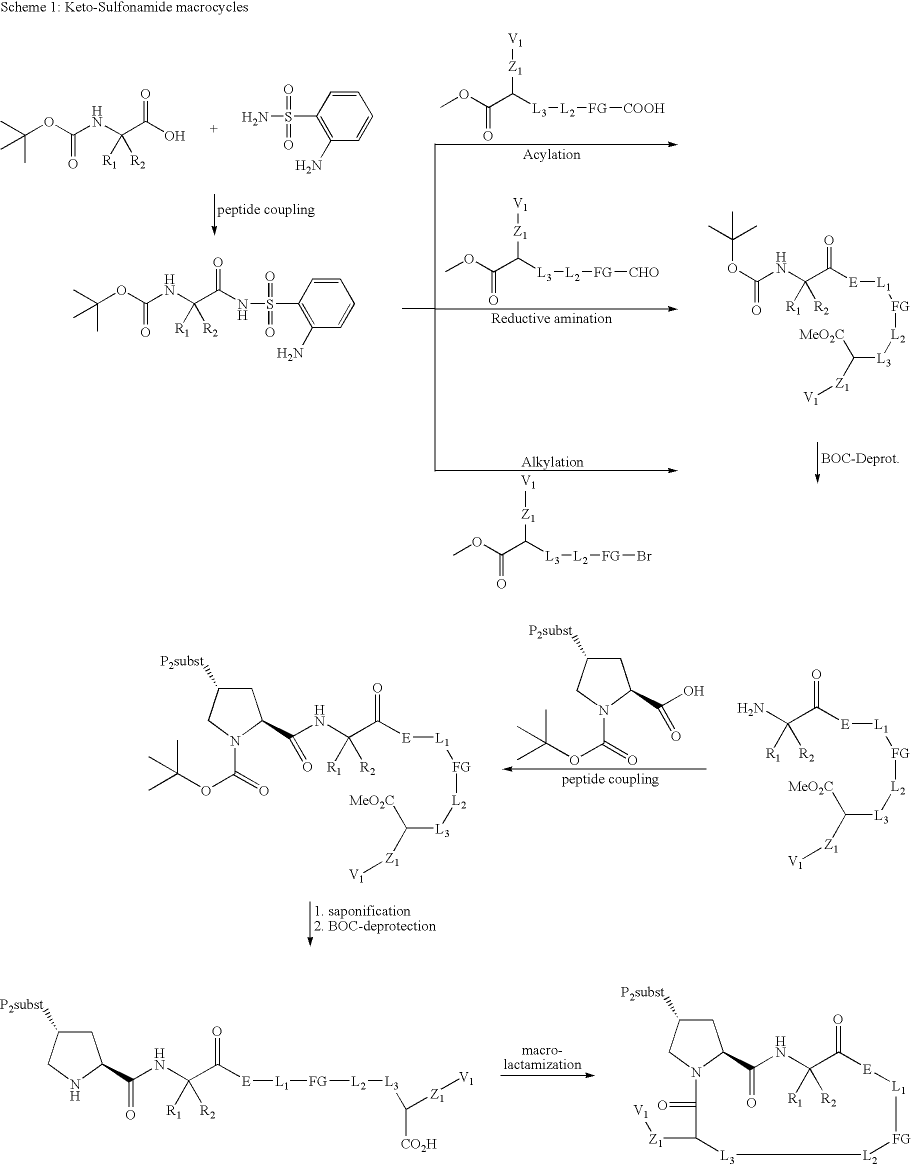

In Scheme 1, the term “linker” refers to the L₁-FG-L₂-L₃ residue of Formula I, the term “P₁” refers to the R₁ residue of Formula I, and the term “P₂subst” refers to the R₅ residue of Formula I.

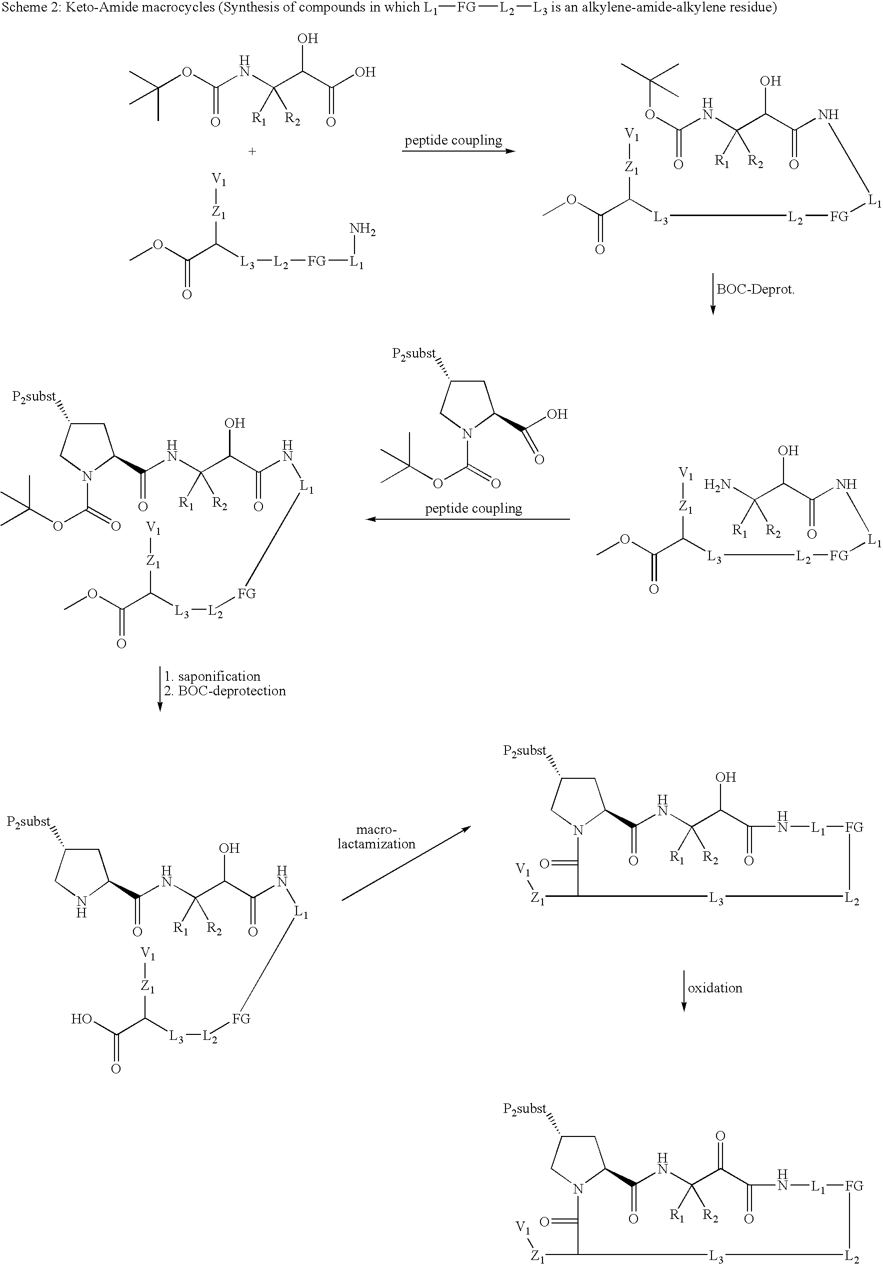

In Scheme 2, the term “linker” refers to the L₁-FG-L₂-L₃ residue of Formula I, the term “P₁” refers to the R₁ residue of Formula I, and the term “P₂subst” refers to the R₅ residue of Formula I.

### Example 1

### (8S,10R)-10-(7-Methoxy-2-phenyl-quinolin-4-yloxy)-5-[(1R,2S)-1-carbonylamino-2-vinyl-cyclopropyl]-2,2-dioxo-2λ*6*-thia-3,6,12,22-tetraaza-tricyclo[21.4.0.0*8,12*]heptacosa-1(27),23,25-triene-4,7,13,21-tetraone

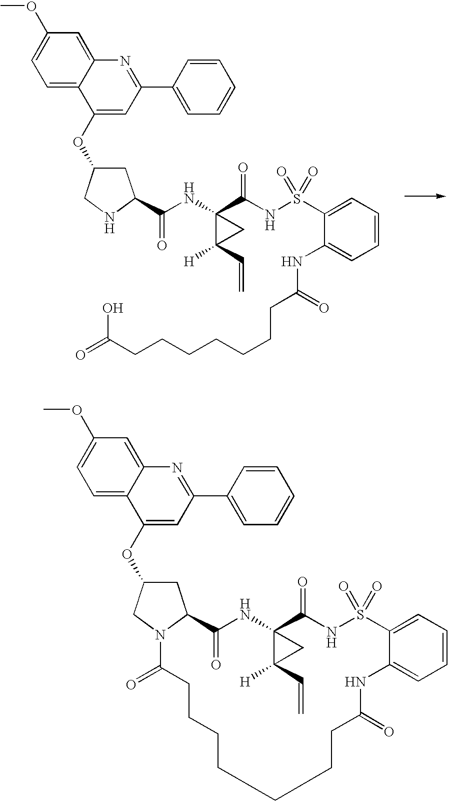

To an ice-cold solution of 250 mg (0.25 mmol) (8-{2-[((1R,2S)-1-{[(2S,4R)-4-(7-Methoxy-2-phenyl-quinolin-4-yloxy)-pyrrolidine-2-carbonyl]-amino}-2-vinyl-cyclopropanecarbonyl)-sulfamoyl]-phenyl-carbamoyl}-octanoic acid in 50 mL DCM/DMF (50:1) and 0.43 mL (2.5 mmol) of DIPEA is added 475 mg (1.3 mmol) HATU and the ice bath is removed. After stirring for 2 h the solvent is removed in vacuo and the residue is purified by preparative reverse phase HPLC (method G) to give the title compound as a colorless solid.

HPLC (method A) t_{R}=4.78 min

TLC, Rf (CH₂Cl₂/MeOH 9:1)=0.5

MS (method D): 780 [M+]

### Preparation of (8-{2-[((1R,2S)-1-{[(2S,4R)-4-(7-Methoxy-2-phenyl-quinolin-4-yloxy)-pyrrolidine-2-carbonyl]-amino}-2-vinyl-cyclopropanecarbonyl)-sulfamoyl]-phenylcarbamoyl}-octanoic acid

### Step 1

### [(1R,2S)-1-(2-Amino-benzenesulfonylaminocarbonyl)-2-vinyl-cyclopropyl]-carbamic acid tert-butyl ester

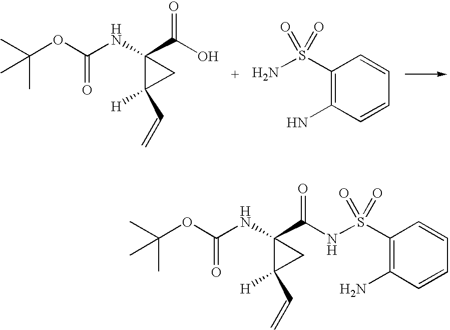

To a solution of 6.3 g (28 mmol) (1R,2S)-1-tert-Butoxycarbonylamino-2-vinyl-cyclopropane-carboxylic acid (prepared according to WO 2000009558 A1) in 90 mL abs. THF is added 6.95 g (42 mmol) CDI and the mixture is refluxed for 2 h. After cooling to rt 5.1 g (29 mmol) 2-Aminobenzenesulfonamide and 6.5 g (42 mmol) DBU is added and stirring is continued for 45 min. The reaction mixture is diluted with 250 mL EtOAc and washed with 100 mL 0.5 N HCl and brine. The organic phase is dried with Na₂SO₄, filtered and the solvent is removed in vacuo. The residue is purified by FC on silica (eluent: CH₂Cl₂/MeOH 98:2) to give the title compound as a colorless solid.

HPLC (method A) t_{R}=3.99 min

TLC, Rf (CH₂Cl₂/MeOH 19:1)=0.35

MS (method D): 382 [M+H]

### Step 2

### 8-{2-[((1R,2S)-1-tert-Butoxycarbonylamino-2-vinyl-cyclopropanecarbonyl)-sulfamoyl]-phenylcarbamoyl}-octanoic acid methyl ester

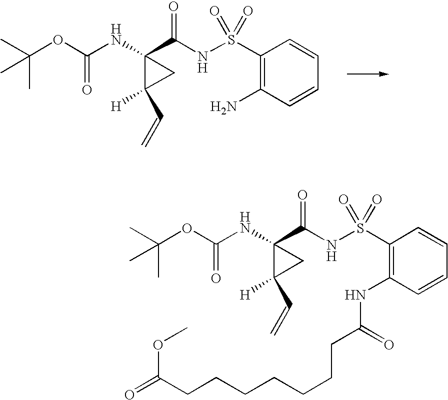

To a solution of 2.65 g (13 mmol) Monomethyl azelate in 20 mL DCM is added at rt a solution of 1.87 g (16 mmol) Benzotriazole and 1.87 g (16 mmol) Thionylchloride in 10 mL DCM. The suspension is stirred for 1 h, filtered, washed with 20 mL DCM and the solvent is removed in vacuo. The residue is dissolved in 10 mL DCM and added at 0° C. to a solution of 2.0 g (5.2 mmol) [(1R,2S)-1-(2-Amino-benzenesulfonylaminocarbonyl)-2-vinyl-cyclopropyl]-carbamic acid tert-butyl ester, 5.1 g (50 mmol) NEt₃ and 100 mg DMAP in 50 mL DCM. After stirring for 15 h at rt the reaction is quenched by addition of aq. bicarbonate, extracted with DCM, dried with Na₂SO₄, filtered and the solvent is removed in vacuo. The residue is purified by FC on silica (eluent: CH₂Cl₂/MeOH 98:2→95:5) to give the title compound as a red oil.

HPLC (method A) t_{R}=5.19 min

TLC, Rf (CH₂Cl₂/MeOH 9:1)=0.46

MS (method D): 566 [M+]

### Step 3

### 8-{2-[((1R,2S)-1-Amino-2-vinyl-cyclopropanecarbonyl)-sulfamoyl]-phenylcarbamoyl}-octanoic acid methyl ester

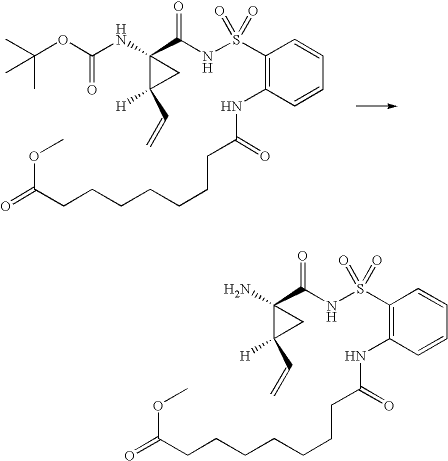

To a solution of 2.48 g (4.4 mmol) 8-{2-[((1R,2S)-1-tert-Butoxycarbonylamino-2-vinyl-cyclopropane-carbonyl)-sulfamoyl]-phenylcarbamoyl}-octanoic acid methyl ester in 4 mL Dioxane is added 6 mL 4N HCl in Dioxane at rt and the mixture is stirred for 15 h. The solvent is removed in vacuo to give the title compound as a hydrochloride salt which is used without further purification.

HPLC (method A) t_{R}=3.36 min

MS (method D): 466 [M+]

### Step 4

### (2S,4R)-2-{(1R,2S)-1-[2-(8-Methoxycarbonyl-octanoylamino)-benzenesulfonylaminocarbonyl]-2-vinyl-cyclopropylcarbamoyl}-4-(7-methoxy-2-phenyl-quinolin-4-yloxy)-pyrrolidine-1-carboxylic acid tert-butyl ester

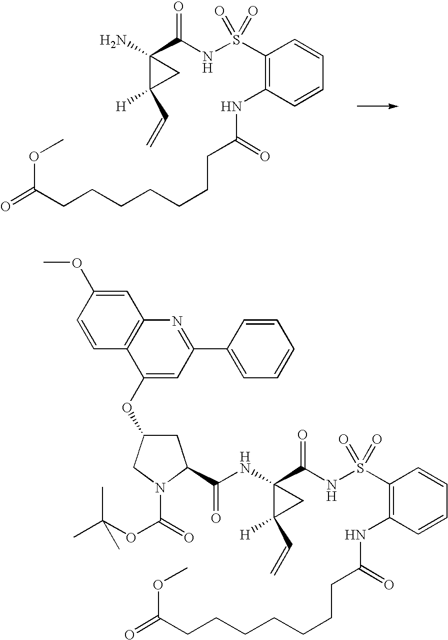

To an ice-cold solution of 0.39 g (0.78 mmol) 8-{2-[((1R,2S)-1-Amino-2-vinyl-cyclopropanecarbonyl)-sulfamoyl]-phenylcarbamoyl}-octanoic acid methyl ester (HCl-salt) in 25 mL DCM is added 0.44 g (0.94 mmol) (2S,4R)-4-(7-Methoxy-2-phenyl-quinolin-4-yloxy)-pyrrolidine-1,2-dicarboxylic acid 1-tert-butyl ester (prepared according to WO 2000009543), 0.46 g (1.2 mmol) HBTU and 0.51 g (3.9 mmol) DIPEA and the ice bath is removed. After stirring for 15 h at rt the reaction is quenched by addition of aq. bicarbonate, extracted with DCM, dried with Na₂SO₄, filtered and the solvent is removed in vacuo. The residue is purified by FC on silica (eluent: CH₂Cl₂/MeOH 99:1→95:5) to give the title compound as a colorless oil.

HPLC (method A) t_{R}=5.43 min

MS (method D): 912 [M+]

### Step 5

### 8-{2-[((1R,2S)-1-{[(2S,4R)-4-(7-Methoxy-2-phenyl-quinolin-4-yloxy)-pyrrolidine-2-carbonyl]-amino}-2-vinyl-cyclopropanecarbonyl)-sulfamoyl]-phenylcarbamoyl}-octanoic acid

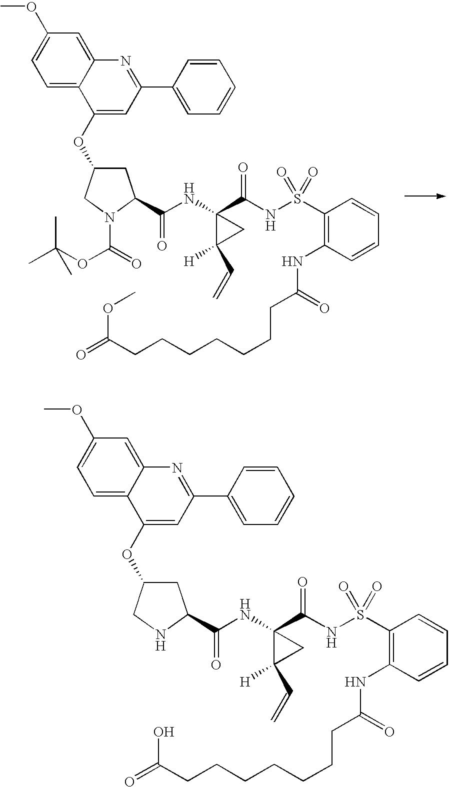

To a solution of 0.45 g (0.39 mmol) (2S,4R)-2-{(1R,2S)-142-(8-Methoxycarbonyl-octanoylamino)-benzenesulfonylaminocarbonyl]-2-vinyl-cyclopropylcarbamoyl}-4-(7-methoxy-2-phenyl-quinolin-4-yloxy)-pyrrolidine-1-carboxylic acid tert-butyl ester is added 2 mL TFA at rt. After stirring for 1 h the solvent is removed in vacuo, the residue is dissolved in 10 mL THF/MeOH/H₂O (2:1:1) and 50 mg (2.1 mmol) LiOH is added at rt. After stirring for 15 h, pH 5 is adjusted by addition of 1N HCl, the solvent is removed in vacuo, the residue is taken up in water and extracted with DCM. The combined organic phases are dried with Na₂SO₄, filtered and the solvent is removed in vacuo to give the title compound as a colorless oil, which is used without further purification.

HPLC (method A) t_{R}=4.49 min

MS (method D): 798 [M+]

### Example 2

### (8S,10R)-10-[2-(2-Isopropylamino-thiazol-4-yl)-7-methoxy-quinolin-4-yloxy]-5-[(1R,2)-1-carbonylamino-2-vinyl-cyclopropyl]-2,2-dioxo-2λ*6*-thia-3,6,12,22-tetraaza-tricyclo[21.4.0.0*8,12*]heptacosa-1(27),23,25-triene-4,7,13,21-tetraone

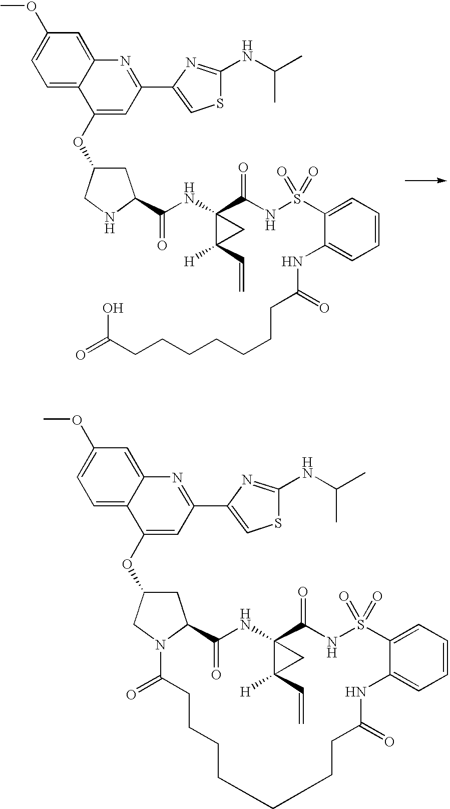

To a an ice-cold solution of 90 mg (0.10 mmol) 8-{2-[2-({(2S,4R)-4-[2-(2-Isopropylamino-thiazol-4-yl)-7-methoxy-quinolin-4-yloxy]-pyrrolidine-2-carbonyl}-amino)-2-methyl-propionylsulfamoyl]-phenylcarbamoyl}-octanoic acid in 26 mL DCM/DMF (25:1) is added 135 mg (1.04 mmol) DIPEA followed by 59 mg (0.16 mmol) HATU. After 15 min the ice bath is removed and stirring is continued at rt for 1 h. The solvent is removed in vacuo and the residue is purified by preparative reverse phase HPLC (Method G) to give the title compound as a yellow solid.

HPLC (method A) t_{R}=5.11 min

TLC, Rf (CH₂Cl₂/MeOH/H₂O/AcOH 75:27:5:0.5)=0.13

MS (method D): 844 [M+]

### Preparation of 8-{2-[2-({(2S,4R)-4-[2-(2-Isopropylamino-thiazol-4-yl)-7-methoxy-quinolin-4-yloxy]-pyrrolidine-2-carbonyl}-amino)-2-methyl-propionylsulfamoyl}-phenylcarbamoyl}-octanoic acid

### Step 1

### (2S,4R)-4-[2-(2-Isopropylamino-thiazol-4-yl)-7-methoxy-quinolin-4-yloxy]-2-{(1R,2S)-1-[2-(8-methoxycarbonyl-octanoylamino)-benzenesulfonylaminocarbonyl]-2-vinyl-cyclopropylcarbamoyl}-pyrrolidine-1-carboxylic acid tert-butyl ester

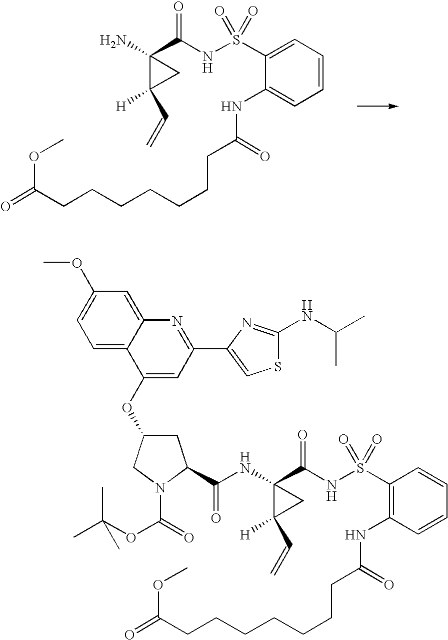

The title compound is prepared analogously as described for the title compound in Example 1 (step 4) using 91 mg (0.18 mmol) 8-{2-[((1R,2S)-1-Amino-2-vinyl-cyclopropanecarbonyl)-sulfamoyl]-phenyl-carbamoyl}-octanoic acid methyl ester (HCl-salt), 95 mg (0.18 mmol) (2S,4R)-4-[2-(2-Isopropylamino-thiazol-4-yl)-7-methoxy-quinolin-4-yloxy]-pyrrolidine-1,2-dicarboxylic acid 1-tert-butyl ester (prepared according to WO 2005073216), 89 mg (0.23 mmol) HATU and 116 mg (0.90 mmol) DIPEA in 5 mL DMF.

HPLC (method A) t_{R}=5.71 min

TLC, Rf (CH₂Cl₂/MeOH 9:1)=0.42

MS (method D): 976 [M+]

### Step 2

### (2S,4R)-2-{(1R,2S)-1-[2-(8-Carboxy-octanoylamino)-benzenesulfonylaminocarbonyl]-2-vinyl-cyclopropylcarbamoyl}-4-[2-(2-isopropylamino-thiazol-4-yl)-7-methoxy-quinolin-4-yloxy{-pyrrolidine-1-carboxylic acid tert-butyl ester

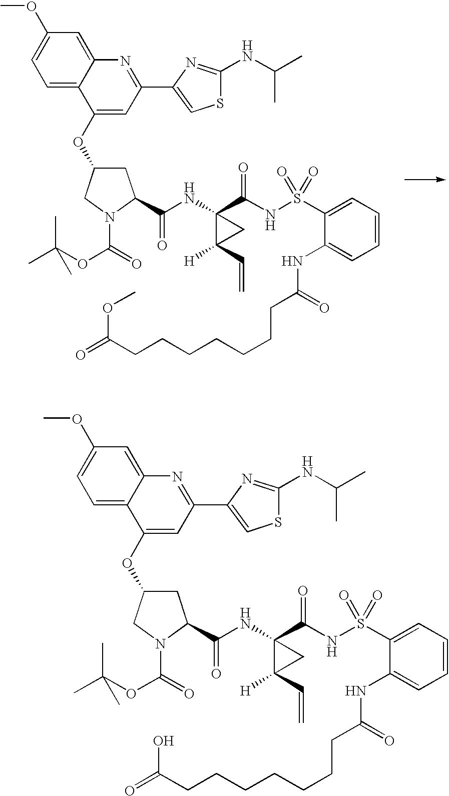

To a solution of 103 mg (0.10 mmol) (2S,4R)-4-[2-(2-Isopropylamino-thiazol-4-yl)-7-methoxy-quinolin-4-yloxy]-2-{(1R,2S)-1-[2-(8-methoxycarbonyl-octanoylamino)-benzenesulfonylaminocarbonyl]-2-vinyl-cyclopropylcarbamoyl}-pyrrolidine-1-carboxylic acid tert-butyl ester in 8 mL THF/MeOH/H₂O (2:1:1) is added 26 mg (1.1 mmol) LiOH at rt and the mixture is stirred for 2 h at 40° C. The solvent is removed in vacuo, pH 3 is adjusted by addition of 1N HCl followed by extraction with DCM. The combined organic phase is washed with brine, dried with Na₂SO₄, filtered and the solvent is removed in vacuo to give the title compound as a yellow oil, which is used without further purification.

HPLC (method A) t_{R}=5.23 min

TLC, Rf (CH₂Cl₂/MeOH 9:1)=0.20

MS (method D): 962 [M+]

### Step 3

### 8-(2-{[(1R,2S)-1-({(2S,4R)-4-[2-(2-Isopropylamino-thiazol-4-yl)-7-methoxy-quinoloin-4-yloxy]-pyrrolidine-2-carbonyl}-amino)-2-vinyl-cyclopropanecarbonyl]-sulfamoyl}-phenylcarbamoyl)-octanoic acid

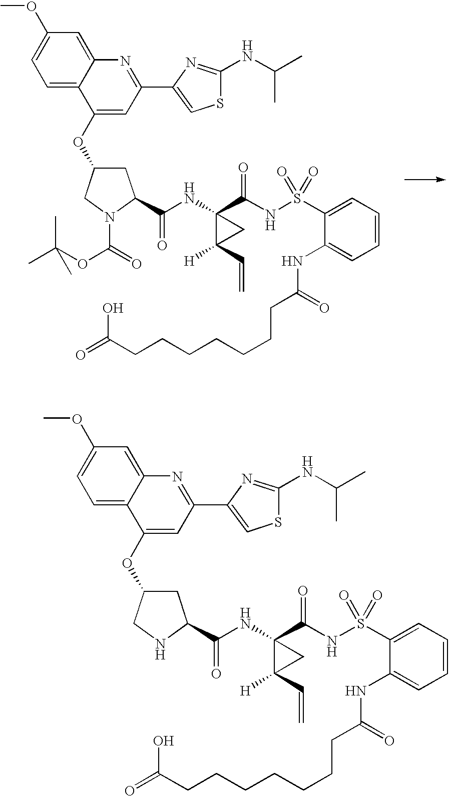

To a solution of 102 mg (0.11 mmol) (2S,4R)-2-{(1R,2S)-1-[2-(8-Carboxy-octanoylamino)-benzenesulfonylaminocarbonyl]-2-vinyl-cyclopropylcarbamoyl}-4-[2-(2-isopropylamino-thiazol-4-yl)-7-methoxy-quinolin-4-yloxy]-pyrrolidine-1-carboxylic acid tert-butyl ester in 5 mL DCM is added 0.5 mL TFA at rt. After stirring for 2 h the solvent is removed in vacuo. To remove excess of TFA the residue is taken up in DCM and the solvent is removed in vacuo. This procedure is repeated three times. The title compound is obtained as a brown oil, which is used without further purification.

HPLC (method A) t_{R}=4.55 min

TLC, Rf (CH₂Cl₂/MeOH/H₂O/AcOH 90:10:1:0.5)=0.49

MS (method D): 862 [M+]

### Example 3

### 4-Fluoro-1,3-dihydro-isoindole-2-carboxylic acid (8S,10R)-5-[(1R,2S)-1-carbonylamino-2-vinyl-cyclopropyl]-2,2,4,7,13,21-hexaoxo-2λ*6*-thia-3,6,12,22-tetraaza-tricyclo[21.4.0.0*8,12*]heptacosa-1(27),23,25-trien-10-yl ester

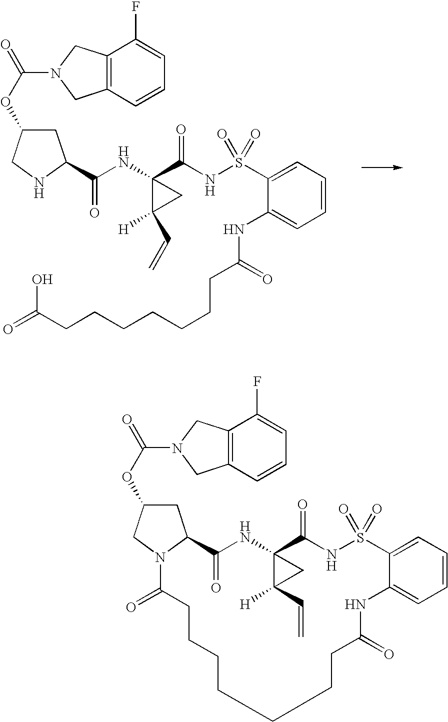

The title compound is prepared analogously as described for the title compound in Example 2 using 119 mg (0.14 mmol) 4-Fluoro-1,3-dihydro-isoindole-2-carboxylic acid (3R,5S)-5-{(1R,2S)-1-[2-(8-carboxy-octanoylamino)-benzenesulfonylaminocarbonyl]-2-vinyl-cyclopropylcarbamoyl}-pyrrolidin-3-yl ester (TFA-salt), 182 mg (1.4 mmol) DIPEA and 268 mg (0.71 mmol) HATU.

HPLC (method A) t_{R}=5.00 min

TLC, Rf (CH₂Cl₂/MeOH 19:1)=0.41

MS (method D): 710 [M+]+727 [M+H₂O]

### Preparation of (2S,4R)-4-(4-Fluoro-1,3-dihydro-isoindole-2-carbonyloxy)-pyrrolidine-1,2-dicarboxylic acid 1-tert-butyl ester

### Step 1

### (2S,4R)-4-(4-Fluoro-1,3-dihydro-isoindole-2-carbonyloxy)-pyrrolidine-1,2-dicarboxylic acid 1-tert-butyl ester 2-methyl ester

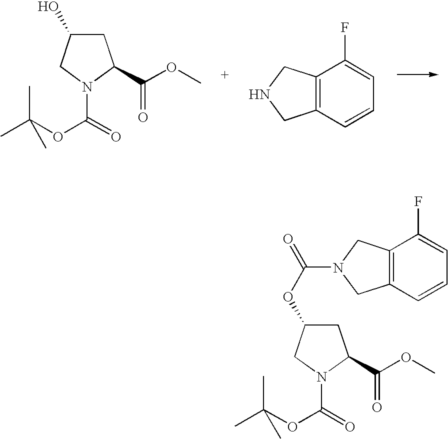

To a solution of 1.79 g (7.1 mmol) 2S,4R)-4-Hydroxy-pyrrolidine-1,2-dicarboxylic acid 1-tert-butyl ester 2-methyl ester in 65 mL DCM is added 1.57 (9.2 mmol) CDI at rt and the mixture is stirred for 1 h. A solution of 2.91 g (21.2. mmol) 4-Fluoro-2,3-dihydro-1H-isoindole (prepared according to WO 2005037214) in 5 mL DCM is added and the reaction mixture is stirred at rt overnight. The mixture is diluted with DCM and washed three times with 1N HCl, sat. NaHCO₃ and brine. The organic phase is dried with Na₂SO₄, filtered and the solvent is removed in vacuo. The residue is purified by FC (CH₂Cl₂/MeOH 98:2) to give the title compound as an oil.

LC-MS (method E) t_{R}=3.76 min, [M-BOC]=308

TLC, Rf (CH₂Cl₂/MeOH 9:1)=0.85

### Step 2

### (2S,4R)-4-(4-Fluoro-1,3-dihydro-isoindole-2-carbonyloxy)-pyrrolidine-1,2-dicarboxylic acid 1-tert-butyl ester

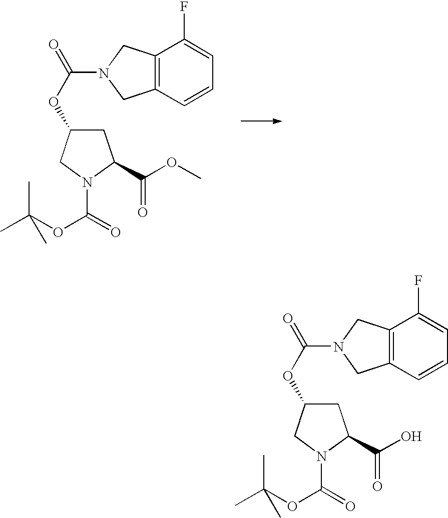

To a mixture of 500 mg (1.2 mmol) (2S,4R)-4-(4-Fluoro-1,3-dihydro-isoindole-2-carbonyloxy)-pyrrolidine-1,2-dicarboxylic acid 1-tert-butyl ester 2-methyl ester in 10 mL THF/methanol/water (3:1:1) is added 62 mg (1.5 mmol) lithiumhydroxid-hydrate and the mixture is stirred at rt for 6 h. pH is adjusted to 3 and the mixture is extracted four times with DCM. The combined organic layers are washed with NaHCO₃ and brine, dried over Na₂SO₄, filtered and concentrated in vacuo to yield the title compound which was used without further purification.

HPLC (method B) t_{R}=3.15 min

LC-MS (method E) t_{R}=3.49 min, [M−H]=394

TLC, Rf (CH₂Cl₂/MeOH 9:1)=0.48

### Preparation of 4-Fluoro-1,3-dihydro-isoindole-2-carboxylic acid (3R,5S)-1-tert-butoxycarbonyl-5-{(1R,2S)-1-[2-(8-carboxy-octanoylamino)-benzenesulfonylaminocarbonyl]-2-vinyl-cyclopropylcarbamoyl}-pyrrolidin-3-yl ester

### Step 1

### 4-Fluoro-1,3-dihydro-isoindole-2-carboxylic acid (3R,5S)-1-tert-butoxycarbonyl-5-{(1R,2S)-1-[2-(8-methoxycarbonyl-octanoylamino)-benzenesulfonylaminocarbonyl]-2-vinyl-cyclopropylcarbamoyl}-pyrrolidin-3-yl ester

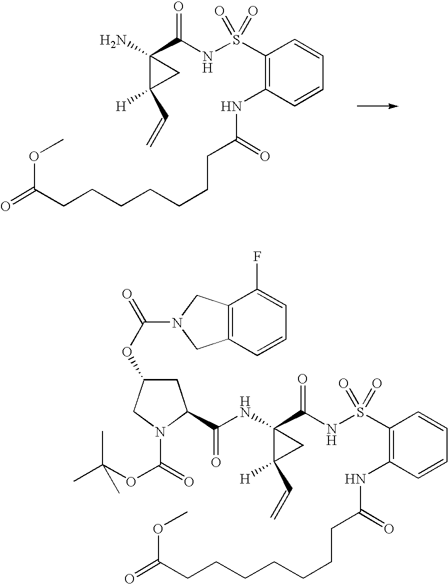

The title compound is prepared analogously as described for the title compound in Example 1 (step 4) using 200 mg (0.14 mmol) 8-{2-[((1R,2S)-1-Amino-2-vinyl-cyclopropanecarbonyl)-sulfamoyl]-phenyl carbamoyl}-octanoic acid methyl ester (HCl-salt), 113 mg (0.29 mmol) (2S,4R)-4-(4-Fluoro-1,3-dihydro-isoindole-2-carbonyloxy)-pyrrolidine-1,2-dicarboxylic acid 1-tert-butyl ester, 136 mg (0.36 mmol) HATU and 93 mg (0.71 mmol) DIPEA in 5 mL DCM.

HPLC (method A)=5.72 min

TLC, Rf (CH₂Cl₂/MeOH 9:1)=0.50

MS (method D): 859 [M+H₂O]

### Step 2

### 4-Fluoro-1,3-dihydro-isoindole-2-carboxylic acid (3R,5S)-1-tert-butoxycarbonyl-5-{(1R,2S)-1-[2-(8-carboxy-octanoylamino)-benzenesulfonylaminocarbonyl]-2-vinyl-cyclopropylcarbamoyl}-pyrrolidin-3-yl ester

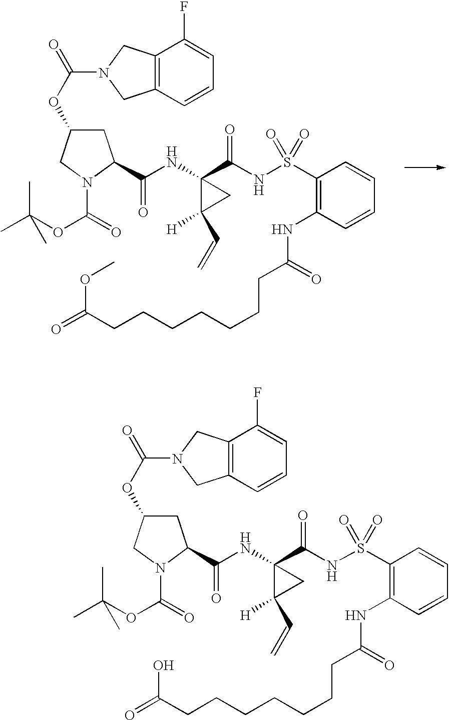

To a solution of 118 mg (0.14 mmol) 4-Fluoro-1,3-dihydro-isoindole-2-carboxylic acid (3R,5S)-1-tert-butoxycarbonyl-5- ((1R,2S)-1-[2-(8-methoxycarbonyl-octanoylamino)-benzensulfonylaminocarbonyl]-2-vinyl-cyclopropylcarbamoyl}-pyrrolidin-3-yl ester in 16 mL THF/MeOH/H₂O (2:1:1) is added 34 mg (1.4 mmol) LiOH at rt and the mixture is stirred for 2 h at 40° C. The solvent is removed in vacuo, pH 3 is adjusted by addition of 1N HCl followed by extraction with DCM. The combined organic phase is washed with brine, dried with Na₂SO₄, filtered and the solvent is removed in vacuo to give the title compound as a yellow oil, which is used without further purification.

t_{R} HPLC (method A) t_{R}=5.17 min

TLC, Rf (CH₂Cl₂/MeOH 85:15)=0.54

MS (method D): 845 [M+H₂O]

### Step 3

### 4-Fluoro-1,3-dihydro-isoindole-2-carboxylic acid (3R,5S)-1-tert-butoxycarbonyl-5-{(1R,2S)-1-[2-(8-carboxy-octanoylamino)-benzenesulfonylaminocarbonyl]-2-vinyl-cyclopropylcarbamoyl}-pyrrolidin-3-yl ester

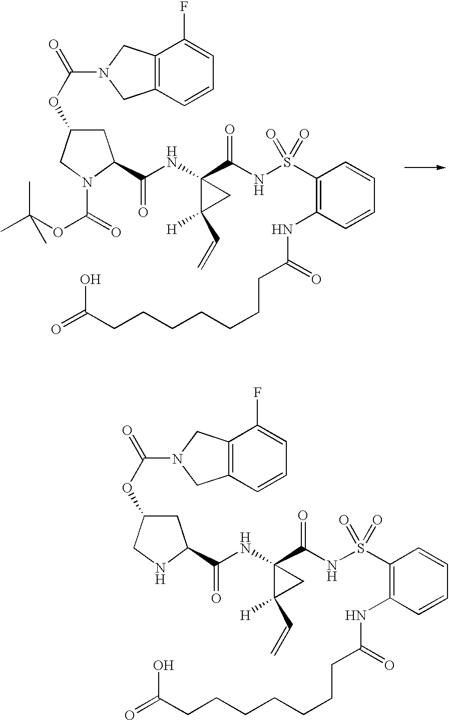

To a solution of 116 mg (0.14 mmol) 4-Fluoro-1,3-dihydro-isoindole-2-carboxylic acid (3R,5S)-1-tert-butoxycarbonyl-5- ((1R,2S)-1-[2-(8-carboxy-octanoylamino)-benzenesulfonylaminocarbonyl]-2-vinyl-cyclopropylcarbamoyl}-pyrrolidin-3-yl ester in 25 mL DCM is added 1 mL TFA at rt. After stirring overnight the solvent is removed in vacuo. To remove excess of TFA the residue is taken up in DCM and the solvent is removed in vacuo, which is repeated three times. The title compound is obtained as a brown oil, which is used without further purification.

HPLC (method A) t_{R}=4.22 min

TLC, Rf (CH₂Cl₂/MeOH 85:15)=0.56

MS (method D): 728 [M+]

### Example 4

### 11-[2-(1,2,3,4-tetrahydronaphthalene)]-8-[(1R,2S)-1-carbonylamino-2-vinyl-cyclopropyl]-5,5-dioxo-5,8,9,11,12,15,16,17,18,19,20,22-dodecahydro-6H,14H-5λ*6*-thia-6,9,12,22-tetraaza-benzocycloicosene-7,10,13,21-tetraone

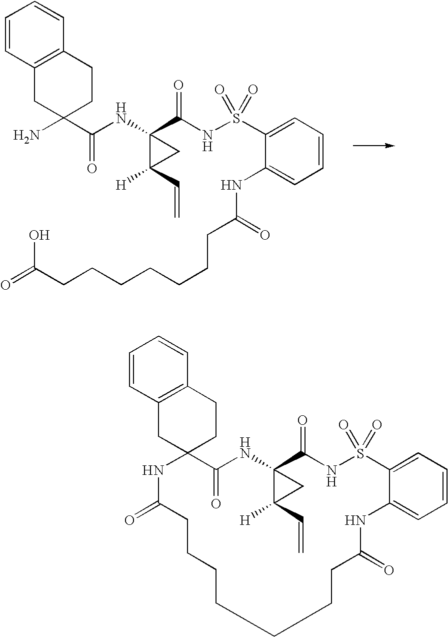

The title compound is prepared analogously as described for the title compound in Example 2 using 65 mg (0.09 mmol) 8-[2-({(1R,2S)-1-[(2-Amino-1,2,3,4-tetrahydro-naphthalene-2-carbonyl)-amino]-2-vinyl-cyclopropanecarbonyl}-sulfamoyl)-phenylcarbamoyl]-octanoic acid (TFA-salt), 114 mg (0.88 mmol) DIPEA and 167 g (0.44 mmol) HATU.

HPLC (method A)=5.07 min

TLC, Rf (CH₂Cl₂/MeOH 85:15)=0.23

MS (method D): 607 [M+]

### Preparation of 8-[2-({(1R,2S)-1-[(2-Amino-1,2,3,4-tetrahydro-naphthalene-2-carbonyl)-amino]-2-vinyl-cyclopropanecarbonyl}-sulfamoyl)-phenylcarbamoyl]-octanoic acid

### Step 1

### 8-[2-({(1R,2S)-1-[(2-tert-Butoxycarbonylamino-1,2,3,4-tetrahydro-naphthalene-2-carbonyl)-amino]-2-vinyl-cyclopropanecarbonyl}-sulfamoyl)-phenylcarbamoyl]-octanoic acid methyl ester

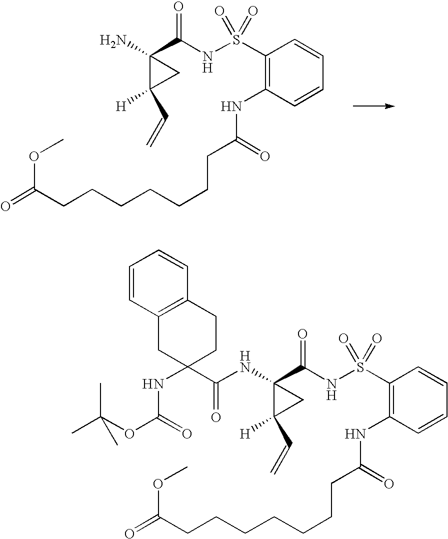

The title compound is prepared analogously as described for the title compound in Example 1 (step 4) using 150 mg (0.19 mmol) 8- {2-[((1R,2S)-1-Amino-2-vinyl-cyclopropanecarbonyl)-sulfamoyl]-phenylcarbamoyl)-octanoic acid methyl ester (HCl-salt), 66 mg (0.22 mmol) 2-tert-Butoxycarbonylamino-1,2,3,4-tetrahydro-naphthalene-2-carboxylic acid, 84 mg (0.22 mmol) HBTU and 120 mg (0.93 mmol) DIPEA in 2 mL DMF.

HPLC (method A) t_{R}=5.77 min

TLC, Rf (CH₂Cl₂/MeOH 19:1)=0.53

MS (method D): 739 [M+]

### Step 2

### 8-[2-({(1R,2S)-1-[(2-Amino-1,2,3,4-tetrahydro-naphthalene-2-carbonyl)-amino]-2-vinyl-cyclopropanecarbonyl}-sulfamoyl)-phenylcarbamoyl]-octanoic acid

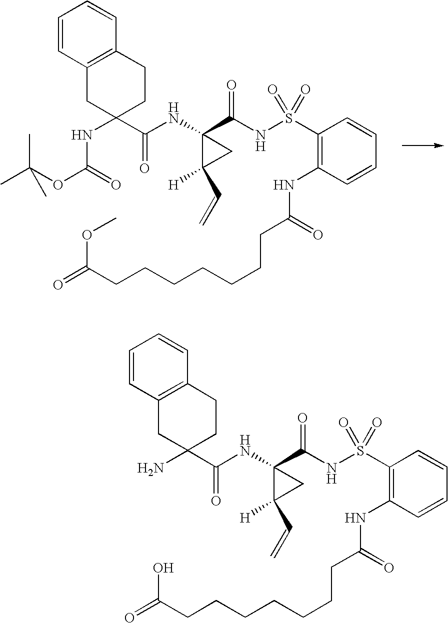

The title compound is prepared analogously as described for the title compound in Example 1 (step 5) using 102 mg (0.14 mmol) 8-[2-({(1R,2S)-1-[(2-tert-Butoxycarbonylamino-1,2,3,4-tetrahydro-naphthalene-2-carbonyl)-amino]-2-vinyl-cyclopropanecarbonyl}-sulfamoyl)-phenylcarbamoyl]-octanoic acid methyl ester and 1 mL TFA in 10 mL DCM and 33 mg (1.4 mmol) LiOH in 12 mL THF/MeOH/H₂O (2:1:1).

HPLC (method A) t_{R}=3.93 min

TLC, Rf (CH₂Cl₂/MeOH 19:1)=0.44

MS (method D): 625 [M+]

### Example 5

### 11-[2-indanyl]-8-[(1R,2S)-1-carbonylamino-2-vinyl-cyclopropyl]-5,5-dioxo-5,8,9,11,12,15,16,17,18,19,20,22-dodecahydro-6H,14H-5λ*6*-thia-6,9,12,22-tetraaza-benzocycloicosene-7,10,13,21-tetraone

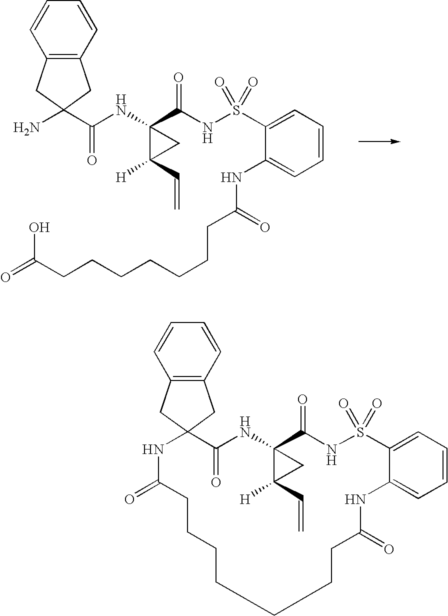

The title compound is prepared analogously as described for the title compound in Example 2 using 83 mg (0.012 mmol) 8-[2-({(1R,2S)-1-[(2-Amino-indane-2-carbonyl)-amino]-2-vinyl-cyclopropane-carbonyl}-sulfamoyl)-phenylcarbamoyl]-octanoic acid (TFA-salt), 149 mg (1.15 mmol) DIPEA and 219 g (0.58 mmol) HATU.

HPLC (method A) t_{R}=4.91 min

TLC, Rf (CH₂Cl₂/MeOH 9:1)=0.25

MS (method D): 593 [M+]

### Preparation of 8-[2-({(1R,2S)-1-[(2-Amino-indane-2-carbonyl)-amino]-2-vinyl-cyclopropane-carbonyl}-sulfamoyl)-phenylcarbamoylpoctanoic acid

### Step 1

### 8-[2-({(1R,2S)-1-[(2-tert-Butoxycarbonylamino-indane-2-carbonyl)-amino]-2-vinyl-cyclopropanecarbonyl}-sulfamoyl)-phenylcarbamoyl]-octanoic acid methyl ester

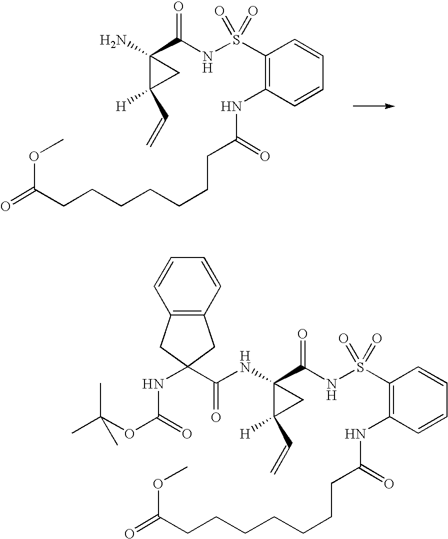

The title compound is prepared analogously as described for the title compound in Example 1 (step 4) using 163 mg (0.20 mmol) 8-{2-[((1R,2S)-1-Amino-2-vinyl-cyclopropanecarbonyl)-sulfamoyl]-phenylcarbamoyl}-octanoic acid methyl ester (HCl-salt), 67 mg (0.24 mmol) 2-tert-Butoxycarbonylamino-indan-2-carboxylic acid, 91 mg (0.24 mmol) HBTU and 130 mg (1.00 mmol) DIPEA in 2 mL DMF.

HPLC (method A) t_{R}=5.61 min

TLC, Rf (CH₂Cl₂/MeOH 19:1)=0.41

MS (method D): 725 [M+]

### Step 2

### 8-[2-({(1R,2S)-1-[(2-tert-Butoxycarbonylamino-indane-2-carbonyl)-amino]-2-vinyl-cyclopropanecarbonyl}-sulfamoyl)-phenylcarbamoyl]-octanoic acid

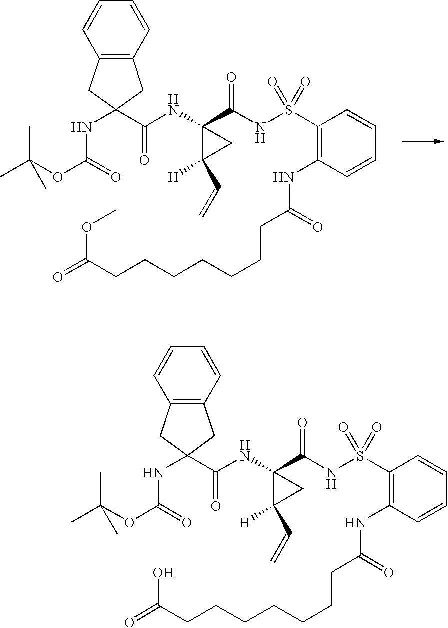

The title compound is prepared analogously as described for the title compound in Example 2 (step 2) using 84 mg (0.12 mmol) 8-[2-({(1R,2S)-1-[(2-tert-Butoxycarbonylamino-indane-2-carbonyl)-amino]-2-vinyl-cyclopropanecarbonyl}-sulfamoyl)-phenylcarbamoy]-octanoic acid methyl ester and 28 mg (1.16 mmol) LiOH in 10 mL THF/MeOH/H₂O (2:1:1).

HPLC (method A) t_{R}=5.02 min

TLC, Rf (CH₂Cl₂/MeOH 9:1)=0.35

MS (method D): 711 [M+]

### Step 3

### 8-[2-({(1R,2S)-1-[(2-tert-Butoxycarbonylamino-indane-2-carbonyl)-amino]-2-vinyl-cyclopropanecarbonyl}-sulfamoyl)-phenylcarbamoyl]-octanoic acid

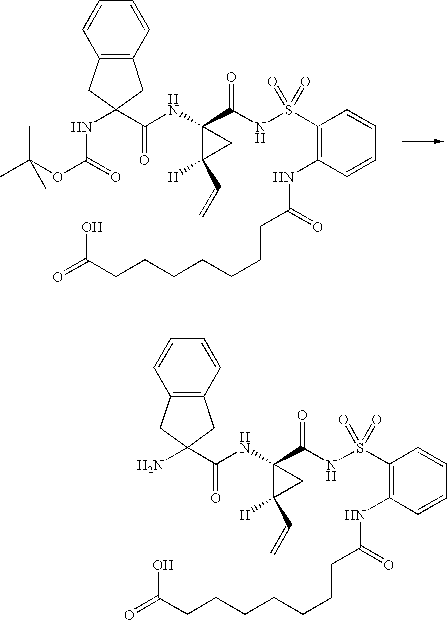

The title compound is prepared analogously as described for the title compound in Example 2 (step 3) using 82 mg (0.12 mmol) 8-[2-({(1R,2S)-1-[(2-tert-Butoxycarbonylamino-indane-2-carbonyl)-amino]-2-vinyl-cyclopropanecarbonyl}-sulfamoyl)-phenylcarbamoyl]-octanoic acid and 1 mL TFA in 25 mL DCM.

HPLC (method A) t_{R}=2.85 min

MS (method D): 611 [M+]

### Example 6

### 12-Cyclopentylmethyl-8-[(1R,2S)-1-carbonylamino-2-vinyl-cyclopropyl]-5,5-dioxo-5,8,9,11,12,15,16,17,18,19,20,22-dodecahydro-6H,14H-5λ*6*-thia-6,9,12,22-tetraaza-benzocycloicosene-7,10,13,21-tetraone

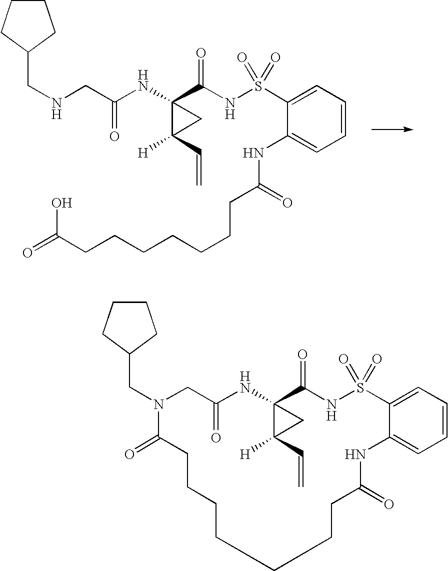

The title compound is prepared analogously as described for the title compound in Example 2 using 58 mg (0.08 mmol) 8-[2-({(1R,2S)-142-(Cyclopentylmethyl-amino)-acetylamino]-2-vinyl-cyclopropane-carbonyl}-sulfamoyl)-phenylcarbamoyl]-octanoic acid (TFA-salt), 106 mg (0.82 mmol) D1PEA and 156 mg (0.41 mmol) HATU in 51 mL DCM/MeOH (50:1).

HPLC (method A) t_{R}=5.23 min

TLC, Rf (CH₂Cl₂/MeOH 85:15)=0.23

MS (method D): 573 [M+H] +590 [M+H₂O]

### Preparation of (tert-Butoxycarbonyl-cyclopentylmethyl-amino)-acetic acid

### Step 1

### (Cyclopentylmethyl-amino)-acetic acid methyl ester

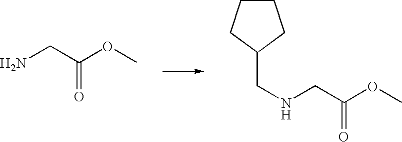

To a solution of 9.0 g (89 mmol) Cyclopentanecarboxaldehyde, 11.3 g (89 mmol) Glycine methylester hydrochloride and 13.1 g (116 mmol) NEt₃ in 250 mL MeOH is added 2 g molecular sieves 4A. After stirring for 30 min at rt, 4.5 g (116 mmol) NaBH₄ is added at 0° C. in 5 portions. The ice-bath is removed and stirring is continued for 2 h at rt. The reaction is quenched by addition of aq. bicarbonate, MeOH is evaporated and the residue is diluted with water. After extraction with DCM, the organic phase is washed with brine, dried with Na₂SO₄, filtered and the solvent is removed in vacuo. The residue is purified by FC on silica (eluent: hexane/EtOAc 3:1) to give the title compound as a yellow oil.

TLC, Rf (hexane/EtOAc 1:1)=0.55

MS (method D): 172 [M+H]

### Step 2

### (tert-Butoxycarbonyl-cyclopentylmethyl-amino)-acetic acid methyl ester

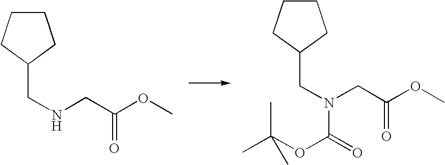

A solution of 1.1 g (6.2 mmol) (Cyclopentylmethyl-amino)-acetic acid methyl ester and 1.25 g (12.4 mmol) NEt₃ in 60 mL DCM is cooled to 0° C. and 2.03 g (9.3 mmol) (BOC)₂O is added. The ice-bath is removed after 15 min and stirring is continued for 2 h at rt. The reaction is quenched by addition of aq. bicarbonate and extracted with DCM. The organic phase is washed with brine, dried with Na₂SO₄, filtered and the solvent is removed in vacuo. The residue is purified by FC on silica (eluent: CH₂Cl₂/MeOH 99:1) to give the title compound as a yellow oil.

TLC, Rf (hexane/EtOAc 1:1)=0.86

MS (method D): 216 [M⁺-55]

### Step 3

### (tert-Butoxycarbonyl-cyclopentylmethyl-amino)-acetic acid

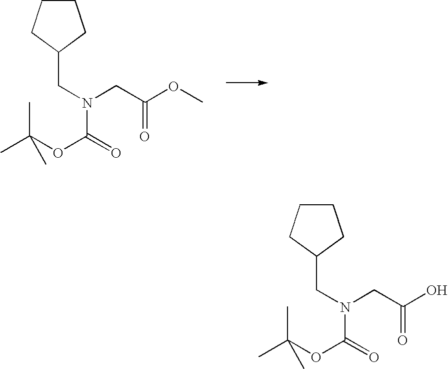

To a solution of 1.22 g (4.5 mmol) (tert-Butoxycarbonyl-cyclopentylmethyl-amino)-acetic acid methyl ester in 40 mL THF/MeOH/H₂O (2:1:1) is added 0.57 g (13.5 mmol) LiOH and the reaction stirred for 15 h at rt. The solvent is removed in vacuo, pH 3 is adjusted by addition of 4N HCl followed by extraction with EtOAc. The combined organic phase is washed with brine, dried with Na₂SO₄, filtered and the solvent is removed in vacuo. The residue is purified by FC on silica (eluent: CH₂Cl₂/MeOH 98:2) to give the title compound as a yellow oil.

TLC, Rf (CH₂Cl₂/MeOH 19:1)=0.34

MS (method D): 202 [M⁺-55]

### Preparation of 8-[2-({(1R,2S)-1-[2-(Cyclopentylmethyl-amino)-acetylamino]-2-vinyl-cyclopro-panecarbonyl}-sulfamoyl)-phenylcarbamoyl]-octanoic acid

### Step 1

### 8-[2-({(1R,2S)-1-[2-(tert-Butoxycarbonyl-cyclopentylmethyl-amino)-acetylamino]-2-vinyl-cyclopropanecarbonyl}-sulfamoyl)-phenylcarbamoyl]-octanoic acid methyl ester

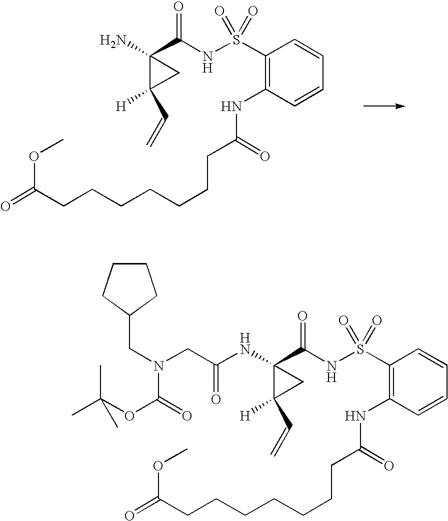

The title compound is prepared analogously as described for the title compound in Example 1 (step 4) using 150 mg (0.19 mmol) 8-{2-[((1R,2S)-1-Amino-2-vinyl-cyclopropanecarbonyl)-sulfamoyl]-phenyl-carbamoyl]-octanoic acid methyl ester (HCl-salt), 57 mg (0.22 mmol) (tert-Butoxycarbonyl-cyclopentylmethyl-amino)-acetic acid, 84 mg (0.22 mmol) HBTU and 120 mg (0.93 mmol) DIPEA in 2 mL DMF.

HPLC (method A) t_{R}=5.98 min

TLC, Rf (CH₂Cl₂/MeOH 19:1)=0.30

MS (method D): 705 [M+]

### Step 2

### 8-[2-({(1R,2S)-1-[2-(Cyclopentylmethyl-amino)-acetylamino]-2-vinyl-cyclopropanecarbonyl}-sulfamoyl)-phenylcarbamoyl]-octanoic acid

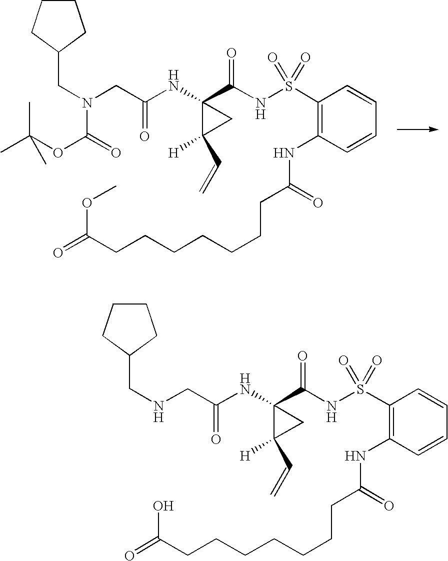

The title compound is prepared analogously as described for the title compound in Example 1 (step 5) using 102 mg (0.14 mmol) 8-[2-({(1R,2S)-1-[2-(tert-Butoxycarbonyl-cyclopentylmethyl-amino)-acetylamino]-2-vinyl-cyclopropanecarbonyl}-sulfamoyl)-phenylcarbamoyl]-octanoic acid methyl ester and 1 mL TFA in 10 mL DCM and 33 mg (1.4 mmol) LiOH in 12 mL THF/MeOH/H₂O (2:1:1).

HPLC (method A) t_{R}=3.99 min

TLC, Rf (CH₂Cl₂/MeOH 85:15)=0.57

MS (method D): 591 [M+]

### Example 7

### (8S,10R)-10-(7-Methoxy-2-phenyl-quinolin-4-yloxy)-5-[(1R,2S)-1-carbonylamino-2-vinyl-cyclopropyl]-2,2-dioxo-2λ*6*-thia-3,6,12,23-tetraaza-tricyclo[22.4.0.0*8,121octacosa-1(28),24,26-triene-4,7,13,22-tetraone

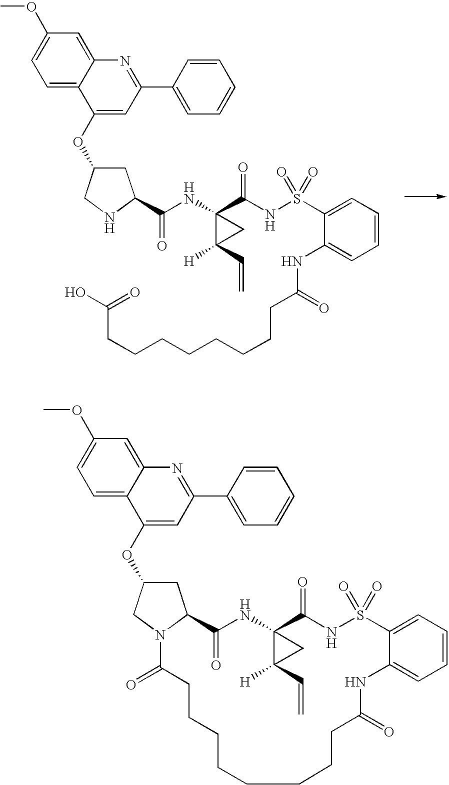

The title compound is prepared analogously as described for the title compound in Example 2 using 150 mg (0.16 mmol) 9-{2-[((1R,2S)-1-{[(2S,4R)-4-(7-Methoxy-2-phenyl-quinolin-4-yloxy)-pyrrolidine-2-carbonyl]-amino}-2-vinyl-cyclopropanecarbonyl)-sulfamoyl]-phenylcarbamoyl}-nonanoic acid (TFA-salt), 207 mg (1.6 mmol) DIPEA and 304 mg (0.80 mmol) HATU in 51 mL DCM/MeOH (50:1).

HPLC (method A) t_{R}=5.00 min

TLC, Rf (CH₂Cl₂/MeOH 9:1)=0.5

MS (method D): 794 [M+]

### Preparation of (8-{2-[((1R,2S)-1-{[(2S,4R)-4-(7-Methoxy-2-phenyl-quinolin-4-yloxy)-pyrrolidine-2-carbonyl]-amino}-2-vinyl-cyclopropanecarbonyl)-sulfamoyl]-phenylcarbamoyl}-octanoic acid

### Step 1

### 9-{2-[((1R,2S)-1-tert-Butoxycarbonylamino-2-vinyl-cyclopropanecarbonyl)-sulfamoyl]-phenylcarbamoyl}-nonanoic acid methyl ester

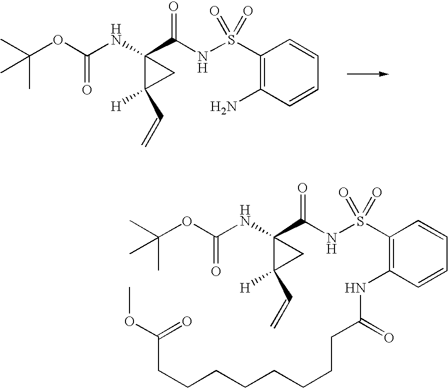

The title compound is prepared analogously as described for the title compound in Example 1 (Step 2) using 1.50 g (3.9 mmol) [(1R,2S)-1-(2-Amino-benzenesulfonylaminocarbonyl)-2-vinyl-cyclopropyl]-carbamic acid tert-butyl ester, 2.12 g (9.8 mmol) Monomethyl sebacate, 1.41 g (11.8 mmol) Benzotriazole, 1.41 g (11.8 mmol) Thionylchloride, 1.84 g (20.0 mmol) NEt₃ and 100 mg DMAP in 50 mL DCM.

HPLC (method A) t_{R}=5.42 min

TLC, Rf (CH₂Cl₂/MeOH 19:1)=0.33

MS (method D): 580 [M+]

### Step 2

### 9-{2-[((1R,2S)-1-Amino-2-vinyl-cyclopropanecarbonyl)-sulfamoyl]-phenylcarbamoyl}-nonanoic acid methyl ester

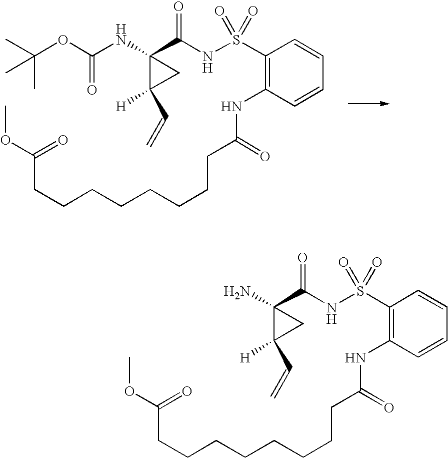

The title compound is prepared analogously as described for the title compound in Example 1 (Step 3) using 1.10 g (1.9 mmol) 9-{2-[((1R,2S)-1-tert-Butoxycarbonylamino-2-vinyl-cyclopropanecarbonyl)-sulfamoyl]-phenylcarbamoyl}-nonanoic acid methyl ester and 3 mL 4 N HCl in Dioxane.

HPLC (method A) t_{R}=3.65 min

MS (method D): 480 [M+]

### Step 3

### (2S,4R)-2-{(1R,2S)-1-[2-(9-Methoxycarbonyl-nonanoylamino)-benzenesulfonylaminocarbonyl]-2-vinyl-cyclopropylcarbamoyl}-4-(7-methoxy-2-phenyl-quinolin-4-yloxy)-pyrrolidine-1-carboxylic acid tert-butyl ester

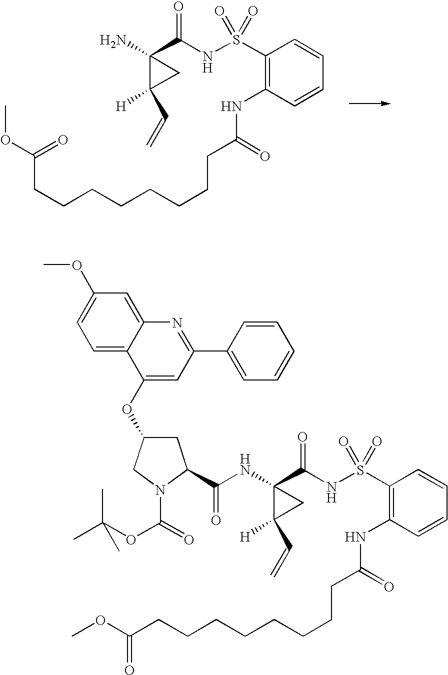

The title compound is prepared analogously as described for the title compound in Example 1 (Step 4) using 280 mg (0.43 mmol) 9-{2-[((1R,2S)-1-Amino-2-vinyl-cyclopropanecarbonyl)-sulfamoyl]-phenylcarbamoyl}-nonanoic acid methyl ester (HCl-salt), 218 mg (0.47 mmol) (2S,4R)-4-(7-Methoxy-2-phenyl-quinolin-4-yloxy)-pyrrolidine-1,2-dicarboxylic acid 1-tert-butyl ester, 278 mg (2.15 mmol) DIPEA and 212 mg (0.56 mmol) HBTU in 2 mL DMF.

HPLC (method A) t_{R}=5.59 min

TLC, Rf (CH₂Cl₂/MeOH 19:1)=0.23

MS (method D): 926 [M+]

### Step 4

### (2S,4R)-2-{(1R,2S)-1-[2-(9-Carboxy-nonanoylamino)-benzenesulfonylaminocarbonyl]-2-vinyl-cyclopropylcarbamoyl}-4-(7-methoxy-2-phenyl-quinolin-4-yloxy)-pyrrolidine-1-carboxylic acid tert-butyl ester

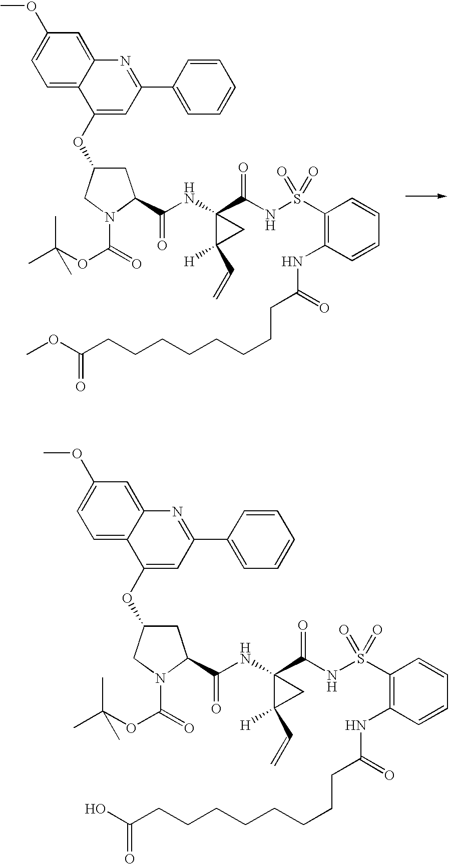

The title compound is prepared analogously as described for the title compound in Example 2 (Step 2) using 152 mg (0.16 mmol) (2S,4R)-2-{(1R,2S)-1-[2-(9-Methoxycarbonyl-nonanoylamino)-benzene-sulfonylaminocarbonyl]-2-vinyl-cyclopropylcarbamoyl}-4-(7-methoxy-2-phenyl-quinolin-4-yloxy)-pyrrolidine-1-carboxylic acid tert-butyl ester and 38 mg (1.6 mmol) LiOH in 8 mL THF/MeOH/H₂O (2:1:1).

HPLC (method A) t_{R}=5.06 min

MS (method D): 912 [M+]

### Step 5

### 9-{2-[((1R,2S)-1-{[(2S,4R)-4-(7-Methoxy-2-phenyl-quinolin-4-yloxy)-pyrrolidine-2-carbonyl]-amino}-2-vinyl-cyclopropanecarbonyl)-sulfamoyl]-phenylcarbamoyl}-nonanoic acid

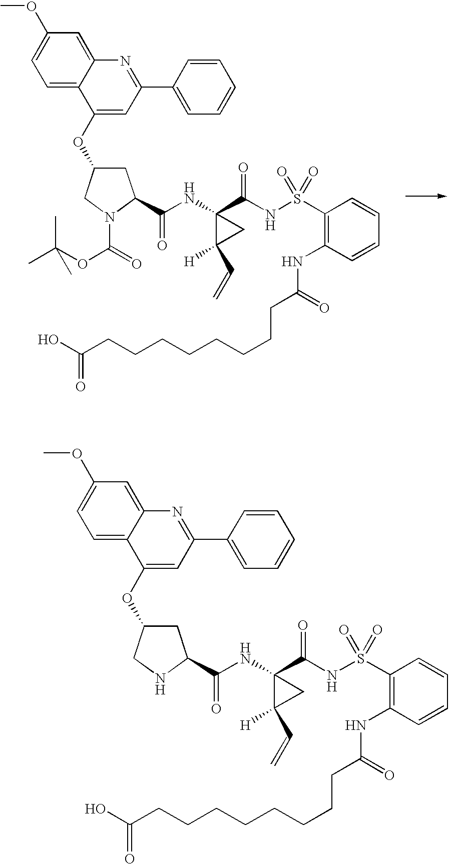

The title compound is prepared analogously as described for the title compound in Example 2 (Step 3) using 150 mg (0.16 mmol) (2S,4R)-2-{(1R,2S)-1-[2-(9-Carboxy-nonanoylamino-benzene-sulfonylaminocarbonyl]-2-vinyl-cyclopropylcarbamoyl}-4-(7-methoxy-2-phenyl-quinolin-4-yloxy)-pyrrolidine-1-carboxylic acid tert-butyl ester and 1 mL TFA in 5 mL DCM.

HPLC (method A) t_{R}=4.61 min

MS (method D): 812 [M+]

### Example 8

### (8S,10R)-10-[2-(2-Isopropylamino-thiazol-4-yl)-7-methoxy-quinolin-4-yloxy]-5-[(1R,2S)-1-carbonylamino-2-vinyl-cyclopropyl]-2,2-dioxo-2λ*6*-thia-3,6,12,23-tetraaza-tricyclo[22.4.0.0*8,12]octacosa-1(28),24,26-triene-4,7,13,22-tetraone

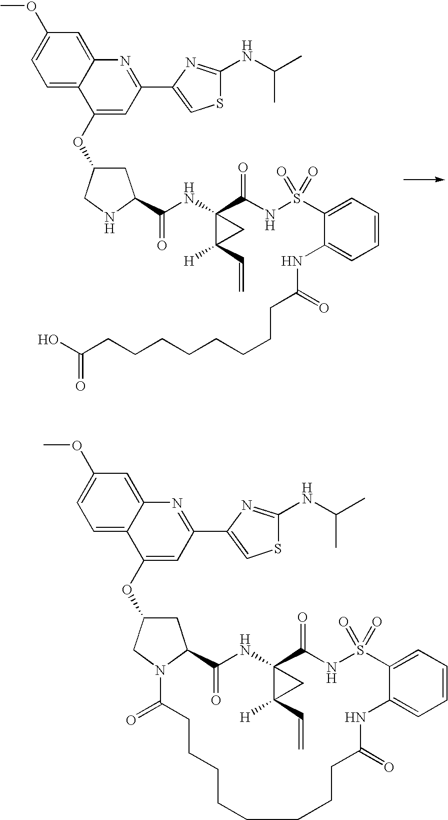

The title compound is prepared analogously as described for the title compound in Example 2 using 57 mg (0.05 mmol) 9-(2-{[(1R,2S)-1-({(2S,4R)-4-[2-(2-Isopropylamino-thiazol-4-yl)-7-methoxy-quinolin-4-yloxy]-pyrrolidine-2-carbonyl}-amino)-2-vinyl-cyclopropanecarbonyl]-sulfamoyl)-phenylcarbamoyl)-nonanoic acid, 67 mg (0.52 mmol)

DIPEA and 99 mg (0.26 mmol) HATU in 51 mL DCM/DMF (50:1).

HPLC (method A) t_{R}=5.33 min

TLC, Rf (CH₂Cl₂/MeOH 9:1)=0.30

MS (method D): 858 [M+]

### Preparation of 9-(2-{[(1R,2S)-1-({(2S,4R)-4-[2-(2-Isopropylamino-thiazol-4-yl)-7-methoxy-quinolin-4-yloxy]-pyrrolidine-2-carbonyl}-amino)-2-vinyl-cyclopropanecarbonyl]-sulfamoyl}-phenylcarbamoyl)-nonanoic acid

### Step 1

### (2S,4R)-4-[2-(2-Isopropyl-thiazol-4-yl)-7-methoxy-quinolin-4-yloxy]-2-{(1R,2S)-1-[2-(8-methoxycarbonyl-octanoylamino)-benzenesulfonylaminocarbonyl]-2-vinyl-cyclopropylcarbamoyl}-pyrrolidine-1-carboxylic acid tert-butyl ester

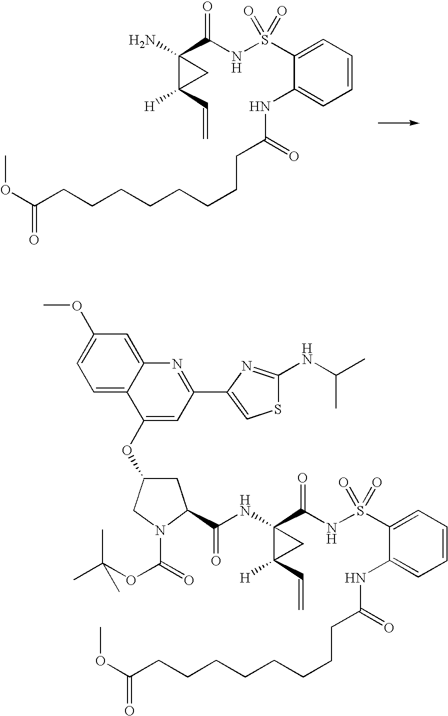

The title compound is prepared analogously as described for the title compound in Example 2 (step 1) using 150 mg (0.22 mmol) 9- {2-[((1R,2S)-1-Amino-2-vinyl-cyclopropanecarbonyl)-sulfamoyl]-phenylcarbamoyl}-nonanoic acid methyl ester, 117 mg (0.22 mmol) (2S,4R)-4-[2-(2-Isopropylamino-thiazol-4-yl)-7-methoxy-quinolin-4-yloxy]-pyrrolidine-1,2-dicarboxylic acid 1-tert-butyl ester, 101 mg (0.27 mmol) HATU and 143 mg (1.1 mmol) DIPEA in 5 mL DMF.

HPLC (method A) t_{R}=5.80 min

TLC, Rf (CH₂Cl₂/MeOH 9:1)=0.30

MS (method D): 990 [M+]

### Step 2

### (2S,4R)-2-{(1R,2S)-1-[2-(9-Carboxy-nonanoylamino)-benzenesulfonylaminocarbonyl]-2vinyl-cyclopropylcarbamoyl}-4-[2-(2-isopropylamino-thiazol-4-yl)-7-methoxy-quinolin-4-yloxy]-pyrrolidine-1-carboxylic acid tert-butyl ester

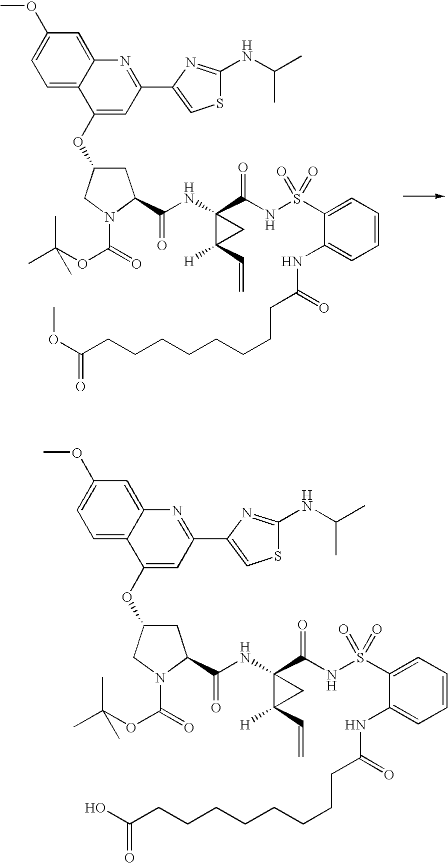

The title compound is prepared analogously as described for the title compound in Example 2 (step 2) using 59 mg (0.053 mmol) (2S,4R)-4-[2-(2-Isopropyl-thiazol-4-yl)-7-methoxy-quinolin-4-yloxy]-2- {(1R,2S)-1-[2-(8-methoxycarbonyl-octanoylamino)-benzenesulfonylaminocarbonyl]-2-vinyl-cyclopropyl-carbamoyl}-pyrrolidine-1-carboxylic acid tert-butyl ester and 22 mg (0.53 mmol) LiOH in 8 mL THF/MeOH/H₂O (2:1:1).

HPLC (method A) t_{R}=5.28 min

TLC, Rf (CH₂Cl₂/MeOH 9:1)=0.26

MS (method D): 976 [M+]

### Step 3

### 9-(2-{[(1R,2S)-1-({(2S,4R)-4-[2-(2-Isopropylamino-thiazol-4-yl)-7-methoxy-quinolin-4-yloxy]-pyrrolidine-2-carbonyl}-amino)-2-vinyl-cyclopropanecarbonyl]-sulfamoyl}-phenylcarbamoyl)-nonanoic acid

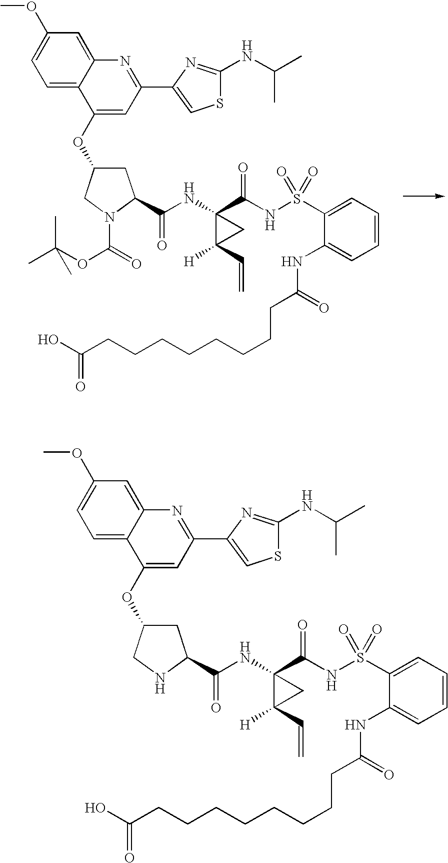

The title compound is prepared analogously as described for the title compound in Example 2 (step 3) using 50 mg (0.051 mmol) (2S,4R)-2-{(1R,2S)-1-[2-(9-Carboxy-nonanoylamino)-benzenesulfonyl-aminocarbonyl]-2-vinyl-cyclopropylcarbamoyl}-4-[2-(2-isopropylamino-thiazol-4-yl)-7-methoxy-quinolin-4-yloxy]-pyrrolidine-1-carboxylic acid tert-butyl ester and 0.5 mL TFA in 5 mL DCM.

HPLC (method A) t_{R}=4.74 min

TLC, Rf (CH₂Cl₂/MeOH/H₂O/AcOH 90:10:1:0.5)=0.16

MS (method D): 876 [M+]

### Example 9

### (8S,10R)-10-(7-Methoxy-2-phenyl-quinolin-4-yloxy)-5-[(1R,2S)-1-carbonylamino-2-vinyl-cyclopropyl]-2,2-dioxo-2λ*6*-thia-3,6,12,21-tetraaza-tricyclo[20.4.0.0*8,12*]hexacosa-1(26),22,24-triene-4,7,13,20-tetraone

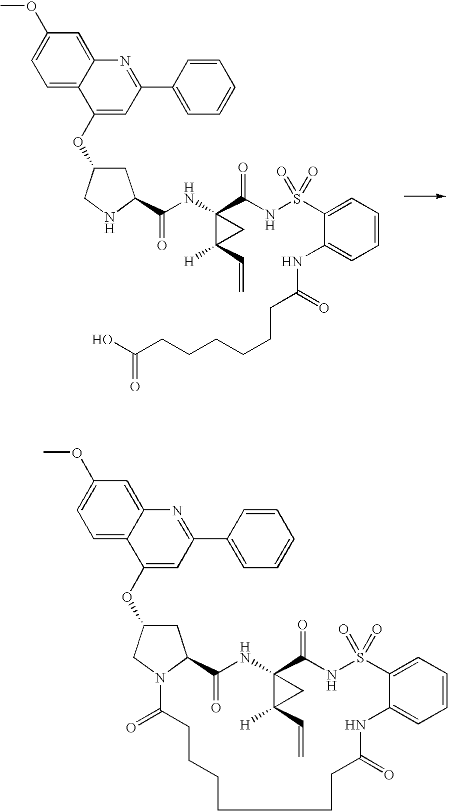

The title compound is prepared analogously as described for the title compound in Example 2 using 121 mg (0.14 mmol) 7-{2-[((1R,2S)-1-{[(2S,4R)-4-(7-Methoxy-2-phenyl-quinolin-4-yloxy)-pyrroidine-2-carbonyl]-amino}-2-vinyl-cyclopropanecarbonyl)-sulfamoyl]-phenylcarbamoyl}-heptanoic acid (TFA-salt), 174 mg (1.4 mmol) DIPEA and 257 mg (0.66 mmol) HATU in 51 mL DCM/DMF (50:1).

HPLC (method A) t_{R}=4.68 min

TLC, Rf (CH₂Cl₂/MeOH 85:15)=0.43

MS (method D): 766 [M+]

### Preparation of 7-{2-[((1R,2S)-1-{[(2S,4R)-4-(7-Methoxy-2-phenyl-quinolin-4-yloxy)-pyrrolidine-2-carbonyl]-amino}-2-vinyl-cyclopropanecarbonyl)-sulfamoyl]-phenylcarbamoyl}-heptanoic acid

### Step 1

### 7-{2-[((1R,2S)-1-tert-Butoxycarbonylamino-2-vinyl-cyclopropanecarbonyl)-sulfamoyl]-phenylcarbamoyl}-heptanoic acid methyl ester

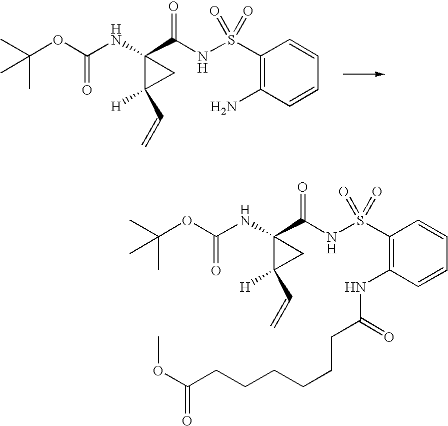

The title compound is prepared analogously as described for the title compound in Example 1 (Step 2) using 0.76 g (1.99 mmol) [(1R,2S)-1-(2-Amino-benzenesulfonylaminocarbonyl)-2-vinyl-cyclopropyl]-carbamic acid tert-butyl ester, 0.94 g (4.97 mmol) Monomethyl suberate, 0.71 g (5.97 mmol) Benzotriazole, 0.71 g (5.97 mmol) Thionylchloride, 0.92 g (10 mmol) NEt₃ and 70 mg DMAP in 40 mL DCM.

HPLC (method A) t_{R}=4.95 min

TLC, Rf (CH₂Cl₂/MeOH 19:1)=0.23

MS (method D): 552 [M+]

### Step 2

### 7-{2-[((1R,2S)-1-Amino-2-vinyl-cyclopropanecarbonyl)-sulfamoyl]-phenylcarbamoyl}-heptanoic acid methyl ester

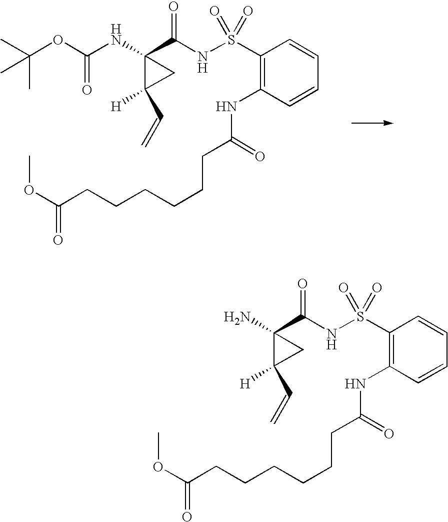

The title compound is prepared analogously as described for the title compound in Example 1 (Step 3) using 0.78 g (1.4 mmol) 7-{(2-[((1R,2S)-1-tert-Butoxycarbonylamino-2-vinyl-cyclopropanecarbonyl)-sulfamoyl]-phenylcarbamoyl}-heptanoic acid methyl ester and 1 mL 4N HCl in Dioxane.

HPLC (method A) t_{R}=3.04 min

MS (method D): 452 [M+]

### Step 3

### (2S,4R)-2-{(1R,2S)-1-[2-(7-Methoxycarbonyl-heptanoylamino)-benzenesulfonylaminocarbonyl]-2-vinyl-cyclopropylcarbamoyl}-4-(7-methoxy-2-phenyl-quinolin-4-yloxy)-pyrrolidine-1-carboxylic acid tert-butyl ester

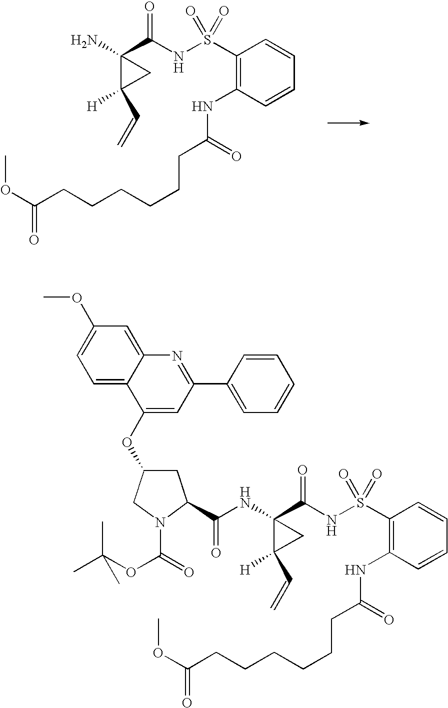

The title compound is prepared analogously as described for the title compound in Example 1 (Step 4) using 150 mg (0.22 mmol) 7-{2-[((1R,2S)-1-Amino-2-vinyl-cyclopropanecarbonyl)-sulfamoyl]-phenylcarbamoyl}-heptanoic acid methyl ester, 120 mg (0.26 mmol) (2S,4R)-4-(7-Methoxy-2-phenyl-quinolin-4-yloxy)-pyrrolidine-1,2-dicarboxylic acid 1-tert-butyl ester, 98 mg (0.26 mmol) HBTU and 139 mg (1.1 mmol) DIPEA in 2 mL DMF.

HPLC (method A) t_{R}=5.19 min

TLC, Rf (CH₂Cl₂/MeOH 19:1)=0.43

MS (method D): 898 [M+]

### Step 4

### (2S,4R)-2-{(1R,2S)-1-[2-(7-Carboxy-heptanoylamino)-benzenesulfonylaminocarbonyl]-2-vinyl-cyclopropylcarbamoyl}-4-(7-methoxy-2-phenyl-quinolin-4-yloxy)-pyrrolidine-1-carboxylic acid tert-butyl ester

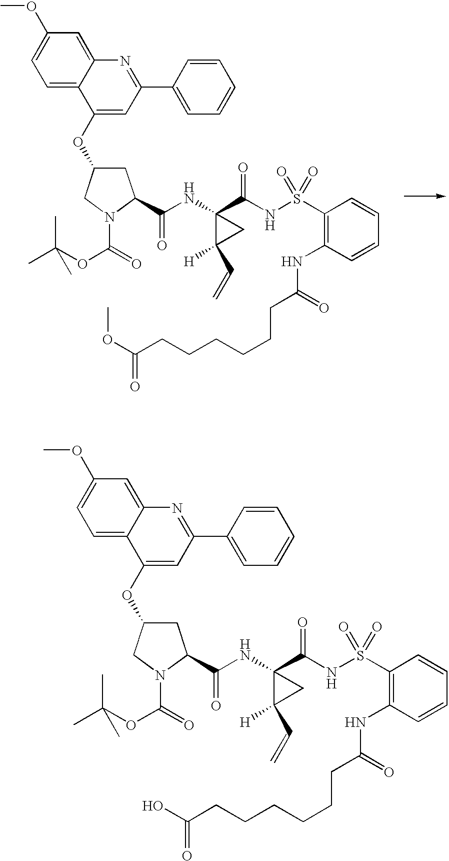

The title compound is prepared analogously as described for the title compound in Example 2 (Step 2) using 179 mg (0.17 mmol) (2S,4R)-2-{(1R,2S)-1-[2-(7-Methoxycarbonyl-heptanoylamino)-benzenesulfonylaminocarbonyl]-2-vinyl-cyclopropylcarbamoyl}-4-(7-methoxy-2-phenyl-quinolin-4-yloxy)-pyrrolidine-1-carboxylic acid tert-butyl ester and 41 in (1.7 mmol) LiOH in 10 mL THF/MeOH/H₂O (2:1:1).

HPLC (method A) t_{R}=4.74 min

TLC, Rf (CH₂Cl₂/MeOH 9:1)=0.32

MS (method D): 884 [M+]

### Step 5

### 7-{2-[((1R,2S)-1-{[(2S,4R)-4-(7-Methoxy-2-phenyl-quinolin-4-yloxy)-pyrrolidine-2-carbonyl]-amino}-2-vinyl-cyclopropanecarbonyl)-sulfamoyl]-phenylcarbamoyl}-heptanoic acid

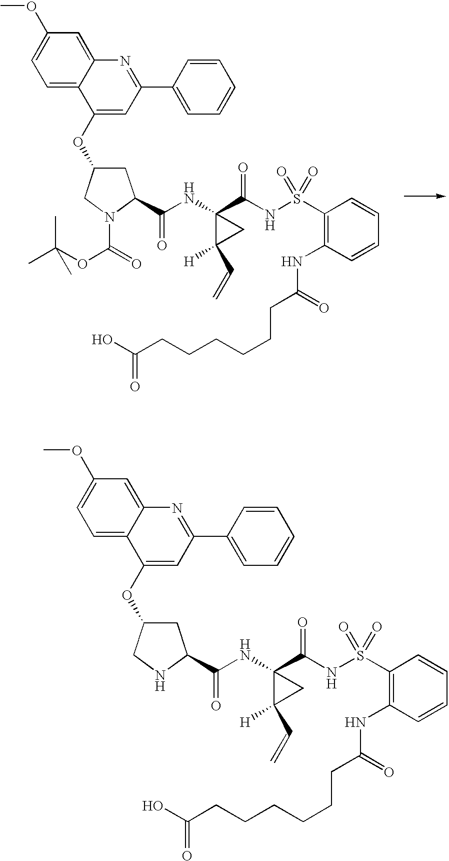

The title compound is prepared analogously as described for the title compound in Example 2 (Step 3) using 134 mg (0.15 mmol) (2S,4R)-2-{(1R,2S)-1-[2-(7-Carboxy-heptanoylamino)-benzenesulfonyl-aminocarbonyl]-2-vinyl-cyclopropylcarbamoyl}-4-(7-methoxy-2-phenyl-quinolin-4-yloxy)-pyrrolidine-1-carboxylic acid tert-butyl ester and 1 mL TFA in 25 mL DCM.

HPLC (method A) t_{R}=4.04 min

TLC, Rf (CH₂Cl₂/MeOH 85:15)=0.54

MS (method D): 784 [M+]

### Example 10

### (8S,10R)-10-[2-(2-Isopropylamino-thiazol-4-yl)-7-methoxy-quinolin-4-yloxy]-5-[(1R,2S)-1-carbonylamino-2-vinyl-cyclopropyl]-2,2-dioxo-2λ*6*-thia-3,6,12,21-tetraaza-tricyclo[20.4.0.0*8,12*]hexacosa-1(26),22,24-triene-4,7,13,20-tetraone

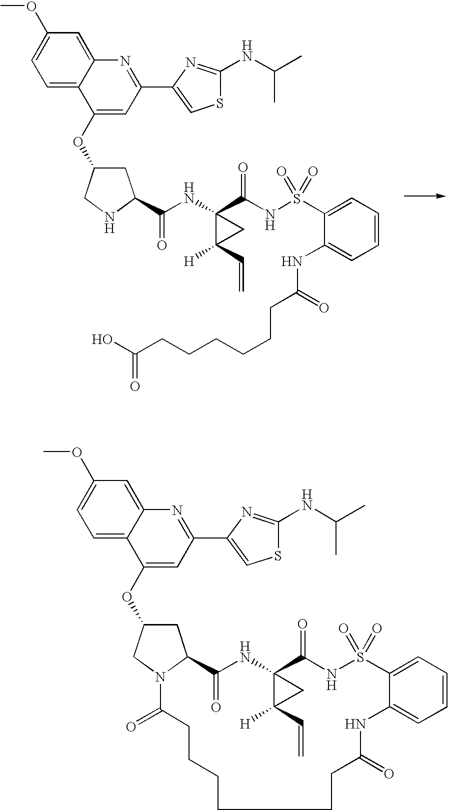

The title compound is prepared analogously as described for the title compound in Example 2 using 121 mg (0.11 mmol) 7-(2-{[(1R,2S)-1-({(2S,4R)-4-[2-(2-Isopropylamino-thiazol-4-yl)-7-methoxy-quinolin-4-yloxy]-pyrrolidine-2-carbonyl}-amino)-2-vinyl-cyclopropanecarbonyl]sulfamoyl}-phenyl-carbamoyl)-heptanoic acid (TFA-salt), 145 mg (1.1 mmol) DIPEA and 213 mg (0.56 mmol) HATU in 51 mL DCM/DMF (50:1).

HPLC (method A) t_{R}=4.98 min

TLC, Rf (CH₂Cl₂/MeOH 85:15)=0.46

MS (method D): 830 [M+]

### Preparation of 7-(2-{[(1R,2S)-1-({(2S,4R)-4-[2-(2-Isopropylamino-thiazol-4-yl)-7-methoxy-quinolin-4-yloxy]-pyrrolidine-2-carbonyl}-amino)-2-vinyl-cyclopropanecarbonyl]-sulfamoyl}-phenylcarbamoyl)-heptanoic acid

### Step 1

### (2S,4R)-4-[2-(2-Isopropylamino-thiazol-4-yl)-7-methoxy-quinolin-4-yloxy]-2-{(1R,2S)-1-[2-(7-methoxycarbonyl-heptanoylamino)-benzenesulfonylaminocarbonyl]-2-vinyl-cyclopropylcarbamoyl}-pyrrolidine-1-carboxylic acid tert-butyl ester

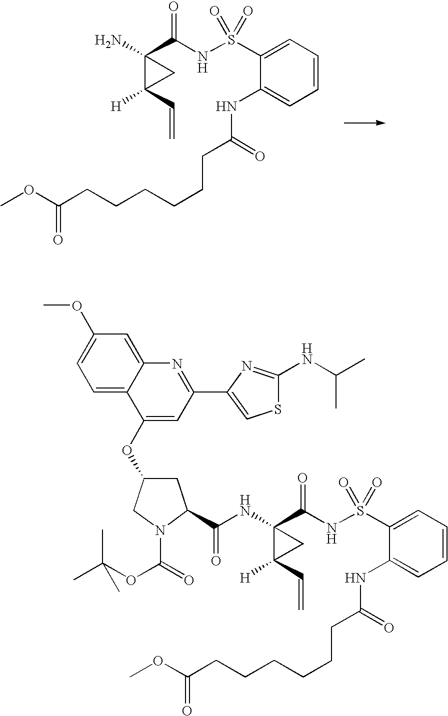

The title compound is prepared analogously as described for the title compound in Example 2 (step 1) using 170 mg (0.24 mmol) 7-{2-[((1R,2S)-1-Amino-2-vinyl-cyclopropanecarbonyl)-sulfamoyl]-phenylcarbamoyl}-heptanoic acid methyl ester (HCl-salt), 207 mg (0.29 mmol) (2S,4R)-4-[2-(2-Isopropylamino-thiazol-4-yl)-7-methoxy-quinolin-4-yloxy]-pyrrolidine-1,2-dicarboxylic acid 1-tert-butyl ester, 111 mg (0.29 mmol) HBTU and 158 mg (1.2 mmol) DIPEA in 2 mL DMF.

HPLC (method A) t_{R}=5.35 min

TLC, Rf (CH₂Cl₂/MeOH 19:1)=0.27

MS (method D): 962 [M+]

### Step 2

### (2S,4R)-2-{(1R,25)-1-[2-(7-Carboxy-heptanoylamino)-benzenesulfonylaminocarbonyl]-2-vinyl-cyclopropylcarbamoyl}-4-[2-(2-isopropylamino-thiazol-4-yl)-7-methoxy-quinolin-4-yloxy]-pyrrolidine-1-carboxylic acid tert-butyl ester

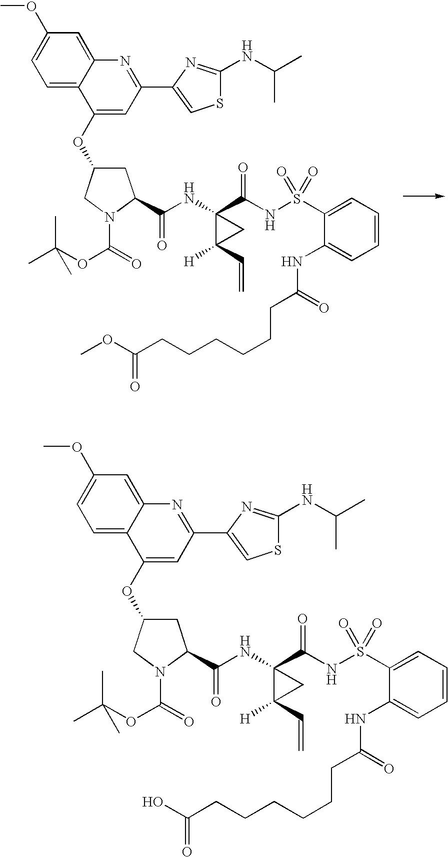

The title compound is prepared analogously as described for the title compound in Example 2 (step 2) using 138 mg (0.14 mmol) (2S,4R)-4-[2-(2-Isopropylamino-thiazol-4-yl)-7-methoxy-quinolin-4-yloxy]-2-{(1R,2S)-1-[2-(7-methoxycarbonyl-heptanoylamino)-benzenesulfonylaminocarbonyl]-2-vinyl-cyclo-propylcarbamoyl}-pyrrolidine-1-carboxylic acid tert-butyl ester and 35 mg (1.4 mmol) LiOH in 10 mL THF/MeOH/H₂O (2:1:1).

HPLC (method A) t_{R}=5.07 min

TLC, Rf (CH₂Cl₂/MeOH 85:15)=0.55

MS (method D): 948 [M+]

### Step 3

### 8-[2-({(1R,2S)-1-[(2-Amino-indane-2-carbonyl)-amino]-2-vinyl-cyclopropanecarbonyl}-sulfamoyl)-phenylcarbamoyl]-octanoic acid

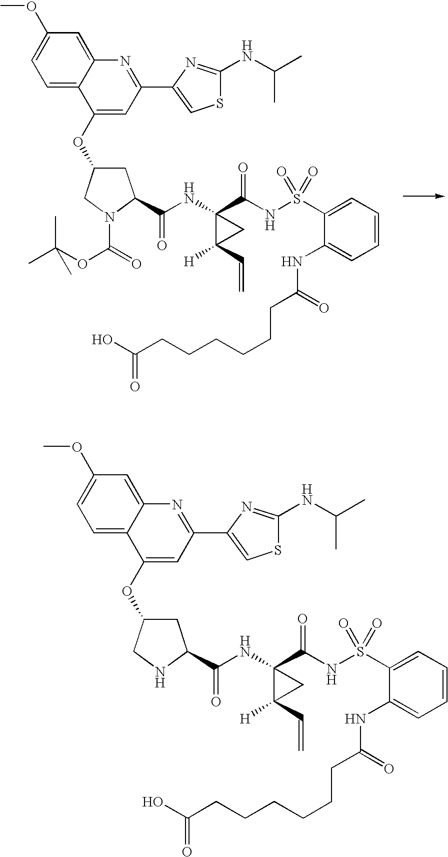

The title compound is prepared analogously as described for the title compound in Example 2 (step 3) using 135 mg (0.14 mmol) (2S,4R)-2-{(1R,2S)-1-[2-(7-Carboxy-heptanoamino)-benzenesulfonyl-aminocarbonyl]-2-vinyl-cyclopropylcarbamoyl}-4-[2-(2-isopropylamino-thiazol-4-yl)-7-methoxy-quinolin-4-yloxy]-pyrrolidine-1-carboxylic acid tert-butyl ester and 1 mL TFA in 25 mL DCM.

HPLC (method A) t_{R}=4.33 min

TLC, Rf (CH₂Cl₂/MeOH 85:15)=0.46

MS (method D): 848 [M+]

### Example 11

### (8S,10R)-10-(7-Methoxy-2-phenyl-quinolin-4-yloxy)-5-[(1R,2S)-1-carbonylamino-2-vinyl-cyclopropyl]-2,2-dioxo-2λ*6*-thia-3,6,12,22-tetraaza-tricyclo[21.4.0.0*8,12*]heptacosa-1(27),23,25-triene-4,7,13-trione

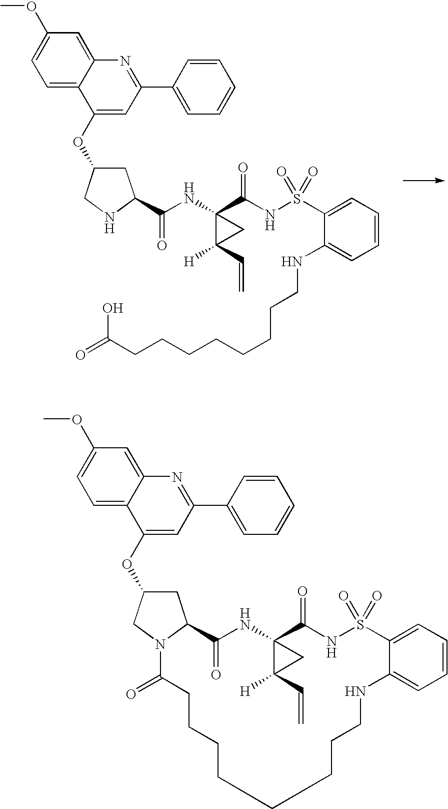

The title compound is prepared analogously as described for the title compound in Example 2 using 80 mg (0.08 mmol) 9-{2-[((1R,2S)-1-{[(2S,4R)-4-(7-Methoxy-2-phenyl-quinolin-4-yloxy)-pyrrolidine-2-carbonyl]-amino}-2-vinyl-cyclopropanecarbonyl)-sulfamoyl]-phenylamino}-nonanoic acid (TFA-salt), 102 mg (0.80 mmol) DIPEA and 150 mg (0.40 mmol) HATU in 25 mL DCM and 0.5 mL DMF.

HPLC (method A) t_{R}=5.43 min

TLC, Rf (CH₂Cl₂/MeOH 19:1)=0.37

MS (method D): 766 [M+H₂O]

### Preparation of 9-{2-[((1R,2S)-1-{[(2S,4R)-4-(7-Methoxy-2-phenyl-quinolin-4-yloxy)-pyrrolidine-2-carbonyl]-amino}-2-vinyl-cyclopropanecarbonyl)-sulfamoyl]-phenylamino}-nonanoic acid

### Step 1

### 9-Hydroxy-nonanoic acid methyl ester

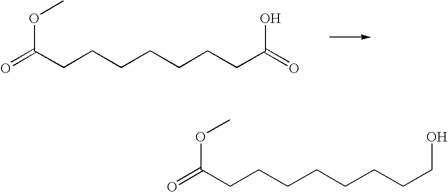

To an ice-cold solution of 10.0 g (45 mmol) Mono-methyl azelate in 250 mL THF is added 90 mL (90 mmol) BH₃*THF-Komplex (1M in THF), the ice-bath is removed and stirring is continued at rt for 90 min. The reaction is quenched by careful addition of Methanol, the main solvent is evaporated, the residue is diluted with water and extracted with EtOAc. The combined organic phase is dried with Na₂SO₄, filtered, and the solvent is removed in vacuo to give the title compound as a colorless oil, which is used without further purification.

MS (method D): 206 [M+H₂O]

### Step 2

### 9-Oxo-nonanoic acid methyl ester

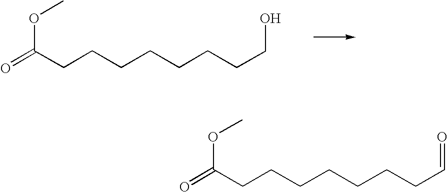

To a solution of 5.2 g (28 mmol) 9-Hydroxy-nonanoic acid methyl ester in 350 mL DCM is added 9.1 g (41 mmol) Pyridinium chlorochromate and the reaction is stirred for 15 h at rt. The reaction is diluted with DCM, silica is added, the mixture is filtered through a pad of Hyflo and thoroughly washed with DCM. The solvent is removed in vacuo to give the title compound as a green oil, which is used without further purification.

MS (method D): 204 [M+H₂O]

### Step 3

### 9-{2-[((1R,2S)-1-tert-Butoxycarbonylamino-2-vinyl-cyclopropanecarbonyl)-sulfamoyl]-phenylamino}-nonanoic acid methyl ester

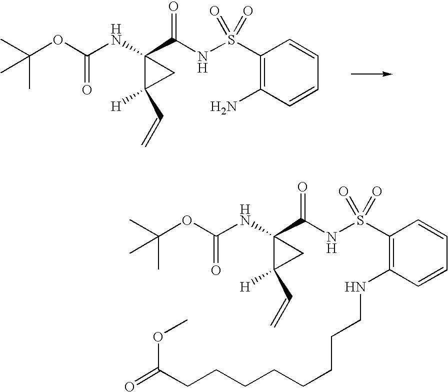

To a solution of 100 mg (0.26 mmol) [(1R,2S)-1-(2-Amino-benzenesulfonylaminocarbonyl)-2-vinyl-cyclopropyl]-carbamic acid tert-butyl ester and 98 mg (0.52 mmol) 9-Oxo-nonanoic acid methyl ester in 15 mL 1,2-Dichloroethane is added at rt 0.045 mL (0.79 mmol) AcOH followed by 145 mg (0.67 mmol) NaBH(OAc)₃. After stirring for 15 h at rt the solvent is removed in vacuo and the residue is purified by preparative reverse phase HPLC (Method G) to give the title compound as a yellow oil.

HPLC (method A) t_{R}=5.68 min

MS (method D): 552 [M+]

### Step 4

### 9-{2-[((1R,2S)-1-Amino-2-vinyl-cyclopropanecarbonyl)-sulfamoyl]-phenylamino}-nonanoic acid methyl ester

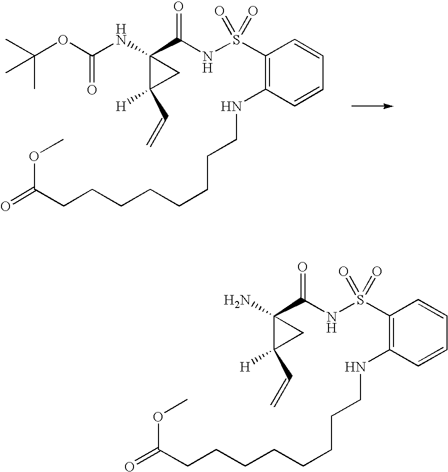

To a solution of 2.10 g (1.56 mmol) 9-{2-[((1R,2S)-1-tert-Butoxycarbonylamino-2-vinyl-cyclopropane-carbonyl)-sulfamoyl]-phenylamino}-nonanoic acid methyl ester in 50 mL Dioxane is added 25 mL 4N HCl in Dioxane and the reaction is stirred for 15 h at rt. The solvent is removed in vacuo and the residue is purified by preparative reverse phase HPLC (Method G) to give the title compound as an orange oil.

HPLC (method A) t_{R}=4.00 min

TLC, Rf (CH₂Cl₂/MeOH 19:1)=0.38

MS (method D): 452 [M+]

### Step 5

### (2S,4R)-2-{(1R,2S)-1-[2-(8-Methoxycarbonyl-octylamino)-benzenesulfonylaminocarbonyl]-2-vinyl-cyclopropylcarbamoyl}-4-(7-methoxy-2-phenyl-quinolin-4-yloxy)-pyrrolidine-1-carboxylic acid tert-butyl ester

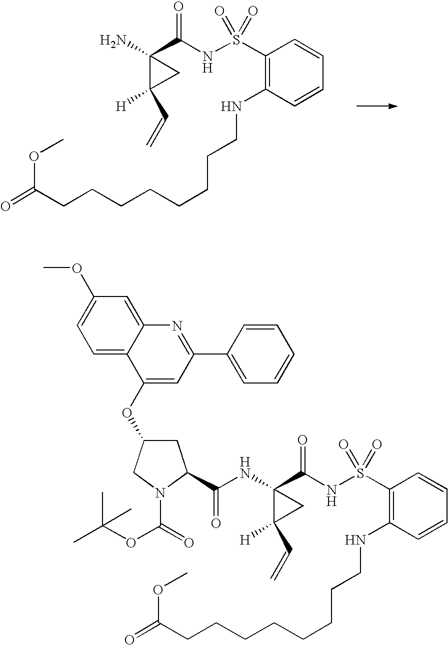

The title compound is prepared analogously as described for the title compound in Example 2 (step 1) using 105 mg (0.21 mmol) 9-{2-[((1R,2S)-1-Amino-2-vinyl-cyclopropanecarbonyl)-sulfamoyl]-phenylamino}-nonanoic acid methyl ester, 95 mg (0.21 mmol) (2S,4R)-4-(7-Methoxy-2-phenyl-quinolin-4-yloxy)-pyrrolidine-1,2-dicarboxylic acid 1-tert-butyl ester, 102 mg (0.27 mmol) HATU and 133 mg (1.0 mmol) DIPEA in 5 mL DMF.

HPLC (method A) t_{R}=5.83 min

MS (method D): 898 [M+]

### Step 6

### (2S,4R)-2-{(1R,2S)-1-[2-(8-Carboxy-octylamino)-benzenesulfonylaminocarbonyl]-2-vinyl-cyclopropylcarbamoyl}-4-(7-methoxy-2-phenyl-quinolin-4-yloxy)-pyrrolidine-1-carboxylic acid tert-butyl ester

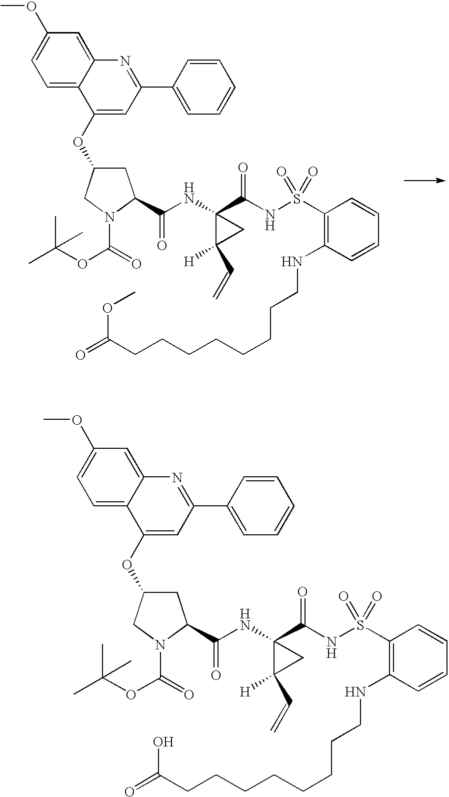

The title compound is prepared analogously as described for the title compound in Example 2 (step 2) using 73 mg (0.08 mmol) (2S,4R)-2-{(1R,2S)-1-[2-(8-Methoxycarbonyl-octylamino)-benzenesulfonyl-aminocarbonyl]-2-vinyl-cyclopropylcarbamoyl}-4-(7-methoxy-2-phenyl-quinolin-4-yloxy)-pyrrolidine-1-carboxylic acid tert-butyl ester and 20 mg LiOH in 8 mL THF/MeOH/H2O (2:1:1).

HPLC (method A) t_{R}=5.29 min

TLC, Rf (CH₂Cl₂/MeOH/H₂O/AcOH 90:10:1:0.5)=0.66

MS (method D): 884 [M+]

### Step 7

### 9-{2-[((1R,2S)-1-{[(2S,4R)-4-(7-Methoxy-2-phenyl-quinolin-4-yloxy)-pyrrolidine-2-carbonyl]-amino}-2-vinyl-cyclopropanecarbonyl)-sulfamoyl]-phenylamino}-nonanoic acid

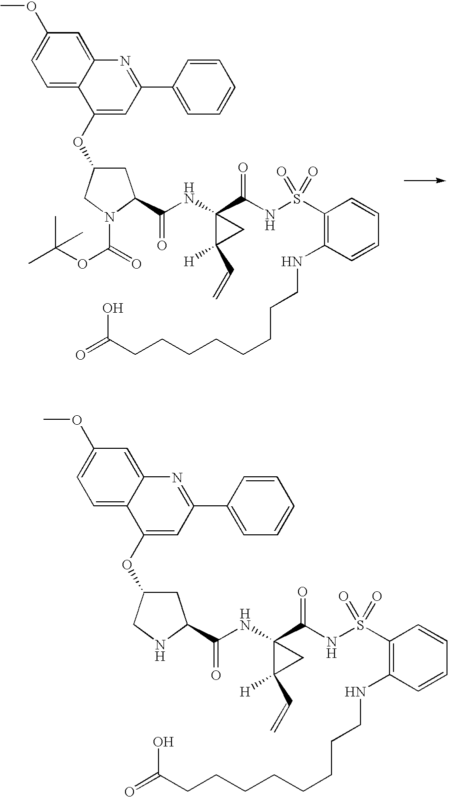

The title compound is prepared analogously as described for the title compound in Example 2 (step 3) using 71 mg (0.08 mmol) (2S,4R)-2-{(1R,2S)-142-(8-Carboxy-octylamino)-benzenesulfonyl-amino-carbonyl]-2-vinyl-cyclopropylcarbamoyl}-4-(7-methoxy-2-phenyl-quinolin-4-yloxy)-pyrrolidine-1-carboxylic acid tert-butyl ester and 0.3 mL TFA in 5 mL DCM.

HPLC (method A) t_{R}=4.78 min

TLC, Rf (CH₂Cl₂/MeOH/H₂O/AcOH 90:10:1:0.5)=0.41

MS (method D): 784 [M+]

### Example 12

### (8S,10R)-10-[2-(2-Isopropylamino-thiazol-4-yl)-7-methoxy-quinolin-4-yloxy]-5-[(1R,2S)-1-carbonylamino-2-vinyl-cyclopropyl]-2,2-dioxo-2λ*6*-thia-3,6,12,22-tetraaza-tricyclo[21.4.0.0*8,12*]heptacosa-1(27),23,25-triene-4,7,13-trione

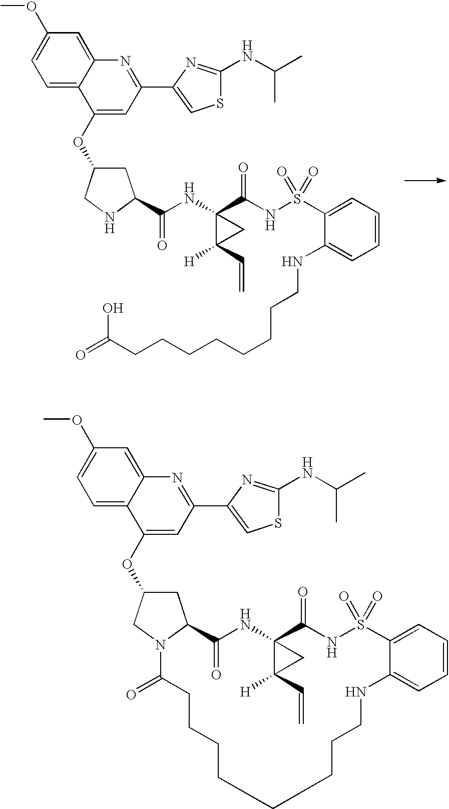

The title compound is prepared analogously as described for the title compound in Example 2 using 168 mg (0.14 mmol) 9-(2-{[(1R,2S)-1-({(2S,4R)-4-[2-(2-Isopropylamino-thiazol-4-yl)-7-methoxy-quinolin-4-yloxy]-pyrrolidine-2-carbonyl}-amino)-2-vinyl-cyclopropanecarbonyl]-sulfamoyl}-phenylamino)-nonanoic acid (TFA-salt), 182 mg (1.4 mmol) DIPEA and 268 mg (0.71 mmol) HATU in 75 mL DCM and 1 mL DMF.

HPLC (method A) t_{R}=5.90 min

TLC, Rf (CH₂Cl₂/MeOH 19:1)=0.37

MS (method D): 830 [M+]

### Preparation of 9-(2-{[(1R,2S)-1-({(2S,4R)-4-[2-(2-Isopropylamino-thiazol-4-yl)-7-methoxy-quinolin-4-yloxy]-pyrrolidine-2-carbonyl}-amino)-2-vinyl-cyclopropanecarbonyl]-sulfamoyl}-phenylamino)-nonanoic acid

### Step 1

### (2S,4R)-4-[2-(2-Isopropylamino-thiazol-4-yl)-7-methoxy-quinolin-4-yloxy]-2-{(1R,2S)-1-[2-(8-methoxycarbonyl-octylamino)-benzenesulfonylaminocarbonyl]-2-vinyl-cyclopropylcarbamoyl}-pyrrolidine-1-carboxylic acid tert-butyl ester

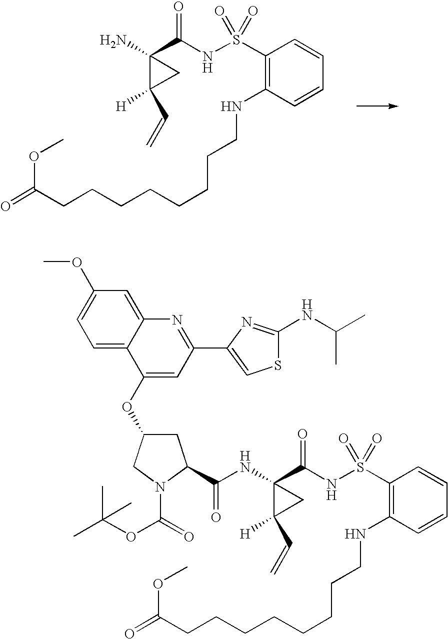

The title compound is prepared analogously as described for the title compound in Example 2 (step 1) using 200 mg (0.44 mmol) 9-{2-[((1R,2S)-1-Amino-2-vinyl-cyclopropanecarbonyl)-sulfamoyl]-phenyl-amino}-nonanoic acid methyl ester, 234 mg (0.44 mmol) (2S,4R)-4-[2-(2-Isopropylamino-thiazol-4-yl)-7-methoxy-quinolin-4-yloxy]-pyrrolidine-1,2-dicarboxylic acid 1-tert-butyl ester, 219 mg (0.58 mmol) HATU and 287 mg (2.2 mmol) DIPEA in 5 mL DMF.

HPLC (method A) t_{R}=6.1 min

TLC, Rf (CH₂Cl₂/MeOH 9:1)=0.81

MS (method D): 962 [M+]

### Step 2

### (2S,4R)-2-{(1R,2S)-1-[2-(8-Carboxy-octylamino)-benzenesulfonylaminocarbonyl]-2-vinyl-cyclopropylcarbamoyl}-4-[2-(2-isopropylamino-thiazol-4-yl)-7-methoxy-quinolin-4-yloxy[-pyrrolidine-1-carboxylic acid tert-butyl ester

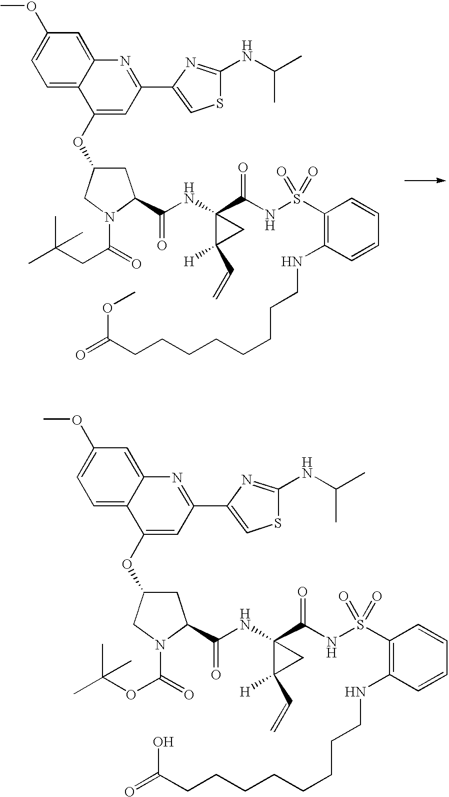

The title compound is prepared analogously as described for the title compound in Example 2 (step 2) using 174 mg (0.18 mmol) (2S,4R)-4-[2-(2-Isopropylamino-thiazol-4-yl)-7-methoxy-quinolin-4-yloxy]-2-{(1R,2S)-1-[2-(8-methoxycarbonyl-octylamino)-benzenesulfonylaminocarbonyl]-2-vinyl-cyclopropyl-carbamoyl}-pyrrolidine-1-carboxylic acid tert-butyl ester and 44 mg (1.81 mmol) LiOH in 14 mL THF/MeOH/H₂O (2:1:1).

HPLC (method A) t_{R}=5.58 min

TLC, Rf (CH₂Cl₂/MeOH)=0.27

MS (method D): 948 [M+]

### Step 3

### 9-(2-{[(1R,2S)-1-({(2S,4R)-4-[2-(2-Isopropylamino-thiazol-4-yl)-7-methoxy-quinolin-4-yloxy]-pyrrolidine-2-carbonyl)-amino)-2-vinyl-cyclopropanecarbonyl}-sulfamoyl}-phenylamino)-nonanoic acid

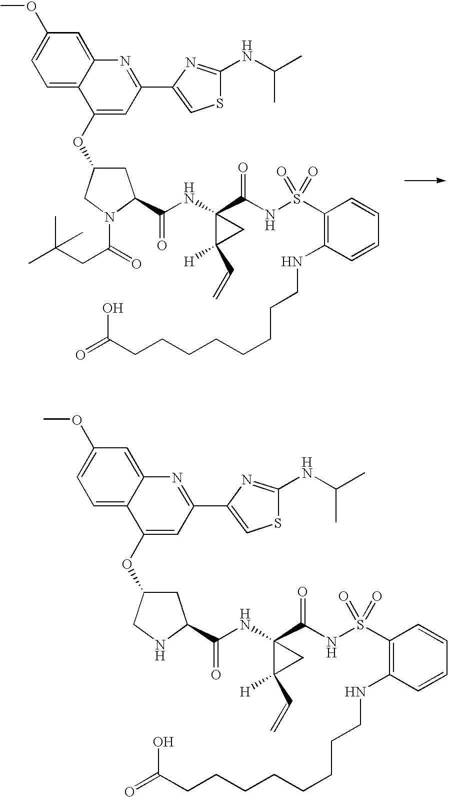

The title compound is prepared analogously as described for the title compound in Example 2 (step 3) using 135 mg (0.14 mmol) (2S,4R)-2-{(1R,2S)-142-(8-Carboxy-octylamino)-benzenesulfonylamino-carbonyl]-2-vinyl-cyclopropylcarbamoyl}-4-[2-(2-isopropylamino-thiazol-4-yl)-7-methoxy-quinolin-4-yloxy]-pyrrolidine-1-carboxylic acid tert-butyl ester and 0.6 mL TFA in 10 mL DCM.

HPLC (method A) t_{R}=5.20 min

TLC, Rf (CH₂Cl₂/MeOH/H₂O/AcOH 90:10:1:0.5)=0.19

MS (method D): 848 [M+]

### Example 13

### 4-Fluoro-1,3-dihydro-isoindole-2-carboxylic acid (8S,10R)-5-[(1R,2S)-1-carbonylamino-2-vinyl-cyclopropyl]-2,2,4,7,13-pentaoxo-2λ*6*-thia-3,6,12,22-tetraaza-tricyclo[21.4.0.0*8,12*]heptacosa-1(27),23,25-trien-10-yl ester

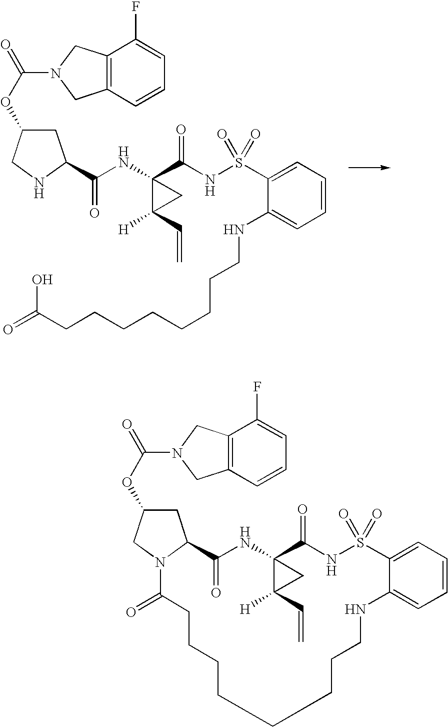

The title compound is prepared analogously as described for the title compound in Example 2 using 78 mg (0.08 mmol) 4-Fluoro-1,3-dihydro-isoindole-2-carboxylic acid (3R,5S)-5-{(1R,2S)-1-[2-(8-carboxy-octylamino)-benzenesulfonylaminocarbonyl]-2-vinyl-cyclopropylcarbamoyl}-pyrrolidin-3-yl ester (TFA-salt), 107 mg (0.83 mmol) DIPEA and 158 mg (0.42 mmol) HATU in 50 mL DCM and 1 mL DMF.

HPLC (method A) t_{R}=5.65 min

TLC, Rf (CH₂Cl₂/MeOH 19:1)=0.27

MS (method D): 696 [M+]

### Preparation of 4-Fluoro-1,3-dihydro-isoindole-2-carboxylic acid (3R,5S)-5-{(1R,2S)-1-[2-(8-carboxy-octylamino)-benzenesulfonylaminocarbonyl]-2-vinyl-cyclopropylcarbamoyl}-pyrrolidin-3-yl ester

### Step 1

### 4-Fluoro-1,3-dihydro-isoindole-2-carboxylic acid (3R,5S)-1-tert-butoxycarbonyl-5-{(1R,2S)-1-[2-(8-methoxycarbonyl-octylamino)-benzenesulfonylaminocarbonyl]-2-vinyl-cyclopropylcarbamoyl}-pyrrolidin-3-yl ester

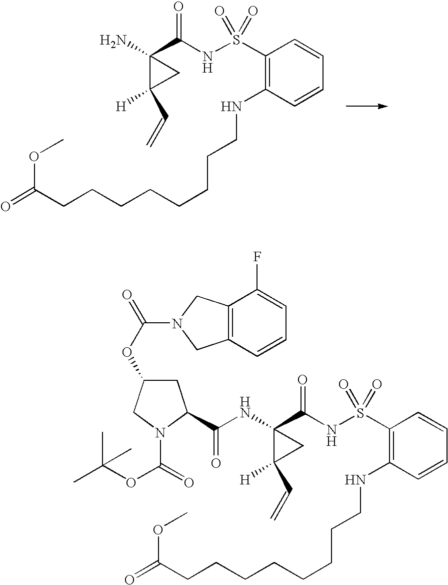

The title compound is prepared analogously as described for the title compound in Example 2 (step 1) using 150 mg (0.18 mmol) 9-{2-[((1R,2S)-1-Amino-2-vinyl-cyclopropanecarbonyl)-sulfamoyl]-phenyl-amino}-nonanoic acid methyl ester, 71 mg (0.18 mmol) (2S,4R)-4-(4-Fluoro-1,3-dihydro-isoindole-2-carbonyloxy)-pyrrolidine-1,2-dicarboxylic acid 1-tert-butyl ester, 103 mg (0.27 mmol) HATU and 70 mg (0.54 mmol) DIPEA in 5 mL DCM.

HPLC (method A) t_{R}=6.10 min

TLC, Rf (CH₂Cl₂/MeOH 9:1)=0.69

MS (method D): 828 [M+]

### Step 2

### 4-Fluoro-1,3-dihydro-isoindole-2-carboxylic acid (3R,5S)-1-tert-butoxycarbonyl-5-{(1R,2S)-1-[2-(8-carboxy-octylamino)-benzenesulfonylaminocarbonyl]-2-vinyl-cyclopropylcarbamoyl}-pyrrolidin-3-yl ester

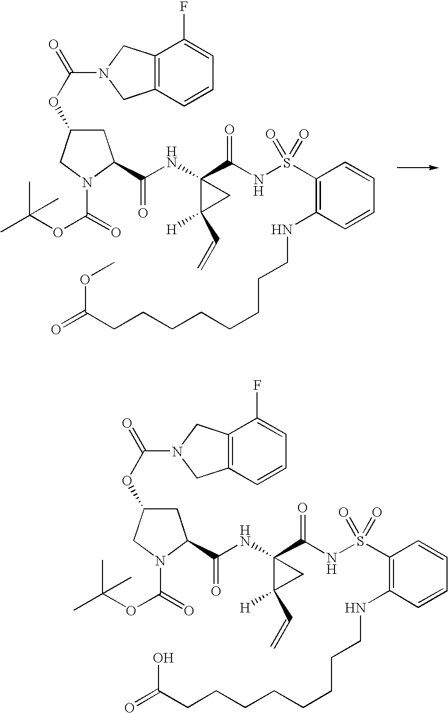

The title compound is prepared analogously as described for the title compound in Example 2 (step 2) using 80 mg (0.09 mmol) 4-Fluoro-1,3-dihydro-isoindole-2-carboxylic acid (3R,5S)-1-tert-butoxycarbonyl-5-{(1R,2S)-1-[2-(8-methoxycarbonyl-octylamino)-benzenesulfonylaminocarbonyl]-2-vinyl-cyclopropylcarbamoyl}-pyrrolidin-3-yl ester and 36 mg (0.85 mmol) LiOH in 12 mL THF/MeOH/H₂O (2:1:1).

HPLC (method A) t_{R}=5.53 min

TLC, Rf (CH₂Cl₂/MeOH 9:1)=0.51

MS (method D): 814 [M+]

### Step 3

### 4-Fluoro-1,3-dihydro-isoindole-2-carboxylic acid (3R,5S)-5-{(1R,2S)-1-[2-(8-carboxy-octylamino)-benzenesulfonylaminocarbonyl]-2-vinyl-cyclopropylcarbamoyl}-pyrrolidin-3-yl ester

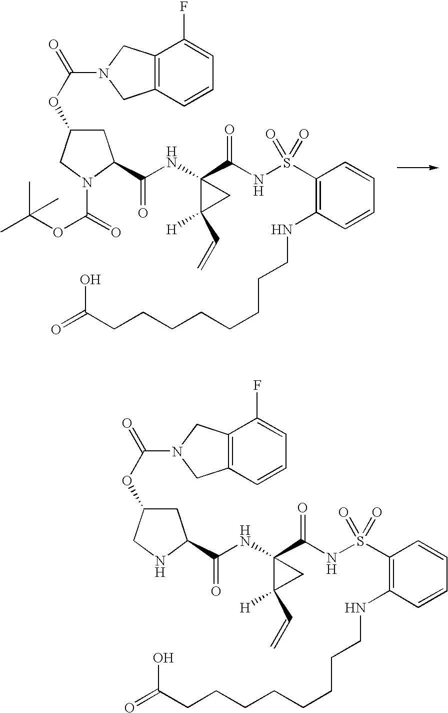

The title compound is prepared analogously as described for the title compound in Example 2 (step 3) using 68 mg (0.08 mmol) 4-Fluoro-1,3-dihydro-isoindole-2-carboxylic acid (3R,5S)-1-tert-butoxycarbonyl-5-{(1R,2S)-1-[2-(8-carboxy-octylamino)-benzenesulfonylaminocarbonyl]-2-vinyl-cyclopropylcarbamoyl}-pyrrolidin-3-yl ester and 1 mL TFA in 5 mL DCM.

HPLC (method A) t_{R}=4.74 min

TLC, Rf (CH₂Cl₂/MeOH 9:1)=0.35

MS (method D): 714 [M+]

### Example 14

### (1R,2S,2′R,25a′S)-2′-[(7-methoxy-2-phenylquinolin-4-yl)oxy]-2-vinyl-1′H,2′H,3′H,5′H,6′H,7′H,13′H,14′H,15′H,21′H,22′H,24′H,25′H,25a′H-spiro[cyclopropane-1,23′-[20]thia[4,15,21,241tetraaza[8,12](metheno)pyrrolo[2,1-g][1,2,5,8,19]benzothiatetraazacyclohenicosine]-5′,14′,22′,25′-tetrone 20′,20′-dioxide

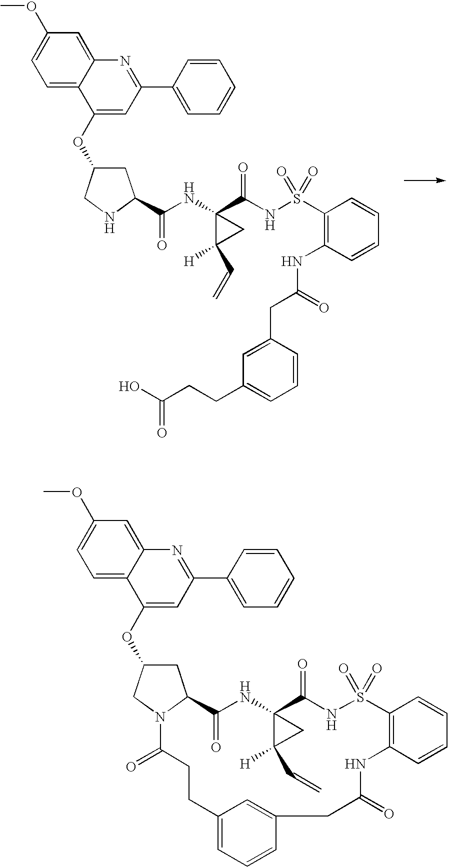

The title compound is prepared analogously as described for the title compound in Example 2 using 23 mg (0.03 mmol) 3-[3-({2-R(1R,2S)-1-{[(2S,4R)-4-(7-Methoxy-2-phenyl-quinolin-4-yloxy)-pyrrolidine-2-carbonyl]-amino}-2-vinyl-cyclopropanecarbonyl)-sulfamoyl]-phenylcarbamoyl}-methyl)-phenyl]-propionic acid (TFA-salt), 32 mg (0.25 mmol) DIPEA and 48 mg (0.71 mmol) HATU in 10 mL DCM and 0.2 mL DMF.

HPLC (method A) t_{R}=4.58 min

TLC, Rf (CH₂Cl₂/MeOH 9:1)=0.44

MS (method D): 800 [M+]

### Preparation of 3-[3-({2-[((1R,2S)-1-{[(2S,4R)-4-(7-Methoxy-2-phenyl-quinolin-4-yloxy)-pyrrolidine-2-carbonyl]-amino}-2-vinyl-cyclopropanecarbonyl)-sulfamoyl]-phenylcarbamoyl}-methyl)-phenyl]-propionic acid

### Step 1

### (E)-3-(3-Carboxymethyl-phenyl)-acrylic acid methyl ester

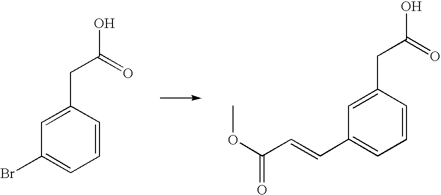

A microwave-vial is charged with 2.2 g (10 mmol) 3-Bromophenylacetic acid, 2.62 g (30 mmol) Methyl acrylate, 0.31 g (1.0 mmol) P(o-tol)₃, 90 mg (0.4 mmol) Pd(OAc)₂, amd 1.2 g (12 mmol) NEt₃. The vial is purged with argon, sealed and heated in the microwave (Personal Chemistry, Emrys Optimizer) for 15 min at 150° C. After cooling to rt the mixture is diluted with water and EtOAc, filtered through a pad of Hyflo and washed thoroughly with EtOAc. The filtrate is separated, the aqueous phase is extracted with EtOAc and the combined organic phases are dried with Na₂SO₄, filtered and the solvent is removed in vacuo. The residue is purified by FC on silica (eluent: CH₂Cl₂/MeOH 98:2→95:5) to give the title compound as a colorless solid.

HPLC (method A) t_{R}=3.14 min

TLC, Rf (CH₂Cl₂/MeOH 19:1)=0.22

MS (method D): 221 [M+H]

### Step 2

### 3-(3-Carboxymethyl-phenyl)-propionic acid methyl ester

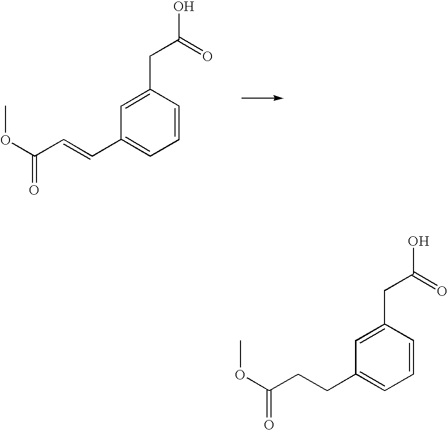

A shaking flask charged with 3.9 g (16.0 mmol) (E)-3-(3-Carboxymethyl-phenyl)-acrylic acid methyl ester and 0.4 g 10% Pd/C (Engelhard 4505) in 80 mL EtOAc is purged with hydrogen and shaken for 10 h. The catalyst is removed by filtration, washed with EtOAc and the filtrate is concentrated in vacuo to give the title compound as a colorless solid which is used without further purification.

HPLC (method A) t_{R}=2.96 min

TLC, Rf (CH₂Cl₂/MeOH 19:1)=0.19

MS (method D): 240 [M+H₂O]

### Step 3

### 3-[3-({2-[((1R,2S)-1-tert-Butoxycarbonylamino-2-vinyl-cyclopropanecarbonyl)-sulfamoyl]-phenylcarbamoyl}-methyl)-phenyl]-propionic acid methyl ester

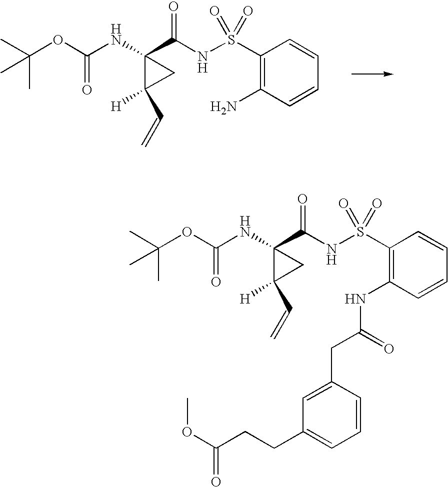

The title compound is prepared analogously as described for the title compound in Example 1 (Step 2) using 1.0 g (2.6 mmol) [(1R,2S)-1-(2-Amino-benzenesulfonylaminocarbonyl)-2-vinyl-cyclopropyl]-carbamic acid tert-butyl ester, 2.0 g (9.0 mmol) 3-(3-Carboxymethyl-phenyl)-propionic acid methyl ester, 1.30 g (10.8 mmol) Benzotriazole, 1.30 g (10.8 mmol) Thionylchloride, 2.65 g (26 mmol) NEt₃ and 100 mg DMAP in 40 mL DCM.

HPLC (method A) t_{R}=4.90 min

TLC, Rf (CH₂Cl₂/MeOH 19:1)=0.36

MS (method D): 613 [M+H₂O]

Step 4

### 3-[3-({2-[((1R,2S)-1-Amino-2-vinyl-cyclopropanecarbonyl)-sulfamoyl]-phenylcarbamoyl}-methyl)-phenyl]-propionic acid methyl ester

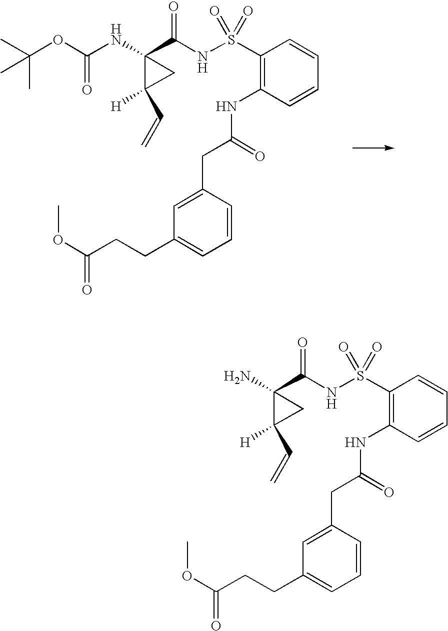

The title compound is prepared analogously as described for the title compound in Example 1 (Step 3) using 0.38 g (0.59 mmol) 3-[3-({2-[((1R,2S)-1-tert-Butoxycarbonylamino-2-vinyl-cyclopropane-carbonyl)-sulfamoyl]-phenylcarbamoyl}-methyl)-phenyl]-propionic acid methyl ester and 5 mL 4N HCl in Dioxane.

HPLC (method A) t_{R}=3.09 min

MS (method D): 486 [M+]

### Step 5

### (2S,4R)-2-[(1R,2S)-1-(2-{2-[3-(2-Methoxycarbonyl-ethyl)-phenyl]-acetylamino}-benzenesulfonylaminocarbonyl)-2-vinyl-cyclopropylcarbamoyl]-4-(7-methoxy-2-phenyl-quinolin-4-yloxy)-pyrrolidine-1-carboxylic acid tert-butyl ester

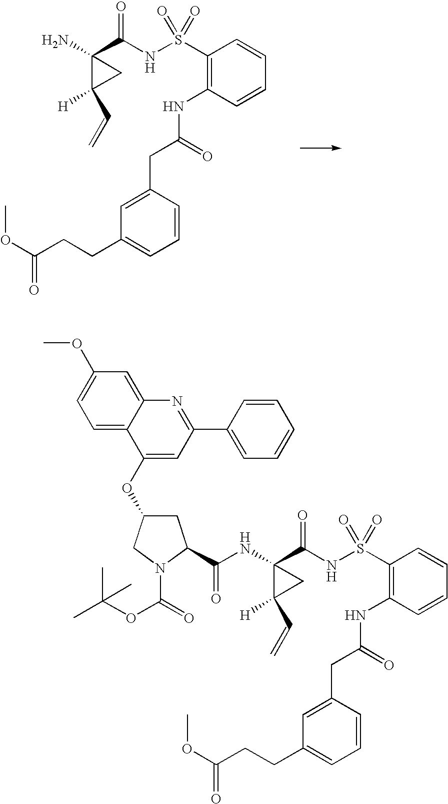

The title compound is prepared analogously as described for the title compound in Example 1 (step 4) using 114 mg (0.59 mmol) 3-[3-({2-[((1R,2S)-1-Amino-2-vinyl-cyclopropanecarbonyl)-sulfamoyl]-phenylcarbamoyl}-methyl)-phenyl]-propionic acid methyl ester, 73 mg (0.16 mmol) (2S,4R)-4-(7-Methoxy-2-phenyl-quinolin-4-yloxy)-pyrrolidine-1,2-dicarboxylic acid 1-tert-butyl esterr, 90 mg (0.24 mmol) HATU and 102 mg (0.79 mmol) DIPEA in 5 mL DMF.

HPLC (method A) t_{R}=5.20 min

TLC, Rf (CH₂Cl₂/MeOH 9:1)=0.38

MS (method D): 932 [M+]

### Step 6

### (2S,4R)-2-[(1R,2S)-1-(2-{2-[3-(2-Methoxycarbonyl-ethyl)-phenyl]-acetylamino}-benzenesulfonylaminocarbonyl)-2-vinyl-cyclopropylcarbamoyl]-4-(7-methoxy-2-phenyl-quinolin-4-yloxy)-pyrrolidine-1-carboxylic acid tert-butyl ester

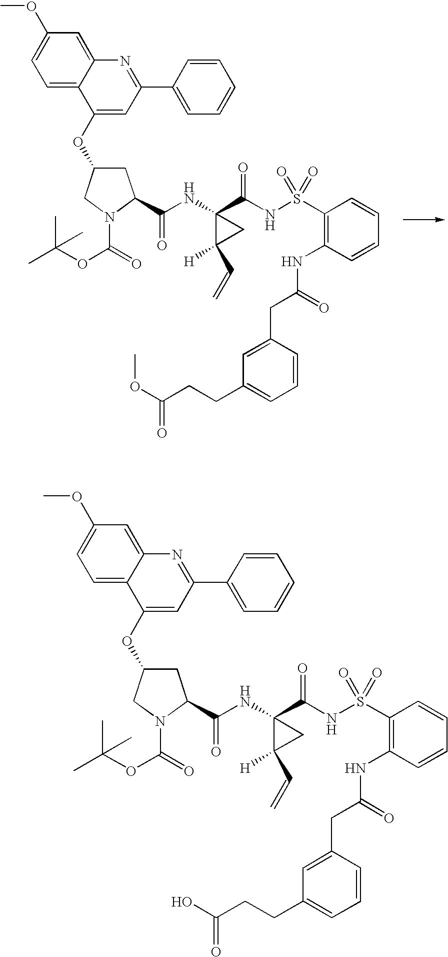

The title compound is prepared analogously as described for the title compound in Example 2 (step 2) using 28 mg (0.03 mmol) (2S,4R)-2-[(1R,2S)-1-(2-{2-[3-(2-Methoxycarbonyl-ethyl)-phenyl]-acetyl-amino}-benzenesulfonylaminocarbonyl)-2-vinyl-cyclopropylcarbamoyl]-4-(7-methoxy-2-phenyl-quinolin-4-yloxy)-pyrrolidine-1-carboxylic acid tert-butyl ester and 13 mg (0.3 mmol) LiOH in 8 mL THF/MeOH/H₂O (2:1:1).

HPLC (method A) t_{R}=4.77 min

TLC, Rf (CH₂Cl₂/MeOH 9:1)=0.17

MS (method D): 918 [M+]

### Step 7

### 3-[3-({2-[((1R,2S)-1-{[(2S,4R)-4-(7-Methoxy-2-phenyl-quinolin-4-yloxy)-pyrrolidine-2-carbonyl]-amino}-2-vinyl-cyclopropanecarbonyl)-sulfamoyl]-phenylcarbamoyl}-methyl)-phenyl]-propionic acid

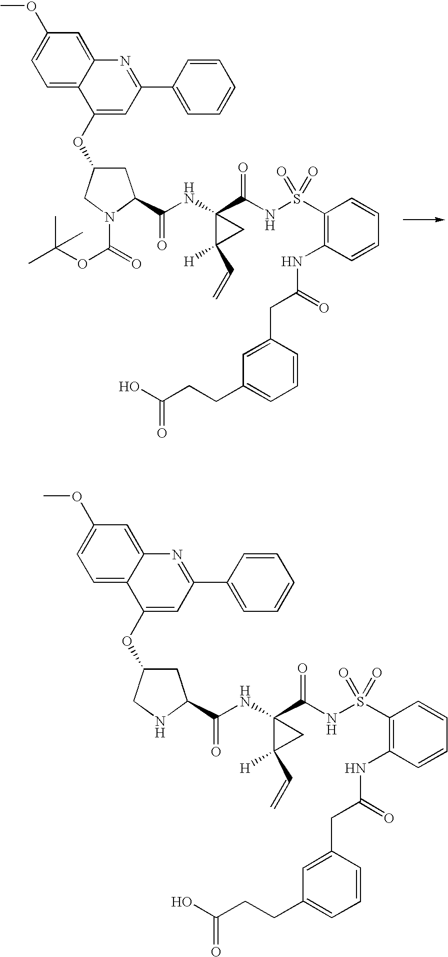

The title compound is prepared analogously as described for the title compound in Example 2 (step 3) using 26 mg (0.03 mmol) (2S,4R)-2-[(1R,2S)-1-(2-{2-[3-(2-Carboxy-ethyl)-phenyl]-acetylamino}-benzenesulfonylaminocarbonyl)-2-vinyl-cyclopropylcarbamoyl]-4-(7-methoxy-2-phenyl-quinolin-4-yloxy)-pyrrolidine-1-carboxylic acid tert-butyl ester and 1 mL

TFA in 5 mL DCM.

HPLC (method A) t_{R}=3.82 min

TLC, Rf (CH₂Cl₂/MeOH 9:1)=0.35

MS (method D): 818 [M+]

### Example 15

### 8S,10R)-10-[2-(2-Isopropylamino-thiazol-4-yl)-7-methoxy-quinolin-4-yloxy]-5-[(1R,2S)-1-carbonylamino-2-vinyl-cyclopropyl]-2,2-dioxo-16,19-dioxa-2λ*6*-thia-3,6,12,22-tetraaza-tricyclo[21.4.0.0*8,12*]heptacosa-1(27),23,25-triene-4,7,13,21-tetraone

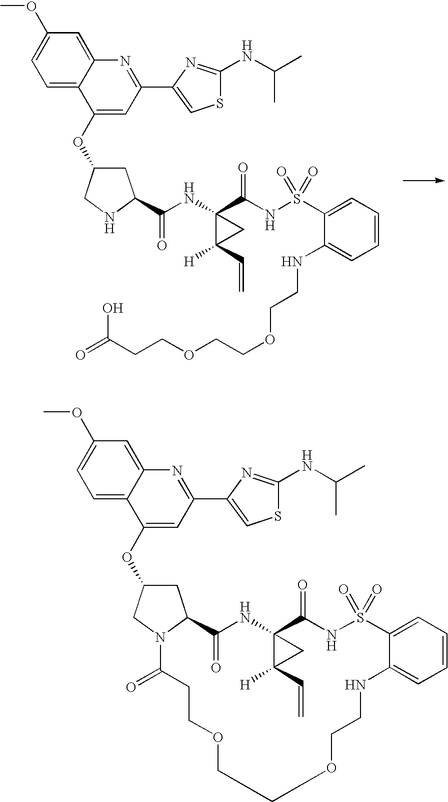

The title compound is prepared analogously as described for the title compound in Example 2 using 20 mg (0.02 mmol) 3-{2-[2-(2-{[(1R,2S)-1-({(2S,4R)-4-[2-(2-Isopropylamino-thiazol-4-yl)-7-methoxy-quinolin-4-yloxy]-pyrrolidine-2-carbonyl}-amino)-2-vinyl-cyclopropanecarbonyl]-sulfamoyl}-phenylaminoyethoxy]-ethoxy}-propionic acid (TFA-salt), 22 mg (0.20 mmol) DIPEA and 32 mg (0.09 mmol) HATU in 10 mL DCM and 0.2 mL DMF.

HPLC (method A) t_{R}=4.65 min

TLC, Rf (CH₂Cl₂/MeOH 9:1)=0.34

MS (method D): 834 [M+]

### Preparation of 3-{2-[2-(2-{[(1R,2S)-1-({(2S,4R)-4-[2-(2-Isopropylamino-thiazol-4-yl)-7-methoxy-quinolin-4-yloxy]-pyrrolidine-2-carbonyl}-amino)-2-vinyl-cyclopropanecarbonyl]-sulfamoyl}-phenylamino)-ethoxy]-ethoxy}-propionic acid

### Step 1

### 3-(2-Allyloxy-ethoxy)-propionic acid methyl ester

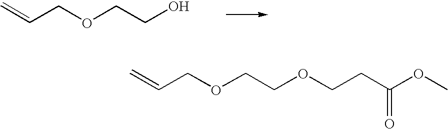

To a solution of 20 g (0.19 mol) 2-Allyloxyethanol in 250 mL abs. THF is added 44 mg Sodium and the mixture is refluxed until the sodium disappears. After cooling to RT 28.3 g (0.33 mol) methyl acrylate is added and stirring is continued overnight. The solvent is removed in vacuo, 400 mL MeOH and 1 mL conc. H₂SO₄ is added and the mixture is refluxed overnight. The solvent is removed in vacuo and the residue is purified by FC on silica (eluent: hexane/EtOAc 3:1) to give the title compound as a colorless oil.

TLC, Rf (hexane/EtOAc 3:1)=0.48

MS (method D): 206 [M+18]

### Step 2

### 3-[2-(2-Oxo-ethoxy)-ethoxy]-propionic acid methyl ester

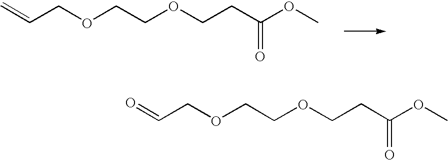

A suspension of 1.5 g (8.0 mmol) 3-(2-Allyloxy-ethoxy)-propionic acid methyl ester and 134 mg (1.6 mmol) sodium bicarbonate in 160 mL DCM is cooled to −78° C. Ozone is bubbled through until a blue color appears (˜15 min). Oxygen is bubbled through the mixture for 2 min to remove excess of ozone, 2.7 g (10 mmol) PPh₃ is added and stirring is continued for 1 h at −78° C. After warming to RT, the solvent is removed in vacuo and the residue is used without further purification.

### Step 3

### 3-[2-(2-{2-[((1R,2S)-1-tert-Butoxycarbonylamino-2-vinyl-cyclopropanecarbonyl)-sulfamoyl]-phenylamino}-ethoxy)-ethoxy]-propionic acid methyl ester

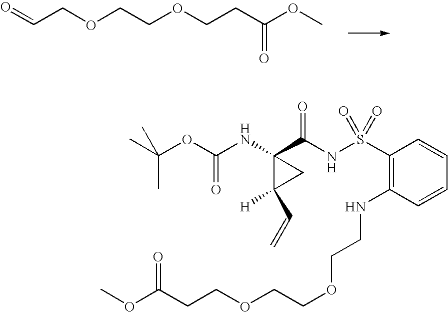

The title compound is prepared analogously as described for the title compound in Example 11 (step 3) using 200 mg (0.52 mmol) [(1R,2S)-1-(2-Amino-benzenesulfonylaminocarbonyl)-2-vinyl-cyclopropyl]-carbamic acid tert-butyl ester, 500 mg crude 3-[2-(2-Oxo-ethoxy)-ethoxy]-propionic acid methyl ester (from the previous step), 292 mg (1.31 mmol) NaBH(OAc)₃ and 94 mg (1.6 mmol) AcOH in 20 mL 1,2 DCE

HPLC (method A) t_{R}=4.57 min

MS (method D): 556 [M+]

### Step 4

### 3-[2-(2-{2-[((1R,2S)-1-Amino-2-vinyl-cyclopropanecarbonyl)-sulfamoyl]-phenylamino}-ethoxy)-ethoxy]-propionic acid methyl ester

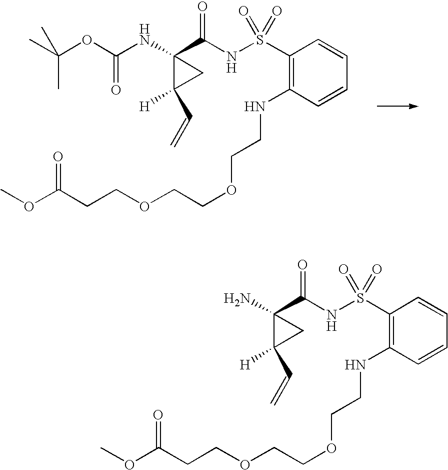

The title compound is prepared analogously as described for the title compound in Example 11 (step 4) using 485 mg (0.58 mmol) 3-[2-(2-{2-[((1R,2S)-1-tert-Butoxycarbonylamino-2-vinyl-cyclopropanecarbonyl)-sulfamoyl]-phenylamino}-ethoxy)-ethoxy}-propionic acid methyl ester and 1.5 mL TFA in 20 mL DCM.

HPLC (method A) t_{R}=2.64 min

MS (method D): 456 [M+]

### Step 5

### (2S,4R)-4-[2-(2-Isopropylamino-thiazol-4-yl)-7-methoxy-quinolin-4-yloxy]-2-[(1R,2S)-1-(2-{2-[2-(2-methoxycarbonyl-ethoxy)-ethoxy]-ethylamino}-benzenesulfonylaminocarbonyl)-2-vinyl-cyclopropylcarbamoyl]-pyrrolidine-1-carboxylic acid tert-butyl ester

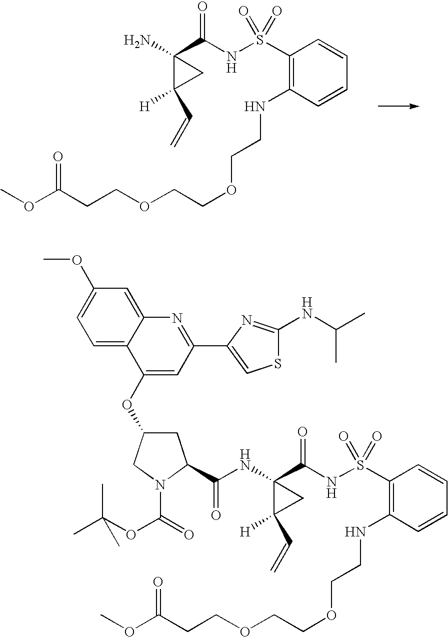

The title compound is prepared analogously as described for the title compound in Example 2 (step 1) using 235 mg (0.34 mmol) 3-[2-(2-{2-[((1R,2S)-1-Amino-2-vinyl-cyclopropanecarbonyl)-sulfamoyl]-phenylamino}-ethoxy)-ethoxy]-propionic acid methyl ester, 182 mg (0.34 mmol) (2S,4R)-4-[2-(2-Isopropylamino-thiazol-4-yl)-7-methoxy-quinolin-4-yloxy]-pyrrolidine-1,2-dicarboxylic acid 1-tert-butyl ester, 196 mg (0.52 mmol) HATU and 134 mg (1.0 mmol) DIPEA in 20 mL DCM.

HPLC (method A) t_{R}=5.08 min

TLC, Rf (CH₂Cl₂/MeOH 9:1)=0.31

MS (method D): 966 [M+]

### Step 6

### (2S,4R)-2-[(1R,2S)-1-(2-{2-[2-(2-Carboxy-ethoxy)-ethoxy]-ethylamino}-benzenesulfonylamino-carbonyl)-2-vinyl-cyclopropylcarbamoyl]-4-[2-(2-isopropylamino-thiazol-4-yl)-7-methoxy-quinolin-4-yloxy]-pyrrolidine-1-carboxylic acid tert-butyl ester

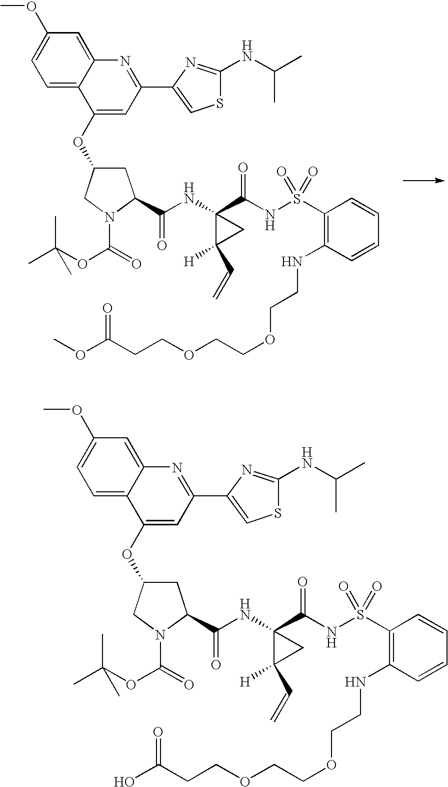

The title compound is prepared analogously as described for the title compound in Example 2 (step 2) using 170 mg (0.18 mmol) (2S,4R)-4-[2-(2-Isopropylamino-thiazol-4-yl)-7-methoxy-quinolin-4-yloxy]-2-[(1R,2S)-1-(2-{2-[2-(2-methoxycarbonyl-ethoxy)-ethoxy]-ethylamino}-benzenesulfonylaminocarbonyl)-2-vinyl-cyclopropylcarbamoyl]-pyrrolidine-1-carboxylic acid tert-butyl ester (TFA-salt) and 76 mg (1.8 mmol) LiOH in 20 mL THF/MeOH/H₂O (2:1:1).

HPLC (method A) t_{R}=4.79 min

TLC, Rf (CH₂Cl₂/MeOH 9:1)=0.33

MS (method D): 952 [M+]

### Step 7

### (2S,4R)-2-[(1R,2S)-1-(2-{2-[2-(2-Carboxy-ethoxy)-ethoxy]-ethylamino}-benzenesulfonylamino-carbonyl)-2-vinyl-cyclopropylcarbamoyl]-4-[2-(2-isopropylamino-thiazol-4-yl)-7-methoxy-quinolin-4-yloxy]-pyrrolidine-1-carboxylic acid tert-butyl ester

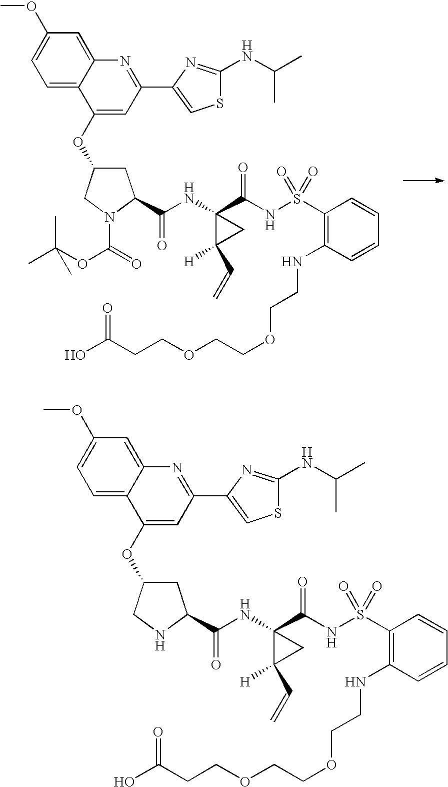

The title compound is prepared analogously as described for the title compound in Example 2 (step 3) using 12 mg (0.01 mmol) (2S,4R)-2-[(1R,2S)-1-(2-{2-[2-(2-Carboxy-ethoxy)-ethoxy]-ethylamino}-benzenesulfonylamino-carbonyl)-2-vinyl-cyclopropylcarbamoyl]-4-[2-(2-isopropylamino-thiazol-4-yl)-7-methoxy-quinolin-4-yloxy]-pyrrolidine-1-carboxylic acid tert-butyl ester (TFA-salt) and 0.1 mL TFA in 3 mL DCM.

HPLC (method A) t_{R}=4.27 min

TLC, Rf (CH₂Cl₂/MeOH/H₂O/AcOH 90:10:1:0.5)=0.26

MS (method D): 852 [M+]

### Example 16

### (8S,10R)-10-[2-(2-Isopropylamino-thiazol-4-yl)-7-methoxy-quinolin-4-yloxy]-5-[(1R,2S)-1-carbonylamino-2-vinyl-cyclopropyl]-2,2-dioxo-16,19-dioxa-2λ*6*-thia-3,6,12,22-tetraaza-tricyclo[21.4.0.0*8,12*]heptacosa-1(27),23,25-triene-4,7,13,21-tetraone

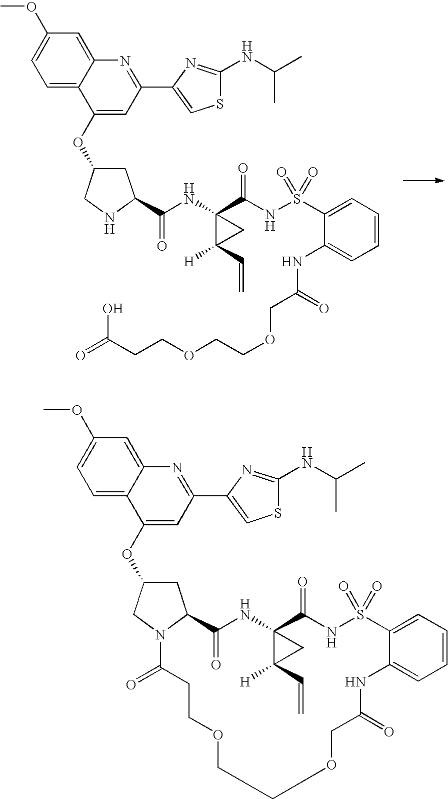

The title compound is prepared analogously as described for the title compound in Example 2 using 108 mg (0.10 mmol) 3-{2-[(2-{[(1R,2S)-1-({(2S,4R)-4-[2-(2-Isopropylamino-thiazol-4-yl)-7-methoxy-quinolin-4-yloxy]-pyrrolidine-2-carbonyl}-amino)-2-vinyl-cyclopropanecarbonyl]-sulfamoyl)-phenylcarbamoyl)-methoxy]-ethoxy}-propionic acid (TFA-salt), 128 mg (1.0 mmol) DIPEA and 188 mg (0.5 mmol) HATU in 100 mL DCM and 2 mL DMF.

HPLC (method A) t_{R}=4.50 min

TLC, Rf (CH₂Cl₂/MeOH 9:1)=0.18

MS (method D): 848 [M+]

### Preparation of 33-{2-[(2-{[(1R,2S)-1-({(2S,4R)-4-[2-(2-Isopropylamino-thiazol-4-yl)-7-methoxy-quinolin-4-yloxy]-pyrrolidine-2-carbonyl}-amino)-2-vinyl-cyclopropanecarbonyl]-sulfamoyl}-phenylcarbamoyl)-methoxyl-ethoxy}-propionic acid

### Step 1

### 3-(2-Carboxymethoxy-ethoxy)-propionic acid methyl ester

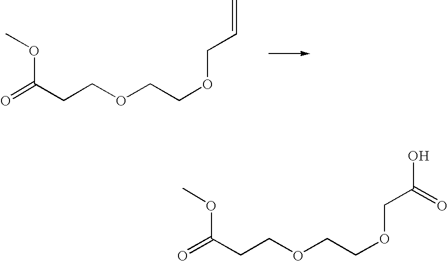

To a solution of 1.0 g (5.3 mmol) 3-(2-Allyloxy-ethoxy)-propionic acid methyl ester (according to example 15 step 1) in 50 mL CCl₄/ACN/H₂O (2:2:3) is added 5.68 g (27 mmol Sodium(meta)periodate followed by 135 mg (0.27 mmol) RuCl₃ monohydrate at RT. After stirring overnight the reaction is diluted with water and extracted thoroughly with DCM and the organic phase is discarded. The aq. phase is adjusted to pH 1 by addition of 4N HCl, and extracted thoroughly (12×) with DCM. The organic phase is dried with Na₂SO₄, filtered and the solvent is removed in vacuo. The residue is used without further purification.

TLC, Rf (CH₂Cl₂/MeOH 9:1)=0.16

MS (method D): 224 [M+18]

### Step 2

### 3-[2-({2-[((1R,2S)-1-tert-Butoxycarbonylamino-2-vinyl-cyclopropanecarbonyl)-sulfamoyl]-phenylcarbamoyl}-methoxy)-ethoxy]-propionic acid methyl ester

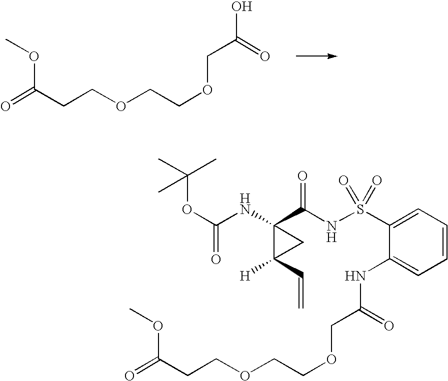

The title compound is prepared analogously as described for the title compound in Example 1 (Step 2) using 100 mg (0.26 mmol) [(1R,2S)-1-(2-Amino-benzenesulfonylaminocarbonyl)-2-vinyl-cyclopropyl]-carbamic acid tert-butyl ester, 200 mg (0.97 mmol) 3-(2-Carboxymethoxy-ethoxy)-propionic acid methyl ester, 140 mg (1.2 mmol) Benzotriazole, 140 mg (1.2 mmol) Thionylchloride, 265 mg (2.6 mmol) NEt₃ and 20 mg DMAP in 20 mL DCM.

HPLC (method A) t_{R}=4.31 min

TLC, Rf (CH₂Cl₂/MeOH 19:1)=0.56

MS (method D): 570 [M+]

### Step 3

### 3-[2-({2-[((1R,2S)-1-Amino-2-vinyl-cyclopropanecarbonyl)-sulfamoyl]-phenylcarbamoyl}-methoxy)-ethoxyl-propionic acid methyl ester

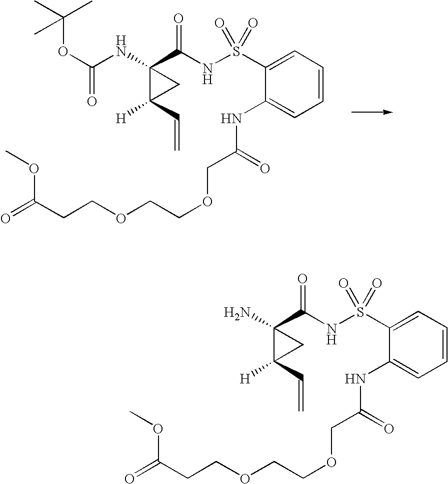

The title compound is prepared analogously as described for the title compound in Example 11 (step 4) using 116 mg (0.20 mmol) 33-[2-({2-[((1R,2S)-1-tert-Butoxycarbonylamino-2-vinyl-cyclopropanecarbonyl)-sulfamoyl]-phenylcarbamoyl}-methoxy)-ethoxy]-propionic acid methyl ester and 0.5 mL TFA in 6 mL DCM.

HPLC (method A) t_{R}=1.95 min

TLC, Rf (CH₂Cl₂/MeOH 19:1)=0.32

MS (method D): 470 [M+]

### Step 4

### (2S,4R)-4-[2-(2-Isopropylamino-thiazol-4-yl)-7-methoxy-quinolin-4-yloxy]-2-[(1R,2S)-1-(2-{2-[2-(2-methoxycarbonyl-ethoxy)-ethoxyl-acetylamino}-benzenesulfonylaminocarbonyl)-2-vinyl-cyclopropylcarbamoyl]-pyrrolidine-1-carboxylic acid tert-butyl ester

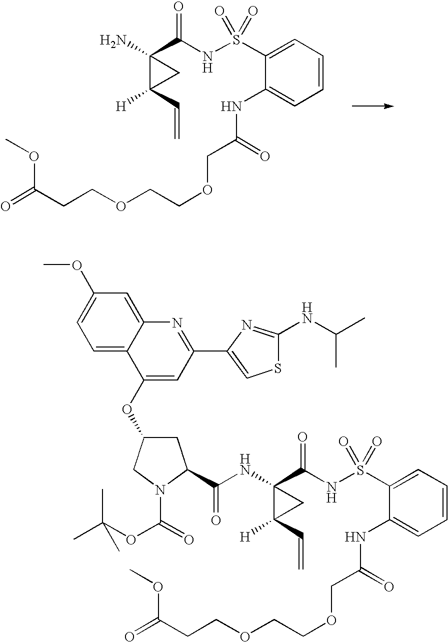

The title compound is prepared analogously as described for the title compound in Example 2 (step 1) using 118 mg (0.20 mmol) 33-[2-({2-[((1R,2S)-1-Amino-2-vinyl-cyclopropanecarbonyl)-sulfamoyl]-phenylcarbamoyl}-methoxy)-ethoxy]-propionic acid methyl ester, 107 mg (0.20 mmol) (2S,4R)-4-[2-(2-Isopropylamino-thiazol-4-yl)-7-methoxy-quinolin-4-yloxy]-pyrrolidine-1,2-dicarboxylic acid 1-tert-butyl ester, 115 mg (0.30 mmol) HATU and 78 mg (0.61 mmol) DIPEA in 6 mL DCM.

HPLC (method A) t_{R}=5.05 min

TLC, Rf (CH₂Cl₂/MeOH 9:1)=0.35

MS (method D): 980 [M+]

### Step 5

### (2S,4R)-2-[(1R,2S)-1-(2-{2-[2-(2-Carboxy-ethoxy)-ethoxy]-acetylamino}-benzenesulfonylamino-carbonyl)-2-vinyl-cyclopropylcarbamoyl]-4-[2-(2-isopropylamino-thiazol-4-yl)-7-methoxy-quinolin-4-yloxy]-pyrrolidine-1-carboxylic acid tert-butyl ester

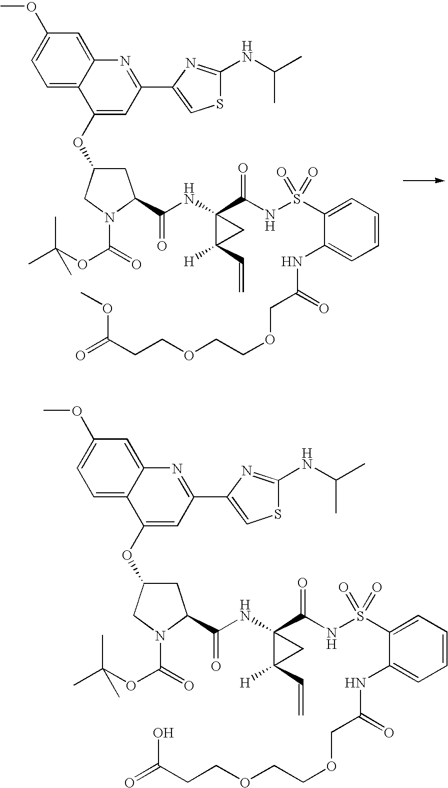

The title compound is prepared analogously as described for the title compound in Example 2 (step 2) using 110 mg (0.10 mmol) (2S,4R)-4-[2-(2-Isopropylamino-thiazol-4-yl)-7-methoxy-quinolin-4-yloxy]-2-[(1R,2S)-1-(2-{2-[2-(2-methoxycarbonyl-ethoxy)-ethoxy]-acetylamino}-benzenesulfonylaminocarbonyl)-2-vinyl-cyclopropylcarbamoyl]-pyrrolidine-1-carboxylic acid tert-butyl ester (TFA-salt) and 43 mg (1.0 mmol) LiOH in 16 mL THF/MeOH/H₂O (2:1:1).

HPLC (method A) t_{R}=4.73 min

TLC, Rf (CH₂Cl₂/MeOH/H₂O/AcOH 90:10:1:0.5)=0.40

MS (method D): 966 [M+]

### Step 6

### 3-{2-[(2-{[(1R,2S)-1-({(2S,4R)-4-[2-(2-Isopropylamino-thiazol-4-yl)-7-methoxy-quinolin-4-yloxy]-pyrrolidine-2-carbonyl}-amino)-2-vinyl-cyclopropanecarbonyl]-sulfamoyl}-phenylcarbomoyl)-methoxy]-ethoxy}-propionic acid

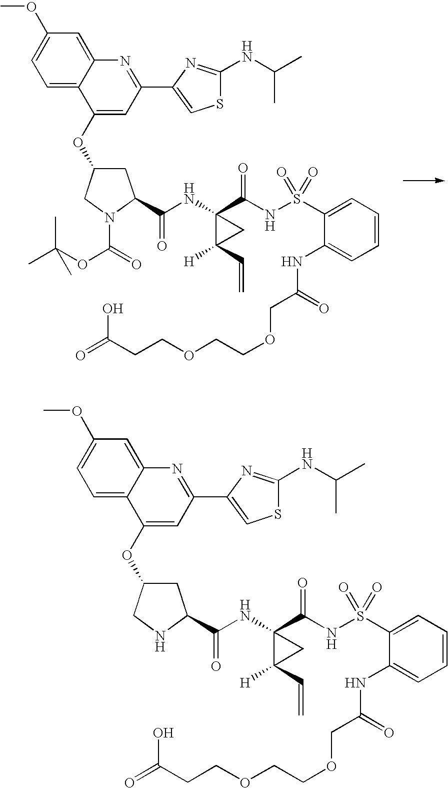

The title compound is prepared analogously as described for the title compound in Example 2 (step 3) using 96 mg (0.10 mmol) ((2S,4R)-2-[(1R,2S)-1-(2-{2-[2-(2-Carboxy-ethoxy)-ethoxy]-acetylamino}-benzenesulfonylamino-carbonyl)-2-vinyl-cyclopropylcarbamoyl]-4-[2-(2-isopropylamino-thiazol-4-yl)-7-methoxy-quinolin-4-yloxy]-pyrrolidine-1-carboxylic acid tert-butyl ester (TFA-salt) and 0.5 mL TFA in 6 mL DCM.

HPLC (method A) t_{R}=3.92 min

TLC, Rf (CH₂Cl₂/MeOH 9:1)=0.42

MS (method D): 866 [M+]

### Example 17

### 4-Fluoro-1,3-dihydro-isoindole-2-carboxylic acid (8S,10R)-5-[(1R,2S)-1-carbonylamino-2-vinyl-cyclopropyl]-2,2,4,7,13-pentaoxo-16,19-dioxa-2λ*6*-thia-3,6,12,22-tetraaza-tricyclo[21.4.0.0*8,12*]heptacosa-1(27),23,25-trien-10-yl ester

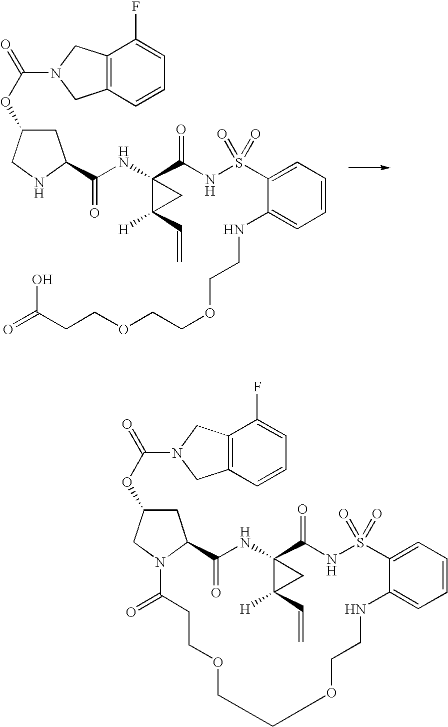

The title compound is prepared analogously as described for the title compound in Example 2 using 56 mg (0.05 mmol) 4-Fluoro-1,3-dihydro-isoindole-2-carboxylic acid (3R,5S)-5-[(1R,2S)-1-(2-{2-[2-(2-carboxy-ethoxy)-ethoxy}-ethylamino}-benzenesulfonylaminocarbonyl)-2-vinyl-cyclopropylcarbamoyl]-pyrrolidin-3-yl ester (TFA-salt), 70 mg (0.54 mmol) DIPEA and 103 mg (0.27 mmol) HATU in 50 mL DCM and 1 mL DMF.

HPLC (method A) t_{R}=4.52 min

TLC, Rf (CH₂Cl₂/MeOH 9:1)=0.45

MS (method D): 700 [M+]

### Preparation of 4-Fluoro-1,3-dihydro-isoindole-2-carboxylic acid (3R,5S)-5-[(1R,2S)-1-(2-{2-[2-(2-carboxy-ethoxy)-ethoxy]-ethylamino}-benzenesulfonylaminocarbonyl)-2-vinyl-cyclopropyl-carbamoyl]-pyrrolidin-3-yl ester

### Step 1

### 4-Fluoro-1,3-dihydro-isoindole-2-carboxylic acid (3R,5S)-1-tert-butoxycarbonyl-5-[(1R,2S)-1-(2-{2-[2-(2-methoxycarbonyl-ethoxy)-ethoxyl-ethylamino}-benzenesulfonylaminocarbonyl)-2-vinyl-cyclopropylcarbamoyl]-pyrrolidin-3-yl ester

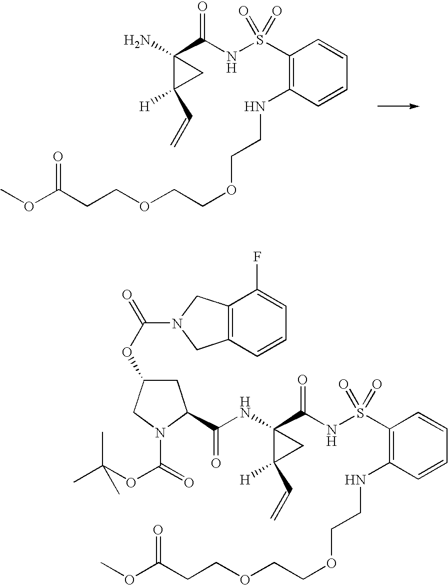

The title compound is prepared analogously as described for the title compound in Example 2 (step 1) using 235 mg (0.34 mmol) 3-[2-(2-{2-[((1R,2S)-1-Amino-2-vinyl-cyclopropanecarbonyl)-sulfamoyl]-phenylamino}-ethoxy)-ethoxy]-propionic acid methyl ester, 136 mg (0.34 mmol) ((2S,4R)-4-(4-Fluoro-1,3-dihydro-isoindole-2-carbonyloxy)-pyrrolidine-1,2-dicarboxylic acid 1-tert-butyl ester, 196 mg (0.52 mmol) HATU and 134 mg (1.0 mmol) DIPEA in 20 mL DCM.

HPLC (method A) t_{R}=5.08 min

TLC, Rf (CH₂Cl₂/MeOH 19:1)=0.31

MS (method D): 832 [M+]

### Step 2

### 4-Fluoro-1,3-dihydro-isoindole-2-carboxylic acid (3R,5S)-1-tert-butoxycarbonyl-5-[(1R,2S)-1-(2-{2-[2-(2-carboxy-ethoxy)-ethoxy]-ethylamino}-benzenesulfonylaminocarbonyl)-2-vinyl-cyclopropylcarbamoyl]-pyrrolidin-3-yl ester

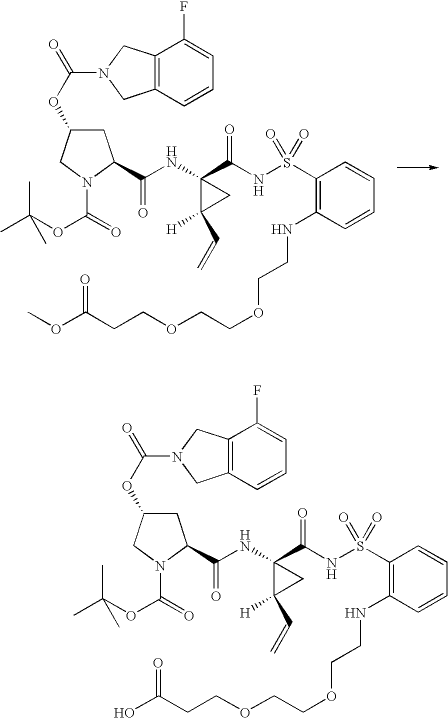

The title compound is prepared analogously as described for the title compound in Example 2 (step 2) using 170 mg (0.18 mmol) 4-Fluoro-1,3-dihydro-isoindole-2-carboxylic acid (3R,5S)-1-tert-butoxycarbonyl-5-[(1R,2S)-1-(2-{2-[2-(2-methoxycarbonyl-ethoxy-ethoxy]-ethylamino}-benzene-sulfonylaminocarbonyl)-2-vinyl-cyclopropylcarbamoyl]-pyrrolidin-3-yl ester (TFA-salt) and 76 mg (1.8 mmol) LiOH in 20 mL THF/MeOH/H₂O (2:1:1).

HPLC (method A) t_{R}=4.78 min

TLC, Rf (CH₂Cl₂/MeOH 9:1)=0.33

MS (method D): 818 [M+]

### Step 3

### 4-Fluoro-1,3-dihydro-isoindole-2-carboxylic acid (3R,5S)-5-[(1R,2S)-1-(2-{2-[2-(2-carboxy-ethoxy)-ethoxy]-ethylamino}-benzenesulfonylaminocarbonyl)-2-vinyl-cyclopropylcarbamoyl]-pyrrolidin-3-yl ester

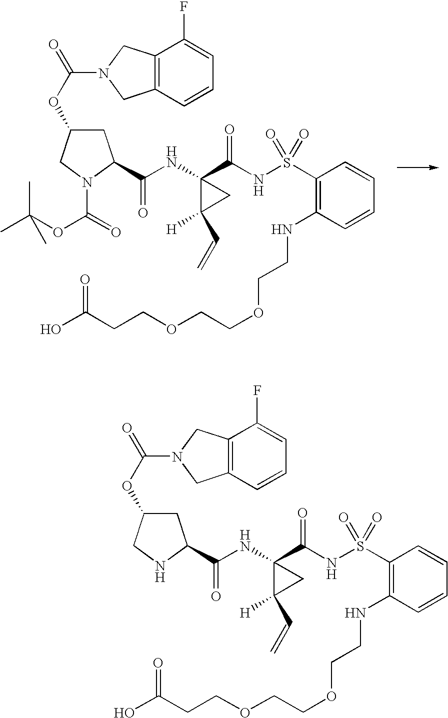

The title compound is prepared analogously as described for the title compound in Example 2 (step 3) using 52 mg (0.06 mmol) 4-Fluoro-1,3-dihydro-isoindole-2-carboxylic acid (3R,5S)-1-tert-butoxycarbonyl-5-[(1R,2S)-1-(2-{2-[2-(2-carboxy-ethoxy)-ethoxy]-ethylamino}-benzenesulfonylamino-carbonyl)-2-vinyl-cyclopropylcarbamoyl]-pyrrolidin-3-yl-ester (TFA-salt) and 0.2 mL TFA in 3 mL DCM.

HPLC (method A) t_{R}=3.85 min

TLC, Rf (CH₂Cl₂/MeOH 9:1)=0.25

MS (method D): 718 [M+]

### Example 18

### 4-Fluoro-1,3-dihydro-isoindole-2-carboxylic acid (8S,10R)-5-1(1R,2S)-1-carbonylamino-2-vinyl-cyclopropyl]-2,2,4,7,13-pentaoxo-15,19-dioxa-2λ*6*-thia-3,6,12,22-tetraaza-tricyclo[21.4.0.0*8,12*]heptacosa-1(27),23,25-trien-10-yl ester

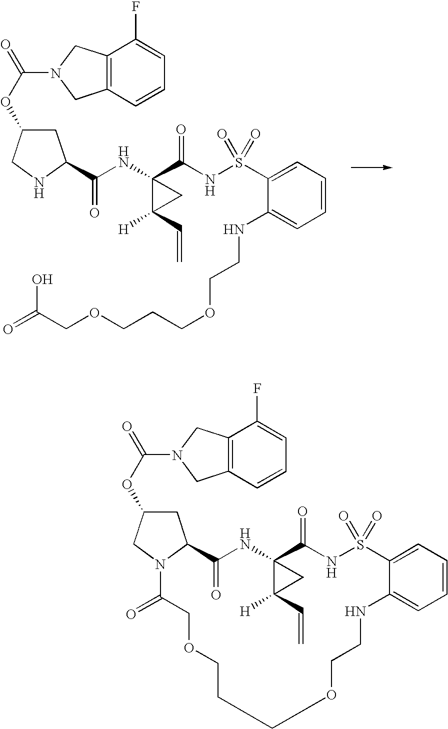

The title compound is prepared analogously as described for the title compound in Example 2 using 8 mg (0.008 mmol) 4-Fluoro-1,3-dihydro-isoindole-2-carboxylic acid (3R,5S)-5-((1R,2S)-1-{2-[2-(3-carboxymethoxy-propoxy)-ethylamino]-benzenesulfonylaminocarbonyl}-2-vinyl-cyclopropylcarbamoyl)-pyrrolidin-3-yl ester (TFA-salt), 10 mg (0.08 mmol) DIPEA and 15 mg (0.04 mmol) HATU in 25 mL DCM and 0.5 mL DMF.

HPLC (method A) t_{R}=4.63 min

TLC, Rf (CH₂Cl₂/MeOH 9:1)=0.54

MS (method D): 700 [M+]

### Preparation of 4-Fluoro-1,3-dihydro-isoindole-2-carboxylic acid (3R,5S)-5-((1R,2S)-1-{2-[2-(3-carboxymethoxy-propoxy)-ethylamino]-benzenesulfonylaminocarbonyl}-2-vinyl-cyclopropyl-carbamoyl)-pyrrolidin-3-yl ester

### Step 1

### (3-Allyloxy-propoxy)-acetic acid

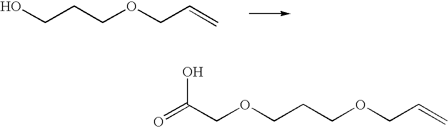

To an ice-cold solution of 7.8 g (67 mmol) 3-Allyloxy-propan-1-ol (prepared according to Synth. Comm. 1992, 22, 189-200) in 250 mL abs. THF is added 12.7 g (61 mmol) Sodium iodocaetate followed by 5.4 g (134 mmol) NaH (60% suspension in mineral oil). The ice-bath is removed and the reaction is refluxed for 5 h. After cooling to RT the reaction is quenched by addition of water and THF is removed in vacuo. The aq. phase is adjusted to pH 1 with 4 N HCl and extracted with DCM. The organic phase is washed with brine, dried with Na₂SO₄, filtered, the solvent is removed in vacuo and the residue is purified by FC on silica (eluent: CH₂Cl₂/MeOH 85:15) to give the title compound as a yellow oil.

TLC, Rf (CH₂Cl₂/MeOH 85:15)=0.62

MS (method D): 175 [M+H]

### Step 2

### (3-Allyloxy-propoxy)-acetic acid methyl ester

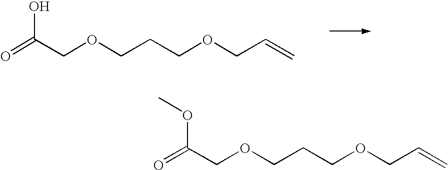

To a solution of 7.5 g (43 mmol) (3-Allyloxy-propoxy)-acetic acid in 300 mL acetone is added 6.9 g (68 mmol) KHCO₃ followed by 6.7 mL (108 mmol) Iodomethane and the reaction is refluxed for 3 h. Additional 6.7 mL (108 mmol) Iodomethane is added and reflux is continued for 3 h. A third portion of 6.7 mL (108 mmol) Iodomethane is added and the mixture is refluxed overnight. After cooling to RT the reaction mixture is filtered and the solvent is removed in vacuo. The residue is taken up in EtOAc, washed with sat. NaHCO₃-solution and brine, dried with Na₂SO₄, filtered and the solvent is removed in vacuo. The residue is used without further purification.

TLC, Rf (CH₂Cl₂/MeOH 19:1)=0.78

MS (method D): 206 [M+18]

### Step 3

### [3-(2-Oxo-ethoxy)-propoxy]-acetic acid methyl ester

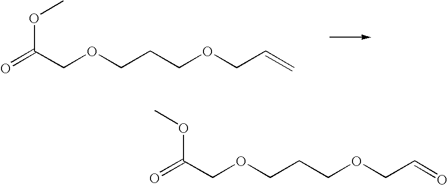

A solution of 2.0 g (11.0 mmol) (3-Allyloxy-propoxy)-acetic acid methyl ester in 200 mL

DCM is cooled to −78° C. Ozone is bubbled through until a blue color appears (˜30 min). Oxygen is bubbled through the mixture for 2 min to remove excess of ozone, 1.0 mL (14 mmol) Dimethylsulfide is added and stirring is continued for 1 h at −78° C. After warming to RT, the solvent is removed in vacuo and the residue is used without further purification.

### Step 4

### [3-(2-{2-[((1R,2S)-1-tert-Butoxycarbonylamino-2-vinyl-cyclopropanecarbonyl)-sulfamoyl]-phenylamino}-ethoxy)-propoxy]-acetic acid methyl ester

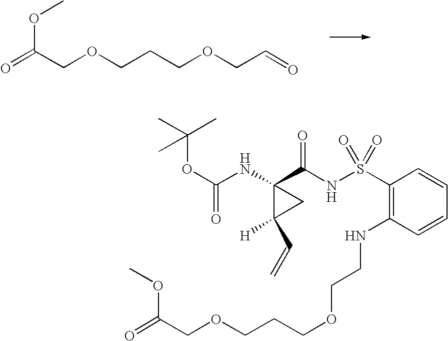

The title compound is prepared analogously as described for the title compound in Example 11 (step 3) using 1.9 g (5 mmol) [(R1R,2S)-1-(2-Amino-benzenesulfonylaminocarbonyl)-2-vinyl-cyclopropyl]carbamic acid tert-butyl ester, 2.56 g crude [3-(2-Oxo-ethoxy)-propoxy]-acetic acid methyl ester (from the previous step), 3.3 g (15 mmol) NaBH(OAc)₃ and 0.90 g (15 mmol) AcOH in 150 mL 1.2 DCE.

MS (method D): 556 [M+]

### Step 5

### [3-(2-{2-[((1R,2S)-1-Amino-2-vinyl-cyclopropanecarbonyl)-sulfamoyl]-phenylamino}-ethoxy)-propoxy]-acetic acid methyl ester

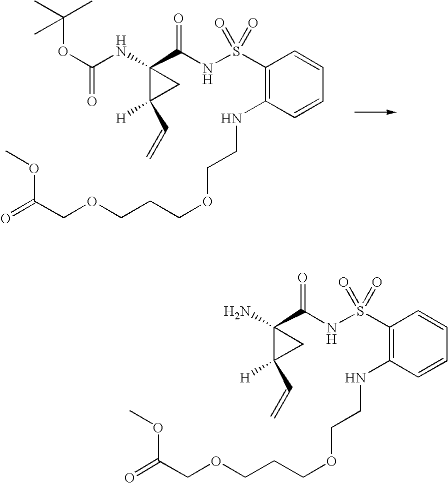

The title compound is prepared analogously as described for the title compound in Example 11 (step 4) using 1.78 g (3.2 mmol) [3-(2-{2-[((1R,2S)-1-tert-Butoxycarbonylamino-2-vinyl-cyclopropane-carbonyl)-sulfamoyl]-phenylamino}-ethoxy)-propoxy]-acetic acid methyl ester and 5 mL TFA in 25 mL DCM.

MS (method D): 456 [M+]

### Step 6

### 4-Fluoro-1,3-dihydro-isoindole-2-carboxylic acid (3R,5S)-1-tert-butoxycarbonyl-5-((1R,2S)-1-{2-[2-(3-methoxycarbonylmethoxy-propoxy)-ethylamino]-benzenesulfonylaminocarbonyl }-2-vinyl-cyclopropylcarbamoyl)-pyrrolidin-3-yl ester

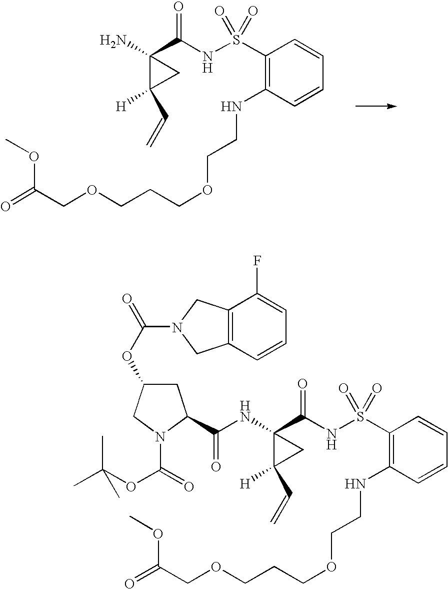

The title compound is prepared analogously as described for the title compound in Example 2 (step 1) using 520 mg (0.38 mmol) [3-(2-{2-[((1R,2S)-1-Amino-2-vinyl-cyclopropanecarbonyl)-sulfamoyl]-phenylamino}-ethoxy)-propoxy]-acetic acid methyl ester (TFA-alt), 150 mg (0.38 mmol) (((2S,4R)-4-(4-Fluoro-1,3-dihydro-isoindole-2-carbonyloxy)-pyrrolidine-1,2-dicarboxylic acid 1-tert-butyl ester, 217 mg (0.57 mmol) HATU and 295 mg (2.3 mmol) DIPEA in 10 mL DCM.

HPLC (method A) t_{R}=5.23 min

TLC, Rf (CH₂Cl₂/MeOH 9:1)=0.63

MS (method D): 832 [M+]

### Step 7

### 4-Fluoro-1,3-dihydro-isoindole-2-carboxylic acid (3R,5S)-1-tert-butoxycarbonyl-5-((1R,2S)-1-{2-[2-(3-carboxymethoxy-propoxy)-ethylamino]-benzenesulfonylaminocarbonyl}-2-vinyl-cyclopropylcarbamoyl)-pyrrolidin-3-yl ester

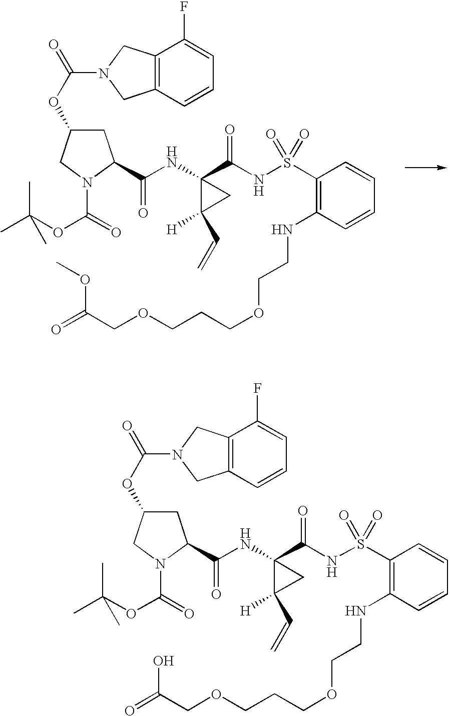

The title compound is prepared analogously as described for the title compound in Example 2 (step 2) using 28 mg (0.015 mmol) 4-Fluoro-1,3-dihydro-isoindole-2-carboxylic acid (3R,5S)-1-tert-butoxycarbonyl-5-((1R,2S)-1-{2-[2-(3-methoxycarbonylmethoxy-propoxy)-ethylamino]-benzene-sulfonylaminocarbonyl}-2-vinyl-cyclopropylcarbamoyl)-pyrrolidin-3-yl ester (TFA-salt) and 7 mg (0.3 mmol) LiOH in 20 mL THF/MeOH/H₂O (2:1:1).

HPLC (method A) t_{R}=4.82 min

TLC, Rf (CH₂Cl₂/MeOH 9:1)=0.29

MS (method D): 818 [M+]

Step 8

### 4-Fluoro-1,3-dihydro-isoindole-2-carboxylic acid (3R,5S)-5-((1R,2S)-1-{2-[2-(3-carboxymethoxy-propoxy)-ethylamino]-benzenesulfonylaminocarbonyl}-2-vinyl-cyclopropylcarbamoyl)-pyrrolidin-3-yl ester

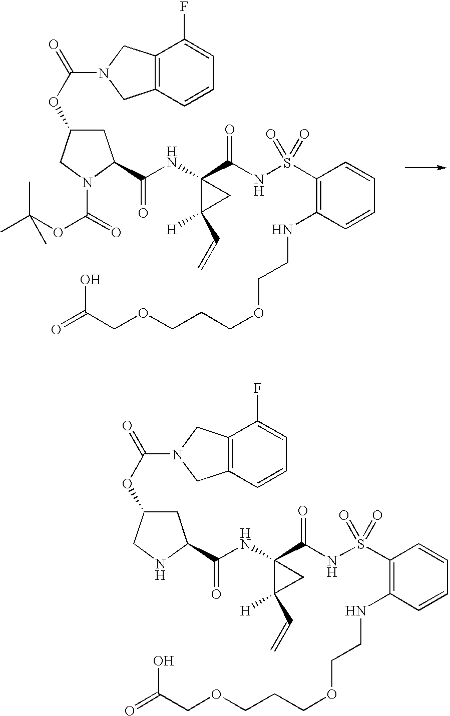

The title compound is prepared analogously as described for the title compound in Example 2 (step 3) using 8 mg (0.01 mmol) (4-Fluoro-1,3-dihydro-isoindole-2-carboxylic acid (3R,5S)-1-tert-butoxycarbonyl-5-((1R,2S)-1-{2-[2-(3-carboxymethoxy-propoxy)-ethylamino]-benzenesulfonyl-aminocarbonyl}-2-vinyl-cyclopropylcarbamoyl)-pyrrolidin-3-yl ester (TFA-salt) and 0.2 mL TFA in 1 mL DCM.

HPLC (method A) t_{R}=3.88 min

TLC, Rf (CH₂Cl₂/MeOH 9:1)=0.44

MS (method D): 718 [M+]

### Example 19

### (8S,10R)-10-[2-(2-Isopropylamino-thiazol-4-yl)-7-methoxy-quinolin-4-yloxy]-5-[(1R,2S)-1-carbonylamino-2-vinyl-cyclopropyl]-17-methyl-2,2-dioxo-2λ*6*-thia-3,6,12,17,22-pentaaza-tricyclo[21.4.0.0*8,12*]heptacosa-1(27),23,25-triene-4,7,13-trione

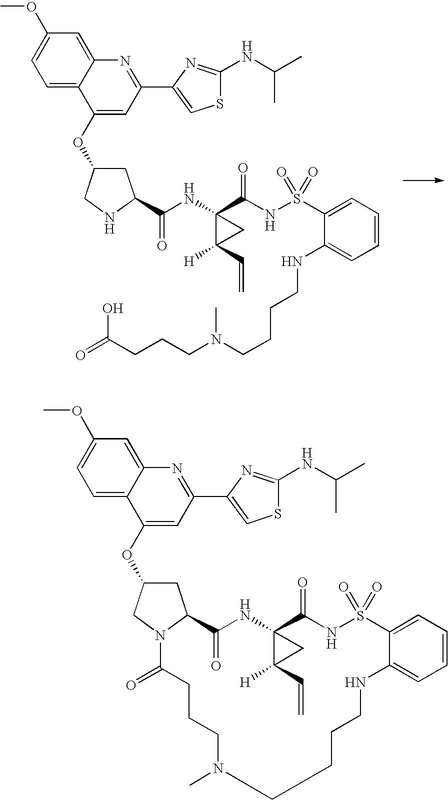

The title compound is prepared analogously as described for the title compound in Example 2 using 330 mg (0.16 mmol) 4-{[4-(2-{[(1R,2S)-1-({(2S,4R)-4-[2-(2-Isopropylamino-thiazol-4-yl)-7-methoxy-quinolin-4-yloxy]-pyrrolidine-2-carbonyl}-amino)-2-vinyl-cyclopropanecarbonyl]-sulfamoyl}-phenylamino)-butyl]methyl-amino -butyric acid (TFA-salt), 0.29 mL (1.64 mmol) DIPEA and 312 mg (0.82 mmol) HATU in 40 mL DCM and 1 mL DMF.

HPLC (method A) t_{R}=4.24 min

MS (method D): 845 [M+]

### Preparation of 4-{[4-(2-{[(1R,2S)-1-({(2S,4R)-4-[2-(2-Isopropylamino-thiazol-4-yl)-7-methoxy-4-yloxy]-pyrrolidine-2-carbonyl)-amino)-2-vinyl-cyclopropanecarbonyl]-sulfamoyl}-phenylamino)-butyl]-methyl-amino}-butyric acid

Step 1

### 4-Methylamino-butyric acid methyl ester

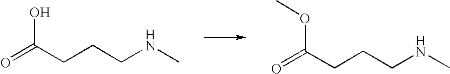

A solution of 2.3 g (15 mmol) 4-Methylamino-butyric acid hydrochloride and 25 mL (31 mmol) HCl (1.25 M in MeOH) in 150 mL MeOH is stirred overnight at RT. The solvent is removed in vacuo and the residue is used without further purification.

MS (method D): 132 [M+H]

Step 2

### 4-{[4-(tert-Butyl-dimethyl-silanyloxy)-butyl]-methyl-amino}-butyric acid methyl ester

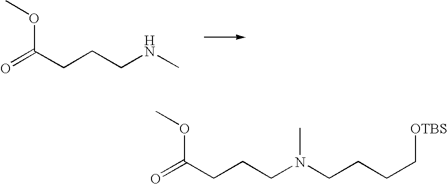

The title compound is prepared analogously as described for the title compound in Example 11 (step 3) using 1.6 g (9.5 mmol) 4-Methylamino-butyric acid methyl ester hydrochloride, 1.93 g (9.5 mmol) 4-(tert-Butyl-dimethyl-silanyloxy)-butyraldehyde (prepared according to J. Org. Chem. 2005, 70(6), 2097), 4.50 g (19 mmol) NaBH(OAc)₃ and 1.1 mL (19 mmol) AcOH in 100 mL 1.2 DCE.

MS (method D): 318 [M+]

Step 3

### 4-[(4-Hydroxy-butyl)-methyl-amino]-butyric acid methyl ester

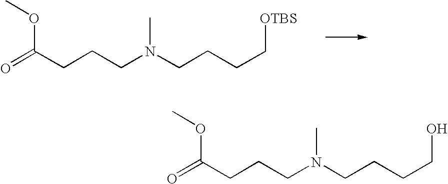

To an ice-cold solution of 2.1 g (6.6 mmol) 4-{[4-(tert-Butyl-dimethyl-silanyloxy)-butyl]-methyl-amino}-butyric acid methyl ester in 10 mL abs. THF is slowly added 7.9 mL (7.9 mmol) TBAF (1M in THF). After 2 h at RT additional 2 mL TBAF is added stirring is continued for 2 h, the solvent is removed in vacuo and the residue is purified by FC on silica (eluent: TBME/MeOH/NH₄OH 90:10:1) to give the title compound as a brown oil.

MS (method D): 204 [M+H]

Step 4

### 4-[Methyl-(4-oxo-butyl)-amino]-butyric acid methyl ester

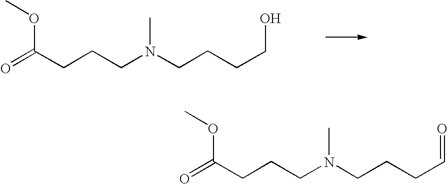

To a solution of 100 mg (0.47 mmol) 4-[(4-Hydroxy-butyl)-methyl-amino]-butyric acid methyl ester in 2 mL DCM is added 220 mg (0.98 mmol) PCC. After stirring overnight at RT, the solvent is removed in vacuo and the residue is purified by FC on silica (eluent: TBME/MeOH/NH₄OH 85:15:1) to give the title compound as a brown oil.

TLC, Rf (TBME/MeOH/NR₄OH 90:10:1)=0.30

Step 5

### 4-[(4-{2-[((1R,2S)-1-tert-Butoxycarbonylamino-2-vinyl-cyclopropanecarbonyl)-sulfamoyl]-phenylamino}-butyl)-methyl-amino}-butyric acid methylester

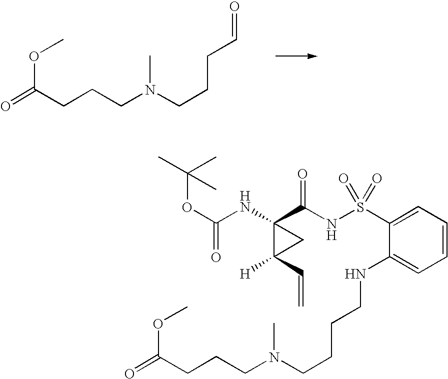

The title compound is prepared analogously as described for the title compound in Example 11 (step 3) using 2.0 g (5.2 mmol) (1R,2S)-1-(2-Amino-benzenesulfonylaminocarbonyl)-2-vinyl-cyclopropyl]-carbamic acid tert-butyl ester, 1.27 g (6.3 mmol) 4-[Methyl-(4-oxo-butyl)-amino]-butyric acid methyl ester, 3.1 g (13 mmol) NaBH(OAc)₃ and 0.90 mL (16 mmol) AcOH in 80 mL 1,2-Dichloroethane.

HPLC (method B) t_{R}=5.67 min

MS (method D): 567 [M+]

Step 6

4-[(4-{2-[((1R,2S)-1-Amino-2-vinyl-cyclopropanecarbonyl)-sulfamoyl]-phenylamino}-butyl)-methyl-amino]-butyric acid methyl ester

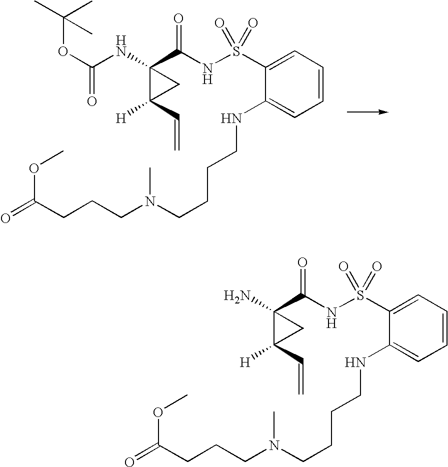

The title compound is prepared analogously as described for the title compound in Example 11 (step 4) using 210 mg (0.37 mmol) (4-[(4-{2-[((1R,2S)-1-tert-Butoxycarbonylamino-2-vinyl-cyclopropanecarbonyl)-sulfamoyl]-phenylamino}-butyl)-methyl-amino]-butyric acid methylester and 1.4 mL TFA in 15 mL DCM.

MS (method D): 467 [M+]Step 7

### (2S,4R)-2-[(1R,2S)-1-(2-{4-[(3-Methoxycarbonyl-propyl)-methyl-amino]--butylamino}-benzenesulfonylaminocarbonyl)-2-vinyl-cyclopropylcarbamoyl]-4-[7-methoxy-2-(2-isopropylamino-thiazol-4-yl)-quinolin-4-yloxy]-pyrrolidine-1-carboxylic acid tert-butyl ester

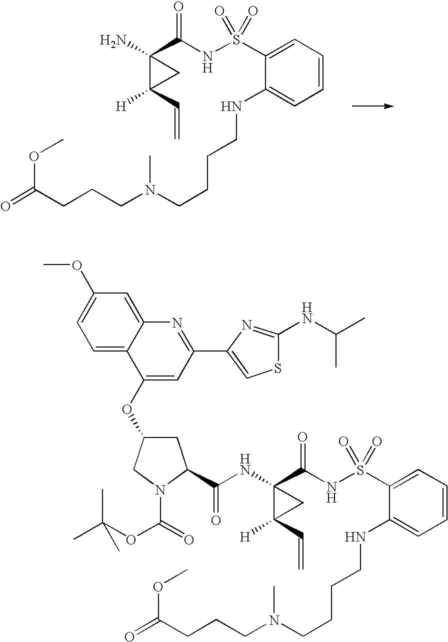

The title compound is prepared analogously as described for the title compound in Example 2 (step 1) using 244 mg (0.37 mmol) 4-[(4-{2-[((1R,2S)-1-Amino-2-vinyl-cyclopropanecarbonyl)-sulfamoyl]-phenylamino}-butyl)-methyl-amino}-butyric acid methyl ester, 210 mg (0.37 mmol) (2S,4R)-4-[2-(2-Isopropylamino-thiazol-4-yl)-7-methoxy-quinolin-4-yloxy]-pyrrolidine-1,2-dicarboxylic acid 1-tert-butyl ester, 212 mg (0.56 mmol) HATU and 0.39 mL (2.23 mmol) DIPEA in 5 mL DCM.

HPLC (method B) t_{R}=5.95 min

MS (method D): 977.5 [M+]

Step 8

### (2S,4R)-2-[(1R,2S)-1-(2-{4-[(3-Carboxy-propyl)-methyl-amino]-butylamino}-benzenesulfonylaminocarbonyl)-2-vinyl-cyclopropylcarbamoyl-4-[2-(2-isopropylamino-thiazol-4-yl)-7-methoxy-quinolin-4-yloxy]-pyrrolidine-1-carboxylic acid tert-butyl ester

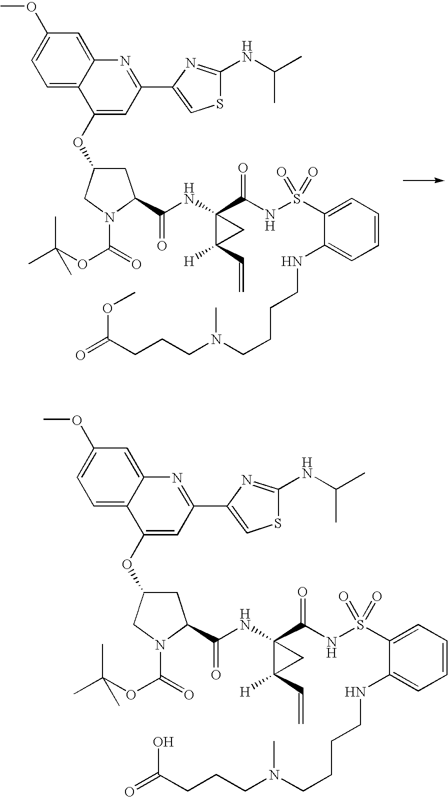

The title compound is prepared analogously as described for the title compound in Example 2 (step 2) using 160 mg (0.16 mmol) (2S,4R)-2-[(1R,2S)-1-(2-{4-[(3-Methoxycarbonyl-propyl)-methyl-amino]-butylamino}-benzenesulfonylaminocarbonyl)-2-vinyl-cyclopropylcarbamoyl]-4-[7-methoxy-2-(2-isopropylamino-thiazol-4-yl)-quinolin-4-yloxy]-pyrrolidine-1-carboxylic acid tert-butyl ester (TFA-salt) and 35 mg (0.82 mmol) LiOH in 10 mL THF/MeOH/H₂O (2:1:1).

HPLC (method B) t_{R}=6.06 min

MS (method D):963 [M+]

Step 9

### 4-{[4-(2-{[(1R,2S)-1-({(2S,4R)-4-[2-(2-Isopropylamino-thiazol-4-yl)-7-methoxy-quinolin-4-yloxy]-pyrrolidine-2-carbonyl}-amino)-2-vinyl-cyclopropanecarbonyl]-sulfamoyl}-phenylamino)-butyl]-methyl-amino}-butyric acid

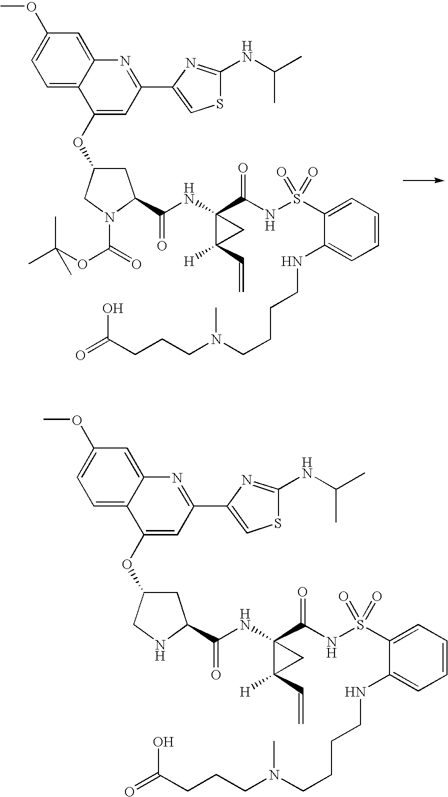

The title compound is prepared analogously as described for the title compound in Example 2 (step 3) using 176 mg (0.16 mmol) (2S,4R)-2-[(1R,2S)-1-(2-{4-[(3-Carboxy-propyl)-methyl-amino}-butylamino}-benzenesulfonylaminocarbonyl)-2-vinyl-cyclopropylcarbamoyl]-4-[2-(2-isopropylamino-thiazol-4-yl)-7-methoxy-quinolin-4-yloxy]-pyrrolidine-1-carboxylic acid tert-butyl ester (TFA-salt) and 0.8 mL TFA in 10 mL DCM.

HPLC (method B) t_{R}=5.66 min

MS (method D): 863 [M+]

### Example 20

### 4-Fluoro-1,3-dihydro-isoindole-2-carboxylic acid (8S,10R)-5-[(1R,2S)-1-carbonylamino-2-vinyl-cyclopropyl]-17-methyl-2,2,4,7,13-pentaoxo-2λ*6*-thia-3,6,12,17,22-pentaaza-tricyclo[21.4.0.0*8,12*]heptacosa-1(27),23,25-trien-10-yl ester

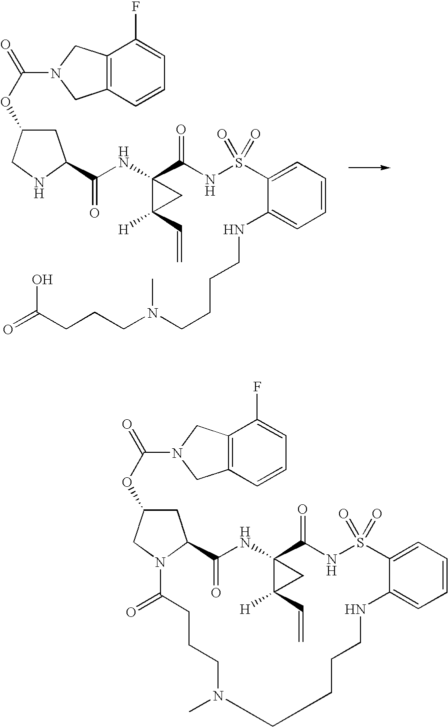

The title compound is prepared analogously as described for the title compound in Example 2 using 300 mg (0.19 mmol) 4-Fluoro-1,3-dihydro-isoindole-2-carboxylic acid (3R,5S)-5-[(1R,2S)-1-(2-{4-[(3-carboxy-propyl)-methyl-amino]-butylamino}-benzenesulfonylaminocarbonyl)-2-vinyl-cyclopropylcarbamoyl]-pyrrolidin-3-yl ester (TFA-salt), 0.32 mL (1.9 mmol) DIPEA and 361 mg (0.95 mmol) HATU in 50 mL DCM and 1 mL DMF.

HPLC (method A) t_{R}=4.10 min

MS (method D): 711 [M+]

### Preparation of 4-Fluoro-1,3-dihydro-isoindole-2-carboxylic acid (3R,5S)-5-[(1R,2S)-1-(2-{4-[(3-carboxy-propyl)-methyl-amino]-butylamino}-benzenesulfonylaminocarbonyl)-2-vinyl-cyclopropylcarbamoyl]-pyrrolidin-3-yl ester

Step 1

### 4-Fluoro-1,3-dihydro-isoindole-2-carboxylic acid (3R,5S)-1-tert-butoxycarbonyl-5-[(1R,2S)-1-(2-{4-[(3-methoxycarbonyl-propyl)-methyl-amino]-butylamino}-benzenesulfonylaminocarbonyl)-2-vinyl-cyclopropylcarbamoyl]-pyrrolidin-3-yl ester

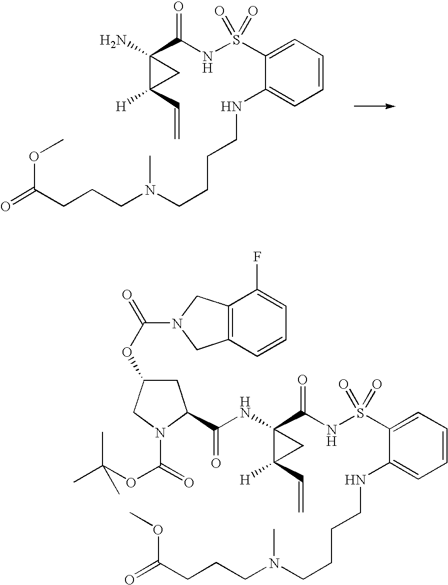

The title compound is prepared analogously as described for the title compound in Example 2 (step 1) using 250 mg (0.29 mmol) 4-[(4-{2-[((1R,2S)-1-Amino-2-vinyl-cyclopropanecarbonyl)-sulfamoyl]-phenylamino }-buty)-methyl-amino]-butyric acid methyl ester, 110 mg (0.28 mmol) (2S,4R)-4-(4-Fluoro-1,3-dihydro-isoindole-2-carbonyloxy)-pyrrolidine-1,2-dicarboxylic acid 1-tert-butyl ester, 159 mg (0.42 mmol) HATU and 0.29 mL (1.7 mmol) DIPEA in 5 mL DCM.

HPLC (method B) t_{R}=6.52 min

MS (method D): 843 [M+]

Step 2

### 4-Fluoro-1,3-dihydro-isoindole-2-carboxylic acid (3R,5S)-1-tert-butoxycarbonyl-5-[(1R,2S)-1-(2-{4-[(3-methoxycarbonyl-propyl)-methyl-amino]-butylamino}-benzenesulfonylaminocarbonyl)-2-vinyl-cyclopropylcarbamoyl]-pyrrolidin-3-yl ester

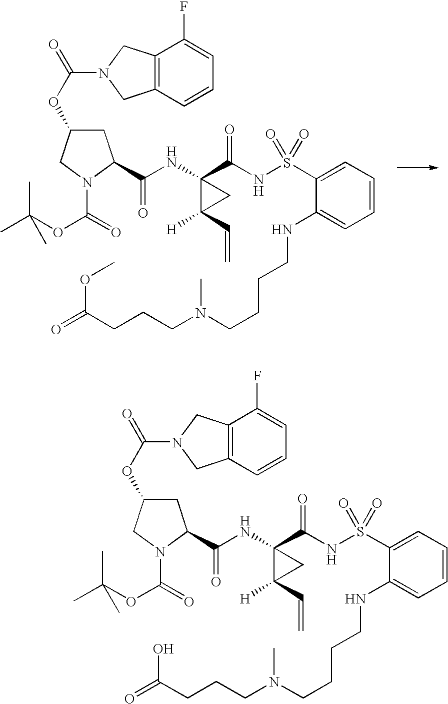

The title compound is prepared analogously as described for the title compound in Example 2 (step 2) using 160 mg (0.19 mmol) 4-Fluoro-1,3-dihydro-isoindole-2-carboxylic acid (3R,5S)-1-tert-butoxycarbonyl-5-[(1R,2S)-1-(2-{4-[(3-methoxycarbonyl-propyl)-methyl-amino]-butylamino}-benzene-sulfonylaminocarbonyl)-2-vinyl-cyclopropylcarbamoyl]-pyrrolidin-3-yl ester (TFA-salt) and 32 mg (0.76 mmol) LiOH in 10 mL THF/MeOH/H₂O (2:1:1).

HPLC (method B) t_{R}=6.31 min

MS (method D): 829 [M+]

Step 3

### 4-Fluoro-1,3-dihydro-isoindole-2-carboxylic acid (3R,5S)-5-[(1R,2S)-1-(2-{4-[(3-carboxy-propyl)-methyl-amino]-butylamino}-benzenesulfonylaminocarbonyl)-2-vinyl-cyclopropylcarbamoyl]-pyrrolidin-3-yl ester

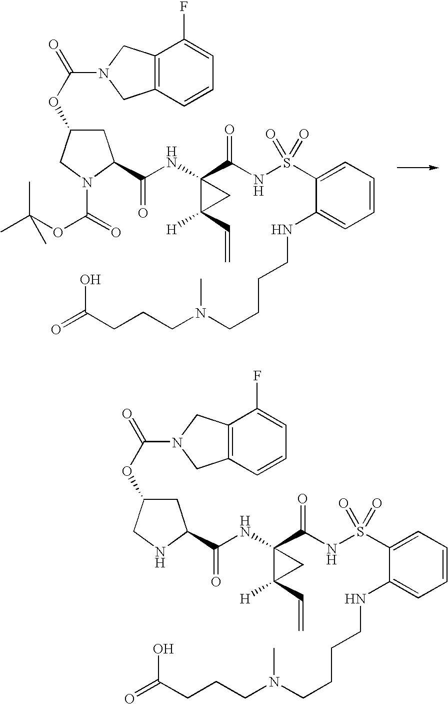

The title compound is prepared analogously as described for the title compound in Example 2 (step 3) using 190 mg (0.19 mmol) 4-Fluoro-1,3-dihydro-isoindole-2-carboxylic acid (3R,5S)-1-tert-butoxycarbonyl-5-[(1R,2S)-1-(2-{4-[(3-methoxycarbonyl-propyl)-amino]-butylamino}-benzenesulfonylaminocarbonyl)-2-vinyl-cyclopropylcarbamoyl]-pyrrolidin-3-yl ester (TFA-salt) and 0.8 mL TFA in 10 mL DCM.

HPLC (method B) t_{R}=5.94 min

MS (method D): 729 [M+]

### Example 21

### {(8S,10R,14S)-10-[2-(2-Isopropylamino-thiazol-4-yl)-7-methoxy-quinolin-4-yloxy}-5-[(1R,2S)-1-carbonylamino-2-vinyl-cyclopropyl]-2,2,4,7,13-pentaoxo-2λ*6*-thia-3,6,12,22-tetraaza-tricyclo[21.4.0.0*8,121heptacosa-1(23),24,26-trien-14-yl}-carbamic acid cyclopentyl ester

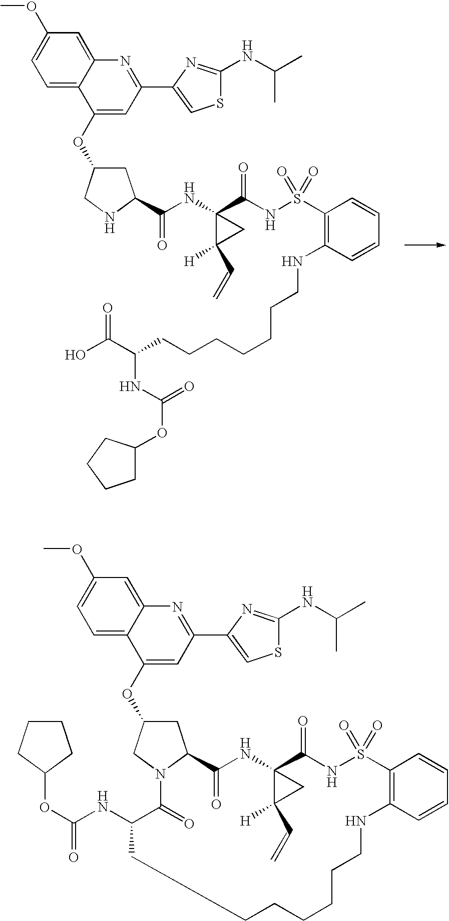

The title compound is prepared analogously as described for the title compound in Example 2 using 120 mg (0.07 mmol) (S)-2-Cyclopentyloxycarbonylamino-9-(2-{[(1R,2S)-1-({(2S,4R)-4-[2-(2-isopropyl-amino-thiazol-4-yl)-7-methoxy-quinolin-4-yloxy]-pyrrolidine-2-carbonyl}-amino)-2-vinyl-cyclopropane-carbonyl]-sulfamoyl}-phenylamino)-nonanoic acid (TFA-salt), 92 mg (0.71 mmol) DIPEA and 135 mg (0.36 mmol) HATU in 50 mL DCM and 1 mL DMF.

HPLC (method A) t_{R}=6.03 min

TLC, Rf (CH₂Cl₂/MeOH 9:1)=0.58

MS (method D): 957 [M+]

### Preparation of (S)-2-Cyclopentyloxycarbonylamino-9-(2-{[(1R,2S)-1-({(2S,4R)-4-[2-(2-isopropyl-amino-thiazol-4-yl)-7-methoxy-quinolin-4-yloxy]-pyrrolidine-2-carbonyl}-amino)-2-vinyl-cyclopropane-carbonyl]-sulfamoyl}-phenylaminoynonanoic acid

Step 1

### (2S,5R)-3,6-Diethoxy-2-hept-6-enyl-5-isopropyl-2,5-dihydro-pyrazine

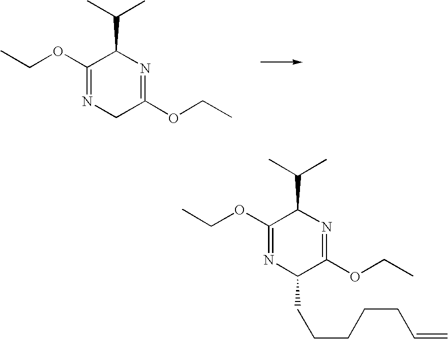

A solution of 26.2 g (123 mmol) of (R)-3,6-Diethoxy-2-isopropyl-2,5-dihydro-pyrazine in 450 ml of abs. THF under argon is cooled to −75° C. and 77 mL (123 mmol) n-BuLi (1.6 M in Toluene) is added within 45 min while the temperature is maintained below −70° C. A solution of 15 g (85 mmol) of 7-bromo-1-heptene in 80 ml of THF abs is added at −70° C. The reaction mixture is stirred for 3 h at −70° C., for 17h at −4° C. and for 3 h at RT. Ice-cold saturated NH₄Cl (70 ml) and H₂O (500 ml) are added and the resulting mixture is extracted with EtOAc (500 ml). The organic layer is washed with H₂O. The combined aqueous phases are extracted with EtOAc (500 ml). The combined organic phases are dried over Na₂SO₄, concentrated in vacuo and the residue purified by FC on silica gel. (eluent: Hexane/EtOAc 30:1) to give the title compound as a yellow oil.

TLC, Rf (Hexane/EtOAc 30:1)=0.46

MS (method D): 309 [M+H]

Step 2

### (S)-2-Amino-non-8-enoic acid ethyl ester

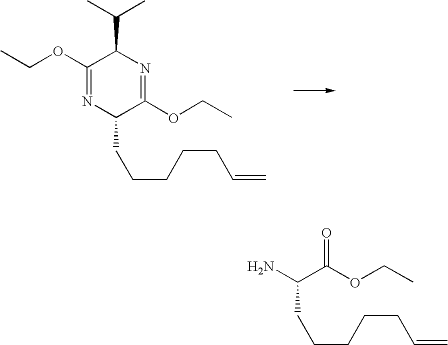

To a solution of 19 g (62 mmol) (2S,5R)-3,6-Diethoxy-2-hept-6-enyl-5-isopropyl-2,5- dihydro-pyrazine in 400 mL ACN at RT, is added 250 mL of 1N aq HCl. The reaction mixture is stirred for 2 h at RT. Saturated aq. NaHCO₃ (250 mL) is added to adjust pH 8. The reaction mixture is stirred overnight at RT and then concentrated in vacuo. The aq. phase is extracted with 500 mL of EtOAc. The organic phase is washed twice with 250 mL H₂O, dried over Na₂SO₄, concentrated in vacuo and the residue is purified on silica gel. (eluent: EtOAc). The product is distilled under high vacuum to give the title compound (S)-2-Amino-non-8-enoic acid ethyl ester as a colorless oil.

TLC, Rf (Hexane/ EtOAc 1:2)=0.21

MS (method D): 200 [M+H]

Step 3

### (S)-2-Cyclopentyloxycarboxycarbonylamino-non-8-enoic acid ethyl ester

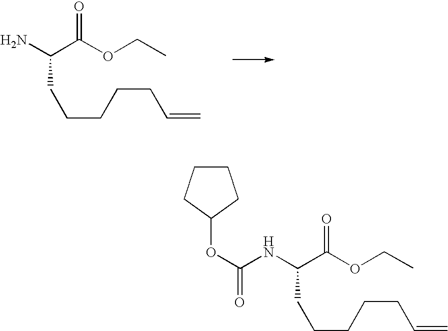

To a solution of 9.3 mL (100 mmol) of cyclopentanol in 200 mL of THF abs under nitrogen atmosphere at 10° C., is added over a 20-min period 89 mL (169 mmol)) of a phosgen solution (20% in Toluene). The reaction mixture is warmed up to RT and stirred for 2 h, while a nitrogen stream is passed through the solution, so that the reaction volume is concentrated to 150 mL. A solution of 8.0 g (41 mmol) of (S)-2-amino-non-8-enoic acid ethyl ester in 20 mL abs. THF is added at RT, followed by triethylamine added at 0° C. until pH 9.4 is adjusted. The reaction mixture is stirred for 1 h at 0° C. and concentrated in vacuo. EtOAc (500 mL) is added and the organic layer is washed 3× with H₂O (100 mL), with NaHCO₃ (100 mL) and with brine (100 mL). The organic layer is dried over Na₂SO₄, concentrated in vacuo, and the residue is purified by FC on silica gel. (Eluent: Hexane/EtOAc 7:1) to give the title compound as a yellow oil.

TLC, Rf (Hexane/ EtOAc 3:1)=0.33

MS (method D): 312 [M+H]

Step 4

### (S)-2-Cyclopentyloxycarbonylamino-non-8-enoic acid

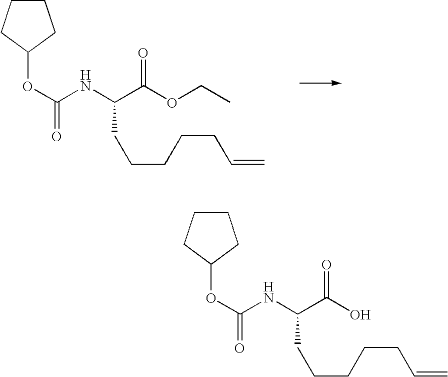

To a solution of 460 g (1.5 mol) of (S)-2-Cyclopenthyloxycarboxycarbonylamino-non-8-enoic acid ethyl ester in 4.0 L of THF 1.8 L of Methanol is added at RT. A solution of 137 g (3.25 mol) of LiOH monohydrate in 1.8 L of water is added over a 40-min period. The reaction mixture is stirred at RT for 3 h, concentrated in vacuo, taken up in H₂O (2L), washed with 10% aqueous citric acid (2.5 L) and extracted with EtOAc (2.5 L). The organic layer is washed with H₂O (2×2 L) and brine (2 L). The organic layer is dried over Na₂SO₄, concentrated in vacuo and the residue purified by FC on silica gel (eluent: Hexane/EtOAc 10:1→EtOAc) to give the title compound as a red amorphous solid.

TLC, Rf (CH2Cl2/MeOH 9:1)=0.3

MS (method D): 282 [M−H]

Step 5

### (S)-2-Cyclopentyloxycarbonylamino-non-8-enoic acid methyl ester

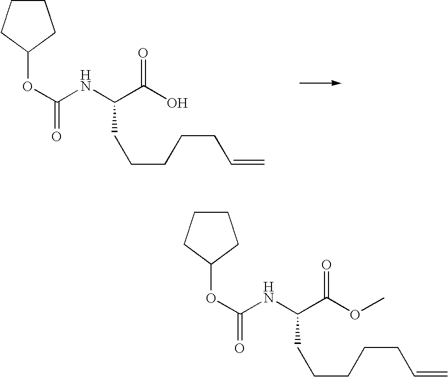

To a solution of 11.5 g (41 mmol) (S)-2-Cyclopentyloxycarbonylamino-non-8-enoic acid in 200 mL Acetone is added at rt 6.5 g (65 mmol) KHCO₃ and 14.4 g (101 mmol) Iodomethane and the reaction is refluxed for 15 h. After cooling to rt the reaction mixture is filtered, washed with Acetone and the solvent removed in vacuo. The residue is dissolved in EtOAc, washed with aq. bicarbonate and brine, dried with Na₂SO₄, filtered and the solvent is removed in vacuo. The residue is purified by FC on silica (eluent: CH₂Cl₂/MeOH 99:1→95:5) to give the title compound as a yellow oil.

HPLC (method A) t_{R}=5.29 min

TLC, Rf (CH₂Cl₂/MeOH 99:1)=0.50

MS (method D): 298 [M+H]

Step 6

### (S)-2-Cyclopentyloxycarbonylamino-9-hydroxy-nonanoic acid methyl ester

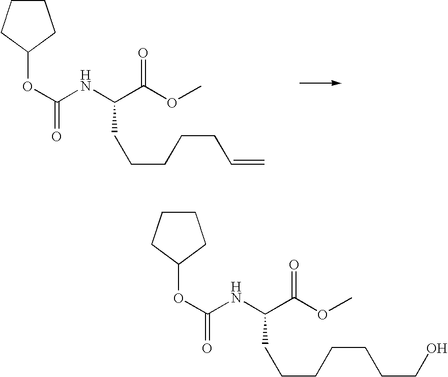

To an ice-cold solution of 8.1 g (27 mmol) (S)-2-Cyclopentyloxycarbonylamino-non-8-enoic acid methyl ester in 200 mL THF is added 82 mL (41 mmol) 9-BBN (0.5 M in THF) and the ice-bath is removed. After stirring for 2 h the reaction is cooled to 0° C. and quenched by addition of 25 mL aq. bicarbonate and 5 mL aq. 35% H₂O₂. After extraction with EtOAc, the combined organic phase is dried with Na₂SO₄, filtered and the solvent is removed in vacuo. The residue is purified by FC on silica (eluent: CH₂Cl₂/MeOH 98:2→95:5) to give the title compound as a colorless oil.

HPLC (method A) t_{R}=3.95 min

TLC, Rf (CH₂Cl₂/MeOH 19:1)=0.34

MS (method D): 316 [M+H]

Step 7

### (S)-2-Cyclopentyloxycarbonylamino-9-oxo-nonanoic acid methyl ester

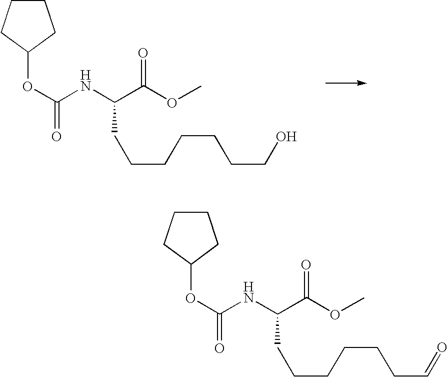

To a solution of 2.2 g (7.0 mmol) (S)-2-Cyclopentyloxycarbonylamino-9-hydroxy-nonanoic acid methyl ester in 150 mL DCM is added 2.3 g (10.5 mmol) PCC. After stirring for 4 h at rt silica is added, the reaction is filtered through a pad of Hyflo and thoroughly washed with DCM. The solvent is removed in vacuo to give the title compound as a brown oil, which is used without further purification.

TLC, Rf (CH₂Cl₂/MeOH 19:1)=0.54

MS (method D): 314 [M+H]

Step 8

### (S)-9-(2-[((1R,2S)-1-tert-Butoxycarbonylamino-2-vinyl-cyclopropanecarbonyl)-sulfamoyl]-phenylamino}-2-cyclopentyloxycarbonylamino-nonanoic acid methyl ester

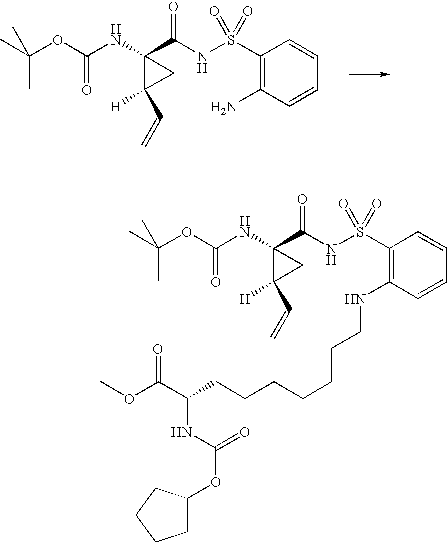

The title compound is prepared analogously as described for the title compound in Example 11 (step 3) using 0.95 g (2.5 mmol) 1R,2S)-1-(2-Amino-benzenesulfonylaminocarbonyl)-2-vinyl-cyclopropyl]-carbamic acid tert-butyl ester, 1.95 g (4.98 mmol) (S)-2-Cyclopentyloxycarbonylamino-9-oxo-nonanoic acid methyl ester, 1.58 g (7.5 mmol) NaBH(OAc)₃ and 0.43 mL (7.5 mmol) AcOH in 100 mL 1,2-Dichloroethane.

HPLC (method A) t_{R}=5.76 min

TLC, Rf (CH₂Cl₂/MeOH 19:1)=0.33

MS (method D): 679 [M+]

Step 9

### (S)-9-{2-[((1R,2S)-1-Amino-2-vinyl-cyclopropanecarbonyl)-sulfamoyl]-phenylamino}-2-cyclopentyloxycarbonylamino-nonanoic acid methyl ester

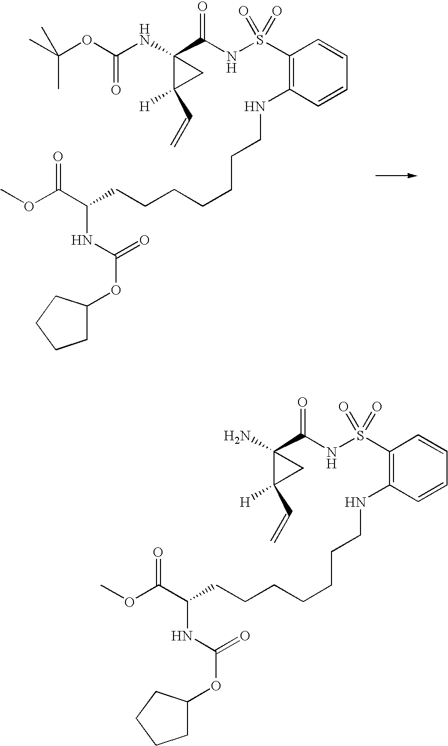

The title compound is prepared analogously as described for the title compound in Example 11 (step 4) using 310 mg (0.46 mmol) (S)-9-{2-[((1R,2S)-1-tert-Butoxycarbonylamino-2-vinyl-cyclopropane-carbonyl)-sulfamoyl]-phenylamino}-2-cyclopentyloxycarbonylamino-nonanoic acid methyl ester and 1 mL TFA in 10 mL DCM.

HPLC (method A) t_{R}=4.27 min

TLC, Rf (CH₂Cl₂/MeOH 9:1)=0.59

MS (method D): 579 [M+]

Step 10

### (2S,4R)-2-{(1R,2S)-1-[2-(S)-8-Cyclopentyloxycarbonylamino-8-methoxycarbonyl-octylamino)-benzenesulfonylaminocarbonyl]-2-vinyl-cyclopropylcarbamoyl}-4-[2-(2-isopropyl-amino-thiazol-4-yl)-7-methoxy-quinolin-4-yloxy]-pyrrolidine-1-carboxylic acid tert-butyl ester

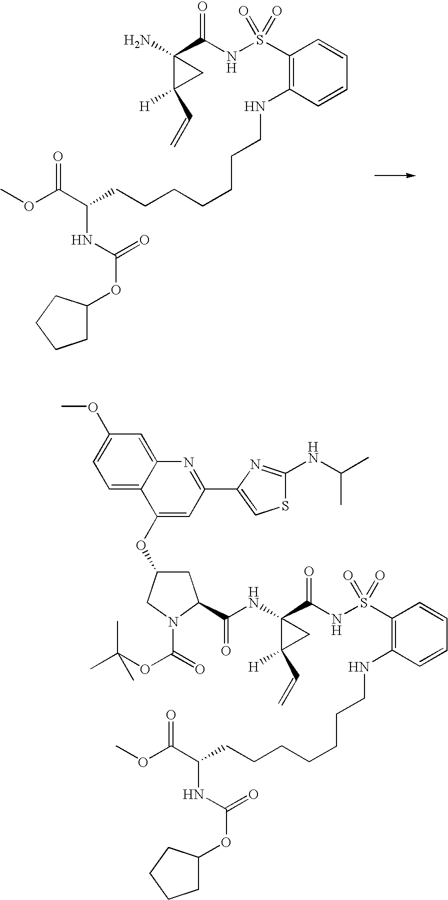

The title compound is prepared analogously as described for the title compound in Example 2 (step 1) using 185 mg (0.32 mmol) (S)-9-{2-[((1R,2S)-1-Amino-2-vinyl-cyclopropanecarbonyl)-sulfamoyl]-phenylamino}-2-cyclopentyloxycarbonylamino-nonanoic acid methyl ester, 170 mg (0.32 mmol) (2S,4R)-4-[2-(2-Isopropylamino-thiazol-4-yl)-7-methoxy-quinolin-4-yloxy]-pyrrolidine-1,2-dicarboxylic acid 1-tert-butyl ester, 183 mg (0.48 mmol) HATU and 124 mg (0.96 mmol) DIPEA in 10 mL DCM.

HPLC (method A) t_{R}=6.12 min

TLC, Rf (CH₂Cl₂/MeOH 19:1)=0.20

MS (method D): 1089 [M+]

Step 11

### (2S,4R)-2-{(1R,2S)-1-[2-((S)-8-Carboxy-8-cyclopentyloxycarbonylamino-octylamino)-benzenesulfonylaminocarbonyl]-2-vinyl-cyclopropylcarbamoyl}-4-[2-(2-isopropyl-amino-thiazol-4-yl)-7-methoxy-quinolin-4-yloxy]-pyrrolidine-1-carboxylic acid tert-butyl ester

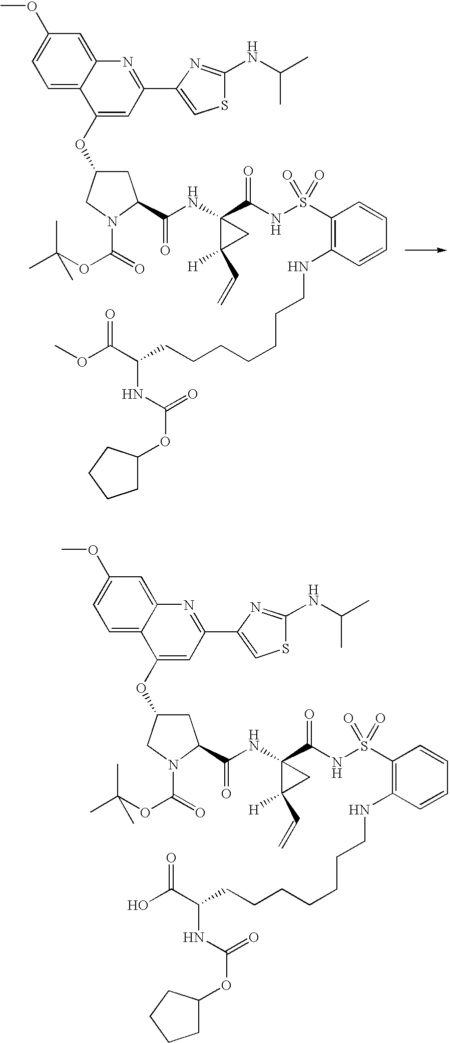

The title compound is prepared analogously as described for the title compound in Example 2 (step 2) using 205 mg (0.14 mmol) (2S,4R)-2-{(1R,2S)-1-[2-((S)-8-Cyclopentyloxycarbonylamino-8-methoxy-carbonyl-octylamino)-benzenesulfonylaminocarbonyl]-2-vinyl-cyclopropylcarbamoyl}-4-[2-(2-isopropyl-amino-thiazol-4-yl)-7-methoxy-quinolin-4-yloxyl-pyrrolidine-1-carboxylic acid tert-butyl ester (TFA-salt) and 59 mg (1.4 mmol) LiOH in 16 mL THF/MeOH/H₂O (2:1:1).

HPLC (method A) t_{R}=5.72 min

TLC, Rf (CH₂Cl₂/MeOH 9:1)=0.34

MS (method D): 1075 [M+]

Step 12

### (S)-2-Cyclopentyloxycarbonylamino-9-(2-{1(1R,2S)-1-({(2S,4R)-4-[2-(2-isopropylamino-thiazol-4-yl)-7-methoxy-quinolin-4-yloxy]-pyrrolidine-2-carbonyl}-amino)-2-vinyl-cyclopropanecarbonyl]-sulfamoyl}-phenylamino)-nonanoic acid

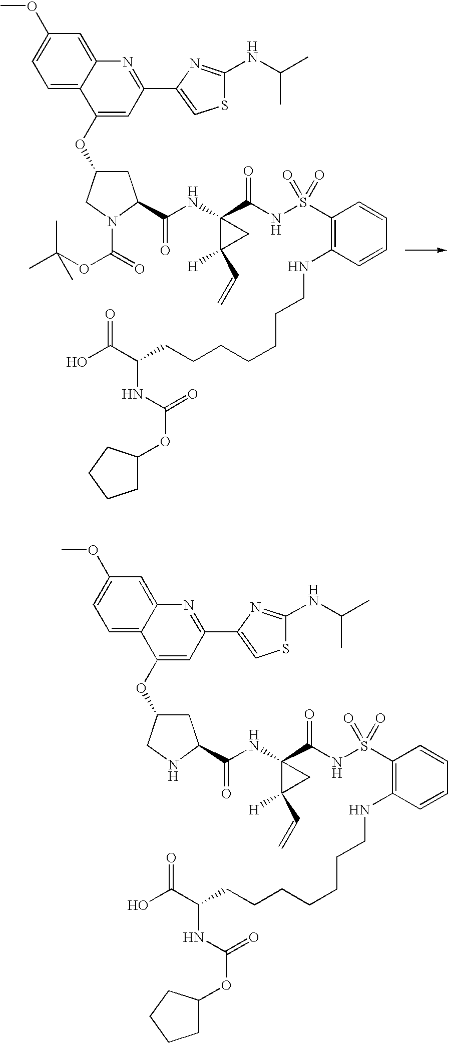

The title compound is prepared analogously as described for the title compound in Example 2 (step 3) using 119 mg (0.09 mmol) (2S,4R)-2-{(1R,2S)-1-[2-((S)-8-Carboxy-8-cyclopentyloxycarbonylamino-octylamino)-benzenesulfonylaminocarbonyl]-2-vinyl-cyclopropylcarbamoyl}-4-[2-(2-isopropyl-amino-thiazol-4-yl)-7-methoxy-quinolin-4-yloxy]-pyrrolidine-1-carboxylic acid tert-butyl ester (TFA-salt) and 0.5 mL TFA in 5 mL DCM.

HPLC (method A) t_{R}=5.33 min

TLC, Rf (CH₂Cl₂/MeOH/H₂O/AcOH 90:10:1:0.5)=0.46

MS (method D): 975 [M+]

### Example 22

### 4-Fluoro-1,3-dihydro-isoindole-2-carboxylic acid (8S,10R,14S)-14-cyclopentyloxycarbonyl-amino-5-[(1R,2S)-1-carbonylamino-2-vinyl-cyclopropyl]-2,2,4,7,13-pentaoxo-2λ*6*-thia-3,6,12,22-tetraaza-tricyclo[21.4.0.0*8,12*]heptacosa-1(23),24,26-trien-10-yl ester

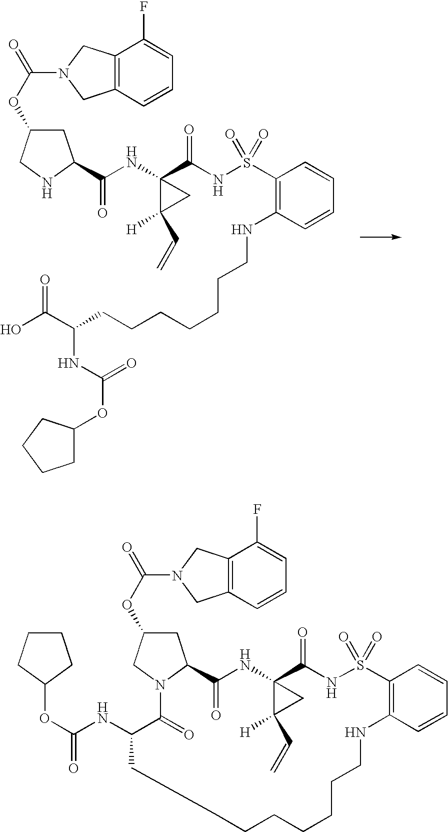

The title compound is prepared analogously as described for the title compound in Example 2 using 217 mg (0.21 mmol) 4-Fluoro-1,3-dihydro-isoindole-2-carboxylic acid (3R,5S)-5-{(1R,2S)-1-[2-(S)-8-carboxy-8-cyclopentyloxycarbonylamino-octylamino)-benzenesulfonylaminocarbonyl]-2-vinyl-cyclo-propylcarbamoyl}-pyrrolidin-3-yl ester (TFA-salt), 262 mg (2.0 mmol) DIPEA and 386 mg (1.1 mmol) HATU in 50 mL DCM and 1 mL DMF.

HPLC (method A) t_{R}=5.97 min

TLC, Rf (CH₂Cl₂/MeOH 9:1)=0.75

MS (method D): 823 [M+]

### Preparation of 4-Fluoro-1,3-dihydro-isoindole-2-carboxylic acid (3R,5S)-5-{(1R,2S)-1-[2-((S)-8-carboxy-8-cyclopentyloxycarbonylamino-octylamino)-benzenesulfonylaminocarbonyl]-2-vinyl-cyclopropylcarbamoyl}-pyrrolidin-3-yl ester

Step 1

### 4-Fluoro-1,3-dihydro-isoindole-2-carboxylic acid (3R,5S)-1-tert-butoxycarbonyl-5-{(1R,2S)-1-[2-((S)-8-cyclopentyloxycarbonylamino-8-methoxycarbonyl-octylamino)-benzenesulfonylamino-carbonyl]-2-vinyl-cyclopropylcarbamoyl}-pyrrolidin-3-yl ester

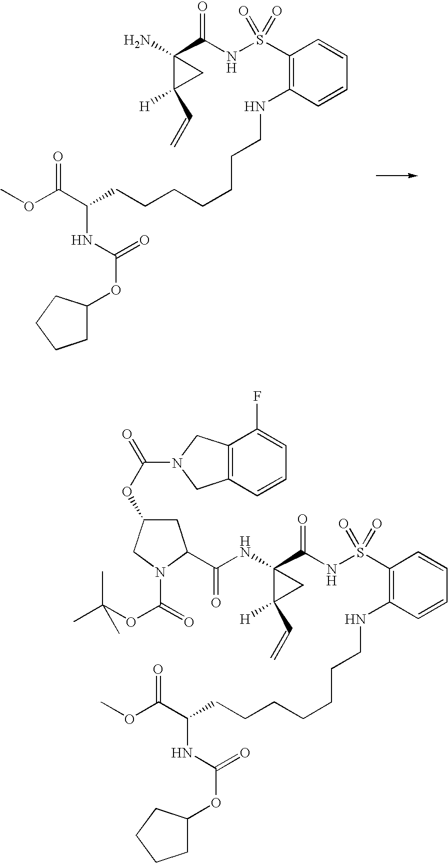

The title compound is prepared analogously as described for the title compound in Example 2 (step 1) using 368 mg (0.46 mmol) (S)-9-{2-[((1R,2S)-1-Amino-2-vinyl-cyclopropanecarbonyl)-sulfamoyl]-phenylamino}-2-cyclopentyloxycarbonylamino-nonanoic acid methyl ester (TFA-salt), 216 mg (0.55 mmol) (2S,4R)-4-(4-Fluoro-1,3-dihydro-isoindole-2-carbonyloxy)-pyrrolidine-1,2-dicarboxylic acid 1-tert-butyl ester, 260 mg (0.68 mmol) HATU and 354 mg (2.74 mmol) DIPEA in 8 mL DCM.

HPLC (method A) t_{R}=6.15 min

TLC, Rf (CH₂Cl₂/MeOH 19:1)=0.50

MS (method D): 955 [M+]

Step 2

### 4-Fluoro-1,3-dihydro-isoindole-2-carboxylic acid (3R,5S)-1-tert-butoxycarbonyl-5-{(1R,2S)-1-[2-((S)-8-carboxy-8-cyclopentyloxycarbonylamino-octylamino)-benzenesulfonylaminocarbonyl]-2-vinyl-cyclopropylcarbamoyl}-pyrrolidin-3-yl ester

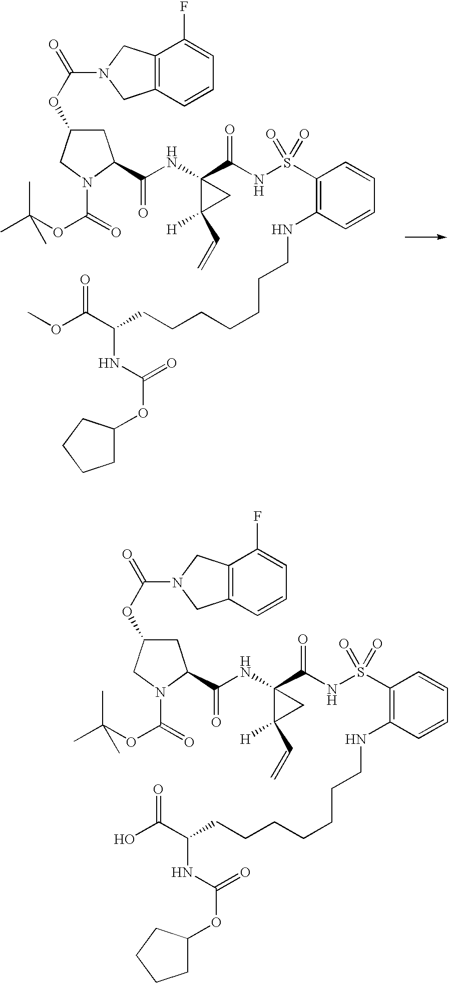

The title compound is prepared analogously as described for the title compound in Example 2 (step 2) using 217 mg (0.20 mmol) 4-Fluoro-1,3-dihydro-isoindole-2-carboxylic acid (3R,5S)-1-tert-butoxycarbonyl-5-{(1R,2S)-1-[2-((S)-8-cyclopentyloxycarbonylamino-8-methoxycarbonyl-octylamino)-benzenesulfonylamino-carbonyl]-2-vinyl-cyclopropylcarbamoyl}-pyrrolidin-3-yl ester (TFA-salt) and 49 mg (2.0 mmol) LiOH in 16 mL THF/MeOH/H₂O (2:1:1).

HPLC (method A) t_{R}=5.59 min

TLC, Rf (CH₂Cl₂/MeOH 9:1)=0.60

MS (method D): 941 [M+]

Step 3

### 4-Fluoro-1,3-dihydro-isoindole-2-carboxylic acid (3R,5S)-5-{(1R,2S)-1-[2-((S)-8-carboxy-8-cyclopentyloxycarbonylamino-octylamino)-benzenesulfonylaminocarbonyl]-2-vinyl-cyclopropylcarbamoyl}-pyrrolidin-3-yl ester

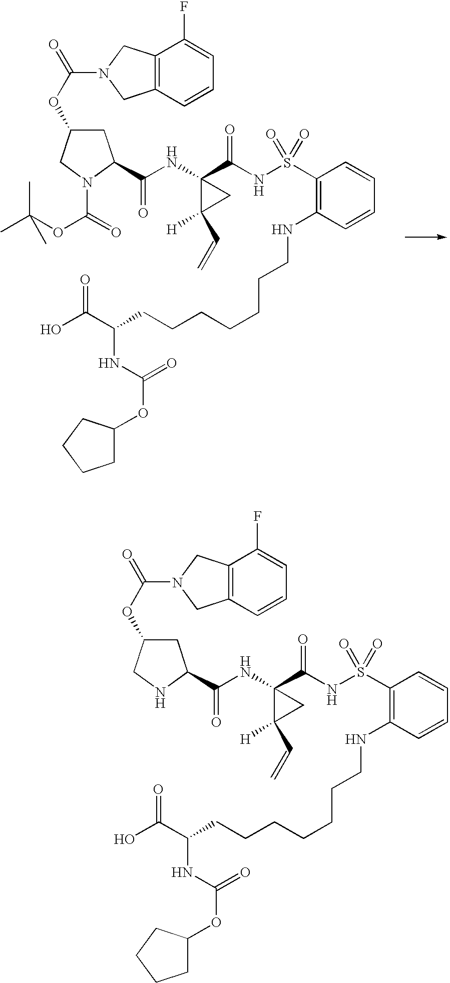

The title compound is prepared analogously as described for the title compound in Example 2 (step 3) using 191 mg (0.20 mmol) 4-Fluoro-1,3-dihydro-isoindole-2-carboxylic acid (3R,5S)-1-tert-butoxycarbonyl-5-{(1R,2S)-1-[2-((S)-8-carboxy-8-cyclopentyloxycarbonylamino-octylamino)-benzene-sulfonylaminocarbonyl]-2-vinyl-cyclopropylcarbamoyl}-pyrrolidin-3-yl ester and 1 mL TFA in 25 mL DCM.

HPLC (method A) t_{R}=5.01 min

MS (method D): 841 [M+]

### Example 23

### {(8S,10R,14S)-10-[2-(2-Isopropylamino-thiazol-4-yl)-7-methoxy-quinolin-4-yloxy]-5-[(1R,2S)-1-carbonylamino-2-vinyl-cyclopropyl]-2,2,4,7,13,21-hexaoxo-2λ*6*-thia-3,6,12,22-tetraaza-tricyclo[21.4.0.0*8,12*]heptacosa-1(23),24,26-trien-14-yl}-carbamic acid cyclopentyl ester

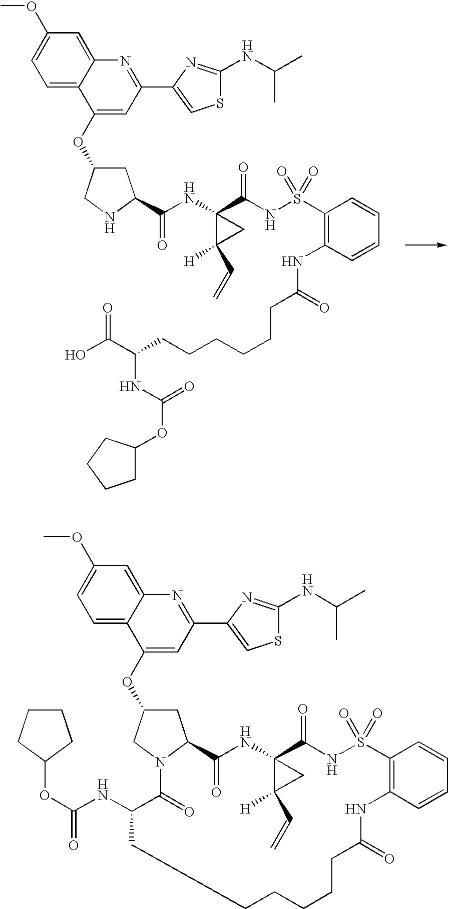

The title compound is prepared analogously as described for the title compound in Example 2 using 118 mg (0.097 mmol) (S)-2-Cyclopentyloxycarbonylamino-8-(2-{[(1R,2S)-1-({(2S,4R)-4-[2-(2-isopropyl-amino-thiazol-4-yl)-7-methoxy-quinolin-4-yloxy]-pyrrolidine-2- carbonyl}-amino)-2-vinyl-cyclo-propane-carbonyl]-sulfamoyl}-phenylcarbamoyl)-octanoic acid (TFA-salt), 126 mg (0.97 mmol) DIPEA and 184 mg (0.49 mmol) HATU in 100 mL DCM and 2 mL DMF.

HPLC (method A) t_{R}=5.43 min

TLC, Rf (CH₂Cl₂/MeOH 9:1)=0.45

MS (method D): 971 [M+]

### Preparation of (S)-2-Cyclopentyloxycarbonylamino-8-(2-{[(1R,2S)-1-({(2S,4R)-4-[2-(2-isopropylamino-thiazol-4-yl)-7-methoxy-quinolin-4-yloxy]-pyrrolidine-2-carbonyl}-amino)-2-vinyl-cyclo-propanecarbonyl]-sulfamoyl}-phenylcarbamoyl)-octanoic acid

Step 1

### (S)-2-Cyclopentyloxycarbonylamino-nonanedioic acid 1-methyl ester

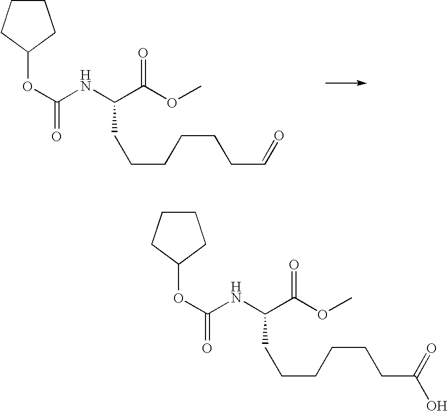

To a solution of 1.88 g (6.0 mmol) ((S)-2-Cyclopentyloxycarbonylamino-9-oxo-nonanoic acid methyl ester in 20 mL tBuOH is added at rt 2.1 g (30 mmol) 2-Methyl-2-buten, 2.81 g (18 mmol) NaH₂PO₄ (in 15 mL H₂O) and 1.62 g (18 mmol) NaClO₂ (in 15 mL H₂O). After stirring for 1 h, the solvent is removed in vacuo, the residue is diluted with water, acidified with 0.5 N HCl and extracted with EtOAc. The combined organic phase is dried with Na₂SO₄, filtered and the solvent is removed in vacuo. The residue is purified by FC on silica (eluent: CH₂Cl₂/MeOH 98:2→95:5) to give the title compound as a colorless oil.

HPLC (method A) t_{R}=3.83 min

TLC, Rf (CH₂Cl₂/MeOH 19:1)=0.26

MS (method D): 330 [M+H]

Step 2

### (S)-8-{2-[((1 R,2S)-1-tert-Butoxycarbonylamino-2-vinyl-cyclopropanecarbonyl)-sulfamoyl]-phenylcarbamoyl}-2-cyclopentyloxycarbonylamino-octanoic acid methyl ester

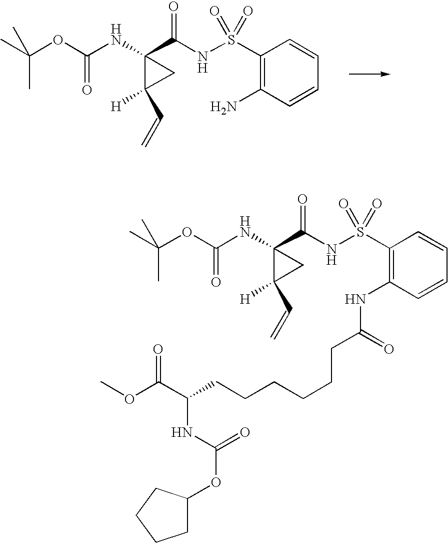

The title compound is prepared analogously as described for the title compound in Example 1 (Step 2) using 0.85 g (2.23 mmol) [(R1R,2S)-1-(2-Amino-benzenesulfonylaminocarbonyl)-2-vinyl-cyclopropyl]-carbamic acid tert-butyl ester, 0.96 g (2.9 mmol) (S)-2-Cyclopentyloxycarbonylamino-nonanedioic acid 1-methyl ester, 0.40 g (3.3 mmol) Benzotriazole, 0.40 g (3.3 mmol) Thionylchloride, 0.92 g (10 mmol) NEt₃ and 100 mg DMAP in 50 mL DCM.

HPLC (method A) t_{R}=5.31 min

TLC, Rf (CH₂Cl₂/MeOH 19:1)=0.31

MS (method D): 693 [M+]

Step 3

### (S)-8-{2-[((1R,2S)-1-Amino-2-vinyl-cyclopropanecarbonyl)-sulfamoyl]-phenylcarbamoyl}-2-cyclopentyloxycarbonylamino-octanoic acid methyl ester

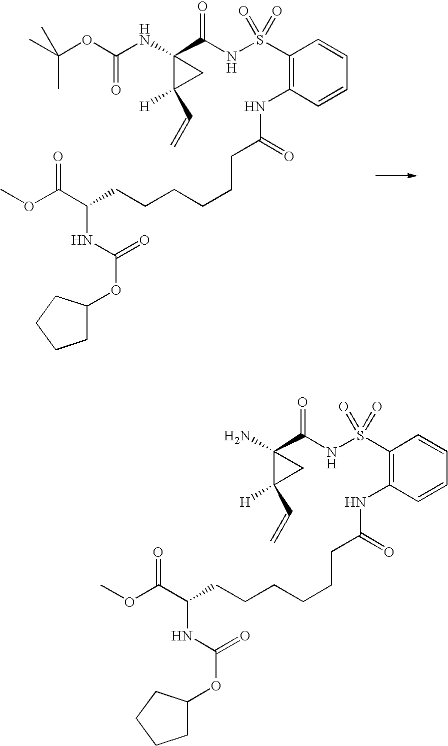

The title compound is prepared analogously as described for the title compound in Example 1 (Step 3) using 0.85 g (2.23 mmol) (S)-8-{2-[((1R,2S)-1-tert-Butoxycarbonylamino-2-cyclopropane-carbonyl)-sulfamoyl]-phenylcarbamoyl}-2-cyclopentyloxycarbonylamino-octanoic acid methyl ester and 5 mL 4N HCl in Dioxane.

HPLC (method A) t_{R}=3.76 min

MS (method D): 593 [M+]

Step 4

### (2S,4R)-2-{(1R,2S)-1-[2-((S)-8-Cyclopentyloxycarbonylamino-8-methoxycarbonyl-octanoyl-amino)-benzenesulfonylaminocarbonyl]-2-vinyl-cyclopropylcarbamoyl}-4-(-[2-(2-isopropyl-amino-thiazol-4-yl)-7-methoxy-quinolin-4-yloxy]pyrrolidine-1-carboxylic acid tert-butyl ester

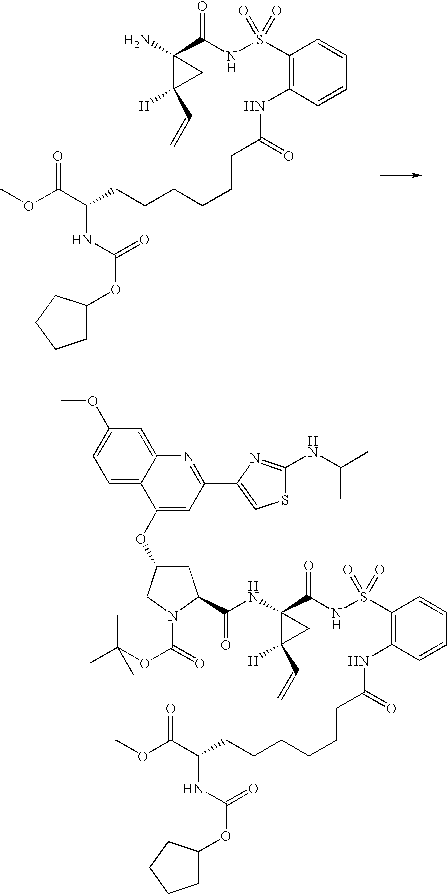

The title compound is prepared analogously as described for the title compound in Example 2 (step 1) using 190 mg (0.27 mmol) (S)-8-{2-[((1R,2S)-1-Amino-2-vinyl-cyclopropanecarbonyl)-sulfamoyl]-phenylcarbamoyl}-2-cyclopentyloxycarbonylamino-octanoic acid methyl ester (HCl-salt), 141 mg (0.27 mmol) (2S,4R)-4-[2-(2-Isopropylamino-thiazol-4-yl)-7-methoxy-quinolin-4-yloxy]-pyrrolidine-1,2-dicarboxylic acid 1-tert-butyl ester, 152 mg (0.40 mmol) HATU and 103 mg (0.80 mmol) DIPEA in 10 mL DCM.

HPLC (method A) t_{R}=5.63 min

TLC, Rf (CH₂Cl₂/MeOH 9:1)=0.20

MS (method D): 1103 [M+]

Step 5

### (2S,4R)-2-{(1R,2S)-1-[2-((S)-8-Carboxy-8-cyclopentyloxycarbonylamino-octanoylamino)-benzenesulfonylaminocarbonyl]-2-vinyl-cyclopropylcarbamoyl}-4-(-[2-(2-isopropyl-amino-thiazol-4-yl)-7-methoxy-quinolin-4-yloxy]-pyrrolidine-1-carboxylic acid tert-butyl ester

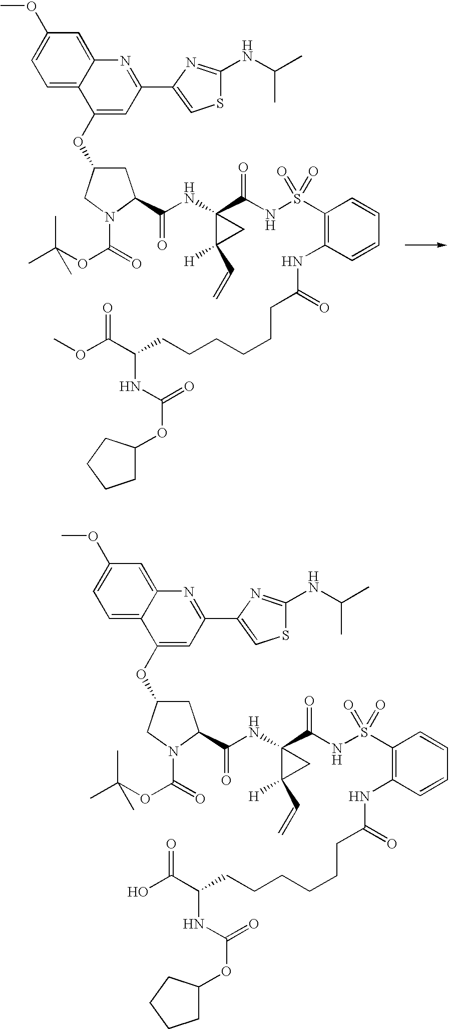

The title compound is prepared analogously as described for the title compound in Example 2 (step 2) using 120 mg (0.099 mmol) (2S,4R)-2-{(1R,2S)-1-[2-((S)-8-Cyclopentyloxycarbonylamino-8-methoxycarbonyl-octanoyl-amino)-benzenesulfonylaminocarbonyl]-2-vinyl-cyclopropylcarbamoyl}-4-(-[2-(2-isopropyl-amino-thiazol-4-yl)-7-methoxy-quinolin-4-yloxy]-pyrrolidine-1-carboxylic acid tert-butyl ester (TFA-salt) and 42 mg (0.99 mmol) LiOH in 16 mL THF/MeOH/H₂O (2:1:1).

HPLC (method A) t_{R}=5.38 min

TLC, Rf (CH₂Cl₂/MeOH 9:1)=0.11

MS (method D): 1089 [M+]

Step 6

### (S)-2-Cyclopentyloxycarbonylamino-8-(2-{[(1R,2S)-1-({(2S,4R)-4-[2-(2-isopropylamino-thiazol-4-yl)-7-methoxy-quinolin-4-yloxy]-pyrrolidine-2-carbonyl}-amino)-2-vinyl-cyclo-propanecarbonyl]-sulfamoyl}-phenylcarbamoyl)-octanoic acid

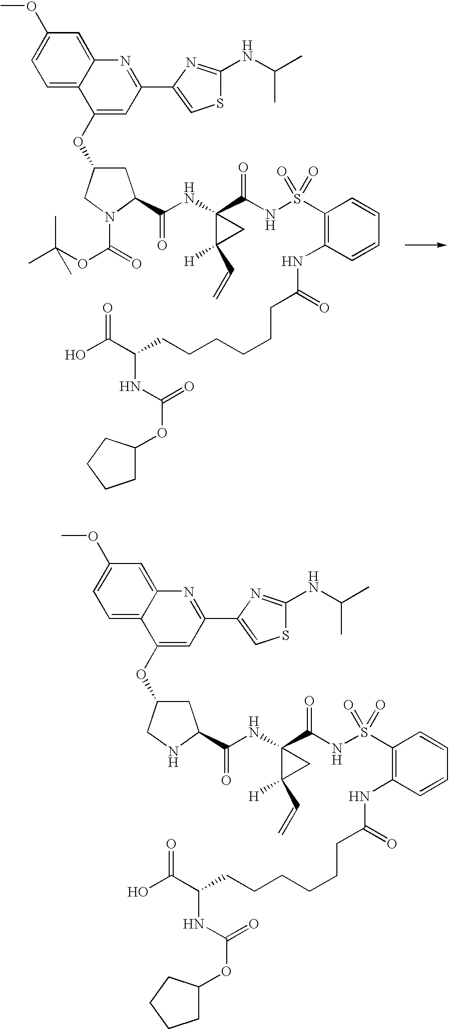

The title compound is prepared analogously as described for the title compound in Example 2 (step 3) using 106 mg (0.097 mmol) (2S,4R)-2-{(1R,2S)-1-[2-((S)-8-Carboxy-8-cyclopentyloxycarbonylamino-octanoylamino)-benzenesulfonylaminocarbonyl]-2-vinyl-cyclopropylcarbamoyl}-4-(4-(-[2-(2-isopropyl-amino-thiazol-4-yl)-7-methoxy-quinolin-4-yloxy]-pyrrolidine-1-carboxylic acid tert-butyl ester and 1 mL TFA in 5 mL DCM.

HPLC (method A) t_{R}=4.78 min

TLC, Rf (CH₂Cl₂/MeOH/H₂O/AcOH 90:10:1:0.5)=0.21

MS (method D): 989 [M+]

### Example 24

### (8S,10R,14S)-10-(5-Chloro-pyridin-2-yloxy)-5-[(1R,2S)-1-carbonylamino-2-vinyl-cyclopropyl]-2,2,4,7,13-pentaoxo-2λ*6*-thia-3,6,12,22-tetraaza-tricyclo[21.4.0.0*8,12*]heptacosa-1(23),24,26-trien-14-yl]-carbamic acid cyclopentyl ester

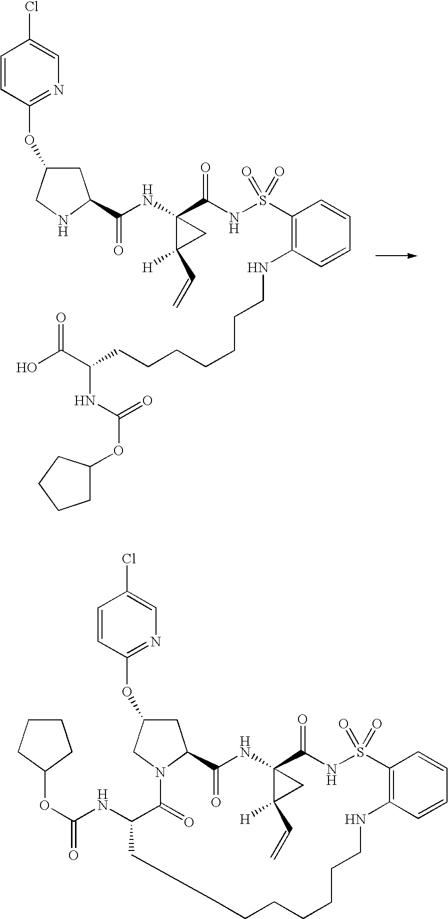

The title compound is prepared analogously as described for the title compound in Example 2 using 250 mg (0.25 mmol) (S)-9-{2-[((1R,2S)-1-{[(2S,4R)-4-(5-Chloro-pyridin-2-yloxy)-pyrrolidine-2-carbonyl]amino}-2-vinyl-cyclopropanecarbonyl)-sulfamoyl]-phenylamino}-2-cyclopentyloxycarbonyl-amino-nonanoic acid (TFA-salt), 3.18 mg (2.5 mmol) DIPEA and 468 mg (1.2 mmol) HATU in 50 mL DCM and 1 mL DMF.

HPLC (method A) t_{R}=6.27 min

TLC, Rf (CH₂Cl₂/MeOH 19:1)=0.37

MS (method D): 771 [M+]

### Preparation of (S)-9-{2-[((1R,2S)-1-{[(2S,4R)-4-(5-Chloro-pyridin-2-yloxy)-pyrrolidine-2-carbonyl]-amino}-2-vinyl-cyclopropanecarbonyl)-sulfamoyl]-phenylamino}-2-cyclopentyloxy-carbonyl-amino-nonanoic acid

Step 1

### (2S,4R)-4-(5-Chloro-pyridin-2-yloxy)-2-{(1R,2S)-1-[2-((S)-8-cyclopentyloxycarbonylamino-8-methoxycarbonyl-octylamino)-benzenesulfonylaminocarbonyl]-2-vinyl-cyclopropylcarbamoyl}-pyrrolidine-1-carboxylic acid tert-butyl ester

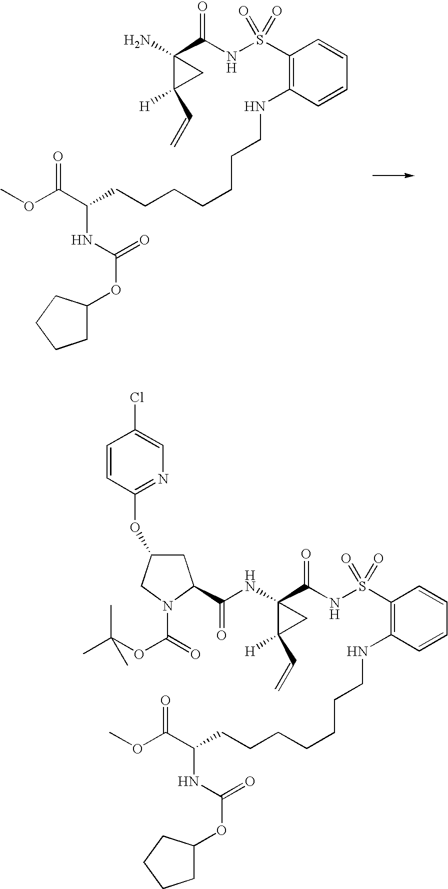

The title compound is prepared analogously as described for the title compound in Example 2 (step 1) using 380 mg (0.47 mmol) (S)-9-{2-[((1R,2S)-1-Amino-2-vinyl-cyclopropanecarbonyl)-sulfamoyl]-phenylamino}-2-cyclopentyloxycarbonylamino-nonanoic acid methyl ester (TFA-salt), 194 mg (0.57 mmol) ((2S,4R)-4-(5-Chloro-pyridin-2-yloxy)-pyrrolidine-1,2-dicarboxylic acid 1-tert-butyl ester (prepared according to WO 2005035525), 269 mg (0.70 mmol) HATU and 365 mg (2.82 mmol) DIPEA in 10 mL DCM.

HPLC (method A) t_{R}=6.33 min

TLC, Rf (CH₂Cl₂/MeOH 9:1)=0.69

MS (method D): 903 [M+]

Step 2

### (2S,4R)-2-{(1R,2S)-1-[2-((S)-8-Carboxy-8-cyclopentyloxycarbonylamino-octylamino)-benzene-sulfonylaminocarbonyl]-2-vinyl-cyclopropylcarbamoyl}-4-(5-chloro-pyridin-2-yloxy)-pyrrolidine-1-carboxylic acid tert-butyl ester

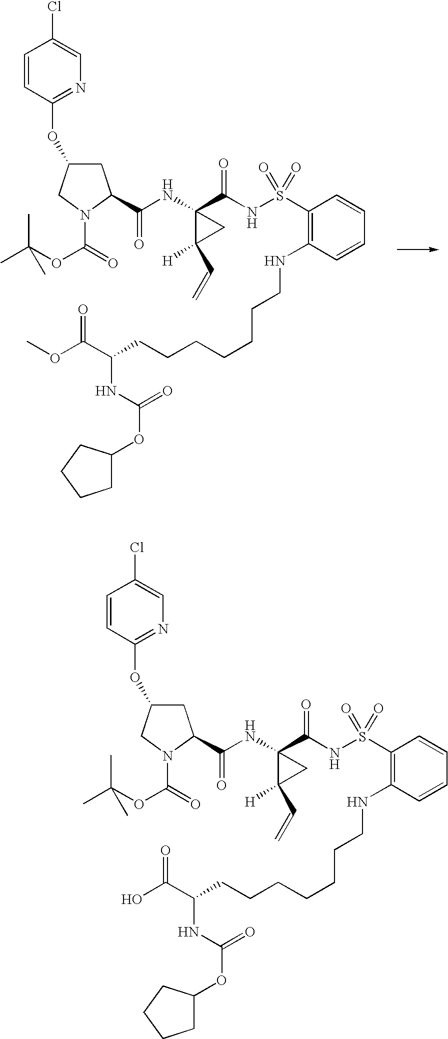

The title compound is prepared analogously as described for the title compound in Example 2 (step 2) using 250 mg (0.25 mmol) (2S,4R)-4-(5-Chloro-pyridin-2-yloxy)-2-{(1R, 2S)-1-[2-((S)-8-cyclopentyloxy-carbonylamino-8-methoxycarbonyl-octylamino)-benzenesulfonylaminocarbony]-2-vinyl-cyclopropyl-carbamoyl}-pyrrolidine-1-carboxylic acid tert-butyl ester (TFA-salt) and 59 mg (2.5 mmol) LiOH in 16 mL THF/MeOH/H₂O (2:1:1).

HPLC (method A) t_{R}=5.86 min

TLC, Rf (CH₂Cl₂/MeOH 9:1)=0.48

MS (method D): 889 [M+]

Step 3

### (S)-9-{2-[((1R,2S)-1-{[(2S,4R)-4-(5-Chloro-pyridin-2-yloxy)-pyrrolidine-2-carbonyl]-amino}-2-vinyl-cyclopropanecarbonyl)-sulfamoyl]-phenylamino}-2-cyclopentyloxycarbonylamino-nonanoic acid

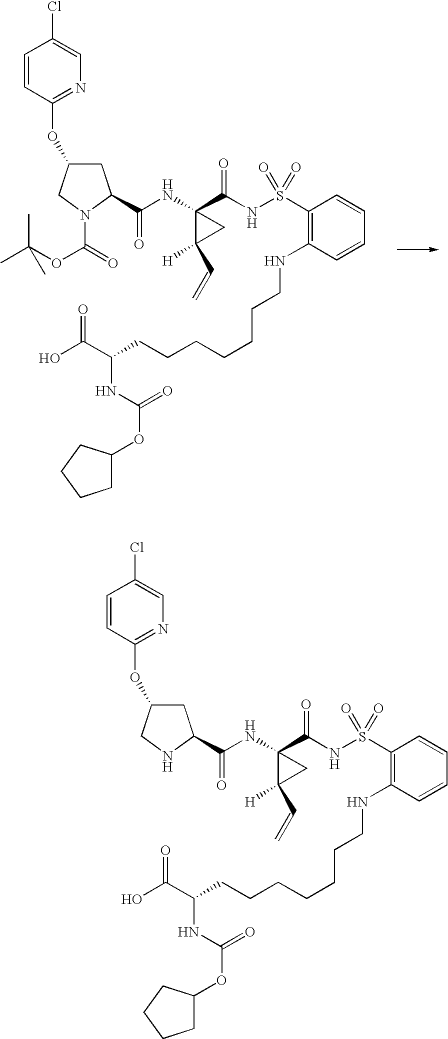

The title compound is prepared analogously as described for the title compound in Example 2 (step 3) using 219 mg (0.25 mmol) (2S,4R)-2-{(1R,2S)-1-[2-((S)-8-Carboxy-8-cyclopentyloxycarbonylamino-octylamino)-benzene-sulfonylaminocarbonyl]-2-vinyl-cyclopropylcarbamoyl}-4-(5-chloro-pyridin-2-yloxy)-pyrrolidine-1-carboxylic acid tert-butyl ester and 1 mL TFA in 10 mL DCM.

HPLC (method A) t_{R}=4.99 min

TLC, Rf (CH₂Cl₂/MeOH 9:1)=0.51

MS (method D): 789 [M+]

### Example 25

### ((8S,14S)-5-[(1R,2S)-1-carbonylamino-2-vinyl-cyclopropyl]-2,2,4,7,13-pentaoxo-2λ*6*-thia-3,6,12,22-tetraaza-tricyclo[21.4.0.0*8,12*]heptacosa-1(23),24,26-trien-14-yl)-carbamic acid cyclopentyl ester

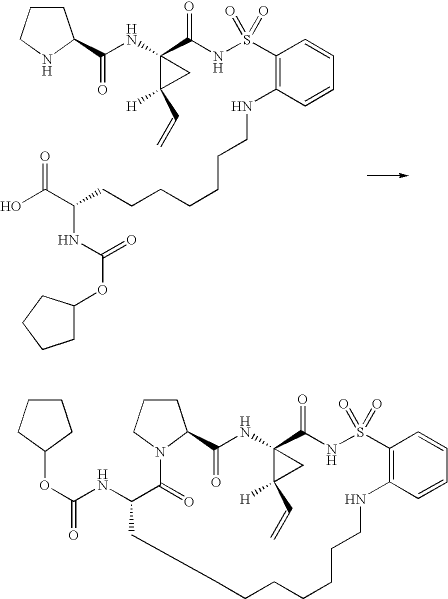

The title compound is prepared analogously as described for the title compound in Example 2 using 62 mg (0.07 mmol) ((S)-2-Cyclopentyloxycarbonylamino-9-[2-({(1R,2S)-1-[((S)-pyrrolidine-2-carbonyl)-amino]-2-vinyl-cyclopropanecarbonyl}-sulfamoyl)-phenylamino]-nonanoic acid (TFA-salt), 90 mg (0.7 mmol) DIPEA and 133 mg (0.35 mmol) HATU in 25 mL DCM and 0.5 mL DMF.

HPLC (method A) t_{R}=5.68 min

TLC, Rf (CH₂Cl₂/MeOH 19:1)=0.41

MS (method D): 644 [M+]

### Preparation of ((S)-2-Cyclopentyloxycarbonylamino-9-[2-({(1R,2S)-1-[((S)-pyrrolidine-2-carbonyl)-amino]-2-vinyl-cyclopropanecarbonyl}-sulfamoyl)-phenylamino]-nonanoic acid

Step 1

### (S)-2-{(1R,2S)-1-[2-((S)-8-Cyclopentyloxycarbonylamino-8-methoxycarbonyl-octylamino)-benzenesulfonylaminocarbonyl]-2-vinyl-cyclopropylcarbamoyl}-pyrrolidine-1-carboxylic acid tert-butyl ester

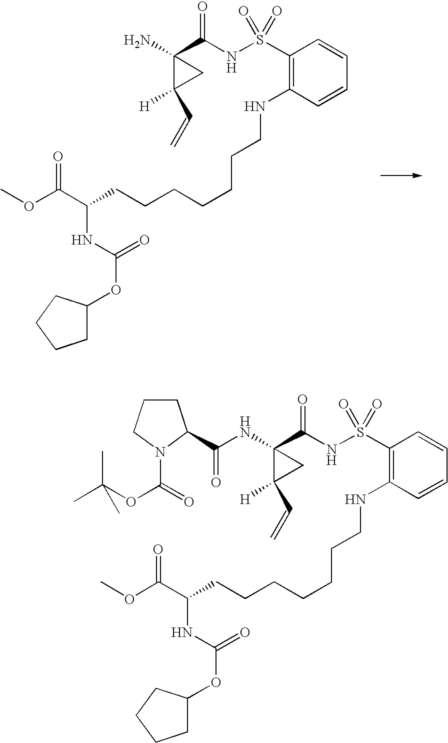

The title compound is prepared analogously as described for the title compound in Example 2 (step 1) using 150 mg (0.19 mmol) (S)-9-{2-[((1R,2S)-1-Amino-2-vinyl-cyclopropanecarbonyl)-sulfamoyl}-phenylamino}-2-cyclopentyloxycarbonylamino-nonanoic acid methyl ester (TFA-salt), 48 mg (0.22 mmol) (S)-Pyrrolidine-1,2-dicarboxylic acid 1-tert-butyl ester, 106 mg (0.28 mmol) HATU and 144 mg (1.1 mmol) DIPEA in 10 mL DCM.

HPLC (method A) t_{R}=5.84 min

TLC, Rf (CH₂Cl₂/MeOH 19:1)=0.63

MS (method D): 776 [M+]

Step 2

### (S)-2-{(1R,2S)-1-[2-((S)-8-Carboxy-8-cyclopentyloxycarbonylamino-octylamino)-benzenesulfonylaminocarbonyl]-2-vinyl-cyclopropylcarbamoyl}-pyrrolidine-1-carboxylic acid tert-butyl ester

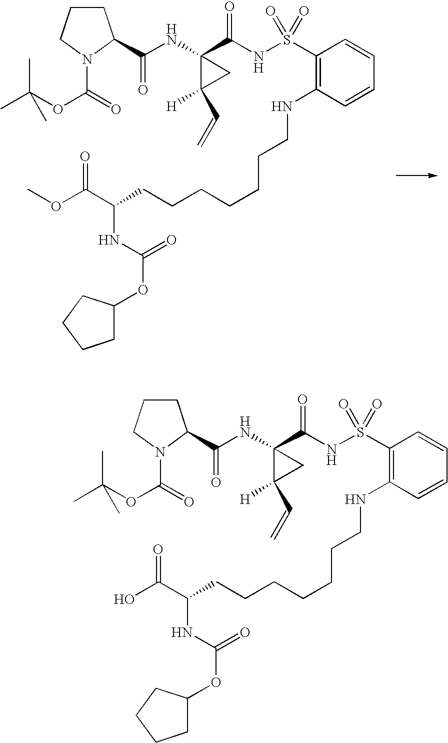

The title compound is prepared analogously as described for the title compound in Example 2 (step 2) using 79 mg (0.09 mmol) (S)-2-{(1R,2S)-1-[2(S)-8-Cyclopentyloxycarbonylamino-8-methoxycarbonyl-octylamino)-benzenesulfonylaminocarbonyl]-2-vinyl-cyclopropylcarbamoyl}-pyrrolidine-1-carboxylic acid tert-butyl ester (TFA-salt) and 21 mg (0.89 mmol) LiOH in 16 mL THF/MeOH/H₂O (2:1:1).

HPLC (method A) t_{R}=5.35 min

TLC, Rf (CH₂Cl₂/MeOH 9:1)=0.37

MS (method D): 762 [M+]

Step 3

### (S)-2-Cyclopentyloxycarbonylamino-9-[2-({(1R,2S)-1-[((S)-pyrrolidine-2-carbonyl)-amino]-2-vinyl-cyclopropanecarbonyl}-sulfamoyl)-phenylamino]-nonanoic acid

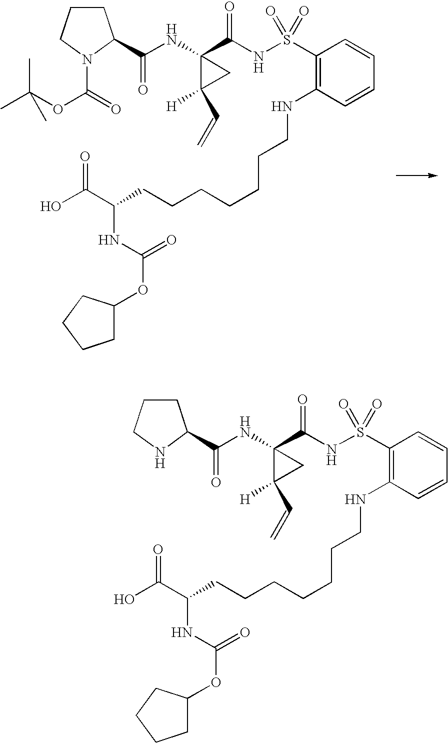

The title compound is prepared analogously as described for the title compound in Example 2 (step 3) using 59 mg (0.08 mmol) (S)-2-{(1R,2S)-1-[2-((S)-8-Carboxy-8-cyclopentyloxycarbonylamino-octylamino)-benzenesulfonylaminocarbonyl]-2-vinyl-cyclopropylcarbamoyl}-pyrrolidine-1-carboxylic acid tert-butyl ester and 1 mL TFA in 10 mL DCM.

HPLC (method A) t_{R}=4.44 min

TLC, Rf (CH₂Cl₂/MeOH 9:1)=0.40

MS (method D): 662 [M+]

### Example 26

### cyclopentyl [(1R,2S,13′S)-23′,23′-dioxido-14′,18′,21′-trioxo-2-vinyl-5′,6′,7′,8′,9′,10′,11′,12′,13′,14′,16a′,17′,17a′,17b′,18′,19′,21′, 22′-octadecahydro-16′-H-spiro[cyclopropane-1,20′-cyclopropa[3,4]pyrrolo[2,1-g]1,2,5,8,18]benzothiatetraazacycloicosin]-13′-yl]carbamate

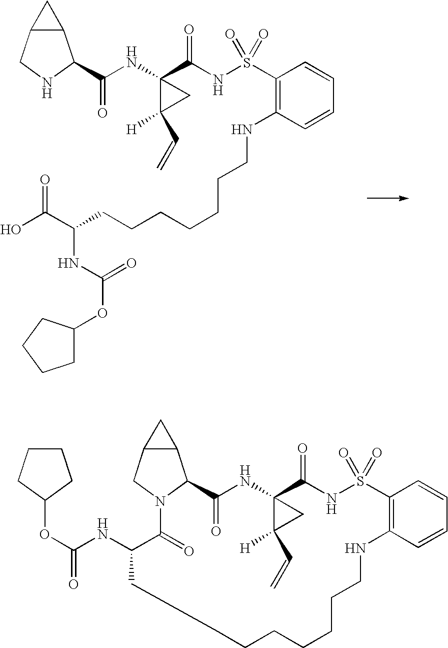

The title compound is prepared analogously as described for the title compound in Example 2 using 176 mg (0.20 mmol) (S)-9-[2-({(1R,2S)-1-[(3-Aza-bicyclo[3.1.0]hexane-2-carbonyl)-amino]-2-vinyl-cyclopropane-carbonyl}-sulfamoyl)-phenylamino]-2-cyclopentyloxycarbonylamino-nonanoic acid (TFA-salt), 252 mg (0.98 mmol) DIPEA and 371 mg (1.95 mmol) HATU in 50 mL DCM and 1 mL DMF.

HPLC (method A) t_{R}=5.68 min

TLC, Rf (CH₂Cl₂/MeOH 9:1)=0.62

MS (method D): 656 [M+]

### Preparation of (S)-9-[2-({(1R,2S)-1-1(3-Aza-bicyclo[3.1.0]hexane-2-carbonyl)-amino]-2-vinyl--cyclopropane-carbonyl}-sulfamoyl)-phenylamino]-2-cyclopentyloxycarbonylamino-nonanoic acid

Step 1

### trans-rac-3-Aza-bicyclo[3.1.0]hexane-2,3-dicarboxylic acid 3-tert-butyl ester

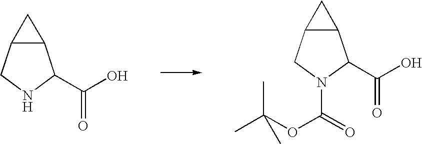

To a solution of 0.70 g (5.5. mmol) trans-rac-3-Aza-bicyclo[3.1.0]hexane-2-carboxylic acid (Aldrich) in 20 mL DCM is added 1.11 g (11.0 mmol) NEt₃. 1.68 g (7.7 mmol) (BOC₂)O is added in three portions over 10 min and the mixture is stirred overnight at ambient temperature. The reaction is quenched by addition of water, acidified with 1N HCl and extracted with DCM. The combined organic phase is washed with brine, dried with Na₂SO₄, filtered and the solvent is removed in vacuo. The residue is purified by FC on silica (eluent: CH₂Cl₂/MeOH 98:2) to give the title compound as a colorless solid.

TLC, Rf (CH₂Cl₂/MeOH 9:1)=0.62

MS (method D): 172 [M-55]

Step 2

### 2-{(1R,2S)-1-[2-((S)-8-Cyclopentyloxycarbonylamino-8-methoxycarbonyl-octylamino)-benzene-sulfonylaminocarbonyl]-2-vinyl-cyclopropylcarbamoyl}-3-aza-bicyclo[3.1.0]hexane-3-carboxylic acid tert-butyl ester

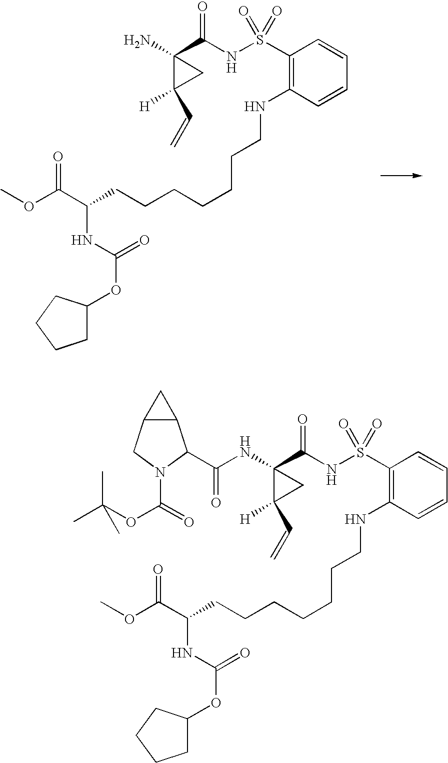

The title compound is prepared analogously as described for the title compound in Example 2 (step 1) using 244 mg (0.30 mmol) (S)-9-{2-[((1R,2S)-1-Amino-2-vinyl-cyclopropanecarbonyl)-sulfamoyl]-phenylamino}-2-cyclopentyloxycarbonylamino-nonanoic acid methyl ester (TFA-salt), 82 mg (0.36 mmol) trans-rac-3-Aza-bicyclo[3.1.0]hexane-2,3-dicarboxylic acid 3-tert-butyl ester, 172 mg (0.45 mmol) HATU and 234 mg (1.8 mmol) DIPEA in 10 mL DCM.

HPLC (method A) t_{R}=5.90 min

TLC, Rf (CH₂Cl₂/MeOH 9:1)=0.69

MS (method D): 788 [M+]

Step 3

### 2-{(1R,2S)-1-[2-((S)-8-Carboxy-8-cyclopentyloxycarbonylamino-octylamino)-benzenesulfonyl-aminocarbonyl]-2-vinyl-cyclopropylcarbamoyl}-3-aza-bicyclo[3.1.0]hexane-3-carboxylic acid tert-butyl ester

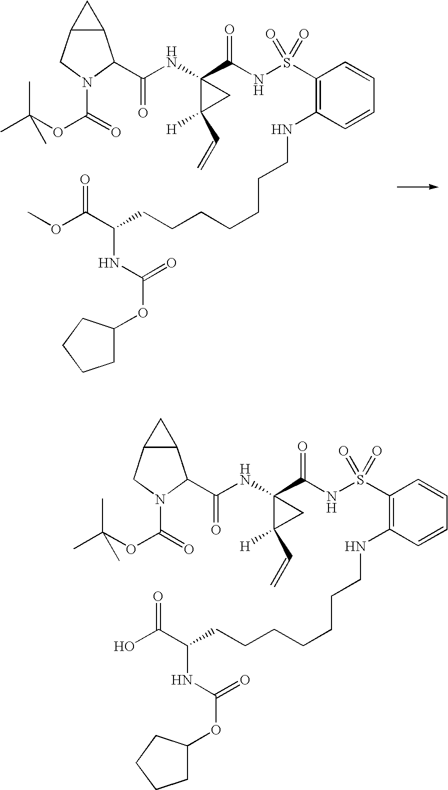

The title compound is prepared analogously as described for the title compound in Example 2 (step 2) using 183 mg (0.20 mmol) 2-{(1R,2S)-1-[2-((S)-8-Cyclopentyloxycarbonylamino-8-methoxycarbonyl-octylamino)-benzene-sulfonylaminocarbonyl]-2-vinyl-cyclopropylcarbamoyl}-3-aza-bicyclo[3.1.0]hexane-3-carboxylic acid tert-butyl ester (TFA-salt) and 49 mg (2.0 mmol) LiOH in 20 mL THF/MeOH/H₂O (2:1:1).

HPLC (method A) t_{R}=5.42 min

TLC, Rf (CH₂Cl₂/MeOH 9:1)=0.50

MS (method D): 774 [M+]

Step 4

### (S)-9-[2-({(1R,2S)-1-[(3-Aza-bicyclo[3.1.0]hexane-2-carbonyl)-amino]-2-vinyl-cyclopropane-carbonyl}-sulfamoyl)-phenylamino]-2-cyclopentyloxycarbonylamino-nonanoic acid

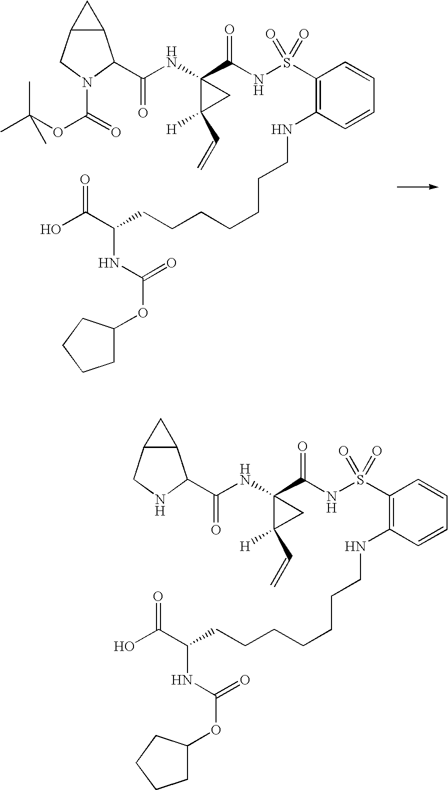

The title compound is prepared analogously as described for the title compound in Example 2 (step 3) using 157 mg (0.20 mmol) 2-{(1R,2S)-1-[2-((S)-8-Carboxy-8-cyclopentyloxycarbonylamino-octylamino)-benzenesulfonyl-aminocarbonyl]-2-vinyl-cyclopropylcarbamoyl}-3-aza-bicyclo[3.1.0]hexane-3-carboxylic acid tert-butyl ester and 0.5 mL TFA in 5 mL DCM.

HPLC (method A) t_{R}=4.43 min

TLC, Rf (CH₂Cl₂/MeOH 9:1)=0.37

MS (method D): 674 [M+]

### Example 27

### (1R,2S,22′R,23a′S)-6′,6′-Dioxido-1′,4′,19′-trioxo-2-vinylicosahydrodispiro[cyclopropane-1,3′-pyrrolo[2,1-g][1,2,5,8]thiatriazacyclohenicosine-7′,1″-cyclopropan]-22′-yl 4 fluoro-1,3-dihydro-2H-isoindole-2-carboxylate

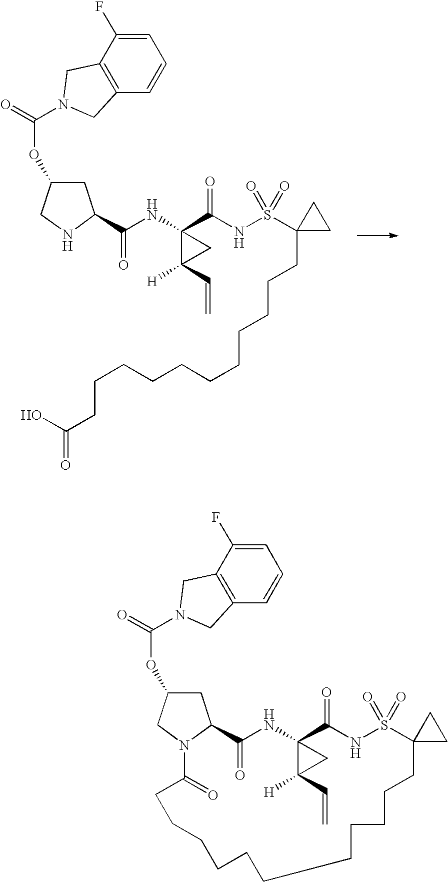

The title compound is prepared in analogy to the procedure described in Example 1 (last step) using 115 mg (0.14 mmol) 4-Fluoro-1,3-dihydro-isoindole-2-carboxylic acid (3R,5S)-5-{(1R,2S)-1-[1-(11-carboxy-undecyl)-cyclopropanesulfonylaminocarbonyl]-2-vinyl-cyclopropylcarbamoyl}-pyrrolidin-3 -yl ester (trifluoroacetate)

LC MS (method E) t_{R}=5.135 min, M+H=687.3

HPLC (method C) t_{R}=5.681 min

### Preparation of 4-Fluoro-1,3-dihydro-isoindole-2-carboxylic acid (3R,5S)-5-{(1R,2S)-1-[1-(11-carboxy-undecyl)-cyclopropanesulfonylaminocarbonyl]-2-vinyl-cyclopropylcarbamoyl}-pyrrolidin-3-yl ester (trifluoroacetate)

Step 1

### (12-Bromo-dodecyloxy)-tert-butyl-dimethyl-silane

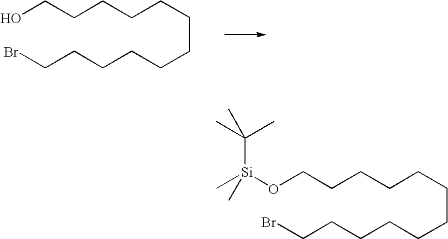

To a mixture of 3.8 g (14.3 mmol) 12-Bromo-1-dodecanol and 1.2 g (17.2 mmol) imidazole in 8 mL DMF is added 2.6 g (17.2 mmol) tert-Butyl-chloro-dimethyl-silane. The mixture is stirred at RT for 5 h, then EtOAc is added and the mixture is washed with 1N aq. HCl and water. The combined organic phases are dried over Na₂SO₄ and concentrated in vacuo to give the product which was used in the next step without further purification.

TLC, Rf (EtOAc/hexane 1:9)=0.70

Step 2

### 1-[12-(tert-Butyl-dimethyl-silanyloxy)-dodecyl]-cyclopropanesulfonylamine tert-butyl carbamate

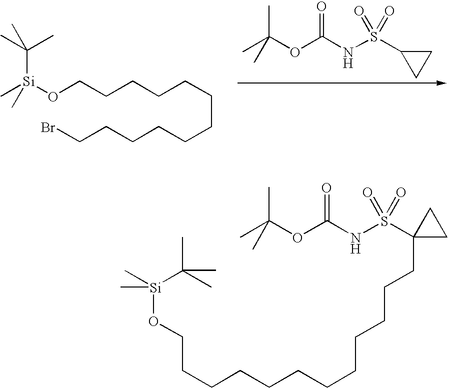

To an ice-cold solution of 4.0 mL (28.2 mmol) diisopropylamine in 45 mL THF is added 17 mL (27 mmol) n-BuLi (1.6 M in hexanes). The mixture is stirred for 1 h at 0° C. and cooled to −78° C. A mixture of 2.4 g (10.8 mmol) Cyclopropylsulfonylamine tert-butyl carbamate (prepared as described in US2007/0010455) in 5 mL THF is added and the resulting mixture is stirred for an additional hour. Then 4.5 g (11.9 mmol) (12-Bromo-dodecyloxy)-tert-butyl-dimethyl-silane is added and the mixture is allowed to warm to RT and stirred overnight. Sat. aq. NH₄Cl-solution is added and the mixture is extracted with EtOAc. The combined organic layers are dried over Na₂SO₄ and concentrated in vacuo. The residue is purified by FC on silica (eluent: hexane to EtOAc/hexane 1:1) to give the title compound.

TLC, Rf (EtOAc/hexane 1:9)=0.8

Step 3

### 1-(12-Hydroxy-dodecyl)-cyclopropanesulfonylamine tert-butyl carbamate

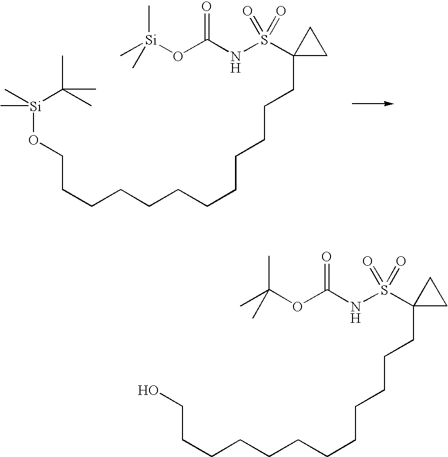

A mixture of 3.3 g (6.4 mmol) 1-[12-(tert-Butyl-dimethyl-silanyloxy)-dodecyl]-cyclopropanesulfonyl-amine tert-butyl carbamate and 13 mL TBAF (1 M in THF) in 400 mL THF is stirred for 4 h at RT. Sat. aq. NH₄Cl-solution is added and the mixture is extracted with EtOAc. The combined organic layers are dried over Na₂SO₄ and concentrated in vacuo. The residue is purified by FC on silica (eluent: hexane to EtOAc/hexane 1:1) to give the title compound.

TLC, Rf (EtOAc/hexane 1:1)=0.45

Step 4

### 1-(12-Oxo-dodecyl)-cyclopropanesulfonylamine tert-butyl carbamate

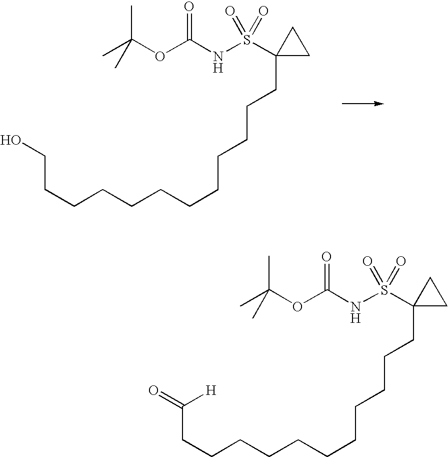

The title compound is prepared in analogy to the procedure described in Example 14 (step 3) using 1.8 g (4.4 mmol) 1-(12-Hydroxy-dodecyl)-cyclopropanesulfonylamine tert-butyl carbamate, 1.4 g (6.7 mmol) PCC in 150 mL DCM.

TLC, Rf (EtOAc/hexane 1:19)=0.7

Step 5

### 12-(1-tert-Butyl carbamoylsulfamoyl-cyclopropyl)-dodecanoic acid

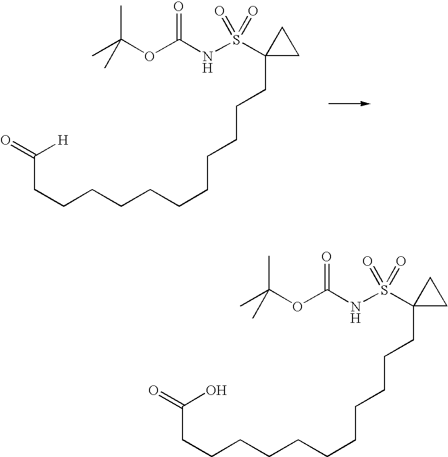

The title compound is prepared in analogy to the procedure described in Example 16 (step 1) using 1.5 g (3.7 mmol) 1-(12-Oxo-dodecyl)- cyclopropanesulfonylamine tert-butyl carbamate.

TLC, Rf (EtOAc/hexane 1:19)=0.42

Step 6

### 12-(1-Sulfamoyl-cyclopropyl)-dodecanoic acid methyl ester

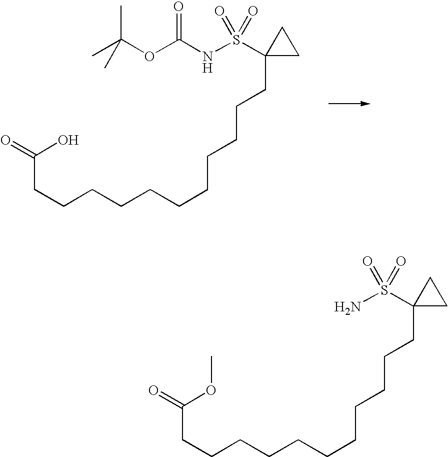

A mixture of 1.5 g (3.6 mmol) 12-(1-tert-Butyl carbamoylsulfamoyl-cyclopropyl)-dodecanoic acid in 10 mL MeOH is cooled to −15° C. and 1.7 mL (23.6 mmol) thionylchloride is added. The mixture is stirred for 1 h at RT and heated to 60° C. overnight. At RT 1 mL of thionylchloride is added and the mixture is again warmed to 60° C. for 2 h before it is concentrated and filtered over a small plug of silica gel to give the title compound.

TLC, Rf (EtOAc/hexane 1:1)=0.57

Step 7

### 12-{1-[((1R,2S)-1-tert-Butoxycarbonylamino-2-vinyl-cyclopropanecarbonyl)-sulfamoyl]-cyclopropyl}-dodecanoic acid methyl ester

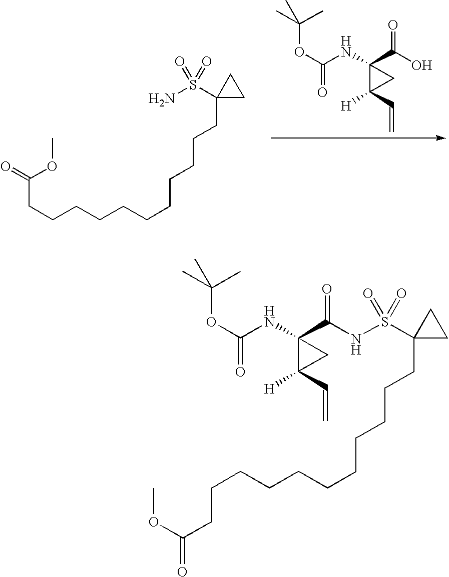

A mixture of 610 mg (2.7 mmol) (1R,2S)-1-tert-Butoxycarbonylamino-2-vinyl-cyclopropane-carboxylic acid and 687 mg (4.0 mmol) CDI in 20 mL THF is refluxed for 1 h. The reaction mixture is cooled to RT and 0.6 mL (4.0 mmol) DBU and a mixture of 806 mg (2.4 mmol) 12-(1-Sulfamoyl-cyclopropyl)-dodecanoic acid methyl ester in 5 mL THF is added. The mixture is stirred at RT overnight, concentrated in vacuo, taken up in EtOAc and washed with 0.1 M aq. HCl. The combined organic phases are dried over Na₂SO₄ and concentrated in vacuo. The residue is purified by FC on silica (Eluent: EtOAc/hexane 1:3) to give the title compound.

LC-MS (method E) t_{R}=5.132 min, M−H=543.3

HPLC (method C) t_{R}=4.472 min

Step 8

### 12-{1-[((1R,2S)-1-Amino-2-vinyl-cyclopropanecarbonyl)-sulfamoyl]-cyclopropyl}-dodecanoic acid methyl ester (hydrochloride)

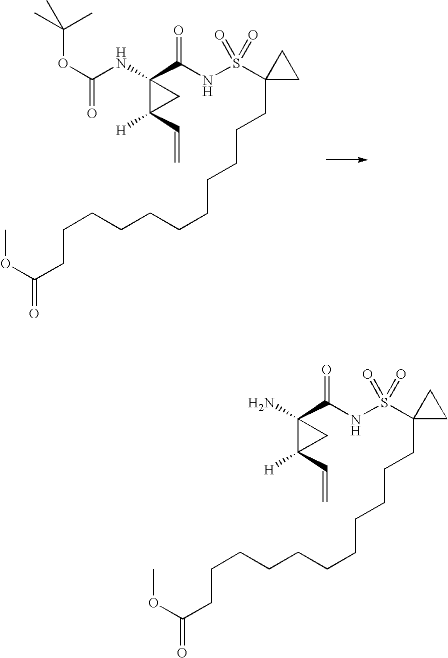

A mixture of 343 mg (0.6 mmol) 12-{1-[((1R,2S)-1-tert-Butoxycarbonylamino-2-vinyl-cyclopropane-carbonyl)-sulfamoyl]-cyclopropyl}-dodecanoic acid methyl ester and 10 mL of a 4 M solution of HCl in dioxane in 10 mL dioxane is stirred at RT overnight. The mixture is concentrated and coevaporated twice with DCM. The obtained product is used without further purification.

LC MS (method E) t_{R}=4.103 min, M−H=443.2

HPLC (method C) t_{R}=3.258 min

Step 9

### 4-Fluoro-1,3-dihydro-isoindole-2-carboxylic acid (3R,5S)-1-tert-butoxycarbonyl-5-{(1R,25)-1-[1-(11-methoxycarbonyl-undecyl)-cyclopropanesulfonylaminocarbonyl]-2-vinyl-cyclopropylcarbamoyl}-pyrrolidin-3-yl ester

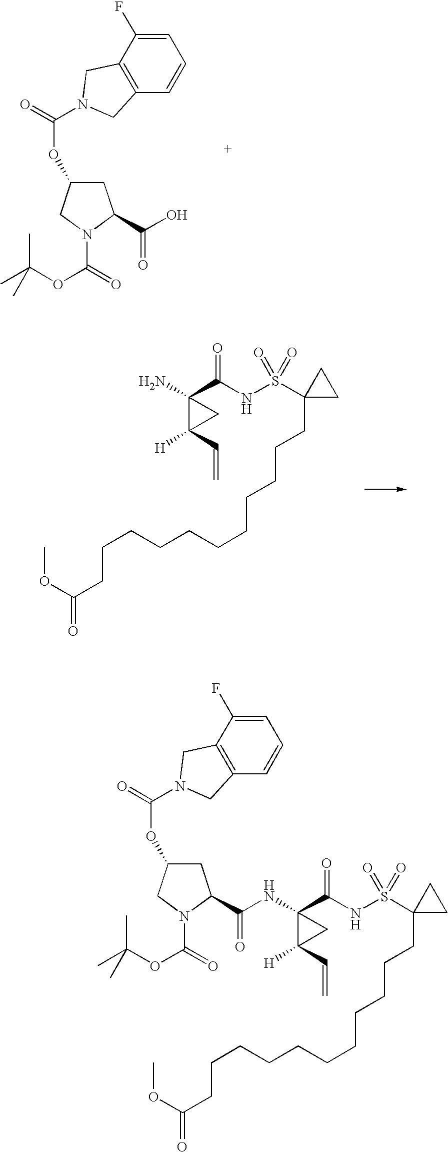

To a mixture of 181 m (0.46 mmol) (2S,4R)-4-(4-Fluoro-1,3-dihydro-isoindole-2-carbonyloxy)-pyrrolidine-1,2-dicarboxylic acid 1-tert-butyl ester in 3 mL DMF is added 0.2 mL (1.25 mmol) DIPEA and 192 mg (0.50 mmol) HBTU at RT. After 30 min 200 mg (0.42 mmol) 12-{1-[((1R,2S)-1-Amino-2-vinyl-cyclopropanecarbonyl)-sulfamoyl]-cyclopropyl}-dodecanoic acid methyl ester (hydrochloride) is added and the mixture is stirred at RT overnight. DCM is added and the mixture is washed with aq. K₂CO₃-solution. The aq. layer is extracted twice with DCM and the combined organic layers are washed with aq. 10% KHSO₄-solution and brine, dried over Na₂SO₄ and concentrated under reduced pressure. The residue is purified by FC (silica gel, eluent: EtOAc/hexane 1:3) to give the title compound.

LC MS (method E) t_{R}=4.317 min, M+H=819.4

HPLC (method C) t_{R}=4.681 min

Step 9

### 4-Fluoro-1,3-dihydro-isoindole-2-carboxylic acid (3R,5S)-1-tert-butoxycarbonyl-5-{(1R,2S)-1-[1-(11-carboxy-undecyl)-cyclopropanesulfonylaminocarbonyl]-2-vinyl-cyclopropylcarbamoyl}-pyrrolidin-3-yl ester

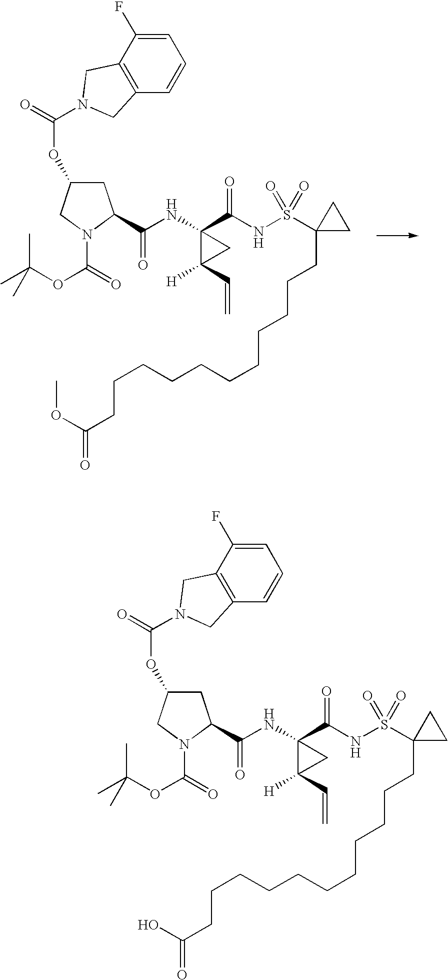

A mixture of 137 mg (0.17 mmol) 4-Fluoro-1,3-dihydro-isoindole-2-carboxylic acid (3R,5S)-1-tert-butoxycarbonyl-5-{(1R,2S)-1-[1-(11-methoxycarbonyl-undecyl)-cyclopropanesulfonylaminocarbonyl]-2-vinyl-cyclopropylcarbamoyl}-pyrrolidin-3-yl ester and 21 mg (0.50 mmol) Lithiumhydroxid-monohydrate in 2 mL THF/MeOH/water (2:1:1) is stirred at RT overnight. The mixture is concentrated under reduced pressure, the residue is acidified with 1N HCl and extracted with DCM (3×). The combined organic layers are dried over Na₂SO₄ and concentrated in vacuo to give the title compound which is used without further purification.

LC MS (method E) t_{R}=4.623 min, M+H=805.3

Step 10

### 4-Fluoro-1,3-dihydro-isoindole-2-carboxylic acid (3R,5S)-5-{(1R,2S)-1-[1-(11-carboxy-undecyl)-cyclopropanesulfonylaminocarbonyl]-2-vinyl-cyclopropylcarbamoyl}-pyrrolidin-3-yl ester (trifluoroacetate)

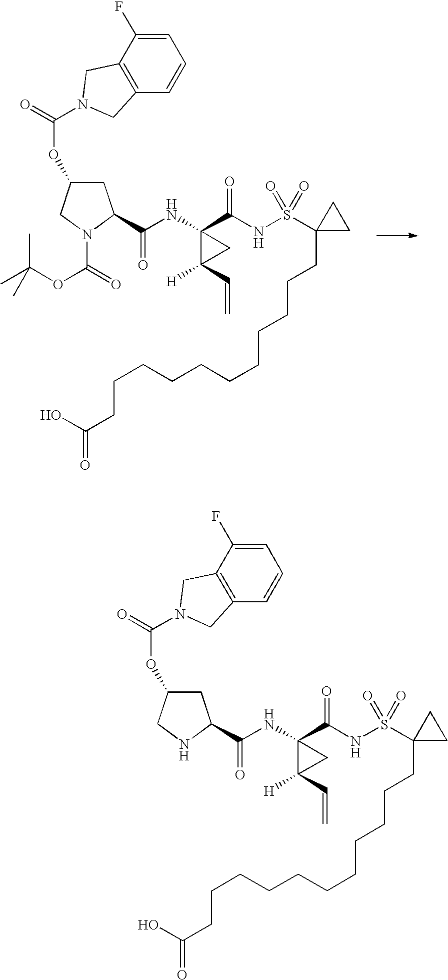

A mixture of 115 mg (0.14 mmol) 4-fluoro-1,3-dihydro-isoindole-2-carboxylic acid (3R,5S)-1-tert-butoxycarbonyl-5-{(1R,2S)-1-[1-(11-carboxy-undecyl)-cyclopropanesulfonylaminocarbonyl]-2-vinyl-cyclopropylcarbamoyl}-pyrrolidin-3-yl ester and 0.2 mL (2.93 mmol) TFA in 2 mL DCM is stirred at RT for 1.5 h before the mixture is concentrated in vacuo. The crude product is used without further purification.

LC MS (method E) t_{R}=3.316 min, M+H=705.3

### Example 28

### (1R,2S,22′R,23a′S)-7′-methyl-6′,6′-dioxido-1′,4′,19′-trioxo-2-vinylicosahydro-7′H-spiro[cyclopropane-1,3′-pyrrolo[2,1-g][1,2,5,8,21]thiatetraazacyclohenicosin]-22′-yl4-fluoro-1,3-dihydro-2H-isoindole-2-carboxylate

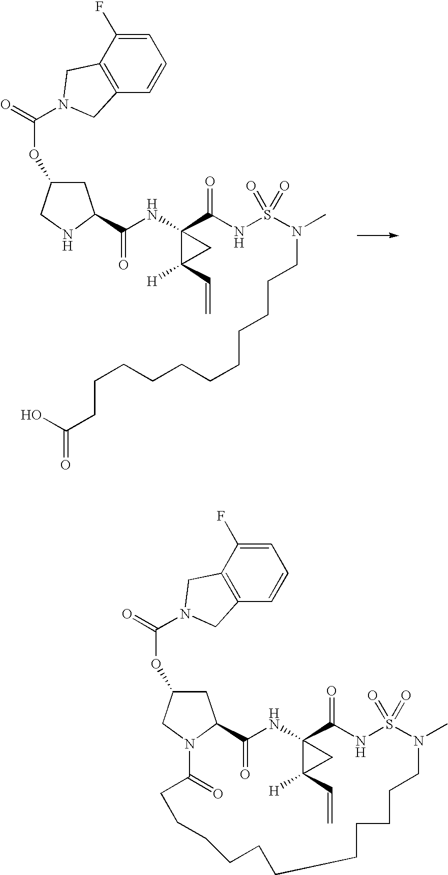

The title compound is prepared in analogy to the procedure described in Example 1 (last step) using 700 mg (0.75 mmol) of the title compound obtained in step 8 (trifluoroacetate)

LC MS (method E) t_{R}=4.613 min, M−H=674.2

HPLC (method C) t_{R}=4.275 min

Step 1

### 12-Methylamino-dodecanoic acid methyl ester

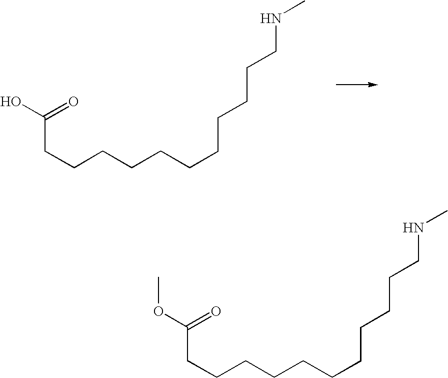

To a mixture of 5 g (21.8 mmol) 2-Methylamino-dodecanoic acid in 25 mL MeOH is added 5.5 mL (620 mmol) thionyl chloride at −15° C. The reaction mixture is refluxed overnight and concentrated under reduced pressure to yield the title compound which is used without further purification.

LC MS (method E) t_{R}=1.819 min, M+H=244.3

Step 2

### Methyl 12-[{[(tert-butoxycarbonyl)amino]sulfonyl}(methyl)amino]dodecanoate

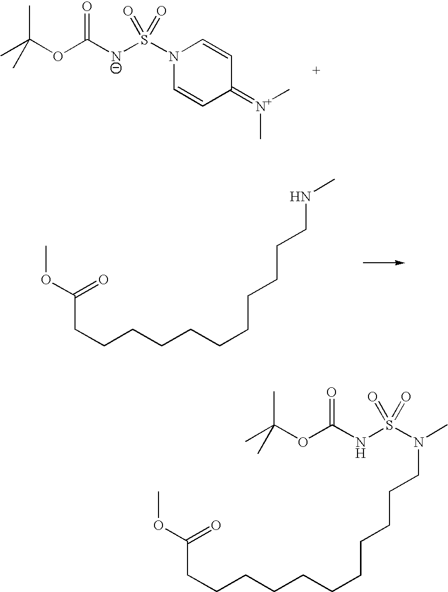

A mixture of 100 mg (0.33 mmol) N-(tert-Butoxycarbonyl)-N-[4-(dimethylazaniumylidene)-1,4-dihydropyridin-1-ylsulfonyl]azanide (prepared according to J.-Y. Winum et. al, Org. Lett. 2001, 3, 2241.), 97 mg (0.35 mmol) 12-Methylamino-dodecanoic acid methyl ester and 0.07 mL (0.40 mmol) DIPEA in 3 mL DCM is stirred at RT overnight. The reaction mixture is diluted with DCM and washed with 10% KHSO₄-solution. The aq. layer is extracted with DCM and the combined organic layers are washed with % KHSO₄-solution and brine, dried over Na₂SO₄ and concentrated under reduced pressure to give the title compound which is used without further purification.

LC MS (method E) t_{R}=4.415 min, M+H=423.1

Step 3

### Methyl 12-Raminosulfonyl)(methyl)amino]dodecanoate

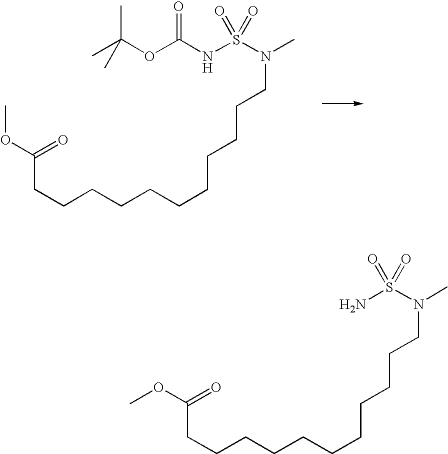

A mixture of 9 g (21 mmol) of the title compound obtained in step 2 and 25 mL (330 mmol) TFA in 100 mL DCM is stirred at RT for 1.5 h before the mixture is concentrated in vacuo. The crude product is triturated with water, filtered, dried and used without further purification.

LC MS (method E) t_{R}=4.00 min, M+H=321.1

Step 4

### Methyl 12-[{[({(1R,2S)-1-(tert-butoxycarbonyl)amino]-2-vinylcyclopropyl}carbonyl)amino]-sulfonyl}(methyl)amino]dodecanoate

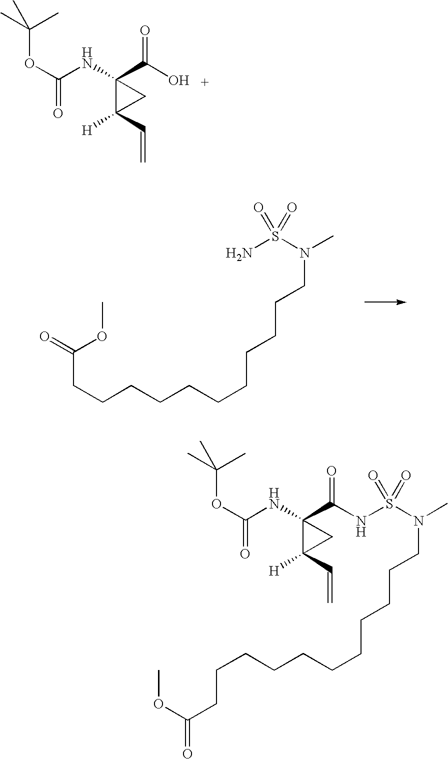

A mixture of 1.41 g (6.2 mmol) (1R,2S)-1-tert-Butoxycarbonylamino-2-vinyl-cyclopropane-carboxylic acid and 1.52 mg (9.31 mmol) CDI in 30 mL THF is refluxed for 1 h. In a second flask to a mixture of 3.0 g (9.31 mmol) of the title compound obtained in step 3 in 30 mL THF 9.3 mL (9.3 mmol) LiHMDS (1 M in THF) is added at 0° C. and the mixture is stirred for 30 min. Both mixtures are combined and stirred at RT overnight. Water is added and the mixture is extracted with DCM (3×). The combined organic layers are dried over Na₂SO₄ and concentrated in vacuo. The residue is purified by FC (silica gel, eluent: EtOAc/hexane 1:3) to give the title compound.

LC-MS (method E) t_{R}=4.728 min, M−H=530.2

Step 5

### Methyl 12-{[({[(1R,2S)-1-amino-2-vinylcyclopropyl]carbonyl}amino)sulfonyl]-(methyl)amino}-dodecanoate (hydrochloride)

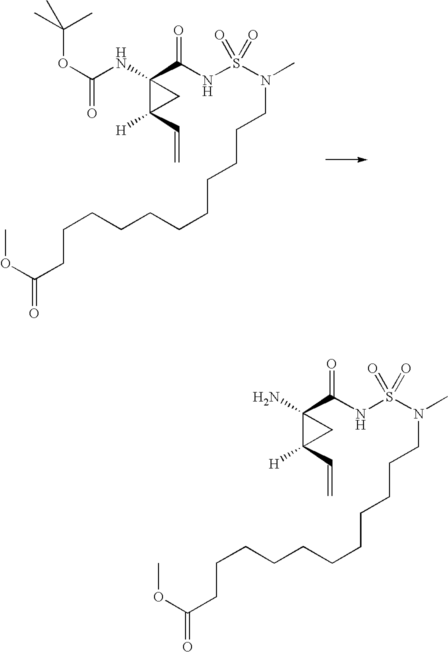

A mixture of 1.91 g (3.6 mmol) of the title compound obtained in step 4 and 18 mL of a 4 M solution of HCl in dioxane in 18 mL dioxane is stirred at RT for 6 h. The mixture is concentrated and coevaporated twice with DCM. The obtained product is used without further purification.

LC MS (method E) t_{R}=3.642 min, M+H=432.3

Step 6

### (3R,5S)-1-(tert-butoxycarbonyl)-5-{[(1R,2S)-1-({[(12-methoxy-12-oxododecyl)(methyl)amino]-sulfonyl}carbamoyl)-2-vinylcyclopropyl]carbamoyl}pyrrolidin-3-yl 4-fluoro-1,3-dihydro-2H-isoindole-2-carboxylate

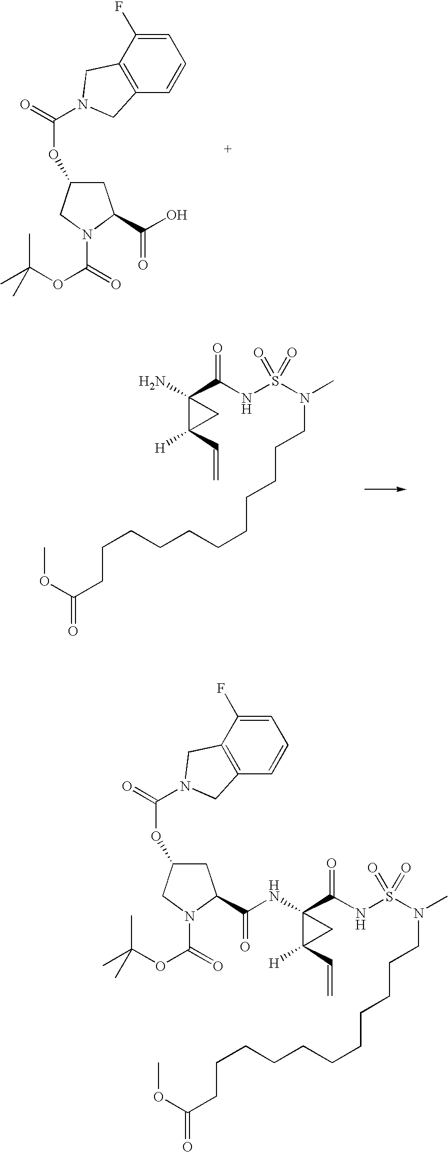

To a mixture of 447 mg (1.13 mmol) of the title compound obtained in step 5 in 10 mL DMF is added 0.5 mL (3.09 mmol) DIPEA and 474 mg (1.24 mmol) HBTU at RT. After 30 min 569 mg (1.03 mmol) 12-{1-[((1R,2S)-1-Amino-2-vinyl-cyclopropanecarbonyl)-sulfamoyl]-cyclopropyl}-dodecanoic acid methyl ester (hydrochloride) is added and the mixture is stirred at RT overnight. DCM is added and the mixture is washed with aq. K₂CO₃-solution. The aq. layer is extracted twice with DCM and the combined organic layers are washed with aq. 10% KHSO₄-solution and brine, dried over Na₂SO₄ and concentrated under reduced pressure. The residue is purified by FC (silica gel, eluent: EtOAc/hexane 1:1) to give the title compound.

LC MS (method E) t_{R}=5.007 min, M+H=806.3

### Step 7

### 12-{[({[(1R,2S)-1-{[(4R)-1-(tert-butoxycarbonyl)-4-{[(4-fluoro-1,3-dihydro-2H-isoindol-2-yl)carbonyl]oxy}-L-prolyl]amino}-2-vinylcyclopropyl]carbonyl}amino)sulfonyl]-(methyl)amino}-dodecanoic acid

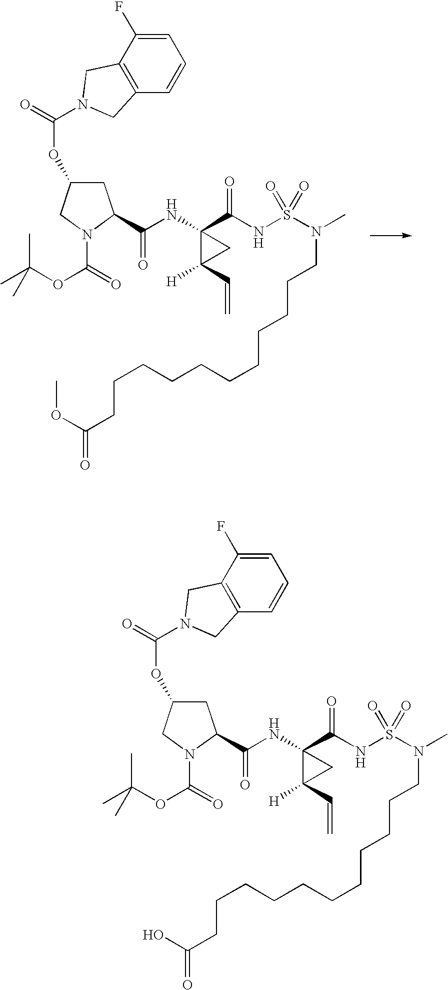

A mixture of 641 mg (0.79 mmol) of the title compound obtained in step 6 and 100 mg (2.38 mmol) Lithiumhydroxid-monohydrate in 8 mL THF/MeOH/water (2:1:1) is stirred at RT overnight. The mixture is concentrated under reduced pressure, the residue is acidified with 1N HCl and extracted with DCM (3×). The combined organic layers are dried over Na₂SO₄ and concentrated in vacuo to give the title compound which is used without further purification.

LC MS (method E) t_{R}=4.574 min, M−H=792.4

Step 8

### 12-{[({[(1R,2S)-1-{[(4R)-4-{[(4-fluoro-1,3-dihydro-2H-isoindol-2-yl)carbonyl]oxy}-L-prolyl]amino}-2-vinylcyclopropyl]carbonyl}amino)sulfonyl](methyl)amino}dodecanoic acid

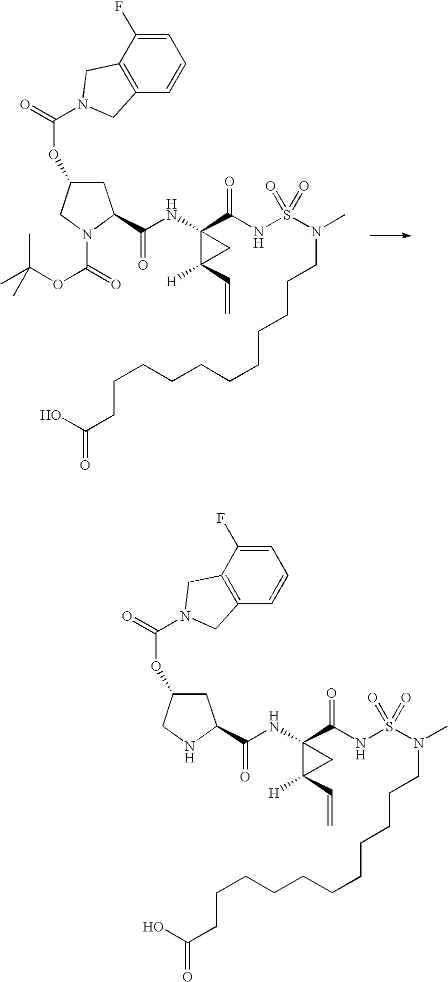

A mixture of 600 mg (0.76 mmol) of the title compound obtained in step 7 and 0.5 mL (6.5 mmol) TFA in 12 mL DCM is stirred at RT for 1.5 h, before the mixture is concentrated in vacuo. The crude product is used without further purification.

LC MS (method E) t_{R}=3.023 min, M=H=692.2

### Example 29

### Cyclopentyl [(1S,2″S,61′S,22′R,24a′S)-2,2-dimethyl-19′,19′-dioxido-5′,21′,24′-trioxo-2″-vinyl-1′,5′,6′,7′,8′,9′,10′,11′,12′,13′,14′,20′,21′,23′,24′,24a′-hexadecahydrodispiro[cyclopropane-1,2′-pyrrolo[2,1-g][1,2,5,8,18]benzothiatetraazacycloicosine-22′,1″-cyclopropan]-6′-yl]carbamate

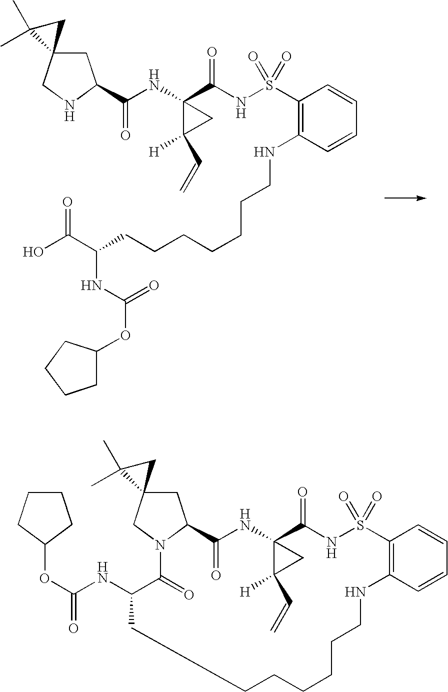

The title compound is prepared analogously as described for the title compound in Example 2 using 330 mg (0.35 mmol) ((S)-2-Cyclopentyloxycarbonylamino-9-[2-({(1R,2S)-1-[((3S,6S)-1,1-dimethyl-5-aza-spiro[2.4]heptane-6-carbonyl)-amino]-2-vinyl-cyclopropanecarbonyl}-sulfamoyl)-phenylamino]-nonanoic acid (TFA-salt), 452 mg (3.5 mmol) DIPEA and 665 mg (1.75 mmol) HATU in 75 mL DCM and 1.5 mL DMF.

HPLC (method A) t_{R}=6.21 min

TLC, Rf (CH₂Cl₂/MeOH 19:1)=0.40

MS (method D): 698 [M+]

### Preparation of (3S,6S)-1,1-Dimethyl-5-aza-spiro[2.4]heptane-5,6-dicarboxylic acid 5-tert-butyl ester

Step 1

### (3R,7aS)-6-Hydroxymethyl-3-phenyl-tetrahydro-pyrrolo[1,2-c]oxazol-5-one

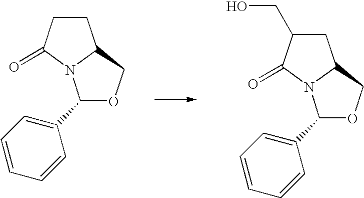

To a solution of DIPA (12.4 mL, 88.6 mmol, 1.2 equiv) in THF (400 mL) at −30° C. is added n-BuLi (50 mL, 1.60 M in hexane, 81.0 mmol, 1.10 equiv). The solution is stirred at this temperature for 30 min, then a solution of (3R,7aS)-3-Phenyl-tetrahydro-pyrrolo[1,2-c]oxazol-5-one (15.0 g, 73.8 mmol, 1.0 equiv, prepared according to J. Org. Chem. 1986, 51, 3140.) is added and the solution is stirred at −30° C. for 30 min.

A stream of CHO (22.0 g, 738 mmol, 10 equiv) and N2 gas is bubbled through this solution over 10 mins. The reaction mixture is warmed up to 0° C. over 30 mins and quenched by addition of 2.0 N HCl aq. solution until pH 3. EtOAc is added and the phases are separated. The aqueous layer is extracted 3× with EtOAc, the combined organic layer is washed with brine, dried over Na₂SO₄ and concentrated. The residue was continued to the next step with no further purification.

Step 2

### (3R,7aS)-6-Methylene-3-phenyl-tetrahydro-pyrrolo[1,2-c]oxazol-5-one

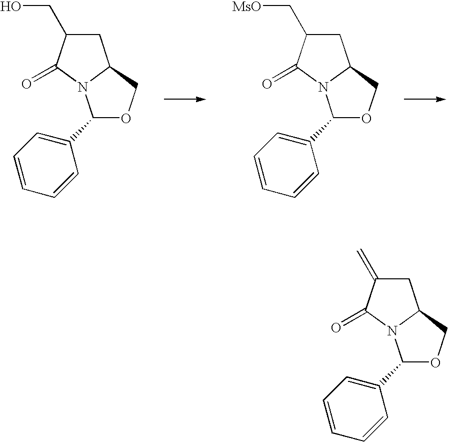

The residue from step 1 is dissolved in DCM (200 mL). To this solution at 0° C. is added TEA (30.9 mL, 222 mmol, 3.0 equiv), DMAP (902 mg, 7.4 mmol, 0.1 equiv), followed by SLOW addition of MsCl (11.5 mL, 148 mmol, 2.0 equiv), while the reaction temperature is maintained below 5° C. The solution is stirred at rt for 2 h, quenched by addition of sat. aq. NH₄Cl and followed by 1/1 mixture of EtOAc/TBME. The phases are separated and the aqueous layer is extracted with EtOAc. The organic layers are combined, washed with brine, dried with Na₂SO₄ and concentrated.

The residue is dissolved in DCM/toluene (20 mL/20 mL). At 0° C., 15 mL of DBU are added and the internal temperature is kept below 20° C. After stirring for 2 h at RT the mixture is loaded directly to a silical gel column and flushed with hexane/EtOAc (2/1 to 1/1) to give the title compound (7.4 g). The product is used immediately in the next step to avoid polymerization.

LC-MS (method E) t_{R}=0.86 min, M+H=216.1

Step 3

### 1S,3′R,7a′S)-2,2-dimethyl-3′-phenyldihydro-1′H-spiro[cyclopropane-1,6′-pyrrolo[1,2-c[1,3]oxazol]-5′-one

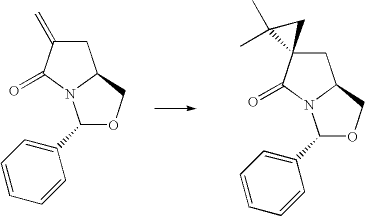

To a solution of isopropyl triphenyl phosphine iodide (10.4 g, 24.1 mmol, 1.4 equiv) in THF (70 mL) at −30° C. is added n-BuLi (1.60 M, 13.9 mL, 22.4 mmol). The solution is stirred at 0° C. for 30 min, then cooled to −30° C. A solution of (3R,7aS)-6-Methylene-3-phenyl-tetrahydro-pyrrolo[1,2-c]oxazol-5-one (3.7 g, 17.2 mmol, 1.0 equiv) is and the reaction is warmed to rt over 1 h and stirred at rt for 3 h. The reaction is quenched by addition of sat. aq. NaHCO₃ solution. After diltution with EtOAc, the mixture is filtered. The two phases are separated and the aqueous layer is extracted with EtOAc. Organic layers are combined, washed with brine, dried over Na₂SO₄ and concentrated. The residue is purified by silical gel, hexane/EtOAc 3/1 to 2/1 to give the title compound.

TLC, Rf (EtOAc/heptane 1:2)=0.53 (diastereomer 1) and 0.46 (diastereomer 2)

Step 5

### ((3S,6S)-5-Benzyl-1,1-dimethyl-5-aza-spiro[2.4]hept-6-yl)-methanol

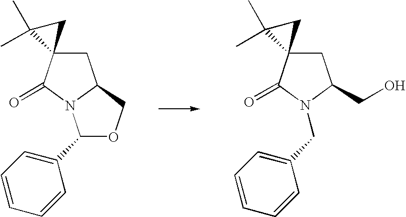

To an ice-cold solution of 9.9 g (38 mmol) (1S,3′R,7a′S)-2,2-dimethyl-3′-phenyldihydro-1′H-spiro[cyclopropane-1,6′-pyrrolo[1,2-c][1,3]oxazol]-5′-one in 250 mL abs. THF is added 4.52 g (115 mmol) LiAlH₄ under Argon. The reaction is refluxed for 3 h and quenched at 0° C. by addition of 10 mL sat. aq. Na₂SO₄. After addition of 300 mL EtOAc and stirring for 30 min the mixture is filtered and the filtrate is concentrated to give the titled compound, which is used without further purification.

HPLC (method A) t_{R}=2.64 min

TLC, Rf (CH₂Cl₂/MeOH 9:1)=0.48

MS (method D): 246 [M+H]

Step 6

((3S,6S)-1,1-Dimethyl-5-aza-spiro[2.4]hept-6-yl)-methanol

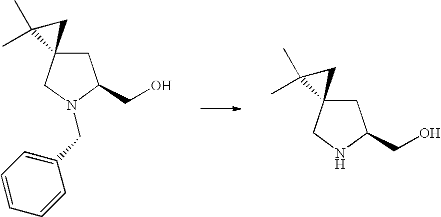

A suspension of 9.5 g (38 mmol) ((3S,6S)-5-Benzyl-1,1-dimethyl-5-aza-spiro[2.4]hept-6-yl)-methanol and 10% Pd on charcoal (2 g) in 100 mL EtOAc/AcOH (1:1) is stirred for 2.5 h under H₂ atmosphere. The reaction is filtered, washed with DCM and concentrated. After addition of 2N aq. NaOH, the aq. phase is extracted with DCM. The combined organic phases are washed with brine, dried with Na₂SO₄, filtered and the solvent is removed in vacuo. The residue is purified by FC (silica gel, eluent: DCM/MeOH 9:1→4:1) to give the title compound.

TLC, Rf (CH₂Cl₂/MeOH 4:1)=0.29

MS (method D): 156 [M+H]

Step 7

### (3S,6S)-6-Hydroxymethyl-1,1-dimethyl-5-aza-spiro[2.4]heptane-5-carboxylic acid tert-butyl ester

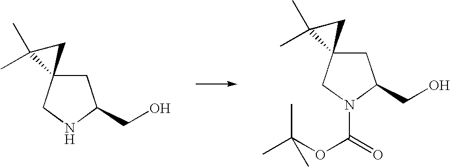

To an ice-cold solution of 1.4 g (9.0 mmol) ((3S,6S)-1,1-Dimethyl-5-aza-spiro[2.4]hept-6-yl)-methanol in 30 mL DCM is added 2.5 mL (18 mmol) NEt3 and 2.8 g (12.6 mmol) (BOC)₂0 and the mixture is stirred overnight at RT. The reaction is quenched by addition of aq. sat. bicarbonate and extracted with DCM. The combined organic phases are washed with brine, dried with Na₂SO₄, filtered and the solvent is removed in vacuo. The residue is purified by FC (silica gel, eluent: DCM/MeOH 19:1) to give the title compound.

TLC, Rf (CH₂Cl₂/MeOH 19:1)=0.58

MS (method D): 200 [M-55]

Step 8

### (3S,6S)-1,1-Dimethyl-5-aza-spiro[2.4]heptane-5,6-dicarboxylic acid 5-tert-butyl ester

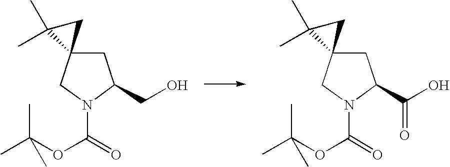

To a solution of 1.7 g (6.7 mmol) (3S,6S)-6-Hydroxymethyl-1,1-dimethyl-5-aza-spiro[2.4]heptane-5-carboxylic acid tert-butyl ester in 30 mL DCM is added 235 mg (0.67 mmol) TPAP, 1.18 g (10 mmol) NMO followed by 300 mg molecular sieves 4A. The reaction is stirred for 2 h at RT, filtered through a pad of Celite, washed with DCM and the solvent is removed in vacuo. The residue is dissolved in 30 mL tert-butanol and 2.4 g (33.3 mmol) 2-Methyl-2-buten is added, followed by 3.1 g (20 mmol) NaH₂PO₄ (in 20 mL water) and 1.81 g (20 mmol) NaCl O₂ (in 20 mL water). After 2 h at RT, 0.5 N aq. HCl is added and extracted with EtOAc. The solvent is removed in vacuo, the residue is dissolved in DCM and extracted 3× with aq. NaHCO₃. The organic phase is discarded, while the bicarbonate phase is acidified with 4 N HCl to pH 1-2 and then extracted with DCM. The combined organic phase is dried with Na₂SO₄, filtered and the solvent is removed in vacuo to give the title compound, which is used without further purification.

TLC, Rf (CH₂Cl₂/MeOH 19:1)=0.16

MS (method D): 214 [M-55]

### Preparation of ((S)-2-Cyclopentyloxycarbonylamino-9-[2-({(1R,2S)-1-[((3S,6S)-1,1-dimethyl-5-aza-spiro[2.4]heptane-6-carbonyl)-amino]-2-vinyl-cyclopropanecarbonyl}-sulfamoyl)-phenylamino]-nonanoic acid

Step 1

### (35,6S)-6-{(1R,2S)-1-[2-(S)-8-Cyclopentyloxycarbonylamino-8-methoxycarbonyl-octylamino)-benzenesulfonylaminocarbonyl]-2-vinyl-cyclopropylcarbamoyl}-1,1-dimethyl-5-aza-spiro[2.4]heptane-5-carboxylic acid tert-butyl ester

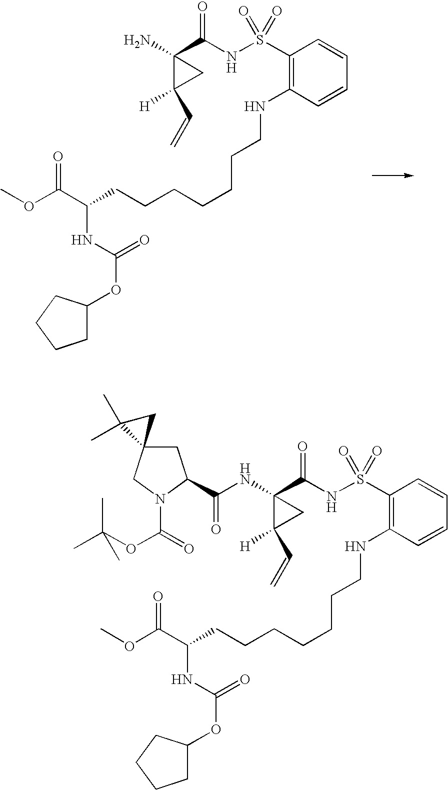

The title compound is prepared analogously as described for the title compound in Example 2 (step 1) using 403 mg (0.50 mmol) (S)-9-{2-[((1R,2S)-1-Amino-2-vinyl-cyclopropanecarbonyl)-sulfamoyl]-phenylamino}-2-cyclopentyloxycarbonylamino-nonanoic acid methyl ester (TFA-salt), 162 mg (0.60 mmol) (3S,6S)-1,1-Dimethyl-5-aza-spiro[2.4]heptane-5,6-dicarboxylic acid 5-tert-butyl ester, 285 mg (0.75 mmol) HATU and 388 mg (3.0 mmol) DIPEA in 15 mL DCM.

HPLC (method A) t_{R}=6.30 min

TLC, Rf (CH₂Cl₂/MeOH 9:1)=0.40

MS (method D): 830 [M+]

Step 2

### (3S,6S)-6-{((1R,2S)-1-[2-(S)-8-Carboxy-8-cyclopentyloxycarbonylamino-octylamino)-benzenesulfonylaminocarbonyl]-2-vinyl-cyclopropylcarbamoyl}-1,1-dimethyl-5-aza-spiro[2.41heptane-5-carboxylic acid tert-butyl ester

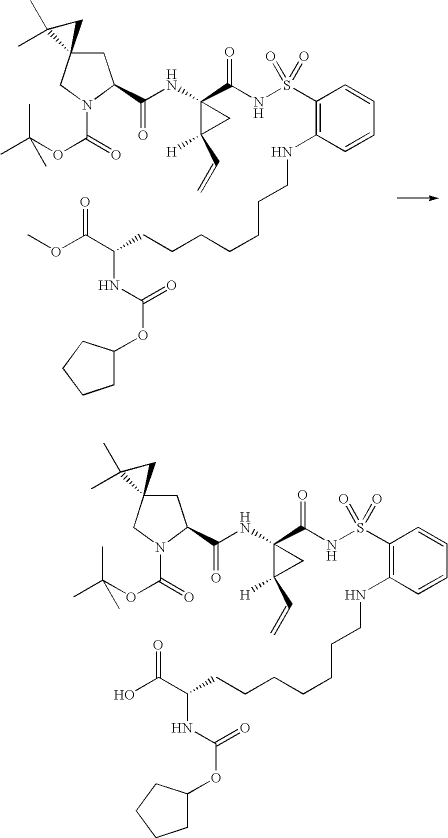

The title compound is prepared analogously as described for the title compound in Example 2 (step 2) using 330 mg (0.35 mmol) (3S,6S)-6-{(1R,2S)-1-[2-((S)-8-Cyclopentyloxycarbonylamino-8-methoxycarbonyl-octylamino)-benzenesulfonylaminocarbonyl]-2-vinyl-cyclopropylcarbamoyl}-1,1-dimethyl-5-aza-spiro[2.4]heptane-5-carboxylic acid tert-butyl ester (TFA-salt) and 84 mg (3.5 mmol) LiOH in 20 mL THF/MeOH/H₂O (2:1:1).

HPLC (method A) t_{R}=5.85 min

TLC, Rf (CH₂Cl₂/MeOH 9:1)=0.50

MS (method D): 816 [M+]

Step 3

### (S)-2-Cyclopentyloxycarbonylamino-9-[2-({(1R,2S)-1-[((3S,6S)-1,1-dimethyl-5-aza-spiro[2.4]heptane-6-carbonyl)-amino]-2-vinyl-cyclopropanecarbonyl}-sulfamoyl)-phenylamino]-nonanoic acid

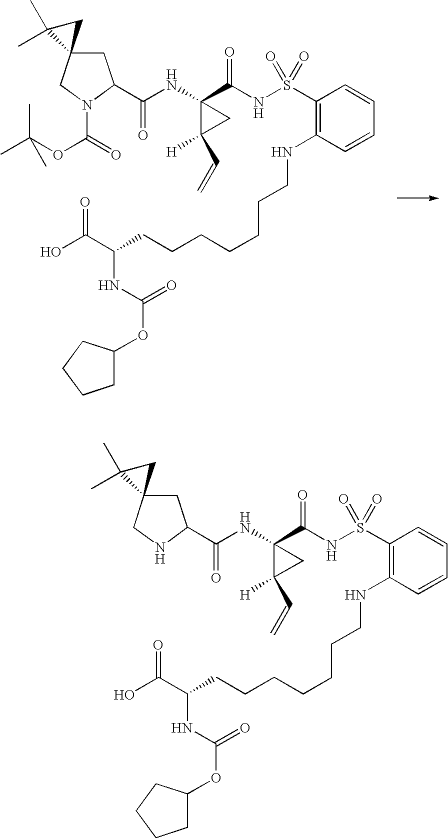

The title compound is prepared analogously as described for the title compound in Example 2 (step 3) using 258 mg (0.35 mmol) (3S,6S)-6-{(1R,2S)-1-[2-((S)-8-Carboxy-8-cyclopentyloxycarbonylamino-octylamino)-benzenesulfonylaminocarbonyl]-2-vinyl-cyclopropylcarbamoyl}-1,1-dimethyl-5-aza-spiro[2.4]heptane-5-carboxylic acid tert-butyl ester and 1.0 mL TFA in 10 mL DCM.

HPLC (method A) t_{R}=4.87 min

TLC, Rf (CH₂Cl₂/MeOH 85:15)=0.73

MS (method D): 716 [M+]

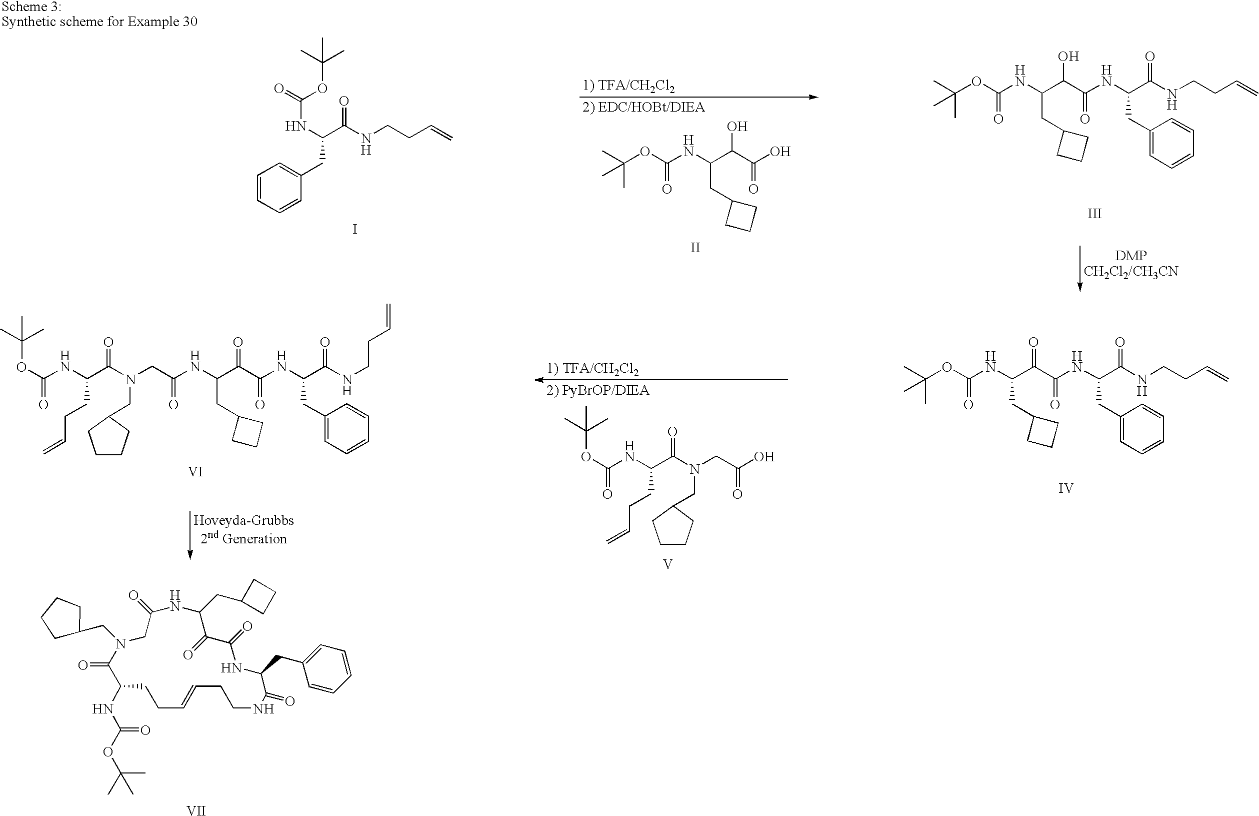

### Example 30

### ((E)-(3S,13S)-3-Benzyl-7-cyclobutylmethyl-11-cyclopentylmethyl-2,5,6,9,12-pentaoxo-1,4,8,11tetraaza-cyclononadec-16-en-13-yl)-carbamic acid tert-butyl ester

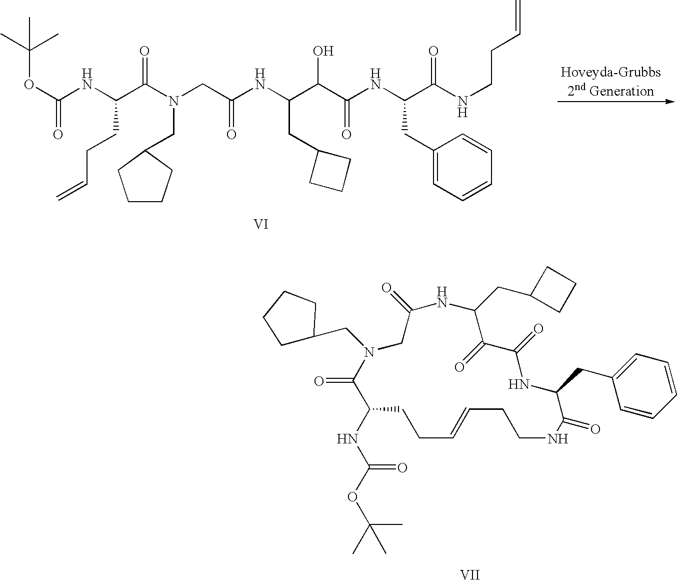

A solution of VI (85 mg, 0.12 mmol,) with Hoveyda-Grubbs 2nd generation catalyst (3 mg, ˜3 mol %) in Toluene (10 mL) degassed with N₂ is heated to 80° C. for 2.5 hours. After 2.5 hours the reaction is cooled to room temp and the catalyst is scavenged by adding the reaction to thiourea bound resin (4 equiv.). The reaction is stirred for 1 hour after which time the solution is filtered and the solvent removed. The crude product is run though a plug of silica gel with EtOAc and is purified by prep HPLC to yield VII.

LC-MS (method E): M+H=694.9

### Preparation of [(S)-1-({[2-((S)-1-But-3-enylcarbamoyl-2-phenyl-ethylcarbamoyl)-1-cyclobutylmethyl-2-oxo-ethylcarbamoyl]-methyl}-cyclopentylmethyl-carbamoyl)-pent-4-enyl]-carbamic acid tert-butyl ester VI Step 1

### [2-((S)-1-But-3-enylcarbamoyl]-2-phenyl-ethylcarbamoyl)-1-cyclobutylmethyl-2-hydroxy-ethyl]-carbamic acid tert-butyl ester

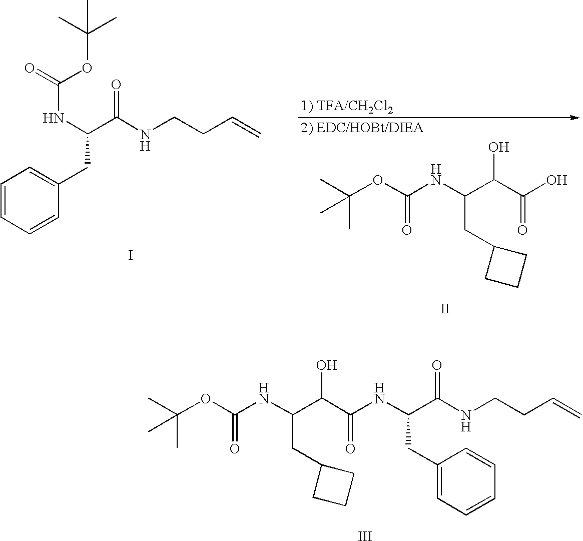

To a solution of I (500 mg, 1.57 mmol, 1.0 equiv) in CH₂Cl₂ (2.0 mL) at 0° C. is added (2.0 mL) and the solution is stirred at room temp for 1 hour. After 1 hour the solvent is removed under reduced pressure to yield a crude oil. A solution of II (640 mg, 2.30 mmol, 1.5 equiv), EDC (0.45 g, 2.30 mmol, 1.5 equiv), DIEA (2.0 mL, 11.5 mmol, 7.5 equiv) in CH₂Cl₂ (5.0 mL) is added at 0° C.. The solution is brought to room temperature and stirred for 18 hours. The reaction mixture is diluted with EtOAc and washed with 0.5 N HCl. The phases are separated and the aqueous layer is extracted with EtOAc. The organic layers are combined and washed with brine, dried over Na₂SO₄ and concentrated. The residue is purified by silica gel column chromatography (heptane/EtOAc, 1:3) to give product III.

LC-MS (method E): M+H=474.3

Step 2:

### [(S)-2-(S)-1-But-3-enylcarbamoyl-2-phenyl-ethylcarbamoyl)-1-cyclobutylmethyl-2-oxo-ethyl]-carbamic acid tert-butyl ester

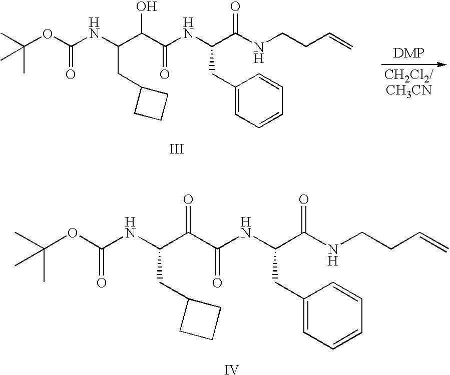

To a solution of III (150 mgs, 0.32 mmol, 1.0 equiv) a in CH₃CN (10.0 mL) at ° C. is added DMP (0.39 mgs, 2.5 equiv.) and the solution is stirred at room temp for 1 hour. After 1 hour 3 mL 1 N sodium thiosulfate is added to the reaction mixture and the solution extracted with EtOAc. The phases are separated and the aqueous layer is extracted with EtOAc. The organic layers are combined and washed with brine, dried over Na₂SO₄ and concentrated. The residue is purified by silica gel column chromatography (heptane/EtOAc, 1:1) to give product IV.

LC-MS (method E): M+H=472.3

Step 3:

### [(S)-1-({[2-((S)-1-But-3-enylcarbamoyl-2-phenyl-ethylcarbamoyl)-1-cyclobutylmethyl-2-oxo-ethylcarbamoyl]-methyl}-cyclopentylmethyl-carbamoyl)-pent-4-enyl]-carbamic acid tert-butyl ester

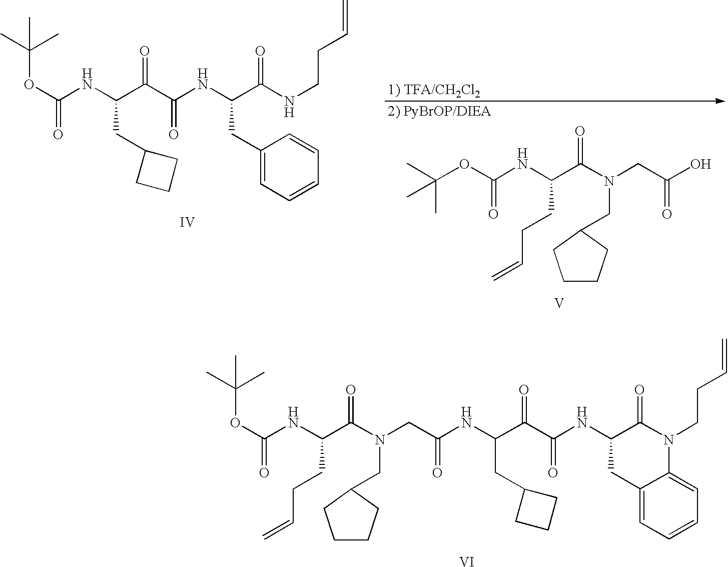

To a solution of IV (102 mg, 0.22mmol, 1.0 equiv) a in CH₂Cl₂ (2.0 mL) at 0° C. is added TFA (2.0 mL) and the solution is stirred at room temp for 1 hour. After 1 hour the solvent is removed under reduced pressure to yield a crude oil to which is added a solution of V (85 mg, 0.22 mmol, 1.0 equiv), PyBrOP (108 mgs, 0.22 mmol, 1.0 equiv), DIEA (0.2 mL, 1.15 mmol, 5 equiv) in CH₂Cl₂ (5.0 mL) at 0° C.. The solution is brought to room temperature and stirred for 18 hours. The reaction mixture is diluted with EtOAc and washed with 0.5 N HCl. The phases are separated and the aqueous layer is extracted with EtOAc. The organic layers are combined and washed with brine, dried over Na₂SO₄ and concentrated. The residue is purified by silica gel column chromatography (heptane/EtOAc, 1/3) to give product VI.

LC-MS (method E): M+H=722.9.

### Example 31

### Cyclopentyl [(1R,2S,2′R,6′S,24a′S)-2′-hydroxy-19′,19′-dioxido-5′, 21′,24′-trioxo-2-vinyl-1′,2′,3′,5′,6′,7′,8′,9′,10′,11′,12′,13′,14′,20′,21′,23′,24′,24a′-octadecahydrospiro[cyclopropane-1,22′-pyrrolo[2,1-g][1,2,5,8,18]benzothiatetraazacycloicosin]-6′-yl]carbamate

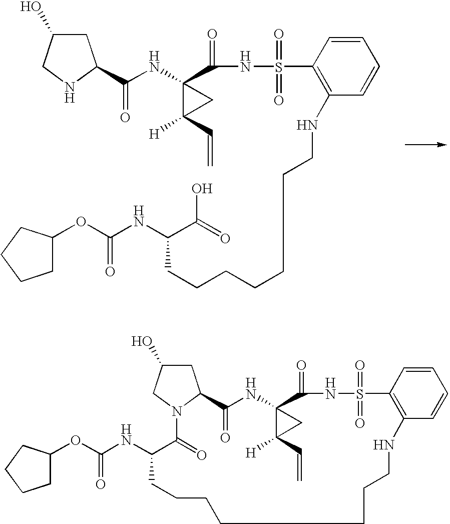

The title compound can be prepared as described above for the final step in the synthesis of example 1

LC MS (method E) t_{R}=4.209 min, M+H=660.3

HPLC (method C) t_{R}=3.993 min

Step 1

### tert-butyl (2S,4R)-4-{[tert-butyl(dimethyl)silyl]oxy}-2-{[(1R,2S)-1-{[(2-{[(8S)-8-{[(cyclopentyloxy)carbonyl]amino}-9-methoxy-9-oxononyl]amino}phenyl)sulfonyl]carbamoyl}-2-vinylcyclopropyl]carbamoyl}pyrrolidine-1-carboxylate

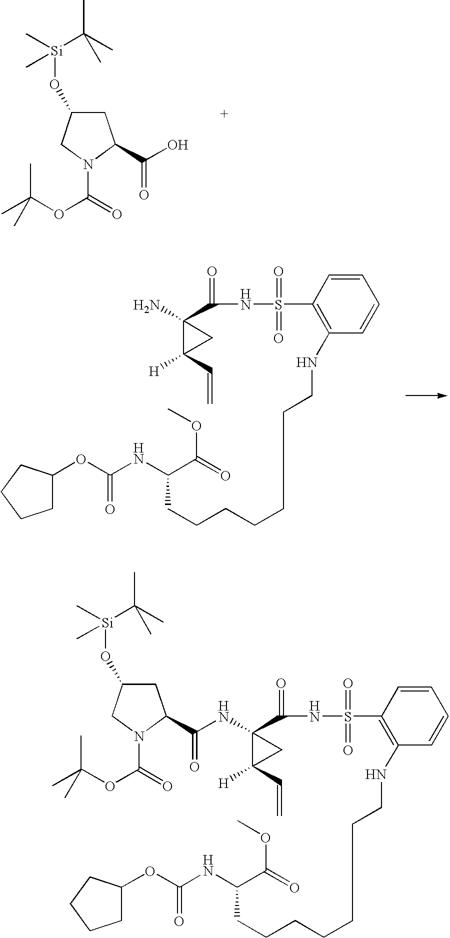

The title compound can be prepared as described above for the synthesis of (3R,5S)-1-tert-butoxycarbonyl-5-{(1R,2S)-1-[2-(8-methoxycarbonyl-octanoylamino)-benzenesulfonylaminocarbonyl]-2-vinyl-cyclopropylcarbamoyl}-pyrrolidin-3-yl ester (example 3) using (4R)-1-(tert-butoxycarbonyl)-4-{[tert-butyl(dimethyl)silyl]oxy}-L-proline (for preparation see T. Sato et al. J. Chem. Soc. Perkin Trans. 1 2001, 20, 2623).

LC MS (method E) t_{R}=5.401 min, M+H=907.2

Step 2

### (2S)-9-({2-[({[(1R,2S)-1-{[(4R)-1-(tert-butoxycarbonyl)-4-{[tert-butyl(dimethyl)silyl]oxy}-L-prolyl]amino}-2-vinylcyclopropyl]carbonyl}amino)sulfonyl]phenyl}amino)-2-{[(cyclopentyloxy)carbonyl]amino)nonanoic acid

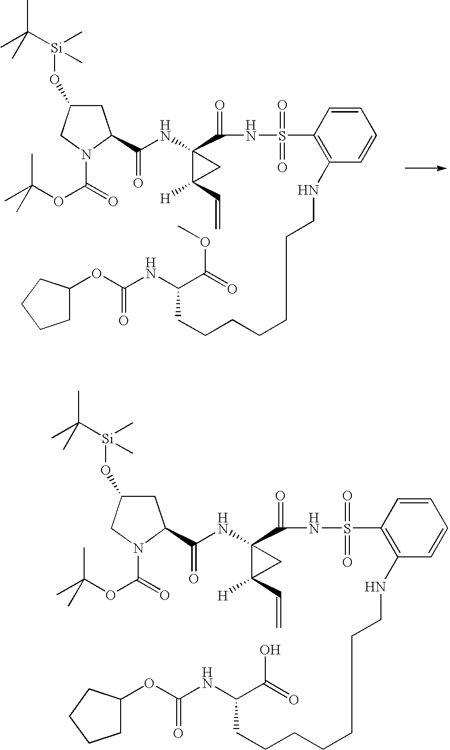

The title compound can be prepared analogously as described for the title compound in example 21, step 11.

LC MS (method E) t_{R}=5.097 min

Step 3

### (2S)-2-{[(cyclopentyloxy)carbonyl]amino}-9-({2-[({[(1R,2S)-1-{[(4R)-4-hydroxy-L-prolyl]amino}-2-vinylcyclopropyl]carbonyl}amino)sulfonyl]phenyl}amino)nonanoic acid

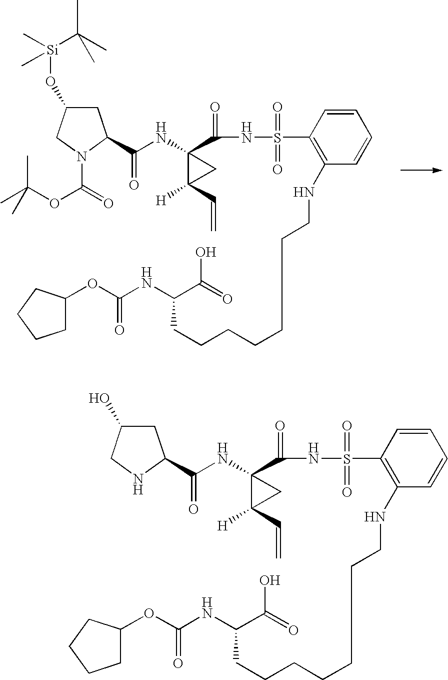

The title compound can be prepared as described above for the synthesis of (2S)-2-{[(cyclopentyloxy)carbonyl]amino) -9-({2-[(([(1R,2S)-1-{[(4R)-4-{[(2-nitrophenyl)-sulfonyl]amino}-L-prolyl]amino}-2-vinylcyclopropyl]carbonyl}amino)-sulfonyl]phenyl}-amino)nonanoic acid (hydrochloride salt).

LC MS (method E) t_{R}=3.009 min, M+H=678.3

### Example 32

### Cyclopentyl {(1R,2S,2′R,6′S,24a′S)-19′,19′-dioxido-5′,21′,24′-trioxo-2′-[(quinolin-6-ylcarbonyl)-amino]-2-vinyl-1′,2′,3′,5′,6′,7′,8′,9′,10′,11′,12′,13′,14′,20′,21′,23′,24′,24a′-octadecahydrospiro-[cyclopropane-1,22′-pyrrolo[2,1-g][1,2,5,8,18 benzothiatetraazacycloicosin]-6′-yl}carbamate

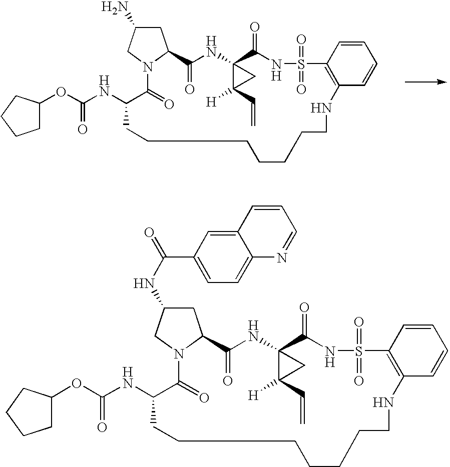

To a mixture of 50 mg (0.07 mmol) Cyclopentyl [(1R,2S,2′R,6′S,24a′S)-2′-amino-19′,19′-dioxido-5′,21′,24′-trioxo-2-vinyl-1′,2′,3′,5′,6′,7′, 8′,9′,10′,11′,12′,13′,14′,20′, 21′,23′,24′,24a′-octadecahydrospiro[cyclopropane-1,22′-pyrrolo[2,1-g][1,2,5,8,18]benzothiatetraazacycloicosin]-6′-yl]carbamate, 0.037 mL (0.21 mmol) DIPEA and 40 mg (0.11 mmol) HATU in 0.7 mL DCM/DMF (50:1) are added at 0° C. 16 mg (0.09 mmol) 6-Quinoline carboxlylic acid. The mixture is stirred for 72 h, concentrated in vacuo and purified by prep. HPLC (method C).

LC MS (method E) t_{R}=4.254 min, M+H=814.3

### Preparation of Cyclopentyl [(1R,2S,2′R,6′S,24a′S)-2′-amino-19′,19′-dioxido-5′,21′,24′-trioxo-2-vinyl-1′,2′,3′,5′,6′,7′,8′,9′,10′,11′,12′,13′,14′,20′,21′,23′,24a′-octadecahydrospiro[cyclopropane-1,22′-pyrrolo[2,1-g][1,2,5,8,18]benzothiatetraazacycloicosin]-6′-yl]carbamate

Step 1

### 1-tert-Butyl 2-methyl (2S,4R)-4-{[(2-nitrophenyl)sulfonyl]amino}pyrrolidine-1,2-dicarboxylate

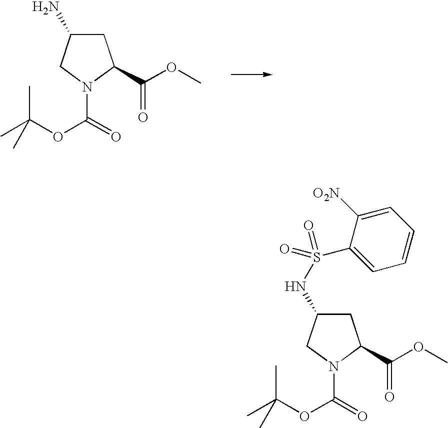

To a mixture of 3 g (10.6 mmol) N-Boc-trans-4-amino-L-proline methyl ester hydrochloride, 14.8 mL (106 mmol) triethylamine in 260 mL DCM is added 3.6 g (15.9 mmol) 2-nitro-benzolsulfonylchloride at 0° C. The mixture is stirred at rt overnight and extracted with brine. The organic layer is dried over Na₂SO₄, concentrated in vacuo and purified by FC on silica (eluent: DCM to DCMMeOH 95:5).

LC MS (method E) t_{R}=3.284 min, M+H=430.03

HPLC (method C) t_{R}=3.306 min

Step 2

### (4R)-1-(tert-butoxycarbonyl)-4-{[(2-nitrophenyl)sulfonyl]amino}-L-proline

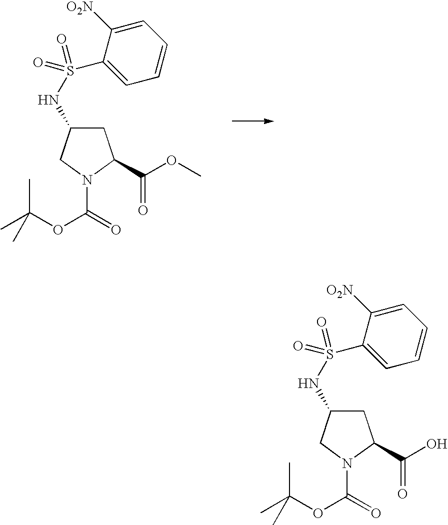

A mixture of 4.2 g (9.8 mmol) 1-tert-butyl 2-methyl (2S,4R)-4-{[(2-nitrophenyl)sulfonyl]amino}-pyrrolidine-1,2-dicarboxylate and 1.2 g (29 mmol) LiOH in 100 mL THF/water/MeOH (2:1:1) is stirred at rt for 4 h. The mixture is concentrated in vacuo and the residue is diluted with DCM and 1N aq. HCl solution. The formed precipitate is filtered and dried.

LC MS (method E) t_{R}=2.943 min, M−H=414.1

Step 3

### (2S)-2-{[cyclopentyloxy)carbonyl]amino}-9-({2-[({[(1R,2S)-1-{[(4R)-4-{[(2-nitrophenyl)-sulfonyl]amino}-L-prolyl]amino}-2-vinylcyclopropyl]carbonyl}amino-) sulfonyl]phenyl}amino)-nonanoic acid

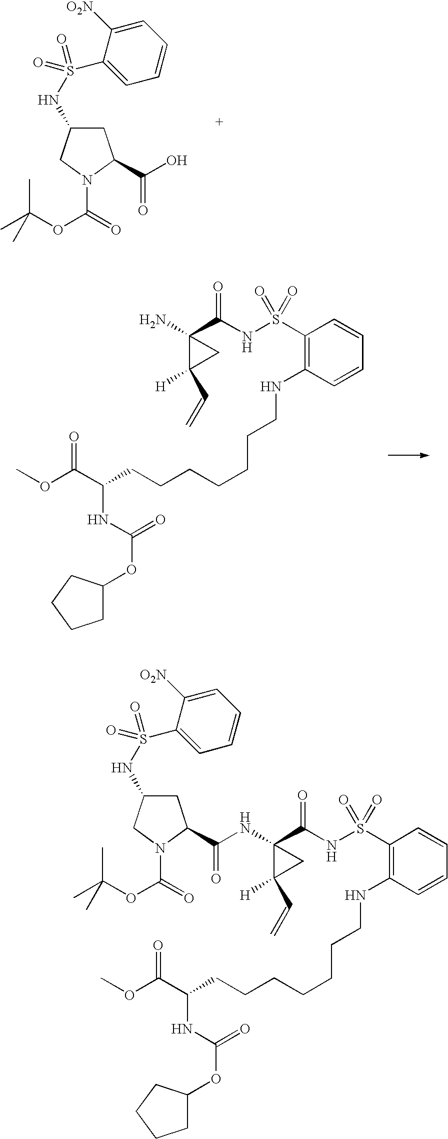

The title compound is prepared analogously as described for the title compound in example 21, step 10 using 3.3 g (5.4 mmol) methyl (2S)-9-({2-[({[(1R,2S)-1-amino-2-vinylcyclopropyl]carbonyl}amino)-sulfonyl]phenyl}amino)-2-{[(cyclopentyloxy)carbonyl]amino}nonanoate hydrochloride, 2.8 g (6.8 mmol) (4R)-1-(tert-butoxycarbonyl)-4-{[(2-nitrophenyl)sulfonyl]amino}-L-proline, 2.6 g (6.9 mmol) HTBU and 3.0 mL (17 mmol) DIPEA in 50 mL DCM/IDMF (50:1)

LC MS (method E) t_{R}=4.644 min, M+H=977.2

HPLC (method C) t_{R}=4.346 min

Step 4

### (2S)-9-({2-[({[(1R,2S)-1-{[(4R)-1-(tert-Butoxycarbonyl)-4-{[(2-nitrophenyl)sulfonyl]amino}-L-prolyl]amino}-2-vinylcyclopropyl]carbonyl}amino)sulfonyl]phenyl}amino)-2-{[(cyclopentyloxy)-carbonyl]amino}nonanoic acid

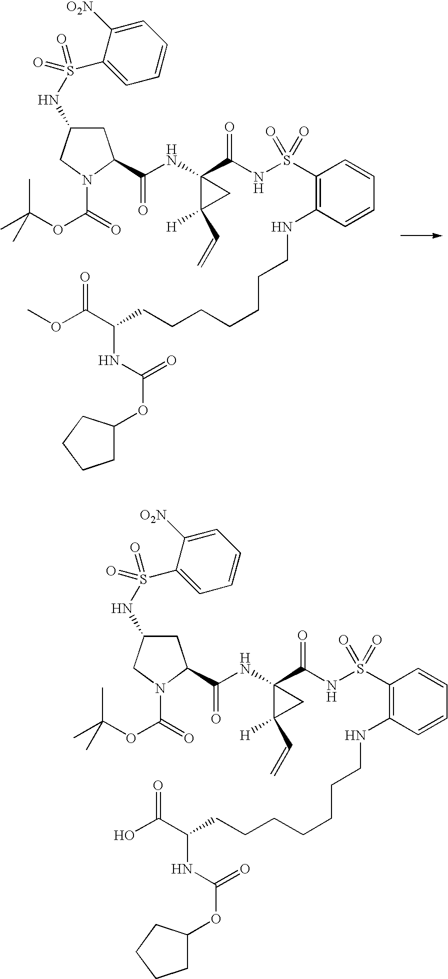

The title compound is prepared analogously as described for the title compound in example 21, step 11 using 3.0 g (3.1 mmol) ((2S)-2-{[(cyclopentyloxy)carbonyl]amino}-9-({2-[({[(1R,2S)-1-{[(4R)-4-{[(2-nitrophenyl)-sulfonyl]amino}-L-prolyl]amino}-2-vinylcyclopropyl]carbonyl}amino-)sulfonyl]phenyl}-amino)-nonanoic acid and 387 mg (9.22 mmol) LiOH in 30 mL THF/water/MeOH 2:1.:1.

LC MS (method E) t_{R}=4.240 min, M+H=963.3

HPLC (method C) t_{R}=4.083 min

Step 5

### (2S)-2-{[cyclopentyloxy)carbonyl]amino}-9-({2-[({[(1R,2S)-1-{[(4R)-4-{[(2-nitrophenyl)-sulfonyl]amino}-L-prolyl]amino}-2-vinylcyclopropyl]carbonyl}amino)-sulfonyl]phenyl}-amino)nonanoic acid (hydrochloride salt)

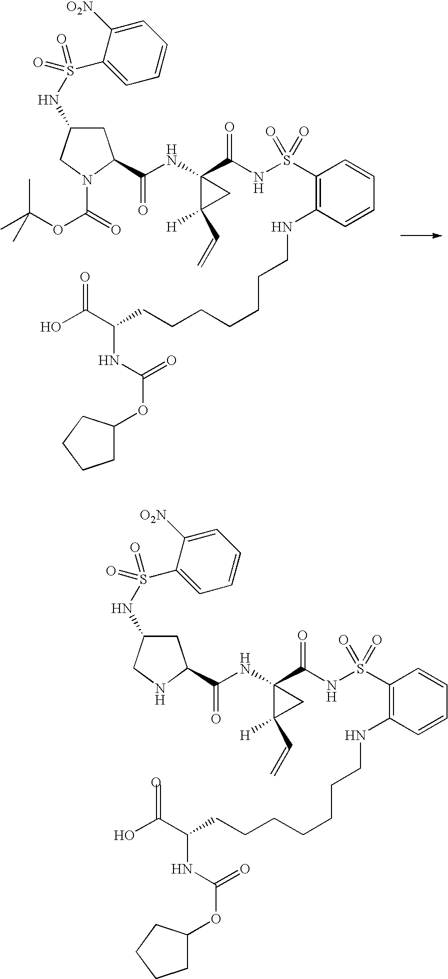

A mixture of 1.9 g (2.0 mmol) (2S)-9-({2-[({[(1R,2S)-1-{[(4R)-1-(tert-Butoxycarbonyl)-4-{[(2-nitrophenyl)sulfonyl]amino}-L-prolyl]amino}-2-vinylcyclopropyl]carbonyl)amino)sulfonyl]phenyl}amino)-2-{[(cyclopentyloxy)-carbonyl]amino}nonanoic acid, 20 mL 4 M HCl in dioxane and 20 mL dioxane is stirred at rt for 3 h. The mixture is concentrated in vacuo and the crude product is used without further purification.

LC MS (method E) t_{R}=3.346 min, M+H=863.2

HPLC (method C) t_{R}=3.484 min

Step 6

### Cyclopentyl [(1R,2S,2′R,6′S,24a′S)-2′-{[(2-nitrophenyl)sulfonyl]amino}-19′,19′-dioxido-5′,21′,24′-trioxo-2-vinyl-1′,2′,3′,5′,6′,7′,8′,9′,10′,11′,12′,13′,14′,20′, 21′,23′,24′,24a′-octadecahydrospiro-[cyclopropane-1,22′-pyrrolo[2,1-g][1,2,5,8,18]benzothiatetraazacycloicosin]-6′-yl]carbamate

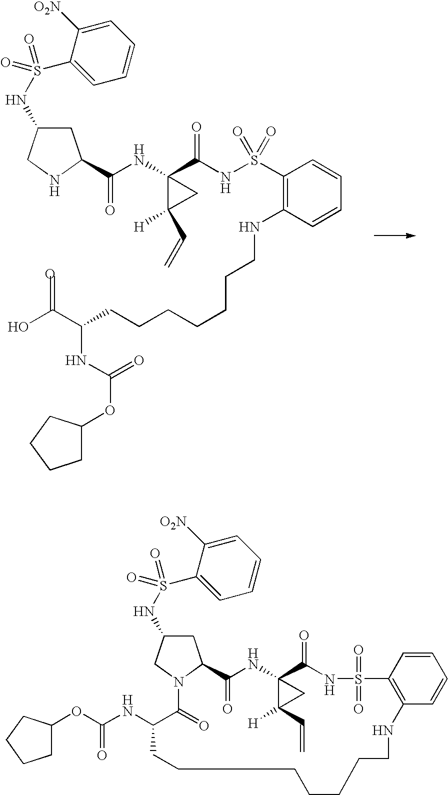

The title compound can be prepared analogously as described for the title compound of example 21 using 2.1 g (2.1 mmol) (2S)-2-{[(cyclopentyloxy)carbonyl]amino}-9-({2-[({[(1R,2S)-1-{[(4R)-4-{[(2-nitrophenyl)-sulfonyl]amino}-L-prolyl]amino}-2-vinylcyclopropyl]carbonyl}amino)-sulfonyl]phenyl}-amino)nonanoic acid (hydrochloride salt), 4.1 g (10.8 mmol) HATU and 3.8 mL (21.5 mmol) DIPEA in 300 mL DCM/DMF (50:1).

LC MS (method E) t_{R}=4.470 min, M−H=842.2

HPLC (method C) t_{R}=4.371 min

Step 7

### Cyclopentyl [(1R,2S,2′R,6′S,24a′S)-2′-amino-19′,19′-dioxido-5′,21′, 24′-trioxo-2-vinyl-1′,2′,3′,5′,6′,7′,8′,9′,10′,11′,12′,13′,14′,20′,21′,23′,24′,24a′- octadecahydrospiro[cyclopropane-1,22′-pyrrolo[2,1-g][1,2,5,8,18]benzothiatetraazacycloicosinl-6′-yl]carbamate

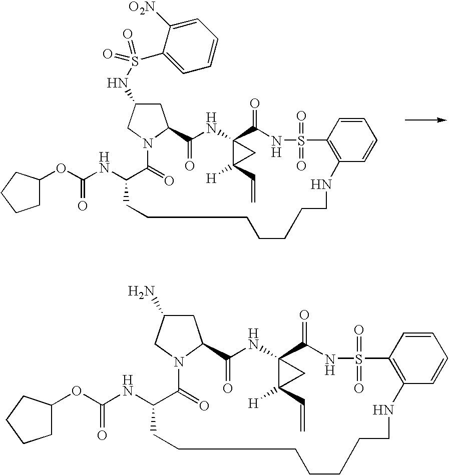

A mixture of 670 mg (0.7 mmol) Cyclopentyl [(1R,2S,2′R,6′S,24a′S)-2′-{[(2-nitrophenyl)sulfonyl]amino }-19′,19′-dioxido-5′,21′,24′-trioxo-2-vinyl-1′,2′,3′,5′,6′,7′,8′,9′,10′,11′,12′,13′,14′,20′, 21′,23′,24′,24a′-octadecahydrospiro-[cyclopropane-1,22′-pyrrolo[2,1-g][1,2,5,8,18]benzothiatetraazacycloicosin]-6′-yl]carbamate, 0.2 mL (2.2 mmol) thiophenol and 404 mg (2.9 mmol) K₂CO₃ in 30 mL acetonitrile is stirred at rt overnight. The mixture is diluted with water and ethyl acetate. The organic layer is washed with brine, dried over Na₂SO₄ and concentrated in vacuo. The residue is dissolved in hot DCM, ethyl ether is added and the precipitate is filtered and dried.

LC MS (method E) t_{R}=3.150 min, M1H=659.3

### Example 33

### (1R,2S,16′S,20′R,21a′S)-16′[(tert-butoxycarbonyl)amino]-7′-methyl-6′,6′-dioxido-1′,4′,17′-trioxo-2-vinyloctadecahydro-7′H-spiro[cyclopropane-1,3′-pyrrolo[2,1-g][1,2,5,8,19]thiatetraazacyclononadecin]-20′-yl 5-(dimethylamino)-1,3-dihydro-2H-isoindole-2-carboxylate

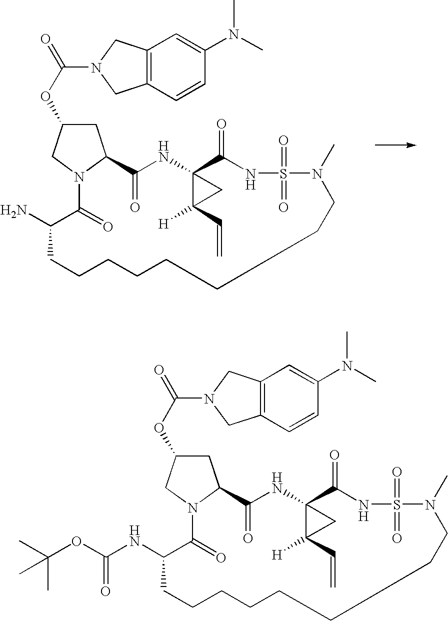

A mixture of 150 mg (0.15 mmol) (1R,2S,16′S,20′R,21a′S)-16′-amino-7′-6′6′-dioxido-1′,4′,17′-trioxo-2-vinyloctadecahydro-7′H-spiro[cyclopropane-1,3′-pyrrolo[2,1-g][1,2,5,8,19]thiatetranzacyclononadecin]-20′-yl 5-(dimethylamino)-1,3-dihydro-2H-isoindole-2-carboxylate, 37 mg (0.17 mmol) Boc2O and 0.03 mL (0.20 mmol) triethylamine in 4 mL DCM is stirred at rt overnight. The mixture is concentrated and purified by prep.

HPLC.

LC MS (method E) t_{R}=3.645 min, M+H=788.2

Step 1

### Ethyl (2S)-2-{[(2-nitrophenyl)sulfonyl]amino}dec-9-enoate

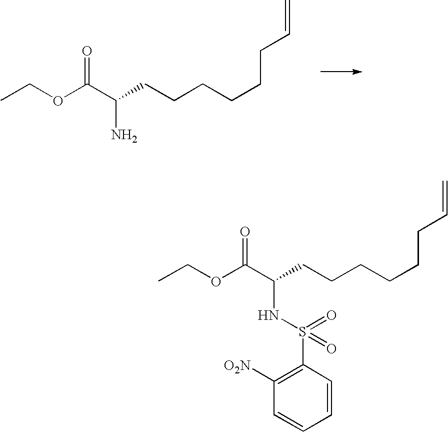

To a mixture of 10 g (47 mmol) ethyl (2S)-2-aminodec-9-enoate (prepared as described above for (S)-2-Amino-non-8-enoic acid ethyl ester) and 67 mL (469 mmol) triethylamine in 800 mL DCM is added 16 g (70 mmol) o-nitro-benzenesulfonylchloride at 0° C. The mixture is stirred at rt overnight and partitioned between EtOAc and water. The aq. layer is extracted with EtOAc and the combined organic layers are dried over Na₂SO₄ and concentrated in vacuo. The crude product is purified by FC (silica gel).

LC MS (method E) t_{R}=4.361 min, M+H=399.1

HPLC (method C) t_{R}=4.335 min

Step 2

### Ethyl (2S)-10-hydroxy-2-{[(2-nitrophenyl)sulfonyl]amino}decanoate

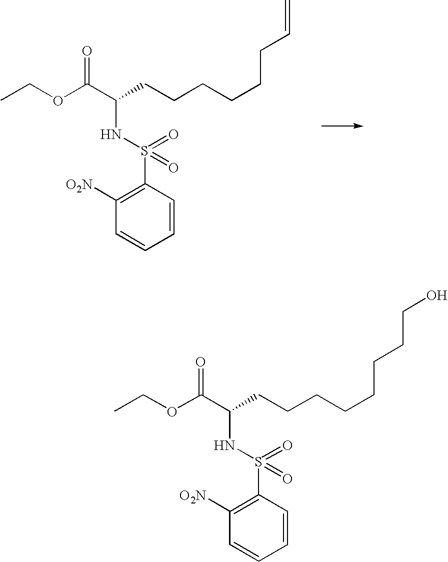

This compound can be prepared as described above for the synthesis of (S)-2-Cyclopentyloxycarbonylamino-9-hydroxy-nonanoic acid methyl ester

LC MS (method E) t_{R}=3.550 min, M+H=417.1

HPLC (method C) t_{R}=3.635 min

Step 3

### Ethyl (2S)-10-(methylamino)-2-{[(2-nitrophenyl)sulfonyl]amino}decanoate

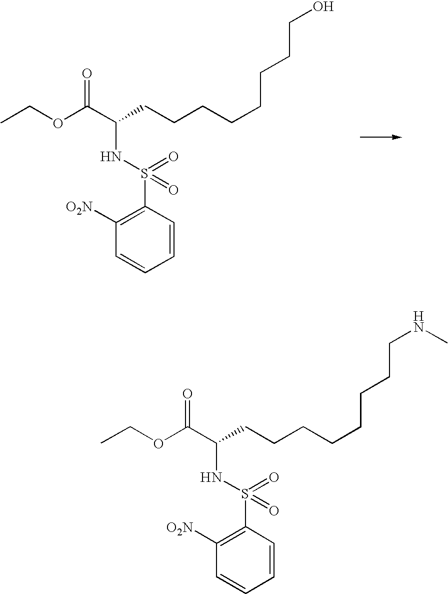

To a mixture of 13 g (31 mmol) Ethyl (25)-10-hydroxy-2-{[(2-nitrophenyl)sulfonyl]amino}decanoate in 300 mL DCM is added 2.9 mL (37 mmol) methanesulfonylchloride and 8.6 mL (61 mmol) triethylamine at 0° C. After 1 h water is added and the mixture is extracted with DCM. The combined organic layers are dried over Na₂SO₄ and concentrated. The crude is taken up in 150 mL DMSO and 42 mL methylamine (8 M in EtOH) and the mixture is stirred at rt overnight. The mixture is partitioned between water and ether and the aq. phase is extracted with ether. The combined organic layers are dried over Na₂SO₄ and concentrated in vacuo to give the title compound which is used without further purification in the next step.

LC MS (method E) t_{R}=0.930 min, M+H=430.1

Step 4

### Ethyl (2S)-10-[{[(tert-butoxycarbonyl)amino]sulfonyl}(methyl)amino]-2-{[(2-nitrophenyl)sulfonyl]amino}decanoate

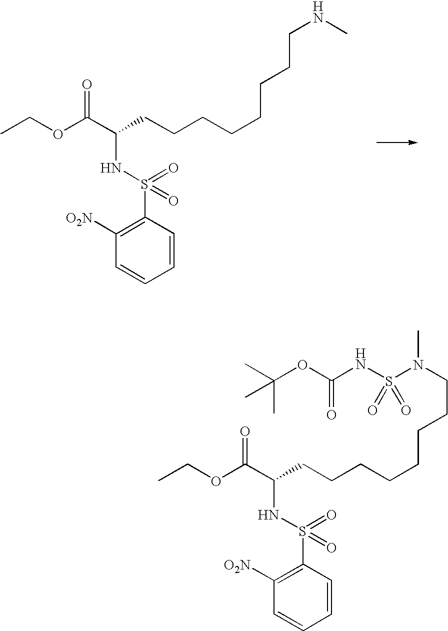

This compound can be prepared using the method described by J. Y. Winum et al. Org. Lett. 2001, 3, 2241.

LC MS (method E) t_{R}=4.121 min, M+H=609.3

HPLC (method C) t_{R}=4.580 min

Step 5

### Ethyl (2S)-10-[(aminosulfonyl)(methyl)amino]-2-{[(2-nitrophenyl)sulfonyl]amino}decanoate

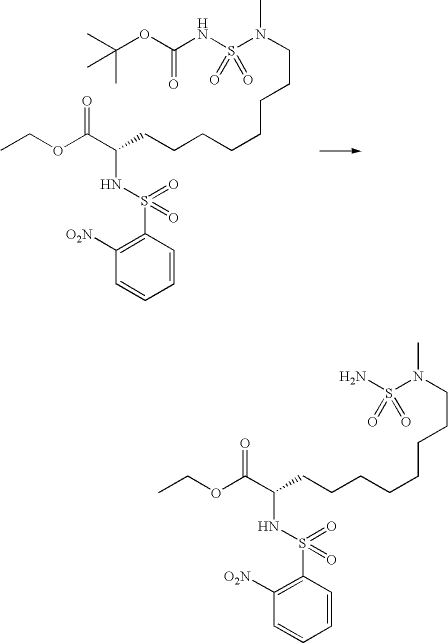

A mixture of 11 g (16 mmol) ethyl (2S)-10-[{[(tert-butoxycarbonyl)amino]sulfonyl}(methyl)amino]-2-{[(2-nitrophenyl)sulfonyl]amino}decanoate and 200 mL HCl in dioxane (4 M) is stirred overnight at rt. The mixture is concentrated and the crude is purified by FC (silica gel, eluent: hexanes to hexanes/EtOAc 1:1).

LC MS (method E) t_{R}=3.559 min, M+H=509.0

HPLC (method C) t_{R}=3.900 min

Step 6

### Ethyl (2S)-10-[{[({(1R,2S)-1-[(tert-butoxycarbonyl)amino]-2-vinylcyclopropyl}carbonyl)amino]sulfonyl}(methyl)amino]-2-{[(2-nitrophenyl)sulfonyl]amino}decanoate

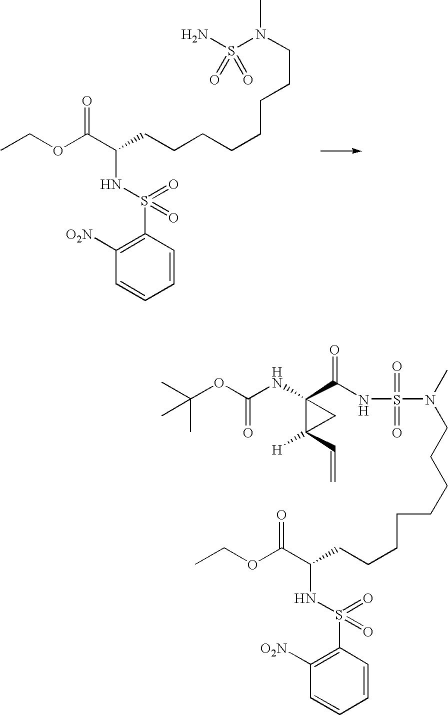

The title compound can be prepared as described above for the synthesis of [(1R,2S)-1-(2-Amino-benzenesulfonylaminocarbonyl)-2-vinyl-cyclopropyl]-carbamic acid tert-butyl ester (example 1, step 1)

LC MS (method E) t_{R}=4.270 min, M−H=718.2

HPLC (method C) t_{R}=4.289 min

Step 7

### ethyl (2S)-10-{[({[(1R,2S)-1-amino-2-vinylcyclopropyl]carbonyl}amino)sulfonyl](methyl)amino}-2-{[(2-nitrophenyl)sulfonyl]amino}decanoate

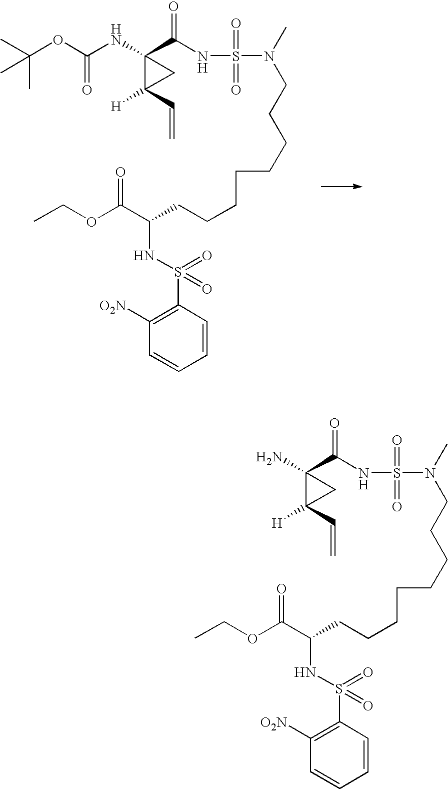

The title compound can be prepared as described above for the synthesis of 8-{2-[((1R,2S)-1-Amino-2-vinyl-cyclopropanecarbonyl)-sulfamoyl]-phenylcarbamoyl}-octanoic acid methyl ester (example 1, step 3)

LC MS (method E) t_{R}=3.368 min, M+H=618.1

HPLC (method C) t_{R}=3.279 min

Step 8

### (3R,5S)-1-(tert-butoxycarbonyl)-5-({(1R,2S)-1-[({[(9S)-10-ethoxy-9-{[(2-nitrophenyl)sulfonyl]amino}-10-oxodecyl](methyl)amino}sulfonyl)carbamoyl]-2-vinylcyclopropyl}carbamoyppyrrolidin-3-yl 5-(dimethylamino)-1,3-dihydro-2H-isoindole-2-carboxylate

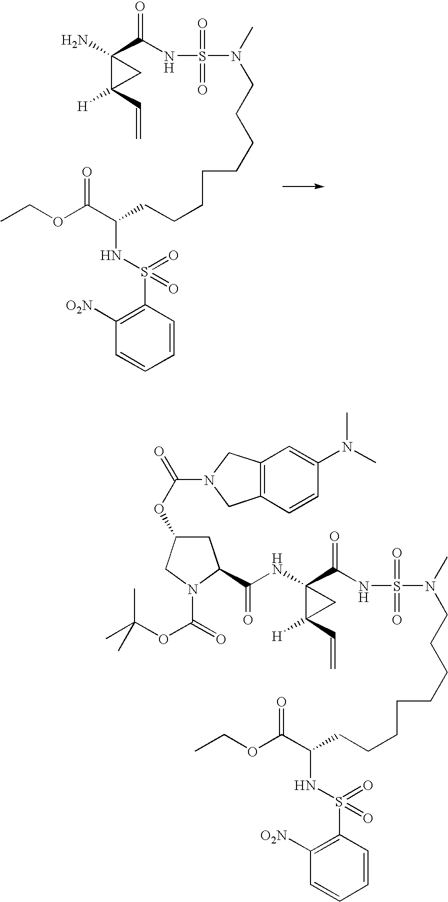

The title compound can be prepared as described above for the synthesis of 4-Fluoro-1,3-dihydro-isoindole-2-carboxylic acid (3R,5S)-1-tert-butoxycarbonyl-5-{(1R,2S)-1-[2-8-methoxycarbonyl-octanoylamino)-benzenesulfonylaminocarbonyl]-2-vinyl-cyclopropylcarbamoyl}-pyrrolidin-3-yl ester (example 3, step 4) using (4R)-1-(tert-butoxycarbonyl)-4-({[5-(dimethylamino)-1,3-dihydro-2H-isoindol-2-yl]carbonyl}oxy)-L-proline which can be prepared as described in example 3, steps 1 and 2.

LC MS (method E) t_{R}=4.439 min, M+H=1020.4

Step 9

### (2S)-10-{[({[(1R,2S)-1-{[(4R)-4-({[5-(dimethylamino)-1,3-dihydro-2H-isoindol-2-yl]carbonyl}oxy)-L-prolyl]amino}-2-vinylcyclopropyl]carbonyl)amino)sulfonyl]methyl)amino}-2-{[(2-nitrophenyl)sulfonyl]amino}decanoic acid

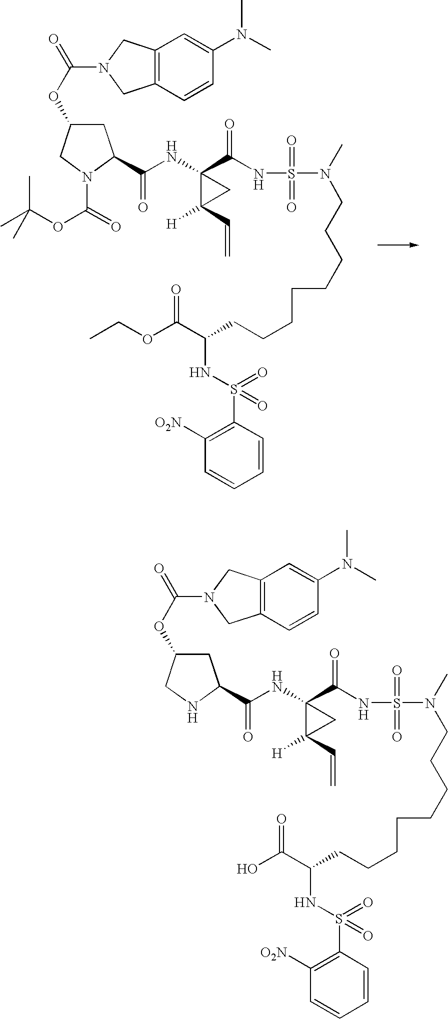

The title compound can be prepared as described above for the synthesis of (2S)-2-{[(cyclopentyloxy)carbonyl]amino}-9-({2-[({[(1R,2S)-1-{[(4R)-4-{[(2-nitrophenyl)-sulfonyl]amino}-L-prolyl]amino}-2-vinylcyclopropyl]carbonyl}amino)-sulfonyl]phenyl}-amino)nonanoic acid (hydrochloride salt) (step 4 and 5)

LC MS (method E) t_{R}=2.934 min, M+H=892.3

Step 10

### (1R,2S,16′S,20′R,21a′S)-7′-methyl-16′-{[(2-nitrophenyl)sulfonyl]amino}-6′6′-dioxido-1′,4′,17′-trioxo-2-vinyloctadecahydro-7′H-spiro[cyclopropane-1,3′-pyrrolo[2,1-g][1,2,5,8,19]thiatetraazacyclononadecin]-20′-yl]-(dimethylamino)-1,3-dihydro-2H-isoindole-2-carboxylate

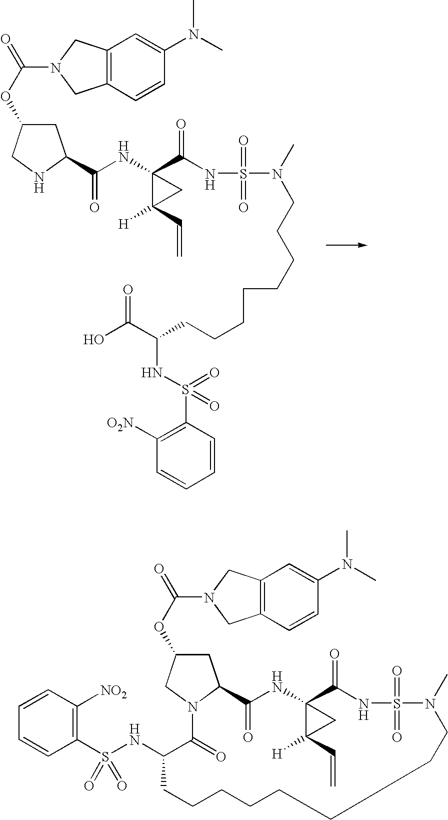

The title compound can be prepared as described above for the final step in the synthesis of example 1

LC MS (method E) t_{R}=3.927 min, M+H=874.2

Step 11

### (1R,2S,16′S,20′R,21a′S)-16′-amino-7′-methyl-6′,6′-dioxido-1′, 4′,17′-trioxo-2-vinyloctadecahydro-7′H-spiro[cyclopropane-1,3′-pyrrolo[2,1-g][1,2,5,8,19]thiatetraazacyclononadecin]-20′-yl 5-(dimethylamino)-1,3-dihydro-2H-isoindole-2-carboxylate

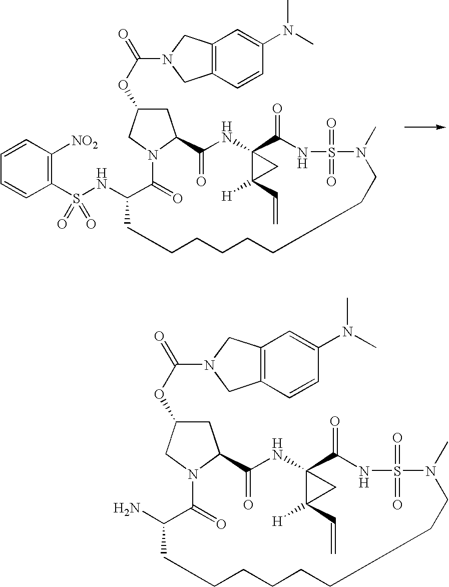

A mixture of 760 mg (0.9 mmol) (1R,2S,16′S,20′R,21a′S)-7′-methyl-16′-{[(2-nitrophenyl)sulfonyl]amino}-6′,6′-dioxido-1′,4′,17′-trioxo-2-vinyloctadecahydro-7′H-spiro[cyclopropane-1,3′-pyrrolo[2,1-g][1,2,5,8,19]thiatetraazacyclononadecin]-20′-yl 5-(dimethylamino)-1,3-dihydro-2H-isoindole-2-carboxylate, 0.3 mL (4.4 mmol) 2-mercapto-ethanol and 0.7 mL (4.4 mmol) DBU in 2 mL acetonitrile is stirred at rt for 5 h. The mixture is partitioned between EtOAc and water. The organic layer is washed with water, dried over Na₂SO₄ and concentrated to give the crude product which is used in the next step without further purification.

LC MS (method E) t_{R}=1.806 min, M+H=688.1

### Example 34

### (1R,2S,2′R,6′S,24a′S)-6′-amino-17′-fluoro-19′,19′-dioxido-5′, 21′,24′-trioxo-2-vinyl-1′,2′,3′,5′,6′,7′,8′,9′,10′,11′,12′,13′,14′,20′,21′,23′,24′,24a′-octadecahydrospiro[cyclopropane-1,22′-pyrrolo[2,1-g][1,2,5,8,18]benzothiatetraazacycloicosin]-2′-yl 4-fluoro-1,3-dihydro-2H-isoindole-2-carboxylate

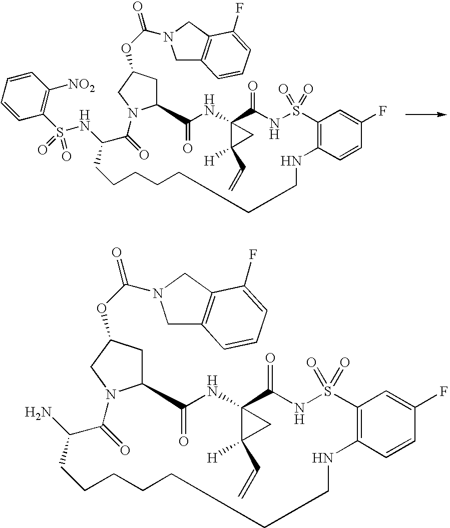

To a solution of 2.24 g (2.45 mmol) (1R,2S,2′R,6′S,24a′S)-17′-fluoro-6′-{[(2-nitrophenyl)-sulfonyl]amino}-19′,19′-dioxido-5′,21′,24′-trioxo-2-vinyl-1′,2′,3′,5′,6′,7′,8′,9′,10′,11′,12′,13′, 14′,20′,21′,23′,24′,24a′-octadecahydrospiro[cyclopropane-1,22′-pyrrolo[2,1-g][1,2,5,8,18]benzothiatetraazacycloicosin]-2′-yl 4-fluoro-1,3-dihydro-2H-isoindole-2-carboxylate (prepared analogously as described starting from ethyl (2S)-2-{[(2-nitrophenyl)sulfonyl]-amino}dec-9-enoate) in 230 mL acetonitrile is added at rt 1.85 mL (12.3 mmol) DBU followed by 1.9 mL (27 mmol) 2-mercaptoethanole. After 90 min the reaction mixture is concentrated, aq. bicarbonate is added and extracted with DCM. The organic layer is dried over Na₂SO₄, concentrated in vacuo and purified by FC on silica (eluent: DCM/MeOH 19:1→9:1).

MS (method): M+=729.2

HPLC (method) t_{R}=4.60 min

The following compounds (Table 1) can be prepared according to one of the methods described above.

TABLE 1

| Structure | Name | mass | t_{R} (min); mass | t_{R} (min) |
|---|---|---|---|---|
| 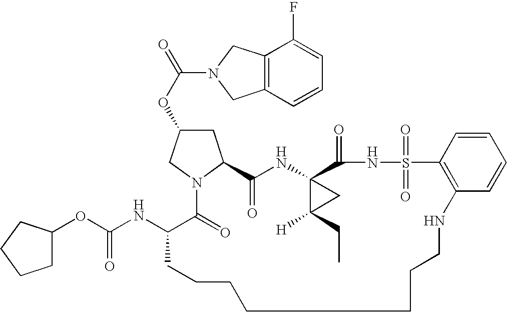 | Example 35: 4-Fluoro-1,3-dihydro-isoindole-2- carboxylic acid (8S,10R,14S)-14-cyclopentyloxy carbonyl-amino-5-[(1R,2S)-1-carbonylamino-2- ethyl-cyclopropyl]-2,2,4,7,13-pentaoxo-2Λ*6*- thia-3,6,12,22-tetraaza- tricyclo[21.4.0.0*8,12*]heptacosa-1(23),24,26- trien-10-yl ester | M + 1 = 825 MS method D | | 6.04 HPLC method A |
| 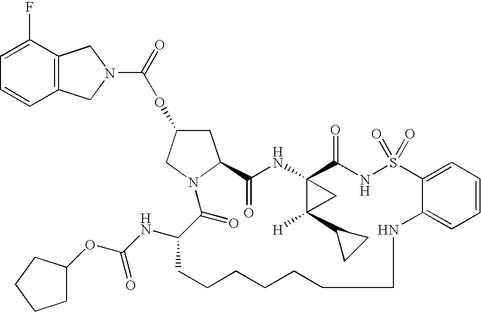 | Example 36: (1R,2S,2′R,6′S,24a′S)-6′- {[(cyclopentyloxy)carbonyl]amino}-2- cyclopropyl-19′,19′-dioxido-5′,21′,24′-trioxo- 1′,2′,3′,5′,6′,7′,8′,9′,10′,11′,12′,13′,14′,20′,21′,23′, 24′,24a′-octadecahydrospiro[cyclopropane- 1,22′-pyrrolo[2,1-g] [1,2,5,8,18]benzothiatetraazacycloicosin]-2′-yl 4-fluoro-1,3-dihydro-2H-isoindole-2-carboxylate | | M+ = 837.2 MS method D | 5.987 HPLC method A |
| 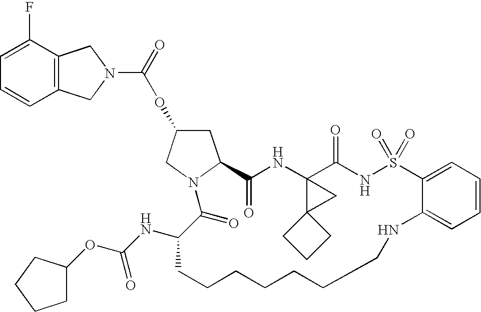 | Example 37: (2″R,6″S,24a″S)-6″- {[(cyclopentyloxy)carbonyl]amino}-19″,19″- dioxido-5″,21″,24″-trioxo- 1″,2″,3″,5″,6″,7″,8″,9″,10″,11″,12″,13″,14″, 20″,21″,23″,24″,24a″- octadecahydrodispiro[cyclobutane-1,1′- cyclopropane-2′,22″-pyrrolo[2,1- g][1,2,5,8,18]benzothiatetraazacycloicosin]-2″-yl 4-fluoro-1,3-dihydro-2H-isoindole-2-carboxylate (2 Diastereoisomers) | M+ = 837.2 MS method D | | 5.97 HPLC method A |
| 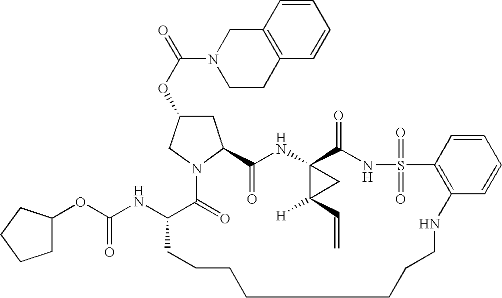 | Example 38: (1R,2S,2′R,6′S,24a′S)-6′- {[(cyclopentyloxy)carbonyl]amino}-19′,19′- dioxido-5′,21′,24′-trioxo-2-vinyl-1′,2′,3′,5′,6′,7′,8′, 9′,10′,11′,12′,13′,14′,20′,21′,23′,24′,24a′-octadeca- hydro-spiro[cyclopropane-1,22′-pyrrolo[2,1- g][1,2,5,8,18]benzothiatetraazacycloicosin]-2′-yl 3,4-dihydroisoquinoline-2(1H)-carboxylate | | 4.863; M − H = 817.3 LC MS method E | 4.516 HPLC method C |
| 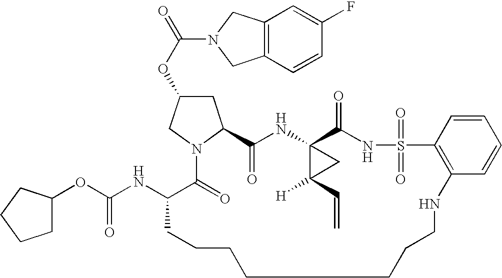 | Example 39: (1R,2S,2′R,6′S,24a′S)-6′- {[(cyclopentyloxy)carbonyl]amino}-19′,19′- dioxido-5′,21′,24′-trioxo-2-vinyl-1′,2′,3′,5′,6′,7′,8′, 9′,10′,11′,12′,13′,14′,20′,21′,23′,24′,24a′-octadeca- hydrospiro[cyclopropane-1,22′-pyrrolo[2,1- g][1,2,5,8,18]benzothiatetraazacycloicosin]-2′-yl 5-fluoro-1,3-dihydro-2H-isoindole-2-carboxylate | | 4.673; M − H = 821.2 LC MS method E | 4.426 HPLC method C |
| 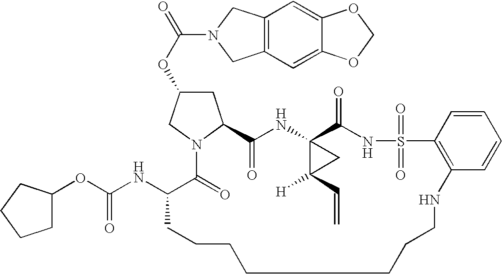 | Example 40: (1R,2S,2′R,6′S,24a′S)-6′- {[(cyclopentyloxy)carbonyl]amino}-19′,19′- dioxido-5′,21′,24′-trioxo-2-vinyl-1′,2′,3′,5′,6′,7′, 8′,9′,10′,11′,12′,13′,14′,20′,21′,23′,24′,24a′- octadecahydrospiro[cyclopropane-1,22′- pyrrolo[2,1-g][1,2,5,8,18]benzothiatetra- azacycloicosin]-2′-yl 5,7-dihydro-6H- [1,3]dioxolo[4,5-f]isoindole-6-carboxylate | | 4.663; M − H = 861.3 LC MS method E | 4.438 HPLC method C |
| 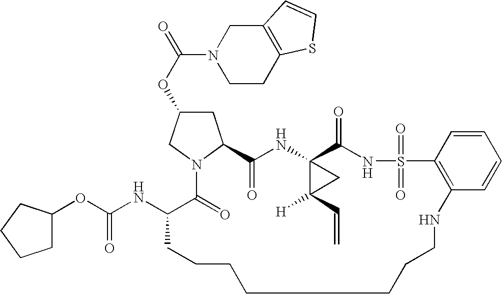 | Example 41: (1R,2S,2′R,6′S,24a′S)-6′- {[(cyclopentyloxy)carbonyl]amino}-19′,19′- dioxido-5′,21′,24′-trioxo-2-vinyl-1′,2′,3′,5′,6′,7′, 8′,9′,10′,11′,12′,13′,14′,20′,21′,23′,24′,24a′- octadecahydrospiro[cyclopropane-1,22′- pyrrolo[2,1-g][1,2,5,8,18]benzothiatetra- azacycloicosin]-2′-yl 6,7-dihydrothieno[3,2- c]pyridine-5(4H)-carboxylate | | 4.675; M + H = 826.3 LC MS method E | 4.499 HPLC method C |
| 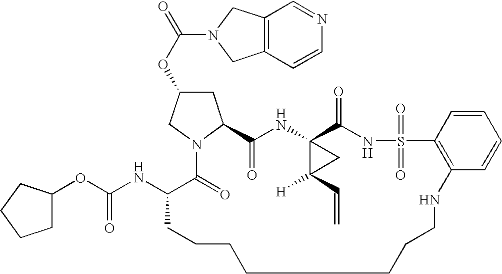 | Example 42: (1R,2S,2′R,6′S,24a′S)-6′- {[(cyclopentyloxy)carbonyl]amino}-19′,19′- dioxido-5′,21′,24′-trioxo-2-vinyl- 1′,2′,3′,5′,6′,7′,8′,9′,10′,11′,12′,13′,14′,20′,21′,23′, 24′,24a′-octadecahydrospiro[cyclopropane-1,22′- pyrrolo[2,1-g] [1,2,5,8,18]benzothiatetraazacycloicosin]-2′-yl 1,3-dihydro-2H-pyrrolo[3,4-c]pyridine-2- carboxylate | | 3.767; M − H = 804.3 LC MS method E | 3.492 HPLC method C |
| 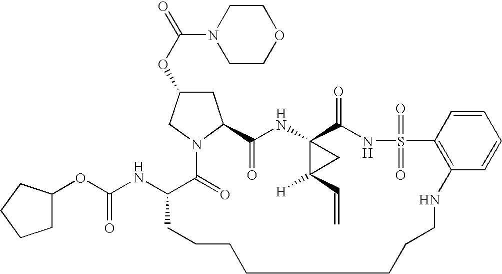 | Example 43: (1R,2S,2′R,6′S,24a′S)-6′- {[(cyclopentyloxy)carbonyl]amino}-19′,19′- dioxido-5′,21′,24′-trioxo-2-vinyl- 1′,2′,3′,5′,6′,7′,8′,9′,10′,11′,12′,13′,14′,20′,21′,23′, 24′,24a′-octadecahydrospiro[cyclopropane-1,22′- pyrrolo[2,1-g] [1,2,5,8,18]benzothiatetraazacycloicosin]-2′-yl morpholine-4-carboxylate | | 4.380; M + H = 773.3 LC MS method E | 4.157 HPLC method C |
| 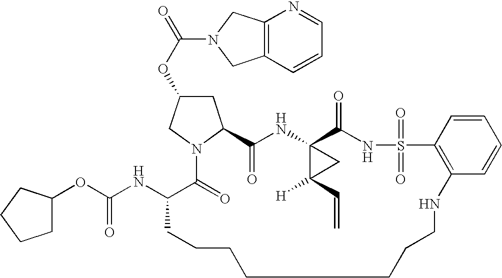 | Example 44: (1R,2S,2′R,6′S,24a′S)-6′- {[(cyclopentyloxy)carbonyl]amino}-19′,19′- dioxido-5′,21′,24′-trioxo-2-vinyl- 1′,2′,3′,5′,6′,7′,8′,9′,10′,11′,12′,13′,14′,20′,21′,23′, 24′,24a′-octadecahydrospiro[cyclopropane-1,22′- pyrrolo[2,1-g] [1,2,5,8,18]benzothiatetraazacycloicosin]-2′-yl 5,7-dihydro-6H-pyrrolo[3,4-b]pyridine-6- carboxylate | | 4.296; M − H = 804.3 LC MS method E | 3.774 HPLC method C |
| 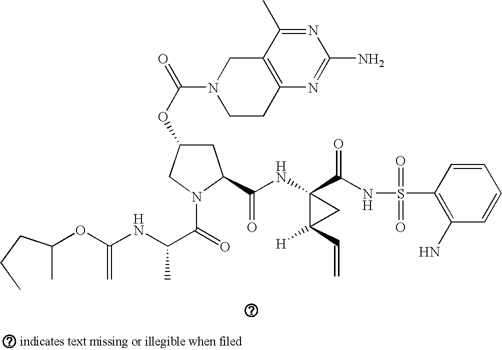 | Example 45: (1R,2S,2′R,6′S,24a′S)-6′- {[(cyclopentyloxy)carbonyl]amino}-19′,19′- dioxido-5′,21′,24′-trioxo-2-vinyl-1′,2′,3′,5′,6′,7′, 8′,9′,10′,11′,12′,13′,14′,20′,21′,23′,24′,24a′-octa- decahydrospiro[cyclopropane-1,22′-pyrrolo[2,1- g][1,2,5,8,18]benzothiatetraazacycloicosin]-2′-yl 2-amino-4-methyl-7,8-dihydropyrido[4,3- d]pyrimidine-6(5H)-carboxylate | | 3.897; M + H = 851.3 LC MS method E | 3.614 HPLC method C |
| 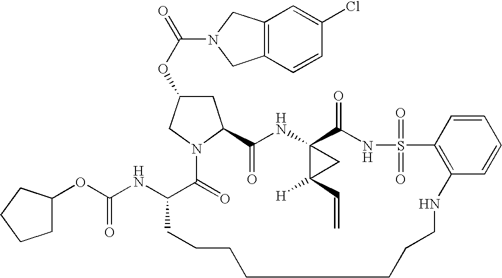 | Example 46: (1R,2S,2′R,6′S,24a′S)-6′- {[(cyclopentyloxy)carbonyl]amino}-19′,19′- dioxido-5′,21′,24′-trioxo-2-vinyl-1′,2′,3′,5′,6′,7′,8′, 9′,10′,11′,12′,13′,14′,20′,21′,23′,24′,24a′-octadeca- hydrospiro[cyclopropane-1,22′-pyrrolo[2,1- g][1,2,5,8,18]benzothiatetraazacycloicosin]-2′-yl 5-chloro-1,3-dihydro-2H-isoindole-2-carboxylate | | 4.850; M − H = 837.3 LC MS method E | 4.546 HPLC method C |
| 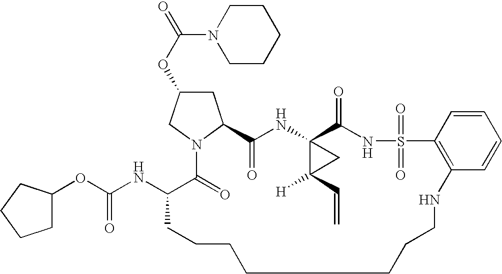 | Example 47: (1R,2S,2′R,6′S,24a′S)-6′- {[(cyclopentyloxy)carbonyl]amino}-19′,19′- dioxido-5′,21′,24′-trioxo-2-vinyl-1′,2′,3′,5′,6′,7′,8′, 9′,10′,11′,12′,13′,14′,20′,21′,23′,24′,24a′-octadeca- hydrospiro[cyclopropane-1,22′-pyrrolo[2,1- g][1,2,5,8,18]benzothiatetraazacycloicosin]-2′-yl piperidine-1-carboxylate | | 4.645; M + H = 771.3 LC MS method E | 4.439 HPLC method C |
| 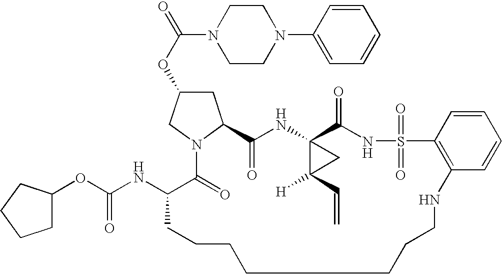 | Example 48: (1R,2S,2′R,6′S,24a′S)-6′- {[(cyclopentyloxy)carbonyl]amino}-19′,19′- dioxido-5′,21′,24′-trioxo-2-vinyl-1′,2′,3′,5′,6′,7′,8′, 9′,10′,11′,12′,13′,14′,20′,21′,23′,24′,24a′-octadeca- hydrospiro[cyclopropane-1,22′-pyrrolo[2,1- g][1,2,5,8,18]benzothiatetraazacycloicosin]-2′-yl 4-phenylpiperazine-1-carboxylate | | 4.761; M + H = 849.3 LC MS method E | 4.274 HPLC method C |
| 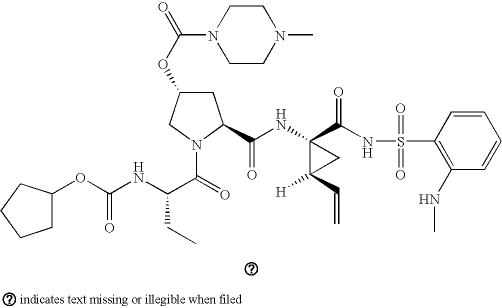 | Example 49: (1R,2S,2′R,6′S,24a′S)-6′- {[(cyclopentyloxy)carbonyl]amino}-19′,19′- dioxido-5′,21′,24′-trioxo-2-vinyl-1′,2′,3′,5′,6′,7′,8′, 9′,10′,11′,12′,13′,14′,20′,21′,23′,24′,24a′-octadeca- hydrospiro[cyclopropane-1,22′-pyrrolo[2,1- g][1,2,5,8,18]benzothiatetraazacycloicosin]-2′-yl 4-methylpiperazine-1-carboxylate | | 3.307; M − H = 784.3 LC MS method E | 3.534 HPLC method C |
| 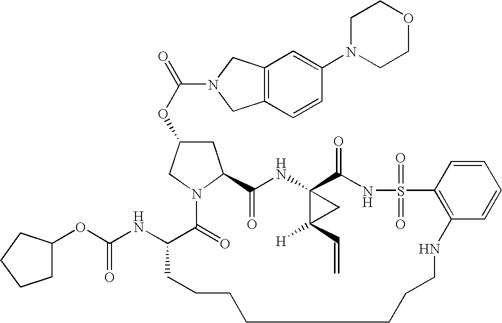 | Example 50: (1R,2S,2′R,6′S,24a′S)-6′- {[(cyclopentyloxy)carbonyl]amino}-19′,19′- dioxido-5′,21′,24′-trioxo-2-vinyl-1′,2′,3′,5′,6′,7′,8′, 9′,10′,11′,12′,13′,14′,20′,21′,23′,24′,24a′-octadeca- hydrospiro[cyclopropane-1,22′-pyrrolo[2,1-g] [1,2,5,8,18]benzothiatetraazacycloicosin]-2′-yl 5- morpholin-4-yl-1,3-dihydro-2H-isoindole-2- carboxylate | | 4.525; M + H = 891.4 LC MS method E | 4.078 HPLC method C |
| 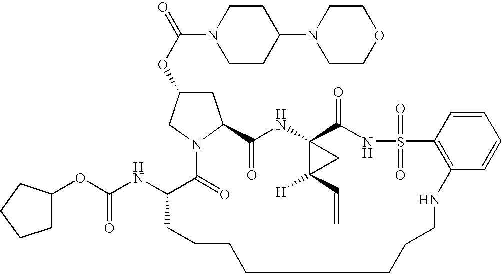 | Example 51: (1R,2S,2′R,6′S,24a′S)-6′- {[(cyclopentyloxy)carbonyl]amino}-19′,19′- dioxido-5′,21′,24′-trioxo-2-vinyl-1′,2′,3′,5′,6′,7′,8′, 9′,10′,11′,12′,13′,14′,20′,21′,23′,24′,24a′-octadeca- hydrospiro[cyclopropane-1,22′-pyrrolo[2,1- g][1,2,5,8,18]benzothiatetraazacycloicosin]-2′-yl 4-morpholin-4-ylpiperidine-1-carboxylate | | 3.350; M + H = 857.3 LC MS method E | 3.590 HPLC method C |
| 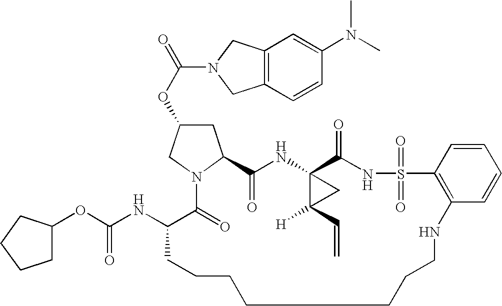 | Example 52: (1R,2S,2′R,6′S,24a′S)-6′- {[(cyclopentyloxy)carbonyl]amino}-19′,19′- dioxido-5′,21′,24′-trioxo-2-vinyl-1′,2′,3′,5′,6′,7′,8′, 9′,10′,11′,12′,13′,14′,20′,21′,23′,24′,24a′-octadeca- hydrospiro[cyclopropane-1,22′-pyrrolo[2,1- g][1,2,5,8,18]benzothiatetraazacycloicosin]-2′-yl 5-(dimethylamino)-1,3-dihydro-2H-isoindole-2- carboxylate | | 4.362; MH = 849.3 LC MS method E | 3.655 HPLC method C |
| 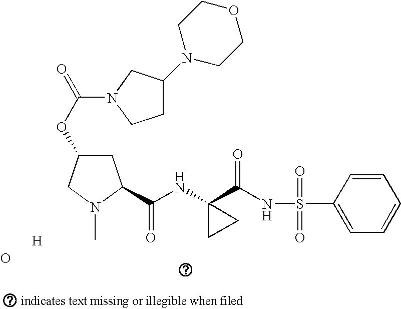 | Example 53: (1R,2S,2′R,6′S,24a′S)-6′- {[(cyclopentyloxy)carbonyl]amino}-19′,19′- dioxido-5′,21′,24′-trioxo-2-vinyl-1′,2′,3′,5′,6′,7′,8′, 9′,10′,11′,12′,13′,14′,20′,21′,23′,24′,24a′-octadeca- hydrospiro[cyclopropane-1,22′-pyrrolo[2,1- g][1,2,5,8,18]benzothiatetraazacycloicosin]-2′-yl 3-morpholin-4-ylpyrrolidine-1-carboxylate | | 3.240; M − H = 841.3 LC MS method E | 3.572 HPLC method C |
| 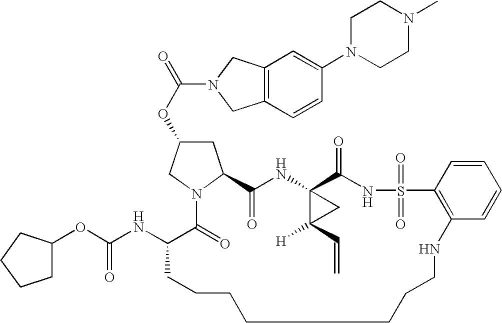 | Example 54: (1R,2S,2′R,6′S,24a′S)-6′- {[(cyclopentyloxy)carbonyl]amino}-19′,19′- dioxido-5′,21′,24′-trioxo-2-vinyl-1′,2′,3′,5′,6′,7′,8′, 9′,10′,11′,12′,13′,14′,20′,21′,23′,24′,24a′-octadeca- hydrospiro[cyclopropane-1,22′-pyrrolo[2,1- g][1,2,5,8,18]benzothiatetraazacycloicosin]-2′-yl 5-(4-methylpiperazin-1-yl)-1,3-dihydro-2H- isoindole-2-carboxylate | | 3.290; M + H = 904.3 LC MS method E | 3.609 HPLC method C |
| 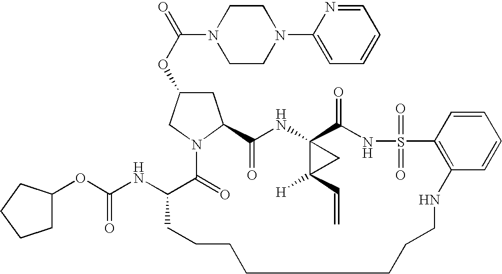 | Example 55: (1R,2S,2′R,6′S,24a′S)-6′- {[(cyclopentyloxy)carbonyl]amino}-19′,19′- dioxido-5′,21′,24′-trioxo-2-vinyl-1′,2′,3′,5′,6′,7′,8′, 9′,10′,11′,12′,13′,14′,20′,21′,23′,24′,24a′-octadeca- hydrospiro[cyclopropane-1,22′-pyrrolo[2,1- g][1,2,5,8,18]benzothiatetraazacycloicosin]-2′-yl 4-pyridin-2-ylpiperazine-1-carboxylate | | 3.773; M + H = 850.3 LC MS method E | 3.608 HPLC method C |
| 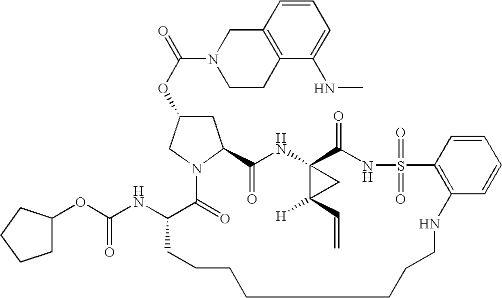 | Example 56: (1R,2S,2′R,6′S,24a′S)-6′- {[(cyclopentyloxy)carbonyl]amino}-19′,19′- dioxido-5′,21′,24′-trioxo-2-vinyl-1′,2′,3′,5′,6′,7′,8′, 9′,10′,11′,12′,13′,14′,20′,21′,23′,24′,24a′-octadeca- hydrospiro[cyclopropane-1,22′-pyrrolo[2,1- g][1,2,5,8,18]benzothiatetraazacycloicosin]-2′-yl 5-(methylamino)-3,4-dihydroisoquinoline-2(1H)- carboxylate | | 4.619; M + H = 849.4 LC MS method E | 3.828 HPLC method C |
| 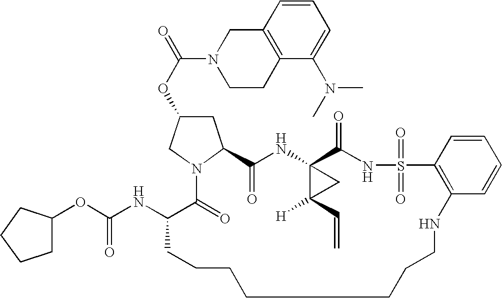 | Example 57: (1R,2S,2′R,6′S,24a′S)-6′- {[(cyclopentyloxy)carbonyl]amino}-19′,19′- dioxido-5′,21′,24′-trioxo-2-vinyl- 1′,2′,3′,5′,6′,7′,8′,9′,10′,11′,12′,13′,14′,20′,21′,23′, 24′,24a′-octadecahydrospiro[cyclopropane- 1,22′-pyrrolo[2,1-g] [1,2,5,8,18]benzothiatetraazacycloicosin]-2′-yl 5-(dimethylamino)-3,4-dihydroisoquinoline- 2(1H)-carboxylate | | 4.185; M + H = 863.3 LC MS method E | 3.659 HPLC method C |
| 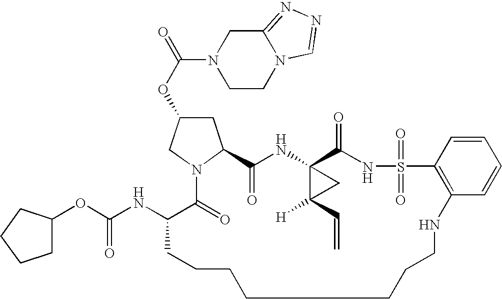 | Example 58: (1R,2S,2′R,6′S,24a′S)-6′- {[(cyclopentyloxy)carbonyl]amino}-19′,19′- dioxido-5′,21′,24′-trioxo-2-vinyl- 1′,2′,3′,5′,6′,7′,8′,9′,10′,11′,12′,13′,14′,20′,21′,23′, 24′,24a′-octadecahydrospiro[cyclopropane- 1,22′-pyrrolo[2,1-g] [1,2,5,8,18]benzothiatetraazacycloicosin]-2′-yl 5,6-dihydro[1,2,4]triazolo[4,3-a]pyrazine-7(8H)- carboxylate | | 4.205; M + H = 810.3 LC MS method E | 4.059 HPLC method C |
| 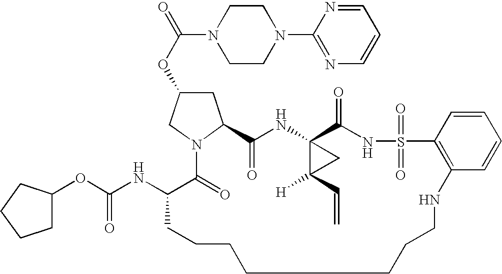 | Example 59: (1R,2S,2′R,6′S,24a′S)-6′- {[(cyclopentyloxy)carbonyl]amino}-19′,19′- dioxido-5′,21′,24′-trioxo-2-vinyl- 1′,2′,3′,5′,6′,7′,8′,9′,10′,11′,12′,13′,14′,20′,21′,23′, 24′,24a′-octadecahydrospiro[cyclopropane- 1,22′-pyrrolo[2,1-g] [1,2,5,8,18]benzothiatetraazacycloicosin]-2′-yl 4-pyrimidin-2-ylpiperazine-1-carboxylate | | 4.512; M + H = 851.3 LC MS method E | 4.220 HPLC method C |
| 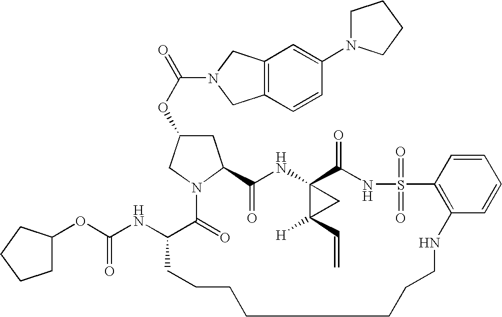 | Example 60: (1R,2S,2′R,6′S,24a′S)-6′- {[(cyclopentyloxy)carbonyl]amino}-19′,19′- dioxido-5′,21′,24′-trioxo-2-vinyl- 1′,2′,3′,5′,6′,7′,8′,9′,10′,11′,12′,13′,14′,20′,21′,23′, 24′,24a′-octadecahydrospiro[cyclopropane- 1,22′-pyrrolo[2,1-g] [1,2,5,8,18]benzothiatetraazacycloicosin]-2′-yl 5-pyrrolidin-1-yl-1,3-dihydro-2H-isoindole-2- carboxylate | | 4.849; M + H = 875.3 LC MS method E | 4.344 HPLC method C |
| 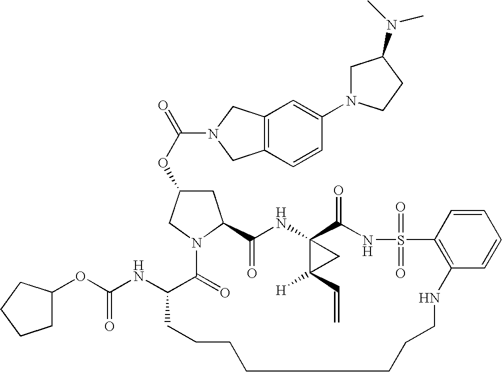 | Example 61: (1R,2S,2′R,6′S,24a′S)-6′- {[(cyclopentyloxy)carbonyl]amino}-19′,19′- dioxido-5′,21′,24′-trioxo-2-vinyl- 1′,2′,3′,5′,6′,7′,8′,9′,10′,11′,12′,13′,14′,20′,21′,23′, 24′,24a′-octadecahydrospiro[cyclopropane- 1,22′-pyrrolo[2,1-g] [1,2,5,8,18]benzothiatetraazacycloicosin]-2′-yl 5-[(3S)-3-(dimethylamino)pyrrolidin-1-yl]-1,3- dihydro-2H-isoindole-2-carboxylate | | 3.424; M − H = 916.3 LC MS method E | 3.601 HPLC method C |
| 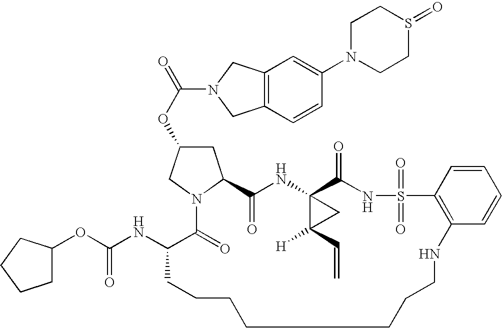 | Example 62: (1R,2S,2′R,6′S,24a′S)-6′- {[(cyclopentyloxy)carbonyl]amino}-19′,19′- dioxido-5′,21′,24′-trioxo-2-vinyl- 1′,2′,3′,5′,6′,7′,8′,9′,10′,11′,12′,13′,14′,20′,21′,23′, 24′,24a′-octadecahydrospiro[cyclopropane- 1,22′-pyrrolo[2,1-g] [1,2,5,8,18]benzothiatetraazacycloicosin]-2′-yl 5-(1-oxidothiomorpholin-4-yl)-1,3-dihydro-2H- isoindole-2-carboxylate | | LC MS method E | 4.166 HPLC method C |
| 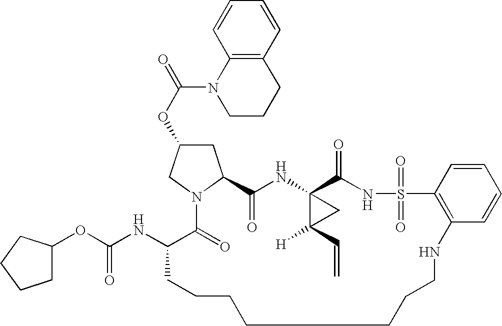 | Example 63: (1R,2S,2′R,6′S,24a′S)-6′- {[(cyclopentyloxy)carbonyl]amino}-19′,19′- dioxido-5′,21′,24′-trioxo-2-vinyl-1′,2′,3′,5′,6′,7′,8′, 9′,10′,11′,12′,13′,14′,20′,21′,23′,24′,24a′-octadeca- hydrospiro[cyclopropane-1,22′-pyrrolo[2,1- g][1,2,5,8,18]benzothiatetraazacycloicosin}-2′-yl 3,4-dihydroquinoline-1(2H)-carboxylate | | 4.776; M + H = 819.3 LC MS method E | 4.591 HPLC method C |
| 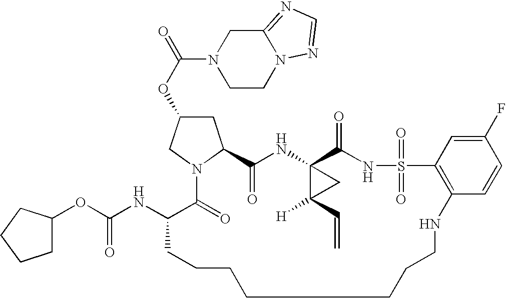 | Example 64: (1R,2S,2′R,6′S,24a′S)-6′- {[(cyclopentyloxy)carbonyl]amino}-17′-fluoro- 19′,19′-dioxido-5′,21′,24′-trioxo-2-vinyl- 1′,2′,3′,5′,6′,7′,8′,9′,10′,11′,12′,13′,14′,20′,21′,23′, 24′,24a′-octadecahydrospiro[cyclopropane-1,22′- pyrrolo[2,1-g][1,2,5,8,18]benzothiatetraaza- cycloicosin]-2′-yl 5,6-dihydro[1,2,4]triazolo[1,5- a]pyrazine-7(8H)-carboxylate | | 4.101; M + H = 828.3 LC MS method E | 4.176 HPLC method C |
| 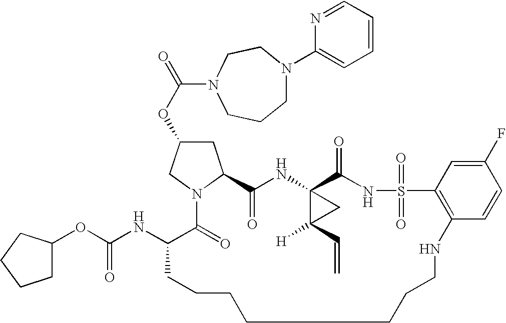 | Example 65: (1R,2S,2′R,6′S,24a′S)-6′- {[(cyclopentyloxy)carbonyl]amino}-17′-fluoro- 19′,19′-dioxido-5′,21′,24′-trioxo-2-vinyl-1′,2′,3′,5′, 6′,7′,8′,9′,10′,11′,12′,13′,14′,20′,21′,23′,24′,24a′- octadecahydrospiro[cyclopropane-1,22′- pyrrolo[2,1-g][1,2,5,8,18]benzothiatetra- azacycloicosin]-2′-yl 4-pyridin-2-yl-1,4- diazepane-1-carboxylate | | 3.506; M + H = 882.3 LC MS method E | 3.792 HPLC method C |
| 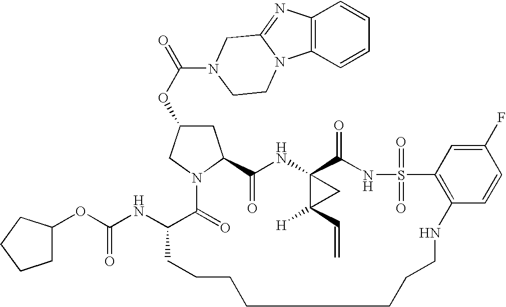 | Example 66: (1R,2S,2′R,6′S,24a′S)-6′- {[(cyclopentyloxy)carbonyl]amino}-17′-fluoro- 19′,19′-dioxido-5′,21′,24′-trioxo-2-vinyl-1′,2′,3′,5′, 6′,7′,8′,9′,10′,11′,12′,13′,14′,20′,21′,23′,24′,24a′- octadecahydrospiro[cyclopropane-1,22′-pyrrolo [2,1-g][1,2,5,8,18]benzothiatetraazacycloicosin]- 2′-yl 3,4-dihydropyrazino[1,2-a]benzimidazole- 2(1H)-carboxylate | | 3.988; M + H = 878.3 LC MS method E | 3.872 HPLC method C |
| 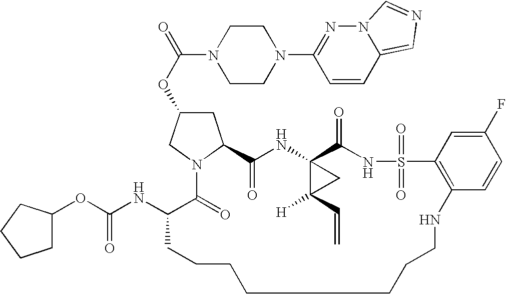 | Example 67: (1R,2S,2′R,6′S,24a′S)-6′- {[(cyclopentyloxy)carbonyl]amino}-17′-fluoro- 19′,19′-dioxido-5′,21′,24′-trioxo-2-vinyl-1′,2′,3′, 5′,6′,7′,8′,9′,10′,11′,12′,13′,14′,20′,21′,23′,24′,24a′- octadecahydrospiro[cyclopropane-1,22′-pyrrolo [2,1-g][1,2,5,8,18]benzothiatetraazacycloicosin]- 2′-yl 4-imidazo[1,5-b]pyridazin-2-ylpiperazine- 1-carboxylate | | 3.473; M + H = 907.3 LC MS method E | 3.663 HPLC method C |
| 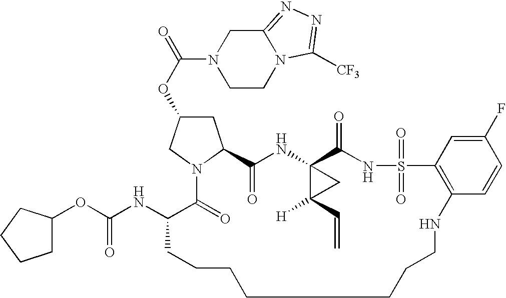 | Example 68: (1R,2S,2′R,6′S,24a′S)-6′- {[(cyclopentyloxy)carbonyl]amino}-17′-fluoro- 19′,19′-dioxido-5′,21′,24′-trioxo-2-vinyl-1′,2′,3′,5′, 6′,7′,8′,9′,10′,11′,12′,13′,14′,20′,21′,23′,24′,24a′- octadecahydrospiro[cyclopropane-1,22′-pyrrolo [2,1-g][1,2,5,8,18]benzothiatetraazacycloicosin]- 2′-yl 3-(trifluoromethyl)-5,6-dihydro[1,2,4] triazolo[4,3-a]pyrazine-7(8H)-carboxylate | | 4.418; M + H = 896.2 LC MS method E | 4.330 HPLC method C |
| 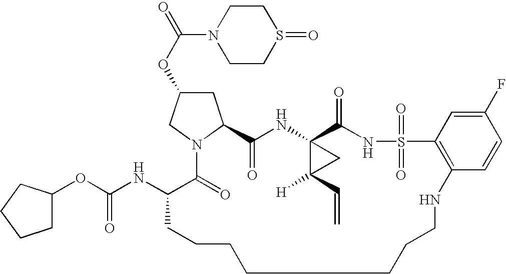 | Example 69: (1R,2S,2′R,6′S,24a′S)-6′- {[(cyclopentyloxy)carbonyl]amino}-17′-fluoro- 19′,19′-dioxido-5′,21′,24′-trioxo-2-vinyl-1′,2′,3′,5′, 6′,7′,8′,9′,10′,11′,12′,13′,14′,20′,21′,23′,24′,24a′- octadecahydrospiro[cyclopropane-1,22′-pyrrolo [2,1-g][1,2,5,8,18]benzothiatetraaza- cycloicosin]-2′-yl thiomorpholine-4-carboxylate 1-oxide | | 4.076; M − H = 821.2 LC MS method E | 4.038 HPLC method C |
| 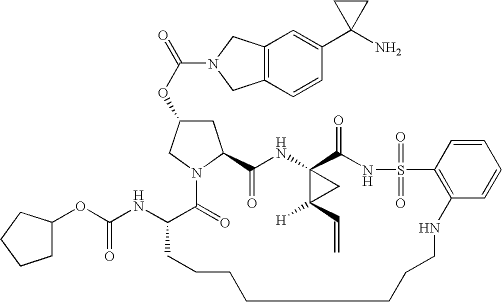 | Example 70: (1R,2S,2′R,6′S,24a′S)-6′- {[(cyclopentyloxy)carbonyl]amino}-19′,19′- dioxido-5′,21′,24′-trioxo-2-vinyl-1′,2′,3′,5′,6′,7′, 8′,9′,10′,11′,12′,13′,14′,20′,21′,23′,24′,24a′- octadecahydrospiro[cyclopropane-1,22′-pyrrolo [2,1-g][1,2,5,8,18]benzothiatetraazacycloicosin]- 2′-yl 5-(1-aminocyclopropyl)-1,3-dihydro-2H- isoindole-2-carboxylate | | 3.396; M + H = 879.3 LC MS method E | 3.610 HPLC method C |
| 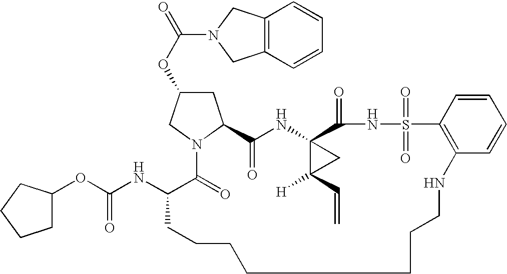 | Example 71: (1R,2S,2′R,6′S,24a′S)-6′- {[(cyclopentyloxy)carbonyl]amino}-19′,19′- dioxido-5′,21′,24′-trioxo-2-vinyl-1′,2′,3′,5′,6′,7′, 8′,9′,10′,11′,12′,13′,14′,20′,21′,23′,24′,24a′- octadecahydrospiro[cyclopropane-1,22′-pyrrolo [2,1-g][1,2,5,8,18]benzothiatetraazacycloicosin]- 2′-yl 1,3-dihydro-2H-isoindole-2-carboxylate | | 4.709; M − H = 803.3 LC MS method E | 4.394 HPLC method C |
| 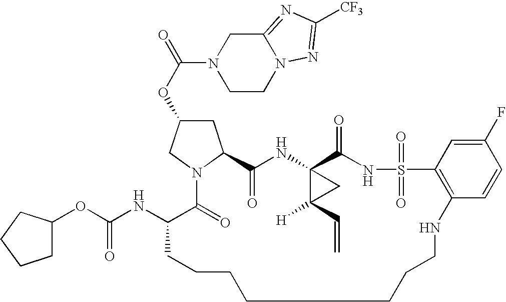 | Example 72: (1R,2S,2′R,6′S,24a′S)-6′- {[(cyclopentyloxy)carbonyl]amino}-17′-fluoro- 19′,19′-dioxido-5′,21′,24′-trioxo-2-vinyl-1′,2′,3′,5′, 6′,7′,8′,9′,10′,11′,12′,13′,14′,20′,21′,23′,24′,24a′- octadecahydrospiro[cyclopropane-1,22′-pyrrolo [2,1-g][1,2,5,8,18]benzothiatetraazacycloicosin]- 2′-yl 2-(trifluoromethyl)-5,6-dihydro[1,2,4] triazolo[1,5-a]pyrazine-7(8H)-carboxylate | | 4.557; M − H = 896.2 LC MS method E | 4.5 HPLC method C |
| 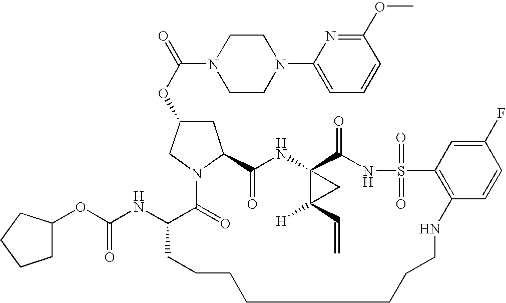 | Example 73: (1R,2S,2′R,6′S,24a′S)-6′- {[(cyclopentyloxy)carbonyl]amino}-17′-fluoro- 19′,19′-dioxido-5′,21′,24′-trioxo-2-vinyl-1′,2′,3′,5′, 6′,7′,8′,9′,10′,11′,12′,13′,14′,20′,21′,23′,24′,24a′- octadecahydrospiro[cyclopropane-1,22′- pyrrolo[2,1-g][1,2,5,8,18]benzothiatetra- azacycloicosin]-2′-yl 4-(6-methoxypyridin-2- yl)piperazine-1-carboxylate | | 4.670; M + H = 898.4 LC MS method E | 4.702 HPLC method C |
| 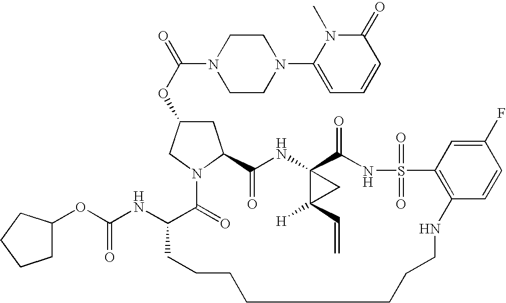 | Example 74: (1R,2S,2′R,6′S,24a′S)-6′- {[(cyclopentyloxy)carbonyl]amino}-17′-fluoro- 19′,19′-dioxido-5′,21′,24′-trioxo-2-vinyl-1′,2′,3′,5′, 6′,7′,8′,9′,10′,11′,12′,13′,14′,20′,21′,23′,24′,24a′- octadecahydrospiro[cyclopropane-1,22′-pyrrolo [2,1-g][1,2,5,8,18]benzothiatetraazacycloicosin]- 2′-yl 4-(1-methyl-6-oxo-1,6-dihydropyridin-2- yl)piperazine-1-carboxylate | | 4.124; M + H = 898.3 LC MS method E | 4.211 HPLC method C |
| 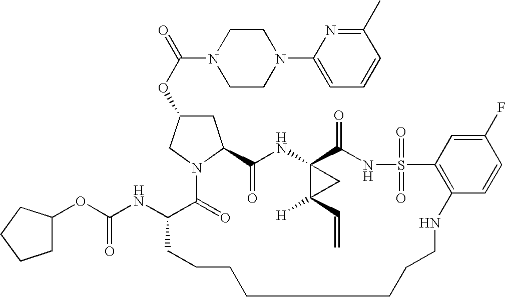 | Example 75: (1R,2S,2′R,6′S,24a′S)-6′- {[(cyclopentyloxy)carbonyl]amino}-17′-fluoro- 19′,19′-dioxido-5′,21′,24′-trioxo-2-vinyl-1′,2′,3′,5′, 6′,7′,8′,9′,10′,11′,12′,13′,14′,20′,21′,23′,24′,24a′- octadecahydrospiro[cyclopropane-1,22′-pyrrolo [2,1-g][1,2,5,8,18]benzothiatetraazacycloicosin]- 2′-y1 4-(6-methylpyridin-2-yl)piperazine-1- carboxylate | | 3.778; M + H = 882.3 LC MS method E | 3.721 HPLC method C |
| 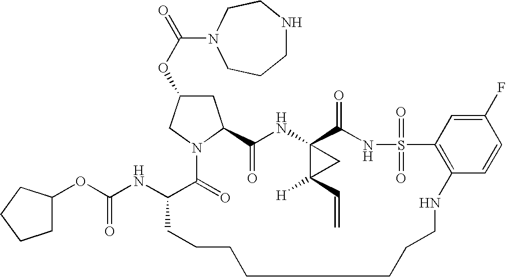 | Example 76: (1R,2S,2′R,6′S,24a′S)-6′- {[(cyclopentyloxy)carbonyl]amino}-17′-fluoro- 19′,19′-dioxido-5′,21′,24′-trioxo-2-vinyl-1′,2′,3′,5′, 6′,7′,8′,9′,10′,11′,12′,13′,14′,20′,21′,23′,24′,24a′- octadecahydrospiro[cyclopropane-1,22′-pyrrolo [2,1-g][1,2,5,8,18]benzothiatetraazacycloicosin]- 2′-yl 1,4-diazepane-1-carboxylate | | 3.234; M + H = 804.3 LC MS method E | HPLC method C |
| 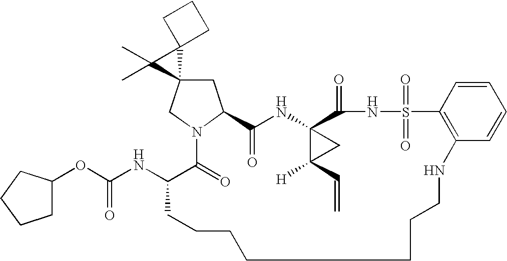 | Example 77: cyclopentyl [(2′R,2′′′S,6″S,22″R,24a″S)-3′,3′-dimethyl- 19″,19″-dioxido-5″,21″,24″-trioxo-2′′′-vinyl- 1″,5″,6″,7″,8″,9″,10″,11″,12″,13″,14″,20″,21″,23″, 24″,24a″-hexadecahydrotrispiro[cyclobutane- 1,1′-cyclopropane-2′,2″-pyrrolo[2,1- g][1,2,5,8,18]benzothiatetraazacycloicosine- 22″,1′′′-cyclopropan]-6″-yl]carbamate | | 5.227; M + H = 738.3 LC MS method E | 5.008 HPLC method C |
| 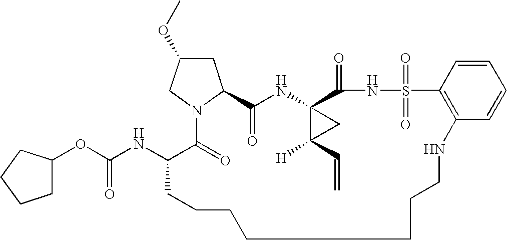 | Example 78: cyclopentyl [(1R,2S,2′R,6′S,24a′S)- 2′-methoxy-19′,19′-dioxido-5′,21′,24′-trioxo-2- vinyl-1′,2′,3′,5′,6′,7′,8′,9′,10′,11′,12′,13′,14′,20′,21′, 23′,24′,24a′-octadecahydrospiro[cyclopropane- 1,22′-pyrrolo[2,1-g][1,2,5,8,18]benzothiatetraaza- cycloicosin]-6′-yl]carbamate | | 4.539; M + H = 674.2 LC MS method E | 4.319 HPLC method C |
| 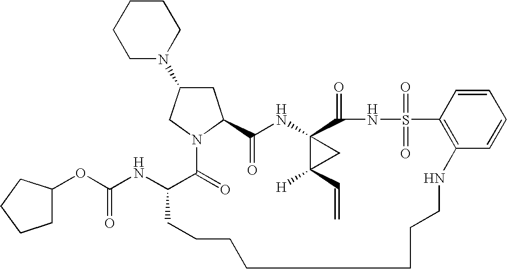 | Example 79: cyclopentyl [(1R,2S,2′R,6′S,24a′S)- 19′,19′-dioxido-5′,21′,24′-trioxo-2′-piperidin-1-yl- 2-vinyl-1′,2′,3′,5′,6′,7′,8′,9′,10′,11′,12′,13′,14′,20′, 21′,23′,24′,24a′-octadecahydrospiro [cyclopropane-1,22′-pyrrolo[2,1-g][1,2,5,8,18] benzothiatetraazacycloicosin]-6′-yl]carbamate | | 3.530; M − H = 725.3 LC MS method E | 3.596 HPLC method C |
| 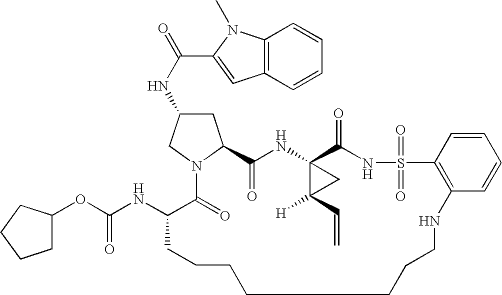 | Example 80: Cyclopentyl [(1R,2S,2′R,6′S,24a′S)- 2′-{[(1-methyl-1H-indol-2-yl)carbonyl]amino}- 19′,19′-dioxido-5′,21′,24′-trioxo-2-vinyl-1′,2′,3′,5′, 6′,7′,8′,9′,10′,11′,12′,13′,14′,20′,21′,23′,24′,24a′- octadecahydro-spiro[cyclopropane-1,22′-pyrrolo [2,1-g][1,2,5,8,18]benzothiatetraazacycloicosin]- 6′-yl]-carbamate | | 4.793; M + H = 817.3 LC MS method E | 4.607 HPLC method C |
| 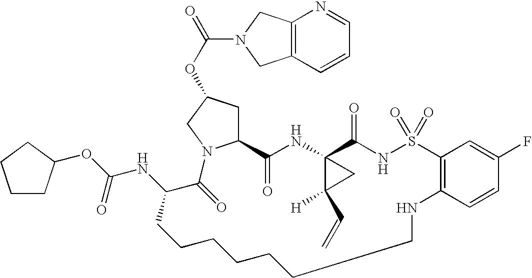 | Example 81: (1R,2S,2′R,6′S,24a′S)-6′- {[(cyclopentyloxy)carbonyl]amino}-17′-fluoro- 19′,19′-dioxido-5′,21′,24′-trioxo-2-vinyl-1′,2′,3′,5′, 6′,7′,8′,9′,10′,11′,12′,13′,14′,20′,21′,23′,24′,24a′- octadecahydrospiro[cyclopropane-1,22′-pyrrolo [2,1-g][1,2,5,8,18]benzothiatetraazacycloicosin]- 2′-yl 5,7-dihydro-6H-pyrrolo[3,4-b]pyridine-6- carboxylate | M+ = 824.2 MS method D | | 5.28 HPLC method A |
| 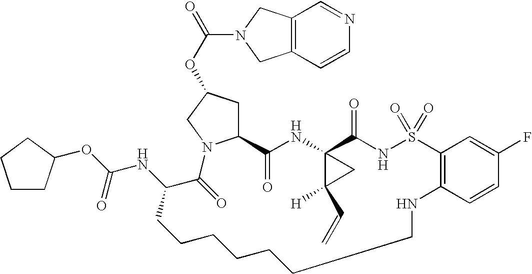 | Example 82: (1R,2S,2′R,6′S,24a′S)-6′- {[(cyclopentyloxy)carbonyl]amino}-17′-fluoro- 19′,19′-dioxido-5′,21′,24′-trioxo-2-vinyl-1′,2′,3′,5′, 6′,7′,8′,9′,10′,11′,12′,13′,14′,20′,21′,23′,24′,24a′- octadecahydrospiro[cyclopropane-1,22′-pyrrolo [2,1-g][1,2,5,8,18]benzothiatetraazacycloicosin]- 2′-yl 1,3-dihydro-2H-pyrrolo[3,4-c]pyridine-2- carboxylate | M+ = 824.2 MS method D | | 4.970 HPLC method A |
| 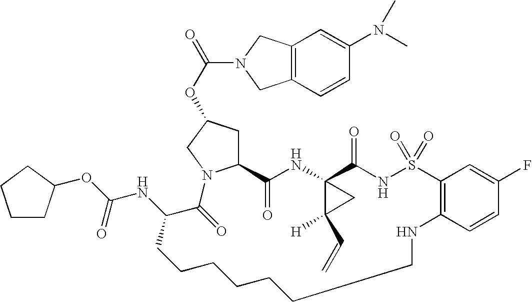 | Example 82: (1R,2S,2′R,6′S,24a′S)-6′- {[(cyclopentyloxy)carbonyl]amino}-17′-fluoro- 19′,19′-dioxido-5′,21′,24′-trioxo-2-vinyl- 1′,2′,3′,5′,6′,7′,8′,9′,10′,11′,12′,13′,14′,20′,21′,23′, 24′,24a′-octadecahydrospiro[cyclopropane- 1,22′-pyrrolo[2,1-g] [1,2,5,8,18]benzothiatetraazacycloicosin]-2′-yl 5-(dimethylamino)-1,3-dihydro-2H-isoindole-2- carboxylate | M+ = 866.2 MS method D | | 3.31 HPLC method A |
| 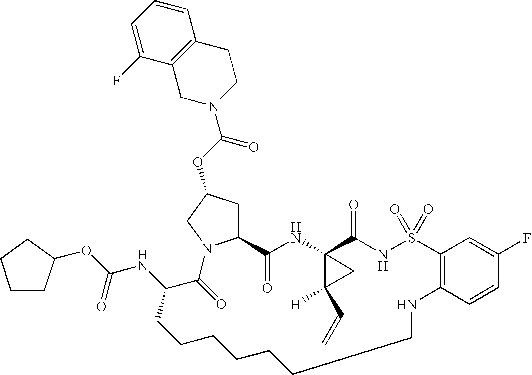 | Example 82: (1R,2S,2′R,6′S,24a′S)-6′- {[(cyclopentyloxy)carbonyl]amino}-17′-fluoro- 19′,19′-dioxido-5′,21′,24′-trioxo-2-vinyl- 1′,2′,3′,5′,6′,7′,8′,9′,10′,11′,12′,13′,14′,20′,21′,23′, 24′,24a′-octadecahydrospiro[cyclopropane- 1,22′-pyrrolo[2,1-g] [1,2,5,8,18]benzothiatetraazacycloicosin]-2′-yl 8-fluoro-3,4-dihydroisoquinoline-2(1H)- carboxylate | M+ = 855.2 MS method D | | 7.13 HPLC method A |
| 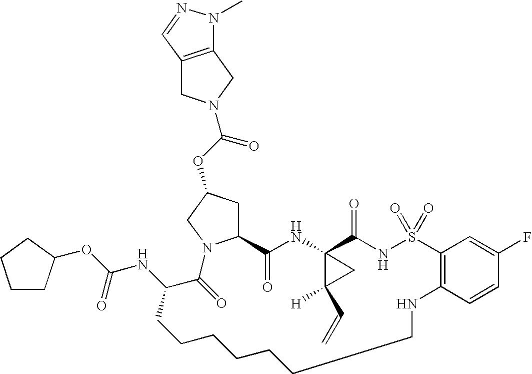 | Example 83: (1R,2S,2′R,6′S,24a′S)-6′- {[(cyclopentyloxy)carbonyl]amino}-17′-fluoro- 19′,19′-dioxido-5′,21′,24′-trioxo-2-vinyl- 1′,2′,3′,5′,6′,7′,8′,9′,10′,11′,12′,13′,14′,20′,21′,23′, 24′,24a′-octadecahydrospiro[cyclopropane- 1,22′-pyrrolo[2,1-g] [1,2,5,8,18]benzothiatetraazacycloicosin]-2′-yl 1-methyl-4,6-dihydropyrrolo[3,4-c]pyrazole- 5(1H)-carboxylate | M+ = 827.2 MS method D | | 6.23 HPLC method A |
| 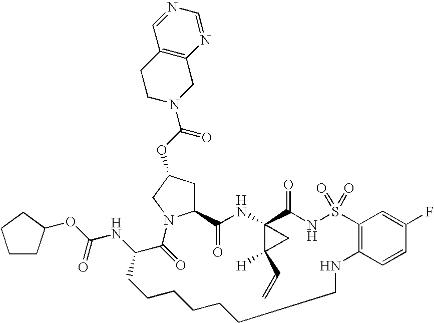 | Example 84: (1R,2S,2′R,6′S,24a′S)-6′- {[(cyclopentyloxy)carbonyl]amino}-17′-fluoro- 19′,19′-dioxido-5′,21′,24′-trioxo-2-vinyl- 1′,2′,3′,5′,6′,7′,8′,9′,10′,11′,12′,13′,14′,20′,21′,23′, 24′,24a′-octadecahydrospiro[cyclopropane- 1,22′-pyrrolo[2,1-g] [1,2,5,8,18]benzothiatetraazacycloicosin]-2′-yl 5,8-dihydropyrido[3,4-d]pyrimidine-7(6H)- carboxylate | M+ = 839.2 MS method D | | 6.27 HPLC method A |
| 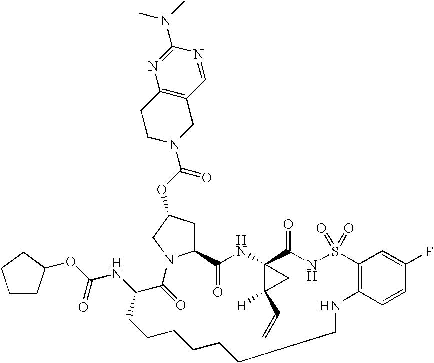 | Example 85: (1R,2S,2′R,6′S,24a′S)-6′- {[(cyclopentyloxy)carbonyl]amino}-17′-fluoro- 19′,19′-dioxido-5′,21′,24′-trioxo-2-vinyl- 1′,2′,3′,5′,6′,7′,8′,9′,10′,11′,12′,13′,14′,20′,21′,23′, 24′,24a′-octadecahydrospiro[cyclopropane- 1,22′-pyrrolo[2,1-g] [1,2,5,8,18]benzothiatetraazacycloicosin]-2′-yl 2-(dimethylamino)-7,8-dihydropyrido[4,3- d]pyrimidine-6(5H)-carboxylate | M+ = 882.2 MS method D | | 5.70 HPLC method A |
| 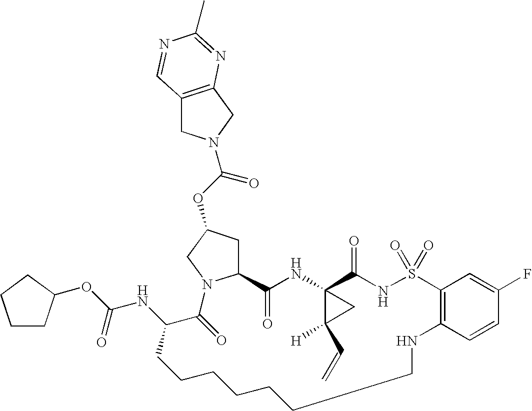 | Example 86: (1R,2S,2′R,6′S,24a′S)-6′- {[(cyclopentyloxy)carbonyl]amino}-17′-fluoro- 19′,19′-dioxido-5′,21′,24′-trioxo-2-vinyl- 1′,2′,3′,5′,6′,7′,8′,9′,10′,11′,12′,13′,14′,20′,21′,23′, 24′,24a′-octadecahydrospiro[cyclopropane- 1,22′-pyrrolo[2,1-g] [1,2,5,8,18]benzothiatetraazacycloicosin]-2′-yl 2-methyl-5,7-dihydro-6H-pyrrolo[3,4- d]pyrimidine-6-carboxylate | M+ = 839.2 MS method D | | 6.16 HPLC method A |
| 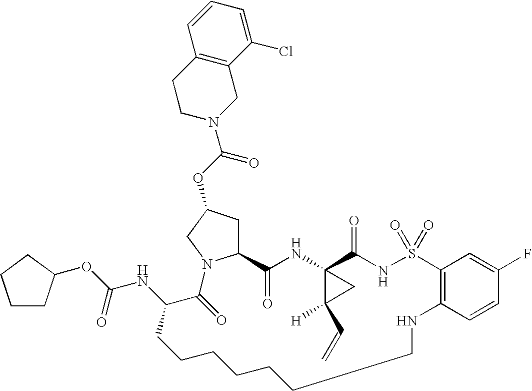 | Example 87: (1R,2S,2′R,6′S,24a′S)-6′- {[(cyclopentyloxy)carbonyl]amino}-17′-fluoro- 19′,19′-dioxido-5′,21′,24′-trioxo-2-vinyl- 1′,2′,3′,5′,6′,7′,8′,9′,10′,11′,12′,13′,14′,20′,21′,23′, 24′,24a′-octadecahydrospiro[cyclopropane- 1,22′-pyrrolo[2,1-g] [1,2,5,8,18]benzothiatetraazacycloicosin]-2′-yl 8-chloro-3,4-dihydroisoquinoline-2(1H)- carboxylate | M+ = 871.2 MS method D | | 7.27 HPLC method A |
| 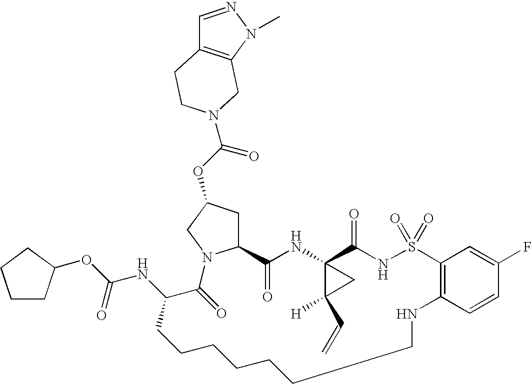 | Example 88: (1R,2S,2′R,6′S,24a′S)-6′- {[(cyclopentyloxy)carbonyl]amino}-17′-fluoro- 19′,19′-dioxido-5′,21′,24′-trioxo-2-vinyl- 1′,2′,3′,5′,6′,7′,8′,9′,10′,11′,12′,13′,14′,20′,21′,23′, 24′,24a′-octadecahydrospiro[cyclopropane- 1,22′-pyrrolo[2,1-g] [1,2,5,8,18]benzothiatetraazacycloicosin]-2′-yl 1-methyl-1,4,5,7-tetrahydro-6H-pyrazolo[3,4- c]pyridine-6-carboxylate | M+ = 841.2 MS method D | | 6.21 HPLC method A |
| 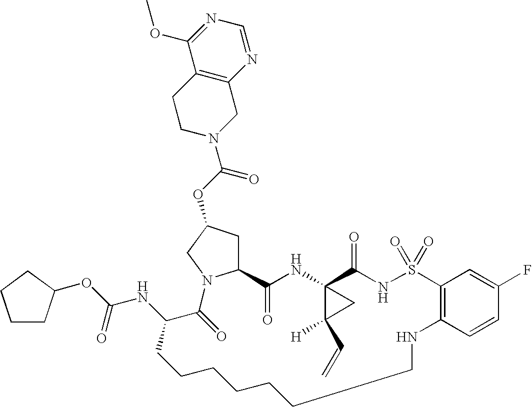 | Example 89: (1R,2S,2′R,6′S,24a′S)-6′- {[(cyclopentyloxy)carbonyl]amino}-17′-fluoro- 19′,19′-dioxido-5′,21′,24′-trioxo-2-vinyl- 1′,2′,3′,5′,6′,7′,8′,9′,10′,11′,12′,13′,14′,20′,21′,23′, 24′,24a′-octadecahydrospiro[cyclopropane- 1,22′-pyrrolo[2,1-g] [1,2,5,8,18]benzothiatetraazacycloicosin]-2′-yl 4-methoxy-5,8-dihydropyrido[3,4-d]pyrimidine- 7(6H)-carboxylate | M+ = 869.2 MS method D | | 6.41 HPLC method A |
| 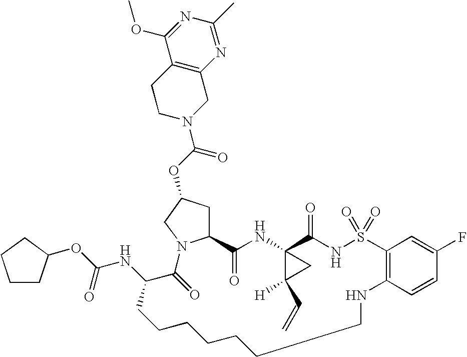 | Example 90: (1R,2S,2′R,6′S,24a′S)-6′- {[(cyclopentyloxy)carbonyl]amino}-17′-fluoro- 19′,19′-dioxido-5′,21′,24′-trioxo-2-vinyl- 1′,2′,3′,5′,6′,7′,8′,9′,10′,11′,12′,13′,14′,20′,21′,23′, 24′,24a′-octadecahydrospiro[cyclopropane- 1,22′-pyrrolo[2,1-g] [1,2,5,8,18]benzothiatetraazacycloicosin]-2′-yl 4-methoxy-2-methyl-5,8-dihydropyrido[3,4- d]pyrimidine-7(6H)-carboxylate | M+ = 883.2 MS method D | | 5.89 HPLC method A |
| 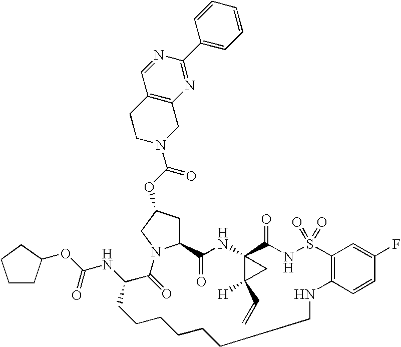 | Example 91: (1R,2S,2′R,6′S,24a′S)-6′- {[(cyclopentyloxy)carbonyl]amino}-17′-fluoro- 19′,19′-dioxido-5′,21′,24′-trioxo-2-vinyl- 1′,2′,3′,5′,6′,7′,8′,9′,10′,11′,12′,13′,14′,20′,21′,23′, 24′,24a′-octadecahydrospiro[cyclopropane- 1,22′-pyrrolo[2,1-g] [1,2,5,8,18]benzothiatetraazacycloicosin]-2′-yl 2-phenyl-5,8-dihydropyrido[3,4-d]pyrimidine- 7(6H)-carboxylate | M+ = 915.2 MS method D | | 7.16 HPLC method A |
| 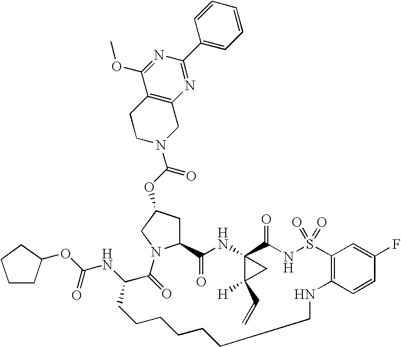 | Example 92: (1R,2S,2′R,6′S,24a′S)-6′- {[(cyclopentyloxy)carbonyl]amino}-17′-fluoro- 19′,19′-dioxido-5′,21′,24′-trioxo-2-vinyl- 1′,2′,3′,5′,6′,7′,8′,9′,10′,11′,12′,13′,14′,20′,21′,23′, 24′,24a′-octadecahydrospiro[cyclopropane- 1,22′-pyrrolo[2,1-g] [1,2,5,8,18]benzothiatetraazacycloicosin]-2′-yl 4-methoxy-2-phenyl-5,8-dihydropyrido[3,4- d]pyrimidine-7(6H)-carboxylate | M+ = 945.3 MS method D | | 7.39 HPLC method A |
| 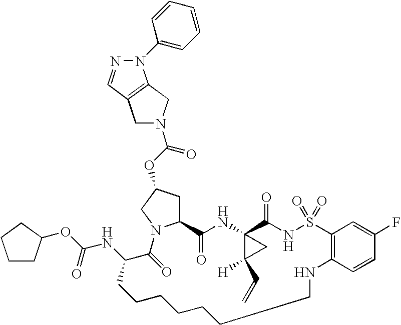 | Example 93: (1R,2S,2′R,6′S,24a′S)-6′- {[(cyclopentyloxy)carbonyl]amino}-17′-fluoro- 19′,19′-dioxido-5′,21′,24′-trioxo-2-vinyl-1′,2′,3′,5′, 6′,7′,8′,9′,10′,11′,12′,13′,14′,20′,21′,23′,24′,24a′- octadecahydrospiro[cyclopropane-1,22′-pyrrolo [2,1-g][1,2,5,8,18]benzothiatetraazacycloicosin]- 2′-yl 1-phenyl-4,6-dihydropyrrolo[3,4- c]pyrazole-5(1H)-carboxylate | M+ = 889.3 MS method D | | 6.912 HPLC method A |
| 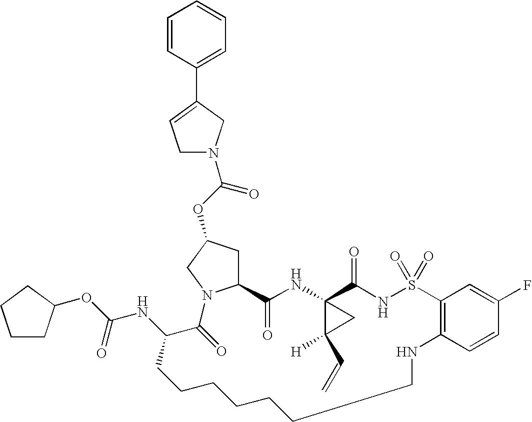 | Example 94: (1R,2S,2′R,6′S,24a′S)-6′- {[(cyclopentyloxy)carbonyl]amino}-17′-fluoro- 19′,19′-dioxido-5′,21′,24′-trioxo-2-vinyl-1′,2′,3′,5′, 6′,7′,8′,9′,10′,11′,12′,13′,14′,20′,21′,23′,24′,24a′- octadecahydrospiro[cyclopropane-1,22′-pyrrolo [2,1-g][1,2,5,8,18]benzothiatetraazacycloicosin]- 2′-yl 3-phenyl-2,5-dihydro-1H-pyrrole-1- carboxylate | M+ = 848.8 MS method D | | 7.027 HPLC method A |
| 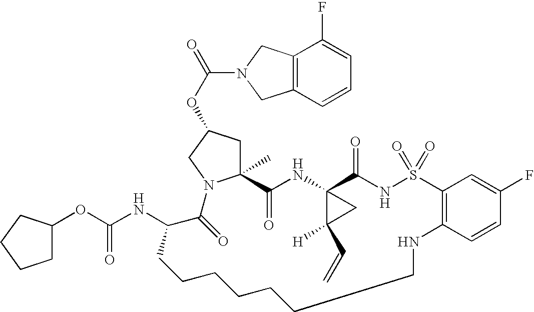 | Example 95: (1R,2S,2′R,6′S,24a′S)-6′- {[(cyclopentyloxy)carbonyl]amino}-17′-fluoro- 24a′-methyl-19′,19′-dioxido-5′,21′,24′-trioxo-2- vinyl-1′,2′,3′,5′,6′,7′,8′,9′,10′,11′,12′,13′,14′,20′,21′, 23′,24′,24a′-octadecahydrospiro[cyclopropane- 1,22′-pyrrolo[2,1-g][1,2,5,8,18]benzothiatetraaza- cycloicosin]-2′-yl 4-fluoro-1,3-dihydro-2H- isoindole-2-carboxylate | M+ = 854.7 MS method D | | 7.06 HPLC method A |
| 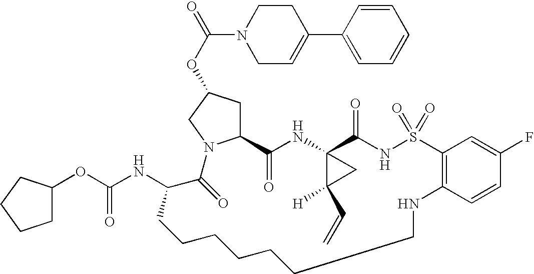 | Example 96: (1R,2S,2′R,6′S,24a′S)-6′- {[(cyclopentyloxy)carbonyl]amino}-17′-fluoro- 19′,19′-dioxido-5′,21′,24′-trioxo-2-vinyl-1′,2′,3′,5′, 6′,7′,8′,9′,10′,11′,12′,13′,14′,20′,21′,23′,24′,24a′- octadecahydrospiro[cyclopropane-1,22′-pyrrolo [2,1-g][1,2,5,8,18]benzothiatetraazacycloicosin]- 2′-yl 4-phenyl-3,6-dihydropyridine-1(2H)- carboxylate | M+ = 862.8 MS method D | | 7.18 HPLC method A |
| 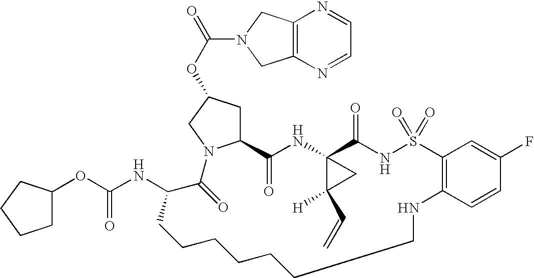 | Example 97: (1R,2S,2′R,6′S,24a′S)-6′- {[(cyclopentyloxy)carbonyl]amino}-17′-fluoro- 19′,19′-dioxido-5′,21′,24′-trioxo-2-vinyl-1′,2′,3′,5′, 6′,7′,8′,9′,10′,11′,12′,13′,14′,20′,21′,23′,24′,24a′- octadecahydrospiro[cyclopropane-1,22′-pyrrolo [2,1-g][1,2,5,8,18]benzothiatetraazacycloicosin]- 2′-yl 5,7-dihydro-6H-pyrrolo[3,4-b]pyrazine-6- carboxylate | M+ = 824.6 MS method D | | 6.26 HPLC method A |
| 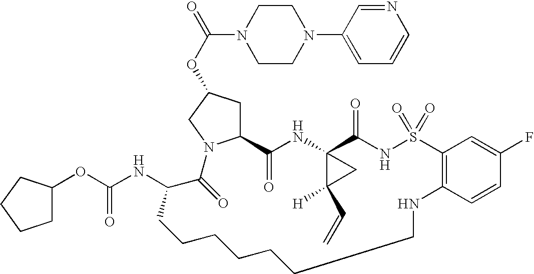 | Example 98: (1R,2S,2′R,6′S,24a′S)-6′- {[(cyclopentyloxy)carbonyl]amino}-17′-fluoro- 19′,19′-dioxido-5′,21′,24′-trioxo-2-vinyl-1′,2′,3′,5′, 6′,7′,8′,9′,10′,11′,12′,13′,14′,20′,21′,23′,24′,24a′- octadecahydrospiro[cyclopropane-1,22′-pyrrolo [2,1-g][1,2,5,8,18]benzothiatetraazacycloicosin]- 2′-yl 4-pyridin-3-ylpiperazine-1-carboxylate | | 3.485; M + H = 868.3 LC MS method E | 3.480 HPLC method C |
| 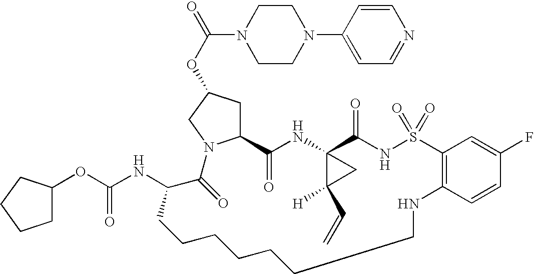 | Example 99: (1R,2S,2′R,6′S,24a′S)-6′- {[(cyclopentyloxy)carbonyl]amino}-17′-fluoro- 19′,19′-dioxido-5′,21′,24′-trioxo-2-vinyl-1′,2′,3′,5′, 6′,7′,8′,9′,10′,11′,12′,13′,14′,20′,21′,23′,24′,24a′- octadecahydrospiro[cyclopropane-1,22′-pyrrolo [2,1-g][1,2,5,8,18]benzothiatetraazacycloicosin]- 2′-yl 4-pyridin-4-ylpiperazine-1-carboxylate | | 3.398; M + H = 868.3 LC MS method E | 3.673 HPLC method C |
| 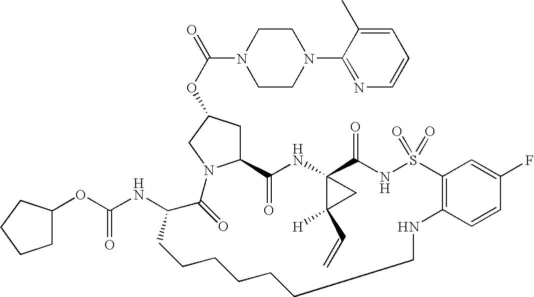 | Example 100: (1R,2S,2′R,6′S,24a′S)-6′- {[(cyclopentyloxy)carbonyl]amino}-17′-fluoro- 19′,19′-dioxido-5′,21′,24′-trioxo-2-vinyl-1′,2′,3′,5′, 6′,7′,8′,9′,10′,11′,12′,13′,14′,20′,21′,23′,24′,24a′- octadecahydrospiro[cyclopropane-1,22′-pyrrolo [2,1-g][1,2,5,8,18]benzothiatetraazacycloicosin]- 2′-yl 4-(3-methylpyridin-2-yl)piperazine-1- carboxylate | | 4.189; M + H = 882.3 LC MS method E | 3.771 HPLC method C |
| 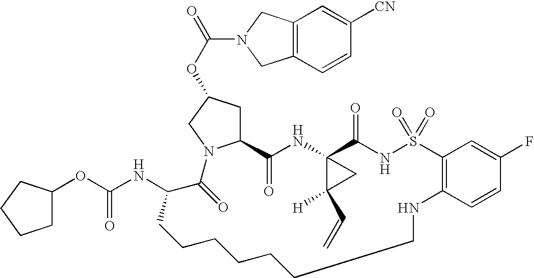 | Example 101: (1R,2S,2′R,6′S,24a′S)-6′- {[(cyclopentyloxy)carbonyl]amino}-17′-fluoro- 19′,19′-dioxido-5′,21′,24′-trioxo-2-vinyl-1′,2′,3′,5′, 6′,7′,8′,9′,10′,11′,12′,13′,14′,20′,21′,23′,24′,24a′- octadecahydrospiro[cyclopropane-1,22′-pyrrolo [2,1-g][1,2,5,8,18]benzothiatetraazacycloicosin]- 2′-yl 5-cyano-1,3-dihydro-2H-isoindole-2- carboxylate | | 4.387; M − H = 846.3 LC MS method E | 4.524 HPLC method C |
| 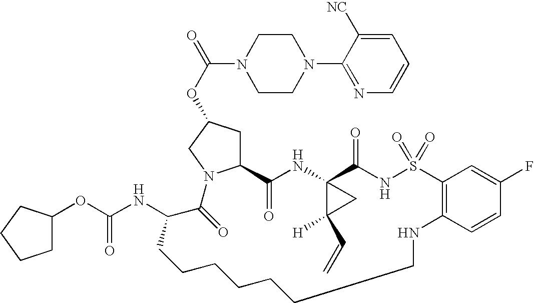 | Example 102: (1R,2S,2′R,6′S,24a′S)-6′- {[(cyclopentyloxy)carbonyl]amino}-17′-fluoro- 19′,19′-dioxido-5′,21′,24′-trioxo-2-vinyl-1′,2′,3′,5′, 6′,7′,8′,9′,10′,11′,12′,13′,14′,20′,21′,23′,24′,24a′- octadecahydrospiro[cyclopropane-1,22′-pyrrolo [2,1-g][1,2,5,8,18]benzothiatetraazacycloicosin]- 2′-yl 4-(3-cyanopyridin-2-yl)piperazine-1- carboxylate | | 4.456; M + H = 893.2 LC MS method E | 4.583 HPLC method C |
| 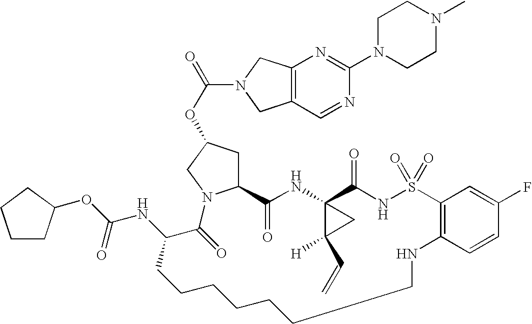 | Example 103: 2-(4-Methyl-piperazin-1-yl)-5,7- dihydro-pyrrolo[3,4-d]pyrimidine-6-carboxylic acid (8S,10R,14S)-14-cyclopentyloxycarbonyl- amino-5-[(1R,2S)-1-carbonylamino-2-vinyl- cyclopropyl]-26-fluoro-2,2,4,7,13-pentaoxo- 2Λ*6*-thia-3,6,12,22-tetraaza- tricyclo[21.4.0.0*8,12*]heptacosa-1(23),24,26- trien-10-yl ester | M + 1 = 923 MS method D | | 5.27 HPLC method A |
| 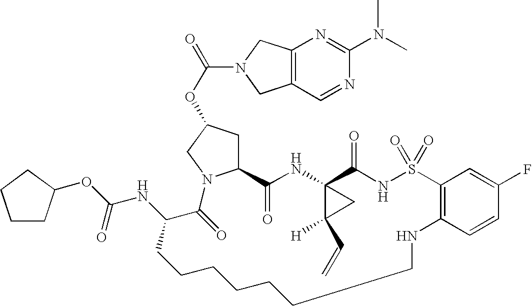 | Example 104: 2-Dimethylamino-5,7-dihydro- pyrrolo[3,4-d]pyrimidine-6-carboxylic acid (8S,10R,14S)-14-cyclopentyloxycarbonyl- amino-5-[(1R,2S)-1-carbonylamino-2-vinyl- cyclopropyl]-26-fluoro-2,2,4,7,13-pentaoxo- 2Λ*6*-thia-3,6,12,22-tetraaza- tricyclo[21.4.0.0*8,12*]heptacosa-1(23),24,26- trien-10-yl ester | M + 1 = 868 MS method D | | 5.93 HPLC method A |
| 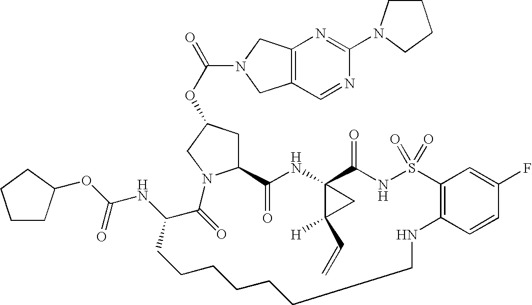 | Example 105: 2-Pyrrolidin-1-yl-5,7-dihydro- pyrrolo[3,4-d]pyrimidine-6-carboxylic acid (8S,10R,14S)-14-cyclopentyloxycarbonyl- amino-5-[(1R,2S)-1-carbonylamino-2-vinyl- cyclopropyl]-26-fluoro-2,2,4,7,13-pentaoxo- 2Λ*6*-thia-3,6,12,22-tetraaza- tricyc1o[21.4.0.0*8,12*]heptacosa-1(23),24,26- trien-10-yl ester | M + 1 = 894 MS method D | | 5.82 HPLC method A |
| 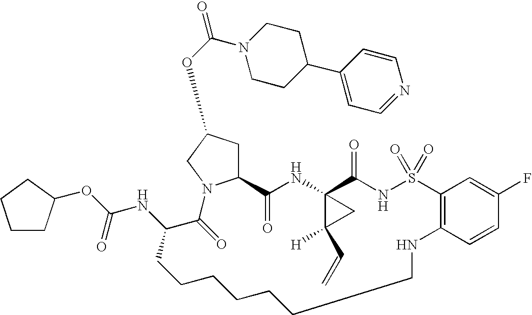 | Example 106: 3,4,5,6-Tetrahydro-2H- [4,4′]bipyridinyl-1-carboxylic acid (8S,10R,14S)- 14-cyclopentyloxycarbonyl-amino-5-[(1R,2S)-1- carbonylamino-2-vinyl-cyclopropyl]-26-fluoro- 2,2,4,7,13-pentaoxo-2Λ*6*-thia-3,6,12,22- tetraaza-tricyclo[21.4.0.0*8,12*]heptacosa- 1(23),24,26-trien-10-yl ester | M − 1 = 865 MS method D | | 5.50 HPLC method A |
| 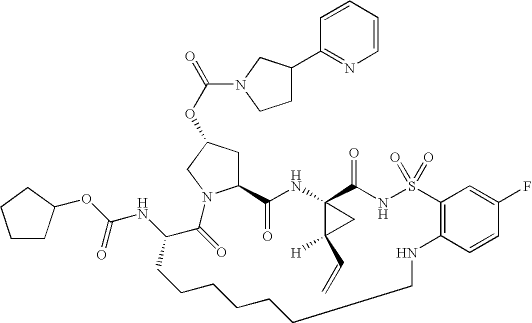 | Example 107: 3-Pyridin-2-yl-pyrrolidine-1- carboxylic acid (8S,10R,14S)-14- cyclopentyloxycarbonyl-amino-5-[(1R,2S)-1- carbonylamino-2-vinyl-cyclopropyl]-26-fluoro- 2,2,4,7,13-pentaoxo-2Λ*6*-thia-3,6,12,22- tetraaza-tricyclo[21.4.0.0*8,12*]heptacosa- 1(23),24,26-trien-10-yl ester | M − 1 = 851 MS method D | | 5.42 HPLC method A |
| 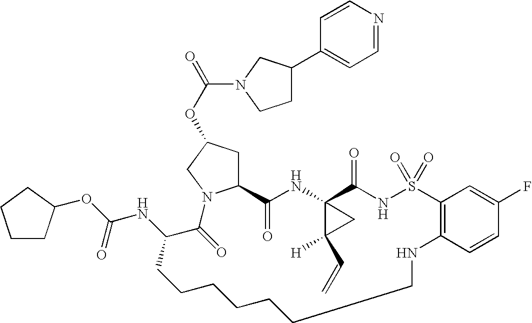 | Example 108: 3-Pyridin-4-yl-pyrrolidine-1- carboxylic acid (8S,10R,14S)-14- cyclopentyloxycarbonyl-amino-5-[(1R,2S)-1- carbonylamino-2-vinyl-cyclopropyl]-26-fluoro- 2,2,4,7,13-pentaoxo-2Λ*6*-thia-3,6,12,22- tetraaza-tricyclo[21.4.0.0*8,12*]heptacosa- 1(23),24,26-trien-10-yl ester | M − 1 = 851 MS method D | | 5.36 HPLC method A |
| 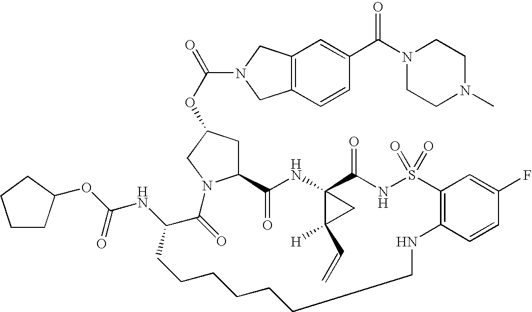 | Example 109: 5-(4-Methyl-piperazine-1- carbonyl)-1,3-dihydro-isoindole-2-carboxylic acid (8S,10R,14S)-14-cyclopentyloxycarbonyl- amino-5-[(1R,2S)-1-carbonylamino-2-vinyl- cyclopropyl]-26-fluoro-2,2,4,7,13-pentaoxo- 2Λ*6*-thia-3,6,12,22-tetraaza- tricyclo[21.4.0.0*8,12*]heptacosa-1(23),24,26- trien-10-yl ester | M − 1 = 948 MS method D | | 5.35 HPLC method A |
| 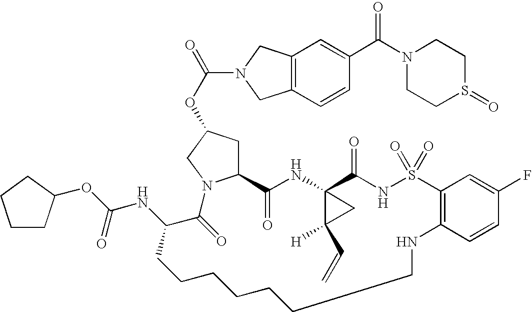 | Example 110: 5-(1-Oxo-1Λ*4*-thiomorpholine- 4-carbonyl)-1,3-dihydro-isoindole-2-carboxylic acid (8S,10R,14S)-14-cyclopentyloxycarbonyl- amino-5-[(1R,2S)-1-carbonylamino-2-vinyl- cyclopropyl]-26-fluoro-2,2,4,7,13-pentaoxo- 2Λ*6*-thia-3,6,12,22-tetraaza- tricyclo[21.4.0.0*8,12*] heptacosa-1(23),24,26-trien-10-yl ester | M − 1 = 967 MS method D | | 5.85 HPLC method A |
| 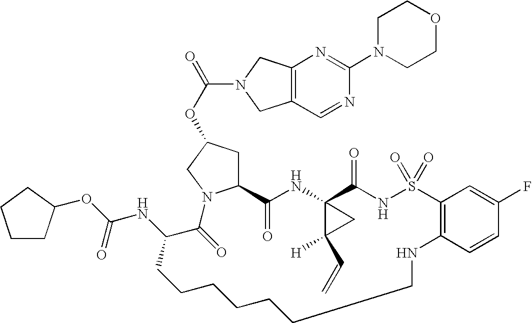 | Example 111: 2-Morpholin-4-yl-5,7-dihydro- pyrrolo[3,4-d]pyrimidine-6-carboxylic acid (8S,10R,14S)-14-cyclopentyloxycarbonyl- amino-5-[(1R,2S)-1-carbonylamino-2-vinyl- cyclopropyl]-26-fluoro-2,2,4,7,13-pentaoxo- 2Λ*6*-thia-3,6,12,22-tetraaza- tricyclo[21.4.0.0*8,12*] heptacosa-1(23),24,26-trien-10-yl ester | M + 1 = 910 MS method D | | 6.46 HPLC method A |
| 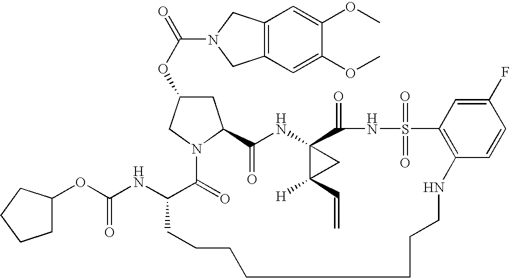 | Example 112: 5,6-Dimethoxy-1,3-dihydro- isoindole-2-carboxylic acid (8S,10R,14S)-14- cyclopentyloxycarbonyl-amino-5-[(1R,2S)-1- carbonylamino-2-vinyl-cyclopropyl]-26-fluoro- 2,2,4,7,13-pentaoxo-2Λ*6*-thia-3,6,12,22- tetraaza-tricyclo[21.4.0.0*8,12*]heptacosa- 1(23),24,26-trien-10-yl ester | M + 1 = 884 MS method D | | 6.62 HPLC method A |
| 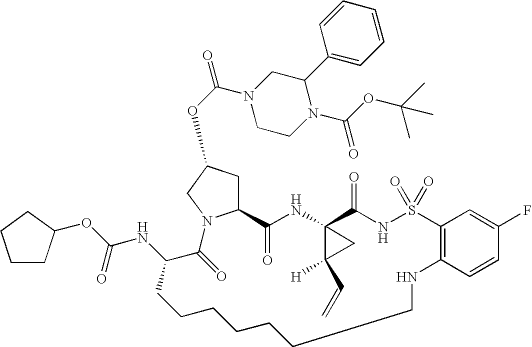 | Example 113: 2-Phenyl-piperazine-1,4- dicarboxylic acid 1-tert-butyl ester (8S,10R,14S)-14-cyclopentyloxycarbonyl- amino-5-[(1R,2S)-1-carbonylamino-2-vinyl- cyclopropyl]-26-fluoro-2,2,4,7,13-pentaoxo- 2Λ*6*-thia-3,6,12,22-tetraaza- tricyclo[21.4.0.0*8,12*] heptacosa-1(23),24,26-trien-10-yl ester | M + 1 = 967 MS method D | | 7.35 HPLC method A |
| 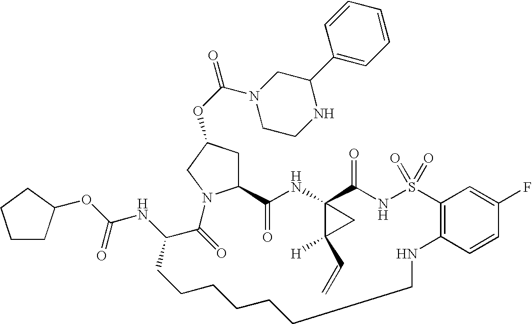 | Example 114: 3-Phenyl-piperazine-1-carboxylic acid (8S,10R,14S)-14-cyclopentyloxycarbonyl- amino-5-[(1R,2S)-1-carbonylamino-2-vinyl- cyclopropyl]-26-fluoro-2,2,4,7,13-pentaoxo- 2Λ*6*-thia-3,6,12,22-tetraaza- tricyclo[21.4.0.0*8,12*] heptacosa-1(23),24,26-trien-10-yl ester | M + 1 = 967 MS method D | | 5.62 HPLC method A |
| 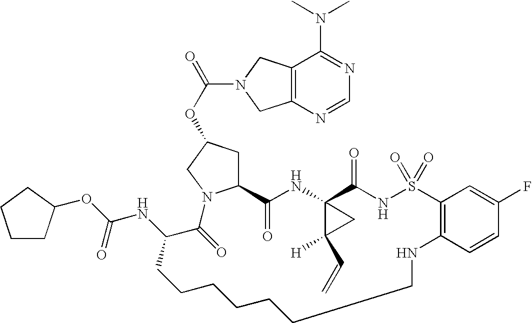 | Example 115: 4-Dimethylamino-5,7-dihydro- pyrrolo[3,4-d]pyrimidine-6-carboxylic acid (8S,10R,14S)-14-cyclopentyloxycarbonyl- amino-5-[(1R,2S)-1-carbonylamino-2-vinyl- cyclopropyl]-26-fluoro-2,2,4,7,13-pentaoxo- 2Λ*6*-thia-3,6,12,22-tetraaza- tricyclo[21.4.0.0*8,12*] heptacosa-1(23),24,26-trien-10-yl ester | M + 1 = 868 MS method D | | 5.44 HPLC method A |
| 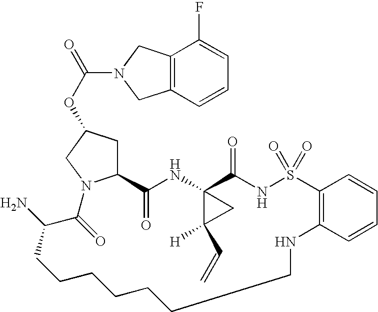 | Example 116: (1R,2S,2′R,6′S,24a′S)-6′-amino- 19′,19′-dioxido-5′,21′,24′-trioxo-2-vinyl- 1′,2′,3′,5′,6′,7′,8′,9′,10′,11′,12′,13′,14′,20′,21′,23′, 24′,24a′-octadecahydrospiro[cyclopropane- 1,22′-pyrrolo[2,1-g] [1,2,5,8,18]benzothiatetraazacycloicosin]-2′-yl 4-fluoro-1,3-dihydro-2H-isoindole-2-carboxylate | M + H = 711.2 MS method D | | 4.51 HPLC method A |
| 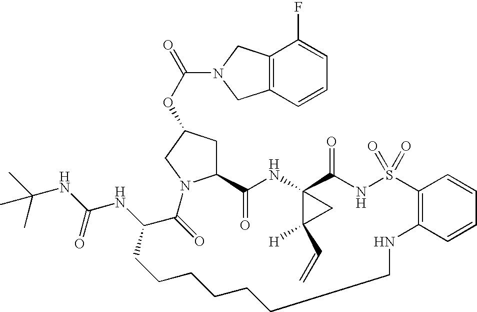 | Example 117: (1R,2S,2′R,6′S,24a′S)-6′-[(tert- butylcarbamoyl)amino]-19′,19′-dioxido- 5′,21′,24′-trioxo-2-vinyl- 1′,2′,3′,5′,6′,7′,8′,9′,10′,11′,12′,13′,14′,20′,21′,23′, 24′,24a′-octadecahydrospiro[cyclopropane- 1,22′-pyrrolo[2,1-g] [1,2,5,8,18]benzothiatetraazacycloicosin]-2′-yl 4-fluoro-1,3-dihydro-2H-isoindole-2-carboxylate | M + H = 810.3 MS method D | | 5.51 HPLC method A |
| 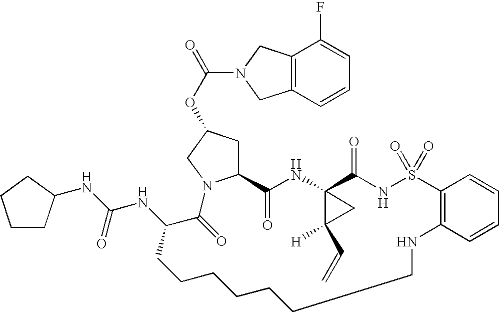 | Example 118: (1R,2S,2′R,6′S,24a′S)-6′- [(cyclopentylcarbamoyl)amino]-19′,19′-dioxido- 5′,21′,24′-trioxo-2-vinyl-1′,2′,3′,5′,6′,7′,8′,9′,10′, 11′,12′,13′,14′,20′,21′,23′,24′,24a′-octadecahydro- spiro[cyclopropane-1,22′-pyrrolo[2,1- g][1,2,5,8,18]benzothiatetraazacycloicosin]-2′-yl 4-fluoro-1,3-dihydro-2H-isoindole-2-carboxylate | M + H = 822.3 MS method D | | 5.49 HPLC method A |
| 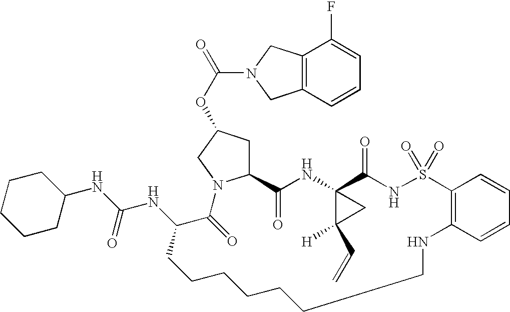 | Example 119: (1R,2S,2′R,6′S,24a′S)-6′- [(cyclohexylcarbamoyl)amino]-19′,19′-dioxido- 5′,21′,24′-trioxo-2-vinyl-1′,2′,3′,5′,6′,7′,8′,9′,10′, 11′,12′,13′,14′,20′,21′,23′,24′,24a′-octadecahydro- spiro[cyclopropane-1,22′-pyrrolo[2,1- g][1,2,5,8,18]benzothiatetraazacycloicosin]-2′-yl 4-fluoro-1,3-dihydro-2H-isoindole-2-carboxylate | M+ = 836.2 LC MS method D | | 5.66 HPLC method A |
| 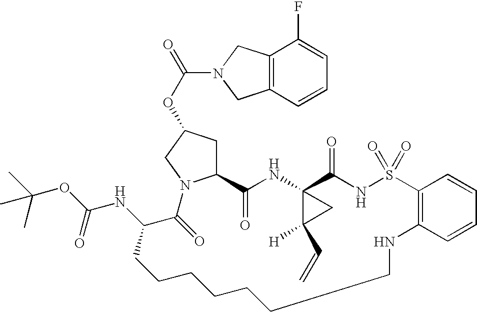 | Example 120: (1R,2S,2′R,6′S,24a′S)-6′-[(tert- butoxycarbonyl)amino]-19′,19′-dioxido- 5′,21′,24′-trioxo-2-vinyl-1′,2′,3′,5′,6′,7′,8′,9′,10′, 11′,12′,13′,14′,20′,21′,23′,24′,24a′-octadecahydro- spiro[cyclopropane-1,22′-pyrrolo[2,1- g][1,2,5,8,18]benzothiatetraazacycloicosin]-2′-yl 4-fluoro-1,3-dihydro-2H-isoindole-2-carboxylate | M + H = 811.2 MS method D | | 5.87 HPLC method A |
| 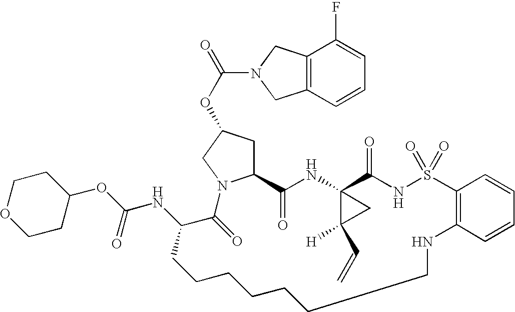 | Example 121: (1R,2S,2′R,6′S,24a′S)-19′,19′- dioxido-5′,21′,24′-trioxo-6′-{[(tetrahydro-2H- pyran-4-yloxy)carbonyl]amino}-2-vinyl- 1′,2′,3′,5′,6′,7′,8′,9′,10′,11′,12′,13′,14′,20′,21′,23′, 24′,24a′-octadecahydrospiro[cyclopropane- 1,22′-pyrrolo[2,1-g][1,2,5,8,18] benzothiatetraazacycloicosin]-2′-yl 4-fluoro-1,3-dihydro-2H-isoindole-2-carboxylate | M + H = 839.3 MS method D | | 5.38 HPLC method A |
| 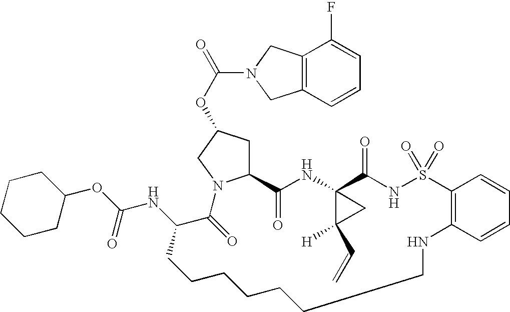 | Example 122: (1R,2S,2′R,6′S,24a′S)-6′- {[(cyclohexyloxy)carbonyl]amino}-19′,19′- dioxido-5′,21′,24′-trioxo-2-vinyl-1′,2′,3′,5′,6′,7′, 8′,9′,10′,11′,12′,13′,14′,20′,21′,23′,24′,24a′- octadecahydrospiro[cyclopropane-1,22′-pyrrolo [2,1-g][1,2,5,8,18]benzothiatetraazacycloicosin]- 2′-yl 4-fluoro-1,3-dihydro-2H-isoindole-2- carboxylate | M+ = 837.2 MS method D | | 6.10 HPLC method A |
| 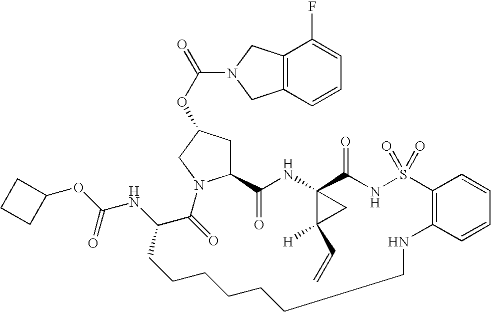 | Example 123: (1R,2S,2′R,6′S,24a′S)-6′- {[(cyclobutyloxy)carbonyl]amino}-19′,19′- dioxido-5′,21′,24′-trioxo-2-vinyl-1′,2′,3′,5′,6′,7′, 8′,9′,10′,11′,12′,13′,14′,20′,21′,23′,24′,24a′- octadecahydrospiro[cyclopropane-1,22′-pyrrolo [2,1-g][1,2,5,8,18]benzothiatetraazacycloicosin]- 2′-yl 4-fluoro-1,3-dihydro-2H-isoindole-2- carboxylate | M+ = 809.2 MS method D | | 5.79 HPLC method A |
| 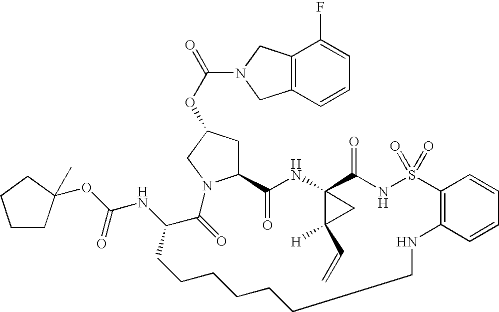 | Example 124: (1R,2S,2′R,6′S,24a′S)-6′-({[(1- methylcyclopentyl)oxy]carbonyl}amino)-19′,19′- dioxido-5′,21′,24′-trioxo-2-vinyl-1′,2′,3′,5′,6′,7′, 8′,9′,10′,11′,12′,13′,14′,20′,21′,23′,24′,24a′- octadecahydrospiro[cyclopropane-1,22′-pyrrolo [2,1-g][1,2,5,8,18]benzothiatetraazacycloicosin]- 2′-yl 4-fluoro-1,3-dihydro-2H-isoindole-2- carboxylate | M+ = 837.2 MS method D | | 6.16 HPLC method A |
| 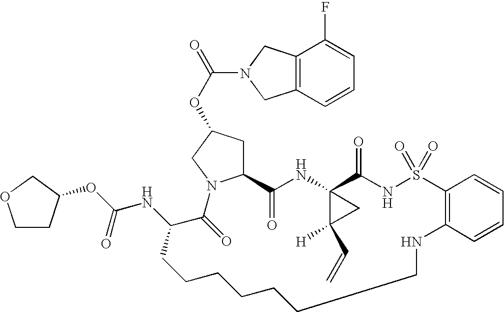 | Example 125: (1R,2S,2′R,6′S,24a′S)-19′,19′- dioxido-5′,21′,24′-trioxo-6′-({[(3R)-tetrahydro- furan-3-yloxy]carbonyl}amino)-2-vinyl-1′,2′,3′, 5′,6′,7′,8′,9′,10′,11′,12′,13′,14′,20′,21′,23′,24′,24a′- octadecahydrospiro[cyclopropane-1,22′-pyrrolo [2,1-g][1,2,5,8,18]benzothiatetraazacycloicosin]- 2′-yl 4-fluoro-1,3-dihydro-2H-isoindole-2- carboxylate | M+ = 825.3 MS method D | | 5.26 HPLC method A |
| 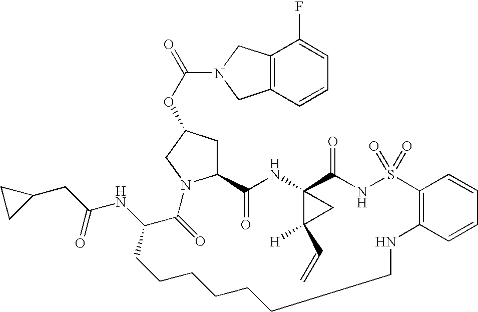 | Example 126: (1R,2S,2′R,6′S,24a′S)-6′- [(cyclopropylacetyl)amino]-19′,19′-dioxido- 5′,21′,24′-trioxo-2-vinyl-1′,2′,3′,5′,6′,7′,8′,9′,10′, 11′,12′,13′,14′,20′,21′,23′,24′,24a′-octadecahydro- spiro[cyclopropane-1,22′-pyrrolo[2,1- g][1,2,5,8,18]benzothiatetraazacycloicosin]-2′-yl 4-fluoro-1,3-dihydro-2H-isoindole-2-carboxylate | M+ = 793.2 MS method D | | 5.32 HPLC method A |
| 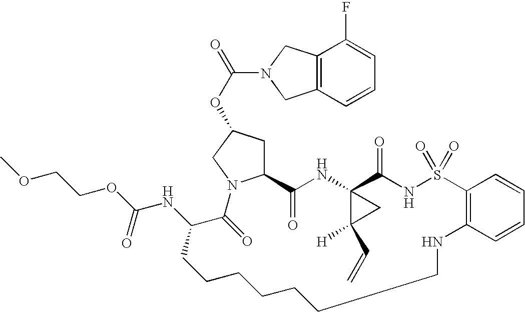 | Example 127: (1R,2S,2′R,6′S,24a′S)-6′-{[(2- methoxyethoxy)carbonyl]amino}-19′,19′- dioxido-5′,21′,24′-trioxo-2-vinyl- 1′,2′,3′,5′,6′,7′,8′,9′,10′,11′,12′,13′,14′,20′,21′,23′, 24′,24a′-octadecahydrospiro[cyclopropane- 1,22′-pyrrolo[2,1-g] [1,2,5,8,18]benzothiatetraazacycloicosin]-2′-yl 4-fluoro-1,3-dihydro-2H-isoindole-2-carboxylate | M+ = 813.3 MS method D | | 5.24 HPLC method A |
| 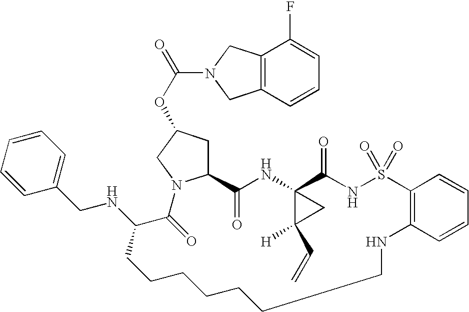 | Example 128: (1R,2S,2′R,6′S,24a′S)-6′- (benzylamino)-19′,19′-dioxido-5′,21′,24′-trioxo- 2-vinyl-1′,2′,3′,5′,6′,7′,8′,9′,10′,11′,12′,13′,14′,20′, 21′,23′,24′,24a′-octadecahydrospiro [cyclopropane-1,22′-pyrrolo[2,1-g] [1,2,5,8,18]benzothiatetraazacycloicosin]-2′-yl 4- fluoro-1,3-dihydro-2H-isoindole-2-carboxylate | M+ = 801.2 MS method D | | 4.88 HPLC method A |
| 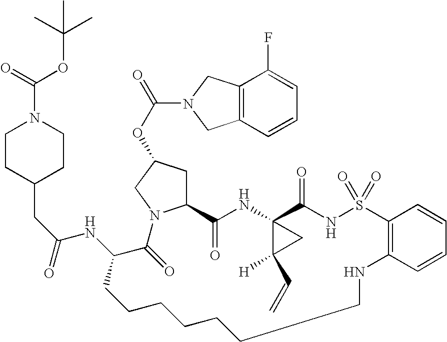 | Example 129: (1R,2S,2′R,6′S,24a′S)-6′-({[1-(tert- butoxycarbonyl)piperidin-4-yl]acetyl}amino)- 19′,19′-dioxido-5′,21′,24′-trioxo-2-vinyl-1′,2′,3′,5′, 6′,7′,8′,9′,10′,11′,12′,13′,14′,20′,21′,23′,24′,24a′- octadecahydrospiro[cyclopropane-1,22′-pyrrolo [2,1-g][1,2,5,8,18]benzothiatetraazacycloicosin]- 2′-yl 4-fluoro-1,3-dihydro-2H-isoindole-2- carboxylate | M+ = 936.5 MS method D | | 5.72 HPLC method A |
| 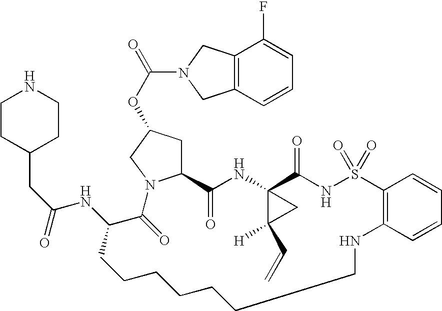 | Example 130: (1R,2S,2′R,6′S,24a′S)-19′,19′- dioxido-5′,21′,24′-trioxo-6′-[(piperidin-4-yl acetyl)amino]-2-vinyl-1′,2′,3′,5′,6′,7′,8′,9′,10′,11′, 12′,13′,14′,20′,21′,23′,24′,24a′-octadecahydro- spiro[cyclopropane-1,22′-pyrrolo[2,1- g][1,2,5,8,18]benzothiatetraazacycloicosin]-2′-yl 4-fluoro-1,3-dihydro-2H-isoindole-2-carboxylate | M+ = 936.2 MS method D | | 4.54 HPLC method A |
| 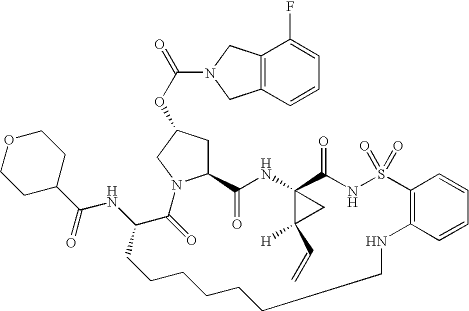 | Example 131: (1R,2S,2′R,6′S,24a′S)-19′,19′- dioxido-5′,21′,24′-trioxo-6′-[(tetrahydro-2H- pyran-4-ylcarbonyl)amino]-2-vinyl-1′,2′,3′,5′, 6′,7′,8′,9′,10′,11′,12′,13′,14′,20′,21′,23′,24′,24a′- octadecahydrospiro[cyclopropane-1,22′-pyrrolo [2,1-g][1,2,5,8,18]benzothiatetraazacycloicosin]- 2′-yl 4-fluoro-1,3-dihydro-2H-isoindole-2- carboxylate | M+ = 823.3 MS method D | | 5.13 HPLC method A |
| 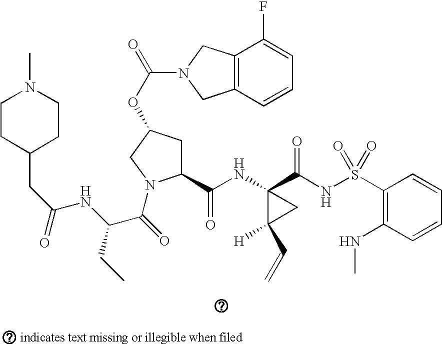 | Example 132: (1R,2S,2′R,6′S,24a′S)-6′-{[(1- methylpiperidin-4-yl)acetyl]amino}-19′,19′- dioxido-5′,21′,24′-trioxo-2-vinyl-1′,2′,3′,5′,6′,7′, 8′,9′,10′,11′,12′,13′,14′,20′,21′,23′,24′,24a′- octadecahydrospiro[cyclopropane-1,22′-pyrrolo [2,1-g][1,2,5,8,18]benzothiatetraazacycloicosin]- 2′-yl 4-fluoro-1,3-dihydro-2H-isoindole-2- carboxylate | M+ = 850.5 MS method D | | 4.64 HPLC method A |
| 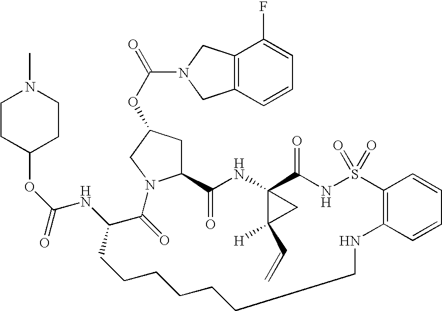 | Example 133: (1R,2S,2′R,6′S,24a′S)-6′-({[(1- methylpiperidin-4-yl)oxy]carbonyl}amino)- 19′,19′-dioxido-5′,21′,24′-trioxo-2-vinyl-1′,2′,3′,5′, 6′,7′,8′,9′,10′,11′,12′,13′,14′,20′,21′,23′,24′,24a′- octadecahydrospiro[cyclopropane-1,22′-pyrrolo [2,1-g][1,2,5,8,18]benzothiatetraazacycloicosin]- 2′-yl 4-fluoro-1,3-dihydro-2H-isoindole-2- carboxylate | M+ = 852.2 MS method D | | 4.68 HPLC method A |
| 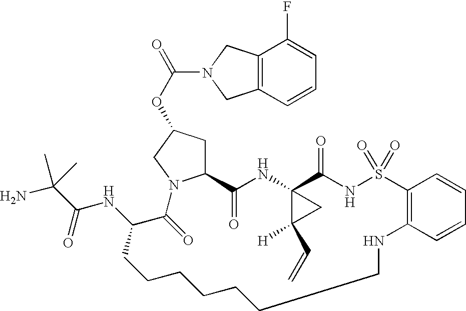 | Example 134: (1R,2S,2′R,6′S,24a′S)-6′-[(2- methylalanyl)amino]-19′,19′-dioxido-5′,21′,24′- trioxo-2-vinyl-1′,2′,3′,5′,6′,7′,8′,9′,10′,11′,12′, 13′,14′,20′,21′,23′,24′,24a′-octadeca- hydrospiro[cyclopropane-1,22′-pyrrolo[2,1- g][1,2,5,8,18]benzothiatetraazacycloicosin]-2′-yl 4-fluoro-1,3-dihydro-2H-isoindole-2-carboxylate | M+ = 796.3 MS method D | | 4.55 HPLC method A |
| 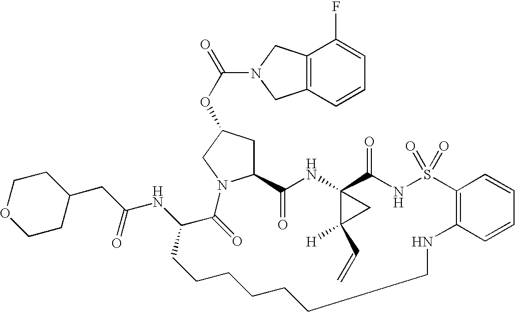 | Example 135: (1R,2S,2′R,6′S,24a′S)-19′,19′- dioxido-5′,21′,24′-trioxo-6′-[(tetrahydro-2H- pyran-4-ylacetyl)amino]-2-vinyl-1′,2′,3′,5′, 6′,7′,8′,9′,10′,11′,12′,13′,14′,20′,21′,23′,24′,24a′- octadecahydrospiro[cyclopropane-1,22′-pyrrolo [2,1-g][1,2,5,8,18]benzothiatetraazacycloicosin]- 2′-yl 4-fluoro-1,3-dihydro-2H-isoindole-2- carboxylate | M+ = 837.2 MS method D | | 5.19 HPLC method A |
| 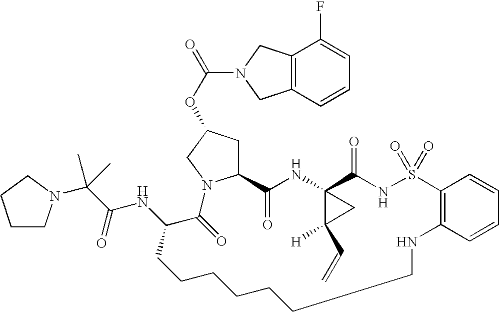 | Example 136: (1R,2S,2′R,6′S,24a′S)-6′-[(2- methyl-2-pyrrolidin-1-ylpropanoyl)amino]- 19′,19′-dioxido-5′,21′,24′-trioxo-2-vinyl-1′,2′,3′,5′, 6′,7′,8′,9′,10′,11′,12′,13′,14′,20′,21′,23′,24′,24a′- octadecahydrospiro[cyclopropane-1,22′-pyrrolo [2,1-g][1,2,5,8,18]benzothiatetraazacycloicosin]- 2′-yl 4-fluoro-1,3-dihydro-2H-isoindole-2- carboxylate | M+ = 850.5 MS method D | | 4.77 HPLC method A |
| 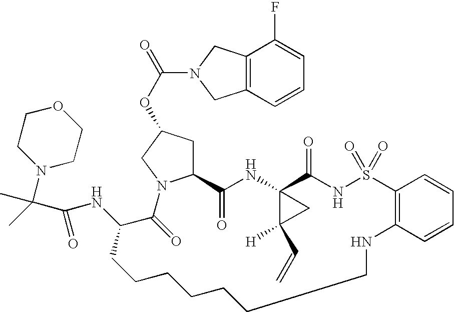 | Example 137: (1R,2S,2′R,6′S,24a′S)-6′-[(2- methyl-2-morpholin-4-ylpropanoyl)amino]- 19′,19′-dioxido-5′,21′,24′-trioxo-2-vinyl-1′,2′,3′,5′, 6′,7′,8′,9′,10′,11′,12′,13′,14′,20′,21′,23′,24′,24a′- octadecahydrospiro[cyclopropane-1,22′-pyrrolo [2,1-g][1,2,5,8,18]benzothiatetraazacycloicosin]- 2′-yl 4-fluoro-1,3-dihydro-2H-isoindole-2- carboxylate | M+ = 866.3 MS method D | | 4.70 HPLC method A |
| 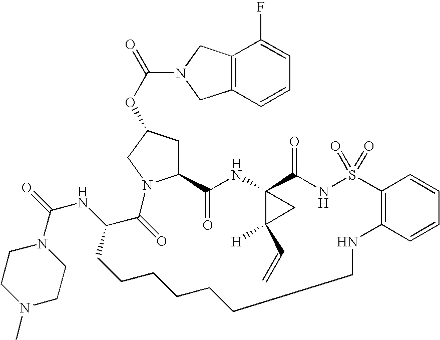 | Example 138: (1R,2S,2′R,6′S,24a′S)-6′-{[(4- methylpiperazin-1-yl)carbonyl]amino}-19′,19′- dioxido-5′,21′,24′-trioxo-2-vinyl-1′,2′,3′,5′, 6′,7′,8′,9′,10′,11′,12′,13′,14′,20′,21′,23′,24′,24a′- octadecahydrospiro[cyclopropane-1,22′-pyrrolo [2,1-g][1,2,5,8,18]benzothiatetraazacycloicosin]- 2′-yl 4-fluoro-1,3-dihydro-2H-isoindole-2- carboxylate | M+ = 837.5 MS method D | | 4.57 HPLC method A |
| 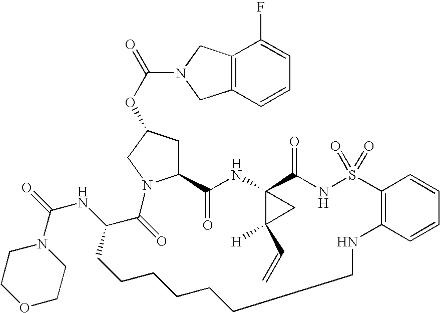 | Example 139: (1R,2S,2′R,6′S,24a′S)-6′- [(morpholin-4-ylcarbonyl)amino]-19′,19′- dioxido-5′,21′,24′-trioxo-2-vinyl-1′,2′,3′,5′,6′,7′,8′, 9′,10′,11′,12′,13′,14′,20′,21′,23′,24′,24a′-octadeca- hydrospiro[cyclopropane-1,22′-pyrrolo[2,1- g][1,2,5,8,18]benzothiatetraazacycloicosin]-2′-yl 4-fluoro-1,3-dihydro-2H-isoindole-2-carboxylate | M+ = 824.2 MS method D | | 5.15 HPLC method A |
| 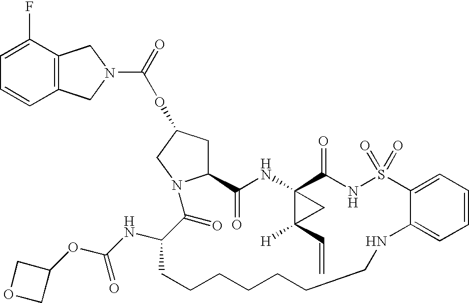 | Example 140: (1R,2S,2′R,6′S,24a′S)-6′- {[(oxetan-3-yloxy)carbonyl]amino}-19′,19′- dioxido-5′,21′,24′-trioxo-2-vinyl-1′,2′,3′,5′,6′,7′, 8′,9′,10′,11′,12′,13′,14′,20′,21′,23′,24′,24a′- octadecahydrospiro[cyclopropane-1,22′-pyrrolo [2,1-g][1,2,5,8,18]benzothiatetraazacycloicosin]- 2′-yl 4-fluoro-1,3-dihydro-2H-isoindole-2- carboxylate | M+ = 811.2 MS method D | | 5.16 HPLC method A |
| 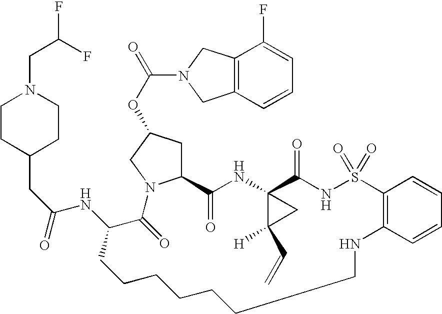 | Example 141: (1R,2S,2′R,6′S,24a′S)-6′-({[1-(2,2- difluoroethyl)piperidin-4-yl]acetyl}amino)- 19′,19′-dioxido-5′,21′,24′-trioxo-2-vinyl-1′,2′,3′,5′, 6′,7′,8′,9′,10′,11′,12′,13′,14′,20′,21′,23′,24′,24a′- octadecahydrospiro[cyclopropane-1,22′-pyrrolo [2,1-g][1,2,5,8,18]benzothiatetraazacycloicosin]- 2′-yl 4-fluoro-1,3-dihydro-2H-isoindole-2- carboxylate | M+ = 900.3 MS method D | | 4.67 HPLC method A |
| 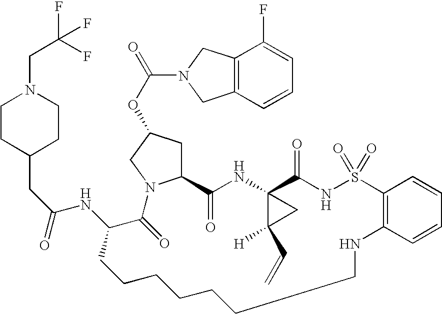 | Example 142: (1R,2S,2′R,6′S,24a′S)-19′,19′- dioxido-5′,21′,24′-trioxo-6′-({[1-(2,2,2- trifluoroethyl)piperidin-4-yl]acetyl}amino)-2- vinyl-1′,2′,3′,5′,6′,7′,8′,9′,10′,11′,12′,13′,14′,20′, 21′,23′,24′,24a′-octadecahydrospiro [cyclopropane-1,22′-pyrrolo[2,1- g][1,2,5,8,18]benzothiatetraazacycloicosin]-2′-yl 4-fluoro-1,3-dihydro-2H-isoindole-2-carboxylate | M+ = 918.3 MS method D | | 4.75 HPLC method A |
| 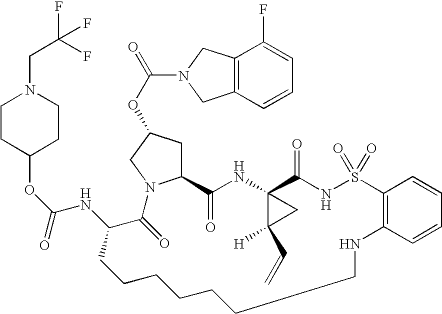 | Example 143: (1R,2S,2′R,6′S,24a′S)-19′,19′- dioxido-5′,21′,24′-trioxo-6′-[({[1-(2,2,2- trifluoroethyl)piperidin-4-yl]oxy}carbonyl) amino]-2-vinyl-1′,2′,3′,5′,6′,7′,8′,9′,10′,11′,12′, 13′,14′,20′,21′,23′,24′,24a′-octadecahydrospiro [cyclopropane-1,22′-pyrrolo[2,1- g][1,2,5,8,18]benzothiatetraazacycloicosin]-2′-yl 4-fluoro-1,3-dihydro-2H-isoindole-2-carboxylate | M+ = 920.2 MS method D | | 5.02 HPLC method A |
| 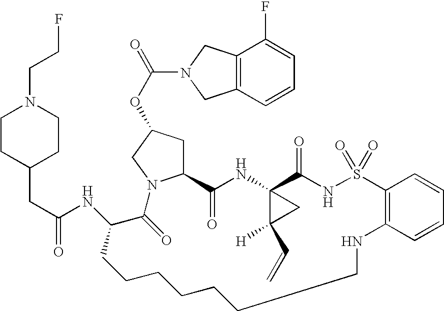 | Example 144: (1R,2S,2′R,6′S,24a′S)-6′-({[1-(2- fluoroethyl)piperidin-4-yl]acetyl}amino)-19′,19′- dioxido-5′,21′,24′-trioxo-2-vinyl-1′,2′,3′,5′,6′,7′,8′, 9′,10′,11′,12′,13′,14′,20′,21′,23′,24′,24a′-octadeca- hydrospiro[cyclopropane-1,22′-pyrrolo[2,1- g][1,2,5,8,18]benzothiatetraazacycloicosin]-2′-yl 4-fluoro-1,3-dihydro-2H-isoindole-2-carboxylate | M+ = 882.3 MS method D | | 4.592 HPLC method A |
| 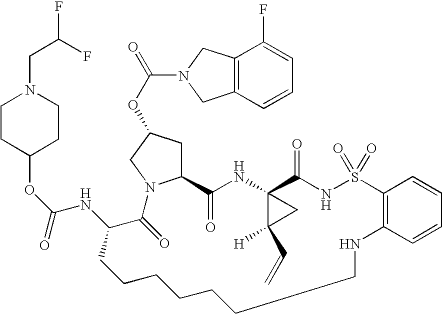 | Example 145: (1R,2S,2′R,6′S,24a′S)-6′-[({[1- (2,2-difluoroethyl)piperidin-4-yl]oxy}carbonyl) amino]-19′,19′-dioxido-5′,21′,24′-trioxo-2-vinyl- 1′,2′,3′,5′,6′,7′,8′,9′,10′,11′,12′,13′,14′,20′,21′,23′, 24′,24a′-octadecahydrospiro[cyclopropane- 1,22′-pyrrolo[2,1-g] [1,2,5,8,18]benzothiatetraazacycloicosin]-2′-yl 4-fluoro-1,3-dihydro-2H-isoindole-2-carboxylate | M+ = 902.2 MS method D | | 4.77 HPLC method A |
| 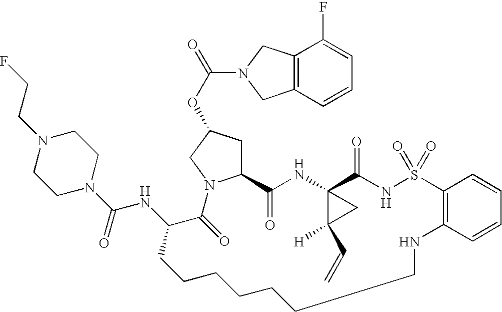 | Example 146: (1R,2S,2′R,6′S,24a′S)-6′-({[4-(2- fluoroethyl)piperazin-1-yl]carbonyl}amino)- 19′,19′-dioxido-5′,21′,24′-trioxo-2-vinyl-1′,2′,3′, 5′,6′,7′,8′,9′,10′,11′,12′,13′,14′,20′,21′,23′,24′,24a′- octadecahydrospiro[cyclopropane-1,22′-pyrrolo [2,1-g][1,2,5,8,18]benzothiatetraazacycloicosin]- 2′-yl 4-fluoro-1,3-dihydro-2H-isoindole-2- carboxylate | M+ = 869.2 MS method D | | 4.62 HPLC method A |
| 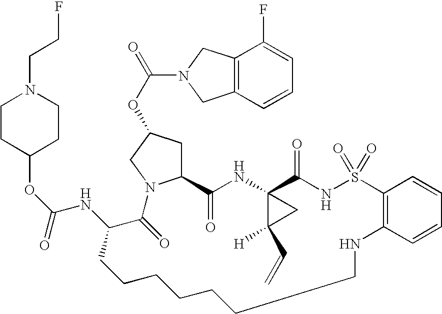 | Example 147: (1R,2S,2′R,6′S,24a′S)-6′-[({[1-(2- fluoroethyl)piperidin-4-yl]oxy}carbonyl)amino]- 19′,19′-dioxido-5′,21′,24′-trioxo-2-vinyl-1′,2′,3′,5′, 6′,7′,8′,9′,10′,11′,12′,13′,14′,20′,21′,23′,24′,24a′- octadecahydrospiro[cyclopropane-1,22′-pyrrolo [2,1-g][1,2,5,8,18]benzothiatetraazacycloicosin]- 2′-yl 4-fluoro-1,3-dihydro-2H-isoindole-2- carboxylate | M+ = 884.2 MS method D | | 4.71 HPLC method A |
| 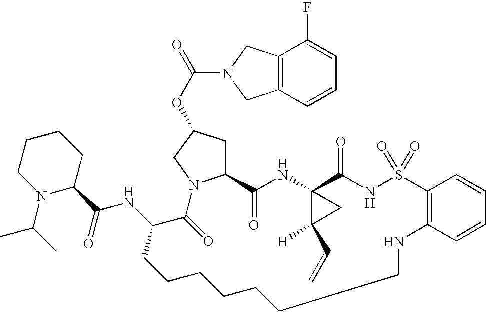 | Example 148: (1R,2S,2′R,6′S,24a′S)-6′-({[(2S)-1- isopropylpiperidin-2-yl]carbonyl}amino)-19′,19′- dioxido-5′,21′,24′-trioxo-2-vinyl-1′,2′,3′,5′,6′,7′,8′, 9′,10′,11′,12′,13′,14′,20′,21′,23′,24′,24a′- octadecahydrospiro[cyclopropane-1,22′-pyrrolo [2,1-g][1,2,5,8,18]benzothiatetraazacycloicosin]- 2′-yl 4-fluoro-1,3-dihydro-2H-isoindole-2- carboxylate | M+ = 864.2 MS method D | | 4.83 HPLC method A |
| 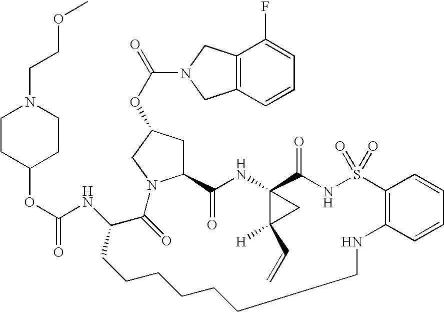 | Example 149: (1R,2S,2′R,6′S,24a′S)-6′-[({[1-(2- methoxyethyl)piperidin-4-yl]oxy}carbonyl) amino]-19′,19′-dioxido-5′,21′,24′-trioxo-2-vinyl- 1′,2′,3′,5′,6′,7′,8′,9′,10′,11′,12′,13′,14′,20′,21′,23′, 24′,24a′-octadecahydrospiro[cyclopropane- 1,22′-pyrrolo[2,1-g] [1,2,5,8,18]benzothiatetraazacycloicosin]-2′-yl 4-fluoro-1,3-dihydro-2H-isoindole-2-carboxylate | M+ = 896.2 MS method D | | 4.753 HPLC method A |
| 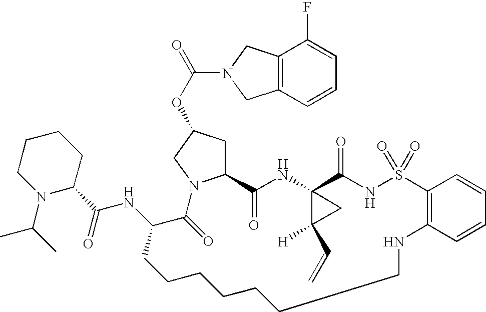 | Example 150: (1R,2S,2′R,6′S,24a′S)-6′-({[(2R)- 1-isopropylpiperidin-2-yl]carbonyl}amino)- 19′,19′-dioxido-5′,21′,24′-trioxo-2-vinyl-1′,2′,3′,5′, 6′,7′,8′,9′,10′,11′,12′,13′,14′,20′,21′,23′,24′,24a′- octadecahydrospiro[cyclopropane-1,22′-pyrrolo [2,1-g][1,2,5,8,18]benzothiatetraazacycloicosin]- 2′-yl 4-fluoro-1,3-dihydro-2H-isoindole-2- carboxylate | M+ = 864.2 MS method D | | 4.88 HPLC method A |
| 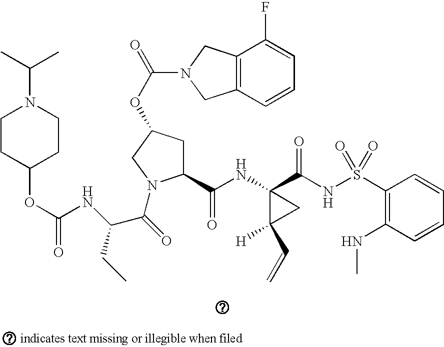 | Example 151: (1R,2S,2′R,6′S,24a′S)-6′-({[(1- isopropylpiperidin-4-yl)oxy]carbonyl}amino)- 19′,19′-dioxido-5′,21′,24′-trioxo-2-vinyl-1′,2′,3′,5′, 6′,7′,8′,9′,10′,11′,12′,13′,14′,20′,21′,23′,24′,24a′- octadecahydrospiro[cyclopropane-1,22′-pyrrolo [2,1-g][1,2,5,8,18]benzothiatetraazacycloicosin]- 2′-yl 4-fluoro-1,3-dihydro-2H-isoindole-2- carboxylate | M+ = 880.2 MS method D | | 4.848 HPLC method A |
| 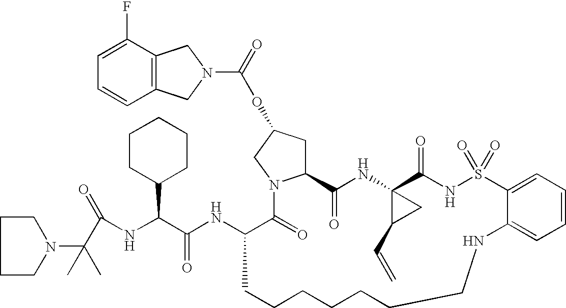 | Example 152: (1R,2S,2′R,6′S,24a′S)-6′-({(2S)-2- cyclohexyl-2-[(2-methyl-2-pyrrolidin-1-yl propanoyl)amino]acetyl}amino)-19′,19′-dioxido- 5′,21′,24′-trioxo-2-vinyl-1′,2′,3′,5′,6′,7′,8′,9′,10′, 11′,12′,13′,14′,20′,21′,23′,24′,24a′-octadeca- hydrospiro[cyclopropane-1,22′-pyrrolo[2,1- g][1,2,5,8,18]benzothiatetraazacycloicosin]-2′-yl 4-fluoro-1,3-dihydro-2H-isoindole-2-carboxylate | M+ = 990.0 MS method D | | 5.36 HPLC method A |
| 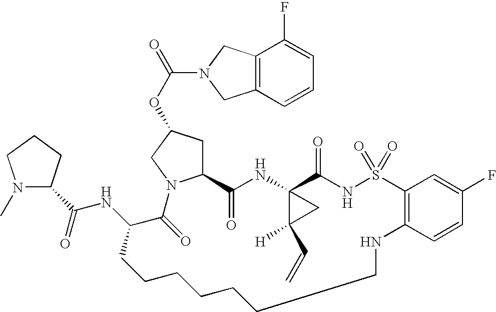 | Example 153: (1R,2S,2′R,6′S,24a′S)-17′-fluoro- 6′-[(1-methyl-D-prolyl)amino]-19′,19′-dioxido- 5′,21′,24′-trioxo-2-vinyl-1′,2′,3′,5′,6′,7′,8′,9′,10′, 11′,12′,13′,14′,20′,21′,23′,24′,24a′-octadecahydro- spiro[cyclopropane-1,22′-pyrrolo[2,1- g][1,2,5,8,18]benzothiatetraazacycloicosin]-2′-yl 4-fluoro-1,3-dihydro-2H-isoindole-2-carboxylate | M+ = 840.2 MS method D | | 4.82 HPLC method A |
| 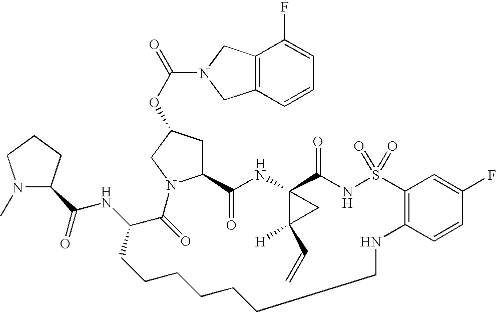 | Example 154: (1R,2S,2′R,6′S,24a′S)-17′-fluoro- 6′-[(1-methyl-L-prolyl)amino]-19′,19′-dioxido- 5′,21′,24′-trioxo-2-vinyl-1′,2′,3′,5′,6′,7′,8′,9′, 10′,11′,12′,13′,14′,20′,21′,23′,24′,24a′-octadeca- hydrospiro[cyclopropane-1,22′-pyrrolo[2,1- g][1,2,5,8,18]benzothiatetraazacycloicosin]-2′-yl 4-fluoro-1,3-dihydro-2H-isoindole-2-carboxylate | M+ = 840.2 MS method D | | 4.75 HPLC method A |
| 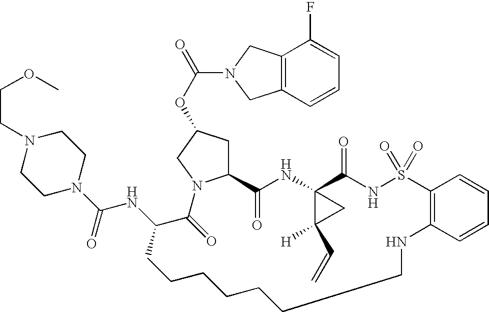 | Example 155: (1R,2S,2′R,6′S,24a′S)-6′-({[4-(2- methoxyethyl)piperazin-1-yl]carbonyl}amino)- 19′,19′-dioxido-5′,21′,24′-trioxo-2-vinyl-1′,2′,3′, 5′,6′,7′,8′,9′,10′,11′,12′,13′,14′,20′,21′,23′,24′,24a′- octadecahydrospiro[cyclopropane-1,22′-pyrrolo [2,1-g][1,2,5,8,18]benzothiatetraazacycloicosin]- 2′-yl 4-fluoro-1,3-dihydro-2H-isoindole-2- carboxylate | M+ = 881.2 MS method D | | 4.638 HPLC method A |
| 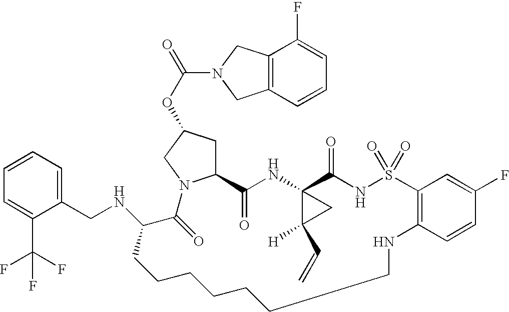 | Example 156: (1R,2S,2′R,6′S,24a′S)-17′-fluoro- 19′,19′-dioxido-5′,21′,24′-trioxo-6′-{[2- (trifluoromethyl)benzyl]amino}-2-vinyl-1′,2′,3′, 5′,6′,7′,8′,9′,10′,11′,12′,13′,14′,20′,21′,23′,24′,24a′- octadecahydrospiro[cyclopropane-1,22′-pyrrolo [2,1-g][1,2,5,8,18]benzothiatetraazacycloicosin]- 2′-yl 4-fluoro-1,3-dihydro-2H-isoindole-2- carboxylate | M+ = 887.2 MS method D | | 5.14 HPLC method A |
| 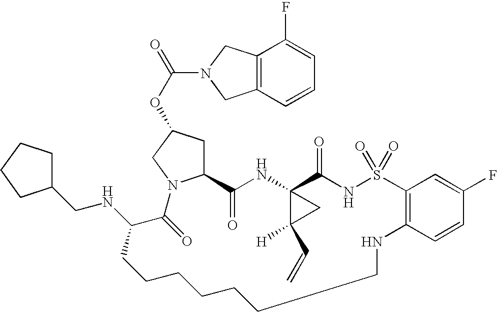 | Example 157: (1R,2S,2′R,6′S,24a′S)-6′- [(cyclopentylmethyl)amino]-17′-fluoro-19′,19′- dioxido-5′,21′,24′-trioxo-2-vinyl-1′,2′,3′,5′,6′,7′, 8′,9′,10′,11′,12′,13′,14′,20′,21′,23′,24′,24a′- octadecahydrospiro[cyclopropane-1,22′-pyrrolo [2,1-g][1,2,5,8,18]benzothiatetraazacycloicosin]- 2′-yl 4-fluoro-1,3-dihydro-2H-isoindole-2- carboxylate | M+ = 811.2 MS method D | | 5.00 HPLC method A |
| 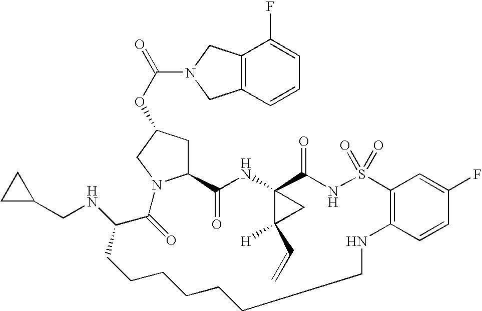 | Example 158: (1R,2S,2′R,6′S,24a′S)-6′- [(cyclopropylmethyl)amino]-17′-fluoro-19′,19′- dioxido-5′,21′,24′-trioxo-2-vinyl-1′,2′,3′,5′,6′, 7′,8′,9′,10′,11′,12′,13′,14′,20′,21′,23′,24′,24a′- octadecahydrospiro[cyclopropane-1,22′-pyrrolo [2,1-g][1,2,5,8,18]benzothiatetraazacycloicosin]- 2′-yl 4-fluoro-1,3-dihydro-2H-isoindole-2- carboxylate | M+ = 783.3 MS method D | | 4.84 HPLC method A |
| 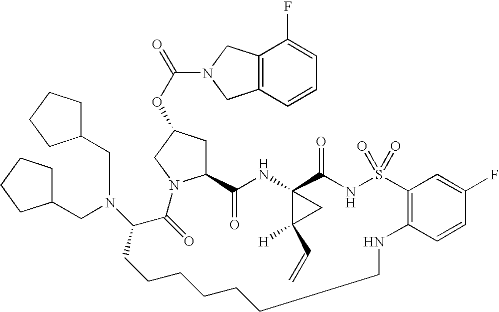 | Example 159: (1R,2S,2′R,6′S,24a′S)-6′- [bis(cyclopentylmethyl)amino]-17′-fluoro- 19′,19′-dioxido-5′,21′,24′-trioxo-2-vinyl-1′,2′,3′, 5′,6′,7′,8′,9′,10′,11′,12′,13′,14′,20′,21′,23′,24′,24a′- octadecahydrospiro[cyclopropane-1,22′-pyrrolo [2,1-g][1,2,5,8,18]benzothiatetraazacycloicosin]- 2′-yl 4-fluoro-1,3-dihydro-2H-isoindole-2- carboxylate | M+ = 893.3 MS method D | | 5.62 HPLC method A |
| 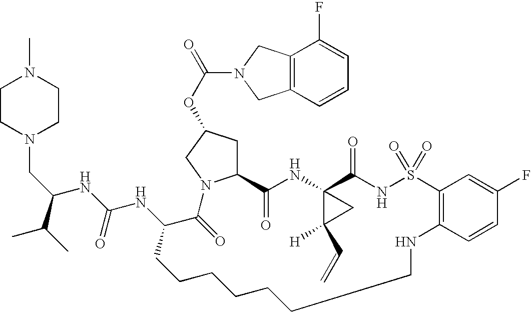 | Example 160: (1R,2S,2′R,6′S,24a′S)-17′-fluoro- 6′-[({(1S)-2-methyl-1-[(4-methylpiperazin-1- yl)methyl]propyl}carbamoyl)amino]-19′,19′- dioxido-5′,21′,24′-trioxo-2-vinyl-1′,2′,3′,5′,6′,7′,8′, 9′,10′,11′,12′,13′,14′,20′,21′,23′,24′,24a′- octadecahydrospiro[cyclopropane-1,22′-pyrrolo [2,1-g][1,2,5,8,18]benzothiatetraazacycloicosin]- 2′-yl 4-fluoro-1,3-dihydro-2H-isoindole-2- carboxylate | M+ = 940.3 MS method D | | 4.64 HPLC method A |
| 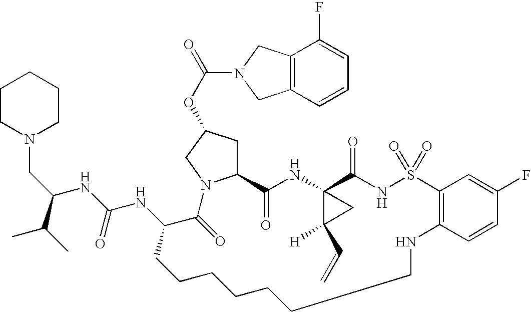 | Example 161: (1R,2S,2′R,6′S,24a′S)-17′-fluoro- 6′-({[(1S)-2-methyl-1-(piperidin-1-ylmethyl) propyl]carbamoyl}amino)-19′,19′-dioxido- 5′,21′,24′-trioxo-2-vinyl-1′,2′,3′,5′,6′,7′,8′,9′,10′, 11′,12′,13′,14′,20′,21′,23′,24′,24a′-octadecahydro- spiro[cyclopropane-1,22′-pyrrolo[2,1- g][1,2,5,8,18]benzothiatetraazacycloicosin]-2′-yl 4-fluoro-1,3-dihydro-2H-isoindole-2-carboxylate | M+ = 925.3 MS method D | | 5.15 HPLC method A |
| 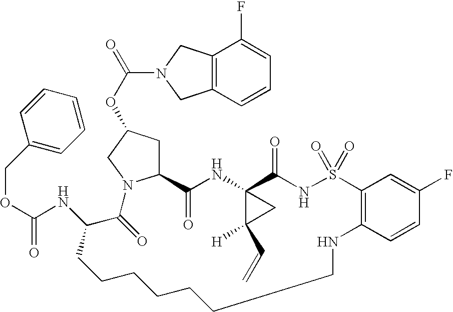 | Example 162: (1R,2S,2′R,6′S,24a′S)-6′- {[(benzyloxy)carbonyl]amino}-17′-fluoro- 19′,19′-dioxido-5′,21′,24′-trioxo-2-vinyl-1′,2′,3′,5′, 6′,7′,8′,9′,10′,11′,12′,13′,14′,20′,21′,23′,24′,24a′- octadecahydrospiro[cyclopropane-1,22′-pyrrolo [2,1-g][1,2,5,8,18]benzothiatetraazacycloicosin]- 2′-yl 4-fluoro-1,3-dihydro-2H-isoindole-2- carboxylate | M+ = 863.3 MS method D | | 5.86 HPLC method A |
| 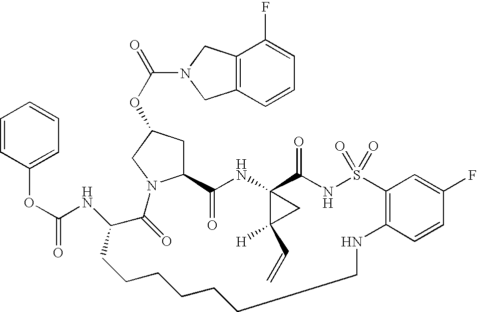 | Example 163: (1R,2S,2′R,6′S,24a′S)-17′-fluoro- 19′,19′-dioxido-5′,21′,24′-trioxo-6′-[(phenoxy carbonyl)amino]-2-vinyl-1′,2′,3′,5′,6′,7′,8′,9′,10′, 11′,12′,13′,14′,20′,21′,23′,24′,24a′-octadecahydro- spiro[cyclopropane-1,22′-pyrrolo[2,1- g][1,2,5,8,18]benzothiatetraazacycloicosin]-2′-yl 4-fluoro-1,3-dihydro-2H-isoindole-2-carboxylate | M+ = 849.2 MS method D | | 5.69 HPLC method A |
| 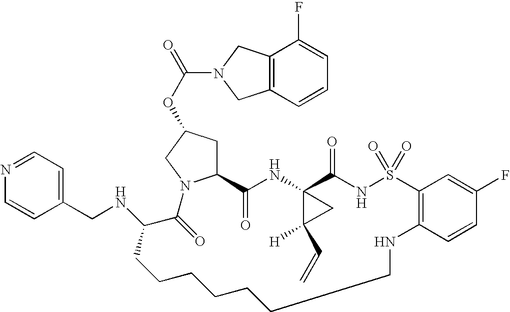 | Example 164: (1R,2S,2′R,6′S,24a′S)-17′-fluoro- 19′,19′-dioxido-5′,21′,24′-trioxo-6′-[(pyridin-4- ylmethyl)amino]-2-vinyl- 1′,2′,3′,5′,6′,7′,8′,9′,10′,11′,12′,13′,14′,20′,21′,23′, 24′,24a′-octadecahydrospiro[cyclopropane- 1,22′-pyrrolo[2,1-g] [1,2,5,8,18]benzothiatetraazacycloicosin]-2′-yl 4-fluoro-1,3-dihydro-2H-isoindole-2-carboxylate | M+ = 820.2 MS method D | | 4.50 HPLC method A |
| 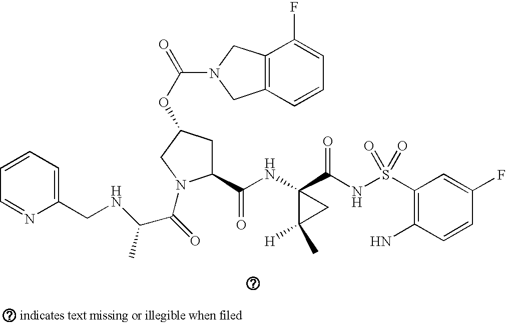 | Example 165: (1R,2S,2′R,6′S,24a′S)-17′-fluoro- 19′,19′-dioxido-5′,21′,24′-trioxo-6′-[(pyridin-2- ylmethyl)amino]-2-vinyl-1′,2′,3′,5′,6′,7′,8′,9′,10′, 11′,12′,13′,14′,20′,21′,23′,24′,24a′-octadecahydro- spiro[cyclopropane-1,22′-pyrrolo[2,1- g][1,2,5,8,18]benzothiatetraazacycloicosin]-2′-yl 4-fluoro-1,3-dihydro-2H-isoindole-2-carboxylate | M+ = 820.2 MS method D | | 4.75 HPLC method A |
| 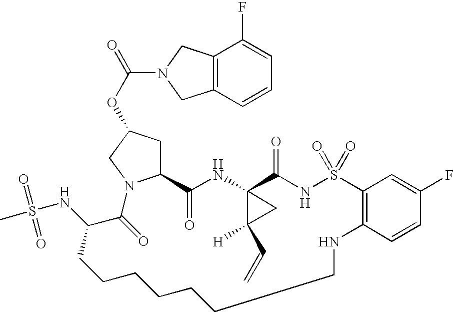 | Example 166: (1R,2S,2′R,6′S,24a′S)-17′-fluoro- 6′-[(methylsulfonyl)amino]-19′,19′-dioxido- 5′,21′,24′-trioxo-2-vinyl-1′,2′,3′,5′,6′,7′,8′,9′,10′, 11′,12′,13′,14′,20′,21′,23′,24′,24a′-octadecahydro- spiro[cyclopropane-1,22′-pyrrolo[2,1- g][1,2,5,8,18]benzothiatetraazacycloicosin]-2′-yl 4-fluoro-1,3-dihydro-2H-isoindole-2-carboxylate | M+ = 807.0 MS method D | | 5.17 HPLC method A |
| 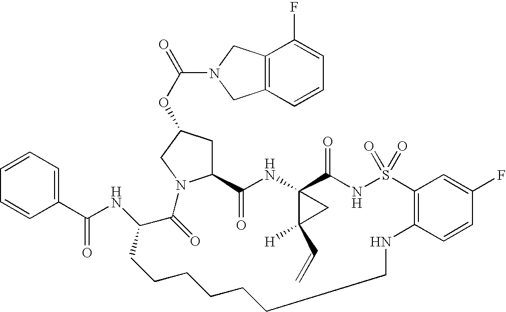 | Example 167: (1R,2S,2′R,6′S,24a′S)-6′- (benzoylamino)-17′-fluoro-19′,19′-dioxido- 5′,21′,24′-trioxo-2-vinyl-1′,2′,3′,5′,6′,7′,8′,9′,10′, 11′,12′,13′,14′,20′,21′,23′,24′,24a′-octadecahydro- spiro[cyclopropane-1,22′-pyrrolo[2,1- g][1,2,5,8,18]benzothiatetraazacycloicosin]-2′-yl 4-fluoro-1,3-dihydro-2H-isoindole-2-carboxylate | M+ = 833.2 MS method D | | 5.66 HPLC method A |
| 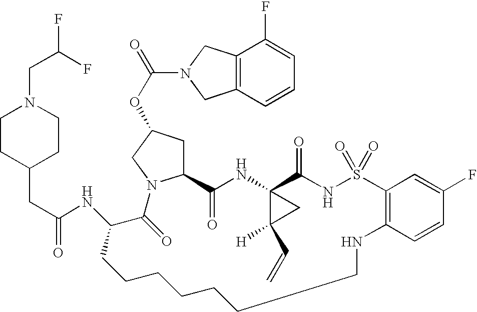 | Example 168: (1R,2S,2′R,6′S,24a′S)-6′-({[1-(2,2- difluoroethyl)piperidin-4-yl]acetyl}amino)-17′- fluoro-19′,19′-dioxido-5′,21′,24′-trioxo-2-vinyl- 1′,2′,3′,5′,6′,7′,8′,9′,10′,11′,12′,13′,14′,20′,21′,23′, 24′,24a′-octadecahydrospiro[cyclopropane-1,22′- pyrrolo[2,1-g][1,2,5,8,18]benzothiatetraaza- cycloicosin]-2′-yl 4-fluoro-1,3-dihydro-2H- isoindole-2-carboxylate | M+ = 918.3 MS method D | | 4.780 HPLC method A |
| 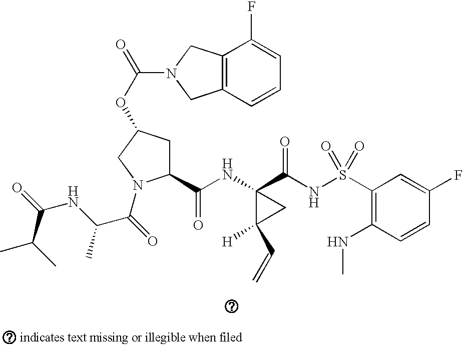 | Example 169: (1R,2S,2′R,6′S,24a′S)-6′-({[(3R)- 1-ethylpiperidin-3-yl]carbonyl}amino)-17′- fluoro-19′,19′-dioxido-5′,21′,24′-trioxo-2-vinyl- 1′,2′,3′,5′,6′,7′,8′,9′,10′,11′,12′,13′,14′,20′,21′,23′, 24′,24a′-octadecahydrospiro[cyclopropane-1,22′- pyrrolo[2,1-g][1,2,5,8,18]benzothiatetraaza- cycloicosin]-2′-yl 4-fluoro-1,3-dihydro-2H- isoindole-2-carboxylate | M+ = 868.2 MS method D | | 4.81 HPLC method A |
| 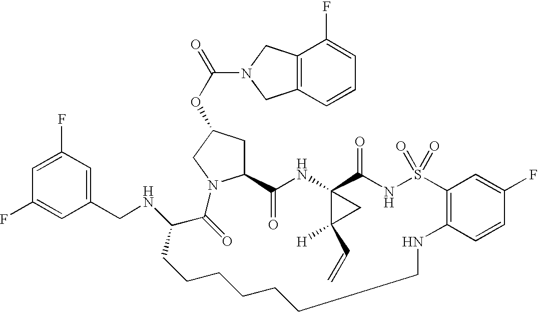 | Example 170: (1R,2S,2′R,6′S,24a′S)-6′-[(3,5- difluorobenzyl)amino]-17′-fluoro-19′,19′- dioxido-5′,21′,24′-trioxo-2-vinyl- 1′,2′,3′,5′,6′,7′,8′,9′,10′,11′,12′,13′,14′,20′,21′,23′, 24′,24a′-octadecahydrospiro[cyclopropane-1,22′- pyrrolo[2,1-g][1,2,5,8,18]benzothiatetraaza- cycloicosin]-2′-yl 4-fluoro-1,3-dihydro-2H- isoindole-2-carboxylate | M+ = 855.3 MS method D | | 5.10 HPLC method A |
| 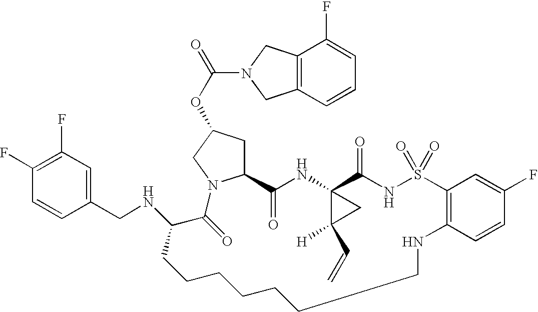 | Example 171: (1R,2S,2′R,6′S,24a′S)-6′-[(3,4- difluorobenzyl)amino]-17′-fluoro-19′,19′- dioxido-5′,21′,24′-trioxo-2-vinyl-1′,2′,3′,5′,6′,7′, 8′,9′,10′,11′,12′,13′,14′,20′,21′,23′,24′,24a′- octadecahydrospiro[cyclopropane-1,22′-pyrrolo [2,1-g][1,2,5,8,18]benzothiatetraazacycloicosin]- 2′-yl 4-fluoro-1,3-dihydro-2H-isoindole-2- carboxylate | M+ = 855.3 MS method D | | 5.03 HPLC method A |
| 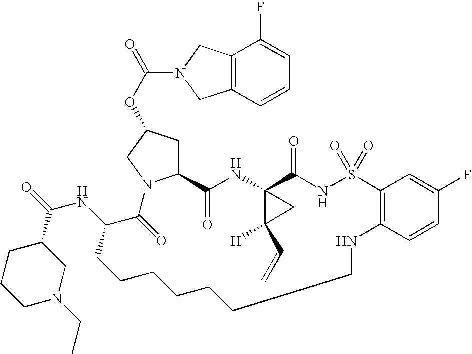 | Example 172: (1R,2S,2′R,6′S,24a′S)-6′-({[(3S)-1- ethylpiperidin-3-yl]carbonyl}amino)-17′-fluoro- 19′,19′-dioxido-5′,21′,24′-trioxo-2-vinyl-1′,2′,3′,5′, 6′,7′,8′,9′,10′,11′,12′,13′,14′,20′,21′,23′,24′,24a′- octadecahydrospiro[cyclopropane-1,22′-pyrrolo [2,1-g][1,2,5,8,18]benzothiatetraazacycloicosin]- 2′-yl 4-fluoro-1,3-dihydro-2H-isoindole-2- carboxylate | M+ = 868.2 MS method D | | 4.91 HPLC method A |
| 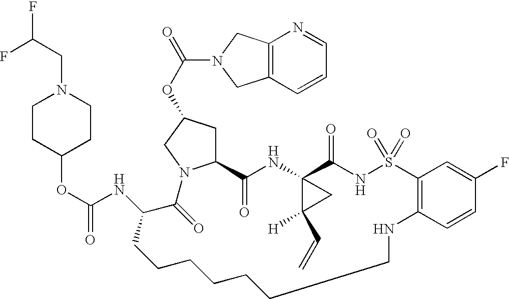 | Example 173: (1R,2S,2′R,6′S,24a′S)-6′-[({[1- (2,2-difluoroethyl)piperidin-4-yl]oxy}carbonyl) amino]-17′-fluoro-19′,19′-dioxido-5′,21′,24′- trioxo-2-vinyl-1′,2′,3′,5′,6′,7′,8′,9′,10′,11′,12′, 13′,14′,20′,21′,23′,24′,24a′-octadecahydro- spiro[cyclopropane-1,22′-pyrrolo[2,1-g] [1,2,5,8,18]benzothiatetraazacycloicosin]-2′-yl 5,7-dihydro-6H-pyrrolo[3,4-b]pyridine-6- carboxylate | M+ = 903.3 MS method D | | 4.427 HPLC method A |
| 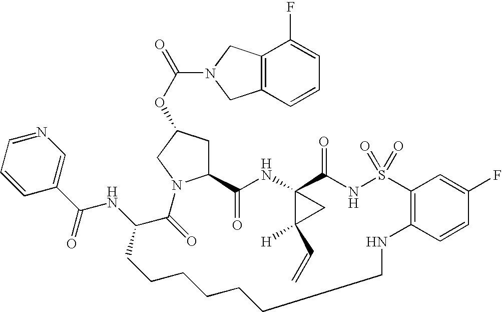 | Example 174: (1R,2S,2′R,6′S,24a′S)-17′-fluoro- 19′,19′-dioxido-5′,21′,24′-trioxo-6′-[(pyridin-3- ylcarbonyl)amino]-2-vinyl-1′,2′,3′,5′,6′,7′,8′, 9′,10′,11′,12′,13′,14′,20′,21′,23′,24′,24a′-octadeca- hydrospiro[cyclopropane-1,22′-pyrrolo[2,1- g][1,2,5,8,18]benzothiatetraazacycloicosin]-2′-yl 4-fluoro-1,3-dihydro-2H-isoindole-2-carboxylate | M+ = 834.2 MS method D | | 3.07 HPLC method A |
| 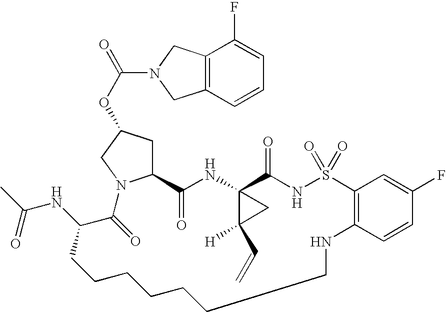 | Example 175: (1R,2S,2′R,6′S,24a′S)-6′- acetamido-17′-fluoro-19′,19′-dioxido-5′,21′,24′- trioxo-2-vinyl-1′,2′,3′,5′,6′,7′,8′,9′,10′,11′,12′, 13′,14′,20′,21′,23′,24′,24a′-octadecahydro- spiro[cyclopropane-1,22′-pyrrolo[2,1- g][1,2,5,8,18]benzothiatetraazacycloicosin]-2′-yl 4-fluoro-1,3-dihydro-2H-isoindole-2-carboxylate | M+ = 771.3 MS method D | | 5.10 HPLC method A |
| 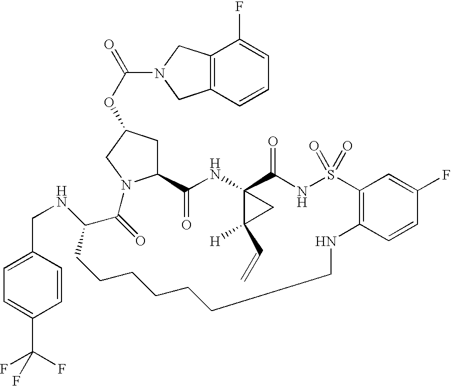 | Example 176: (1R,2S,2′R,6′S,24a′S)-17′-fluoro- 19′,19′-dioxido-5′,21′,24′-trioxo-6′-{[4- (trifluoromethyl)benzyl]amino}-2-vinyl-1′,2′,3′, 5′,6′,7′,8′,9′,10′,11′,12′,13′,14′,20′,21′,23′,24′,24a′- octadecahydrospiro[cyclopropane-1,22′-pyrrolo [2,1-g][1,2,5,8,18]benzothiatetraazacycloicosin]- 2′-yl 4-fluoro-1,3-dihydro-2H-isoindole-2- carboxylate | M+ = 887.2 MS method D | | 3.38 HPLC method A |
| 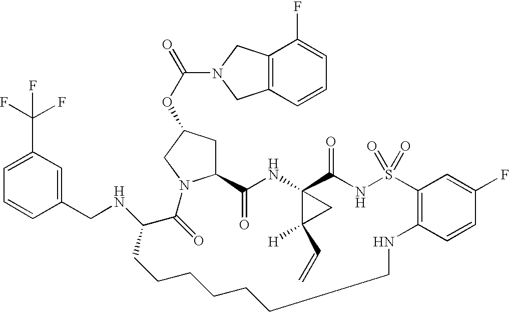 | Example 177: (1R,2S,2′R,6′S,24a′S)-17′-fluoro- 19′,19′-dioxido-5′,21′,24′-trioxo-6′-{[3- (trifluoromethyl)benzyl]amino}-2-vinyl-1′,2′,3′, 5′,6′,7′,8′,9′,10′,11′,12′,13′,14′,20′,21′,23′,24′,24a′- octadecahydrospiro[cyclopropane-1,22′-pyrrolo [2,1-g][1,2,5,8,18]benzothiatetraazacycloicosin]- 2′-yl 4-fluoro-1,3-dihydro-2H-isoindole-2- carboxylate | M+ = 887.2 MS method D | | 3.04 HPLC method A |
| 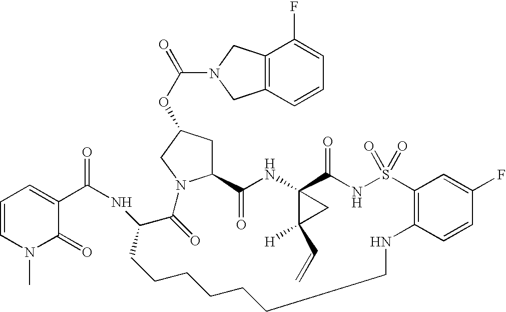 | Example 178: (1R,2S,2′R,6′S,24a′S)-17′-fluoro- 6′-{[(1-methyl-2-oxo-1,2-dihydropyridin-3-yl) carbonyl]amino}-19′,19′-dioxido-5′,21′,24′- trioxo-2-vinyl-1′,2′,3′,5′,6′,7′,8′,9′,10′,11′,12′,13′, 14′,20′,21′,23′,24′,24a′-octadecahydrospiro [cyclopropane-1,22′-pyrrolo[2,1- g][1,2,5,8,18]benzothiatetraazacycloicosin]-2′-yl 4-fluoro-1,3-dihydro-2H-isoindole-2-carboxylate | M+ = 864.2 MS method D | | 3.628 HPLC method A |
| 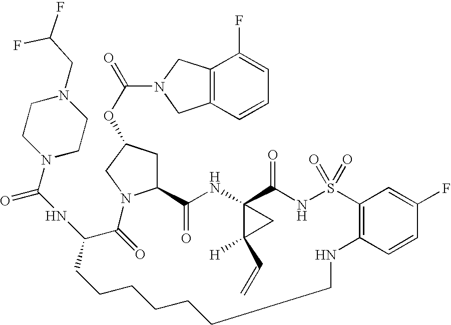 | Example 179: (1R,2S,2′R,6′S,24a′S)-6′-({[4-(2,2- difluoroethyl)piperazin-1-yl]carbonyl}amino)- 17′-fluoro-19′,19′-dioxido-5′,21′,24′-trioxo-2- vinyl-1′,2′,3′,5′,6′,7′,8′,9′,10′,11′,12′,13′,14′,20′, 21′,23′,24′,24a′-octadecahydrospiro [cyclopropane-1,22′-pyrrolo[2,1-g][1,2,5,8,18] benzothiatetraazacycloicosin]-2′-yl 4-fluoro-1,3- dihydro-2H-isoindole-2-carboxylate | M+ = 905.2 MS method D | | 3.07 HPLC method A |
| 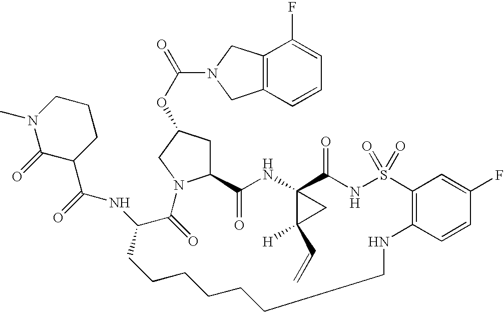 | Example 180: (1R,2S,2′R,6′S,24a′S)-17′-fluoro- 6′-{[(1-methyl-2-oxopiperidin-3-yl)carbonyl] amino}-19′,19′-dioxido-5′,21′,24′-trioxo-2-vinyl- 1′,2′,3′,5′,6′,7′,8′,9′,10′,11′,12′,13′,14′,20′,21′,23′, 24′,24a′-octadecahydrospiro[cyclopropane-1,22′- pyrrolo[2,1-g][1,2,5,8,18]benzothiatetraaza- cycloicosin]-2′-yl 4-fluoro-1,3-dihydro-2H- isoindole-2-carboxylate (2 diastereoisomers) | M+ = 868.2 MS method D | | 3.21 (Isomer A) 3.42 (Isomer B) HPLC method A |
| 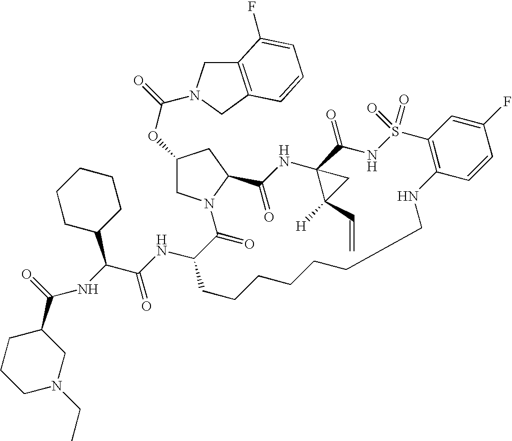 | Example 181: (1R,2S,2′R,6′S,24a′S)-6′-{[(2S)-2- cyclohexyl-2-({[(3R)-1-ethylpiperidin-3-yl] carbonyl}amino)acetyl]amino}-17′-fluoro- 19′,19′-dioxido-5′,21′,24′-trioxo-2-vinyl-1′,2′,3′,5′, 6′,7′,8′,9′,10′,11′,12′,13′,14′,20′,21′,23′,24′,24a′- octadecahydrospiro[cyclopropane-1,22′-pyrrolo [2,1-g][1,2,5,8,18]benzothiatetraazacycloicosin]- 2′-yl 4-fluoro-1,3-dihydro-2H-isoindole-2- carboxylate | M+ = 1007.5 MS method D | | 3.51 HPLC method A |
| 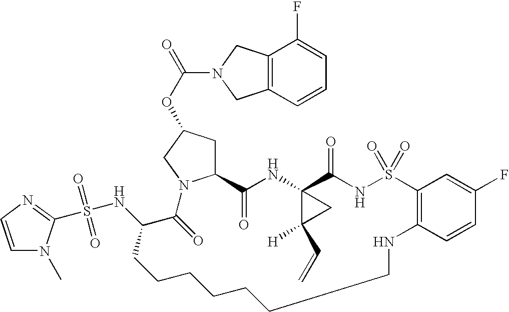 | Example 182: (1R,2S,2′R,6′S,24a′S)-17′-fluoro- 6′-{[(1-methyl-1H-imidazol-2-yl)sulfonyl] amino}-19′,19′-dioxido-5′,21′,24′-trioxo-2-vinyl- 1′,2′,3′,5′,6′,7′,8′,9′,10′,11′,12′,13′,14′,20′,21′,23′, 24′,24a-octadecahydrospiro[cyclopropane-1,22′- pyrrolo[2,1-g][1,2,5,8,18]benzothiatetraaza- cycloicosin]-2′-yl 4-fluoro-1,3-dihydro-2H- isoindole-2-carboxylate | M+ = 873.3 MS method D | | 5.96 HPLC method A |
| 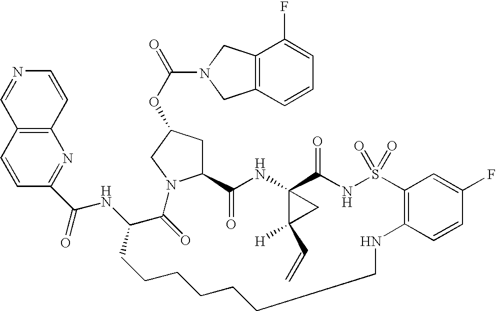 | Example 183: (1R,2S,2′R,6′S,24a′S)-17′-fluoro- 6′-[(1,6-naphthyridin-2-ylcarbonyl)amino]- 19′,19′-dioxido-5′,21′,24′-trioxo-2-vinyl-1′,2′,3′,5′, 6′,7′,8′,9′,10′,11′,12′,13′,14′,20′,21′,23′,24′,24a′- octadecahydrospiro[cyclopropane-1,22′-pyrrolo [2,1-g][1,2,5,8,18]benzothiatetraazacycloicosin]- 2′-yl 4-fluoro-1,3-dihydro-2H-isoindole-2- carboxylate | M+ = 585.3 MS method D | | 5.71 HPLC method A |
| 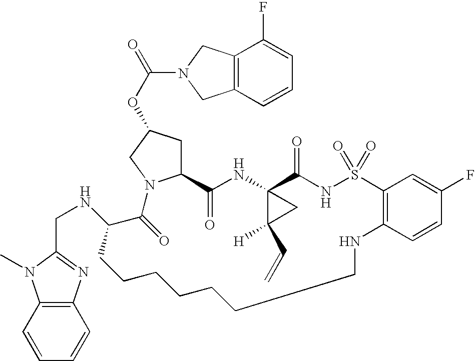 | Example 184: (1R,2S,2′R,6′S,24a′S)-17′-fluoro- 6′-{[(1-methyl-1H-benzimidazol-2-yl)methyl] amino}-19′,19′-dioxido-5′,21′,24′-trioxo-2-vinyl- 1′,2′,3′,5′,6′,7′,8′,9′,10′,11′,12′,13′,14′,20′,21′,23′, 24′,24a′-octadecahydrospiro[cyclopropane-1,22′- pyrrolo[2,1-g][1,2,5,8,18]benzothiatetraaza- cycloicosin]-2′-yl 4-fluoro-1,3-dihydro-2H- isoindole-2-carboxylate | M+ = 873.3 MS method D | | 5.33 HPLC method A |
| 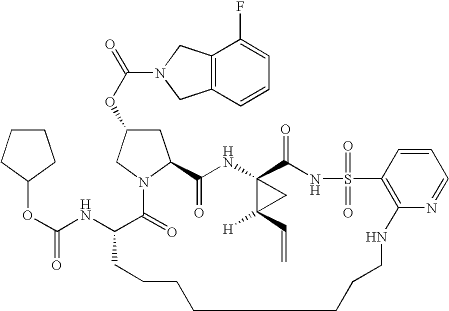 | Example 185: (1R,2S,13′S,17′R,18a′S)-13′- {[(cyclopentyloxy)carbonyl]amino}-24′,24′- dioxido-14′,19′,22′-trioxo-2-vinyl- 5′,6′,7′,8′,9′,10′,11′,12′,13′,14′,16′,17′,18′,18a′,19′, 20′,22′,23′-octadecahydrospiro[cyclopropane- 1,21′-pyrido[2,3-s]pyrrolo[2,1- g][1,2,5,8,18]thiatetraazacycloicosin]-17′-yl 4- fluoro-1,3-dihydro-2H-isoindole-2-carboxylate | | 4.136; M + H = 824.2 LC MS method E | 4.179 HPLC method C |
| 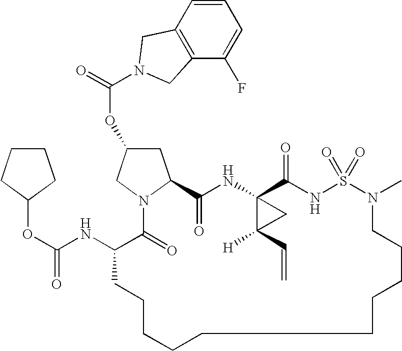 | Example 186: (1R,2S,18′S,22′R,23a′S)-18′- {[(cyclopentyloxy)carbonyl]amino}-7′-methyl- 6′,6′-dioxido-1,4′,19′-trioxo-2-vinylicosahydro- 7′H-spiro[cyclopropane-1,3′-pyrrolo[2,1- g][1,2,5,8,21]thiatetraazacyclohenicosin]-22′-yl 4-fluoro-1,3-dihydro-2H-isoindole-2-carboxylate | | 4.676; M + H = 803.3 LC MS method E | 4.555 HPLC method C |
| 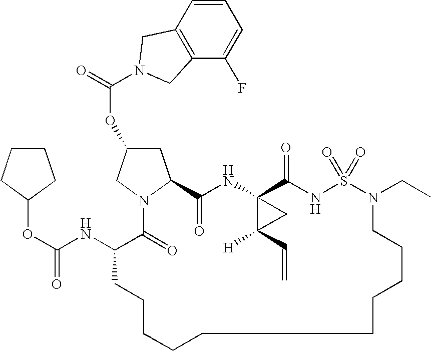 | Example 187: (1R,2S,18′S,22′R,23a′S)-18′- {[(cyclopentyloxy)carbonyl]amino}-7′-ethyl- 6′,6′-dioxido-1′,4′,19′-trioxo-2-vinylicosahydro- 7′H-spiro[cyclopropane-1,3′-pyrrolo[2,1- g][1,2,5,8,21]thiatetraazacyclohenicosin]-22′-yl 4-fluoro-1,3-dihydro-2H-isoindole-2-carboxylate | | 4.875; M + H = 817.3 LC MS method E | 4.652 HPLC method C |
| 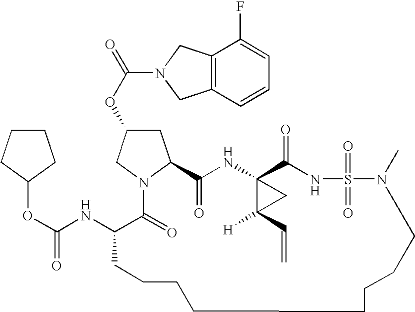 | Example 188: (1R,2S,16′S,20′R,21a′S)-16′- {[(cyclopentyloxy)carbonyl]amino}-7′-methyl- 6′,6′-dioxido-1′,4′,17′-trioxo-2- vinyloctadecahydro-7′H-spiro[cyclopropane- 1,3′-pyrrolo[2,1-g] [1,2,5,8,19]thiatetraazacyclononadecin]-20′-yl 4-fluoro-1,3-dihydro-2H-isoindole-2-carboxylate | | 4.330; M + H = 775.3 LC MS method E | 4.419 HPLC method C |
| 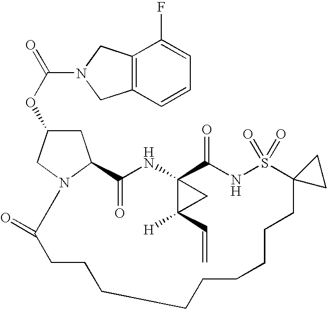 | Example 189: (1R,2S,20′R,21a′S)-6′,6′-dioxido- 1′,4′,17′-trioxo-2- vinyloctadecahydrodispiro[cyclopropane- 1,3′-pyrrolo[2,1-g] [1,2,5,8]thiatriazacyclononadecine-7′,1″- cyclopropan]-20′-yl 4-fluoro-1,3-dihydro-2H- isoindole-2-carboxylate | | 4.281; M + H = 659.0 LC MS method E | 4.072 HPLC method C |
| 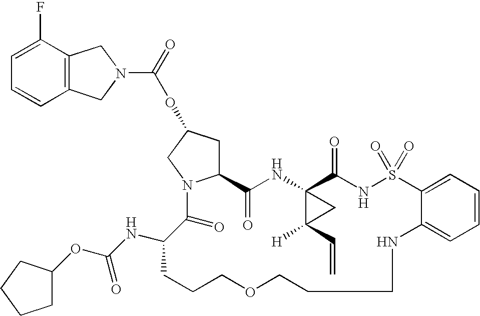 | Example 190: (1R,2S,2′R,6′S,24a′S)-6′- {[(cyclopentyloxy)carbonyl]amino}-19′,19′- dioxido-5′,21′,24′-trioxo-2-vinyl- 1′,2′,3′,6′,7′,8′,9′,11′,12′,13′,14′,20′,21′,23′,24′,24a′- hexadecahydro-5′H-spiro[cyclopropane- 1,22′-pyrrolo[2,1-g] [14,1,2,5,8,18]benzoxathiatetraazacycloicosin]- 2′-yl 4-fluoro-1,3-dihydro-2H-isoindole-2- carboxylate | M+ = 825.3 MS method D | | 5.284 HPLC method A |
| 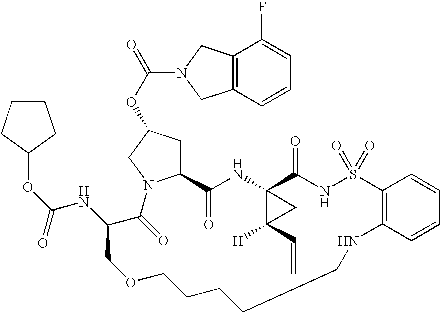 | Example 191: (1R,2S,2′R,6′R,24a′S)-6′- {[(cyclopentyloxy)carbonyl]amino}-19′,19′- dioxido-5′,21′,24′-trioxo-2-vinyl-1′,2′,3′,6′, 7′,9′,10′,11′,12′,13′,14′,20′,21′,23′,24′,24a′- hexadecahydro-5′H-spiro[cyclopropane-1,22′- pyrrolo[2,1-g][12,1,2,5,8,18] benzoxathiatetraazacycloicosin]- 2′-yl 4-fluoro-1,3-dihydro-2H-isoindole-2- carboxylate | M+ = 825.3 MS method D | | 5.63 HPLC method A |
| 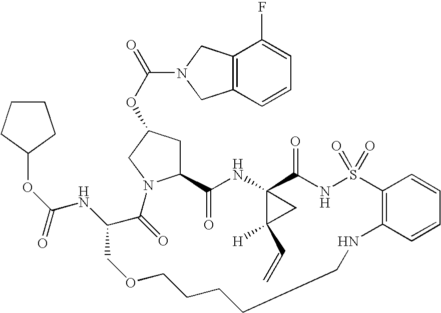 | Example 192: (1R,2S,2′R,6′S,24a′S)-6′- {[(cyclopentyloxy)carbonyl]amino}-19′,19′- dioxido-5′,21′,24′-trioxo-2-vinyl- 1′,2′,3′,6′,7′,9′,10′,11′,12′,13′,14′,20′,21′,23′,24′, 24a′-hexadecahydro-5′H-spiro[cyclopropane- 1,22′-pyrrolo[2,1-g] [12,1,2,5,8,18]benzoxathiatetraazacycloicosin]- 2′-yl 4-fluoro-1,3-dihydro-2H-isoindole-2- carboxylate | M+ = 825.3 MS method D | | 5.45 HPLC method A |
| 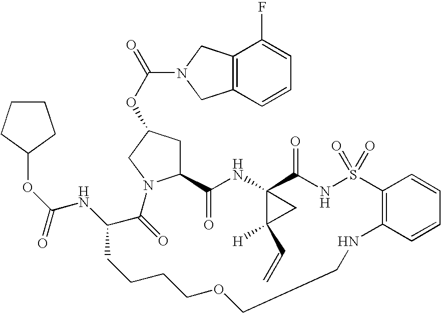 | Example 193: (1R,2S,2′R,6′S,24a′S)-6′- {[(cyclopentyloxy)carbonyl]amino}-19′,19′- dioxido-5′,21′,24′-trioxo-2-vinyl- 1′,2′,3′,5′,6′,7′,8′,9′,10′,13′,14′,20′,21′,23′,24′,24a′- hexadecahydro-12′H-spiro[cyclopropane- 1,22′-pyrrolo[2,1-g] [15,1,2,5,8,18]benzoxathiatetraazacycloicosin]- 2′-yl 4-fluoro-1,3-dihydro-2H-isoindole-2- carboxylate | M + H = 825.3 MS method D | | 5.28 HPLC method A |
| 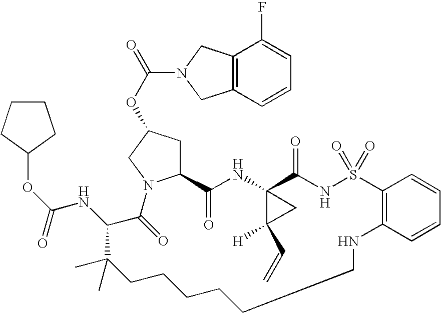 | Example 194: (1R,2S,2′R,6′S,24a′S)-6′- {[(cyclopentyloxy)carbonyl]amino}-7′,7′- dimethyl-19′,19′-dioxido-5′,21′,24′-trioxo-2- vinyl-1′,2′,3′,5′,6′, 7′,8′,9′,10′,11′,12′,13′,14′,20′,21′,23′, 24′,24a′-octadecahydrospiro[cyclopropane- 1,22′-pyrrolo[2,1-g] [1,2,5,8,18]benzothiatetraazacycloicosin]-2′-yl 4-fluoro-1,3-dihydro-2H-isoindole-2-carboxylate | M+ = 851.2 MS method D | | 6.42 HPLC method A |
| 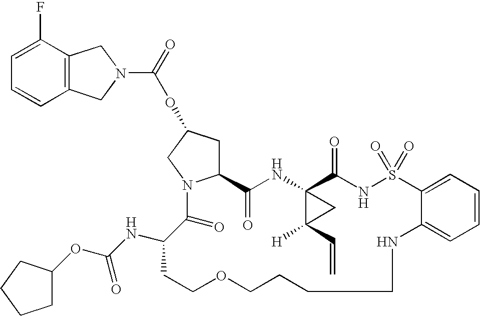 | Example 195: (1R,2S,2′R,6′S,24a′S)-6′- {[(cyclopentyloxy)carbonyl]amino}-19′,19′- dioxido-5′,21′,24′-trioxo-2-vinyl- 1′,2′,3′,5′,6′,7′,8′,11′,12′,13′,14′,20′,21′,23′,24′,24a′- hexadecahydro-10′H-spiro[cyclopropane- 1,22′-pyrrolo[2,1-g] [13,1,2,5,8,18]benzoxathiatetraazacycloicosin]- 2′-yl 4-fluoro-1,3-dihydro-2H-isoindole-2- carboxylate | M+ = 825.3 MS method D | | 5.265 HPLC method A |
| 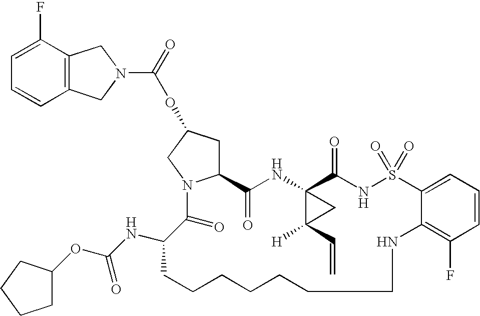 | Example 196: (1R,2S,2′R,6′S,24a′S)-6′- {[(cyclopentyloxy)carbonyl]amino}-15′-fluoro- 19′,19′-dioxido-5′,21′,24′-trioxo-2-vinyl- 1′,2′,3′,5′,6′,7′,8′,9′,10′,11′,12′,13′,14′,20′,21′,23′, 24′,24a′-octadecahydrospiro[cyclopropane- 1,22′-pyrrolo[2,1-g] [1,2,5,8,18]benzothiatetraazacycloicosin]-2′-yl 4-fluoro-1,3-dihydro-2H-isoindole-2-carboxylate | M+ = 841.3 MS method D M+ = 801.2 MS method D | | 6.120 HPLC method A 4.88 HPLC method A |
| 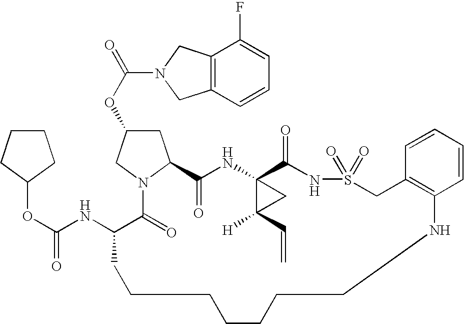 | Example 197: (1R,2S,2′R,6′S,25a′S)-6′- {[(cyclopentyloxy)carbonyl]amino}-20′,20′- dioxido-5′,22′,25′-trioxo-2-vinyl- 1′,2′,3′,6′,7′,8′,9′,10′,11′,12′,13′,14′,19′,21′,22′,24′, 25′,25a′-octadecahydro-5′H-spiro[cyclopropane- 1,23′-pyrrolo[1,2-k] [18,1,11,14,17]benzothiatetraazacycloheni- cosin]-2′-yl 4-fluoro-1,3-dihydro-2H-isoindole-2- carboxylate | M+ = 837.2 MS method D | | 5.63 HPLC method A |
| 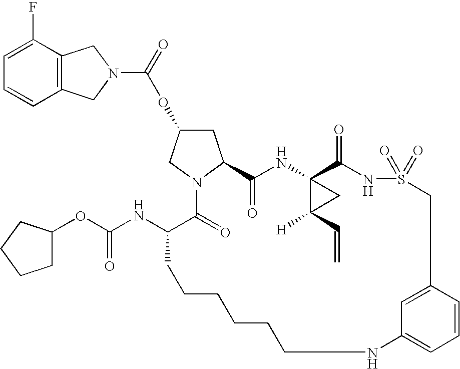 | Example 198: (1R,2S,20′S,24′R,25a′S)-20′- {[(cyclopentyloxy)carbonyl]amino}-6′,6′- dioxido-1′,4′,21′-trioxo-2-vinyl- 1′H,2′H,4′H,5′H,7′H,13′H,14′H,15′H,16′H,17′H, 18′H,19′H,20′H,21′H,23′H,24′H,25′H,25a′H- spiro[cyclopropane-1,3′- [6]thia[2,5,13,22]tetraaza[12,8](metheno)pyrrolo [2,1-g][1,2,5,8,17]thiatetraazacyclotricosin]-24′- yl 4-fluoro-1,3-dihydro-2H-isoindole-2- carboxylate | M+ = 823.3 MS method D | | 4.52 HPLC method A |
| 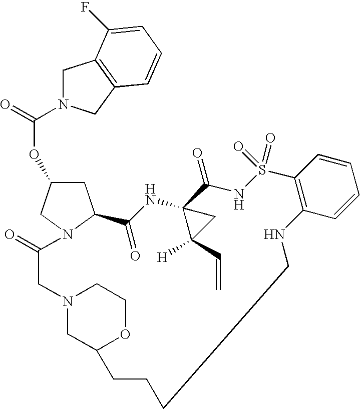 | Example 199: (1R,2S,2′R,26a′S)-21′,21′-dioxido- 5′,23′,26′-trioxo-2-vinyl- 1′,2′,3′,5′,6′,8′,9′,11′,12′,13′,14′,15′,16′,22′,23′,25′, 26′,26a′-octadecahydrospiro[cyclopropane-1,24′- [10]oxa[21]thia[4,7,16,22,25]pentaaza[7,11] methanopyrrolo[2,1-g] [14,1,2,5,8,11,20]benzoxathiapentaaza- cyclodocosin]-2′-yl 4-fluoro-1,3-dihydro-2H- isoindole-2-carboxylate | M+ = 739.2 MS method D | | 4.28 HPLC method A |
| 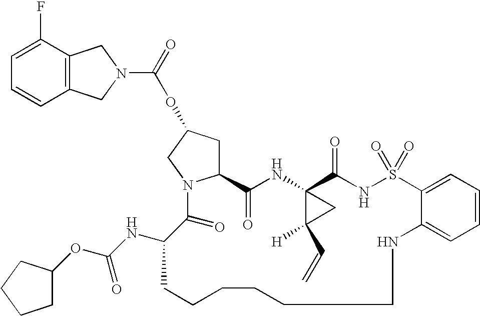 | Example 200: (1R,2S,2′R,6′S,23a′S)-6′- {[(cyclopentyloxy)carbonyl]amino}-18′,18′- dioxido-5′,20′,23′-trioxo-2-vinyl- 1′,2′,3′,6′,7′,8′,9′,10′,11′,12′,13′,19′,20′,22′,23′,23a′- hexadecahydro-5′H-spiro[cyclopropane- 1,21′-pyrrolo[2,1-g] [1,2,5,8,17]benzothiatetraazacyclononadecin]- 2′-yl 4-fluoro-1,3-dihydro-2H-isoindole-2- carboxylate | M+ = 809.2 MS method D | | 5.748 HPLC method A |
| 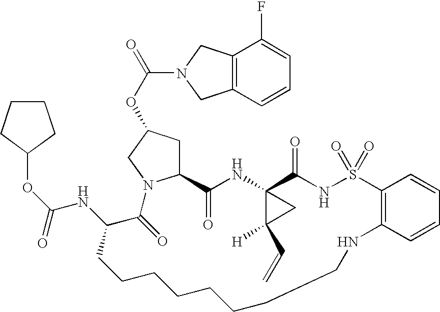 | Example 201: (1R,2S,2′R,6′S,25a′S)-6′- {[(cyclopentyloxy)carbonyl]amino}-20′,20′- dioxido-5′,22′,25′-trioxo-2-vinyl- 1′,2′,3′,6′,7′,8′,9′,10′,11′,12′,13′,14′,15′,21′,22′,24′, 25′,25a′-octadecahydro-5′H-spiro[cyclopropane- 1,23′-pyrrolo[2,1-g] [1,2,5,8,19]benzothiatetraazacyclohenicosin]- 2′-yl 4-fluoro-1,3-dihydro-2H-isoindole-2- carboxylate | M+ = 837.2 MS method D | | 6.14 HPLC method A |
| 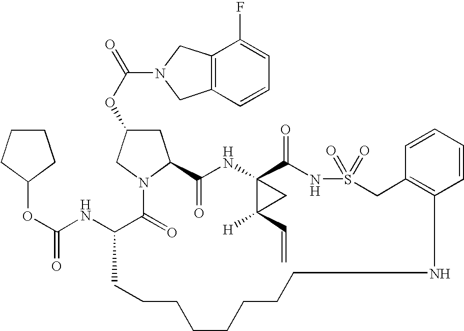 | Example 202: (1R,2S,2′R,6′S,26a′S)-6′- {[(cyclopentyloxy)carbonyl]amino}-21′,21′- dioxido-5′,23′,26′-trioxo-2-vinyl- 1′,2′,3′,5′,6′,7′,8′,9′,10′,11′,12′,13′,14′,15′,20′,22′, 23′,25′,26′,26a′-icosahydrospiro[cyclopropane- 1,24′-pyrrolo[1,2-l] [19,1,12,15,18]benzothiatetraazacyclodocosin]- 2′-yl 4-fluoro-1,3-dihydro-2H-isoindole-2- carboxylate | M+ = 851.2 MS method D | | 5.89 HPLC method A |
| 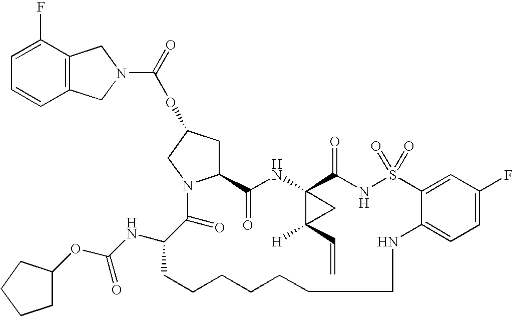 | Example 203: (1R,2S,2′R,6′S,24a′S)-6′- {[(cyclopentyloxy)carbonyl]amino}-17′-fluoro- 19′,19′-dioxido-5′,21′,24′-trioxo-2-vinyl- 1′,2′,3′,5′,6′,7′,8′,9′,10′,11′,12′,13′,14′,20′,21′,23′, 24′,24a′-octadecahydrospiro[cyclopropane- 1,22′-pyrrolo[2,1-g] [1,2,5,8,18]benzothiatetraazacycloicosin]-2′-yl 4-fluoro-1,3-dihydro-2H-isoindole-2-carboxylate | M+ = 841.3 MS method D | | 5.960 HPLC method A |
| 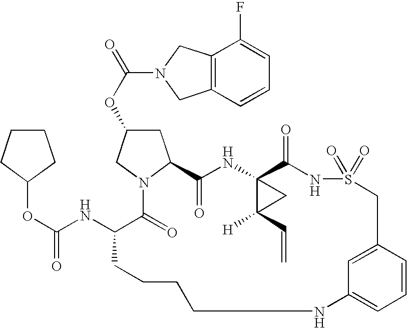 | Example 204: (1R,2S,18′S,22′R,23a′S)-18′- {[(cyclopentyloxy)carbonyl]amino}-6′,6′- dioxido-1′,4′,19′-trioxo-2-vinyl- 1′H,2′H,4′H,5′H,7′H,13′H,14′H,15′H,16′H,17′H, 18′H,19′H,21′H,22′H,23′H,23a′H- spiro[cyclopropane-1,3′- [6]thia[2,5,13,20]tetraaza[12,8](metheno)pyrrolo [2,1-g][1,2,5,8,15]thiatetraazacyclohenicosin]- 22′-yl 4-fluoro-1,3-dihydro-2H-isoindole- 2-carboxylate | M+ = 795.3 MS method D | | 4.33 HPLC method A |
| 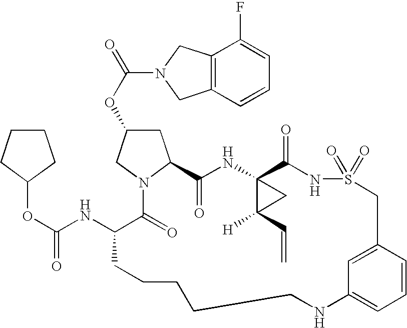 | Example 205: (1R,2S,19′S,23′R,24a′S)-19′- {[(cyclopentyloxy)carbonyl]amino}-6′,6′- dioxido-1′,4′,20′-trioxo-2-vinyl- 1′H,2′H,4′H,5′H,7′H,13′H,14′H,15′H,16′H,17′H, 18′H,19′H,20′H,22′H,23′H,24′H,24a′H- spiro[cyclopropane-1,3′- [6]thia[2,5,13,21]tetraaza[12,8](metheno)pyrrolo [2,1-g][1,2,5,8,16]thiatetraazacyclodocosin]-23′- yl 4-fluoro-1,3-dihydro-2H-isoindole-2- carboxylate | M+ = 809.2 MS method D | | 4.36 HPLC method A |
| 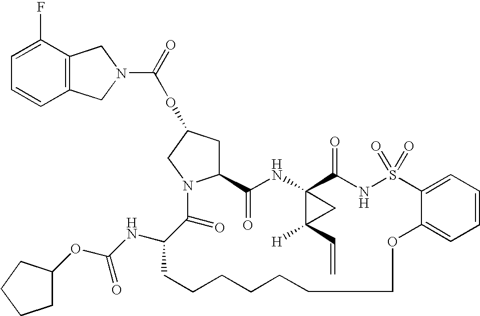 | Example 206: (1R,2S,2′R,6′S,24a′S)-6′- {[(cyclopentyloxy)carbonyl]amino}-19′,19′- dioxido-5′,21′,24′-trioxo-2-vinyl- 1′,2′,3′,6′,7′,8′,9′,10′,11′,12′,13′,20′,21′,23′,24′,24a′- hexadecahydro-5′H-spiro[cyclopropane- 1,22′-pyrrolo[2,1-g] [18,1,2,5,8]benzoxathiatriazacycloicosin]-2′-yl 4-fluoro-1,3-dihydro-2H-isoindole-2-carboxylate | M+ = 824.2 MS method D | | 5.376 HPLC method A |
| 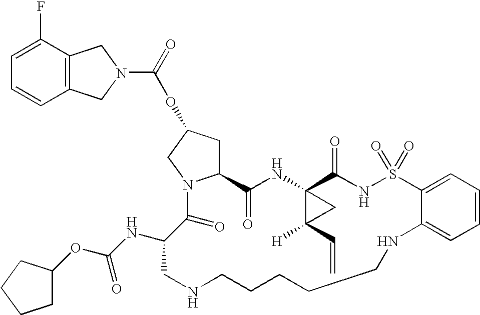 | Example 207: (1R,2S,2′R,6′S,24a′S)-6′- {[(cyclopentyloxy)carbonyl]amino}-19′,19′- dioxido-5′,21′,24′-trioxo-2-vinyl-1′,2′,3′,5′,6′, 7′,8′,9′,10′,11′,12′,13′,14′,20′,21′,23′,24′,24a′-octa- decahydrospiro[cyclopropane-1,22′-pyrrolo[2,1- g][1,2,5,8,12,18]benzothiapentaazacycloicosin]- 2′-yl 4-fluoro-1,3-dihydro-2H-isoindole-2- carboxylate | M+ = 824.2 MS method D | | HPLC method A |
| 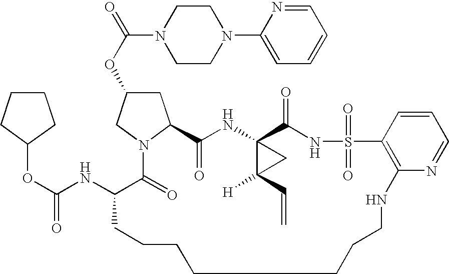 | Example 208: (1R,2S,13′S,17′R,18a′S)-13′- {[(cyclopentyloxy)carbonyl]amino}-24′,24′- dioxido-14′,19′,22′-trioxo-2-vinyl-5′,6′,7′,8′, 9′,10′,11′,12′,13′,14′,16′,17′,18′,18a′,19′,20′,22′,23′- octadecahydrospiro[cyclopropane-1,21′-pyrido [2,3-s]pyrrolo[2,1-g][1,2,5,8,18]thiatetraaza- cycloicosin]-17′-yl 4-pyridin-2-ylpiperazine-1- carboxylate | | 3.371; M − H = 848.3 LC MS method E | 3.345 HPLC method C |
| 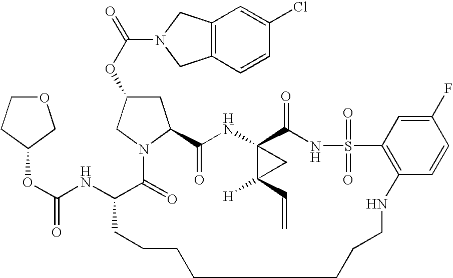 | Example 209: (1R,2S,2′R,6′S,24a′S)-17′-fluoro- 19′,19′-dioxido-5′,21′,24′-trioxo-6′-({[(3R)- tetrahydrofuran-3-yloxy]carbonyl}amino)-2- vinyl-1′,2′,3′,5′,6′,7′,8′,9′,10′,11′,12′,13′,14′,20′, 21′,23′,24′,24a′-octadecahydrospiro [cyclopropane-1,22′-pyrrolo[2,1- g][1,2,5,8,18]benzothiatetraazacycloicosin]-2′-yl 5-chloro-1,3-dihydro-2H-isoindole-2-carboxylate | | 4.366; M + H = 859.3 LC MS method E | 4.261 HPLC method C |
| 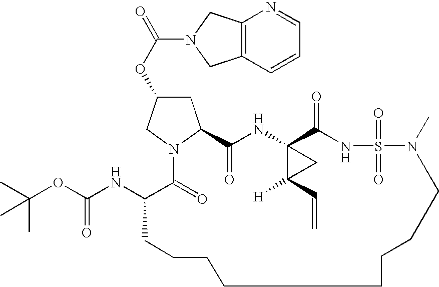 | Example 210: (1R,2S,16′S,20′R,21a′S)-16′-[(tert- butoxycarbonyl)amino]-7′-methyl-6′,6′-dioxido- 1′,4′,17′-trioxo-2-vinyloctadecahydro-7′H-spiro [cyclopropane-1,3′-pyrrolo[2,1-g][1,2,5,8,19] thiatetraazacyclononadecin]-20′-yl 5,7-dihydro- 6H-pyrrolo[3,4-b]pyridine-6-carboxylate | | 3.977; M + H = 746.3 LC MS method E | 3.623 HPLC method C |
| 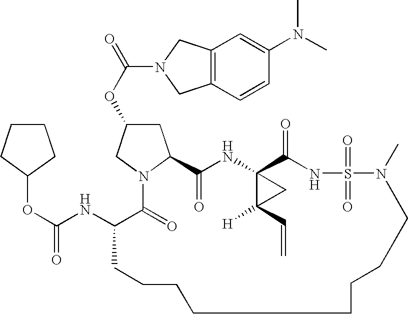 | Example 211: (1R,2S,16′S,20′R,21a′S)-16′- {[(cyclopentyloxy)carbonyl]amino}-7′-methyl- 6′,6-dioxido-1′,4′,17′-trioxo-2-vinyloctadeca- hydro-7′H-spiro[cyclopropane-1,3′-pyrrolo[2,1- g][1,2,5,8,19]thiatetraazacyclononadecin]-20′-yl 5-(dimethylamino)-1,3-dihydro-2H-isoindole-2- carboxylate | | 3.597; M + H = 800.2 LC MS method E | HPLC method C |
| 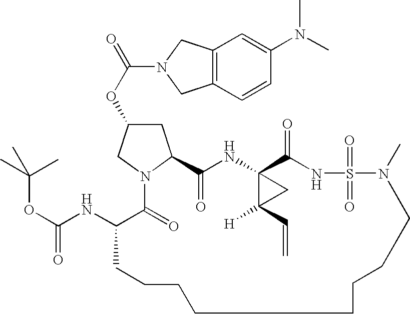 | Example 212: (1R,2S,16′S,20′R,21a′S)-16′-[(tert- butoxycarbonyl)amino]-7′-methyl-6′,6′-dioxido- 1′,4′,17′-trioxo-2-vinyloctadecahydro-7′H- spiro[cyclopropane-1,3′-pyrrolo[2,1-g] [1,2,5,8,19]thiatetraazacyclononadecin]-20′-yl 5- (dimethylamino)-1,3-dihydro-2H-isoindole-2- carboxylate | | 3.645; M + H = 788.2 LC MS method E | |
| 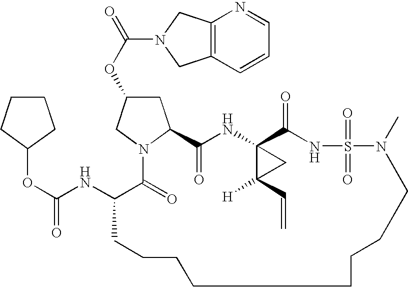 | Example 213: (1R,2S,16′S,20′R,21a′S)-16′- {[(cyclopentyloxy)carbonyl]amino}-7′-methyl- 6′,6′-dioxido-1′,4′,17′-trioxo-2-vinyloctadeca- hydro-7′H-spiro[cyclopropane-1,3′-pyrrolo[2,1- g][1,2,5,8,19]thiatetraazacyclononadecin]-20′-yl 5,7-dihydro-6H-pyrrolo[3,4-b]pyridine-6- carboxylate | | 3.909; M + H = 758.2 LC MS method E | 3.714 HPLC method C |
| 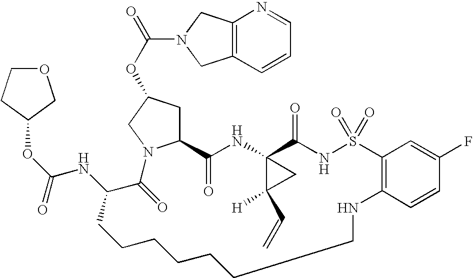 | Example 214: (1R,2S,2′R,6′S,24a′S)-17′-fluoro- 19′,19′-dioxido-5′,21′,24′-trioxo-6′-({[(3R)- tetrahydrofuran-3-yloxy]carbonyl}amino)-2- vinyl-1′,2′,3′,5′,6′,7′,8′,9′,10′,11′,12′,13′,14′,20′, 21′,23′,24′,24a′-octadecahydrospiro [cyclopropane-1,22′-pyrrolo[2,1- g][1,2,5,8,18]benzothiatetraazacycloicosin]-2′-yl 5,7-dihydro-6H-pyrrolo[3,4-b]pyridine-6- carboxylate | M+ = 826.2 MS method D | | 4.74 HPLC method A |
| 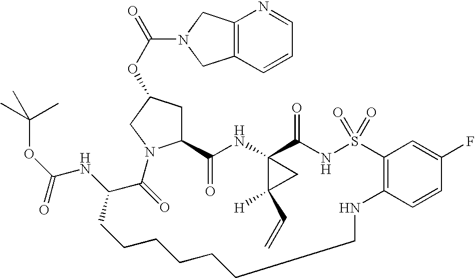 | Example 215: (1R,2S,2′R,6′S,24a′S)-6′-[(tert- butoxycarbonyl)amino]-17′-fluoro-19′,19′- dioxido-5′,21′,24′-trioxo-2-vinyl- 1′,2′,3′,5′,6′,7′,8′,9′,10′,11′,12′,13′,14′,20′,21′,23′, 24′,24a′-octadecahydrospiro[cyclopropane- 1,22′-pyrrolo[2,1-g] [1,2,5,8,18]benzothiatetraazacycloicosin]-2′-yl 5,7-dihydro-6H-pyrrolo[3,4-b]pyridine-6- carboxylate | M+ = 812.2 MS method D | | 5.21 HPLC method A |
| 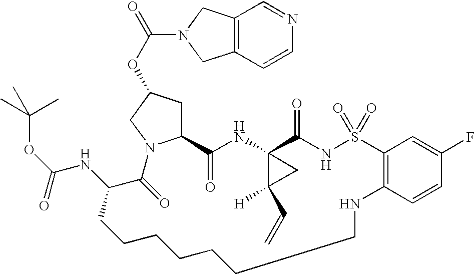 | Example 216: (1R,2S,2′R,6′S,24a′S)-6′-[(tert- butoxycarbonyl)amino]-17′-fluoro-19′,19′- dioxido-5′,21′,24′-trioxo-2-vinyl- 1′,2′,3′,5′,6′,7′,8′,9′,10′,11′,12′,13′,14′,20′,21′,23′, 24′,24a′-octadecahydrospiro[cyclopropane- 1,22′-pyrrolo[2,1-g] [1,2,5,8,18]benzothiatetraazacycloicosin]-2′-yl 1,3-dihydro-2H-pyrrolo[3,4-c]pyridine-2- carboxylate | M+ = 812.2 MS method D | | 4.923 HPLC method A |
| 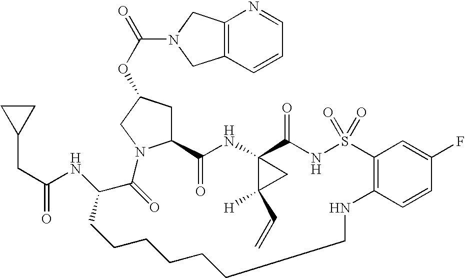 | Example 217: (1R,2S,2′R,6′S,24a′S)-6′- [(cyclopropylacetyl)amino]-17′-fluoro-19′,19′- dioxido-5′,21′,24′-trioxo-2-vinyl- 1′,2′,3′,5′,6′,7′,8′,9′,10′,11′,12′,13′,14′,20′,21′,23′, 24′,24a′-octadecahydrospiro[cyclopropane- 1,22′-pyrrolo[2,1-g] [1,2,5,8,18]benzothiatetraazacycloicosin]-2′-yl 5,7-dihydro-6H-pyrrolo[3,4-b]pyridine-6- carboxylate | M+ = 794.2 MS method D | | 4.858 HPLC method A |
| 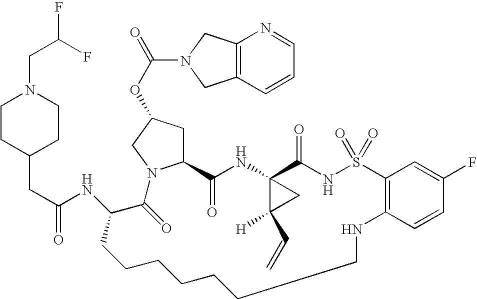 | Example 218: (1R,2S,2′R,6′S,24a′S)-6′-({[1-(2,2- difluoroethyl)piperidin-4-yl]acetyl}amino)-17′- fluoro-19′,19′-dioxido-5′,21′,24′-trioxo-2-vinyl- 1′,2′,3′,5′,6′,7′,8′,9′,10′,11′,12′,13′,14′,20′,21′,23′, 24′,24a′-octadecahydrospiro[cyclopropane- 1,22′-pyrrolo[2,1-g] [1,2,5,8,18]benzothiatetraazacycloicosin]-2′-yl 5,7-dihydro-6H-pyrrolo[3,4-b]pyridine-6- carboxylate | M+ = 901.2 MS method D | | 4.353 HPLC method A |
| 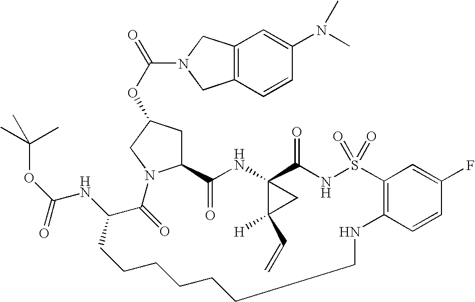 | Example 219: (1R,2S,2′R,6′S,24a′S)-6′-[(tert- butoxycarbonyl)amino]-17′-fluoro-19′,19′- dioxido-5′,21′,24′-trioxo-2-vinyl- 1′,2′,3′,5′,6′,7′,8′,9′,10′,11′,12′,13′,14′,20′,21′,23′, 24′,24a′-octadecahydrospiro[cyclopropane- 1,22′-pyrrolo[2,1-g] [1,2,5,8,18]benzothiatetraazacycloicosin]-2′-yl 5-(dimethylamino)-1,3-dihydro-2H-isoindole-2- carboxylate | M+ = 854.2 MS method D | | 3.20 HPLC method A |
| 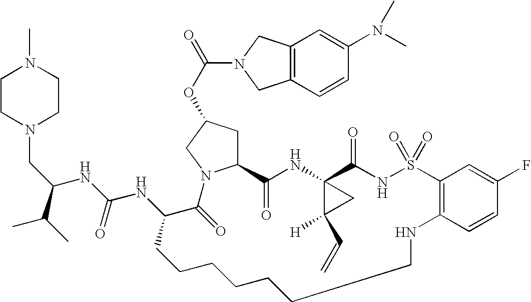 | Example 220: (1R,2S,2′R,6′S,24a′S)-17′-fluoro- 6′-[({(1S)-2-methyl-1-[(4-methylpiperazin-1- yl)methyl]propyl}carbamoyl)amino]-19′,19′- dioxido-5′,21′,24′-trioxo-2-vinyl- 1′,2′,3′,5′,6′,7′,8′,9′,10′,11′,12′,13′,14′,20′,21′,23′, 24′,24a′-octadecahydrospiro[cyclopropane- 1,22′-pyrrolo[2,1-g] [1,2,5,8,18]benzothiatetraazacycloicosin]-2′-yl 5-(dimethylamino)-1,3-dihydro-2H-isoindole-2- carboxylate | M+ = 965.4 MS method D | | 2.25 HPLC method A |
| 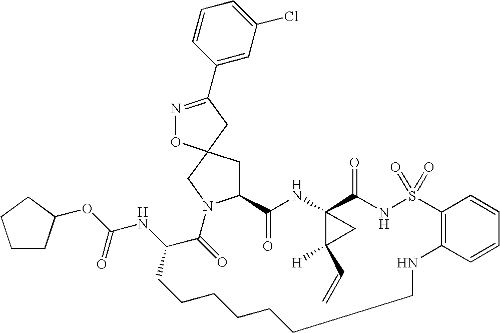 | Example 221: Cyclopentyl [(1R,2S,2′R,6′S,24a′S)- 2′-spiro-[3-(3-chloro-phenyl)-4,5-dihydro- isoxazole]-19′,19′-dioxido-5′,21′,24′-trioxo-2-vinyl- 1′,2′,3′,5′,6′,7′,8′,9′,10′,11′,12′,13′,14′,20′,21′,23′,24′, 24a′-octadecahydrospiro[cyclopropane-1,22′- pyrrolo[2,1-g][1,2,5,8,18] benzothiatetraazacycloicosin]-6′-yl]carbamate | M + 1 = 809 MS method D | | 6.22 HPLC method A |
| 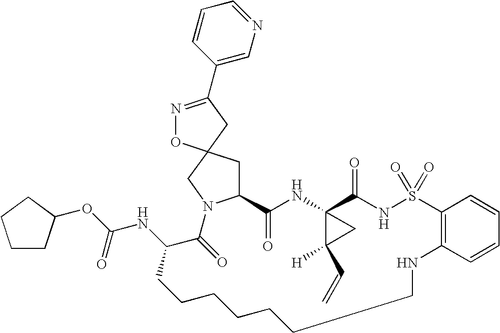 | Example 222: Cyclopentyl [(1R,2S,2′R,6′S,24a′S)- 2′-spiro-[3-(4,5-dihydro-isoxazol-3-yl)-pyridine]- 19′,19′-dioxido-5′,21′,24′-trioxo-2-vinyl- 1′,2′,3′,5′,6′,7′,8′,9′,10′,11′,12′,13′,14′,20′,21′,23′,24′, 24a′-octadecahydrospiro[cyclopropane- 1,22′-pyrrolo[2,1-g] [1,2,5,8,18]benzothiatetraazacycloicosin]-6′- yl]carbamate | M − 1 = 774 MS method D | | 5.01 HPLC method A |
| 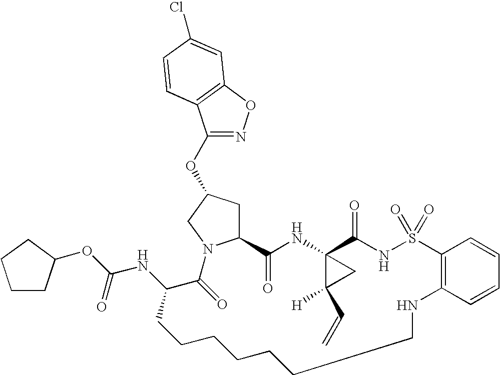 | Example 223: Cyclopentyl [(1R,2S,2′R,6′S,24a′S)- 2′-[6-chloro-benzo[d]isoxazol-3-yloxy]-19′,19′- dioxido-5′,21′,24′-trioxo-2-vinyl- 1′,2′,3′,5′,6′,7′,8′,9′,10′,11′,12′,13′,14′,20′,21′,23′,24′, 24a′-octadecahydrospiro[cyclopropane- 1,22′-pyrrolo[2,1-g] [1,2,5,8,18]benzothiatetraazacycloicosin]-6′- yl]carbamate | M + 1 = 811 MS method D | | 6.22 HPLC method A |
| 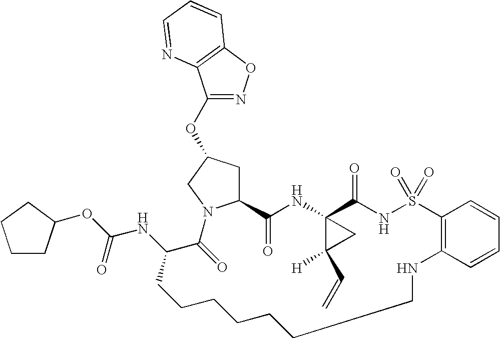 | Example 224: Cyclopentyl [(1R,2S,2′R,6′S,24a′S)- 2′-[isoxazolo[4,5-b]pyridin-3-yloxy]-19′,19′- dioxido-5′,21′,24′-trioxo-2-vinyl- 1′,2′,3′,5′,6′,7′,8′,9′,10′,11′,12′,13′,14′,20′,21′,23′,24′, 24a′-octadecahydrospiro[cyclopropane- 1,22′-pyrrolo[2,1-g] [1,2,5,8,18]benzothiatetraazacycloicosin]-6′- yl]carbamate | M + 1 = 778 MS method D | | 5.69 HPLC method A |
| 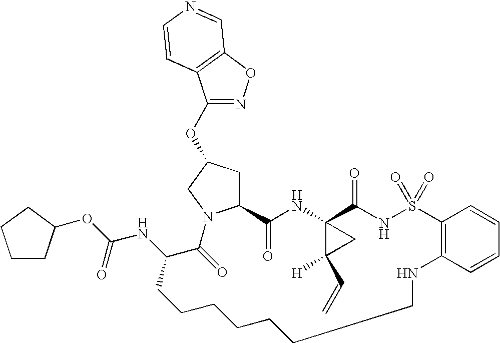 | Example 225: Cyclopentyl [(1R,2S,2′R,6′S,24a′S)- 2′-[isoxazolo[5,4-c]pyridin-3-yloxy]-19′,19′- dioxido-5′,21′,24′-trioxo-2-vinyl- 1′,2′,3′,5′,6′,7′,8′,9′,10′,11′,12′,13′,14′,20′,21′,23′,24′, 24a′-octadecahydrospiro[cyclopropane- 1,22′-pyrrolo[2,1-g] [1,2,5,8,18]benzothiatetraazacycloicosin]-6′- yl]carbamate | M + 1 = 778 MS method D | | 5.59 HPLC method A |

### Example 226

### 4-Fluoro-1,3-dihydro-isoindole-2-carboxylic acid (2R,5S,18aS)-16-cyclobutylmethyl-5-cyclopentyloxycarbonylamino-4,14,15,18-tetraoxo-octadecahydro-3a,13,17-triaza-cyclopentacycloheptadecen-2-yl ester

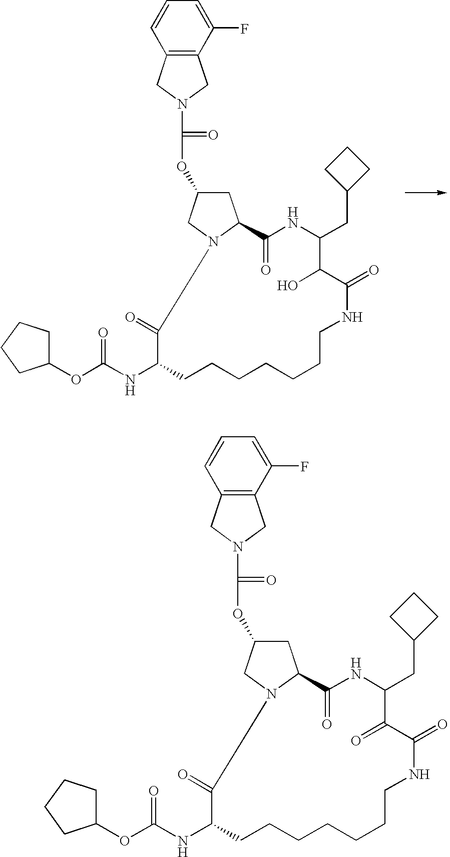

A solution of 0.01 g (0.014 mmol) of 4-fluoro-1,3-dihydro-isoindole-2-carboxylic acid (2R,5S,18aS)-16-cyclobutylmethyl-5-cyclopentyloxycarbonylamino-15-hydroxy-4,14,18-trioxo-octadecahydro-3a,13,17-triaza-cyclopentacycloheptadecen-2-yl ester in 0.1 mL of DMSO is treated with 0.012 g (0.042 mmol) of IBX for 3 hours and chromatographed by RP-HPLC (method G) to give the title compound; MS (method D): 712 [M+1]; HPLC (method A) t_{R} (min) 5.24

### Preparation of 4-fluoro-1,3-dihydro-isoindole-2-carboxylic acid (2R,5S,18aS)-16-cyclobutylmethyl-5-cyclopentyloxycarbonylamino-15-hydroxy-4,14,18-trioxo-octadecahydro-3a,13,17-triaza-cyclopentacycloheptadecen-2-yl ester

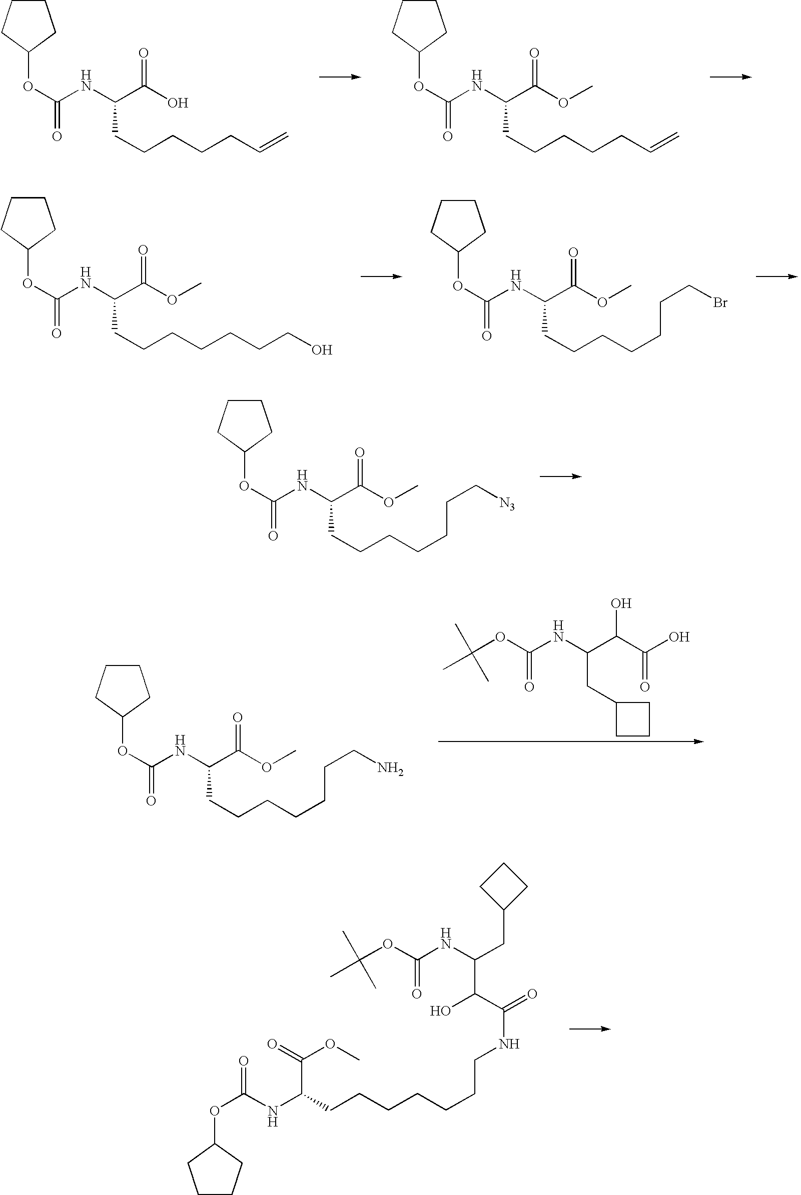

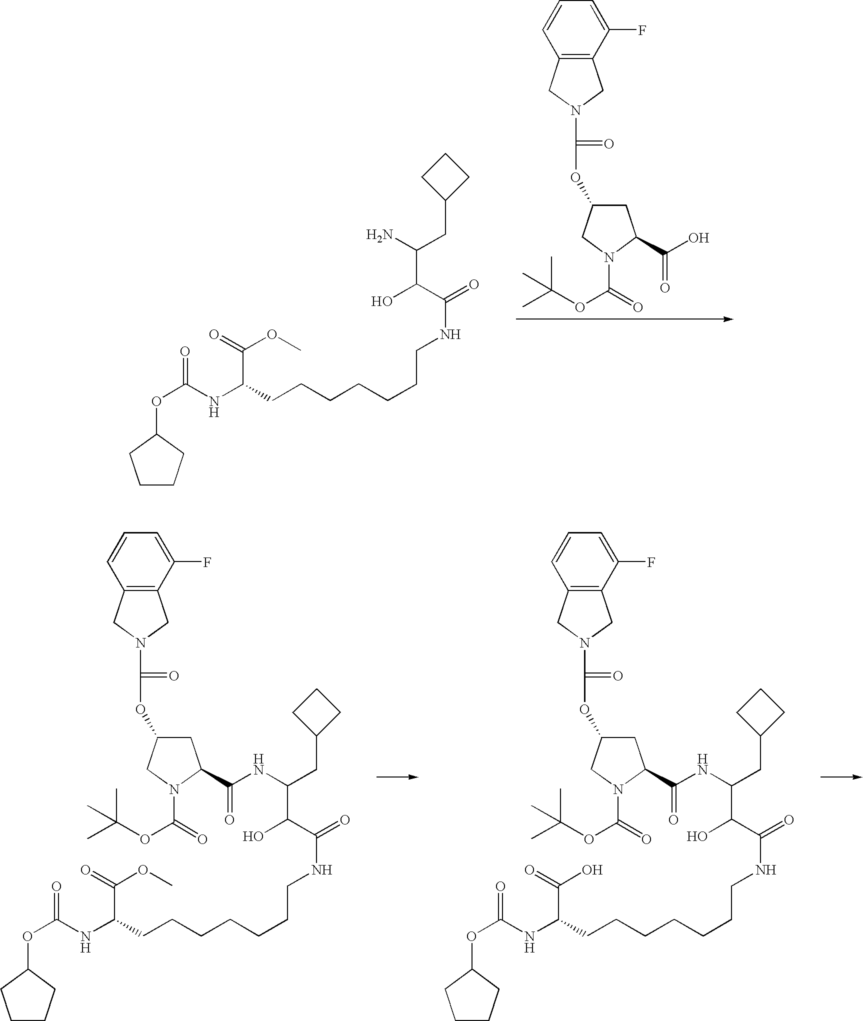

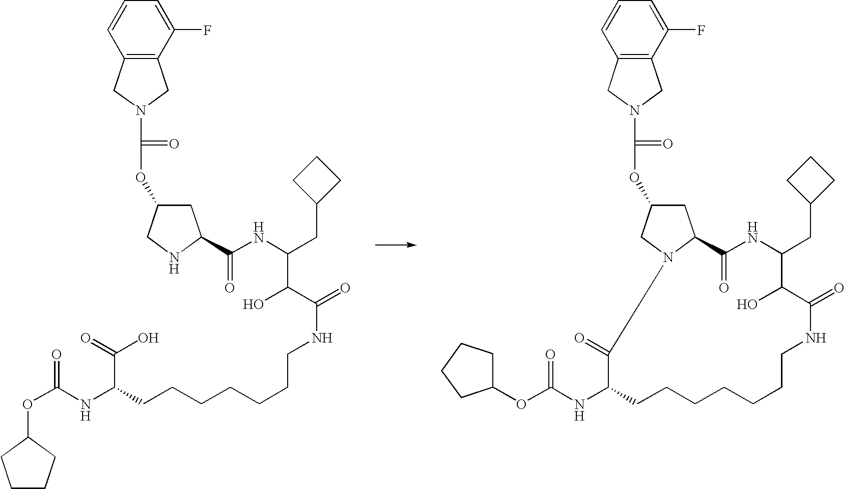

### Step 1

### (S)-2-Cyclopentyloxycarbonylamino-non-8-enoic acid methyl ester

A solution of 18.1 g (63.87 mmol) of (S)-2-cyclopentyloxycarbonylamino-non-8-enoic acid in 300 mL of acetone is treated with 10.232 g (102.2 mmol) of KHCO₃ and 22.666 g (159.69 mmol) of iodomethane and then heated up to reflux. Upon completion the reaction mixture is cooled down, salts are filtered-off and the filtrate is concentrated, taken up in EtOAc, washed with saturated aqueous NaHCO₃ and brine. The organics are dried over Na₂SO₄, concentrated in vacuo to give the title compound; MS (method D): 298 [M+1]

### Step 2

### (S)-2-Cyclopentyloxycarbonylamino-9-hydroxy-nonanoic acid methyl ester

A solution of 25.65 g (86.25 mmol) of (S)-2-cyclopentyloxycarbonylamino-non-8-enoic acid methyl ester in 400 mL of absolute THF is cooled to 0° C. and treated by drop wise addition of 275 mL (120.7 mmol) of 9-BBN (0.5M in THF solution) while maintaining temperature below 5° C. The reaction mixture is stirred at RT under completion, cooled to 0° C., treated by drop wise addition of 80 mL of a 5% NaHCO₃ aqueous solution, then by careful addition of 16.3 mL of 35% H₂O₂ in water while maintaining the temperature below 12° C. The reaction mixture is stirred at RT for 1.5 hour, treated with 100 mL of saturated aqueous NaHCO₃ and 100 mL water. The organics are washed with brine and water, combined, dried (Na₂SO₄), concentrated and chromatographed on silica gel (eluent Hexane/EtOAc 1:1) to give the title compound; MS (method D): 316 [M+1]

### Step 3

### (S)-9-Bromo-2-cyclopentyloxycarbonylamino-nonanoic acid methyl ester

A solution of 5.5 g (17.44 mmol) of (S)-2-cyclopentyloxycarbonylamino-9-hydroxy-nonanoic acid methyl ester in 60 mL of CH₂Cl₂ is treated with 4.851 g (18.31 mmol) of triphenylphosphine and 3.36 g (18.31 mmol) of N-bromosuccinimide and stirred overnight at RT. The crude reaction mixture is chromatographed on silica gel (eluent Hexane/EtOAc 7:2) to give the title compound; MS (method D): 378 [M+1]

### Step 4

### (S)-9-Azido-2-cyclopentyloxycarbonylamino-nonanoic acid methyl ester

A solution of 1.8 g (4.76 mmol) of (S)-9-bromo-2-cyclopentyloxycarbonylamino-nonanoic acid methyl ester in 20 mL DMF is treated with 1.25 g (19.03 mmol) of sodium azide and stirred at 50° C. for 2 hours. The reaction mixture is quenched with saturated aqueous NaHCO3 and extracted with ethylether. The organics are washed with brine, dried over Na₂SO₄ and concentrated to give the title compound; MS (method D): 341 [M+1]

### Step 5

### (S)-9-Amino-2-cyclopentyloxycarbonylamino-nonanoic acid methyl ester

A solution of 1.41 g (4.14 mmol) of (S)-9-azido-2-cyclopentyloxycarbonylamino-nonanoic acid methyl ester in 50 mL ethanol is hydrogenated over Pd/Carbon (0.2 g, 10%) at RT under H₂ atmosphere. The reaction mixture is filtered through Celite and the filtrate concentrated to give the title compound; MS (method D): 315 [M+1]

### Step 6

### (S)-9-(3-tert-Butoxycarbonylamino-4-cyclobutyl-2-hydroxy-butyrylamino)-2-cyclopentyloxycarbonylamino-nonanoic acid methyl ester

A solution of 0.4 g (1.27 mmol) of (S)-9-amino-2-cyclopentyloxycarbonylamino-nonanoic acid methyl ester and 0.417 g (1.52 mmol) of 3-tert-butoxycarbonylamino-4-cyclobutyl-2-hydroxy-butyric acid in 10 mL CH₂Cl₂ is treated with 0.212 g (1.53 mmol) of 1-hydroxy-7-azabenzotriazole and 0.443 g (2.29 mmol) of N-(3-dimethylaminopropyl)-N′-ethyl-carbodiimide hydrochloride, followed by 0.217 mL (1.53 mmol) of triethylamine. The reaction mixture is stirred overnight at RT and chromatographed on silica gel (eluent Hexane/EtOAc 3:2) to give the title compound; MS (method D): 570 [M+1]

### Step 7

### (S)-9-(3-Amino-4-cyclobutyl-2-hydroxy-butyrylamino)-2-cyclopentyloxycarbonylamino-nonanoic acid methyl ester

A solution of 0.358 g (0.63 mmol) of (S)-9-(3-tert-butoxycarbonylamino-4-cyclobutyl-2-hydroxy-butyrylamino)-2-cyclopentyloxycarbonylamino-nonanoic acid methyl ester in 1.57 mL of 4N HCl in dioxane is stirred at RT. Upon completion the reaction mixture is concentrated in vacuo to give the title compound; MS (method D): 470 [M+1]

### Step 8

### 4-Fluoro-1,3-dihydro-isoindole-2-carboxylic acid (3R,5S)-1-tert-butoxycarbonyl-5-[1-cyclobutylmethyl-2-((S)-8-cyclopentyloxycarbonylamino-8-methoxycarbonyl-octylcarbamoyl)-2-hydroxy-ethylcarbamoyl]-pyrrolidin-3-yl ester

A solution of 0.306 g (0.65 mmol) of (S)-9-(3-amino-4-cyclobutyl-2-hydroxy-butyrylamino)-2-cyclopentyloxycarbonylamino-nonanoic acid methyl ester and 0.283 g (0.72 mmol) of (2S,4R)-4-(4-fluoro-1,3-dihydro-isoindole-2-carbonyloxy)-pyrrolidine-1,2-dicarboxylic acid 1-tert-butyl ester 15 mL CH₂Cl₂ is treated with 0.109 g (0.78 mmol) of 1-hydroxy-7-azabenzotriazole and 0.139 mL (0.98 mmol) of triethylamine, followed by 0.227 g (1.17 mmol) of N-(3-dimethylaminopropyl)-N′-ethyl-carbodiimide hydrochloride. The reaction mixture is stirred overnight at RT and chromatographed by RP-HPLC (method G) to give the title compound; MS (method D): 846 [M+1

### Step 9

### 4-Fluoro-1,3-dihydro-isoindole-2-carboxylic acid (3R,5S)-1-tert-butoxycarbonyl-5-[2-((S)-8-carboxy-8-cyclopentyloxycarbonylamino-octylcarbamoyl)-1-cyclobutylmethyl-2-hydroxy-ethylcarbamoyl]-pyrrolidin-3-yl ester

A suspension of 0.353 g (0.42 mmol) of 4-fluoro-1,3-dihydro-isoindole-2-carboxylic acid (3R,5S)-1-tert-butoxycarbonyl-5-[1-cyclobutylmethyl-2-((S)-8-cyclopentyloxycarbonylamino-8-methoxycarbonyl-octylcarbamoyl)-2-hydroxy-ethylcarbamoyl]-pyrrolidin-3-yl ester in 5 mL methanol and 5 mL water is treated with 0.204 g (8.34 mmol) of LiOH and stirred overnight at RT. Methanol is removed in vacuo, the resulting aqueous phase is acidified to pH 6 with 2N HCl and extracted with CH₂Cl₂. The organics are dried over Na₂SO₄ to give the title compound; MS (method D): 832 [M+1]

### Step 10

### 4-Fluoro-1,3-dihydro-isoindole-2-carboxylic acid (3R,5S)-5-[2-((S)-8-carboxy-8-cyclopentyloxycarbonylamino-octylcarbamoyl)-1-cyclobutylmethyl-2-hydroxy-ethylcarbamoyl]-pyrrolidin-3-yl ester

The title compound is obtained from 0.302 g (0.254 mmol) of 4-fluoro-1,3-dihydro-isoindole-2-carboxylic acid (3R,5S)-1-tert-butoxycarbonyl-5-[2-((S)-8-carboxy-8-cyclopentyloxycarbonylamino-octylcarbamoyl)-1-cyclobutylmethyl-2-hydroxy-ethylcarbamoyl]-pyrrolidin-3-yl ester according to the procedure described in step 7; MS (method D): 732 [M+1]

### Step 11

### 4-Fluoro-1,3-dihydro-isoindole-2-carboxylic acid (2R,5S,18aS)-16-cyclobutylmethyl-5-cyclopentyloxycarbonylamino-15-hydroxy-4,14,18-trioxo-octadecahydro-3a,13,17-triaza-cyclopentacycloheptadecen-2-yl ester

A solution of 0.293 g (0.28 mmol) of 4-fluoro-1,3-dihydro-isoindole-2-carboxylic acid (3R,5S)-5-[2-((S)-8-carboxy-8-cyclopentyloxycarbonylamino-octylcarbamoyl)-1-cyclobutylmethyl-2-hydroxy-ethylcarbamoyl]-pyrrolidin-3-yl ester in 30 mL of CH₂Cl₂ is treated with 0.479 mL (2.8 mmol) of Hunig's base, followed by 0.532 g (1.4 mmol) of HATU. The reaction mixture is stirred at RT under completion and chromatographed by RP-HPLC (method G) to give the title compound; MS (method D): 714 [M+1]

### Synthesis of intermediates

### Preparation of 5-chloroisoindoline

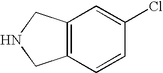

Prepared as described by T.-Y-Tsai in Bioorg. Med. Chem. Lett. 2006, 16, 3268 starting from 5-chloro-1H-isoindole-1,3(2H)-dione.

### Preparation of 5-morpholin-4-ylisoindoline

### Step 1

### 5-bromoisoindoline

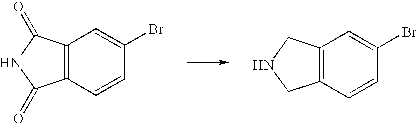

To a mixture of 4.5 g (20 mmol) 5-bromo-1H-isoindole-1,3(2H)-dione in 10 mL THF is added 81 mL Borane-THF complex (1 M) and the mixture is refluxed overnight. After cooling to rt 150 mL MeOH and 80 mL 6 N aq. HCl are carefully added and the mixture is refluxed for 1 h. The mixture is concentrated under reduced pressure, water and DCM are added and the aq. layer is extracted with DCM (2×) and ether (2×). The pH of the aq. layer is adjusted to 11 using conc. Aq. NaOH and extracted with DCM (4×). The combined organic layers of this last extraction are dried over Na2SO4, concentrated in vacuo and the residue is used without further purification.

LC MS (method E) t_{R}=0.346 min, M+H=200.1

### Step 2

### tert-butyl 5-bromo-1,3-dihydro-2H-isoindole-2-carboxylate

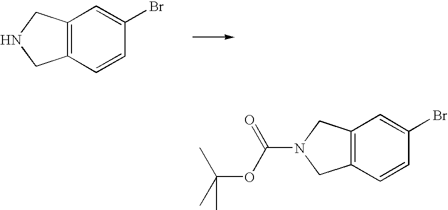

To a mixture of 2.2 g (11 mmol) 5-bromoisoindoline in 90 mL DCM is added at 0° C. a solution of 2.9 g (13 mmol) Boc2O in 20 mL DCM followed by 3.0 mL (20 mmol) TMEDA. The mixture is stirred at 5° C. overnight and 250 mL 2 N aq. HCl is added and the mixture is stirred for additional 20 min at 5° C. The aq. layer is extracted with DCM and the combined organic layers are dried over Na2SO4 and concentrated under reduced pressure. The residue is purified by FC on silica.

HPLC (method C) t_{R}=4.141 min

### Step 3

### tert-butyl 5-morpholin-4-yl-1,3-dihydro-2H-isoindole-2-carboxylate

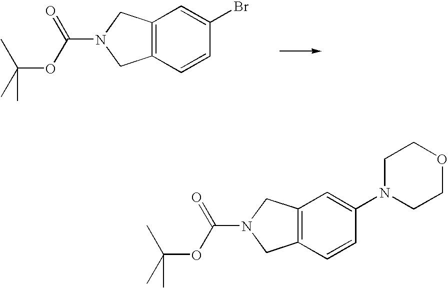

A mixture of 600 mg (2.0 mmol) tert-butyl 5-bromo-1,3-dihydro-2H-isoindole-2-carboxylate, 0.2 mL (2.4 mmol) morpholine, 268 mg (2.8 mmol) sodium tert.butoxide, 18 mg (0.02 mmol) Pd2(dba)3 and 37 mg (0.06 mmol) rac-BINAP in 4 mL toluene is stirred at 80 0° C. for 3 h. The mixture is cooled to rt, ethyl ether is added and the precipitate is filtered off and dried.

LC MS (method E) t_{R}=3.592 min, M+H=305.2

HPLC (method C) t_{R}=2.870 min

### Step 4

### 5-morpholin-4-ylisoindoline(hydrochloride)

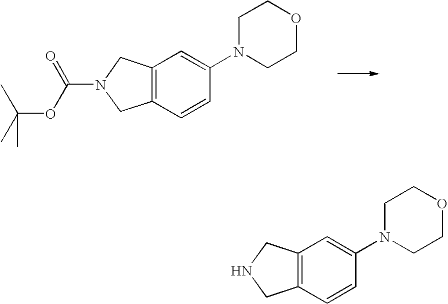

A mixture of 130 mg (0.4 mmol) tert-butyl 5-morpholin-4-yl-1,3-dihydro-2H-isoindole-2-carboxylate, 4 mL 4 M HCl in dioxane and 4 mL dioxane is stirred for 3.5 h at rt. The mixture is concentrated and the crude is used without further purification.

LC MS (method E) t_{R}=0.264 min, M+H=205.1

The following isoindoline can be prepared as described above:

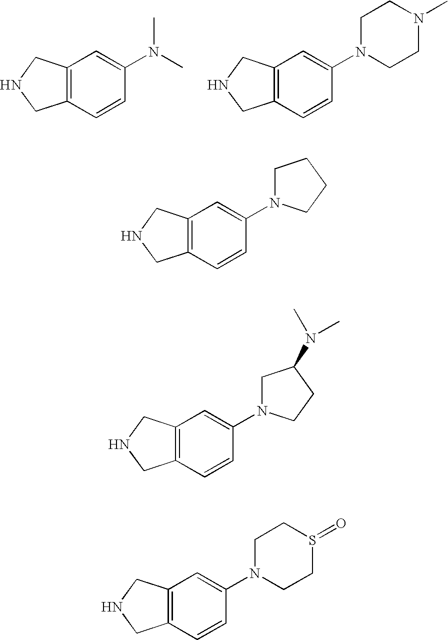

### Preparation of 1-(2,3-Dihydro-1H-isoindol-5-yl)cyclopropanamine

### Step 1

### 5-Cyano-1,3-dihydro-isoindole-2-carboxylic acid tert-butyl ester

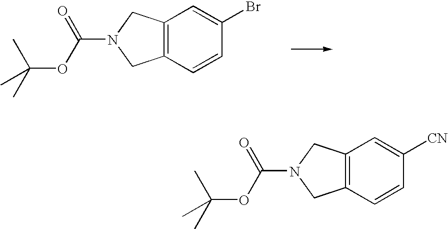

A mixture of 0.5 g (1.5 mmol) tert-butyl 5-bromo-1,3-dihydro-2H-isoindole-2-carboxylate, 626 mg (2.0 mmol) Zinc cyanide and 367 mg (0.2 mmol) Pd(PPh3)4 in 15 mL DMF is heated to 80° C. for 2 h. The mixture is partitioned between water and EtOAc and the aq. layer is extracted with EtOAc. The combined organic layers are washed with brine, dried and concentrated under reduced pressure to give a crude product which is purified by FC (silica gel).

LC MS (method E) t_{R}=4.161 min, M+H=244.9

### Step 2

### 5-(1-Amino-cyclopropyl)-1,3-dihydro-isoindole-2-carboxylic acid tert-butyl ester

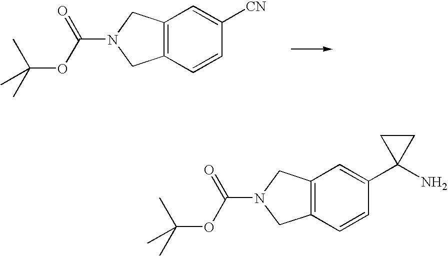

To a mixture of 250 mg (1.1 mmol) 5-Cyano-1,3-dihydro-isoindole-2-carboxylic acid tert-butyl ester and 0.3 mL (1.2 mmol) titanium-(VI)-isopropoxide in 5 mL ether is added 0.8 mL (2.3 mmol) ethylmagnesium bromide (3 M in ether) at −70° C. After 5 min the mixture is allowed to reach rt over 1 h and 0.3 mL (2.1 mmol) BF3*Et20 is added. After 1 h the mixture is quenched with 1N HCl and ether and a basic pH is adjusted using NaOH solution. The aq. layer is extracted with ether and the combined organic layers are dried and concentrated under reduced pressure. The crude product is purified by FC (silica gel)

### Step 3

### 1-(2,3-Dihydro-1H-isoindol-5-yl)cyclopropanamine(dihydrochloride)

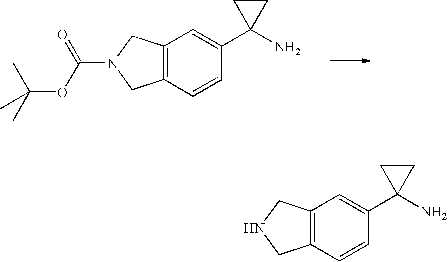

95 mg (0.3 mmol) of the 5-(1-Amino-cyclopropyl)-1,3-dihydro-isoindole-2-carboxylic acid tert-butyl ester is dissolved in 2 mL dioxane and 2 mL 4M HCl in dioxane are added. The mixture is stirred at rt for 3 h and concentrated in vacuo to yield the product which is used in the next step without further purification.

### Preparation of N-methyl-1,2,3,4-tetrahydroisoquinolin-5-amine

### Step 1

### 5-Amino-3,4-dihydro-1H-isoquinoline-2-carboxylic acid tert-butyl ester

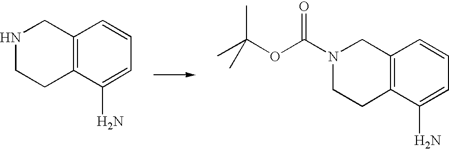

To a mixture of 5 g (34 mmol) 1,2,3,4-tetrahydro-5-aminoisoquinoline in 150 mL dioxane are added 11 mL aq. NaOH (3M) and 7.4 g (34 mmol) Boc2O at 0° C. The mixture is stirred at rt overnight, ice water is added and the mixture is extracted with EtOAc. The combined organic layers are washed with sat. NaHCO3-solution and brine, dried and concentrated in vacuo. The crude product is used in the next step without further purification.

LC MS (method E) t_{R}=2.636 min, M-Boc+H=149.2

### Step 2

### 5-Methylamino-3,4-dihydro-1H-isoquinoline-2-carboxylic acid tert-butyl ester and 5-Dimethylamino-3,4-dihydro-1H-isoquinoline-2-carboxylic acid tert-butyl ester

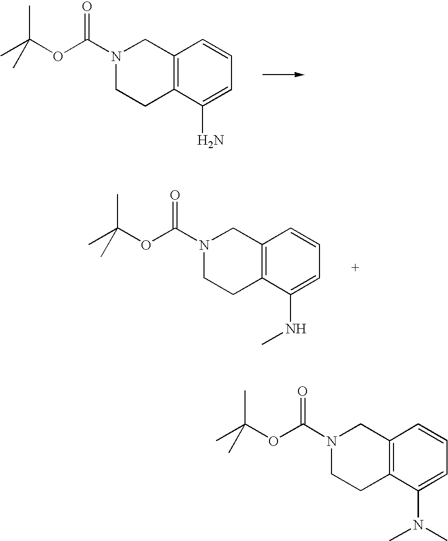

To a mixture of 8.2 g (33 mmol) 5-Amino-3,4-dihydro-1H-isoquinoline-2-carboxylic acid tert-butyl ester in 200 mL THF is added 3.3 g (83 mmol) NaH (60% in mineral oil) at 0° C. After 15 min 6.2 mL (99 mmol) methyliodid is added and the mixture is stirred at rt for 48 h. The mixture is poured on ice water and extracted with EtOAc. The combined organic layer is dried and concentrated to give a mixture of mono- and dimethylated product. The crude product is triturated with MeOH and the unsoluble solid is filtered off to give the pure monomethylated product. The filtrate is concentrated to give a mixture of mono- and dimethylated product.

LC MS (method E) t_{R}=2.076 min, M+H=277.1 (dimethyl)

LC MS (method E) t_{R}=3.261 min, M-Boc+H=263.2 (monomethyl)

### Step 3

### Methyl-(1,2,3,4-tetrahydro-isoquinolin-5-yl)-amine(dihydrochloride salt)

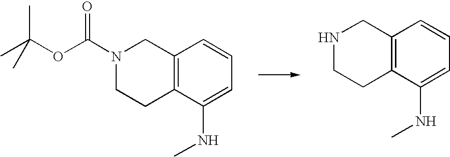

300 mg (1.2 mmol) of the pure 5-Methylamino-3,4-dihydro-1H-isoquinoline-2-carboxylic acid tert-butyl ester obtained in step 2 is dissolved in 5 mL dioxane and 5 mL 4M HCl in dioxane are added. The mixture is stirred at rt for 3 h and concentrated in vacuo to yield the product which is used in the next step without further purification.

LC MS (method E) t_{R}=0.256 min, M+H=163.1

### Preparation of N,N-dimethyl-1,2,3,4-tetrahydroisoquinolin-5-amine(dihydrochloride)

### Step 1

### Dimethylamino-3,4-dihydro-1H-isoquinoline-2-carboxylic acid tert-butyl ester

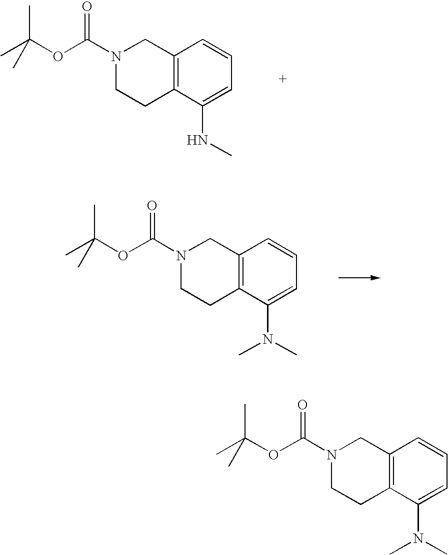

1 g of the mixture of mono- and dimethyl product of step 2 of the previous example is dissolved in 10 mL THF and 190 mg NaH (60% in mineral oil) is added at 0° C. After 15 min 0.35 mL methyliodide is added and the mixture is stirred at rt overnight. The mixture is poured on ice water and extracted with EtOAc. The combined organic layer is dried and concentrated to give the dimethylated product.

LC MS (method E) t_{R}=2.076 min, M+H=277.1

### Step 2

### N,N-dimethyl-1,2,3,4-tetrahydroisoquinolin-5-amine(dihydrochloride)

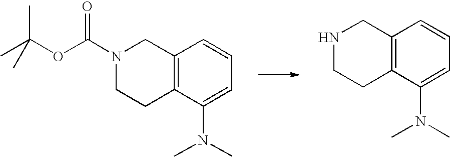

1.3 g (4.7 mmol) of the pure Dimethylamino-3,4-dihydro-1H-isoquinoline-2-carboxylic acid tert-butyl ester obtained in step 1 is dissolved in 15 mL dioxane and 15 mL 4M HCl in dioxane are added. The mixture is stirred at rt overnight and concentrated in vacuo to yield the product which is used in the next step without further purification.

LC MS (method E) t_{R}=0.349 min, M+H=177.3

### Preparation of 2-(4-methyl-piperazin-1-yl)-6,7-dihydro-5H-pyrrolo[3,4-d]pyrimidine

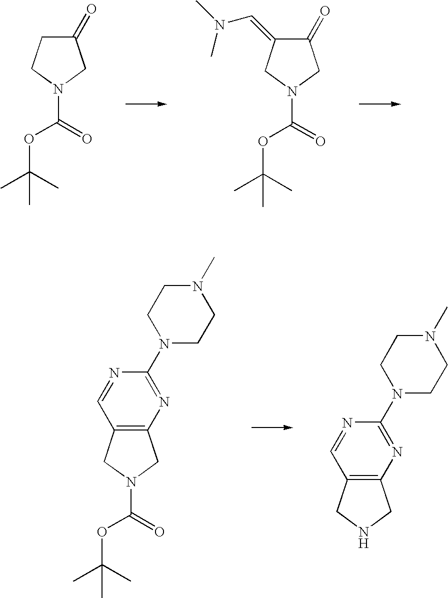

### Step 1

### 3-[1-Dimethylamino-methylidene]-4-oxo-pyrrolidine-1-carboxylic acid tert-butyl ester

A mixture of 15.72 g (84.87 mmol) of N-(tert-butoxycarbonyl)-3-pyrrolidinone and 82 mL of N,N-dimethylformamide dimethylacetal is heated up at reflux for 1.5 hour. Excess of N,N-dimethylformamide dimethylacetal is removed in vacuo, the residue is triturated with n-hexane to provide a solid that is dried in vacuo; MS (method D): 241 [M+1]

### Step 2

### 2-(4-Methyl-piperazin-1-yl)-5,7-dihydro-pyrrolo[3,4-d]pyrimidine-6-carboxylic acid tert-butyl ester

A mixture of 0.39 g (1.62 mmol) of 3-[1-dimethylamino-methylidene]-4-oxo-pyrrolidine-1-carboxylic acid tert-butyl ester, 0.98 g (2.43 mmol) of 4-methylpiperazine-1-carboximidamide and 1.35 mL of sodium methoxide (5.4M in methanol) in 10 mL of ethanol is heated up at reflux overnight. The reaction mixture is poured into ice-water and extracted with EtOAc, the organics are washed with brine and dried over Na₂SO₄. Purification by RP-HPLC (method G) gives the title compound; MS (method D): 320 [M+1]

### Step 3

### 2-(4-Methyl-piperazin-1-yl)-6,7-dihydro-5H-pyrrolo[3,4-d]pyrimidine

A solution of 0.16 g (0.5 mmol) of 2-(4-methyl-piperazin-1-yl)-5,7-dihydro-pyrrolo[3,4-d]pyrimidine-6-carboxylic acid tert-butyl ester in 1 mL 1,4-dioxane is treated with 1.9 mL of 4N HCl in dioxane and stirred at RT under completion. The reaction mixture is concentrated in vacuo, taken up in 2N NaOH aqueous solution and extracted with EtOAc. The organics are dried over Na₂SO₄ and concentrated in vacuo to give the title compound; MS (method D): 220 [M+1]

The Following Compounds are Prepared in an Analogous Manner
(6,7-Dihydro-5H-pyrrolo[3,4-d]pyrimidin-2-yl)-dimethyl-amine: MS (method D): 165 [M+1]
2-Pyrrolidin-1-yl-6,7-dihydro-5H-pyrrolo[3,4-d]pyrimidine: MS (method D): 191 [M+1]
2-Morpholin-4-yl-6,7-dihydro-5H-pyrrolo[3,4-d]pyrimidine: MS (method D): 207 [M+1]

### Preparation of (6,7-dihydro-5H-pyrrolo[3,4-d]pyrimidin-4-yl)-dimethyl-amine

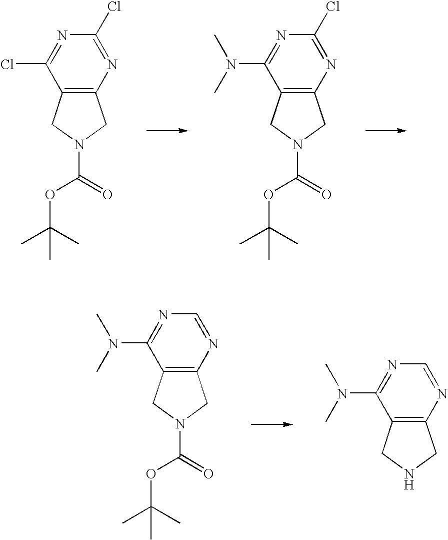

### Step 1

### 2-Chloro-4-dimethylamino-5,7-dihydro-pyrrolo[3,4-d]pyrimidine-6-carboxylic acid tert-butyl ester

A solution of 0.2 g (0.66 mmol) of 2,4-dichloro-5,7-dihydro-pyrrolo[3,4-d]pyrimidine-6-carboxylic acid tert-butyl ester in 8 mL of ethanol is treated with 0.103 mL (0.73 mmol) of triethylamine and 0.118 mL of a dimethylamine solution in ethanol (5.6 M). The vial is sealed and the reaction mixture is stirred at RT for 3 hours. The solvent is removed in vacuo and the residue is chromatographed on silica gel (eluent Hexane/EtOAc 4:1) to give the title compound; MS (method D): 299 [M+1], R_{f} 0.25 (eluent Hexane/EtOAc 3:1)

### Step 2

### 4-Dimethylamino-5,7-dihydro-pyrrolo[3,4-d]pyrimidine-6-carboxylic acid tert-butyl ester

A solution of 0.08 g (0.27 mmol) of 2-chloro-4-dimethylamino-5,7-dihydro-pyrrolo[3,4-d]pyrimidine-6-carboxylic acid tert-butyl ester in 10 mL of methanol is treated with 4 mL of triethylamine and degassed. Pd on Carbon (10%, 20 mg) is added and the reaction is allowed to stir overnight under an H₂ atmosphere. Under completion the catalyst is removed by filtration and the filtrate is chromatographed on silica gel (eluent Hexane/EtOAc 1:1) to afford the title compound; MS (method D): 265 [M+1]

### Step 3

### (6,7-Dihydro-5H-pyrrolo[3,4-d]pyrimidin-4-yl)-dimethyl-amine

A solution of 0.067 g (0.25 mmol) of 4-dimethylamino-5,7-dihydro-pyrrolo[3,4-d]pyrimidine-6-carboxylic acid tert-butyl ester in 1 mL of 1,4-dioxane is treated with 0.95 mL of 4N HCl in 1,4-dioxane. Under completion the reaction mixture is freeze-dried to give the title compound; MS (method D): 165 [M+1]

### Preparation of (S)-3-(3-chloro-phenyl)-1-oxa-2,7-diaza-spiro[4.4]non-2-ene-7,8-dicarboxylic acid 7-tert-butyl ester

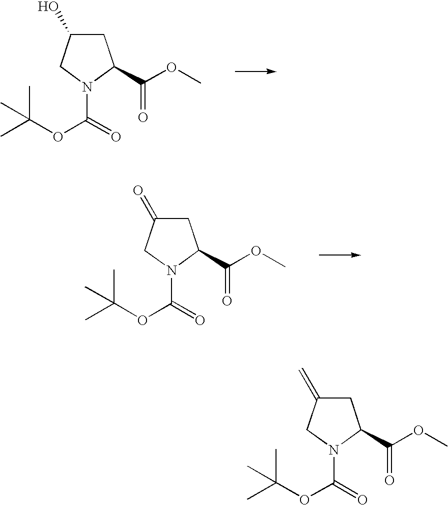

### Step 1

### (S)-4-Oxo-pyrrolidine-1,2-dicarboxylic acid 1-tert-butyl ester 2-methyl ester

A solution of 2.14 g (10.01 mmol) of sodium metaperiodate in 25 mL of water is added to a well stirred suspension of 0.168 g (1.26 mmol) of ruthenium(IV)oxide hydrate in 10 mL CCl₄ at 0° C. to give a yellow organic phase. A solution of 1.23 g (5.02 mmol) of Boc-Cis-HYP-OMe in chloroform is added in one portion. The ice bath is removed and the reaction mixture is allowed to stir at RT for 1.5 hour. The organic layer is separated, the water phase is extracted with ethylether. The organics are treated with 2-propanol, dried over Na₂SO₄, filtered over Celite and concentrated in vacuo to afford the title compound; MS (method D): 242 [M−1]

### Step 2

### (S)-4-Methylene-pyrrolidine-1,2-dicarboxylic acid 1-tert-butyl ester 2-methyl ester

A suspension of 0.3 g (2.59 mmol) of potassium tert-butoxide 20 mL of ethylether at 0° C. is treated with 0.944 g (2.59 mmol) of methyl-triphenylphosphoniumbromide, followed by 0.45 g (1.85 mmol) of (S)-4-oxo-pyrrolidine-1,2-dicarboxylic acid 1-tert-butyl ester 2-methyl ester after 15 minutes. The resulting brown mixture is heated up to reflux for 4 hours, poured into an ice-cold solution of ammonium chloride, and extracted with ethylether. The organic phase is dried over Na₂SO₄, concentrated and chromatographed on silica gel (eluent Hexane/EtOAc 6:1) to give the title compound; R_{f} 0.44 (eluent Hexane/EtOAc 3:1)

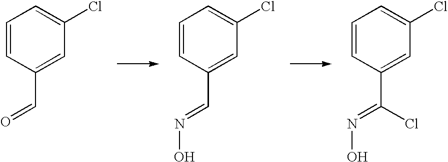

### Step 1

### 3-Chloro-benzaldehyde oxime

To a solution of 7.24 g (51.51 mmol) of 3-chlorobenzaldehyde and 3.941 g (56.14 mmol) of hydroxylamine hydrochloride in water (13 mL) and ethanol (13 mL) is added ice (25 g), followed by a 50% NaOH solution (5 mL). The resulting solution is stirred for 1 hour, acidified with concentrated HCl, and extracted with CH₂Cl₂. The organics are washed with water, dried over Na₂SO₄ and concentrated to give the title compound; MS (method D): 154 [M−1]

### Step 2

### 3-Chlorobenzohydroximinoyl chloride

A mixture of 0.5 g (3.21 mmol) of 3-chloro-benzaldehyde oxime and 0.447 g (3.21 mmol) of N-chlorosuccinimide in 5 mL DMF is stirred at 60° C. for 45 min. The reaction mixture is poured into ice-water, extracted with ethylether. The organics are washed with brine, dried over Na₂SO₄ and concentrated in vacuo to give the title compound; HPLC (method A) t_{R} (min) 4.17

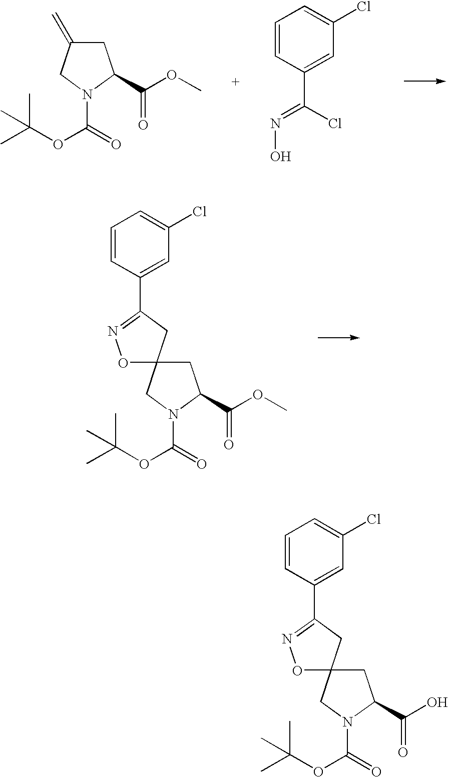

### Step 1

### (S)-3-(3-Chloro-phenyl)-1-oxa-2,7-diaza-spiro[4.4]non-2-ene-7,8-dicarboxylic acid 7-tert-butyl ester 8-methyl ester

A solution of 0.15 g (0.62 mmol) of (S)-4-methylene-pyrrolidine-1,2-dicarboxylic acid 1-tert-butyl ester 2-methyl ester in 10 mL of EtOAc is treated with 0.154 g (0.81 mmol) of 3-chlorobenzohydroximinoyl chloride below 7° C., followed by 0.114 mL (0.81 mmol) of triethylamine. The reaction mixture is stirred at RT overnight, poured into ice-water/EtOAc.

The organics are washed with brine, dried over Na₂SO₄, concentrated and chromatographed to give the title compound; HPLC (method A) t_{R} (min) 4.8 and 4.9 (4:1 ratio)

### Step 2

### (S)-3-(3-Chloro-phenyl)-1-oxa-2,7-diaza-spiro[4.4]non-2-ene-7,8-dicarboxylic acid 7-tert-butyl ester

A solution of 0.12 g (0.30 mmol) of (S)-3-(3-chloro-phenyl)-1-oxa-2,7-diaza-spiro[4.4]non-2-ene-7,8-dicarboxylic acid 7-tert-butyl ester 8-methyl ester in methanol (3 mL) and water 1.5 mL) is treated with 0.371 g (15.2 mmol) of LiOH and stirred at RT for 1 hour. The reaction mixture is poured into 6N HCl, extracted with CH₂Cl₂. The organics are combined, dried over Na₂SO₄ and concentrated to give the title compound; MS (method D): 379 [M−1]

The Following Compound is Prepared in an Analogous Manner:

### (S)-3-Pyridin-3-yl-1-oxa-2,7-diaza-spiro[4.4]non-2-ene-7,8-dicarboxylic acid 7-tert-butyl ester: MS (method D): 348 [M+1]

### Preparation of (2S,4R)-4-(6-chloro-benzo[d]isoxazol-3-yloxy)-pyrrolidine-1,2-dicarboxylic acid 1-tert-butyl ester

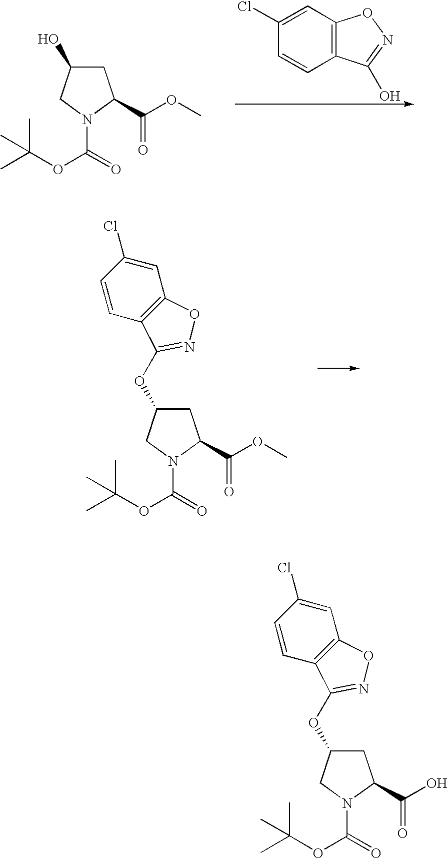

### Step 1

### (2S,4R)-4-(6-Chloro-benzo[d]isoxazol-3-yloxy)-pyrrolidine-1,2-dicarboxylic acid 1-tert-butyl ester 2-methyl ester

A solution of 1 g (4.08 mmol) of Boc-cis-HYP-OMe in 70 mL of THF is cooled to 0° C., treated with 0.784 g (4.48 mmol) of 6-chlorobenzo(d)isoxazol-3-ol, 1.62 g (6.12 mmol) of triphenylphosphine and after 5 minutes, 1.26 mL (6.12 mmol) of diisopropyl azodicarboxylate. The reaction mixture is stirred at RT overnight, concentrated and chromatographed by RP-HPLC (method G) to give the title compound; MS (method D): 297 [M-Boc+1]

### Step 2

### (2S,4R)-4-(6-Chloro-benzo[d]isoxazol-3-yloxy)-pyrrolidine-1,2-dicarboxylic acid 1-tert-butyl ester

A solution of 1.24 g (3.12 mmol) of (2S,4R)-4-(6-chloro-benzo[d]isoxazol-3-yloxy)-pyrrolidine-1,2-dicarboxylic acid 1-tert-butyl ester 2-methyl ester in methanol (3 mL) and water 1.5 mL) is treated with 0.382 g (15.6 mmol) of LiOH and stirred at RT for 1 hour. The reaction mixture is poured into 6N HCl, extracted with CH₂Cl₂. The organics are combined, dried over Na₂SO₄ and concentrated to give the title compound; MS (method D): 381 [M−1]

The Following Compounds are Prepared in an Analogous Manner:
(2S,4R)-4-(Isoxazolo[4,5-b]pyridin-3-yloxy)-pyrrolidine-1,2-dicarboxylic acid 1-tert-butyl ester: MS (method D): 348 [M−1]
(2S,4R)-4-(Isoxazolo[5,4-c]pyridin-3-yloxy)-pyrrolidine-1,2-dicarboxylic acid 1-tert-butyl ester: MS (method D): 348 [M−1]

### Preparation of (1R,2R)-1-tert-butoxycarbonylamino-2-ethyl-cyclopropanecarboxylic acid methyl ester

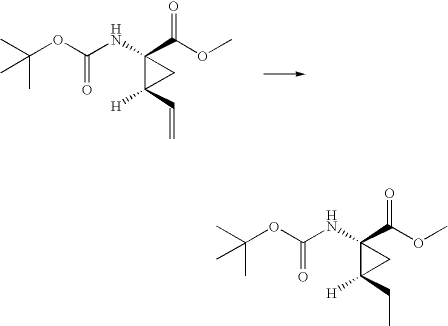

A solution of 15.94 g (66 mmol) of (1R,2S)-1-tert-butoxycarbonylamino-2-vinyl-cyclopropanecarboxylic acid methyl ester in 300 mL t-butyl-methyl ether is hydrogenated over 1.6 g of Pd(OH)₂ on Carbon (20%, wet) under H₂ atmosphere at RT, and under atmospheric pressure. The catalyst is filtered-off and the residue concentrated in vacuo to give the title compound; MS (method D): 242 [M−1]

### Biological Activity

### Example 227

### HCV NS3-4A Protease Assay

The inhibitory activity of certain compounds of Table A against HCV NS3-4A serine protease is determined in a homogenous assay using the full-length NS3-4A protein (genotype 1a, strain HCV-1) and a commercially available internally-quenched fluorogenic peptide substrate as described by Taliani, M., et al. 1996 Anal. Biochem. 240:60-67, which is incorporated by reference in its entirety.

### Example 228

### Luciferase-Based HCV Replicon Assay

The antiviral activity and cytotoxicity of certain compounds of Table A is determined using a subgenomic genotype 1b HCV replicon cell line (Huh-Luc/neo-ET) containing a luciferase reporter gene, the expression of which is under the control of HCV RNA replication and translation. Briefly, 5,000 replicon cells are seeded in each well of 96-well tissue culture plates and are allowed to attach in complete culture media without G418 overnight. On the next day, the culture media are replaced with media containing a serially diluted compound of Table A in the presence of 10% FBS and 0.5% DMSO. After a 48-h treatment with the compound of Table A, the remaining luciferase activities in the cells are determined using BriteLite reagent (Perkin Elmer, Wellesley, Mass.) with a LMaxII plate reader (Molecular Probe, Invitrogen). Each data point represents the average of four replicates in cell culture. IC₅₀ is the concentration of the at which the luciferase activity in the replicon cells is reduced by 50%. The cytotoxicity of the compound of Table A is evaluated using an MTS-based cell viability assay.

Compounds Table A supra have been tested in at least one of the protease assay of Example 227 or the replicon assay of Example 228 and exhibit an IC₅₀ of less than about 10 μM or less in at least one of the assays recited in Example 227 and 228.

### Equivalents

Those skilled in the art will recognize, or be able to ascertain using no more than routine experimentation, many equivalents to the specific embodiments and methods described herein. Such equivalents are intended to be encompassed by the scope of the following claims.

1. A compound of formula I: 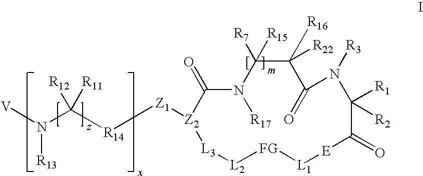
and pharmaceutically acceptable salts, enantiomers, stereoisomers, rotamers, tautomers, diastereomers, or racemates thereof;
wherein
the macrocycle: 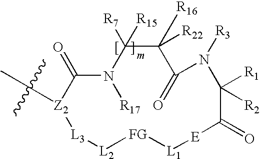
comprises between 15 to 40 ring atoms;
m, x and z are each independently selected from 0 or 1;
p is selected at each occurrence from the group consisting of 0, 1 and 2;
R₁ and R₂ are independently selected, at each occurrence, from hydrogen or cyano, or from the group consisting of alkyl, alkenyl, alkynyl, cycloalkyl, cycloalkylalkyl, alkoxy, and cycloalkyloxy, each of which is unsubstituted or substituted with 1-6 moieties which can be the same or different and are independently selected from the group consisting of hydroxy, oxo, alkyl, aryl, alkoxy, aryloxy, thio, alkylthio, arylthio, amino, alkylamino, arylamino, alkylsulfonyl, arylsulfonyl, alkylsulfonamido, arylsulfonamido, heteroarylsulfonamido, arylaminosulfonyl, heteroarylaminosulfonyl, mono and dialkylaminosulfonyl, carboxy, carbalkoxy, amido, carboxamido, alkoxycarbonylamino, aminocarbonyloxy, alkoxycarbonyloxy, alkylureido, arylureido, halogen, cyano, or nitro; wherein each of said alkyl, alkoxy, and aryl can be unsubstituted or optionally independently substituted with one or more moieties which can be the same or different and are independently selected from alkyl, alkenyl, alkynyl, cycloalkyl, cycloalkyl-alkyl, heterocyclyl, heterocyclylalkyl, aryl, alkylaryl, aralkyl, arylheteroaryl, heteroaryl, heterocyclylamino, alkylheteroaryl and heteroaralkyl;
R₃ is selected from the group consisting of H and C₁₋₄-alkyl;
E is a divalent residue selected from the group consisting of C(O)NR₂₃, NR₂₃S(O)ₚ, NR₂₃S(O)ₚNR₂₃;
L₁ and L₂ are divalent residues independently selected from the group consisting of C₀₋₄alkylene, (CH₂)ᵢ—FG—(CH₂)ₖ, (CH₂)ᵢ—C₃₋₇cycloalkylene-(CH₂)ₖ, (CH₂)ᵢ—C₃₋₇cycloheteroalkylene-(CH₂)ₖ, alkenylene, alkynylene, arylene, heteroarylene, cycloalkylene and heterocycloalkylene, each of which is substituted with 0 to 4 independently selected X₁ or X₂ groups;
i and k are independently selected integers of from 0 to 7;
L₃ is a C₀₋₄alkylene or a divalent ethylene or acetylene residue, wherein the C₀₋₄alkylene and divalent ethylene residues are substituted by 0-2 substituents selected from alkyl, aryl, heteroaryl, mono- or di-alkylamino-C₀-C₆alkyl, hydroxyl alkyl or alkoxyalkyl;
FG is absent or a divalent residue selected from the group consisting of O, S(O)ₚ, NR₂₃, C(O), C(O)NR₂₃, NR₂₃C(O), OC(O)NR₂₃, NR₂₃C(O)O, NR₂₃C(O)NR₂₃, S(O)ₚNR₂₃, NR₂₃S(O)ₚ, and NR₂₃S(O)ₚNR₂₃;
R₂₃ is independently selected at each occurrence from hydrogen or the group consisting of alkyl, alkenyl, alkynyl, cycloalkyl, cycloalkylalkyl, aryl, heteroaryl, heteroaralkyl, aralkyl and heteroaralkyl, each of which is substituted with 0-2 substituents independently selected from halogen, alkyl, alkoxy, and mono- and di-alkylamino; or
Two R₂₃ residues, taken in combination, form a monocyclic, bicyclic or tricyclic heterocyclic ring system which is saturated, partially unsaturated, or aromatic, and which is substituted with 0 to 3 substituents independently selected from C₁₋₆alkyl, C₁₋₆alkoxy, C₁₋₆alkoxyC₁₋₆alkoxy, mono- and di-C₁₋₆alkylaminoC₁₋₆alkoxy, C₁₋₆haloalkyl, C₁₋₆haloalkoxy, mono- and di-C₁₋₆alkylamino, halogen, 4 to 7 member heterocycloalkyl, aryl, heteroaryl, and 3 to 6 member spirocycloalkyl or spiroheterocycloalkyl, each of which is substituted with 0 to 3 substituents independently selected from the group consisting of C₁₋₄alkyl, C₁₋₄alkoxy, hydroxy, amino, and mono- and di-C₁₋₄alkylamino;
R₇, R₁₀, R₁₁, R₁₂, R₁₃, R₁₅, R₁₆, R₁₇, and R₂₂ are each, independently, selected from hydrogen or the group consisting of alkyl, alkenyl, alkynyl, aryl, alkyl-aryl, heteroalkyl, heterocyclyl, heteroaryl, aryl-heteroaryl, alkyl-heteroaryl, cycloalkyl, alkyloxy, alkyl-aryloxy, aryloxy, heteroaryloxy, heterocyclyloxy, cycloalkyloxy, amino, alkylamino, arylamino, alkyl-arylamino, arylamino, heteroarylamino, cycloalkylamino, carboxyalkylamino, aralkyloxy and heterocyclylamino; all of which may be further substituted 0 to 5 times with substituents independently selected from X₁ and X₂;
R₉ is absent or selected from hydrogen, C₁₋₄alkyl, C₃₋₇cycloalkyl-C₀₋₄alkyl, or hydroxy;
X₁ is alkyl, alkenyl, alkynyl, cycloalkyl, cycloalkyl-alkyl, heterocyclyl, heterocyclylalkyl, aryl, alkylaryl, aralkyl, arylheteroaryl, heteroaryl, heterocyclylamino, alkylheteroaryl, or heteroaralkyl; wherein X₁ can be independently substituted with one or more of X₂ moieties which can be the same or different and are independently selected;
X₂ is hydroxy, oxo, alkyl, aryl, heteroaryl, alkoxy, aryloxy, heteroaryloxy, thio, alkylthio, arylthio, heteroarylthio, amino, alkylamino, arylamino, heteroarylamino, alkylsulfonyl, arylsulfonyl, heteroarylsulfonyl, alkylsulfonamido, arylsulfonamido, heteroarylsulfonamido, arylaminosulfonyl, heteroarylaminosulfonyl, mono and dialkylaminosulfonyl, carboxy, carbalkoxy, amido, carboxamido, alkoxycarbonylamino, aminocarbonyloxy, alkoxycarbonyloxy, carbamoyl, ureido, alkylureido, arylureido, halogen, cyano, or nitro; wherein each of said alkyl, alkoxy, and aryl can be unsubstituted or optionally independently substituted with one or more moieties which can be the same or different and are independently selected from alkyl, alkenyl, alkynyl, cycloalkyl, cycloalkyl-alkyl, heterocyclyl, heterocyclylalkyl, aryl, alkylaryl, aralkyl, arylheteroaryl, heteroaryl, heterocyclylamino, alkylheteroaryl and heteroaralkyl;
Z₁ is C₀₋₄alkylene, oxygen or NR₁₀;
Z₂ is CR₉, O or N;
R₁₄ is C(O) or S(O)ₚ;
V is selected from hydrogen or from the group consisting of alkyl, alkyl-aryl, heteroalkyl, heterocyclyl, heteroaryl, aryl-heteroaryl, alkyl-heteroaryl, cycloalkyl, alkyloxy, alkyl-aryloxy, aryloxy, heteroaryloxy, heterocyclyloxy, cycloalkyloxy, amino, alkylamino, arylamino, alkyl-arylamino, arylamino, heteroarylamino, cycloalkylamino, carboxyalkylamino, mono- and di-alkylcarboxamide, aralkyloxy and heterocyclylamino; each of which may be further independently substituted one or more times with X¹ and X²; wherein X¹ is alkyl, alkenyl, alkynyl, cycloalkyl, cycloalkyl-alkyl, heterocyclyl, heterocyclylalkyl, aryl, alkylaryl, aralkyl, aryloxy, arylthio, arylheteroaryl, heteroaryl, heterocyclylamino, alkylheteroaryl, or heteroaralkyl; wherein X¹ can be independently substituted with one or more X² moieties which can be the same or different and are independently selected; wherein X² is hydroxy, oxo, alkyl, cycloalkyl, spirocycloalkyl, heterocycloalkyl, aryl, heteroaryl, alkoxy, aryloxy, thio, alkylthio, amino, mono- and di-alkylamino, arylamino, alkylsulfonyl, arylsulfonyl, alkylsulfonamido, arylsulfonamido, carboxy, carbalkoxy, carboxamido, alkoxycarbonylamino, alkoxycarbonyl, alkoxycarbonyloxy, alkylureido, arylureido, halogen, cyano, or nitro; wherein each X₂ residue selected to be alkyl, alkoxy, and aryl can be unsubstituted or optionally independently substituted with one or more moieties which can be the same or different and are independently selected from alkyl, alkenyl, alkynyl, cycloalkyl, cycloalkyl-alkyl, heterocyclyl, heterocyclylalkyl, aryl, alkylaryl, aralkyl, arylheteroaryl, heteroaryl, heterocyclylamino, alkylheteroaryl and heteroaralkyl;
or V is selected from the group consisting of -Q¹-Q², wherein Q¹ is absent, C(O), S(O)₂, N(H), N(C₁₋₄-alkyl), C═N(CN), C═N(SO₂CH₃), C═N—COH—C₁₋₄-alkyl, or C═N—COH, and Q² is hydrogen or is selected from the group consisting of C₁₋₄-alkyl, O—C₁₋₄-alkyl, NH₂, N(H)—C₁₋₄-alkyl, N(C₁₋₄-alkyl)₂, SO₂-aryl, SO₂-heteroaryl, SO₂—C₁₋₄-alkyl, C₃₋₆-cycloalkyl-C₀₋₄-alkyl, aryl, heteroaryl and heterocycle, each of which may be independently substituted one or more times with a halogen atom, C₁₋₄-alkyl, C₁₋₄-alkyl substituted by one or more halogen atoms, or C₃₋₆-cycloalkyl;
or R₂₂ and R₁₆ may together form a 3, 4, 5, 6 or 7-membered ring and may contain one or more heteroatoms, wherein the ring may be further substituted one or more times;
or R₇ and R₁₅ may together form a 3, 4, 5, 6 or 7-membered ring and may contain one or more heteroatoms, wherein the ring may be further substituted one or more times;
or R₁₅ and R₁₇ may together form a 3, 4, 5, 6 or 7-membered ring and may contain one or more heteroatoms, wherein the ring may be further substituted one or more times;
or R₁₅ and R₁₆ may together form a 4, 5, 6 or 7-membered ring and may contain one or more heteroatoms, wherein the ring may be further substituted one or more times;
or R₁₅ and R₁₆ may together form an arylene or heteroarylene ring and R₇ and R₂₂ are absent, wherein the ring may be further substituted one or more times;
or R₁ and R₂ may together form a 3, 4, 5, 6 or 7-membered ring that is saturated or partially unsaturated and may contain one or more heteroatoms, which ring is substituted with 0-3 residues independently selected from C₁₋₄alkyl, C₁₋₄alkoxy, C₂₋₄alkenyl, C₂₋₄alkynyl, halogen, hydroxy, C₃₋₆cylcoalkyl and C₃₋₆spirocycloalkyl;
or R₁₇ and R₁₆ may together form a 4, 5, 6, 7 or 8-membered ring of the formula: 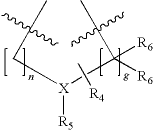
wherein
n and g are each, independently, 0, 1 or 2;
X is O, S, N, C or CR₅ₐ;
R₄ is hydrogen or is selected from the group consisting of C₁₋₆-alkyl, C₃₋₇-cycloalkyl, aryl, heterocycle and heteroaryl, all of which may be independently substituted one or more times with a halogen atom or C₁₋₄-alkyl;
R₅ is absent, hydrogen or oxo or is selected from the group consisting of hydroxyl, C₁₋₈-alkyl, C₂₋₈-alkenyl, C₂₋₈-alkynyl, C₃₋₈-cycloalkyl-C₀₋₄-alkyl, aryl-C₀₋₄-alkyl, heterocycle-C₀₋₄-alkyl, heteroaryl-C₀₋₄-alkyl, C₃₋₈-cycloalkyloxy, aryloxy, NR₂₃COR₂₃, CONR₂₃R₂₃, NR₂₃CONHR₂₃, OCONR₂₃R₂₃, NR₂₃COOR₂₃, OCOR₂₃, COOR₂₃, aryl-C(O)O, aryl-C(O)NR₂₃, heteroaryloxy, heteroaryl-C(O)O, heterocycle-C(O)O, heteroaryl-C(O)NR₂₃, heterocycle-C(O)NR₂₃, each of which may be independently substituted one or more times (or more preferably 0, 1, 2, 3, 4, or 5 times) with halogen, C₁₋₄-alkyl, C₁₋₄-alkoxy, haloC₁₋₄-alkoxy, amino, mono- and di-C₁₋₄alkylaminoC₀₋₄alkyl, mono- and di-C₁₋₄alkylaminoC₀₋₄alkoxy, C₃₋₇cycloalkyl, fused- or spiro-cyclic 3-7 membered ring, heterocycleC₀₋₄alkoxy, heterocycleC₀₋₄alkyl, aryl, or heteroaryl;
R₅ₐ is selected from the group consisting of H, hydroxyl, C₁₋₈-alkyl, C₂₋₈-alkenyl, C₂₋₈-alkynyl, C₃₋₈-cycloalkyl-C₀₋₄alkyl, aryl-C₀₋₄-alkyl and heteroaryl-C₀₋₄-alkyl,
or R₄ and R₅ may together form a fused dimethyl cyclopropyl ring, a fused cyclopentane ring, a fused phenyl ring or a fused pyridyl ring, each of which may be substituted with a halogen atom, aryl, heteroaryl, trihalomethyl, C₁₋₄-alkoxy or C₁₋₄-alkyl;
or R₅ and R₅ₐ may together form a spirocyclic ring having between 3 and 7 ring atoms and having 0, 1, or 2 ring heteroatoms, which is optionally substituted by 0-4 substitutents selected from cyano, halogen, hydroxyl, amino, thiol, C₁₋₈-alkyl, C₂₋₈-alkenyl, C₂₋₈-alkynyl, C₁₋₈-alkoxy-C₀₋₄alkyl, C₁₋₈-haloalkyl, C₂₋₈-haloalkenyl, C₂₋₈-haloalkynyl, C₁₋₈-haloalkoxy, C₁₋₈-alkylthio, C₁₋₈-alkylsulfonyl, C₁₋₈-alkylsulfoxy, C₁₋₈-alkanoyl, C₁₋₈-alkoxycarbonyl, C₃₋₇-cycloalkyl-C₀₋₄-alkyl, aryl-C₀₋₄-alkyl, heteroaryl-C₀₋₄-alkyl, COON, C(O)NH₂, mono- and di-C₁₋₄-alkyl-carboxamide, mono- and di-C₁₋₄-alkyl-amino-C₀₋₄alkyl, SO₃H, SO₂NH₂, and mono-and di-C₁₋₄-alkylsulfonamide, or two substitutents taken together form a fused or spirocyclic 3 to 7 membered ring having 0, 1 or 2 ring heteroatoms selected from N, O and S, which fused or spirocyclic ring has 0 to 2 independently selected substitutents selected from cyano, halogen, hydroxyl, amino, thiol, C₁₋₈-alkenyl, C₂₋₈-alkynyl, C₂₋₈-alkynyl, C₁₋₈-alkoxy-C₀₋₄alkyl, C₁₋₈-haloalkyl, C₂₋₈-haloalkenyl, C₂₋₈-haloalkynyl, C₁₋₈-haloalkoxy, C₁₋₈-alkylthio, C₁₋₈-alkylsutfonyl, C₁₋₈-alkylsulfoxy, C₁₋₈-alkanoyl, C₁₋₈-alkoxycarbonyl, C₃₋₇-cycloalkyl-C₀₋₄-alkyl, aryl-C₀₋₄-alkyl, heteroaryl-C₀₋₄-alkyl, COON, C(O)NH₂, mono- and di-C₁₋₄-alkyl-carboxamide, mono- and di-C₁₋₄-alkyl-amino-C₀₋₄alkyl, SO₃H, SO₂NH₂, and mono-and di-C₁₋₄-alkylsulfonamide; and
R₆ is independently selected at each occurrence from the group consisting of hydrogen, hydroxy, amino, C₁₋₄alkyl, C₁₋₄alkoxy, and mono- and di-C₁₋₄alkylamino, and C₃₋₆cycloalkylC₀₋₄alkyl;
or two R₆ residues may together form a spirocyclic ring having between 3 and 7 ring atoms and having 0, 1, or 2 ring heteroatoms, which is optionally substituted by 0-4 substitutents selected from cyano, halogen, hydroxyl, amino, thiol, C₁₋₈-alkyl, C₂₋₈-alkenyl, C₂₋₈-alkynyl, C₁₋₈-alkoxy-C₀₋₄alkyl, C₁₋₈-haloalkyl, C₂₋₈-haloalkenyl, C₂₋₈-haloalkynyl, C₁₋₈-haloalkoxy, C₁₋₈-alkylthio, C₁₋₈-alkylsulfonyl, C₁₋₈-alkylsulfoxy, C₁₋₈-alkanoyl, C₁₋₈-alkoxycarbonyl, C₃₋₇-cycloalkyl-C₀₋₄alkyl, heteroaryl-C₀₋₄-alkyl, COON, C(O)NH₂, mono- and di-C₁₋₄-alkyl-carboxamide, mono- and di-C₁₋₄-alkyl-amino-C₀₋₄alkyl, SO₃H, SO₂NH₂, and mono-and di-C₁₋₄-alkylsulfonamide, or two substitutents taken together form a fused or spirocyclic 3 to 7 membered ring having 0, 1 or 2 ring heteroatoms selected from N, O and S, which fused or spirocyclic ring has 0 to 2 independently selected substitutents selected from halogen, C₁₋₄alkyl, C₁₋₄alkoxy, C₁₋₄alkanoyl, mono- and di-C₁₋₄-alkylamino, mono- and di-C₁₋₄-alkyl-carboxamide, C₁₋₄-alkoxycarbonyl, and phenyl.

2. (canceled)

3. A compound of claim 1 wherein R₂ and one occurrence of R₁ taken in combination form a cyclopropyl ring which is substituted with 0 or 1 substituents selected C₁₋₄alkyl, vinyl or cyclopropyl; E is C(O)NH, NHS(O)₂, NHSO₂N(Me), NHSO₂N(Et) or NHSO₂N(cyclopropyl).

4-9. (canceled)

10. A compound of claim 1 wherein the compound is a compound of formula II: 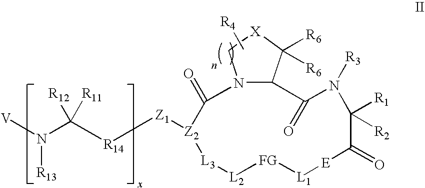
and pharmaceutically acceptable salts, enantiomers, stereoisomers, rotamers, tautomers, diastereomers, or racemates thereof.

11. The compound of claim 10, wherein
x is 0 or 1;
n is 0 or 1;
R₁₄ is C(O) or S(O)ₚ;
Z₁ is absent or NH;
Z₂ is nitrogen or CH;
R₁ is selected from the group consisting of H and C₁₋₄-alkyl;
R₂ is selected from the group consisting of C₁₋₄-alkyl, C(O)C₁₋₄-alkyl, C(O)OC₁₋₄-alkyl, and (CH₂)₀₋₄—C₃₋₆-cycloalkyl;
or R₁ and R₂ together form a cyclopropyl ring which is substituted with 0 or 1 substituents selected C₁₋₄alkyl, vinyl or cyclopropyl;
R₃ is selected from the group consisting of H and C₁₋₄-alkyl;
X is O, NR₅ or CR₅R₅ₐ;
R₄ is hydrogen or is selected from the group consisting of C₁₋₄-alkyl, C₃₋₆-cycloalkyl, aryl, heterocycle and heteroaryl, each of which may be independently substituted one or more times with a halogen atom or C₁₋₄-alkyl;
R₅ is hydrogen or oxo or is selected from the group consisting of hydroxyl, C₁₋₈-alkyl, C₂₋₈-alkenyl, C₂₋₈-alkynyl, C₃₋₈-cycloalkyl-C₀₋₄-alkyl, aryl-C₀₋₄-alkyl, aryloxy, heteroaryloxy, heterocycle-C₀₋₄-alkyl and heteroaryl-C₀₋₄-alkyl, each of which may be independently substituted one or more times with a halogen atom, aryl, heteroaryl, trihalomethyl, C₁₋₄-alkoxy or C₁₋₄-alkyl; or
R₅ is a residue of the formula: 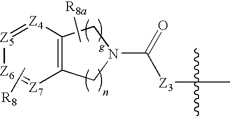
wherein
n and g are integers independently selected from 0, 1, or 2;
Z₃ is NR₂₃ or O;
Z₄, Z₅, Z₆, and Z₇ are each independently selected from the group consisting of N, CH, and CR₈;
R₈ and R₈ₐ each indepently represent 0 to 2 groups, each of which is independently selected at each occurrence of R₈ and R₈ₐ from the group consisting of hydrogen, halogen, C₁₋₄-alkyl, C₁₋₄-alkoxy, haloC₁₋₄-alkyl, haloC₁₋₄-alkoxy, amino, mono- and di-C₁₋₄alkylaminoC₀₋₄alkyl, mono- and di-C₁₋₄alkylaminoC₀₋₄alkoxy, heterocycleC₀₋₄alkoxy, heterocycleC₀₋₄alkylamino, and heterocycleC₀₋₄alkyl;
R₅ₐ is selected from the group consisting of H, hydroxyl, C₁₋₈-alkyl, C₂₋₈-alkenyl, C₂₋₈-alkynyl, C₃₋₈-cycloalkyl-C₀₋₄-alkyl, aryl-C₀₋₄-alkyl and heteroaryl-C₀₋₄-alkyl,
or R₄ and R₅ may together form a fused dimethyl cyclopropyl ring, a fused cyclopentane ring, a fused phenyl ring or a fused pyridyl ring, each of which may be substituted with a halogen atom, aryl, heteroaryl, trihalomethyl, C₁₋₄-alkoxy or C₁₋₄-alkyl;
or R₅ and R₅ₐ may together form a spirocarbocyclic saturated ring having between 3 and 6 carbon ring atoms which is optionally substituted by 0-2 substitutents selected from halogen, C₁₋₆-alkyl, C₂₋₆-alkenyl, C₂₋₆-alkynyl, C₁₋₆-alkoxide, C₃₋₇-cycloalkyl-C₀₋₄-alkyl, phenyl-C₀₋₄-alkyl, naphthyl-C₀₋₄alkyl, heteroaryl-C₀₋₄-alkyl, or two substitutents taken together form a fused or spirocyclic 3 to 7 membered carbocyclic ring, each of which is substituted with 0-3 independently selected halogen atoms or C₁₋₄-alkyl groups;
R₁₀ and R₁₁ are each, independently, selected from the group consisting of H and C₁₋₄-alkyl;
R₆ and R₁₃ is H;
R₁₂ is selected from the group consisting of H, C₁₋₄-alkyl and C₃₋₈-cycloalkyl; and
V is selected from the group consisting of -Q¹-Q², wherein Q¹ is absent, C(O), N(H), N(C₁₋₄-alkyl), C═N(CN), C═N(SO₂CH₃), or C═N—COH, and Q² is H, C₁₋₄-alkyl, C═N—COH—C₁₋₄-alkyl, C₁₋₄-alkoxy, C₃₋₇cycloalkyloxy, heterocycloalkyloxy, NH₂, N(H)—C₁₋₄-alkyl, N(C₁₋₄-alkyl)₂, SO₂-aryl, SO₂—C₁₋₄-alkyl, C₃₋₆cycloalkyl-C₀₋₄-alkyl, aryl, heteroaryl and heterocycle, each of which may be independently substituted one or more times with a halogen atom, C₁₋₄-alkyl, C₁₋₄alkoxy, C₂-C₄alkenyloxy, C₂-C₄alkynyloxy, C₁₋₄-alkyl substituted by one or more halogen atoms, or C₃₋₆-cycloalkyl;
or when x is 0, R₁₀ and V can form a cyclopropyl ring that may be further substituted by an amide group.

12-13. (canceled)

14. The compound of claim 10, wherein V is hydrogen or selected from R²⁰ or C(O)R²⁰, wherein R²⁰ is selected from the group consisting of 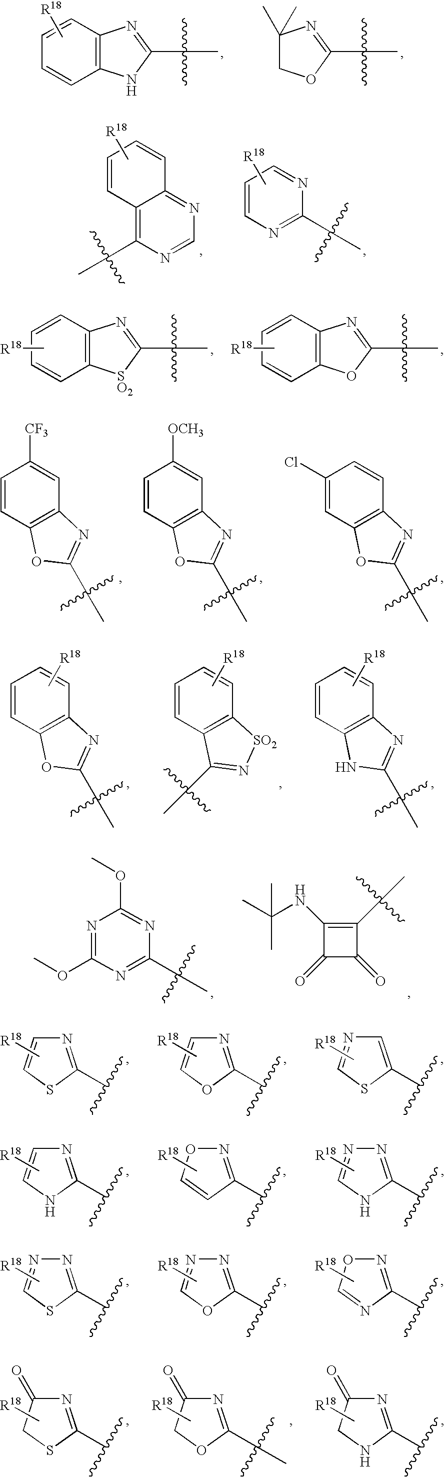 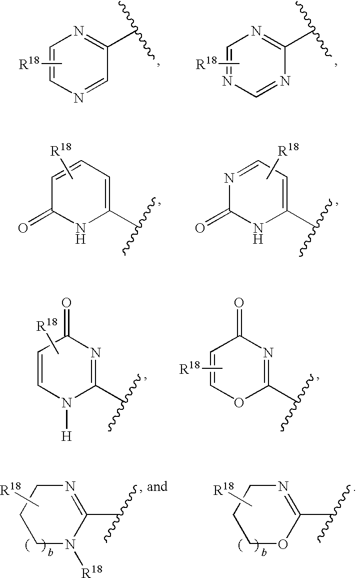
wherein b is 0, 1, or 2; and R₁₈ is selected from the group consisting of hydrogen, a halogen atom, aryl, trihalomethyl, and C₁₋₄-alkyl.

15-17. (canceled)

18. The compound of claim 10 according to Formula IIb: 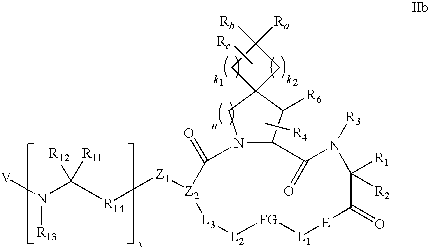
Z₂ is nitrogen or CH;
k₁ and k₂ are 0 or 1 such that a sum of k₁ and k₂ equals 1 or 2;
Rₐ and R_{b} taken together form a spirocyclic 3 to 6 membered ring having 0, 1 or 2 ring heteroatoms selected from N, O and S, which fused or spirocyclic ring has 0 to 2 independently selected substituents selected from halogen, C₁₋₄alkyl, C₁₋₄alkoxy, C₁₋₄alkanoyl, and phenyl;
R_{c} represents 0 to 2 substituents which are independently selected at each occurrence of R_{c} from the group consisting of halogen, C₁₋₄alkyl, and phenyl, or two geminal R_{c} substitents, taken in combination form a 3 to 6 member spirocyclic ring;
R₄ represents 0, 1, or 2 substituents each of which is independently selected from H and C₁₋₄-alkyl; and
R₆ is hydrogen or C₁₋₄alkyl.

19. The compound of claim 18, wherein the divalent residue: 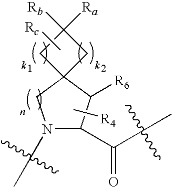
is selected from the group consisting of:

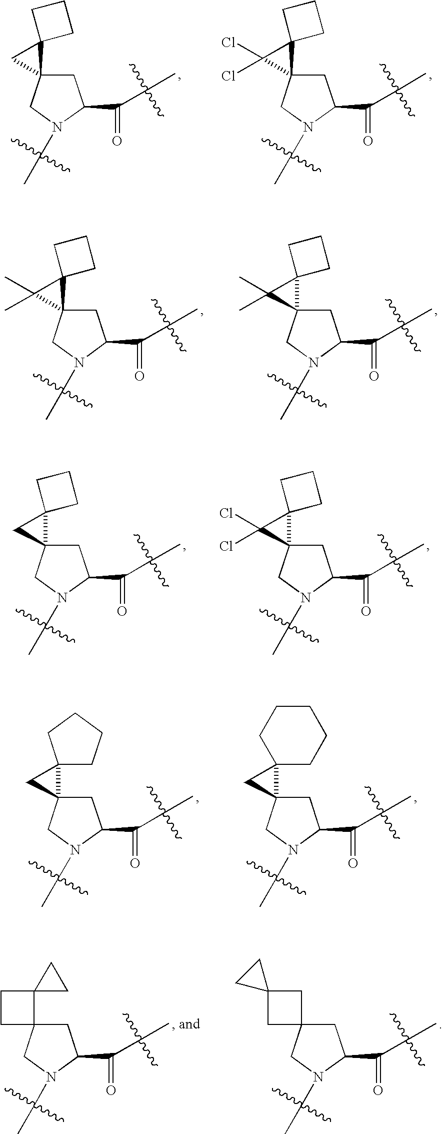

20. A compound of claim 1, wherein the compound is a compound of formula III: 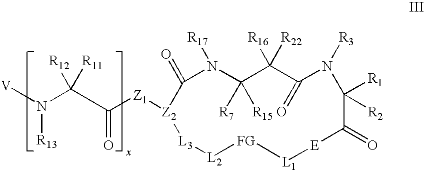
and pharmaceutically acceptable salts, enantiomers, stereoisomers, rotamers, tautomers, diastereomers, or racemates thereofs.

21. The compound of claim 20, wherein
Z₂ is nitrogen or CH;
Z₁ is absent or NR₁₀;
R₃ is selected from the group consisting of H, C₁₋₄-alkyl, and C₃₋₆-cycloalkylC₀-C₄alkyl;
R₁₁, R₁₅ and R₂₂ are selected from the group consisting of H, alkyl-aryl, C₁₋₄-alkyl, O—C₁₋₄-alkyl, N(H)—C₁₋₄-alkyl, and C₃₋₆-cycloalkylC₀-C₄alkyl;
R₁₀ and R₁₇ are each, independently, selected from the group consisting of H, C₁₋₄-alkyl and (CH₂)₀₋₄—C₃₋₆-cycloalkyl; or
R₁₅ and R₁₆ may together form a 3, 4, 5, 6 or 7-membered ring that may comprise between 0 to 3 additional heteroatoms, wherein the ring may be further substituted with 0-5 substitutents; or
R₁₆ and R₁₇ may together form a 3, 4, 5, 6 or 7-membered ring that may comprise between 0 to 3 additional heteroatoms, wherein the ring may be further substituted with 0-5 substitutents; and
V is selected from the group consisting of -Q¹-Q², wherein Q¹ is absent, C(O), N(H), N(C₁₋₄-alkyl), C═N(CN), C═N(SO₂CH₃), or C═N—COH, and Q² is H, C₁₋₄-alkyl, C═N—COH—C₁₋₄-alkyl, C₁₋₄-alkoxy, C₃₋₇cycloalkyloxy, heterocycloalkyloxy, NH₂, N(H)—C₁₋₄-alkyl, N(C₁₋₄-alkyl)₂, SO₂-aryl, SO₂—C₁₋₄-alkyl, C₃₋₆cycloalkyl-C₀₋₄-alkyl, aryl, heteroaryl and heterocycle, each of which may be independently substituted one or more times with a halogen atom, C₁₋₄-alkyl, C₁₋₄alkoxy, C₂-C₄alkenyloxy, C₂-C₄alkynyloxy, C₁₋₄-alkyl substituted by one or more halogen atoms, or C₃₋₆-cycloalkyl.

22-24. (canceled)

25. A compound of claim 1, wherein the compound is a compound of formula IV: 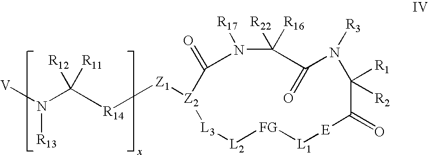
and pharmaceutically acceptable salts, enantiomers, stereoisomers, rotamers, tautomers, diastereomers, or racemates thereof.

26. The compound of claim 25, wherein
Z₂ is nitrogen or CH;
R₃ is selected from the group consisting of H and C₁₋₄-alkyl;
R₁₇ is selected from hydrogen or the group consisting of C₁₋₄-alkyl, C₁₋₆-cycloalkyl, (CH₂)₀₋₄—C₃₋₆-cycloalkyl, aryl, alkyl-aryl and heterocycle, each of which may be independently substituted one or more times;
R₁₀ and R₁₁ are each, independently, selected from the group consisting of H and C₁₋₄-alkyl;
R₁₂ is selected from the group consisting of H, C₁₋₄-alkyl, C₁₋₆-cycloalkyl and aryl; and
V is selected from the group consisting of -Q¹-Q², wherein Q¹ is absent, C(O), N(H), N(C₁₋₄alkyl), C═N(CN), C═N(SO₂CH₃), or C═N—COH, and Q² is H, C₁₋₄-alkyl, C═N—COH—C₁₋₄-alkyl, O—C₁₋₄-alkyl, NH₂, N(H)—C₁₋₄alkyl, N(C₁₋₄-alkyl)₂, SO₂-aryl, SO₂—C₁₋₄-alkyl, C₃₋₆-cycloalkyl-C₀₋₄-alkyl, aryl, heteroaryl and heterocycle, each of which may be independently substituted one or more times with a halogen atom, C₁₋₄-alkyl, C₁₋₄-alkyl substituted by one or more halogen atoms, or C₃₋₆-cycloalkyl;
or R₁₁ and V form the following 5-membered ring which may be further substituted:

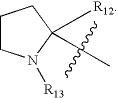

27-29. (canceled)

30. The compound of claim 1, wherein V is R₂₀ or C(O)—R₂₀, wherein R₂₀ is a residue of the formula: 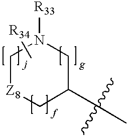
wherein
Z₈ is absent or selected from NR₃₃ or oxygen;
g and f are independently selected integers selected from the group consisting of 0, 1, 2, 3 and 4;
j is an integer selected from the group consisting of 1, 2, 3 and 4, wherein the sum of f+g+j is less than or equal to 5 and greater than or equal to 2 when Z₈ is absent and the sum of f+g+jk is less than or equal to 4 and greater than or equal to 1 when 4 is oxygen;
R³³ is independently selected at each occurrence from the group consisting of hydrogen, C₁₋₄alkyl, haloC₁₋₄alkyl, C₃₋₆cycloalkyl, hydroxyC₁₋₄alkyl, and C₁₋₄alkoxyC₁₋₄alkyl; and
R³⁴ represents zero to three residues each independently selected at each occurrence from the group consisting of halogen, hydroxy, amino, C₁₋₄alkyl, C₃₋₆cycloalkyl, C₁₋₄alkoxy, mono-and di-C₁₋₄alkylamino, hydroxyC₁₋₄alkyl, and C₁₋₄alkoxyC₁₋₄alkyl.

31. (canceled)

32. A pharmaceutical composition comprising at least one compound according to claim 1 and a pharmaceutically acceptable carrier.

33. The pharmaceutical composition of claim 32, wherein the composition further comprises at least one additional HCV-modulating compound.

34-38. (canceled)

39. A method of treating an HCV-associated disorder comprising administering to a subject in need thereof a pharmaceutically acceptable amount of a compound according to claim 1.

40. The method of claim 39, wherein the HCV-associated disorder is selected from the group consisting of HCV infection, liver cirrhosis, chronic liver disease, hepatocellular carcinoma, cryoglobulinaemia, non-Hodgkin's lymphoma, and a suppressed innate intracellular immune response.

41. A method of treating an HIV infection comprising administering to a subject in need thereof a pharmaceutically acceptable amount of a compound according to claim 1.

42. A method of treating, inhibiting or preventing the activity of HCV in a subject in need thereof, comprising administering to the subject a pharmaceutically acceptable amount of a compound according to claim 1.

43-54. (canceled)

55. A method of decreasing the HCV RNA load in a subject in need thereof comprising administering to the subject a pharmaceutically acceptable amount of a compound according to claim 1, such that the HCV RNA load in the subject is decreased.

56. (canceled)

57. A method of treating an HCV-associated disorder comprising administering to a subject in need thereof a pharmaceutically effective amount of a compound according to claim 1, in combination with a pharmaceutically effective amount of an additional HCV-modulating compound, such that the HCV-associated disorder is treated.

58-63. (canceled)

64. A method of inhibiting hepatitis C virus replication in a cell, comprising contacting said cell with a compound according to claim 1.

65-66. (canceled)

67. A method of treating HCV infection, liver cirrhosis, chronic liver disease, hepatocellular carcinoma, cryoglobulinaemia, non-Hodgkin's lymphoma, and/or a suppressed innate intracellular immune response in subject in need thereof comprising administering to the subject a pharmaceutically acceptable amount of a compound according to claim 1.

68-69. (canceled)

70. A method of preventing liver damage in a liver transplant patient, the method comprising administration of a compound of claim 1 to a patient who has received a liver transplant or is scheduled for a liver transplant operation.
